(19) United States
(12) Patent Application Publication
(10) Pub. No.: US 2024/0059797 A1
(43) Pub. Date: Feb. 22, 2024

(54) ENGINEERED MULTI-SPECIFIC ANTIBODIES AND RELATED METHODS OF USE AND MANUFACTURE

(71) Applicant: Gritstone bio, Inc., Emeryville, CA (US)

(72) Inventors: Godfrey Jonah Anderson Rainey, San Diego, CA (US); Karin Jooss, San Diego, CA (US); Shan Liu Hwang, Fremont, CA (US); Gayatri Prakash, El Cerrito, CA (US); Isaac J. Rondon, Emeryville, CA (US)

(21) Appl. No.: 18/018,400

(22) PCT Filed: Jul. 29, 2021
(86) PCT No.: PCT/US21/43796

§ 371 Date: Jan. 27, 2023

Related U.S. Application Data
(60) Provisional application No. 63/058,461, filed on Jul. 29, 2020.

(51) Int. Cl. C07K 16/46; A61P 35/00

(52) U.S. Cl. CPC C07K 16/468; A61P 35/00; C07K 2317/55; C07K 2317/524; C07K 2317/526; C07K 2317/31; C07K 2317/92

(57) ABSTRACT

Provided herein are multispecific antigen binding proteins (ABPs). Also provided herein are multispecific ABPs that are engineered to form diabodies or stay in extended (dual scFv) conformation and methods for purifying ABPs based on avidity.

### CROSS-REFERENCE TO RELATED APPLICATIONS

This application claims priority to and the benefit of U.S. Provisional Patent Application No. 63/058,461, filed on Jul. 29, 2020, the entire contents of which are herein incorporated by reference for all purposes.

### SEQUENCE LISTING

The instant application contains a Sequence Listing which will be submitted via EFS-Web and is hereby incorporated by reference in its entirety. Said ASCII copy, created on Jul. 29, 2021, is named GSO-093WO_SequenceListing.txt and is 512,684 bytes in size.

### BACKGROUND

Specific antigen recognition is essential for antibodies to function in the adaptive immune system. The specificity of antibodies and antibody fragments for a particular antigen or antigens makes antibodies desirable therapeutic agents. Antibodies and antibody fragments can be used to target specific tissues, for example, tumor tissue or infected tissue, thereby minimizing potential side effects of non-specific targeting. Thousands of antigens are capable of eliciting responses, each almost exclusively directed to the particular antigen which elicited it.

Tumor cells can express antigens and may display such antigens on the surface of the tumor cell. Such tumor antigens can be used for development of novel immunotherapeutic reagents for the specific targeting of tumor cells. For example, tumor antigens can be used to identify therapeutic antigen binding proteins, e.g., TCRs, antibodies, or antigen-binding fragments. Such tumor antigens may also be utilized in pharmaceutical compositions, e.g., vaccines.

Included in potential antigens for immunotherapeutic applications are major histocompatibility complex class I molecules. Major histocompatibility complex class I molecules are expressed on the surface of virtually all nucleated cells in the body and are dimeric molecules comprising a transmembrane heavy chain, comprising the peptide antigen binding cleft, and a smaller extracellular chain termed beta2-microglobulin. MHC class I molecules present peptides derived from the degradation of cytosolic proteins by the proteasome, a multi-unit structure in the cytoplasm, (Niedermann G., 2002. Curr Top Microbiol Immunol. 268:91-136; for processing of bacterial antigens, refer to Wick M J, and Ljunggren H G., 1999. Immunol Rev. 172:153-62, each of which is incorporated by reference in its entirety). Cleaved peptides are transported into the lumen of the endoplasmic reticulum (ER) by the transporter associated with antigen processing (TAP) where they are bound to the groove of the assembled class I molecule, and the resultant MHC/peptide complex is transported to the cell membrane to enable antigen presentation to T lymphocytes (Yewdell J W., 2001. Trends Cell Biol. 11:294-7; Yewdell J W. and Bennink J R., 2001. Curr Opin Immunol. 13:13-8, each of which is incorporated by reference in its entirety). Alternatively, cleaved peptides can be loaded onto MHC class I molecules in a TAP-independent manner and can also present extracellularly-derived proteins through a process of cross-presentation. As such, a given MHC/peptide complex presents a novel protein structure on the cell surface that can be targeted by a novel antigen-binding protein (e.g., antibodies or TCRs) once the identity of the complex's structure (peptide sequence and MHC subtype) is determined.

Conventional approaches to cancer treatment include chemotherapy, radiation therapy, and surgical removal of solid tumors or tumor-tissue. Recently, T cell-targeting therapeutic antibodies have been developed. These include bispecific antibodies, capable of simultaneously binding cell surface antigens on T cells and cell surface antigens on tumor cells, thereby enabling the bound T cells to contribute to the destruction of the tumor cells.

Isolated antibodies at high purity are in demand for therapeutic applications. As a result, various methods for purifying antibody and antibody fragments for therapeutic applications have been developed. In general, antibody purification involves the use of conventional chromatography, such as gel filtration, ion exchange, mixed-mode, or hydrophobic chromatography, and affinity chromatography. Efforts are underway to advance methods for purifying bispecific (and more broadly, multispecific) antibodies.

This application is related to PCT/US2020/15736, filed on Jan. 29, 2020, or U.S. application Ser. No. 17/426,627, filed Jul. 28, 2021, each of which is hereby incorporated by reference in its entirety for all purposes.

### SUMMARY

Provided herein are multispecific antigen binding proteins (ABPs) targeting a tumor antigen (e.g., pHLA). These ABPs can be engineered to form diabody structures through modulation of, e.g., linkers and disulfide bridges. The ABP are useful for treating disorders such as cancer and chronic viral disease.

In one aspect, provided herein is an isolated multispecific antigen binding protein (ABP) comprising a first antigen binding region (ABR) and a second ABR that each specifically bind a first target antigen, a Fab that specifically binds an additional target antigen that is distinct from the first target antigen, and an Fc domain, wherein the ABP comprises a first polypeptide, a second polypeptide, and a third polypeptide, wherein the first polypeptide comprises, in an N→C direction, the first ABR-a first hinge-CH2-CH3, wherein the second polypeptide comprises, in an N→C direction, a VH domain of the Fab-a CH1 domain of the Fab-a second hinge-CH2-CH3, wherein the third polypeptide comprises, in an N→C direction, a VL domain of the Fab-a CL domain of the Fab; wherein the second ABR is attached, directly or indirectly, to the N-terminus of the second polypeptide or the third polypeptide; wherein the first ABR and second ABR each comprise, in an N→C direction: (i) a VH domain-a VL domain or (ii) a VL domain-VH domain; wherein the VH domain of the first ABR is attached to the VL domain of the first ABR via a first linker; wherein the VH domain of the second ABR is attached to the VL domain of the second ABR via a second linker; wherein the first linker and second linker are each less than 20 amino acids in length. In some embodiments, the first linker and second linker are each 20 amino acids in length.

In some embodiments, the second ABR is attached, directly or indirectly, to the N-terminus of the second polypeptide, wherein the first target antigen is an HLA-PEPTIDE target, wherein the HLA-PEPTIDE target comprises an HLA-restricted peptide complexed with an HLA Class I molecule, wherein the additional target antigen is CD3, wherein the first and second linker are each 5-10 amino acids in length, wherein the CH2-CH3 of the first polypeptide and the CH2-CH3 of the second polypeptide comprise a variant CH2-CH3 domain, wherein the variant CH2-CH3 domains comprises the amino acid substitutions of L234F, L235E, and P331S, according to the EU numbering system, further comprising an S354C and T366W mutation in one variant CH2-CH3 domain and a Y349C, T366S, L368A and Y407V mutation in the other variant CH2-CH3 domain, according to EU numbering, wherein the first hinge comprises a C220S mutation, according to EU numbering, and wherein the CH2-CH3 of either the first polypeptide or the second polypeptide comprises a H435R_Y436F mutation, according to EU numbering.

In some embodiments, the CH2-CH3 of either the first polypeptide or the second polypeptide, but not both, comprises a H435R_Y436F mutation, according to EU numbering. In some embodiments, the CH2-CH3 of either the hole side of the antibody comprises a H435R_Y436F mutation, according to EU numbering. In alternate embodiments, the CH2-CH3 of either the knob side of the antibody comprises a H435R_Y436F mutation, according to EU numbering.

In some embodiments, the first linker and second linker are each less than 20 amino acids in length. In some embodiments, the first linker and the second linker each have a length of 10 amino acids and wherein the first linker and second linker each consists of (GGGGS)₂ (SEQ ID NO: 4). In some embodiments, the HLA Class I molecule is HLA subtype A*01:01 and the HLA-restricted peptide comprises the sequence NTDNNLAVY (SEQ ID NO: 5). In some embodiments, the HLA Class I molecule is HLA subtype A*02:01 and the HLA-restricted peptide comprises the sequence AIFPGAVPAA (SEQ ID NO: 6).

In some embodiments, one or both of the first linker and the second linker have a length of 10 amino acids or less. In some embodiments, one or both of the first linker and the second linker have a length of 8 amino acids or less. In some embodiments, one or both of the first linker and the second linker have a length of 5 amino acids or less.

In some embodiments, the first linker and second linker are each less than 14 amino acids in length. In some embodiments, the first linker and second linker each consist of 3, 4, 5, 6, 7, 8, 9, 10, 11, 12 or 13 amino acids. In some embodiments, the first linker and second linker each consist of 3, 4, 5, 6, 7, 8, 9, 10, 11, 12, 13, 14, 15, 16, 17, 18 or 19 amino acids. In some embodiments, the first linker and second linker each consists of 10 amino acids. In some embodiments, the first linker and second linker each consists of (GGGGS)_{N} (SEQ ID NO: 1), wherein _{N}=1-3. In some embodiments, the second ABR is attached, directly or indirectly, to the N-terminus of the second polypeptide. In some embodiments, the second ABR is attached, directly or indirectly, to the N-terminus of the third polypeptide. In some embodiments, _{N}=2.

In some embodiments, the VH domain of the first ABR interacts with the VL domain of the first ABR. In some embodiments, the VH domain of the second ABR interacts with the VL domain of the second ABR. In some embodiments, at least one variable domain of the first ABR interacts with at least one variable domain of the second ABR. In some embodiments, the VH domain of the first ABR interacts with the VL domain of the second ABR. In some embodiments, the VL domain of the first ABR interacts with the VH domain of the second ABR. In some embodiments, the VL domain of the first ABR interacts with the VH domain of the second ABR and wherein the VH domain of the first ABR interacts with the VL domain of the second ABR. In some embodiments, the interaction of the VL domain of the first ABR with the VH domain of the second ABR and the interaction of the VH domain of the first ABR with the VL domain of the second ABR results in a circularized conformation.

In some embodiments, the VH domain of the first ABR comprises a cysteine at amino acid residue 44 of the VH domain according to the Kabat numbering system and wherein the VL domain of the first ABR comprises a cysteine residue at amino acid residue 100 of the VL domain according to the Kabat numbering system. In some embodiments, the VH domain of the second ABR comprises a cysteine at amino acid residue 44 of the VH domain according to the Kabat numbering system and wherein the VL domain of the second ABR comprises a cysteine residue at amino acid residue 100 of the VL domain according to the Kabat numbering system. In some embodiments, the VH domain of the second ABR comprises a cysteine at amino acid residue 44 of the VH domain according to the Kabat numbering system and wherein the VL domain of the second ABR comprises a cysteine residue at amino acid residue 100 of the VL domain according to the Kabat numbering system. In some embodiments, the VH domain of the first ABR comprises a cysteine at amino acid residue 44 of the VH domain according to the Kabat numbering system and wherein the VL domain of the first ABR comprises a cysteine residue at amino acid residue 100 of the VL domain according to the Kabat numbering system; and wherein the VH domain of the second ABR comprises a cysteine at amino acid residue 44 of the VH domain according to the Kabat numbering system and wherein the VL domain of the second ABR comprises a cysteine residue at amino acid residue 100 of the VL domain according to the Kabat numbering system.

In some embodiments, proteolysis of a purified population of the multispecific ABP with a cysteine protease that digests human IgG1 at one specific site in the upper hinge (KSCDKT/HTCPPC (SEQ ID NO: 2)) produces a fragment comprising the first ABR, the second ABR, and the Fab. In some embodiments, the fragment comprising the first ABR, the second ABR, and the Fab binds to Protein A and exhibits a retention time that aligns with retention time of the multispecific ABP which has not been digested with the cysteine protease, as measured by SEC-HPLC. In some embodiments, the first ABR and second ABR bind to an HLA-peptide target with a dissociation constant (K_{D}) less than or equal to 100 nM, as measured by biolayer interferometry (BLI). In some embodiments, the ABP binds to HLA-peptide targets on cells at a higher affinity than a reference ABP. In some embodiments, the ABP binds to HLA-peptide targets on cells at the same affinity as a reference ABP or at a lower affinity than a reference ABP. In some embodiments, the Fab binds to a CD3 target on an effector cell with a dissociation constant (K_{D}) less than or equal to 100 nM, as measured by EC50 of cell binding. In some embodiments, the ABP binds to CD3 targets on an effector cell at a higher affinity than a reference ABP. In some embodiments, the effector cell is a T cell or NK cell. In some embodiments, contacting the ABP with cancer cells results in at least 50%, 60%, 70%, 80%, 90% or 95% cytotoxicity. In some embodiments, the concentration of ABP is less than 1 nM. In some embodiments, the cancer cells express HLA-peptide. In some embodiments, contacting the ABP with cancer cells results in greater cytotoxicity than a reference ABP. In some embodiments, the cancer cells are A375 cells or LN229 cells. In some embodiments, the ABP results in a stable and homogenous therapeutic. In some embodiments, the ABP results in a stable and homogenous therapeutic.

In another aspect, provided herein is an isolated multispecific antigen binding protein (ABP) comprising a first antigen binding region (ABR) and a second ABR that each specifically bind a first target antigen, a Fab that specifically binds an additional target antigen that is distinct from the first target antigen, and an Fc domain, wherein the ABP comprises a first polypeptide, a second polypeptide, and a third polypeptide, wherein the first polypeptide comprises, in an N→C direction, the first ABR-a first hinge-CH2-CH3, wherein the second polypeptide comprises, in an N→C direction, a VH domain of the Fab-a CH1 domain of the Fab-a second hinge-CH2-CH3, wherein the third polypeptide comprises, in an N→C direction, a VL domain of the Fab-a CL domain of the Fab; wherein the second ABR is attached, directly or indirectly, to the N-terminus of the second polypeptide or the third polypeptide; wherein the first ABR and second ABR each comprise, in an N→C direction: (i) a VH domain-a VL domain or (ii) a VL domain-VH domain; wherein the VH domain of the first ABR is attached to the VL domain of the first ABR via a first linker; wherein the VH domain of the second ABR is attached to the VL domain of the second ABR via a second linker; wherein the first linker and the second linker each comprise 14 amino acids; wherein (i) the VH domain of the first ABR comprises a cysteine at amino acid residue 44 of the VH domain according to the Kabat numbering system and wherein the VL domain of the first ABR comprises a cysteine residue at amino acid residue 100 of the VL domain according to the Kabat numbering system or (ii) wherein the VH domain of the second ABR comprises a cysteine at amino acid residue 44 of the VH domain according to the Kabat numbering system and wherein the VL domain of the second ABR comprises a cysteine residue at amino acid residue 100 of the VL domain according to the Kabat numbering system.

In some embodiments, the VH domain of the first ABR comprises a cysteine at amino acid residue 44 of the VH domain according to the Kabat numbering system and wherein the VL domain of the first ABR comprises a cysteine residue at amino acid residue 100 of the VL domain according to the Kabat numbering system; and wherein the VH domain of the second ABR comprises a cysteine at amino acid residue 44 of the VH domain according to the Kabat numbering system and wherein the VL domain of the second ABR comprises a cysteine residue at amino acid residue 100 of the VL domain according to the Kabat numbering system. In some embodiments, the first ABR is attached to the hinge in the first polypeptide via a third linker; wherein the second ABR is attached to the N-terminus of the second polypeptide or the third polypeptide via a fourth linker; and wherein the third linker and the fourth linker are each 30 amino acids or less in length. In some embodiments, the first linker and the second linker each consist of 20, 21, 22, 23, 24, 25, 26, 27, 28, 29 or 30 amino acids. In some embodiments, the third linker and the fourth linker each consist of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids. In some embodiments, the third linker and the fourth linker each consist of 5-15 amino acids. In some embodiments, the third linker and the fourth linker each consist of 10 amino acids. In some embodiments, the first linker and the second linker each consist of (GGGGS)_{N} (SEQ ID NO: 3), wherein _{N}=4. In some embodiments, the third linker and the fourth linker each consist of (GGGGS)_{X} (SEQ ID NO: 4), wherein _{X}=².

In some embodiments, proteolysis of a purified population of the multispecific ABP with a cysteine protease that digests human IgG1 at one specific site in the upper hinge (KSCDKT/HTCPPC (SEQ ID NO: 2)) produces (i) a first fragment comprising the first ABR and the Fc domain, and (ii) a second fragment comprising the second ABR and the Fab. In some embodiments, the first fragment binds to Protein A and exhibits a retention time that is greater than retention time of the multispecific ABP which has not been digested with the cysteine protease, as measured by SEC-HPLC. In some embodiments, the second fragment does not bind to Protein A and exhibits a retention time that is greater than retention time of the multispecific ABP which has not been digested with the cysteine protease, as measured by SEC-HPLC. In some embodiments, proteolysis of a purified population of the multispecific ABP with a cysteine protease that digests human IgG1 at one specific site in the upper hinge (KSCDKT/HTCPPC (SEQ ID NO: 2)) does not produce a fragment comprising the first ABR, the second ABR, and the Fab. In some embodiments, the proteolysis does not result in a fragment exhibiting a retention time that aligns with retention time of the multispecific ABP which has not been digested with the cysteine protease, as measured by SEC-HPLC. In some embodiments, the first ABR and second ABR bind to an HLA-peptide target with a dissociation constant (K_{D}) less than or equal to 100 nM, as measured by biolayer interferometry (BLI). In some embodiments, the ABP binds to HLA-peptide targets on cells at a higher affinity than a reference ABP. In some embodiments, the Fab binds to a CD3 target on an effector cell with a dissociation constant (K_{D}) less than or equal to 100 nM, as measured by EC50 by FACS. In some embodiments, the ABP binds to CD3 targets on an effector cell at a higher affinity than a reference ABP. In some embodiments, the ABP binds to CD3 targets on an effector cell at the same affinity as a reference ABP or at a lower affinity than the reference ABP. In some embodiments, the effector cell is a T cell or NK cell. In some embodiments, contacting the ABP with cancer cells results in at least 50%, 60%, 70%, 80%, 90% or 95% cytotoxicity. In some embodiments, the concentration of ABP is less than 1 nM. In some embodiments, the cancer cells express HLA-peptide. In some embodiments, contacting the ABP with cancer cells results in greater cytotoxicity than a reference ABP. In some embodiments, the cancer cells are A375 cells or LN229 cells. In some embodiments, the ABP results in a stable and homogenous therapeutic.

In some aspects of the present disclosure, the Fab binds to an HLA-PEPTIDE target, wherein the HLA-PEPTIDE target comprises an HLA-restricted peptide complexed with an HLA Class I molecule, wherein the HLA-restricted peptide is located in the peptide binding groove of an α1/α2 portion of the HLA Class I molecule, and wherein the HLA-PEPTIDE target is selected from Table A, Table A1, or Table A2.

In some embodiments, the first ABR or the second ABR bind to an HLA-PEPTIDE target, wherein the HLA-PEPTIDE target comprises an HLA-restricted peptide complexed with an HLA Class I molecule, wherein the HLA-restricted peptide is located in the peptide binding groove of an α1/α2 portion of the HLA Class I molecule, and wherein the HLA-PEPTIDE target is selected from Table A, Table A1, or Table A2.

In some embodiments, either the first ABR or the second ABR binds to an additional target antigen that is not an HLA-Peptide target. In some embodiments, either the first ABR and the second ABR binds to an additional target antigen.

The first ABR, second ABR, and Fab can be referred to as three different binders. In some of such embodiments, one of the binders binds to CD3. In some embodiments, the other two binders bind to an HLA-PEPTIDE target (different or the same). In alternate embodiments, one binder binds to CD3, a second binder binds to an HLA-peptide target, and a third binder binds to a receptor on a effector cells (e.g., T cell) that is not CD3, for example, CD28. In some embodiments, where two binders bind to a receptor on an effector cell (e.g., T cell), one but not both binders will bind CD3. Without being bound by theory or mechanism, this is to avoid overstimulation of the T cells.

In some embodiments, the first ABR and the second ABR each bind to an HLA-PEPTIDE target, wherein the HLA-PEPTIDE target comprises an HLA-restricted peptide complexed with an HLA Class I molecule, wherein the HLA-restricted peptide is located in the peptide binding groove of an α1/α2 portion of the HLA Class I molecule, and wherein the HLA-PEPTIDE target is selected from Table A, Table A1, or Table A2. In some embodiments:
a. the HLA Class I molecule is HLA subtype A*01:01 and the HLA-restricted peptide comprises the sequence NTDNNLAVY (SEQ ID NO: 5),
b. the HLA Class I molecule is HLA subtype A*02:01 and the HLA-restricted peptide comprises the sequence AIFPGAVPAA (SEQ ID NO: 6);
c. the HLA Class I molecule is HLA subtype A*01:01 and the HLA-restricted peptide comprises the sequence ASSLPTTMNY (SEQ ID NO: 7);
d. the HLA Class I molecule is HLA subtype A*02:01 and the HLA-restricted peptide comprises the sequence LLASSILCA (SEQ ID NO: 8); or
e. the HLA Class I molecule is HLA subtype B*35:01 and the HLA-restricted peptide comprises the sequence EVDPIGHVY (SEQ ID NO: 9).

In some embodiments, the HLA-restricted peptide is between about 5-15 amino acids in length. In some embodiments, the HLA-restricted peptide is between about 8-12 amino acids in length. In some embodiments, the additional target antigen is a cell surface molecule present on an effector cell. In some embodiments, the effector cell is a T cell. In some embodiments, the cell surface molecule is CD3, optionally CD3R. In some embodiments, the cell surface molecule is CD28. In some embodiments, the effector cell is an NK cell. In some embodiments, the cell surface molecule is CD16.

In some embodiments, the ABP further comprises an engineered disulfide bridge between the third linker and fourth linker. In some embodiments, the ABP comprises a third linker (L3) and fourth linker (L4) selected from a construct in Table 39, and wherein the L3 and L4 are from the same construct in Table 40.

In some embodiments, a sequence comprising the CH2-CH3 domains of the first polypeptide is distinct from a sequence comprising the CH2-CH3 domains of the second polypeptide. In some embodiments, the CH2-CH3 domains of the first polypeptide and/or the CH2-CH3 domains of the second polypeptide comprise a variant CH2-CH3 domain. In some embodiments, the variant CH2-CH3 domain comprises a modification that alters an affinity of the ABP for an Fc receptor as compared to a multispecific ABP with a non-variant CH2-CH3 domain. In some embodiments, the first hinge comprises a C220S mutation, according to EU numbering. In some embodiments, the variant CH2-CH3 domain comprises a human IgG4 Fc region comprising one or more of the hinge stabilizing mutations S228P and L235E, or comprising one or more of the following mutations: E233P, F234V, and L235A, according to EU numbering. In some embodiments, the variant CH2-CH3 domain is a human IgG1 Fc region comprising one or more mutations to reduce Fc receptor binding, optionally wherein the one or more mutations are in residues selected from S228 (e.g., S228A), L234 (e.g., L234A), L235 (e.g., L235A), D265 (e.g., D265A), and N297 (e.g., N297A or N297Q), or optionally wherein the amino acid sequence ELLG (SEQ ID NO: 10), from amino acid position 233 to 236 of IgG1 or EFLG (SEQ ID NO: 11) of IgG4, is replaced by PVA, according to EU numbering. In some embodiments, the variant CH2-CH3 domain is a human IgG2 Fc region comprising one or more of mutations A330S and P331S, according to EU numbering. In some embodiments, the variant CH2-CH3 domain comprises an amino acid substitution at one or more positions selected from 238, 265, 269, 270, 297, 327 and 329, optionally wherein the variant CH2-CH3 domain comprises substitutions at two or more of amino acid positions 265, 269, 270, 297 and 327, optionally wherein the variant CH2-CH3 domain comprises substitution of residues 265 or 297 with alanine, optionally wherein the variant CH2-CH3 domain comprises substitution of residues 265 and 297 with alanine, according to EU numbering.

In some embodiments, the variant CH2-CH3 domain comprises one or more amino acid substitutions that reduce at least one Fc effector function. In some embodiments, the variant CH2-CH3 domain comprises one or more amino acid substitutions that reduce binding to an Fc receptor on the cell surface of an effector cell. In some embodiments, the Fc receptor on the cell surface of an effector cell is selected from: FcγRI; FcγRIIA; FcγRIIB1; FcγRIIIB2; FcγRIIIA; and FcγRIIIB receptors. In some embodiments, the one or more amino acid substitutions is selected from: L234, L235, P331, L234F, L235E, and P331S, according to the EU numbering system. In some embodiments, the variant CH2-CH3 domain comprises the amino acid substitutions of L234F, L235E, and P331S, according to the EU numbering system. In some embodiments, the Fc effector function that is reduced comprises one or more functions selected from: complement-dependent cytotoxicity (CDC), antibody-dependent cellular cytotoxicity (ADCC), and complement fixation.

In some embodiments, the variant CH2-CH3 domain comprises one or more amino acid substitutions which improve ADCC, such as a substitution at one or more of positions 298, 333, and 334 of the variant CH2-CH3 domain, or a substitution at one or more of positions 239, 332, and 330 of the variant CH2-CH3 domain, according to EU numbering. In some embodiments, the variant CH2-CH3 domain comprises one or more modifications to increase half-life, optionally wherein the variant CH2-CH3 domain comprises substitutions at one or more of the variant CH2-CH3 domain residues: 238, 250, 256, 265, 272, 286, 303, 305, 307, 311, 312, 314, 317, 340, 356, 360, 362, 376, 378, 380, 382, 413, 424, 428, and 434 of an IgG, according to EU numbering. In some embodiments, the ABP comprises a G1m17,1 allotype.

In some embodiments, the variant CH2-CH3 domain of the first polypeptide comprises a knob-in-hole modification. In some embodiments, the variant CH2-CH3 domain of the second polypeptide comprises a knob-in-hole modification. In some embodiments, one CH2-CH3 domain-bearing chain of the multispecific ABP comprises a T366W mutation, and the other CH2-CH3 domain-bearing chain of the multispecific ABP comprises a T366S, L368A, and Y407V mutation, according to EU numbering.

In some embodiments, the ABP further comprises an engineered disulfide bridge between the CH2-CH3 domains. In some embodiments:
a. the engineered disulfide bridge comprises a K392C mutation in one CH2-CH3 domain of the multispecific ABP, and a D399C in the other CH2-CH3 domain of the multispecific ABP, according to EU numbering,
b. the engineered disulfide bridge comprises a S354C mutation in one CH2-CH3 domain of the multispecific ABP, and a Y349C mutation in the other CH2-CH3 domain of the multispecific ABP, according to EU numbering, or
c. the engineered disulfide bridge comprises a 447C mutation in both CH2-CH3 domains of the multispecific ABP, which 447C mutations are provided by extension of the C-terminus of a CH3 domain incorporating a KSC tripeptide sequence, according to EU numbering.

In some embodiments, the ABP comprises an S354C and T366W mutation in one CH2-CH3 domain and a Y349C, T366S, L368A and Y407V mutation in the other CH2-CH3 domain, according to EU numbering.

In some embodiments, one of the variant CH2-CH3 domains is capable of binding Protein A and the other variant CH2-CH3 domain comprises a mutation that reduces binding affinity of such CH2-CH3 domain to Protein A as compared to the first CH2-CH3 domain. In some embodiments, the other CH2-CH3 domain comprises a H435, Y436, H435R, Y436F, or H435R_Y436F mutation, according to EU numbering.

In some embodiments:
a. one of the variant CH2-CH3 domains comprises a F405A and a Y407V mutation and the other variant CH2-CH3 domain comprises a T394W mutation,
b. one of the variant CH2-CH3 domains comprises a F405A and a Y407V mutation and the other variant CH2-CH3 domain comprises a T366I and a T394W mutation,
c. a one of the variant CH2-CH3 domains comprises a F405A and a Y407V mutation and the other variant CH2-CH3 domain comprises a T366L and a T394W mutation,
d. one of the variant CH2-CH3 domains comprises a F405A and a Y407V mutation and the other variant CH2-CH3 domain comprises a T366L mutation, a K392M mutation, and a T394W mutation,
e. one of the variant CH2-CH3 domains comprises a L351Y mutation, a F405A mutation, and a Y407V mutation and the other variant CH2-CH3 domain comprises a T366L mutation, a K392M mutation, and a T394W mutation,
f. one of the variant CH2-CH3 domains comprises a T350V mutation, a L351Y mutation, a F405A mutation, and a Y407V mutation and the other variant CH2-CH3 domain comprises a T350V mutation, a T366L mutation, a K392M mutation, and a T394W mutation, or
g. one of the variant CH2-CH3 domains comprises a T350V mutation, a L351Y mutation, a F405A mutation, and a Y407V mutation and the other variant CH2-CH3 domain comprises a T350V mutation, a T366L mutation, a K392M mutation, and a T394W mutation,
   according to EU numbering.

In some embodiments, the variant CH2-CH3 domain is an IgG1 Fc comprising a K409R mutation in one CH2-CH3 domain and a mutation selected from a Y407, L368, F405, K370, and D399 mutation in the CH2-CH3 domain, according to EU numbering. In some embodiments, the variant CH2-CH3 domain comprises a set of mutations that renders homodimerization electrostatically unfavorable but heterodimerization favorable. In some embodiments, the variant CH2-CH3 domain comprises a K409D and a K392D mutation in one CH2-CH3 domain, and a D399K and a E356K mutation in the other CH2-CH3 domain, according to EU numbering. In some embodiments, the variant CH2-CH3 domain comprises a K409R mutation in one CH2-CH3 domain and a L368E or L368D mutation in the other CH2-CH3 domain, according to EU numbering. In some embodiments, the variant CH2-CH3 domain comprises a D221E, P228E, and L368E mutation in one the variant CH2-CH3 domain and a D221R, P228R, and K409R in the other CH2-CH3 domain, according to EU numbering. In some embodiments, the variant CH2-CH3 domain comprises an S364H and F405A mutation in one CH2-CH3 domain and a Y349T and T394F mutation in the other CH2-CH3 domain, according to EU numbering. In some embodiments, the variant CH2-CH3 domain comprises an E375Q and S364K mutation in one CH2-CH3 domain and a L368D and K370S mutation in the other CH2-CH3 domain, according to EU numbering. In some embodiments, the variant CH2-CH3 domain comprises strand-exchange engineered domain (SEED) CH3 heterodimers.

In some embodiments, the HLA Class I molecule is HLA subtype A*01:01 and the HLA-restricted peptide comprises the sequence NTDNNLAVY (SEQ ID NO: 5). In some embodiments, the HLA Class I molecule is HLA subtype A*01:01 and the HLA-restricted peptide consists of the sequence NTDNNLAVY (SEQ ID NO: 5). In some embodiments, the ABP comprises a CDR-H3 comprising a sequence selected from: CAATEWLGVW (SEQ ID NO: 12), CARANWLDYW (SEQ ID NO: 13), CARANWLDYW (SEQ ID NO: 13), CARDWVLDYW (SEQ ID NO: 14), CARGEWLDYW (SEQ ID NO: 15), CARGWELGYW (SEQ ID NO: 16), CARDFVGYDDW (SEQ ID NO: 17), CARDYGDLDYW (SEQ ID NO: 18), CARGSYGMDVW (SEQ ID NO: 19), CARDGYSGLDVW (SEQ ID NO: 20), CARDSGVGMDVW (SEQ ID NO: 21), CARDGVAVASDYW (SEQ ID NO: 22), CARGVNVDDFDYW (SEQ ID NO: 23), CARGDYTGNWYFDLW (SEQ ID NO: 24), CARANWLDYW (SEQ ID NO: 13), CARDQFYGGNSGGHDYW (SEQ ID NO: 25), CAREEDYW (SEQ ID NO: 26), CARGDWFDPW (SEQ ID NO: 27), CARGDWFDPW (SEQ ID NO: 27), CARGEWFDPW (SEQ ID NO: 28), CARSDWFDPW (SEQ ID NO: 29), CARDSGSYFDYW (SEQ ID NO: 30), CARDYGGYVDYW (SEQ ID NO: 31), CAREGPAALDVW (SEQ ID NO: 32), CARERRSGMDVW (SEQ ID NO: 33), CARVLQEGMDVW (SEQ ID NO: 34), CASERELPFDIW (SEQ ID NO: 35), CAKGGGGYGMDVW (SEQ ID NO: 36), CAAMGIAVAGGMDVW (SEQ ID NO: 37), CARNWNLDYW (SEQ ID NO: 38), CATYDDGMDVW (SEQ ID NO: 39), CARGGGGALDYW (SEQ ID NO: 40), CALSGNYYGMDVW (SEQ ID NO: 41), CARGNPWELRLDYW (SEQ ID NO: 42), and CARDKNYYGMDVW (SEQ ID NO: 43). In some embodiments, the ABP comprises a CDR-L3 comprising a sequence selected from: CQQSYNTPYTF (SEQ ID NO: 44), CQQSYSTPYTF (SEQ ID NO: 45), CQQSYSTPYSF (SEQ ID NO: 46), CQQSYSTPFTF (SEQ ID NO: 47), CQQSYGVPYTF (SEQ ID NO: 48), CQQSYSAPYTF (SEQ ID NO: 49), CQQSYSAPYTF (SEQ ID NO: 49), CQQSYSAPYSF (SEQ ID NO: 50), CQQSYSTPYTF (SEQ ID NO: 45), CQQSYSVPYSF (SEQ ID NO: 51), CQQSYSAPYTF (SEQ ID NO: 49), CQQSYSVPYSF (SEQ ID NO: 51), CQQSYSTPQTF (SEQ ID NO: 52), CQQLDSYPFTF (SEQ ID NO: 53), CQQSYSSPYTF (SEQ ID NO: 54), CQQSYSTPLTF (SEQ ID NO: 55), CQQSYSTPYSF (SEQ ID NO: 46), CQQSYSTPYTF (SEQ ID NO: 45), CQQSYSTPYTF (SEQ ID NO: 45), CQQSYSTPFTF (SEQ ID NO: 47), CQQSYSTPTF (SEQ ID NO: 56), CQQTYAIPLTF (SEQ ID NO: 57), CQQSYSTPYTF (SEQ ID NO: 45), CQQSYIAPFTF (SEQ ID NO: 58), CQQSYSIPLTF (SEQ ID NO: 59), CQQSYSNPTF (SEQ ID NO: 60), CQQSYSTPYSF (SEQ ID NO: 46), CQQSYSDQWTF (SEQ ID NO: 61), CQQSYLPPYSF (SEQ ID NO: 62), CQQSYSSPYTF (SEQ ID NO: 54), CQQSYTTPWTF (SEQ ID NO: 63), CQQSYLPPYSF (SEQ ID NO: 62), CQEGITYTF (SEQ ID NO: 64), CQQYYSYPFTF (SEQ ID NO: 65), and CQHYGYSPVTF (SEQ ID NO: 66).

In some embodiments, the ABP comprises the CDR-H3 and the CDR-L3 from the scFv designated G2(1H11), G2(2E07), G2(2E03), G2(2A11), G2(2C06), G2(1G01), G2(1C02), G2(1H01), G2(1B12), G2(1B06), G2(2H10), G2(1H10), G2(2C11), G2(1C09), G2(1A10), G2(1B10), G2(1D07), G2(1E05), G2(1D03), G2(1G12), G2(2H11), G2(1C03), G2(1G07), G2(1F12), G2(1G03), G2(2B08), G2(2A10), G2(2D04), G2(1C06), G2(2A09), G2(1B08), G2(1E03), G2(2A03), G2(2F01), or G2(1D06). In some embodiments, the ABP comprises the CDR-H3 and the CDR-L3 from the scFv designated G2(1H11) or G2(2C11). In some embodiments, the ABP comprises the CDR-H3 and the CDR-L3 from the scFv designated G2(2E07)

In some embodiments, the ABP comprises all three heavy chain CDRs (CDR-H1, CDR-H2, CDR-H3) and all three light chain CDRs (CDR-L1, CDR-L2, CDR-L3) from the scFv designated G2(1H11), G2(2E07), G2(2E03), G2(2A11), G2(2C06), G2(1G01), G2(1C02), G2(1H01), G2(1B12), G2(1B06), G2(2H10), G2(1H10), G2(2C11), G2(1C09), G2(1A10), G2(1B10), G2(1D07), G2(1E05), G2(1D03), G2(1G12), G2(2H11), G2(1C03), G2(1G07), G2(1F12), G2(1G03), G2(2B08), G2(2A10), G2(2D04), G2(1C06), G2(2A09), G2(1B08), G2(1E03), G2(2A03), G2(2F01), or G2(1D06). In some embodiments, the ABP comprises all three heavy chain CDRs and all three light chain CDRs from the scFv designated G2(1H11). In some embodiments, the ABP comprises all three heavy chain CDRs and all three light chain CDRs from the scFv designated G2(2E07).

In some embodiments, the ABP comprises a VH sequence selected from Table 27. In some embodiments, the ABP comprises a VL sequence selected from Table 27. In some embodiments, the ABP comprises a VH sequence and VL sequence selected from Table 27, wherein the VH sequence and VL sequence are selected from the same clone. In some embodiments, the ABP comprises the VH sequence and the VL sequence are from the scFv designated G2(2E07).

In some embodiments, the ABP comprises the VH sequence and the VL sequence are from the scFv designated G2(1H11). In some embodiments, the multispecific ABP binds to any one or more of amino acid positions 3-9 of the restricted peptide NTDNNLAVY (SEQ ID NO: 5). In some embodiments, the multispecific ABP binds to any one or more of amino acid positions 6-9 of the restricted peptide NTDNNLAVY (SEQ ID NO: 5). In some embodiments, the multispecific ABP binds to any one or more of amino acid positions 70-85 of the alpha 1 helix of HLA subtype A*01:01. In some embodiments, the multispecific ABP binds to any one or more of amino acid positions 140-160 of the alpha 2 helix of HLA subtype A*01:01. In some embodiments, the multispecific ABP binds to any one or more of amino acid positions 157-160 of the alpha 2 helix of HLA subtype A*01:01.

In some embodiments, the HLA Class I molecule is HLA subtype B*35:01 and the HLA-restricted peptide comprises the sequence EVDPIGHVY (SEQ ID NO: 9). In some embodiments, the HLA Class I molecule is HLA subtype B*35:01 and the HLA-restricted peptide consists of the sequence EVDPIGHVY (SEQ ID NO: 9). In some embodiments, the ABP comprises a CDR-H3 comprising a sequence selected from: CARDGVRYYGMDVW (SEQ ID NO: 67), CARGVRGYDRSAGYW (SEQ ID NO: 68), CASHDYGDYGEYFQHW (SEQ ID NO: 69), CARVSWYCSSTSCGVNWFDPW (SEQ ID NO: 70), CAKVNWNDGPYFDYW (SEQ ID NO: 71), CATPTNSGYYGPYYYYGMDVW (SEQ ID NO: 72), CARDVMDVW (SEQ ID NO: 73), CAREGYGMDVW (SEQ ID NO: 74), CARDNGVGVDYW (SEQ ID NO: 75), CARGIADSGSYYGNGRDYYYGMDVW (SEQ ID NO: 76), CARGDYYFDYW (SEQ ID NO: 77), CARDGTRYYGMDVW (SEQ ID NO: 78), CARDVVANFDYW (SEQ ID NO: 79), CARGHSSGWYYYYGMDVW (SEQ ID NO: 80), CAKDLGSYGGYYW (SEQ ID NO: 81), CARSWFGGFNYHYYGMDVW (SEQ ID NO: 82), CARELPIGYGMDVW (SEQ ID NO: 83), and CARGGSYYYYGMDVW (SEQ ID NO: 84). In some embodiments, the ABP comprises a CDR-L3 comprising a sequence selected from: CMQGLQTPITF (SEQ ID NO: 85), CMQALQTPPTF (SEQ ID NO: 86), CQQAISFPLTF (SEQ ID NO: 87), CQQANSFPLTF (SEQ ID NO: 88), CQQANSFPLTF (SEQ ID NO: 88), CQQSYSIPLTF (SEQ ID NO: 59), CQQTYMMPYTF (SEQ ID NO: 89), CQQSYITPWTF (SEQ ID NO: 90), CQQSYITPYTF (SEQ ID NO: 91), CQQYYTTPYTF (SEQ ID NO: 92), CQQSYSTPLTF (SEQ ID NO: 55), CMQALQTPLTF (SEQ ID NO: 93), CQQYGSWPRTF (SEQ ID NO: 94), CQQSYSTPVTF (SEQ ID NO: 95), CMQALQTPYTF (SEQ ID NO: 96), CQQANSFPFTF (SEQ ID NO: 97), CMQALQTPLTF (SEQ ID NO: 93), and CQQSYSTPLTF (SEQ ID NO: 55).

In some embodiments, the ABP comprises the CDR-H3 and the CDR-L3 from the scFv designated G5(7A05), G5(1C12), G5(7E07), G5(7B03), G5(7F06), G5(1B12), G5(1E05), G5(3G01), G5(3G08), G5(4B02), G5(4E04), G5(1D06), G5(1H11), G5(2B10), G5(2H08), G5(3G05), G5(4A07), or G5(4B01). In some embodiments, the ABP comprises the CDR-H3 and the CDR-L3 from the scFv designated G5(1C12) or G5(1H11). In some embodiments, the ABP comprises all three heavy chain CDRs and all three light chain CDRs from the scFv designated G5(7A05), G5(1C12), G5(7E07), G5(7B03), G5(7F06), G5(1B12), G5(1E05), G5(3G01), G5(3G08), G5(4B02), G5(4E04), G5(1D06), G5(1H11), G5(2B10), G5(2H08), G5(3G05), G5(4A07), or G5(4B01). In some embodiments, the ABP comprises all three heavy chain CDRs and all three light chain CDRs from the scFv designated G5(1C12) or G5(1H11).

In some embodiments, the ABP comprises a VH sequence selected from Table 4. In some embodiments, the ABP comprises a VL sequence selected from Table 4. In some embodiments, the ABP comprises a VH sequence and VL sequence selected from Table 4, wherein the VH sequence and VL sequence are selected from the same clone. In some embodiments, the ABP comprises the VH sequence and VL sequence from the scFv designated G5(1C12) or G5(1H11). In some embodiments, the ABP binds to any one or more of amino acid positions 2-8 on the restricted peptide EVDPIGHVY (SEQ ID NO: 9).

In some embodiments, the ABP binds to any one or more of amino acid positions 50, 54, 55, 57, 61, 62, 74, 81, 82 and 85 of the α1 helix of the HLA protein. In some embodiments, the ABP binds to any one or more of amino acid positions 147 and 148 of the α2 helix of the HLA protein.

In some embodiments, the HLA Class I molecule is HLA subtype A*02:01 and the HLA-restricted peptide comprises the sequence AIFPGAVPAA (SEQ ID NO: 6). In some embodiments, the HLA Class I molecule is HLA subtype A*02:01 and the HLA-restricted peptide consists of the sequence AIFPGAVPAA (SEQ ID NO: 6).

In some embodiments, the ABP comprises a CDR-H3 comprising a sequence selected from: CARDDYGDYVAYFQHW (SEQ ID NO: 177), CARDLSYYYGMDVW (SEQ ID NO: 178), CARVYDFWSVLSGFDIW (SEQ ID NO: 179), CARVEQGYDIYYYYYMDVW (SEQ ID NO: 180), CARSYDYGDYLNFDYW (SEQ ID NO: 181), CARASGSGYYYYYGMDVW (SEQ ID NO: 182), CAASTWIQPFDYW (SEQ ID NO: 183), CASNGNYYGSGSYYNYW (SEQ ID NO: 184), CARAVYYDFWSGPFDYW (SEQ ID NO: 185), CAKGGIYYGSGSYPSW (SEQ ID NO: 186), CARGLYYMDVW (SEQ ID NO: 187), CARGLYGDYFLYYGMDVW (SEQ ID NO: 188), CARGLLGFGEFLTYGMDVW (SEQ ID NO: 189), CARDRDSSWTYYYYGMDVW (SEQ ID NO: 190), CARGLYGDYFLYYGMDVW (SEQ ID NO: 188), CARGDYYDSSGYYFPVYFDYW (SEQ ID NO: 191), and CAKDPFWSGHYYYYGMDVW (SEQ ID NO: 192).

In some embodiments, the ABP comprises a CDR-L3 comprising a sequence selected from: CQQNYNSVTF (SEQ ID NO: 194), CQQSYNTPWTF (SEQ ID NO: 195), CGQSYSTPPTF (SEQ ID NO: 196), CQQSYSAPYTF (SEQ ID NO: 49), CQQSYSTPPTF (SEQ ID NO: 197), CQQSYSAPYTF (SEQ ID NO: 49), CQQHNSYPPTF (SEQ ID NO: 198), CQQYSTYPITI (SEQ ID NO: 199), CQQANSFPWTF (SEQ ID NO: 200), CQQSHSTPQTF (SEQ ID NO: 201), CQQSYSTPLTF (SEQ ID NO: 55), CQQSYSTPLTF (SEQ ID NO: 55), CQQTYSTPWTF (SEQ ID NO: 202), CQQYGSSPYTF (SEQ ID NO: 203), CQQSHSTPLTF (SEQ ID NO: 204), CQQANGFPLTF (SEQ ID NO: 205), and CQQSYSTPLTF (SEQ ID NO: 55).

In some embodiments, the ABP comprises the CDR-H3 and the CDR-L3 from the scFv designated G8(1A03), G8(1A04), G8(1A06), G8(1B03), G8(1C11), G8(1D02), G8(1H08), G8(2B05), G8(2E06), G8(2C10), G8(2E04), G8(4F05), G8(5C03), G8(5F02), G8(5G08), G8(1C01), or G8(2C11). In some embodiments, the ABP comprises the CDR-H3 and the CDR-L3 from the scFv designated G8(1B03). In some embodiments, the ABP comprises all three heavy chain CDRs and all three light chain CDRs from the scFv designated G8(1A03), G8(1A04), G8(1A06), G8(1B03), G8(1C11), G8(1D02), G8(1H08), G8(2B05), G8(2E06), G8(2C10), G8(2E04), G8(4F05), G8(5C03), G8(5F02), G8(5G08), G8(1C01), or G8(2C11). In some embodiments, the ABP comprises all three heavy chain CDRs and all three light chain CDRs from the scFv designated G8(1B03).

In some embodiments, the ABP comprises a VH sequence selected from Table 6. In some embodiments, the ABP comprises a VL sequence selected from Table 6. In some embodiments, the ABP comprises a VH sequence and VL sequence selected from Table 6, wherein the VH sequence and VL sequence are selected from the same clone. In some embodiments, the ABP comprises the VH sequence and VL sequence from the scFv designated G8(1B03).

In some embodiments, the ABP binds to any one or more of amino acid positions 5 and 6 on the restricted peptide AIFPGAVPAA (SEQ ID NO: 6). In some embodiments, the ABP binds to any one or more of amino acid positions 46, 49, 55, 61, 74, 76, 77, 78, 81 and 84 of the α1 helix of the HLA protein. In some embodiments, the ABP binds to any one or more of amino acid positions 137, 138, 145, 147, 152-157 of the α2 helix of the HLA protein.

In some embodiments, the antigen binding protein is linked to a scaffold, optionally wherein the scaffold comprises serum albumin or Fe, optionally wherein Fe is human Fe and is an IgG (IgG1, IgG2, IgG3, IgG4), an IgA (IgA1, IgA2), an IgD, an IgE, or an IgM isotype Fc. In some embodiments, the antigen binding protein is linked to a scaffold via a linker, optionally wherein the linker is a peptide linker, optionally wherein the peptide linker is a hinge region of a human antibody. In some embodiments, the antigen binding protein comprises one or more antibody complementarity determining regions (CDRs), optionally six antibody CDRs.

In some embodiments, the antigen binding protein is a monoclonal antibody. In some embodiments, the antigen binding protein is a humanized, human, or chimeric antibody. In some embodiments, the antigen binding protein is bispecific.

In some embodiments, the antigen binding protein comprises an isotype of a class selected from IgG, IgA, IgD, IgE, and IgM. In some embodiments, the ABP comprises an isotype of the class human IgG and a subclass selected from IgG1, IgG4, IgG2, and IgG3.

In some embodiments, the ABP comprises an isotype of the class human IgG and a subclass of IgG1. In some embodiments, the ABP comprises an isotype of the class human IgG and a subclass of IgG4. In some embodiments, the ABP comprises an isotype of the class human IgG and a subclass of IgG2. In some embodiments, the ABP comprises an isotype of the class human IgG and a subclass of IgG3.

In some embodiments, the ABP comprises a modification that extends half-life. In some embodiments, the ABP comprises a modified Fe, optionally wherein the modified Fc comprises one or more mutations that extend half-life, optionally wherein the one or more mutations that extend half-life is YTE.

In some embodiments, the antigen binding protein is a portion of a chimeric antigen receptor (CAR) comprising: an extracellular portion comprising the antigen binding protein and an intracellular signaling domain. In some embodiments, the extracellular portion comprises an scFv and the intracellular signaling domain comprises an ITAM. In some embodiments, the intracellular signaling domain comprises a signaling domain of a zeta chain of a CD3-zeta (CD3) chain. In some embodiments, the ABP further comprises a transmembrane domain linking the extracellular domain and the intracellular signaling domain. In some embodiments, the transmembrane domain comprises a transmembrane portion of CD28. In some embodiments, the ABP comprises an intracellular signaling domain of a T cell costimulatory molecule. In some embodiments, the T cell costimulatory molecule is CD28, 4-1BB, OX-40, ICOS, or any combination thereof.

In some embodiments, the antigen binding protein binds to the HLA-PEPTIDE target through a contact point with the HLA Class I molecule and through a contact point with the HLA-restricted peptide of the HLA-PEPTIDE target. In some embodiments, the contact points are determined via positional scanning, hydrogen-deuterium exchange, or protein crystallography.

In some embodiments, the ABP has one or more of the fragments as indicated in Table 54. For example, in some embodiments the ABP comprises the fragments in Chain 1 of Table 54 and the CH2-CH3 fragments in Chain 2 of Table 54.

In some embodiments, the ABP is used as a medicament. In some embodiments, the ABP is for use in treatment of cancer, optionally wherein the cancer expresses or is predicted to express the HLA-PEPTIDE target. In some embodiments, the ABP is for use in treatment of cancer, wherein the cancer is selected from a solid tumor and a hematological tumor. In some embodiments, the ABP is for use in treatment of chronic viral disease.

In another aspect, provided herein is an antigen binding protein (ABP) which is a conservatively modified variant of a multispecific ABP described herein.

In another aspect, provided herein is an antigen binding protein (ABP) that competes for binding with a multispecific ABP described herein.

In another aspect, provided herein is an antigen binding protein (ABP) that binds the same HLA-PEPTIDE epitope bound by a multispecific ABP described herein.

In another aspect, provided herein is an engineered cell expressing a receptor comprising a multispecific ABP described herein. In some embodiments, the ABP is for the engineered cell is a T cell, optionally a cytotoxic T lymphocyte (CTL). In some embodiments, the ABP in the engineered cell, the antigen binding protein is expressed from a heterologous promoter.

In another aspect, provided herein is an isolated polynucleotide or set of polynucleotides encoding a multispecific ABP described herein or an antigen-binding portion thereof. In another aspect, provided herein is a vector or set of vectors comprising the polynucleotide or set of polynucleotides.

In another aspect, provided herein is a host cell comprising the polynucleotide or set of polynucleotides as described herein or the vector or set of vectors as described herein, optionally wherein the host cell is CHO or HEK293, or optionally wherein the host cell is a T cell.

In another aspect, provided herein is a method of producing an antigen binding protein comprising expressing the antigen binding protein with the host cell as described herein and isolating the expressed antigen binding protein.

In another aspect, provided herein is a pharmaceutical composition comprising a multispecific ABP as described herein and a pharmaceutically acceptable excipient.

In another aspect, provided herein is a method of treating cancer in a subject, comprising administering to the subject an effective amount of a multispecific ABP as described herein or a pharmaceutical composition as described herein, optionally wherein the cancer is selected from a solid tumor and a hematological tumor. In some embodiments, the cancer expresses or is predicted to express the HLA-PEPTIDE target.

In another aspect, provided herein is a method of treating chronic viral disease in a subject, comprising administering to the subject an effective amount of a multispecific ABP as described herein or a pharmaceutical composition as described herein. In some embodiments, the infected cells in the subject express or are predicted to express the HLA-PEPTIDE target.

In another aspect, provided herein is a method of treating a disease or infection in a subject, comprising administering to the subject an effective amount of a multispecific ABP described herein, wherein the disease or infection involves HLA-PEPTIDE or wherein the disease or infection comprises cells that express HLA-PEPTIDE.

In another aspect, provided herein is a kit comprising a multispecific ABP as described herein or a pharmaceutical composition as described herein and instructions for use.

In another aspect, provided herein is a virus comprising the isolated polynucleotide or set of polynucleotides as described herein. In some embodiments, the virus is a filamentous bacteriophage.

In another aspect, provided herein is a method of isolating a multispecific antibody, comprising: (a) providing (i) a mixture that comprises an ABP comprising a light chain Kappa constant domain, optionally wherein the ABP as described herein, and (ii) an anti-Kappa resin, wherein the anti-Kappa resin comprises a ligand having high specificity for a light chain Kappa constant domain, and wherein contaminants lacking a light chain Kappa constant domain do not bind the anti-Kappa resin; (b) contacting (i) and (ii) under conditions that allow for differential binding to the anti-Kappa resin as compared to at least one contaminant, in the mixture, that lacks a light chain Kappa constant domain or has a different number of light chain Kappa constant domains relative to the ABP; and (c) eluting the ABP from the anti-Kappa resin under conditions that allow for differential detachment of the ABP relative to the contaminant.

In some embodiments, the differential detachment of the ABP results from differences in avidity to the anti-Kappa resin between the ABP and the contaminant. In some embodiments, the ABP comprises no more than one light chain Kappa constant domain. In some embodiments, the anti-Kappa resin is CaptureSelect™ KappaXP Affinity Matrix, CaptureSelect™ KappaXL Affinity Matrix, or KappaSelect Affinity Matrix. In some embodiments, the anti-Kappa resin is CaptureSelect™ KappaXP Affinity Matrix. In some embodiments, the ligand comprises an anti-Kappa monoclonal antibody.

In some embodiments, the conditions that allow for differential detachment of the ABP comprises a pH gradient elution. In some embodiments, the pH gradient elution is from about 6 (starting pH) to about 3 (final pH). In some embodiments, the conditions that allow for differential detachment of the ABP comprises a salt gradient elution. In some embodiments, the salt gradient elution comprises a gradient of inorganic salt. In some embodiments, the salt gradient elution comprises a NaCl gradient. In some embodiments, the NaCl gradient comprises a gradient of about 150 mM of NaCl (starting concentration) to 50 mM of NaCl (final concentration). In some embodiments, the NaCl gradient comprises a gradient of about 200 mM of NaCl (starting concentration) to 0 mM of NaCl (final concentration). In some embodiments, the NaCl gradient comprises a gradient of about 200 mM of NaCl (starting concentration) to about 50 mM of NaCl (final concentration). In some embodiments, the NaCl gradient comprises a gradient of about 500 mM of NaCl (starting concentration) to 0 mM of NaCl (final concentration). In some embodiments, the NaCl gradient comprises a gradient of about 500 mM of NaCl (starting concentration) to about 50 mM of NaCl (final concentration).

In some embodiments, the salt gradient elution comprises a pH within the range of about 3.6-4.4. In some embodiments, the salt gradient elution comprises a pH of about 3.9. In some embodiments, the salt gradient elution comprises a pH of about 4.2. In some embodiments, the salt gradient elution comprises a pH selected from: 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 and 5.0. In some embodiments, the salt gradient elution comprises a pH selected from: 3.6, 3.7, 3.8, 3.9, 4.0, 4.1, 4.2, 4.3, and 4.4.

In some embodiments, the conditions that allow for differential detachment of the ABP comprise a salt gradient and a pH gradient. In some embodiments, the conditions that allow for differential detachment of the ABP comprise a salt gradient or a pH gradient. In some embodiments, the salt gradient comprises a gradient of inorganic salt. In some embodiments, the salt gradient elution comprises a NaCl gradient.

In some embodiments, the conditions that allow for detachment of the ABP comprise a step variation in pH level and/or a step variation in the concentration of salt. In some embodiments, the conditions that allow for differential detachment of the ABP comprise a step variation in pH level and a step variation in the concentration of salt. In some embodiments, the conditions that allow for differential detachment of the ABP comprise a step variation in pH level or a step variation in the concentration of salt. In some embodiments, the salt is an inorganic salt. In some embodiments, the salt is NaCl.

In some embodiments, the step variation in pH level comprises a step at pH 3.9. In some embodiments, the conditions that allow for differential detachment of the ABP comprise a step variation in pH level. In some embodiments, the step variation in pH level comprises a step at pH 4.2. In some embodiments, the step variation in pH level comprises a pH selected from the range of pH 3.6-4.4. In some embodiments, the step variation in pH level comprises a pH selected from: 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0. In some embodiments, the step variation in pH level comprises a pH selected from: 3.6, 3.7, 3.8, 3.9, 4.0, 4.1, 4.2, 4.3, and 4.4.

In some embodiments, the conditions that allow for differential detachment of the ABP comprise a step variation in the concentration of salt. In some embodiments, the salt is an inorganic salt. In some embodiments, the inorganic salt is NaCl.

In some embodiments, the step variation in concentration of inorganic salt comprises a salt step selected from the inorganic salt concentration range of about 200 mM NaCl to about 0 mM NaCl. In some embodiments, the step variation in concentration of inorganic salt comprises a salt step of 150 mM NaCl. In some embodiments, the step variation in concentration of inorganic salt comprises a salt step of 100 mM NaCl. In some embodiments, the step variation in concentration of inorganic salt comprises a salt step of 50 mM NaCl. In some embodiments, the step variation in concentration of inorganic salt comprises a salt step of 25 mM NaCl. In some embodiments, the step variation in concentration of inorganic salt comprises a salt step selection from: 0 mM of NaCl, 5 mM of NaCl, 10 mM of NaCl, 15 mM of NaCl, 20 mM of NaCl, 25 mM of NaCl, 30 mM of NaCl, 35 mM of NaCl, 40 mM of NaCl, 45 mM of NaCl, 50 mM of NaCl, 55 mM of NaCl, 60 mM of NaCl, 65 mM of NaCl, 70 mM of NaCl, 75 mM of NaCl, 80 mM of NaCl, 85 mM of NaCl, 90 mM of NaCl, 95 mM of NaCl, 100 mM of NaCl, 105 mM of NaCl, 110 mM of NaCl, 115 mM of NaCl, 120 mM of NaCl, 125 mM of NaCl, 130 mM of NaCl, 135 mM of NaCl, 140 mM of NaCl, 145 mM of NaCl, 150 mM of NaCl, 155 mM of NaCl, 160 mM of NaCl, 165 mM of NaCl, 170 mM of NaCl, 175 mM of NaCl, 180 mM of NaCl, 185 mM of NaCl, 190 mM of NaCl, 195 mM of NaCl, and 200 mM of NaCl.

In some embodiments, the elution is conducted using an acetate buffered elution buffer.

### BRIEF DESCRIPTION OF THE DRAWINGS

These and other features, aspects, and advantages of the present invention will become better understood with regard to the following description, and accompanying drawings, where:

FIG. 1A includes a schematic showing a format 4 antibody in dual scFv (2×scFv) conformation. FIG. 1B includes a schematic showing a format 4 antibody in diabody conformation.

FIG. 2 includes a schematic showing the equilibrium between the dual scFv (2×ScFv) conformation and diabody conformation of a Format 4 antibody. Linkers are not drawn to scale.

FIG. 3 includes a schematic showing (from left to right) Format 3, Format 4 in 2×ScFv conformation, Format 5 antibody, and Format 4 antibody in diabody conformation. Linkers are not drawn to scale.

FIG. 4 includes a negative stain electron micrograph showing the compact conformation of the diabody.

FIG. 5 includes a schematic showing the proteolytic cleaving of a specific peptide sequence by FabALACTICA® enzyme. FIG. 5 discloses SEQ ID NOS 98, 729, 98, and 729, respectively, in order of appearance (top left to bottom right).

FIG. 6A shows SEC-HPLC results from a product quality screening of antibodies using a TSKgel SuperSW mAb HTP column (top panel), where a peak tailing between 4.5-5.5 minutes suggested presence of an additional antibody moiety that either interacts more with the SEC column, or is more compacted and thus migrates slower than the main antibody conformation. FIG. 6A also shows SEC-HPLC results from a TSKgel G3000SWxl column (bottom panel) which resolved the tailing into a “split peak”.

FIG. 6B includes a schematic showing two conformations of Format 4 antibodies that may exist in solution: dual scFv and diabody conformation.

FIG. 6C includes a schematic showing modifications to Format 4 to force a stable conformation in solution.

FIG. 7A shows expected protein digestion fragments of “standard” Format 4 antibodies and a “diabody” isomer of Format 4. FIG. 7B shows SEC-HPLC results from a FabALACTICA digestion experiment, where Format 4 antibodies were treated with a cysteine protease that digests human IgG1 at one specific site in the upper hinge (KSCDKT/HTCPPC (SEQ ID NO: 2)).

FIG. 8 includes a schematic representation of the undigested Format 4 “dual scFv” conformation (left), the diabody conformation without digestion (middle), and the diabody conformation after proteolytic digestion (right).

FIG. 9 shows results from an electron microscopy study of a representative Format 4 antibody, Format 4-hOKT3-G5(1C12).

FIG. 10 includes SEC-HPLC chromatograms from a Format 4 G2(1H11) bispecific antibody with an engineered VH44/VL100 disulfide bond (top panel), and without the engineered disulfide bond (bottom panel).

FIG. 11 includes SEC-HPLC chromatograms from a Format 4 G5(1C12) bispecific antibody with an engineered VH44/VL100 disulfide bond (top panel), and without the engineered disulfide bond (bottom panel).

FIG. 12A includes overlayed SEC-HPLC chromatograms for digested Format 4 G5(1C12) bispecific antibody with an engineered VH44/VL100 disulfide bond. FIG. 12B includes reduced and non-reduced gels (CE-SDS) for digested Format 4 G5(1C12) bispecific antibody with an engineered VH44/VL100 disulfide bond.

FIG. 13A includes overlayed SEC-HPLC chromatograms for digested Format 4 G2(1H11) bispecific antibody with an engineered VH44/VL100 disulfide bond. FIG. 13B includes reduced and non-reduced gels (CE-SDS) for digested Format 4 G2(1H11) bispecific antibody with an engineered VH44/VL100 disulfide bond.

FIG. 14 includes plots showing the BLI results from representative bispecific Format 4 antibodies with and without the engineered VH44/VL100 disulfide bond.

FIG. 15 includes plots showing MSD results from representative bispecific Format 4 antibodies with and without the engineered VH44/VL100 disulfide bond.

FIG. 16 includes plots showing cell binding results from representative bispecific Format 4 antibodies with and without the engineered VH44/VL100 disulfide bond.

FIG. 17 includes plots 123 showing 2D cytotoxicity and spheroid toxicity results from a representative G5 Format 4 antibody with and without the engineered VH44/VL100 disulfide bond.

FIG. 18 includes plots showing 2D cytotoxicity and spheroid toxicity results from representative G2 Format 4 antibodies with and without the engineered VH44/VL100 disulfide bond.

FIG. 19 includes a flow chart showing exemplary proteolytic digestion methods for the ABPs.

FIG. 20 includes overlayed SEC-HPLC chromatograms and reduced and non-reduced gels (CE-SDS) from digestion of a Format 3 bispecific antibody.

FIG. 21 includes overlayed SEC-HPLC chromatograms and reduced and non-reduced gels (CE-SDS) from digestion of a Format 5 bispecific antibody.

FIG. 22 includes overlayed SEC-HPLC chromatograms and reduced and non-reduced gels (SDS-PAGE) from digestion of a Format 4 G5(1C12) ABP having a non-shortened linker.

FIG. 23 includes overlayed SEC-HPLC chromatograms and reduced and non-reduced gels (SDS-PAGE) from digestion of a Format 4 G2(1H11) ABP having a non-shortened linker.

FIG. 24 includes overlayed SEC-HPLC chromatograms and reduced and non-reduced gels (SDS-PAGE) from digestion of a Format 4 G5(1C12) ABP having shortened first and second linkers (10 amino acids long).

FIG. 25 includes overlayed SEC-HPLC chromatograms and reduced and non-reduced gels (SDS-PAGE) from digestion of a Format 4 G2(1H11) ABP having shortened first and second linkers (10 amino acids long).

FIG. 26 includes an SEC-HPLC plot and reduced and non-reduced gels (CE-SDS) from digestion of a Format 4 G2(1H11) ABP having (i) shortened first and second linkers (10 amino acids long) and (ii) engineered VH44/VL100 disulfide bond.

FIG. 27 includes plots showing the BLI results from representative bispecific Format 4 antibodies with (“DAB”) and without (2×scFv) shortened first and second linkers (10 amino acids long).

FIG. 28 shows the cell binding results from the indicated G2 and G5 Format 4 ABPs.

FIG. 29 shows the cytotoxicity results for the indicated G2 and G5 Format 4 ABPs.

FIG. 30 includes schematic of Format 4 diabody constructs with disulfide bridge stabilization outside variable domains.

FIG. 31 includes a schematic showing the differences in binding avidity of ABP (heterodimer containing a single kappa constant domain), and expected associated homodimer contaminants to an affinity resin with ligand targeting light chain Kappa constant domains in the antibody.

FIG. 32 shows the results from a purification experiment to determine resolving power of CaptureSelect Kappa XL and CaptureSelect Kappa XP affinity matrices for and ABP and associated homodimer contaminants. The elution was conducted using pH gradient elution from 6-3. The elution chromatograms and non-reducing CE-SDS gels are shown.

FIG. 33 shows the results from a purification experiment using spiked load to determine the pH range where the Format 4 ABP and associated Kappa constant domain-containing contamiants elute. The elution chromatograms, non-reducing CE-SDS gels, and SEC-HPLC chromatograms are shown.

FIG. 34 includes the chromatograms of the elution step obtained for purifications of the ABP containing a single kappa constant domain, from a load containing all expected homodimer contaminants while conducting elution NaCl gradients at pH 4.2, 3.9 or 3.6.

FIG. 35 includes the non-reducing CE-SDS gels for eluate fractions obtained from the purification experiments with elution NaCl gradient elutions conducted at the indicated pH levels, shown in FIG. 34.

FIG. 36 includes an overlay of SEC-HPLC chromatograms of pooled eluate peak 1 and pooled eluate peak peak 2 obtained upon conducting the purification experiment with NaCl gradient elution at pH 3.9, shown in FIG. 34.

FIGS. 37A and 37B include size exclusion chromatograms of modified Format 4 antibodies. The two arrows in FIG. 37A show two antibodies that stably formed diabodies. The two antibodies in FIG. 37B also stably formed diabodies.

FIGS. 38A-38B include plots showing SDS page gels from the digestion of Format 41 antibody (FIG. 38A) and Format 3 antibody, which served as a control (FIG. 38B).

### DETAILED DESCRIPTION

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise. For example, a multispecific ABP “comprising a diabody” includes a multispecific ABP “consisting of a diabody” and a multispecific ABP “consisting essentially of a diabody.”

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antigen binding protein” or “ABP” is used herein in its broadest sense and includes certain types of molecules comprising one or more antigen-binding domains that specifically bind to an antigen or epitope.

In some embodiments, the ABP comprises an antibody. In some embodiments, the ABP consists of an antibody. In some embodiments, the ABP consists essentially of an antibody. An ABP specifically includes intact antibodies (e.g., intact immunoglobulins), antibody fragments, ABP fragments, and multi-specific antibodies. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. In some embodiments, a CAR comprises an ABP provided herein. An “HLA-PEPTIDE ABP,” “anti-HLA-PEPTIDE ABP,” or “HLA-PEPTIDE-specific ABP” is an ABP, as provided herein, which specifically binds to the antigen HLA-PEPTIDE. An ABP includes proteins comprising one or more antigen-binding domains that specifically bind to an antigen or epitope via a variable region, such as a variable region derived from a B cell (e.g., antibody) or T cell (e.g., TCR).

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody, camelid VHH, engineered or evolved human VH that does not require pairing to VL for solubility or activity) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

As used herein, “variable region” refers to a variable nucleotide sequence that arises from a recombination event, for example, it can include a V, J, and/or D region of an immunoglobulin or T cell receptor (TCR) sequence from a B cell or T cell, such as an activated T cell or an activated B cell.

The term “antigen-binding domain” means the portion of an ABP that is capable of specifically binding to an antigen or epitope. One example of an antigen-binding domain is an antigen-binding domain formed by an antibody V_{H}-V_{L} dimer of an ABP. Another example of an antigen-binding domain is an antigen-binding domain formed by diversification of certain loops from the tenth fibronectin type III domain of an Adnectin. An antigen-binding domain can include antibody CDRs 1, 2, and 3 from a heavy chain in that order; and antibody CDRs 1, 2, and 3 from a light chain in that order. An antigen-binding domain can include TCR CDRs, e.g., αCDR1, αCDR2, αCDR3, PCDR1, PCDR2, and PCDR3. TCR CDRs are described herein.

The antibody VH and VL regions may be further subdivided into regions of hypervariability (“hypervariable regions (HVRs);” also called “complementarity determining regions” (CDRs)) interspersed with regions that are more conserved. The more conserved regions are called framework regions (FRs). Each VH and VL generally comprises three antibody CDRs and four FRs, arranged in the following order (from N-terminus to C-terminus): FR1-CDR1-FR2-CDR2-FR3-CDR3-FR4. The antibody CDRs are involved in antigen binding, and influence antigen specificity and binding affinity of the ABP. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called Kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of an antibody CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme); each of which is incorporated by reference in its entirety.

Table 14 provides the positions of antibody CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Antibody CDRs may be assigned, for example, using ABP numbering software, such as Abnum, available at www.bioinf.org.uk/abs/abnum/, and described in Abhinandan and Martin, Immunology, 2008, 45:3832-3839, incorporated by reference in its entirety.

TABLE 14
Residues in CDRs according to Kabat and Chothia numbering schemes

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an ABP heavy chain constant region (e.g., as reported in Kabat et al., supra). Unless stated otherwise, the EU numbering scheme is used to refer to residues in ABP heavy chain constant regions described herein.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The amino acid sequence boundaries of a TCR CDR can be determined by one of skill in the art using any of a number of known numbering schemes, including but not limited to the IMGT unique numbering, as described by LeFranc, M.-P, Immunol Today. 1997 November; 18(11):509; Lefranc, M.-P., “IMGT Locus on Focus: A new section of Experimental and Clinical Immunogenetics”, Exp. Clin. Immunogenet., 15, 1-7 (1998); Lefranc and Lefranc, The T Cell Receptor FactsBook; and M.-P. Lefranc/Developmental and Comparative Immunology 27 (2003) 55-77, all of which are incorporated by reference in their entirety.

An “ABP fragment” comprises a portion of an intact ABP, such as the antigen-binding or variable region of an intact ABP. ABP fragments include, for example, Fv fragments, Fab fragments, F(ab′)₂ fragments, Fab′ fragments, scFv (sFv) fragments, and scFv-Fc fragments. ABP fragments include antibody fragments. Antibody fragments can include Fv fragments, Fab fragments, F(ab′)₂ fragments, Fab′ fragments, scFv (sFv) fragments, scFv-Fc fragments, and TCR fragments.

“Fv” fragments comprise a non-covalently-linked dimer of one heavy chain variable domain and one light chain variable domain.

As used, the term “antigen binding region” or “ABR” refers to a VH domain attached, directly or indirectly, to a VL domain. An ABR may exist, in an N->C direction, as a VH domain-VL domain fragment or as a VL domain-VH domain fragment. In some embodiments, the VH domain and VL domain are connected by a linker (e.g., a peptide linker). ABRs may interact intermolecularly (for example, without limitation, as in a diabody) or intramolecularly (for example, without limitation, as in a scFv). ABRs contain all of the sequence to make a binding region. However, in some embodiments, the ABRs may not be able to generate said binding region as a monomer, but may need to heterodimerize or homodimerize to generate the binding region.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length ABP.

“F(ab′)₂” fragments contain two Fab′ fragments joined, near the hinge region, by disulfide bonds. F(ab′)₂ fragments may be generated, for example, by recombinant methods or by pepsin digestion of an intact ABP. The F(ab′) fragments can be dissociated, for example, by treatment with β-mercaptoethanol.

As to form of a linker. In some embodiments, a “hinge” is a peptide linker. A hinge can include an immunoglobulin (e.g., IgG, IgM, IgE, IgA, etc.) hinge or a variant thereof. A hinge can be a linker that links an Fc (e.g., human IgG1 Fc) to an antigen-binding domain, e.g., an ABR. In some embodiments, a hinge is a flexible stretch in the heavy chains of IgG or IgA immunoglobulin classes. In some embodiments, the region referred to as hinge initiates with the “core hinge” sequence CPPC (SEQ ID NO: 732). In some cases, regions known to those skilled in the art as upper hinge sequences are described as parts of linker sequences within this application. In some embodiments “upper hinge” refers to the sequence above (attached to the N-terminus) of the core hinge sequence.

“Single-chain Fv” or “sFv” or “scFv” fragments comprise a V_{H} domain and a V_{L} domain in a single polypeptide chain. The V_{H} and V_{L} are generally linked by a peptide linker. See Plückthun A. (1994). Any suitable linker may be used. In some embodiments, the linker is a (GGGGS)ₙ (SEQ ID NO: 99). In some embodiments, n=1, 2, 3, 4, 5, or 6. See ABPs from Escherichia coli. In Rosenberg M. & Moore G. P. (Eds.), The Pharmacology of Monoclonal ABPs vol. 113 (pp. 269-315). Springer-Verlag, New York, incorporated by reference in its entirety.

“scFv-Fc” fragments comprise an scFv attached to an Fe domain. For example, an Fe domain may be attached to the C-terminal of the scFv. The Fe domain may follow the V_{H} or V_{L}, depending on the orientation of the variable domains in the scFv (i.e., V_{H}-V_{L} or V_{L}-V_{H}). Any suitable Fe domain known in the art or described herein may be used. In some cases, the Fe domain comprises an IgG4 Fe domain.

The term “single domain antibody” refers to a molecule in which one variable domain of an ABP specifically binds to an antigen without the presence of the other variable domain. Single domain ABPs, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain ABPs are also known as sdAbs or nanobodies.

The term “Fe region” or “Fc” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described in the art or elsewhere in this disclosure.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of an ABP. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD3 (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J. Biol. Chem., 2014, 289:14392-14398; each of which is incorporated by reference in its entirety. An alternative scaffold is one type of ABP.

A “multispecific ABP” is an ABP that comprises two or more different antigen-binding domains that collectively specifically bind two or more different epitopes. The two or more different epitopes may be epitopes on the same antigen (e.g., a single HLA-PEPTIDE molecule expressed by a cell) or on different antigens (e.g., different HLA-PEPTIDE molecules expressed by the same cell, a HLA-PEPTIDE molecule and a non-HLA-PEPTIDE molecule, or an HLA peptide molecule and a cell surface molecule present on a T cells or natural killer (NK) cells). In some aspects, a multi-specific ABP binds two different epitopes (i.e., a “bispecific ABP”). In some aspects, a multi-specific ABP binds three different epitopes (i.e., a “trispecific ABP”).

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

As used, the term “diabody” refers to a dimerized antigen binding region (ABR) comprising a heavy chain variable domain (VH) and a light chain variable domain (VL). Diabodies have two antigen binding sites and can be bispecific or monospecific. (See, for example, Holliger and Winter, Cancer Immunol Immunother, 1997, 45:128-130 and Proc. Natl. Acad. Sci. USA, 1993, 90:6444-6448, each of which is incorporated by reference in its entirety).

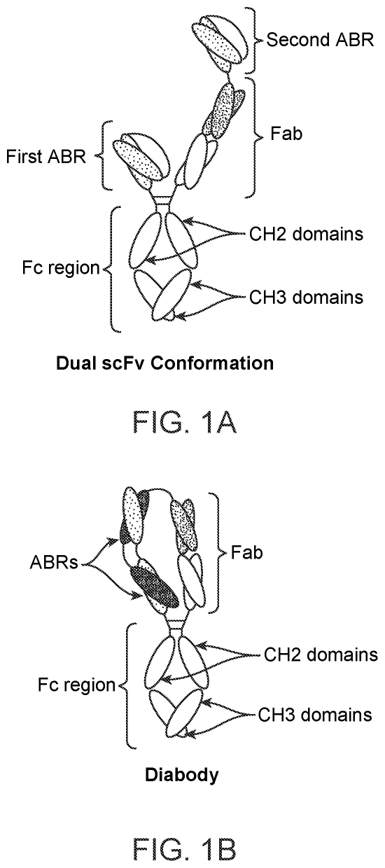

As used, the term “interacts” refers to the non-covalent pairing of VH and VL sequences either within an scFv or between a VH domain and VL domain of an ABR or set of ABRs, e.g., to form an antigen binding site. It is also contemplated that the VH domain from a first ABR can interact with the VL domain from another ABR. For example, in FIG. 1A, in each of the ABRs, a VH is shown interacting with a VL from the same polypeptide (intramolecular interaction). In the diabody of FIG. 1B, a VH from the first ABR (in the first polypeptide) is shown interacting with a VL from the second ABR (in the second polypeptide), while a VL from the first ABR (in the first polypeptide) is shown interacting with a VH from the second ABR (in the second polypeptide). These noncovalent interactions that facilitate the pairing can consist of hydrophobic, electrostatic, and van der Waals interactions. Further, in some embodiments, these noncovalent interactions may be stabilized by introduction of 2 Cys residues to form a disulfide bond (DSB) holding VH and VL together covalently. In some embodiments, this involves a Cys introduced in VH at position 44 according to Kabat numbering and a second Cys at VL100 according to Kabat numbering.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

As used herein, “homogenous” refers to a substance that is uniform throughout in composition. For example, a homogenous mixture has the same properties throughout the sample. In some embodiments, the term “homogenous” refers to a solution, suspension, or mixture (e.g. a therapeutic for treatment) having ABPs that are of the same conformation, as defined herein.

As used herein, the term “cytotoxicity” refers to the ability of antibodies, antibody fragments, and ABPs as described herein to mediate or facilitate cell death or elimination through an effector cell of the immune system (e.g., T cells and/or NK cells). For example, the term “cytotoxicity” can refer to a process by which an ABP binds an effector cell (e.g., an anti-CD3 binding domain of the ABP binds to CD3 present on an effector cell (e.g., a T cell)) and a tumor antigen binding domain of the ABP binds a target cell expressing an antigen such as a tumor antigen (e.g., a pHLA binding domain binds to a target cell expressing pHLA). Thereafter, the effector cell facilitates cell death and/or destruction (e.g., via apoptosis or lysis) of the target cell. Cytotoxic T cells, for example, can destroy the target cell through release of various molecules such as cytokines, perforin, granzymes, and proteases, which cause the target cell to undergo cell death (e.g., apoptosis). In some embodiments, the term cytotoxicity also encompasses antibody-dependent cellular cytotoxicity (also referred to as antibody-dependent cell-mediated cytotoxicity), which is an immune defense mechanism whereby effector cells of the immune system actively lyse a target cell. It is typically driven by Fc bind to Fc receptors.

As used, the term “tumor antigen” refers to refers to an antigen or portion thereof expressed only by a tumor or at a level that is higher than that expressed by normal tissue. In some embodiments, tumor antigens are exclusively expressed on tumor cells. In some embodiments, the presence or expression of a tumor antigen on normal cells is negligible. In some embodiments, these tumor antigens are expressed in a significantly higher amount on tumor cells than on normal cells. In some embodiments, the tumor antigen is an HLA-PEPTIDE.

As used, the term “target antigen” refers to an antigen or portion thereof capable of stimulating an immune response and/or being bound by a binding domain of an immune cell. Target antigens can be bound by the antigen binding site of an antibody or antibody fragment. The term target antigen encompasses, for example, cell surface molecules present on effector cells such as T cells or NK cells. In some embodiments, the target antigen is CD3. The term target antigen also encompasses tumor antigens, as described supra.

“T cells” refer to a type of lymphocyte that naturally expresses a T-cell receptor on its cell surface and plays a central role in the immune response (e.g., immune-related cell death). They differentiate into several distinct types of T cells (e.g., helper, regulatory, or cytotoxic T cells, and memory T cells). Effector T cells, for example, refer to the subset of cytotoxic T cells which are actively involved in eliminating (e.g., killing) different types of cells that are infected with pathogens, or are otherwise damaged or dysfunctional.

“Natural killer cells” or “NK cells” are a component of the innate immune system and make up approximately 15% of circulating lymphocytes. NK cells infiltrate virtually all tissues, killing target cells by means similar to cytotoxic T cells—i.e., via cytolytic granules that contain perforin and granzymes as well as via death receptor pathways. Activated NK cells also secrete inflammatory cytokines and chemokines that promote the recruitment of other leukocytes to the target tissue.

“Affinity” refers to the strength of the sum total of non-covalent interactions between a single binding site of a molecule (e.g., an ABP) and its binding partner (e.g., an antigen or epitope). Unless indicated otherwise, as used herein, “affinity” refers to intrinsic binding affinity, which reflects a 1:1 interaction between members of a binding pair (e.g., ABP and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an ABP to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the ABP to the target molecule is competitively inhibited by the control molecule. In some aspects, the affinity of a HLA-PEPTIDE ABP for a non-target molecule is less than about 50% of the affinity for HLA-PEPTIDE. In some aspects, the affinity of a HLA-PEPTIDE ABP for a non-target molecule is less than about 40% of the affinity for HLA-PEPTIDE. In some aspects, the affinity of a HLA-PEPTIDE ABP for a non-target molecule is less than about 30% of the affinity for HLA-PEPTIDE. In some aspects, the affinity of a HLA-PEPTIDE ABP for a non-target molecule is less than about 20% of the affinity for HLA-PEPTIDE. In some aspects, the affinity of a HLA-PEPTIDE ABP for a non-target molecule is less than about 10% of the affinity for HLA-PEPTIDE. In some aspects, the affinity of a HLA-PEPTIDE ABP for a non-target molecule is less than about 1% of the affinity for HLA-PEPTIDE. In some aspects, the affinity of a HLA-PEPTIDE ABP for a non-target molecule is less than about 0.1% of the affinity for HLA-PEPTIDE.

In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less).

As used in reference to antibodies, antibody fragments, and multispecific ABPs, the term “isolated” refers to one which has been separated from a component of its natural environment. In some embodiments, an antibody (for example) is purified to greater than 95% or 99% purity as determined by, for example, electrophoretic (e.g., SDS-PAGE, isoelectric focusing (IEF), capillary electrophoresis) or chromatographic (e.g., ion exchange, size exclusion, or reverse phase HPLC).

The term “k_{d}” (sec⁻¹), as used herein, refers to the dissociation rate constant of a particular ABP-antigen interaction. This value is also referred to as the k_{off} value.

The term “kₐ” (M⁻¹×sec⁻¹), as used herein, refers to the association rate constant of a particular ABP-antigen interaction. This value is also referred to as the kₒₙ value.

The term “K_{D}” (M), as used herein, refers to the dissociation equilibrium constant of a particular ABP-antigen interaction. K_{D}=k_{d}/kₐ. In some embodiments, the affinity of an ABP is described in terms of the K_{D} for an interaction between such ABP and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular ABP-antigen interaction. K_{A}=kₐ/k_{d}.

An “immunoconjugate” is an ABP conjugated to one or more heterologous molecule(s), such as a therapeutic (cytokine, for example) or diagnostic agent.

“Fc effector functions” refer to those biological activities mediated by the Fc region of an ABP having an Fc region, which activities may vary depending on isotype. Examples of ABP effector functions include C1q binding to activate complement dependent cytotoxicity (CDC), Fc receptor binding to activate ABP-dependent cellular cytotoxicity (ADCC), and ABP dependent cellular phagocytosis (ADCP).

When used herein in the context of two or more ABPs, the term “competes with” or “cross-competes with” indicates that the two or more ABPs compete for binding to an antigen (e.g., HLA-PEPTIDE). In one exemplary assay, HLA-PEPTIDE is coated on a surface and contacted with a first HLA-PEPTIDE ABP, after which a second HLA-PEPTIDE ABP is added. In another exemplary assay, a first HLA-PEPTIDE ABP is coated on a surface and contacted with HLA-PEPTIDE, and then a second HLA-PEPTIDE ABP is added. If the presence of the first HLA-PEPTIDE ABP reduces binding of the second HLA-PEPTIDE ABP, in either assay, then the ABPs compete with each other. The term “competes with” also includes combinations of ABPs where one ABP reduces binding of another ABP, but where no competition is observed when the ABPs are added in the reverse order. However, in some embodiments, the first and second ABPs inhibit binding of each other, regardless of the order in which they are added. In some embodiments, one ABP reduces binding of another ABP to its antigen by at least 25%, at least 50%, at least 60%, at least 70%, at least 80%, at least 85%, at least 90%, or at least 95%. A skilled artisan can select the concentrations of the ABPs used in the competition assays based on the affinities of the ABPs for HLA-PEPTIDE and the valency of the ABPs. The assays described in this definition are illustrative, and a skilled artisan can utilize any suitable assay to determine if ABPs compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen that specifically binds to an ABP. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP binds can be determined using known techniques for epitope determination such as, for example, testing for ABP binding to HLA-PEPTIDE variants with different point-mutations, or to chimeric HLA-PEPTIDE variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in Tables 15-17 are, in some embodiments, considered conservative substitutions for one another.

TABLE 15
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 16
Additional selected groups of amino acids that are considered conservative
substitutions for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 17
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, or example, in Creighton, Proteins: Structures and Molecular Properties 2nd ed. (1993) W. H. Freeman & Co., New York, NY. An ABP generated by making one or more conservative substitutions of amino acid residues in a parent ABP is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP provided herein. In some aspects, the disease or condition is a cancer. In some aspects, the disease or condition is a viral infection.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. In some aspects, the tumor is a solid tumor. In some aspects, the tumor is a hematologic malignancy.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

The terms “nucleic acids” and “polynucleotides” may be used interchangeably herein to refer to polymeric form of nucleotides of any length, either deoxyribonucleotides or ribonucleotides, or analogs thereof. Polynucleotides can include, but are not limited to coding or non-coding regions of a gene or gene fragment, loci (locus) defined from linkage analysis, exons, introns, messenger RNA (mRNA), cDNA, recombinant polynucleotides, branched polynucleotides, plasmids, vectors, isolated DNA, isolated RNA, nucleic acid probes, and primers. A polynucleotide may comprise modified nucleotides, such as methylated nucleotides and nucleotide analogs. Exemplary modified nucleotides include, e.g., 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthioN6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

Dual ScFv Conformation and Diabody Conformation in ABPs

The inventors of the present disclosure identified that antibodies (e.g., Format 4 antibodies) can exist in two conformations: (i) dual scFv and (ii) diabody conformation. In solution, these antibodies may exist in equilibrium between the two conformations and these two conformations may have different relative properties. For example, the two conformations in a solution may differ in terms of, but not limited to, antibody affinity to a target (e.g., to tumor-associated antigens, receptors expressed on tumor cells, receptors highly expressed on tumor cells, pHLA, etc.), cytotoxicity to diseased cells, pharmacokinetic profiles, immunogenicity, stimulation of anti-drug antibodies, etc. The inventors identified modifications, as described herein, to drive the antibodies towards a single conformation, i.e., either dual scFv conformation or diabody conformation.

The present disclosure provides ABPs comprising three polypeptides, wherein the first polypeptide comprises, in an N→C direction, a first ABR, a first hinge, a CH2 domain and a CH3 domain. The second polypeptide comprises, in an N→C direction, a VH domain of a Fab region, a CH1 domain of a Fab region, a second hinge, a CH2 domain, and a CH3 domain. The third polypeptide comprises a light chain comprising, in an N→C direction, a VL domain of the Fab region and a CL domain of the Fab region. The first and second ABRs each comprise a VH domain and a VL domain. A second ABR is attached, directly or indirectly, to the N-terminus of the second polypeptide or the third polypeptide. The first and second ABRs each comprise in an N→C direction: (i) a VH domain-a VL domain or (ii) a VL domain-VH domain. In some embodiments, the VH and VL domains of the first ABR interact with each other. In some embodiments, the VH and VL domains of the second ABR interact with each other. In some embodiments, the VH and VL domains of the first ABR interact with each other, while the VH and VL domains of the second ABR interact with each other. The hinge-CH2-CH3 domains on the first and second polypeptide constitute the Fc region of the ABP. This ABP is referred to herein as a Format 4 antibody in extended conformation, dual scFv conformation or 2×scFv conformation. (See FIG. 1A).

In some embodiments, the first ABR comprises, in an N→C direction, a VH domain of the first ABR and a VL domain of the first ABR. In some embodiments, the first ABR comprises, in an N→C direction, a VL domain of the first ABR and a VH domain of the first ABR. In some embodiments, the second ABR comprises, in an N→C direction, a VH domain of the second ABR and a VL domain of the second ABR. In some embodiments, the second ABR comprises in an N→C direction, a VL domain of the second ABR and a VH domain of the second ABR.

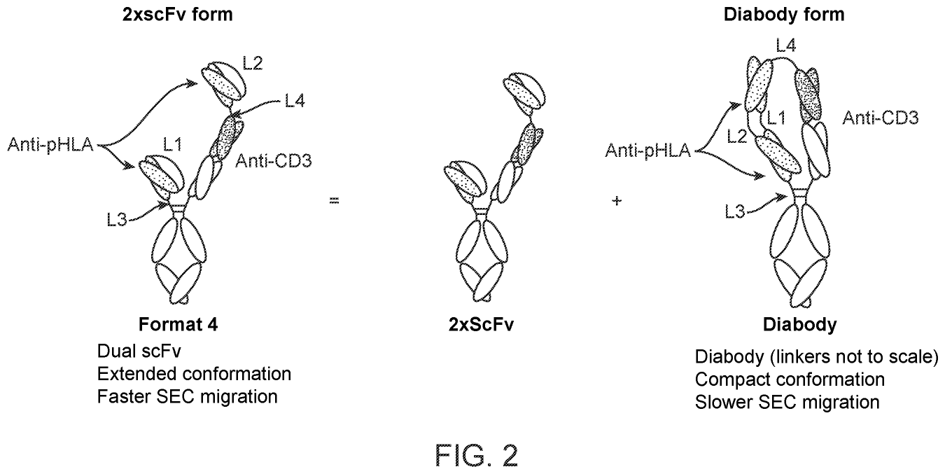

In certain embodiments, wherein the ABP is a Format 4 antibody in extended conformation, the ABPs each comprise a first ABR and a second ABR that each specifically bind an epitope of a first target antigen, a Fab that specifically binds an epitope of an additional target antigen that is distinct from the first target antigen, and an Fc domain, wherein the ABP comprises a first polypeptide, a second polypeptide, and a third polypeptide, wherein the first polypeptide comprises, in an N→C direction, a first ABR-a hinge-CH2-CH3, wherein the second polypeptide comprises, in an N→C direction, a VH domain of the Fab-a CH1 domain of the Fab-a hinge-CH2-CH3, wherein the third polypeptide comprises, in an N→C direction, a VL domain of the Fab-a CL domain of the Fab, and wherein a second ABR is attached, directly or indirectly, to the N-terminus of the second or third polypeptide. The VH domain of the first ABR is attached to the VL domain of the first ABR via a first linker (L1 of the 2×scFv form in FIG. 2) and the VH domain of the second ABR is attached to the VL domain of the second ABR via a second linker (L2 of the 2×scFv form in FIG. 2).

In some embodiments, the first target antigen is an HLA-PEPTIDE target. In some embodiments, the additional target antigen is expressed on an effector cell (e.g. T cells or NK cells). In some embodiments, the additional target antigen is a cell surface molecule expressed on an effector cells (e.g. T cells or NK cells). In some embodiments, the cell surface molecule is CD3.

The present disclosure also provides ABPs comprising three polypeptides, wherein the first polypeptide comprises, in an N→C direction, a first ABR, a first hinge, a CH2 domain, and a CH3 domain. The second polypeptide comprises, in an N→C direction, a VH domain of a Fab region, a CH1 domain of a Fab region, a second hinge, a CH2 domain, and a CH3 domain. The third polypeptide comprises a light chain comprising, in an N→C direction, a VL domain of the Fab region and a CL domain of the Fab region. A second ABR is attached, directly or indirectly, to the N-terminus of the second polypeptide or the third polypeptide. In this conformation of ABP, the VH domain of the first ABR interacts with the VL domain of the second ABR, while the VH domain of the second ABR interacts with the VL domain of the first ABR, thereby forming a diabody (see FIG. 1B). The hinge-CH2-CH3 domains on the first and second polypeptide constitute the Fc region of the ABP. This ABP is referred to herein as a Format 4 antibody in compact conformation or diabody conformation. The diabody conformation is also referred to herein as “circularized conformation” and is shown in FIG. 1B.

In some embodiments, the first ABR comprises, in an N→C direction, a VH domain of the first ABR and a VL domain of the first ABR. In some embodiments, the first ABR comprises, in an N→C direction, a VL domain of the first ABR and a VH domain of the first ABR. In some embodiments, the second ABR comprises, in an N→C direction, a VH domain of the second ABR and a VL domain of the second ABR. In some embodiments, the second ABR comprises in an N→C direction, a VL domain of the second ABR and a VH domain of the second ABR.

In certain embodiments, wherein the ABP is a Format 4 antibody in diabody conformation, the VH domain of the first ABR is attached to the VL domain of the second ABR via a first linker (e.g., L1 of the diabody form in FIG. 2) and the VH domain of the second ABR is attached to the VL domain of the first ABR via a second linker (e.g., L2 of the diabody form in FIG. 2).

In certain embodiments, wherein the ABP is a Format 4 antibody in diabody conformation, the diabody binds two epitopes of a first antigen. In some embodiments, the first target antigen is an HLA-PEPTIDE target. The VH domain and the VL domain of the Fab region bind an epitope on an additional target antigen. In some embodiments, the additional target antigen is expressed on an effector cell (e.g. T cells or NK cells). In some embodiments, the additional target antigen is a cell surface molecule expressed on an effector cells (e.g. T cells or NK cells). In some embodiments, the cell surface molecule is CD3.

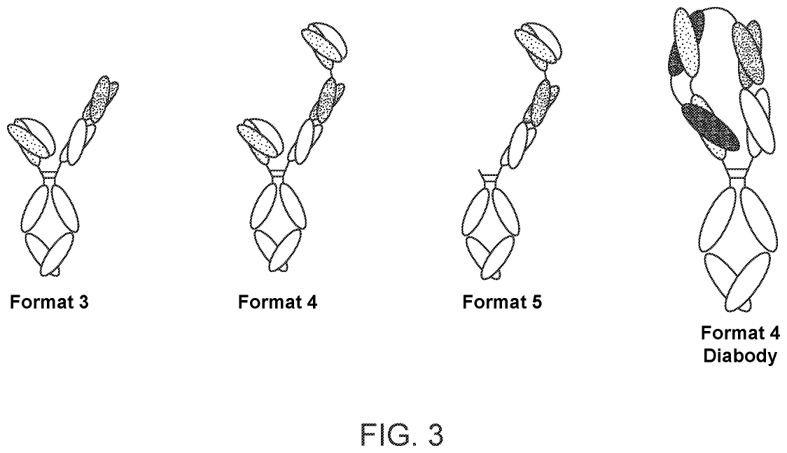

Also described herein, are Format 3 and Format 5 antibodies, as shown in FIG. 3 and described in International Application No. PCT/US2015/033076, which is incorporated by reference in its entirety.

In some embodiments, Format 4 antibodies exist in equilibrium between two conformations: (i) an extended conformation referred to as dual scFv (or 2×scFv) conformation, and (ii) compact conformation (or diabody conformation).

When a Format 4 antibody is in the dual scFv conformation (shown in FIG. 1), the VH of the first ABR interacts (pairs) with the VL of the first ABR and the VH of the second ABR interacts with the VL of the second ABR. Alternatively, when the Format 4 antibody is in compact conformation (or diabody conformation), the VH of the first ABR interacts (pairs) with the VL of the second ABR, while the VH of the second ABR interacts with the VL of the first ABR and forms a diabody.

Examples of diabodies described in the art are provided in Hollinger et al., Proc. Natl. Acad. Sci. USA, 1993, 90:6444-6448; Olafsen, T. et al. Protein Eng Des Sel., 2004, 17(1):21-27; Wu, A. et al. Protein Engineering, 2001, 14(2): 1025-1033; Asano et al., 2004, Abstract 3P-683, J. Biochem. 76(8):992; Takemura, S. et al. Protein Eng., 2000, 13(8):583-588; Baeuerle, P. A. et al. Cancer Res., 2009, 69(12):4941-4944; U.S. Pat. No. 7,129,330; and International Application No. PCT/US2015/033076, each of which is incorporated by reference in its entirety.

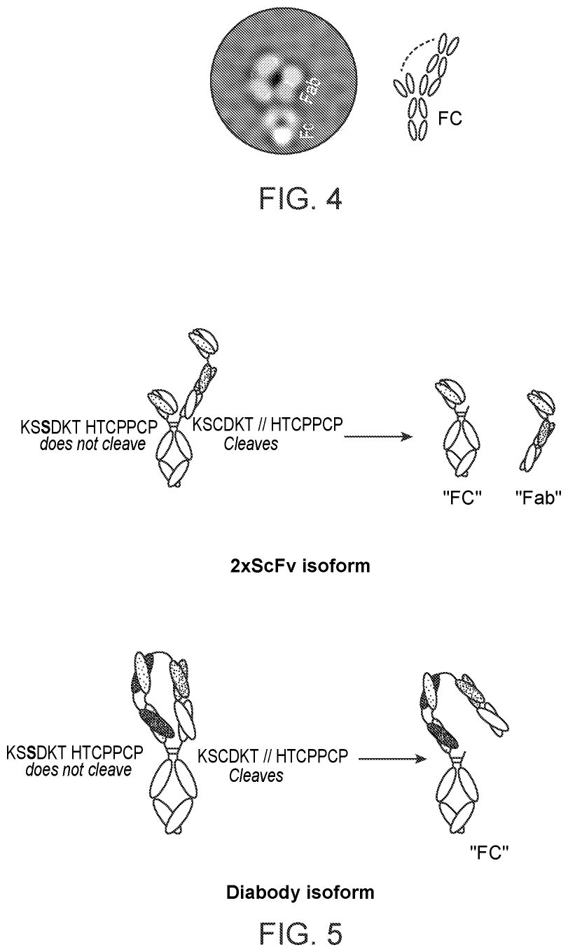

2×scFv-conformed ABPs and diabody-conformed ABPs in a solution or mixture can be visualized and differentiated using various methods known in the art, e.g., without limitation, size exclusion chromatography (SEC-HPLC or SEC) and negative stain electron microscopy (negative stain EM). For example, FIG. 4 includes a negative stain EM of a Format 4 diabody. Its compact conformation would be distinguishable from dual scFvs in a negative stain EM. When using SEC, the 2×scFv-conformed ABPs migrate faster than the diabody-conformed ABPs.

In some embodiments, when there is an equilibrium between 2×scFv-conformed ABPs and diabody-conformed ABPs, proteolysis of a purified population of the multispecific ABP with a cysteine protease that digests human IgG1 at one specific site above the hinge (e.g., KSCDKT/HTCPPC (SEQ ID NO: 2)) (e.g., proteolysis with FabALACTICA®; see FIG. 5) produces at least three species, as follows. The proteolysis produces i) a first fragment (a first species) comprising the first scFv and the Fe domain and (ii) a second fragment (a second species) comprising the second scFv and the Fab, wherein the first fragment binds to Protein A and exhibits a retention time that is greater than the retention time of the multispecific ABP which has not been digested with the cysteine protease, as measured by SEC-HPLC. The second fragment does not bind to Protein A and exhibits a retention time that is greater than the retention time of the multispecific ABP which has not been digested with the cysteine protease, as measured by SEC-HPLC. The third species produced by the proteolysis is a clipped ABP comprising the first Fv, the second Fv, and the Fab, which remains attached to the Fc domain through the diabody interaction—this is the cleaved diabody fragment (see FIG. 5, bottom right). This third species binds to Protein A and exhibits a retention time that aligns with the retention time of the multispecific ABP which has not been digested with the cysteine protease, as measured by SEC-HPLC. Exemplary methods for conducting the SEC-HPLC and the proteolytic digestion are provided in the Examples.

In some embodiments, the first target antigen is an HLA-peptide target. In some embodiments, the additional target antigen is a cell surface molecule present (e.g., expressed) on T cells or NK cells (e.g., CD3).

In certain embodiments, the ABRs that interact to form the diabody structure in the ABPs are not bispecific or multispecific. The ABRs that form a diabody in an ABP can bind the same antigen. The ABRs that form a diabody in an ABP can each have the same VH and VL domains.

In some embodiments, the ABPs described herein result in a stable (e.g. stabilized dual scFv conformation or stabilized diabody conformation) therapeutic. In some embodiments, the ABPs described herein result in a homogenous therapeutic (e.g. homogenous for stabilized dual scFv conformation or homogenous for stabilized diabody conformation).

Driving Diabody Formation with Shortened Linkers

The present disclosure provides ABPs and methods for engineering ABPs having diabody conformation by introducing “shortened linkers” at the first linker of the first ABR and second linker of the second ABR. The introduction of a shortened linker at these sites can favor diabody conformation and shift the equilibrium between 2×scFv conformation and diabody conformation towards a higher proportion of diabody conformed ABPs. In some embodiments, the introduction of a shortened linker in all of the ABPs in a mixture results in the absence of 2×scFv-conformed ABPs in that mixture of the ABPs.

As used, “shortened linker” refers to one or both of the first linker of the first ABR and second linker of the second ABR each having less than 14 amino acids. In some embodiments, “shortened linker” refers to the first linker of the first ABR and second linker of the second ABR each having 6-13 amino acids. In some embodiments, the “shortened linker” refers to a length of 3-12 amino acids. In some embodiments, the “shortened linker” refers to a length of 3-10 amino acids. In some embodiments, the “shortened linker” refers to a length of 3-8 amino acids. In some embodiments, the “shortened linker” refers to a length of 4-12 amino acids. In some embodiments, the “shortened linker” refers to a length of 4-10 amino acids. In some embodiments, the “shortened linker” refers to a length of 4-8 amino acids. In some embodiments, the “shortened linker” refers to a length of 5-12 amino acids. In some embodiments, the “shortened linker” refers to a length of 5-10 amino acids. In some embodiments, the “shortened linker” refers to a length of 5-8 amino acids. In some embodiments, the “shortened linker” refers to a length of 8-13 amino acids. In some embodiments, the “shortened linker” refers to a length of 8-12 amino acids. In some embodiments, the “shortened linker” refers to a length of 8-11 amino acids. In some embodiments, the “shortened linker” refers to a length of 8-10 amino acids. In some embodiments, the “shortened linker” refers to a length of 8-9 amino acids. In some embodiments, the “shortened linker” refers to a length selected from: 1-13; 2-13; 3-13; 4-13; 5-13; 6-13; 7-13; 8-13; 9-13; 10-13; 11-13; 12-13; 1-12; 2-12; 3-12; 4-12; 5-12; 6-12; 7-12; 8-12; 9-12; 10-12; 11-12; 1-11; 2-11; 3-11; 4-11; 5-11; 6-11; 7-11; 8-11; 9-11; 10-11; 1-10; 2-10; 3-10; 4-10; 5-10; 6-10; 7-10; 8-10; 9-10; 1-9; 2-9; 3-9; 4-9; 5-9; 6-9; 7-9; 8-9; 1-8; 2-8; 3-8; 4-8; 5-8; 6-8; 7-8; 1-7; 2-7; 3-7; 4-7; 5-7; 6-7; 1-6; 2-6; 3-6; 4-6; 5-6; 1-5; 2-5; 3-5; 4-5; 1-4; 2-4; 3-4; 1-3; 2-3; and 1-2 amino acids. In some embodiments, the “shortened linker” refers to a length of 5, 6, 7, 8, 9, or 10 amino acids. In some embodiments, the “shortened linker” refers to a length of 3, 4, 5, 6, 7, 8, 9, 10, 11, 12 or 13 amino acids. In some embodiments, the “shortened linker” refers to a length of 1, 2, 3, 4, 5, 6, 7, 8, 9, 10, 11, 12, 13, 14, 15, 16, 17, 18 or 19 amino acids. In some embodiments, the “shortened linker” refers to a length of 1 amino acid. In some embodiments, the “shortened linker” refers to a length of 2 amino acids. In some embodiments, the “shortened linker” refers to a length of 3 amino acids. In some embodiments, the “shortened linker” refers to a length of 4 amino acids. In some embodiments, the “shortened linker” refers to a length of 5 amino acids. In some embodiments, the “shortened linker” refers to a length of 6 amino acids. In some embodiments, the “shortened linker” refers to a length of 7 amino acids. In some embodiments, the “shortened linker” refers to a length of 8 amino acids. In some embodiments, the “shortened linker” refers to a length of 9 amino acids. In some embodiments, the “shortened linker” refers to a length of 10 amino acids. In some embodiments, the “shortened linker” refers to a length of 11 amino acids. In some embodiments, the “shortened linker” refers to a length of 12 amino acids. In some embodiments, the “shortened linker” refers to a length of 13 amino acids. In some embodiments, the “shortened linker” refers to a length of 14 amino acids. In some embodiments, the “shortened linker” refers to a length of 15 amino acids. In some embodiments, the “shortened linker” refers to a length of 16 amino acids. In some embodiments, the “shortened linker” refers to a length of 17 amino acids. In some embodiments, the “shortened linker” refers to a length of 18 amino acids. In some embodiments, the “shortened linker” refers to a length of 19 amino acids. It is contemplated that the first and second linker are of different lengths (number of amino acids). See Hudson, P. J., and Kortt, A. A., Journal of immunological methods 231.1-2 (1999): 177-189, which is incorporated by reference in its entirety.

In some embodiments, the first linker of the first ABR and the second linker of the second ABR are polypeptide linkers that are different lengths.

In some embodiments, in the ABPs with shortened linkers, the first ABR and second ABR bind to an HLA-peptide target with a dissociation constant (K_{D}) less than or equal to 100 nM, as measured by surface plasmon resonance (SPR; e.g., BIACORE®), biolayer interferometry (BLI; e.g., FORTEBIO®) or other methods known in the art for measuring affinity. In some embodiments, the first ABR and second ABR bind to an HLA-peptide target with a dissociation constant (K_{D}) less than or equal to about 80 nM, about 82 nM, about 84 nM, about 86 nM, about 88 nM, about 90 nM, about 92 nM, about 94 nM, about 96 nM, about 98 nM, about 100 nM, about 102 nM, about 104 nM, about 106 nM, about 108 nM, about 110 nM, about 112 nM, about 114 nM, about 116 nM, about 118 nM, about 120 nM, as measured by surface plasmon resonance (SPR; e.g., BIACORE®), biolayer interferometry (BLI; e.g., FORTEBIO®) or other methods known in the art for measuring affinity. In some embodiments, the antibody that binds to an HLA peptide target has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, in the ABPs with shortened linkers, the Fab binds to a cell surface molecule on an effector cell (e.g., a CD3 target) with a dissociation constant (K_{D}) less than or equal to 20-100 nM, as measured by surface plasmon resonance (SPR) technology (e.g., BIACORE®), biolayer interferometry (e.g., FORTEBIO®) or other methods known in the art for measuring or estimating affinity such as EC50 values derived from flow cytometry. In some embodiments, in the ABPs with shortened linkers, the Fab binds to a cell surface molecule on an effector cell (e.g., a CD3 target) with a dissociation constant (K_{D}) less than or equal to about 20 nM, about 30 nM, about 40 nM, about 50 nM, about 60 nM, about 70 nM, about 80 nM, about 90 nM, about 100 nM, about 110 nM or about 120 nM. In some embodiments, the antibody that binds to a CD3 target has a dissociation constant (K_{D}) of ≤1 M, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, the ABP binds to HLA-peptide targets (e.g. in vitro) at a higher affinity than a reference ABP. In some embodiment, the ABP binds to HLA-peptide targets (e.g. in vitro) at a the same affinity or a lower affinity than a reference ABP. In some embodiments, the ABP binds to HLA-peptide targets (e.g. in vitro) at a the same affinity or a lower affinity than a reference ABP, but has greater product homogeneity and/or stability. Stability, as used here, refers to drug stability as known in the art (e.g., stability in terms of long term storage, serum stability, through freeze-thaw cycle stability, etc.). In some embodiments, the ABP binds to a CD3 target on cells at a higher affinity than a reference ABP. In some embodiments, the ABP binds to a CD3 target on cells at a the same or a lower affinity than a reference ABP. In some embodiments, the ABP binds to a CD3 target on cells at a the same or a lower affinity than a reference ABP, but as greater product homogeneity and/or stability. Stability, as used here, refers to drug stability as known in the art (e.g., stability in terms of long term storage, serum stability, through freeze-thaw cycle stability, etc.). As used herein, the term “reference ABP” can refer to a Format 4 antibody having first and second linkers that are each greater than or equal to 14 amino acids in length. For example, the reference ABP comprises a Format 4 antibody having first and second linkers that each consist of 14, 15, 16, 17, 18, 19, 20, 21, 22, 23, 24, 25, 26, 27, 28, 29 or 30 amino acids. In some embodiments, a reference ABP refers to a Format 4 antibody having first and second linkers that each consist of (GGGGS)_{N} (SEQ ID NO: 100), wherein _{N}=3. In some embodiments, a reference ABP refers to a Format 4 antibody having first and second linkers that each consist of (GGGGS)_{N} (SEQ ID NO: 3), wherein _{N}=4. In some embodiments, the reference ABP refers to another monospecific ABP, a Format 3 antibody (see FIG. 4 and as disclosed in International application No. PCT/US2020/15736, incorporated by reference in its entirety), a Format 5 antibody (see FIG. 4 and as disclosed in International application No. PCT/US2020/15736, incorporated by reference in its entirety), a different type of bispecific or multispecific antibody.

In some embodiments, the ABP described herein has similar affinity to CD3 and/or pHLA as a reference ABP, but has greater product homogeneity and/or stability. Stability, as used here, refers to drug stability as known in the art (e.g., stability in terms of long term storage, serum stability, through freeze-thaw cycle stability, etc.).

In some embodiments, an ABP having shortened first and second linkers results in cytotoxicity once the ABP contacts a cell expressing a tumor antigen (e.g. HLA-peptide). In some embodiments, contacting the ABP with cancer cells results in at least about 10%, 20%, 30%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% cytotoxicity. In some embodiments, a concentration of 0.1 nM, 1 nM, 5 nM or 10 nM of the ABP is sufficient to result in that cytotoxicity upon contacting the ABP with the a cancer cell that expresses a tumor antigen (e.g., HLA-peptide) and an effector cell.

In some embodiments, an ABP results in greater cytotoxicity than a reference ABP, as described supra. In some embodiments, an ABP results in similar cytotoxicity to a reference ABP or less cytotoxicity than a reference ABP, as described supra. In some embodiments, the ABP described herein results in similar cytotoxicity to a reference ABP (or less cytotoxicity), but has greater product homogeneity and/or stability. Stability, as used in here, refers to drug stability as known in the art (e.g., stability in terms of long term storage, serum stability, through freeze-thaw cycle stability, etc.).

Non-limiting examples of cancer cells that express tumor antigen (e.g. HLA-peptide) include A375 cells and LN229 cells.

Linkers

Various linkers are contemplated for use in ABPs, particularly between the variable domains (variable heavy and variable light domains), between the variable regions and N-terminus of the VH domain of the Fab, and/or between the variable regions and hinge of the first polypeptide. In some embodiments, the linker is a polypeptide linker. In some embodiments, the amino acids in the polypeptide linker are selected with properties that confer flexibility and resist cleavage from proteases (e.g., glycine and serine). In some embodiments, the polypeptide linker comprises one or more glycine and/or serine residues.

In certain embodiments, the linker comprises or consists of a (GS)ₙ, (GGS)ₙ, (GGGS)ₙ (SEQ ID NO: 101), (GGSG)ₙ (SEQ ID NO: 102), (GGSGG)ₙ (SEQ ID NO: 103), and (GGGGS)ₙ sequence (SEQ ID NO: 104), wherein n is 1, 2, 3, 4, 5, 6, 7, 8, 9, 10, 11, 12, 13, 14, 15, 16, 17, 18, 19, or 20. In some embodiments, the linker comprises or consists of a (GGGGS)ₙ (SEQ ID NO: 104) sequence, wherein n is 1, 2, 3, 4, 5, 6, 7, 8, 9, 10, 11, 12, 13, 14, 15, 16, 17, 18, 19, or 20. In some embodiments. In some embodiments, n is n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20. In some embodiments, n is 1-5; 1-4, 1-3, 1-2, 2-5, 2-4, 2-3, 3-5, 3-4, or 4-5. In some embodiments, n is 1-3. In some embodiments, n is 2-3.

In some embodiments, the VH domain of the first ABR is attached to the VL domain of the second ABR via a first linker (“L1” in FIG. 2); wherein the VH domain of the second ABR is attached to the VL domain of the second ABR via a second linker (“L2” in FIG. 2) and the first and second linkers are polypeptide linkers. In some embodiments, the first and second linkers each consist of (GGGGS)_{N} (SEQ ID NO: 105), wherein _{N}=4. In some embodiments, the first and second linkers each consist of (GGGGS)_{N} (SEQ ID NO: 4), wherein _{N}=2.

In some embodiments, wherein the VH domain of the first ABR is attached to the VL domain of the second ABR via a first linker (“L1” in FIG. 2); wherein the VH domain of the second ABR is attached to the VL domain of the second ABR via a second linker (“L2” in FIG. 2), wherein the first and second linkers each consist of (GGGGS)_{N} (SEQ ID NO: 4), and wherein _{N}=2, the resulting conformation is a Format 4 antibody having a diabody conformation.

The present disclosure provides methods of producing a multispecific ABP with a diabody conformation, by creating a Format 4 antibody having shortened linkers between the VH and VL domains of the ABRs, wherein a shortened linker is a peptide linker having less than 14 amino acids (e.g., (GGGGS)_{N} (SEQ ID NO: 106), wherein _{N}=1-2).

In some embodiments, the present disclosure provides methods of producing a multispecific ABP with a diabody conformation by creating a Format 4 antibody having shortened linkers between the VH and VL domains of the ABRs, wherein a shortened linker is a peptide linker having 10 amino acids. In some embodiments, the 10 amino-acid peptide linker is (GGGGS)₂ (SEQ ID NO: 4). One of ordinary skill in the art would appreciate that (GGGGS)₂ (SEQ ID NO: 4) can also be referred to as GGGGSGGGGS (SEQ ID NO: 107) or (G₄S)₂ (SEQ ID NO: 4).

Introduction of a shortened linker, as described supra, at the first linker (L1 in FIG. 2) and second linker (L2 in FIG. 2) can shift the equilibrium of 2×scFv-conformed antibodies and diabody conformed antibody towards a higher proportion of diabody conformed antibodies. (See Examples).

Stabilizing Dual scFv Conformation with Disulfide Bonds (DSBs)

The present disclosure provides methods for stabilizing the 2×scFv conformation in the Format 4 antibodies by introducing an internal disulfide bond (internal DSB). As used herein, the term “internal DSB” refers to a DSB resulting from cysteines present in an ABP, e.g., in certain variable domains of the ABP.

In some embodiments, the ABP is a Format 4 antibody, wherein the VH domain of the first ABR is attached to the VL domain of the first ABR via a first linker (L1 in FIG. 2); wherein the VH domain of the second ABR is attached to the VL domain of the second ABR via a second linker (L2 in FIG. 2); wherein the VL domain of the first ABR is attached to the hinge in the first polypeptide via a third linker (L3 in FIG. 2); wherein the VL domain of the second ABR is attached to the N-terminus of the second polypeptide or the third polypeptide via a fourth linker (L4 in FIG. 2). In some embodiments, the first linker and the second linker each comprise 20 amino acids. In some embodiments, the first linker and the second linker each comprise 14 amino acids. For example, the first and second linker can each consist of 14, 15, 16, 17, 18, 19, 20, 21, 22, 23, 24, 25, 26, 27, 28, 29 or 30 amino acids. The first and second linker can consist of (GGGGS)_{N} (SEQ ID NO: 108), wherein _{N}=3-10. The first and second linker can consist of (GGGGS)_{N} (SEQ ID NO: 3), wherein _{N}=4. In some embodiments, the third linker and the fourth linker each consist of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids. In some embodiments, the third linker and the fourth linker each consist of 10 amino acids. For example, the third and fourth linker can consist of (GGGGS)_{X} (SEQ ID NO: 4), wherein _{X}=2. In such embodiments, the ABPs exist at an equilibrium between the extended conformation of Format 4 antibody and the diabody conformation of Format 4 antibody.

In some embodiments, when the first and second linker comprise 20 amino acids (optionally comprise 14 amino acids), the VH domain of the first ABR and/or second ABR further comprises a cysteine (Cys) at amino acid residue 44 of the VH domain according to the Kabat numbering system and wherein the VL domain of the first ABR and/or second ABR comprises a cysteine residue at amino acid residue 100 of the VL domain according to the Kabat numbering system (referred to as H44-L100 in reference to the VH (“H”) and VL(“L”) domains of the ABRs). By introducing Cys residues at both of these positions, a disulfide bond (DSB) forms that stabilizes the VH/VL interactions within each ABR. This reduces the probability that the two ABRs of the Format 4 antibodies interact to form the alternative diabody conformation. (See Examples). As a result, the 2×scFv conformation is “stabilized”. In some embodiments, this results in the absence of diabody-conformed ABPs or a negligible amount of diabody-conformed ABPs.

Other internal disulfide bonds are contemplated in the present ABPs. Non-limiting examples of other disulfide bridge mutations/positions are H105-L43, H110b-L49, H100-L50 and H101-L46. See Weatherill, E. E., et al., Protein Engineering, Design & Selection 25.7 (2012): 321-329, which is incorporated by reference in its entirety. Additional potential DSBs that are useful in the ABPs described herein can be calculated using computational modeling.

In some embodiments, in these ABPs with internal DSBs, the first ABR and second ABR bind to an HLA-peptide target with a dissociation constant (K_{D}) less than or equal to 100 nM, as measured by surface plasmon resonance (SPR) technology (e.g., BIACORE®), biolayer interferometry (e.g., FORTEBIO®) or other methods known in the art for measuring affinity. In some embodiments, the first ABR and second ABR bind to an HLA-peptide target with a dissociation constant (K_{D}) less than or equal to about 80 nM, about 82 nM, about 84 nM, about 86 nM, about 88 nM, about 90 nM, about 92 nM, about 94 nM, about 96 nM, about 98 nM, about 100 nM, about 102 nM, about 104 nM, about 106 nM, about 108 nM, about 110 nM, about 112 nM, about 114 nM, about 116 nM, about 118 nM, about 120 nM, as measured by surface plasmon resonance (SPR) technology (e.g., BIACORE®), biolayer interferometry (e.g., FORTEBIO®) or other methods known in the art for measuring affinity. In some embodiments, the antibody that binds to an HLA peptide target has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, in these ABPs with internal DSBs, the Fab binds to a cell surface molecule on an effector cell (e.g. a CD3 target) with a dissociation constant (K_{D}) less than or equal to 500 nM, preferably 20-100 nM, as measured by surface plasmon resonance (SPR) technology (e.g., BIACORE®), biolayer interferometry (e.g., FORTEBIO®) or other methods known in the art for measuring affinity. In some embodiments, the antibody that binds to CD3 target has a dissociation constant (K_{D}) of ≤1 μM, 100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, the ABP binds to HLA-peptide targets (e.g. in vitro) at a higher affinity than a reference ABP. In some embodiment, the ABP binds to HLA-peptide targets (e.g. in vitro) at a the same affinity or a lower affinity than a reference ABP. In some embodiments, the ABP binds to HLA-peptide targets (e.g. in vitro) at a the same affinity or a lower affinity than a reference ABP, but has greater product homogeneity and/or stability. Stability, as used here, refers to drug stability as known in the art (e.g., stability in terms of long term storage, serum stability, through freeze-thaw cycle stability, etc.). Stability, as used here, refers to drug stability as known in the art (e.g., stability in terms of long term storage, serum stability, through freeze-thaw cycle stability, etc.). In some embodiments, the ABP binds to a CD3 target on cells at a higher affinity than a reference ABP. In some embodiments, the ABP binds to a CD3 target on cells at a the same or a lower affinity than a reference ABP. In some embodiments, the ABP binds to a CD3 target on cells at a the same or a lower affinity than a reference ABP, but as greater product homogeneity and/or stability. Stability, as used here, refers to drug stability as known in the art (e.g., stability in terms of long term storage, serum stability, through freeze-thaw cycle stability, etc.). As used herein, the term “reference ABP” can refer to a Format 4 antibody similar to the ABP, but without the internal DSB(s). In some embodiments, the reference ABP refers to another monospecific ABP, a Format 3 antibody (as shown in FIG. 4 and as disclosed in International application No. PCT/US2020/15736, incorporated by reference in its entirety), a Format 5 antibody (as shown in FIG. 4 and as disclosed in International application No. PCT/US2020/15736, incorporated by reference in its entirety), a different type of bispecific or multispecific antibody.

In some embodiments, an ABP described herein having at least one internal DSB results in cytotoxicity once the ABP contacts a cell expressing a tumor antigen (e.g. HLA-peptide). In some embodiments, contacting the ABP with cancer cells results in at least about 10%, 20%, 30%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, cytotoxicity. In some embodiments, a concentration of 0.1 nM, 1 nM, 5 nM or 10 nM of the ABP is sufficient to result in that cytotoxicity upon contacting the ABP with a cancer cell that expresses a tumor antigen (e.g., HLA-peptide) and an effector cell.

In some embodiments, an ABP described herein results in greater cytotoxicity than a reference ABP, as described supra. In some embodiments, an ABP results in similar cytotoxicity to a reference ABP or less cytotoxicity than a reference ABP, as described supra. In some embodiments, the ABP described herein results in similar cytotoxicity to a reference ABP (or less cytotoxicity), but has greater product homogeneity and/or stability. Stability, as used here, refers to drug stability as known in the art (e.g., stability in terms of long term storage, serum stability, through freeze-thaw cycle stability, etc.).

Non-limiting examples of cancer cells that express tumor antigen (e.g. HLA-peptide) include A375 cells and LN229 cells.

Stabilizing Diabodies with Disulfide Bonds and Shortened Linkers

As described supra, the introduction of shortened linkers at the first linker of the first ABR and second linker of the second ABR of the Format 4 antibodies drives diabody formation. In addition, the introduction of an internal DSB can stabilize diabody formation. This benefit of combining the shortened linker with internal DSBs is most notable when the ABP type “breathes” and is prone to fall apart under non-reduced denaturing conditions during proteolytic digestion. As used, the term “breathing” refers to large-scale movements of secondary structures, subunits or domains or the rapid association and disassociation of antibody domains. The introduction of DSBs in the protein can reduces this breathing. (See Makowski L, et al., J Mol Biol. 2008; 375(2):529-546, which is incorporated herein by reference in its entirety).

In some embodiments, where the ABP comprises a first linker and a second linker that are each a shortened linker, as described supra, the VH domain of the first ABR and/or second ABR further comprises a cysteine (Cys) at amino acid residue 44 of the VH domain according to the Kabat numbering system and wherein the VL domain of the first ABR and/or second ABR comprises a cysteine residue at amino acid residue 100 of the VL domain according to the Kabat numbering system. The cysteine mutations that form this disulfide bond are herein referred to as “H44/L100,” “VH44/VL100,” “DSB44/100,” “H44-L100” or any other term known in the art to describe that mutation. By introducing Cys residues at both of these positions, a disulfide bond (DSB) forms that stabilizes the diabody conformation. This reduces the probability that the complexes or fragments will dissociate under non-reducing denaturing conditions during proteolytic digestion (See Examples). As a result, the diabody conformation is “stabilized”. In some embodiments, this results in the absence of 2×scFv-conformed ABPs or a negligible amount of 2×scFv-conformed ABPs.

In some embodiments, in these ABPs that combine the shortened first and second linkers with internal DSBs, the first ABR and second ABR bind to an HLA-peptide target with a dissociation constant (K_{D}) less than or equal to 100 nM, as measured by surface plasmon resonance (SPR) technology (e.g., BIACORE®), biolayer interferometry (e.g., FORTEBIO®) or other methods known in the art for measuring affinity. In some embodiments, the first ABR and second ABR bind to an HLA-peptide target with a dissociation constant (K_{D}) less than or equal to about 80 nM, about 82 nM, about 84 nM, about 86 nM, about 88 nM, about 90 nM, about 92 nM, about 94 nM, about 96 nM, about 98 nM, about 100 nM, about 102 nM, about 104 nM, about 106 nM, about 108 nM, about 110 nM, about 112 nM, about 114 nM, about 116 nM, about 118 nM, about 120 nM, as measured by surface plasmon resonance (SPR) technology (e.g., BIACORE®), biolayer interferometry (e.g., FORTEBIO®) or other methods known in the art for measuring affinity. In some embodiments, the antibody that binds to an HLA peptide target has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, in these ABPs that combine the shortened first and second linkers with internal DSBs, the Fab binds to CD3 target with a dissociation constant (K_{D}) less than or equal to 500 nM, preferably 20-100 nM, as measured by surface plasmon resonance (SPR) technology (e.g., BIACORE®), biolayer interferometry (e.g., FORTEBIO®) or other methods known in the art for measuring affinity. In some embodiments, the antibody that binds to CD3 target has a dissociation constant (K_{D}) of ≤1 μM, 100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, the ABP binds to HLA-peptide targets (e.g. in vitro) at a higher affinity than a reference ABP. In some embodiment, the ABP binds to HLA-peptide targets (e.g. in vitro) at a the same affinity or a lower affinity than a reference ABP. In some embodiments, the ABP binds to HLA-peptide targets (e.g. in vitro) at a the same affinity or a lower affinity than a reference ABP, but has greater product homogeneity and/or stability. Stability, as used here, refers to drug stability as known in the art (e.g., stability in terms of long term storage, serum stability, through freeze-thaw cycle stability, etc.). In some embodiments, the ABP binds to a CD3 target on cells at a higher affinity than a reference ABP. In some embodiments, the ABP binds to a CD3 target on cells at a the same or a lower affinity than a reference ABP. In some embodiments, the ABP binds to a CD3 target on cells at a the same or a lower affinity than a reference ABP, but as greater product homogeneity and/or stability. Stability, as used here, refers to drug stability as known in the art (e.g., stability in terms of long term storage, serum stability, through freeze-thaw cycle stability, etc.). As used herein, the term “reference ABP” can refer to a Format 4 antibody similar to the ABP, but without the internal DSB(s) and/or the shortened first and second linkers. In some embodiments, the reference ABP refers to another monospecific ABP, a Format 3 antibody (as shown in FIG. 4 and as disclosed in International application No. PCT/US2020/15736, incorporated by reference in its entirety), a Format 5 antibody (as shown in FIG. 4 and as disclosed in International application No. PCT/US2020/15736, incorporated by reference in its entirety), a different type of bispecific or multispecific antibody.

In some embodiments, the ABP described herein has similar affinity to CD3 and/or pHLA to a reference ABP, but has greater product homogeneity and/or stability. Stability, as used here, refers to drug stability as known in the art (e.g., stability in terms of long term storage, serum stability, through freeze-thaw cycle stability, etc.).

In some embodiments, an ABP described herein having shortened first and second linkers and internal DSBs results in cytotoxicity once the ABP contacts a cell expressing a tumor antigen (e.g. HLA-peptide) and an effector cell. In some embodiments, contacting the ABP with cancer cells results in at least about 10%, 20%, 30%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, cytotoxicity. In some embodiments, a concentration of 0.1 nM, 1 nM, 5 nM or 10 nM of the ABP is sufficient to result in that cytotoxicity upon contacting the ABP with the a cancer cell that expresses a tumor antigen (e.g., HLA-peptide) and an effector cell.

In some embodiments, an ABP described herein results in greater cytotoxicity than a reference ABP, as described supra. In some embodiments, an ABP results in similar cytotoxicity to a reference ABP or less cytotoxicity than a reference ABP, as described supra. In some embodiments, the ABP described herein results in similar cytotoxicity to a reference ABP (or less cytotoxicity), but has greater product homogeneity and/or stability. Stability, as used here, refers to drug stability as known in the art (e.g., stability in terms of long term storage, serum stability, through freeze-thaw cycle stability, etc.).

Non-limiting examples of cancer cells that express tumor antigen (e.g. HLA-peptide) include A375 cells and LN229 cells.

External DSBs

It is also contemplated that the ABPs described supra comprise an external DSB in place of or in addition to the internal DSBs. In some embodiments, this external DSB confers the same benefits as the internal DSBs (e.g. homogeneity, stability, affinity to target antigens, cytotoxicity, etc.). As used, the term “external DSB” refers to DSBs that result from Cys residues outside of the ABRs, for example in linkers 3 and 4, in the Fab region or in the Fc region of the ABPs. Non-limiting examples of cysteine mutations that result in external DSBs in the ABPs described herein are provided in Tables 39 and 40 (see Examples).

Exemplary sequences for ABPs having these DSB are provided in Tables 42-51. The cysteines that form the DSBs are underlined.

In some embodiments, the introduction of external DSBs stabilizes a Format 4 ABP, as described herein, in either the 2×scFv conformation or the diabody conformation. In some embodiments, the introduction of an external DSB increases the affinity of the ABP comprising that external DSB to a target antigen (e.g. pHLA, CD3) relative to a reference ABP. In some embodiments, the affinity of the ABP with the external DSB(s) is similar to that of the reference ABP, but the ABP having the external DSB(s) exhibits greater product homogeneity and/or stability. Stability, as used here, refers to drug stability as known in the art (e.g., stability in terms of long term storage, serum stability, through freeze-thaw cycle stability, etc.).

In some embodiments, the introduction of an external DSB in an ABP increases the cytotoxicity of the ABP relative to a reference ABP. For example, when the ABP having an external DSB contacts a cancer cell or a virally-infected cell, in the presence of an effector cell, the contacting results in greater cytotoxicity than a reference ABP. In some embodiments, the cytotoxicity of the ABP with the external DSB(s) is similar or lower compared to that of the reference ABP, but the ABP having the external DSB(s) exhibits greater product homogeneity and/or stability. Stability, as used here, refers to drug stability as known in the art (e.g., stability in terms of long term storage, serum stability, through freeze-thaw cycle stability, etc.).

As used herein, the term “reference ABP” refers to a Format 4 ABP lacking an external DSB or having a different number of external DSBs than the ABP claimed. In some embodiments, the reference ABP refers to another monospecific ABP, a Format 3 antibody (see FIG. 4 and as disclosed in International application No. PCT/US2020/15736, incorporated by reference in its entirety), a Format 5 antibody (see FIG. 4 and as disclosed in International application No. PCT/US2020/15736, incorporated by reference in its entirety), a different type of bispecific or multispecific antibody.

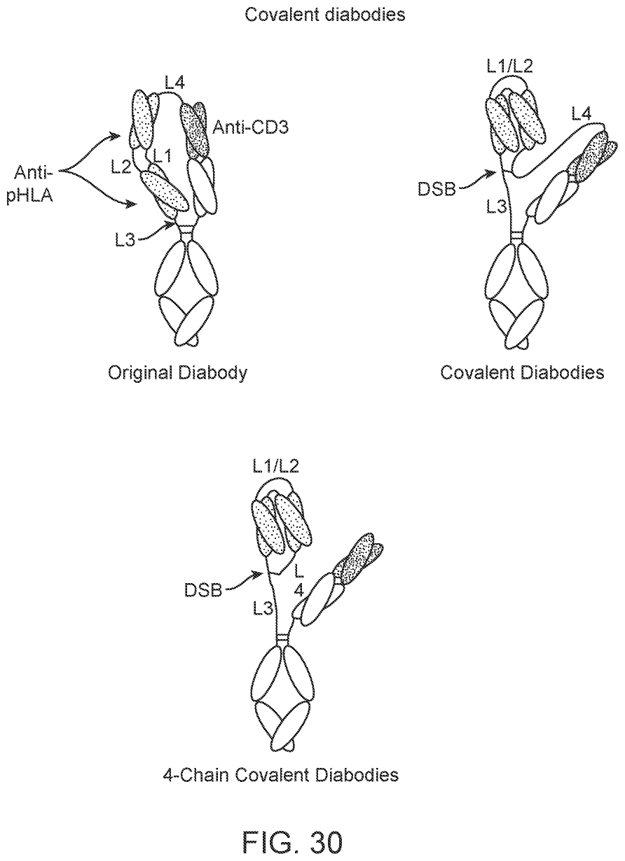

In some embodiments, the ABP having an external DSB is a covalent diabody or 4-chain covalent diabody (e.g., as shown in FIG. 30; see Examples).

Cluster of Differentiation 3 (CD3)

In some embodiments, the present disclosure provides multispecific antigen binding proteins (ABPs) comprising variable regions (e.g., in the Fab region) that bind to a cell surface molecule present on T cells, for example, cluster of differentiation 3 (CD3). CD3 is a protein complex and T cell co-receptor that is involved in activating both the cytotoxic T cell (CD8+ naive T cells) and T helper cells (CD4+ naive T cells). As used herein, the term “cluster of differentiation 3” or “CD3” refers to any native CD3 from any vertebrate source, including mammals such as primates (e.g. humans) and rodents (e.g., mice and rats), unless otherwise indicated, including, for example, CD3R, CD37, CD33a, and CD30 chains. The term encompasses “full-length,” unprocessed CD3 (e.g., unprocessed or unmodified CD3F or CD37), as well as any form of CD3 that results from processing in the cell. The term also encompasses naturally occurring variants of CD3, including, for example, splice variants or allelic variants. CD3 includes, for example, human CD3F protein (NCBI RefSeq No: NP_000724), which is 207 amino acids in length, and human CD37 protein (NCBI RefSeq No: NP_000064), which is 182 amino acids in length.

Isolated HLA-Peptide Targets

The major histocompatibility complex (MHC) is a complex of antigens encoded by a group of linked loci, which are collectively termed H-2 in the mouse and HLA in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against class I glycoproteins, while helper T-cells respond mainly against class II glycoproteins.

Human major histocompatibility complex (MHC) class I molecules, referred to interchangeably herein as HLA Class I molecules, are expressed on the surface of nearly all cells. These molecules function in presenting peptides which are mainly derived from endogenously synthesized proteins to, e.g., CD8+ T cells via an interaction with the alpha-beta T-cell receptor. The class I MHC molecule comprises a heterodimer composed of a 46-kDa α chain which is non-covalently associated with the 12-kDa light chain beta-2 microglobulin. The α chain generally comprises α1 and α2 domains which form a groove for presenting an HLA-restricted peptide, and an α3 plasma membrane-spanning domain which interacts with the CD8 co-receptor of T-cells. FIG. 1 (prior art) depicts the general structure of a Class I HLA molecule. Some TCRs can bind MHC class I independently of CD8 coreceptor (see, e.g., Kerry S E, Buslepp J, Cramer L A, et al. Interplay between TCR Affinity and Necessity of Coreceptor Ligation: High-Affinity Peptide-MHC/TCR Interaction Overcomes Lack of CD8 Engagement. Journal of immunology (Baltimore, Md: 1950). 2003; 171(9):4493-4503.)

Class I MHC-restricted peptides (also referred to interchangeably herein as HLA-restricted antigens, HLA-restricted peptides, MHC-restricted antigens, restricted peptides, or peptides) generally bind to the heavy chain alpha1-alpha2 groove via about two or three anchor residues that interact with corresponding binding pockets in the MHC molecule. The beta-2 microglobulin chain plays an important role in MHC class I intracellular transport, peptide binding, and conformational stability. For most class I molecules, the formation of a heterotrimeric complex of the MHC class I heavy chain, peptide (self, non-self, and/or antigenic) and beta-2 microglobulin leads to protein maturation and export to the cell-surface.

Binding of a given HLA subtype to an HLA-restricted peptide forms a complex with a unique and novel surface that can be specifically recognized by an ABP such as, e.g., a TCR on a T cell or an antibody or antigen-binding fragment thereof. HLA complexed with an HLA-restricted peptide is referred to herein as an HLA-PEPTIDE or HLA-PEPTIDE target. In some cases, the restricted peptide is located in the α1/α2 groove of the HLA molecule. In some cases, the restricted peptide is bound to the α1/α2 groove of the HLA molecule via about two or three anchor residues that interact with corresponding binding pockets in the HLA molecule.

Accordingly, provided herein are antigens comprising HLA-PEPTIDE targets. The HLA-PEPTIDE targets may comprise a specific HLA-restricted peptide having a defined amino acid sequence complexed with a specific HLA subtype.

HLA-PEPTIDE targets identified herein may be useful for cancer immunotherapy. In some embodiments, the HLA-PEPTIDE targets identified herein are presented on the surface of a tumor cell. The HLA-PEPTIDE targets identified herein may be expressed by tumor cells in a human subject. The HLA-PEPTIDE targets identified herein may be expressed by tumor cells in a population of human subjects. For example, the HLA-PEPTIDE targets identified herein may be shared antigens which are commonly expressed in a population of human subjects with cancer.

The HLA-PEPTIDE targets identified herein may have a prevalence with an individual tumor type The prevalence with an individual tumor type may be about 0.1%, 0.2%, 0.3%, 0.4%, 0.5%, 0.6%, 0.7%, 0.8%, 0.9%, 1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The prevalence with an individual tumor type may be about 0.1%-100%, 0.2-50%, 0.5-25%, or 1-10%.

Preferably, HLA-PEPTIDE targets are not generally expressed in most normal tissues. For example, the HLA-PEPTIDE targets may in some cases not be expressed in tissues in the Genotype-Tissue Expression (GTEx) Project, or may in some cases be expressed only in immune privileged or non-essential tissues. Exemplary immune privileged or non-essential tissues include testis, minor salivary glands, the endocervix, and the thyroid. In some cases, an HLA-PEPTIDE target may be deemed to not be expressed on essential tissues or non-immune privileged tissues if the median expression of a gene from which the restricted peptide is derived is less than 0.5 RPKM (Reads Per Kilobase of transcript per Million napped reads) across GTEx samples, if the gene is not expressed with greater than 10 RPKM across GTEX samples, if the gene was expressed at >=5 RPKM in no more two samples across all essential tissue samples, or any combination thereof.

Exemplary HLA Class I Subtypes of the HLA-PEPTIDE Targets

In humans, there are many MHC haplotypes (referred to interchangeably herein as MHC subtypes, HLA subtypes, MHC types, and HLA types). Exemplary HLA subtypes include, by way of example only, HLA-A2, HLA-A1, HLA-A3, HLA-A11, HLA-A23, HLA-A24, HLA-A25, HLA-A26, HLA-A28, HLA-A29, HLA-A30, HLA-A31, HLA-A32, HLA-A33, HLA-A34, HLA-68, HLA-B7, HLA-B8, HLA-B40, HLA-B44, HLA-B13, HLA-B15, HLA-B-18, HLA-B27, HLA-B35, HLA-B37, HLA-B38, HLA-B39, HLA-B45, HLA-B46, HLA-B49, HLA-B51, HLA-B54, HLA-B55, HLA-B56, HLA-B57, HLA-B58, HLA-C*01, HLA-C*02, HLA-C*03, HLA-C*04, HLA-C*05, HLA-C*06, HLA-C*07, HLA-C*12, HLA-C*14, HLA-C*16, HLA-Cw8, HLA-A*01:01, HLA-A*02:01, HLA-A*02:03, HLA-A*02:04, HLA-A*02:07, HLA-A*03:01, HLA-A*03:02, HLA-A*11:01, HLA-A*23:01, HLA-A*24:02, HLA-A*25:01, HLA-A*26:01, HLA-A*29:02, HLA-A*30:01, HLA-A*30:02, HLA-A*31:01, HLA-A*32:01, HLA-A*33:01, HLA-A*33:03, HLA-A*68:01, HLA-A*68:02, HLA-B*07:02, HLA-B*08:01, HLA-B*13:02, HLA-B*15:01, HLA-B*15:03, HLA-B*18:01, HLA-B*27:02, HLA-B*27:05, HLA-B*35:01, HLA-B*35:03, HLA-B*37:01, HLA-B*38:01, HLA-B*39:01, HLA-B*40:01, HLA-B*40:02, HLA-B*44:02, HLA-B*44:03, HLA-B*46:01, HLA-B*49:01, HLA-B*51:01, HLA-B*54:01, HLA-B*55:01, HLA-B*56:01, HLA-B*57:01, HLA-B*58:01, HLA-C*01:02, HLA-C*02:02, HLA-C*03:03, HLA-C*03:04, HLA-C*04:01, HLA-C*05:01, HLA-C*06:02, HLA-C*07:01, HLA-C*07:02, HLA-C*07:04, HLA-C*07:06, HLA-C*12:03, HLA-C*14:02, HLA-C*16:01, HLA-C*16:02, HLA-C*16:04, and all subtypes thereof, including, e.g., 4 digit, 6 digit, and 8 digit subtypes. As is known to those skilled in the art there are allelic variants of the above HLA types, all of which are encompassed by the present invention. A full list of HLA Class Alleles can be found on http://hla.alleles.org/alleles/. For example, a full list of HLA Class I Alleles can be found on http://hla.alleles.org/alleles/class1.html.

HLA-Restricted Peptides

The HLA-restricted peptides (referred to interchangeably herein) as “restricted peptides” can be peptide fragments of tumor-specific genes, e.g., cancer-specific genes. Preferably, the cancer-specific genes are expressed in cancer samples. Genes which are aberrantly expressed in cancer samples can be identified through a database. Exemplary databases include, by way of example only, The Cancer Genome Atlas (TCGA) Research Network: http://cancergenome.nih.gov/; the International Cancer Genome Consortium: https://dcc.icgc.org/. In some embodiments, the cancer-specific gene has an observed expression of at least 10 RPKM in at least 5 samples from the TCGA database. The cancer-specific gene may have an observable bimodal distribution.

The cancer-specific gene may have an observed expression of greater than 10, 20, 30, 40, 50, 60, 70, 80, 90, or 100 transcripts per million (TPM) in at least one TCGA tumor tissue. In certain embodiments, the cancer-specific gene has an observed expression of greater than 100 TPM in at least one TCGA tumor tissue. In some cases, the cancer specific gene has an observed bimodal distribution of expression across TCGA samples. Without wishing to be bound by theory, such bimodal expression pattern is consistent with a biological model in which there is minimal expression at baseline in all tumor samples and higher expression in a subset of tumors experiencing epigenetic dysregulation.

Preferably, the cancer-specific gene is not generally expressed in most normal tissues. For example, the cancer-specific gene may in some cases not be expressed in tissues in the Genotype-Tissue Expression (GTEx) Project, or may in some cases be expressed in immune privileged or non-essential tissues. Exemplary immune privileged or non-essential tissues include testis, minor salivary glands, the endocervix, and thyroid. In some cases, an cancer-specific gene may be deemed to not be expressed an essential tissues or non-immune privileged tissue if the median expression of the cancer-specific gene is less than 0.5 RPKM (Reads Per Kilobase of transcript per Million napped reads) across GTEx samples, if the gene is not expressed with greater than 10 RPKM across GTEX samples, if the gene was expressed at >=5 RPKM in no more two samples across all essential tissue samples, or any combination thereof.

In some embodiments, the cancer-specific gene meets the following criteria by assessment of the GTEx: (1) median GTEx expression in brain, heart, or lung is less than 0.1 transcripts per million (TPM), with no one sample exceeding 5 TPM, (2) median GTEx expression in other essential organs (excluding testis, thyroid, minor salivary gland) is less than 2 TPM with no one sample exceeding 10 TPM.

In some embodiments, the cancer-specific gene is not likely expressed in immune cells generally, e.g., is not an interferon family gene, is not an eye-related gene, not an olfactory or taste receptor gene, and is not a gene related to the circadian cycle (e.g., not a CLOCK, PERIOD, CRY gene).

The restricted peptide preferably may be presented on the surface of a tumor.

The restricted peptides may have a size of about 5, about 6, about 7, about 8, about 9, about 10, about 11, about 12, about 13, about 14, or about 15 amino molecule residues, and any range derivable therein. In particular embodiments, the restricted peptide has a size of about 8, about 9, about 10, about 11, or about 12 amino molecule residues. The restricted peptide may be about 5-15 amino acids in length, preferably may be about 7-12 amino acids in length, or more preferably may be about 8-11 amino acids in length.

Exemplary HLA-PEPTIDE Targets

Exemplary HLA-PEPTIDE targets are shown in Tables A, A1, and A2 of in International Application No. PCT/US2020/15736 or U.S. application Ser. No. 17/426,627, each of which is hereby incorporated by reference in its entirety. In each row of the Tables, the HLA allele and corresponding HLA-restricted peptide sequence of each complex is shown. The peptide sequence can consist of the respective sequence shown in any one of the rows of Tables A, A1, or A2. Alternatively, the peptide sequence can comprise the respective sequence shown in any one of the rows of Tables A, A1, or A2. Alternatively, the peptide sequence can consist essentially of the respective sequence shown in any one of the rows of Tables A, A1, or A2.

In some embodiments, the HLA-PEPTIDE target is a target as shown in Table A, A1, or A2.

In some embodiments, the HLA-PEPTIDE target is a target shown in Table A, A1, or A2, with the proviso that the isolated HLA-PEPTIDE target is not any one of Target nos. 6364-6369, 6386-6389, 6500, 6521-6524, or 6578 of Table A2, and is not an HLA-PEPTIDE target found in Table B or Table C.

In some embodiments, the HLA-restricted peptide is not from a gene selected from WT1 or MART1.

HLA Class I molecules which do not associate with a restricted peptide ligand are generally unstable. Accordingly, the association of the restricted peptide with the α1/α2 groove of the HLA molecule may stabilize the non-covalent association of the β2-microglobulin subunit of the HLA subtype with the α-subunit of the HLA subtype.

Stability of the non-covalent association of the β2-microglobulin subunit of the HLA subtype with the α-subunit of the HLA subtype can be determined using any suitable means. For example, such stability may be assessed by dissolving insoluble aggregates of HLA molecules in high concentrations of urea (e.g., about 8M urea), and determining the ability of the HLA molecule to refold in the presence of the restricted peptide during urea removal, e.g., urea removal by dialysis. Such refolding approaches are described in, e.g., Proc. Natl. Acad. Sci. USA Vol. 89, pp. 3429-3433, April 1992, hereby incorporated by reference in its entirety.

For other example, such stability may be assessed using conditional HLA Class I ligands. Conditional HLA Class I ligands are generally designed as short restricted peptides which stabilize the association of the β2 and α subunits of the HLA Class I molecule by binding to the α1/α2 groove of the HLA molecule, and which contain one or more amino acid modifications allowing cleavage of the restricted peptide upon exposure to a conditional stimulus. Upon cleavage of the conditional ligand, the β2 and α-subunits of the HLA molecule dissociate, unless such conditional ligand is exchanged for a restricted peptide which binds to the α1/α2 groove and stabilizes the HLA molecule. Conditional ligands can be designed by introducing amino acid modifications in either known HLA peptide ligands or in predicted high-affinity HLA peptide ligands. For HLA alleles for which structural information is available, water-accessibility of side chains may also be used to select positions for introduction of the amino acid modifications. Use of conditional HLA ligands may be advantageous by allowing the batch preparation of stable HLA-peptide complexes which may be used to interrogate test restricted peptides in a high throughput manner. Conditional HLA Class I ligands, and methods of production, are described in, e.g., Proc Natl Acad Sci USA. 2008 Mar. 11; 105(10): 3831-3836; Proc Natl Acad Sci USA. 2008 Mar. 11; 105(10): 3825-3830; J Exp Med. 2018 May 7; 215(5): 1493-1504; Choo, J. A. L. et al. Bioorthogonal cleavage and exchange of major histocompatibility complex ligands by employing azobenzene-containing peptides. Angew Chem Int Ed Engl 53, 13390-13394 (2014); Amore, A. et al. Development of a Hypersensitive Periodate-Cleavable Amino Acid that is Methionine- and Disulfide-Compatible and its Application in MHC Exchange Reagents for T Cell Characterisation. ChemBioChem 14, 123-131 (2012); Rodenko, B. et al. Class I Major Histocompatibility Complexes Loaded by a Periodate Trigger. J Am Chem Soc 131, 12305-12313 (2009); and Chang, C. X. L. et al. Conditional ligands for Asian HLA variants facilitate the definition of CD8+ T-cell responses in acute and chronic viral diseases. Eur J Immunol 43, 1109-1120 (2013). These references are incorporated by reference in their entirety.

Accordingly, in some embodiments, the ability of an HLA-restricted peptide described herein, e.g., described in Table A, A1, or A2, to stabilize the association of the β2- and α-subunits of the HLA molecule, is assessed by performing a conditional ligand mediated-exchange reaction and assay for HLA stability. HLA stability can be assayed using any suitable method, including, e.g., mass spectrometry analysis, immunoassays (e.g., ELISA), size exclusion chromatography, and HLA multimer staining followed by flow cytometry assessment of T cells.

Other exemplary methods for assessing stability of the non-covalent association of the 02-microglobulin subunit of the HLA subtype with the α-subunit of the HLA subtype include peptide exchange using dipeptides. Peptide exchange using dipeptides has been described in, e.g., Proc Natl Acad Sci USA. 2013 Sep. 17, 110(38):15383-8; Proc Natl Acad Sci USA. 2015 Jan. 6, 112(1):202-7, which is hereby incorporated by reference in its entirety.

Provided herein are useful antigens comprising an HLA-PEPTIDE target. The HLA-PEPTIDE targets may comprise a specific HLA-restricted peptide having a defined amino acid sequence complexed with a specific HLA subtype allele.

The HLA-PEPTIDE target may be isolated and/or in substantially pure form. For example, the HLA-PEPTIDE targets may be isolated from their natural environment, or may be produced by means of a technical process. In some cases, the HLA-PEPTIDE target is provided in a form which is substantially free of other peptides or proteins.

THE HLA-PEPTIDE targets may be presented in soluble form, and optionally may be a recombinant HLA-PEPTIDE target complex. The skilled artisan may use any suitable method for producing and purifying recombinant HLA-PEPTIDE targets. Suitable methods include, e.g., use of E. coli expression systems, insect cells, and the like. Other methods include synthetic production, e.g., using cell free systems. An exemplary suitable cell free system is described in WO2017089756, which is hereby incorporated by reference in its entirety.

Also provided herein are compositions comprising an HLA-PEPTIDE target.

In some cases, the composition comprises an HLA-PEPTIDE target attached to a solid support. Exemplary solid supports include, but are not limited to, beads, wells, membranes, tubes, columns, plates, sepharose, magnetic beads, and chips. Exemplary solid supports are described in, e.g., Catalysts 2018, 8, 92; doi:10.3390/catal8020092, which is hereby incorporated by reference in its entirety.

The HLA-PEPTIDE target may be attached to the solid support by any suitable methods known in the art. In some cases, the HLA-PEPTIDE target is covalently attached to the solid support.

In some cases, the HLA-PEPTIDE target is attached to the solid support by way of an affinity binding pair. Affinity binding pairs generally involved specific interactions between two molecules. A ligand having an affinity for its binding partner molecule can be covalently attached to the solid support, and thus used as bait for immobilizing Common affinity binding pairs include, e.g., streptavidin and biotin, avidin and biotin; polyhistidine tags with metal ions such as copper, nickel, zinc, and cobalt; and the like.

The HLA-PEPTIDE target may comprise a detectable label.

Pharmaceutical compositions comprising HLA-PEPTIDE targets.

The composition comprising an HLA-PEPTIDE target may be a pharmaceutical composition. Such a composition may comprise multiple HLA-PEPTIDE targets. Exemplary pharmaceutical compositions are described herein. The composition may be capable of eliciting an immune response. The composition may comprise an adjuvant. Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418). HLA surface expression and processing of intracellular proteins into peptides to present on HLA can also be enhanced by interferon-gamma (IFN-7). See, e.g., York I A, Goldberg A L, Mo X Y, Rock K L. Proteolysis and class I major histocompatibility complex antigen presentation. Immunol Rev. 1999; 172:49-66; and Rock K L, Goldberg A L. Degradation of cell proteins and the generation of MHC class I-presented peptides. Ann Rev Immunol. 1999; 17: 12. 739-779, which are incorporated herein by reference in their entirety.

HLA-Peptide ABPs

Also provided herein are ABPs, e.g., ABPs that specifically bind to HLA-PEPTIDE target as disclosed herein.

The HLA-PEPTIDE target may be expressed on the surface of any suitable target cell including a tumor cell.

The ABP can specifically bind to a human leukocyte antigen (HLA)-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule.

In some aspects, the ABP does not bind HLA class I in the absence of HLA-restricted peptide. In some aspects, the ABP does not bind HLA-restricted peptide in the absence of human MHC class I. In some aspects, the ABP binds tumor cells presenting human MHC class I being complexed with HLA-restricted peptide, optionally wherein the HLA restricted peptide is a tumor antigen characterizing the cancer.

An ABP can bind to each portion of an HLA-PEPTIDE complex (i.e., HLA and peptide representing each portion of the complex), which when bound together form a novel target and protein surface for interaction with and binding by the ABP, distinct from a surface presented by the peptide alone or HLA subtype alone. Generally the novel target and protein surface formed by binding of HLA to peptide does not exist in the absence of each portion of the HLA-PEPTIDE complex.

An ABP can be capable of specifically binding a complex comprising HLA and an HLA-restricted peptide (HLA-PEPTIDE), e.g., derived from a tumor. In some aspects, the ABP does not bind HLA in an absence of the HLA-restricted peptide derived from the tumor. In some aspects, the ABP does not bind the HLA-restricted peptide derived from the tumor in an absence of HLA. In some aspects, the ABP binds a complex comprising HLA and HLA-restricted peptide when naturally presented on a cell such as a tumor cell.

In some embodiments, an ABP provided herein modulates binding of HLA-PEPTIDE to one or more ligands of HLA-PEPTIDE.

The ABP may specifically bind to any one of the HLA-PEPTIDE targets as disclosed in Table A, A1, or A2. In some embodiments, the HLA-restricted peptide is not from a gene selected from WT1 or MART1. In some embodiments, the ABP does not specifically bind to any one of Target nos. 6364-6369, 6386-6389, 6500, 6521-6524, or 6578 and does not specifically bind to an HLA-PEPTIDE target found in Table B or Table C.

In more particular embodiments, the ABP specifically binds to an HLA-PEPTIDE target selected from any one of: HLA subtype A*02:01 complexed with an HLA-restricted peptide comprising the sequence LLASSILCA (SEQ ID NO: 8), HLA subtype A*01:01 complexed with an HLA-restricted peptide comprising the sequence EVDPIGHLY (SEQ ID NO: 109), HLA subtype B*44:02 complexed with an HLA-restricted peptide comprising the sequence GEMSSNSTAL (SEQ ID NO: 110), HLA subtype A*02:01 complexed with an HLA-restricted peptide comprising the sequence GVYDGEEHSV (SEQ ID NO: 111), HLA subtype *01:01 complexed with an HLA-restricted peptide comprising the sequence EVDPIGHVY (SEQ ID NO: 9), HLA subtype HLA-A*01:01 complexed with an HLA-restricted peptide comprising the sequence NTDNNLAVY (SEQ ID NO: 5), HLA subtype B*35:01 complexed with an HLA-restricted peptide comprising the sequence EVDPIGHVY (SEQ ID NO: 9), HLA subtype A*02:01 complexed with an HLA-restricted peptide comprising the sequence AIFPGAVPAA (SEQ ID NO: 6), and HLA subtype A*01:01 complexed with an HLA-restricted peptide comprising the sequence ASSLPTTMNY (SEQ ID NO: 7).

In more particular embodiments, the ABP specifically binds to an HLA-PEPTIDE target selected from any one of: HLA subtype A*02:01 complexed with an HLA-restricted peptide consisting essentially of the sequence LLASSILCA (SEQ ID NO: 8), HLA subtype A*01:01 complexed with an HLA-restricted peptide consisting essentially of the sequence EVDPIGHLY (SEQ ID NO: 109), HLA subtype B*44:02 complexed with an HLA-restricted peptide consisting essentially of the sequence GEMSSNSTAL (SEQ ID NO: 110), HLA subtype A*02:01 complexed with an HLA-restricted peptide consisting essentially of the sequence GVYDGEEHSV (SEQ ID NO: 111), HLA subtype *01:01 complexed with an HLA-restricted peptide consisting essentially of the sequence EVDPIGHVY (SEQ ID NO: 9), HLA subtype HLA-A*01:01 complexed with an HLA-restricted peptide consisting essentially of the sequence NTDNNLAVY (SEQ ID NO: 5), HLA subtype B*35:01 complexed with an HLA-restricted peptide consisting essentially of the sequence EVDPIGHVY (SEQ ID NO: 9), HLA subtype A*02:01 complexed with an HLA-restricted peptide consisting essentially of the sequence AIFPGAVPAA (SEQ ID NO: 6), and HLA subtype A*01:01 complexed with an HLA-restricted peptide consisting essentially of the sequence ASSLPTTMNY (SEQ ID NO: 7).

In more particular embodiments, the ABP specifically binds to an HLA-PEPTIDE target selected from any one of: HLA subtype A*02:01 complexed with an HLA-restricted peptide consisting of the sequence LLASSILCA (SEQ ID NO: 8), HLA subtype A*01:01 complexed with an HLA-restricted peptide consisting of the sequence EVDPIGHLY (SEQ ID NO: 109), HLA subtype B*44:02 complexed with an HLA-restricted peptide consisting of the sequence GEMSSNSTAL (SEQ ID NO: 110), HLA subtype A*02:01 complexed with an HLA-restricted peptide consisting of the sequence GVYDGEEHSV (SEQ ID NO: 111), HLA subtype *01:01 complexed with an HLA-restricted peptide consisting of the sequence EVDPIGHVY (SEQ ID NO: 9), HLA subtype HLA-A*01:01 complexed with an HLA-restricted peptide consisting of the sequence NTDNNLAVY (SEQ ID NO: 5), HLA subtype B*35:01 complexed with an HLA-restricted peptide consisting of the sequence EVDPIGHVY (SEQ ID NO: 9), HLA subtype A*02:01 complexed with an HLA-restricted peptide consisting of the sequence AIFPGAVPAA (SEQ ID NO: 6), and HLA subtype A*01:01 complexed with an HLA-restricted peptide consisting of the sequence ASSLPTTMNY (SEQ ID NO: 7).

In some embodiments, an ABP is an ABP that competes with an illustrative ABP provided herein. In some aspects, the ABP that competes with the illustrative ABP provided herein binds the same epitope as an illustrative ABP provided herein.

In some embodiments, the ABPs described herein are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining ABPs. In some embodiments, a variant is derived from any of the sequences provided herein, wherein one or more conservative amino acid substitutions are made. In some embodiments, a variant is derived from any of the sequences provided herein, wherein one or more nonconservative amino acid substitutions are made. Conservative amino acid substitutions are described herein. Exemplary nonconservative amino acid substitutions include those described in J Immunol. 2008 May 1; 180(9):6116-31, which is hereby incorporated by reference in its entirety. In certain embodiments, the non-conservative amino acid substitution does not interfere with or inhibit the biological activity of the functional variant. In yet more embodiments, the non-conservative amino acid substitution enhances the biological activity of the functional variant, such that the biological activity of the functional variant is increased as compared to the parent ABP.

ABPs Comprising an Antibody or Antigen-Binding Fragment Thereof

An ABP may comprise an antibody or antigen-binding fragment thereof.

In some embodiments, the ABPs provided herein comprise a light chain. In some aspects, the light chain is a Kappa light chain. In some aspects, the light chain is a lambda light chain.

In some embodiments, the ABPs provided herein comprise a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the ABPs provided herein comprise an antibody fragment. In some embodiments, the ABPs provided herein consist of an antibody fragment. In some embodiments, the ABPs provided herein consist essentially of an antibody fragment. In some aspects, the ABP fragment is an Fv fragment. In some aspects, the ABP fragment is a Fab fragment. In some aspects, the ABP fragment is a F(ab′)₂ fragment. In some aspects, the ABP fragment is a Fab′ fragment. In some aspects, the ABP fragment is an scFv (sFv) fragment. In some aspects, the ABP fragment is an scFv-Fc fragment. In some aspects, the ABP fragment is a fragment of a single domain ABP.

In some embodiments, an ABP fragment provided herein is derived from an illustrative ABP provided herein. In some embodiments, an ABP fragments provided herein is not derived from an illustrative ABP provided herein and may, for example, be isolated de novo according to the methods provided herein for obtaining ABP fragments.

In some embodiments, an ABP fragment provided herein retains the ability to bind the HLA-PEPTIDE target, as measured by one or more assays or biological effects described herein. In some embodiments, an ABP fragment provided herein retains the ability to prevent HLA-PEPTIDE from interacting with one or more of its ligands, as described herein.

In some embodiments, the ABPs provided herein are monoclonal ABPs. In some embodiments, the ABPs provided herein are polyclonal ABPs.

In some embodiments, the ABPs provided herein comprise a chimeric ABP. In some embodiments, the ABPs provided herein consist of a chimeric ABP. In some embodiments, the ABPs provided herein consist essentially of a chimeric ABP. In some embodiments, the ABPs provided herein comprise a humanized ABP. In some embodiments, the ABPs provided herein consist of a humanized ABP. In some embodiments, the ABPs provided herein consist essentially of a humanized ABP. In some embodiments, the ABPs provided herein comprise a human ABP. In some embodiments, the ABPs provided herein consist of a human ABP. In some embodiments, the ABPs provided herein consist essentially of a human ABP.

In some embodiments, the ABPs provided herein comprise an alternative scaffold. In some embodiments, the ABPs provided herein consist of an alternative scaffold. In some embodiments, the ABPs provided herein consist essentially of an alternative scaffold. Any suitable alternative scaffold may be used. In some aspects, the alternative scaffold is selected from an Adnectin™, an iMab, an Anticalin®, an EETI-II/AGRP, a Kunitz domain, a thioredoxin peptide aptamer, an Affibody®, a DARPin, an Affilin, a Tetranectin, a Fynomer, and an Avimer.

Also disclosed herein is an isolated humanized, human, or chimeric ABP that competes for binding to an HLA-PEPTIDE with an ABP disclosed herein.

Also disclosed herein is an isolated humanized, human, or chimeric ABP that binds an HLA-PEPTIDE epitope bound by an ABP disclosed herein.

In certain aspects, an ABP comprises a human Fc region comprising at least one modification that reduces binding to a human Fc receptor.

It is known that when an ABP is expressed in cells, the ABP is modified after translation. Examples of the posttranslational modification include cleavage of lysine at the C terminus of the heavy chain by a carboxypeptidase; modification of glutamine or glutamic acid at the N terminus of the heavy chain and the light chain to pyroglutamic acid by pyroglutamylation; glycosylation; oxidation; deamidation; and glycation, and it is known that such posttranslational modifications occur in various ABPs (See Journal of Pharmaceutical Sciences, 2008, Vol. 97, p. 2426-2447, incorporated by reference in its entirety). In some embodiments, an ABP is an ABP or antigen-binding fragment thereof which has undergone posttranslational modification. Examples of an ABP or antigen-binding fragment thereof which have undergone posttranslational modification include an ABP or antigen-binding fragments thereof which have undergone pyroglutamylation at the N terminus of the heavy chain variable region and/or deletion of lysine at the C terminus of the heavy chain. It is known in the art that such posttranslational modification due to pyroglutamylation at the N terminus and deletion of lysine at the C terminus does not have any influence on the activity of the ABP or fragment thereof (Analytical Biochemistry, 2006, Vol. 348, p. 24-39, incorporated by reference in its entirety).

### Multispecific ABPs

In some embodiments, the ABPs provided herein are multispecific ABPs.

In some embodiments, a multispecific ABP provided herein binds more than one antigen. In some embodiments, a multispecific ABP binds 2 antigens. In some embodiments, a multispecific ABP binds 3 antigens. In some embodiments, a multispecific ABP binds 4 antigens. In some embodiments, a multispecific ABP binds 5 antigens.

In some embodiments, a multispecific ABP provided herein binds more than one epitope on a HLA-PEPTIDE antigen. In some embodiments, a multispecific ABP binds 2 epitopes on a HLA-PEPTIDE antigen. In some embodiments, a multispecific ABP binds 3 epitopes on a HLA-PEPTIDE antigen.

In some embodiments, the multispecific ABP comprises an antigen-binding domain (ABD) that specifically binds to an HLA-PEPTIDE target and an additional ABD that binds to an additional target antigen. The HLA-PEPTIDE target may be a target selected from Table A, Table A1, or Table A2.

In some embodiments, the additional target antigen is a cell surface molecule present on a T cell or natural killer (NK) cell. In some embodiments, the additional target antigen is a cell surface molecule present on a T cell. In some embodiments, the additional target antigen is a cell surface molecule present on an NK cell.

In some embodiments, the cell surface molecule present on the T cell is CD3, optionally CD3ε.

The additional ABD may be an antibody or antigen-binding fragment thereof that binds to CD3, optionally CD3F. Antibodies that specifically bind CD3, e.g., CD3F include, e.g., foralumab, which is described in U.S. Pat. No. 9,850,304, which is fully incorporated by reference in its entirety. Other exemplary CD3 antibodies include OKT3. Other exemplary CD3 antibodies include humanized versions of OKT3. Other exemplary CD3 antibodies include SP34. Other exemplary CD3 antibodies include humanized versions of SP34. Other exemplary CD3 antibodies include CRIS7. OKT3 is described in Kung P et al., Monoclonal antibodies defining distinctive human T cell surface antigens. Science 206(4416), 347-349 (1979), which is hereby incorporated by reference in its entirety. Other CD3 antibodies and antigen-binding fragments are described in Kuhn and Weiner, Immunotherapy (2016) 8(8), 889-906, which is hereby incorporated by reference in its entirety.

In some embodiments, the additional ABD comprises the VH sequence

(SEQ ID NO: 112)
QVQLVESGGGVVQPGRSLRLSCAASGFTFRSYGMHWVRQA
PGKGLEWVAIIWYDGSKKNYADSVKGRFTISRDNSKNTLY
LQMNSLRAEDTAVYYCARGTGYNWFDPWGQGTLVTVSS
and
the VL sequence
(SEQ ID NO: 113)
EIVLTQSPRTLSLSPGERATLSCRASQSVSSSYLAWYQQK
PGQAPRLLIYGASSRATGIPDRFSGSGSGTDFTLTISRLD
PEDFAVYYCQQYGSSPITFGQGTRLEIK.

In some embodiments, the additional ABD comprises a VH CDR1 comprising the amino acid sequence SYGMH (SEQ ID NO: 114); a VH CDR2 comprising the amino acid sequence of IIWYDGSKKNYADSVKG (SEQ ID NO: 115); a VH CDR3 comprising the amino acid sequence of GTGYNWFDP (SEQ ID NO: 116); a VL CDR1 comprising the amino acid sequence of RASQSVSSSYLA (SEQ ID NO: 117); a VL CDR2 comprising the amino acid sequence of GASSRAT (SEQ ID NO: 118); and a VL CDR3 comprising the amino acid sequence of QQYGSSPIT (SEQ ID NO: 119), according to the Kabat or Chothia numbering scheme.

In some embodiments, the additional ABD comprises the VH sequence

(SEQ ID NO: 112)
QVQLVESGGGVVQPGRSLRLSCAASGFTFRSYGMHWVRQA
PGKGLEWVAIIWYDGSKKNYADSVKGRFTISRDNSKNTLY
LQMNSLRAEDTAVYYCARGTGYNWFDPWGQGTLVTVSS
and
the VL sequence
(SEQ ID NO: 113)
EIVLTQSPRTLSLSPGERATLSCRASQSVSSSYLAWYQQK
PGQAPRLLIYGASSRATGIPDRFSGSGSGTDFTLTISRLD
PEDFAVYYCQQYGSSPITFGQGTRLEIK.

In some embodiments, the additional ABD comprises a VH CDR1 comprising the amino acid sequence RYTMH (SEQ ID NO: 120); a VH CDR2 comprising the amino acid sequence YINPSRGYTNYNQKFKD (SEQ ID NO: 121); a VH CDR3 comprising the amino acid sequence YYDDHYSLDY (SEQ ID NO: 122); a VL CDR1 comprising the amino acid sequence SASSSVSYMN (SEQ ID NO: 123); a VL CDR2 comprising the amino acid sequence DTSKLAS (SEQ ID NO: 124); and a VL CDR3 comprising the amino acid sequence QQWSSNPFT (SEQ ID NO: 125), according to the Kabat numbering system.

In some embodiments, the additional ABD comprises the VH sequence

(SEQ ID NO: 126)
QVQLVQSGAEVKKPGASVKVSCKASGYTFTRYTMHWVRQA
PGQGLEWMGYINPSRGYTNYNQKFKDRVTLTTDKSSSTAY
MELSSLRSEDTAVYYCARYYDDHYSLDYWGQGTLVTVSS
and
the VL sequence
(SEQ ID NO: 127)
DIQMTQSPSSLSASVGDRVTITCSASSSVSYMNWYQQKPG
KAPKRLIYDTSKLASGVPSRFSGSGSGTDFTLTISSLQPE
DFATYYCQQWSSNPFTFGQGTKLEIK.

In some embodiments, the additional ABD comprises a VH CDR1 comprising the amino acid sequence YTFTRYTMH (SEQ ID NO: 128); a VH CDR2 comprising the amino acid sequence GYINPSRGYTNYN (SEQ ID NO: 129); a VH CDR3 comprising the amino acid sequence CARYYDDHYSLDYW (SEQ ID NO: 130); a VL CDR1 comprising the amino acid sequence SASSSVSYMN (SEQ ID NO: 123); a VL CDR2 comprising the amino acid sequence DTSKLAS (SEQ ID NO: 124); and a VL CDR3 comprising the amino acid sequence CQQWSSNPFTF (SEQ ID NO: 131), according to the Kabat numbering scheme.

In some embodiments, the additional ABD comprises the VH sequence

(SEQ ID NO: 132)
EVOLVESGGGLVQPGGSLRLSCAASGFTFSTYAMNWVRQA
PGKGLEWVGRIRSKYNNYATYYADSVKGRFTISRDDSKNT
LYLQMNSLRAEDTAVYYCVRHGNFGDSYVSWFAYWGQGTL
VTVSS
and
the VL sequence
(SEQ ID NO: 133)
QAVVTQEPSLTVSPGGTVTLTCGSSTGAVTTSNYANWVQQ
KPGKSPRGLIGGTNKRAPGVPARFSGSLLGGKAALTISGA
QPEDEADYYCALWYSNHWVFGGGTKLTVL.

In some embodiments, the additional ABD comprises a VH CDR1 comprising the amino acid sequence FTFSTYAMNWVRQAPGKGLE (SEQ ID NO: 134); a VH CDR2 comprising the amino acid sequence TYYADSVKGRFTISRD (SEQ ID NO: 135); a VH CDR3 comprising the amino acid sequence CVRHGNFGDSYVSWFAYW (SEQ ID NO: 136); a VL CDR1 comprising the amino acid sequence GSSTGAVTTSNYAN (SEQ ID NO: 137); a VL CDR2 comprising the amino acid sequence GTNKRAP (SEQ ID NO: 138); and a VL CDR3 comprising the amino acid sequence CALWYSNHWVF (SEQ ID NO: 139), according to the Kabat numbering scheme.

In some embodiments, the additional ABD binds to an anti-CD3 referred to as UCHT1v9. For example, in such embodiments, the additional ABD comprises the VH sequence

(SEQ ID NO: 733)
EVOLVESGGGLVQPGGSLRLSCAASGYSFTGYTMNWVRQA
PGKGLEWVALINPYKGVSTYNQKFKDRFTISVDKSKNTAY
LQMNSLRAEDTAVYYCARSGYYGDSDWYFDVWGQGTLVTV
SS
and
the VL sequence
(SEQ ID NO: 734)
DIQMTQSPSSLSASVGDRVTITCRASQDIRNYLNWYQQKP
GKAPKLLIYYTSRLESGVPSRFSGSGSGTDYTLTISSLQP
EDFATYYCQQGNTLPWTFGQGTKVEIK.

The additional ABD may be an antibody or antigen-binding fragment thereof that binds to another domain of the TCR complex, such as, e.g., CD3 delta, CD3 gamma, or major domains including TCR alpha or TCR beta, or any combination thereof. The additional ABD may be an antibody or antigen-binding fragment thereof that binds to CD3 zeta, CD4, or CD8, or any combination thereof.

In some embodiments, the cell surface molecule present on the NK cell is CD16. Accordingly, the additional ABD may comprise an antibody, antigen-binding fragment thereof, or alternative scaffold that specifically binds CD16. In some embodiments, the additional ABD comprises an antibody or antigen-binding fragment thereof as described in U.S. Pat. No. 9,035,026, which is hereby incorporated by reference in its entirety.

In some embodiments, the multispecific ABP comprises an additional ABD capable of specifically binding an immunomodulatory protein, e.g., an immune checkpoint inhibitor. Exemplary immune checkpoint inhibitors include, e.g., PD1, PDL1, CTLA-4, PDL2, B7-H3, B7-H4, BTLA, BY55, VISTA, TIM3, GAL9, LAG3, KIR, 2B4, and CGEN-15049. In some embodiments, the multispecific ABP comprises an additional ABD capable of specifically binding 41BB.

In some embodiments, the multispecific ABP comprises an additional ABD capable of specifically binding an immunomodulatory protein that enhances immune function. Exemplary immunomodulatory proteins that enhance immune function include, e.g., 41BB, CD28, GITR, OX40, CD40, CD27, and ICOS.

Many multispecific ABP constructs are known in the art, and the ABPs provided herein may be provided in the form of any suitable multispecific construct.

In some embodiments, the multispecific ABP comprises an immunoglobulin comprising at least two different heavy chain variable regions each paired with a common light chain variable region (i.e., a “common light chain ABP”). The common light chain variable region forms a distinct antigen-binding domain with each of the two different heavy chain variable regions. See Merchant et al., Nature Biotechnol., 1998, 16:677-681, incorporated by reference in its entirety.

In some embodiments, the multispecific ABP comprises an immunoglobulin comprising an ABP or fragment thereof attached to one or more of the N- or C-termini of the heavy or light chains of such immunoglobulin. See Coloma and Morrison, Nature Biotechnol., 1997, 15:159-163, incorporated by reference in its entirety. In some aspects, such ABP comprises a tetravalent bispecific ABP.

In some embodiments, the multispecific ABP comprises a hybrid immunoglobulin comprising at least two different heavy chain variable regions and at least two different light chain variable regions. See Milstein and Cuello, Nature, 1983, 305:537-540; and Staerz and Bevan, Proc. Natl. Acad. Sci. USA, 1986, 83:1453-1457; each of which is incorporated by reference in its entirety.

In some embodiments, the multispecific ABP comprises immunoglobulin chains with alterations to reduce the formation of side products that do not have multispecificity. In some aspects, the ABPs comprise one or more “knobs-into-holes” modifications as described in U.S. Pat. No. 5,731,168, incorporated by reference in its entirety.

In some embodiments, the multispecific ABP comprises immunoglobulin chains with one or more electrostatic modifications to promote the assembly of Fc hetero-multimers. See WO 2009/089004, incorporated by reference in its entirety.

In some embodiments, the multispecific ABP comprises a bispecific single chain molecule. See Traunecker et al., EMBO J., 1991, 10:3655-3659; and Gruber et al., J. Immunol., 1994, 152:5368-5374; each of which is incorporated by reference in its entirety.

In some embodiments, the multispecific ABP comprises a trispecific F(ab′)3 derivative. See Tutt et al. J. Immunol., 1991, 147:60-69, incorporated by reference in its entirety.

In some embodiments, the multispecific ABP comprises a cross-linked antibody. See U.S. Pat. No. 4,676,980; Brennan et al., Science, 1985, 229:81-83; Staerz, et al. Nature, 1985, 314:628-631; and EP 0453082; each of which is incorporated by reference in its entirety.

In some embodiments, the multispecific ABP comprises antigen-binding domains assembled by leucine zippers. See Kostelny et al., J. Immunol., 1992, 148:1547-1553, incorporated by reference in its entirety.

In some embodiments, the multispecific ABP comprises complementary protein domains. In some aspects, the complementary protein domains comprise an anchoring domain (AD) and a dimerization and docking domain (DDD). In some embodiments, the AD and DDD bind to each other and thereby enable assembly of multispecific antibody structures via the “dock and lock” (DNL) approach. Antibodies of many specificities may be assembled, including bispecific antibodies, trispecific antibodies, tetraspecific antibodies, quintspecific antibodies, and hexaspecific antibodies. Multispecific antibodies comprising complementary protein domains are described, for example, in U.S. Pat. Nos. 7,521,056; 7,550,143; 7,534,866; and 7,527,787; each of which is incorporated by reference in its entirety.

In some embodiments, the multispecific ABP comprises a dual action Fab (DAF) antibody as described in U.S. Pat. Pub. No. 2008/0069820, incorporated by reference in its entirety.

In some embodiments, the multispecific ABP comprises an antibody formed by reduction of two parental molecules followed by mixing of the two parental molecules and reoxidation to assembly a hybrid structure. See Carlring et al., PLoS One, 2011, 6:e22533, incorporated by reference in its entirety.

In some embodiments, the multispecific ABP comprises a DVD-Ig™. A DVD-Ig™ is a dual variable domain immunoglobulin that can bind to two or more antigens. DVD-Igs™ are described in U.S. Pat. No. 7,612,181, incorporated by reference in its entirety.

In some embodiments, the multispecific ABP comprises a DART™. DARTs™ are described in Moore et al., Blood, 2011, 117:454-451, incorporated by reference in its entirety.

In some embodiments, the multispecific ABP comprises a DuoBody®. DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety.

In some embodiments, the multispecific ABP comprises an antibody fragment attached to another antibody or fragment. The attachment can be covalent or non-covalent. When the attachment is covalent, it may be in the form of a fusion protein or via a chemical linker. Illustrative examples of multispecific antibodies comprising antibody fragments attached to other antibodies include tetravalent bispecific antibodies, where an scFv is fused to the C-terminus of the C_{H3} from an IgG. See Coloma and Morrison, Nature Biotechnol., 1997, 15:159-163. Other examples include antibodies in which a Fab molecule is attached to the constant region of an immunoglobulin. See Miler et al., J. Immunol., 2003, 170:4854-4861, incorporated by reference in its entirety. Any suitable fragment may be used, including any of the fragments described herein or known in the art.

In some embodiments, the multispecific ABP comprises a CovX-Body. CovX-Bodies are described, for example, in Doppalapudi et al., Proc. Natl. Acad. Sci. USA, 2010, 107:22611-22616, incorporated by reference in its entirety.

In some embodiments, the multispecific ABP comprises an Fcab antibody, where one or more antigen-binding domains are introduced into an Fc region. Fcab antibodies are described in Wozniak-Knopp et al., Protein Eng. Des. Sel., 2010, 23:289-297, incorporated by reference in its entirety.

In some embodiments, the multispecific ABP comprises a TandAb® antibody. TandAb® antibodies are described in Kipriyanov et al., J. Mol. Biol., 1999, 293:41-56 and Zhukovsky et al., Blood, 2013, 122:5116, each of which is incorporated by reference in its entirety. In some embodiments, the multispecific ABP is a TandAb® comprising, in an N→C direction, a first Fv, a second Fv, a third Fv, and a fourth Fv, wherein the first Fv is attached, indirectly or directly, to the second Fv, the second Fv is attached, indirectly or directly, to the third Fv, and the third Fv is attached, indirectly or directly, to the fourth Fv. In some embodiments, the first and fourth Fvs specifically bind a cell surface marker present on a T cell or NK cell, e.g., CD3 or CD16, and the second and third Fvs specifically bind an HLA-PEPTIDE target.

In some embodiments, the multispecific ABP comprises a tandem Fab. Tandem Fabs are described in WO 2015/103072, incorporated by reference in its entirety.

In some embodiments, the multispecific ABP comprises a Zybody™. Zybodies™ are described in LaFleur et al., mAbs, 2013, 5:208-218, incorporated by reference in its entirety.

In some embodiments, the multispecific ABP is a BEAT® molecule, which is described in U.S. Pat. No. 9,683,052, and in Moretti P et al., BMC Proceedings 2013 7 (Suppl 6):O9, available at https://doi.org/10.1186/1753-6561-7-S6-O9, each of which is hereby incorporated by reference in its entirety.

In some embodiments, the multispecific ABP is a trivalent, bispecific ABP comprising a first and a second scFv that specifically binds an HLA-PEPTIDE target and a Fab fragment that specifically binds another target, e.g., a cell surface molecule present on the surface of a T cell or NK cell. In some embodiments, the multispecific ABP comprises a first polypeptide and a second polypeptide, wherein the first polypeptide comprises the first scFv and the second polypeptide comprises the second scFv and the Fab fragment, wherein the second scFv is attached, directly or indirectly, to the N-terminus of the Fab fragment. In some embodiments, the first scFv and the Fab fragment are connected, directly or indirectly, to an Fc domain, the Fc domain optionally comprising a knob-hole or other orthogonal mutation.

Also provided herein is a trivalent, bispecific ABP comprising a first and second scFv that specifically binds a first target antigen and a Fab fragment that specifically binds a second target antigen, wherein the multispecific ABP comprises a first polypeptide and a second polypeptide, wherein the first polypeptide comprises the first scFv and the second polypeptide comprises the second scFv and the Fab fragment, wherein the second scFv is attached, directly or indirectly, to the N-terminus of the Fab fragment. In some embodiments, the first scFv and the Fab fragment are connected, directly or indirectly, to an Fc domain, the Fc domain optionally comprising a knob-hole or other orthogonal mutation.

In some embodiments of the trivalent, bispecific ABP, a variable domain of the first scFv interacts with a variable domain of the second scFv. In some embodiments, the VH domain of the first scFv interacts with the VL domain of the second scFv. In some embodiments, the VL domain of the first scFv interacts with the VH domain of the second scFv. In some embodiments, the VL domain of the first scFv interacts with the VH domain of the second scFv and wherein the VH domain of the first scFv interacts with the VL domain of the second scFv. In some embodiments, the interaction of the VL domain of the first scFv with the VH domain of the second scFv and the interaction of the VH domain of the first scFv with the VL domain of the second scFv results in a circularized conformation. In some embodiments, proteolysis of a purified population of the isolated multispecific ABP with a cysteine protease that digests human IgG1 at one specific site above the hinge (KSCDKT/HTCPPC (SEQ ID NO: 2)) produces a fragment comprising the first scFv, the second scFv, and the Fab. some embodiments, the fragment comprising the first scFv, the second scFv, and the Fab binds to Protein A and exhibits a retention time that aligns with retention time of the isolated multispecific ABP which has not been digested with the cysteine protease, as measured by SEC-HPLC.

In some embodiments of the trivalent, bispecific ABP, the VL domain of the first scFv interacts with the VH domain of the first scFv, and wherein the VL domain of the second scFv interacts with the VH domain of the second scFv. In some embodiments, proteolysis of a purified population of the isolated multispecific ABP with a cysteine protease that digests human IgG1 at one specific site above the hinge (KSCDKT/HTCPPC (SEQ ID NO: 2)) produces (i) a first fragment comprising the first scFv and the Fc domain, and (ii) a second fragment comprising the second scFv and the Fab. In some embodiments, the first fragment binds to Protein A and exhibits a retention time that is greater than retention time of the isolated multispecific ABP which has not been digested with the cysteine protease, as measured by SEC-HPLC. In some embodiments, the second fragment does not bind to Protein A and exhibits a retention time that is greater than retention time of the isolated multispecific ABP which has not been digested with the cysteine protease, as measured by SEC-HPLC. In some embodiments, the VH domain of the first scFv comprises a cysteine at amino acid residue 44 of the VH domain according to the Kabat numbering system and wherein the VL domain of the first scFv comprises a cysteine residue at amino acid residue 100 of the VL domain according to the Kabat numbering system. In some embodiments, the VH domain of the second scFv comprises a cysteine at amino acid residue 44 of the VH domain according to the Kabat numbering system and wherein the VL domain of the second scFv comprises a cysteine residue at amino acid residue 100 of the VL domain according to the Kabat numbering system. In some embodiments, the VH domains of the first and second scFv each comprise a cysteine at amino acid residue 44 of the VH domain according to the Kabat numbering system and wherein the VL domain of the first and second scFv each comprise a cysteine residue at amino acid residue 100 of the VL domain according to the Kabat numbering system.

In some embodiments, the multispecific ABP comprises a first scFv and a second scFv that each specifically bind a first target antigen, a Fab that specifically binds an additional target antigen that is distinct from the first target antigen, and an Fc domain, wherein the ABP comprises a first polypeptide, a second polypeptide, and a third polypeptide, wherein the first polypeptide comprises, in an N→C direction, the first scFv-optional linker-CH2-CH3, wherein the second polypeptide comprises, in an N→C direction, a VH domain of the Fab-a CH1 domain of the Fab-CH2-CH3, wherein the third polypeptide comprises, in an N→C direction, a VL domain of the Fab-a CL domain of the Fab, and wherein the second scFv is attached, directly or indirectly, to the N-terminus of the second polypeptide or the third polypeptide, wherein the VL domain of the first scFv interacts with the VH domain of the second scFv, and wherein the VH domain of the first scFv interacts with the VL domain of the second scFv.

In some embodiments, the multispecific ABP comprises a single domain antibody. Single domain antibodies are described herein. For example, the first ABD, second ABD, or first and second ABD may comprise a single domain antibody. In some embodiments, the multispecific ABP comprises a first ABD comprising an scFv and a second ABD comprising a single domain antibody. In some embodiments, the multispecific ABP comprises a first ABD comprising a Fab and a second ABD comprising a single domain antibody. In some embodiments, the first ABD and second ABD are attached to an Fc region. In some embodiments, the multispecific ABP further comprises a third ABD which is an scFv or Fab attached, directly or indirectly, to the N-terminus of the single domain antibody. In some embodiments, the C-terminus of the first and second ABDs are attached to the N-terminus of the Fc region. In particular embodiments, the Fc region comprises one or more modifications that promote heterodimerization, e.g., a knob-in-hole modification, a charged pair mutation. In some embodiments, the single domain antibody of the first ABD is a fully human VH single domain. In some embodiments, the second ABD is capable of selectively binding a cell surface protein of a T cell, e.g., CD3, or a cell surface protein of an NK cell, e.g., CD16.

In some embodiments, the multispecific ABP comprises a human heavy chain antibody. Human heavy chain antibodies are described in Clark et al., Front Immunol. 2019 Jan. 7; 9:3037. doi: 10.3389/fimmu.2018.03037, which is incorporated by reference in its entirety.

In some embodiments, the multispecific ABP comprises an alternative scaffold. Alternative scaffolds are described herein.

In some embodiments, the multispecific ABP comprises one or more anticalins. Anticalins, as well as methods of making anticalins, are described in, e.g., U.S. Pat. Nos. 7,250,297 and 7,585,940, each of which is hereby incorporated by reference in its entirety. In some embodiments, the multispecific ABP is a multispecific anticalin-based fusion protein. Multispecific anticalin-based fusion proteins can include, e.g., multispecific Fc-anticalin proteins, pure anticalin proteins comprising two or more anticalins attached by one or more linkers, and multispecific fusion proteins comprising one or more anticalins fused, directly or indirectly, with an antibody or antigen-binding fragment thereof. Exemplary multispecific ABPs comprising one or more anticalins are described in e.g., Rothe C, Skerra A. Anticalin® Proteins as Therapeutic Agents in Human Diseases. BioDrugs. 2018; 32(3):233-243, which is hereby incorporated by reference in its entirety. In some embodiments, an anticalin of the multispecific ABP is capable of specifically binding an HLA-PEPTIDE target. In some embodiments, an anticalin of the multispecific ABP is capable of binding the additional target antigen.

In some embodiments, the multispecific ABP is a BiTE, wherein the first ABD is a first scFv and wherein the additional ABD is a second scFv. In some embodiments, the first scFv and the second scFv are attached via a linker. In some embodiments, the BiTE comprises, in an N→C direction, the first scFv-the linker-the second scFv. In some embodiments, the BiTE comprises, in an N→C direction, the second scFv-the linker-the first scFv. In some embodiments, the linker comprises (GGGGS)_{N} (SEQ ID NO: 140), wherein N=1-10. In some embodiments, N=1-4. In some embodiments, N=1.

Also provided herein is a trivalent, multispecific ABP comprising a first scFv and a second scFv that each specifically bind a first target antigen, a Fab that specifically binds a second target antigen that is distinct from the first target antigen, and an Fc domain. In some embodiments, the multispecific ABP is a trivalent, multispecific ABP comprising a first scFv and a second scFv that each specifically bind the first target antigen and a Fab that specifically binds the additional target antigen. In some embodiments, the ABP comprises a first polypeptide, a second polypeptide, and a third polypeptide, wherein the first polypeptide comprises, in an N→C direction, the first scFv-optional linker-CH2-CH3, wherein the second polypeptide comprises, in an N→C direction, a VH domain of the Fab-a CH1 domain of the Fab-CH2-CH3, wherein the third polypeptide comprises, in an N→C direction, a VL domain of the Fab-a CL domain of the Fab, and wherein the second scFv is attached, directly or indirectly, to the N-terminus of the second polypeptide or the third polypeptide. In some embodiments, the second scFv is attached, directly or indirectly, to the N-terminus of the second polypeptide. In some embodiments, the second scFv is attached, directly or indirectly, to the N-terminus of the third polypeptide. In some embodiments, the first scFv and the second scFv each bind to an HLA-PEPTIDE target. In some embodiments, the first scFv and the second scFv each bind to the same HLA-PEPTIDE target. In some embodiments, the first scFv and the second scFv each bind to the same epitope of the HLA-PEPTIDE target. In some embodiments, the first scFv and the second scFv each comprise identical CDR sequences. In some embodiments, the first scFv and the second scFv each comprise identical VH and VL sequences. In some embodiments, the linker comprises (GGGGS)_{N} (SEQ ID NO: 140), wherein _{N}=1-10. In some embodiments, _{N}=1-4. In some embodiments, _{N}=2.

In some embodiments, the multispecific ABP comprises an scFv and a Fab, wherein the ABP comprises a first polypeptide, a second polypeptide, and a third polypeptide, wherein the first polypeptide comprises, in an N→C direction, the first scFv-CH2-CH3, wherein the second polypeptide comprises, in an N→C direction, a VH domain of the Fab-a CH1 domain of the Fab-CH2-CH3, wherein the third polypeptide comprises, in an N→C direction, a VL domain of the Fab-a CL domain of the Fab. In some embodiments, the first ABD comprises the scFv and the additional ABD comprises the Fab. In some embodiments, the first ABD comprises the Fab and the additional ABD comprises the scFv. In some embodiments, the scFv is attached to CH2 via the linker. In some embodiments, the linker comprises (GGGGS)_{N} (SEQ ID NO: 140), wherein _{N}=1-10. In some embodiments, _{N}=1-4. In some embodiments, _{N}=1.

In some embodiments, the multispecific ABP comprises a first and second scFv and a first and second Fab, wherein the multispecific ABP comprises a first polypeptide, a second polypeptide, a third polypeptide, and a fourth polypeptide, wherein the first polypeptide comprises, in an N→C direction, a VH domain of the first Fab-CH1-CH2-CH3-optional linker-the first scFv, wherein the second polypeptide comprises, in an N→C direction, a VH domain of the second Fab-CH1-CH2-CH3-optional linker-the second scFv, wherein the third polypeptide comprises, in an N→C direction, a VL domain of the first Fab-a Cl domain of the first Fab, and wherein the fourth polypeptide comprises, in an N→C direction, a VL domain of the second Fab-a Cl domain of the second Fab. In some embodiments, the first scFv and the second scFv each bind to an HLA-PEPTIDE target. In some embodiments, the first scFv and the second scFv each bind to the same HLA-PEPTIDE target. In some embodiments, the first scFv and the second scFv each bind to the same epitope of the HLA-PEPTIDE target. In some embodiments, the first scFv and the second scFv each comprise identical CDR sequences. In some embodiments, the first scFv and the second scFv each comprise identical VH and VL sequences. In some embodiments, the first Fab and the second Fab each bind the additional target antigen. In some embodiments, the first Fab and the second Fab each bind to the same epitope of the additional target antigen. In some embodiments, the first Fab and the second Fab each comprise identical CDR sequences. In some embodiments, the first Fab and the second Fab each comprise identical VH and VL sequences. In some embodiments, the first and second polypeptide chains are identical and the third and fourth polypeptide chains are identical. In some embodiments, the first polypeptide comprises, in an N→C direction, a VH domain of the first Fab-CH1-CH2-CH3-linker-the first scFv. In some embodiments, the second polypeptide comprises, in an N→C direction, a VH domain of the second Fab-CH1-CH2-CH3-linker-the second scFv. In some embodiments, the linker comprises (GGGGS)_{N} (SEQ ID NO: 140), wherein _{N}=1-10. In some embodiments, _{N}=1-4. In some embodiments, _{N}=2.

In some embodiments, the multispecific ABP comprises an scFv and a Fab, wherein the ABP comprises a first polypeptide, a second polypeptide, and a third polypeptide, wherein the first polypeptide comprises, in an N→C direction, optional hinge-CH2-CH3, wherein the second polypeptide comprises, in an N→C direction, a VH domain of the Fab-a CH1 domain of the Fab-CH2-CH3, wherein the third polypeptide comprises, in an N→C direction, a VL domain of the Fab-a CL domain of the Fab, and wherein the scFv is attached, directly or indirectly, to the N-terminus of the second polypeptide or the third polypeptide. In some embodiments, the scFv is attached, directly or indirectly, to the N-terminus of the second polypeptide. In some embodiments, the scFv is attached, directly or indirectly, to the N-terminus of the third polypeptide. In some embodiments, the first ABD comprises the scFv and the additional ABD comprises the Fab. In some embodiments, the first ABD comprises the Fab and the additional ABD comprises the scFv. In some embodiments, the scFv is attached to the N-terminus of the second polypeptide or the third polypeptide via a linker. In some embodiments, the linker comprises (GGGGS)_{N} (SEQ ID NO: 140), wherein _{N}=1-10. In some embodiments, _{N}=1-4. In some embodiments, _{N}=2.

In some embodiments, the multispecific ABP comprises a first and second scFv and a first and second Fab, wherein the multispecific ABP comprises a first polypeptide, a second polypeptide, a third polypeptide, and a fourth polypeptide, wherein the first polypeptide comprises, in an N→C direction, a VH domain of the first Fab-CH1-CH2-CH3, wherein the second polypeptide comprises, in an N→C direction, a VH domain of the second Fab-CH1-CH2-CH3, wherein the third polypeptide comprises, in an N→C direction, a VL domain of the first Fab-a Cl domain of the first Fab, and wherein the fourth polypeptide comprises, in an N→C direction, a VL domain of the second Fab-a Cl domain of the second Fab, and wherein the first scFv is attached, directly or indirectly, to the N-terminus of the first or third polypeptide, and wherein the second scFv is attached, directly or indirectly, to the N-terminus of the second or fourth polypeptide. In some embodiments, the first scFv is attached, directly or indirectly, to the N-terminus of the first polypeptide. In some embodiments, the first scFv is attached, directly or indirectly, to the N-terminus of the third polypeptide. In some embodiments, the second scFv is attached, directly or indirectly, to the N-terminus of the second polypeptide. In some embodiments, the first scFv is attached, directly or indirectly, to the N-terminus of the fourth polypeptide. In some embodiments, the first scFv and the second scFv each bind to an HLA-PEPTIDE target. In some embodiments, the first scFv and the second scFv each bind to the same HLA-PEPTIDE target. In some embodiments, the first scFv and the second scFv each bind to the same epitope of the HLA-PEPTIDE target. In some embodiments, the first scFv and the second scFv each comprise identical CDR sequences. In some embodiments, the first scFv and the second scFv each comprise identical VH and VL sequences. In some embodiments, the first Fab and the second Fab each bind the additional target antigen. In some embodiments, the first Fab and the second Fab each bind to the same epitope of the additional target antigen. In some embodiments, the first Fab and the second Fab each comprise identical CDR sequences. In some embodiments, the first Fab and the second Fab each comprise identical VH and VL sequences. In some embodiments, the first and second polypeptide chains are identical and the third and fourth polypeptide chains are identical. In some embodiments, the first scFv is attached to the N-terminus of the first or third polypeptide via a linker. In some embodiments, the second scFv is attached to the N-terminus of the second or fourth polypeptide via a linker. In some embodiments, the linker comprises (GGGGS)_{N} (SEQ ID NO: 140), wherein _{N}1-10. In some embodiments, _{N}=1-4. In some embodiments, _{N}=2.

### Fc Region and Variants

An Fc region (also referred to herein as an Fc domain) can be an integral part of an antibody or Fc-fusion molecule, and can play a role in mediating effector functions such as, e.g., antibody-dependent cell-mediated cytotoxicity (ADCC), complement dependent cytotoxicity (CDC), opsonization and transcytosis.

In certain embodiments, a multispecific ABP provided herein comprises an Fc region. An Fc region can be wild-type or a variant thereof. A “wild-type Fc” refers to one comprising an amino acid sequence identical to the amino acid sequence of an Fc region found in nature. For example, wild-type human Fc regions include a wild-type-sequence human IgG1 Fc region (non-A and A allotypes); wild-type-sequence human IgG2 Fc region; wild-type sequence human IgG3 Fc region; and wild-type-sequence human IgG4 Fc region, as well as naturally occurring variants thereof. In certain embodiments, an ABP provided herein comprises an Fc region with one or more amino acid substitutions, insertions, or deletions in comparison to a naturally occurring Fc region. In some aspects, such substitutions, insertions, or deletions yield ABP with altered stability, glycosylation, or other characteristics. In some aspects, such substitutions, insertions, or deletions yield a glycosylated ABP.

A “variant Fc region,” “engineered Fc region” or “variant CH2-CH3 domain” comprises an amino acid sequence that differs from that of a native-sequence Fc region by virtue of at least one, relative amino acid modification, e.g., one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising ABP” refers to an ABP that comprises an Fc region. The C-terminal lysine (residue 447 according to the EU numbering system) of the Fc region may be removed, for example, during purification of the ABP or by recombinant engineering the nucleic acid encoding the ABP. Accordingly, an ABP having an Fc region can comprise an ABP with or without K447.

In some aspects, the Fc region of an ABP provided herein is modified to yield an ABP with altered affinity for an Fc receptor, or an ABP that is more immunologically inert. In some embodiments, the ABP variants provided herein possess some, but not all, effector functions. Such ABPs may be useful, for example, when the half-life of the ABP is important in vivo, but when certain effector functions (e.g., complement activation and ADCC) are unnecessary or deleterious.

In some embodiments, an ABP provided herein has one or more mutations to reduce an effector function. For example, an ABP may have mutations in the Fc of human IgG1 that result in reduced, substantial loss or complete loss of the ABP binding to CD64, CD32A, CD16 and C1q (FcγRI, FcγRII, FcγRIII and C1q) relative to an unmodified version of the Fc. In some embodiments, an ABP provided herein comprises a variant CH2-CH3 domain comprising one or more amino acid substitutions that reduce binding to an Fc receptor on the cell surface of an effector cell, e.g., FcγRI; FcγRIIA; FcγRIIB1; FcγRIIIB2; FcγRIIIA; FcγRIIIB receptors. In some embodiments, the reduced effector functions can include one or more of reduced complement-dependent cytotoxicity (CDC), reduced antibody-dependent cellular cytotoxicity (ADCC), and reduced complement fixation. These modifications to the Fc can prevent multispecific ABPs from causing target cell death (e.g., T cell death) or, e.g., unwanted cytokine secretion. The modification(s) can also help reduce inter-individual variation in patient response to an ABP provided herein. Disabling productive Fc receptor engagement by reducing binding to one or more Fc receptors other than FcRn, where the Fc receptor binds monomeric IgG and/or multimeric immune complexes, can restore activity to the antibody and provide an improved therapeutic profile.

Examples of Fc effector functions that can be reduced through modification include, without limitation: ability to activate classical complement; ADCC; opsonization; ability to bind FcγRI (CD64) at, e.g., a high affinity of 1×10⁻⁹M; ability to bind FcγRIIIa,b (CD16), e.g., at an affinity 5×10⁻⁵ M or higher; and ability to bind FcγRIIa,b (CD32), e.g., at an affinity of 2×10⁻⁶ M or higher. Properties of antibodies having reduced effector function via Fc silencing are described, for example, in An et al. mAbs vol. 1,6 (2009): 572-9 and Wang, et al. Protein & cell 9.1 (2018): 63-73, the relevant disclosures of each of which are herein incorporated by reference.

In some embodiments, the ABP comprises a variant CH2-CH3 domain comprising one or more amino acid substitutions which reduce Fc effector functions. In some embodiments, the one or more amino acid substitutions are in the CH2 domain at one or more of EU index positions: 234, 235, and/or 331. In some embodiments, the one or more amino acid substitutions are in the CH2 domain at EU index positions: 234, 235, and 331. In some embodiments, the one or more amino acid substitutions are selected from: L234F, L235E, and P331S, according to the EU numbering system. In some embodiments, the variant CH2-CH3 domain comprises the amino acid substitutions of L234F, L235E, and P331S (dubbed “TM” modifications or mutations), according to the EU numbering system.

Binding of IgG to the FcγRs or C1q depends on residues located in the hinge region and the CH2 domain. Substitutions in human IgG1 or IgG2 residues at positions 233-236 and IgG4 residues at positions 327, 330 and 331 have been shown to greatly reduce ADCC and CDC. The triple amino acid substitution L234A, L235A, and G237A largely eliminates FcγR and complement effector functions (see, for example, U.S. Pat. No. 9,644,025, the relevant disclosures of which are herein incorporated by reference). The LALA variant, L234A/L235A, also has significantly reduced FcγR binding; as does E233P/L234V/L235A/G236+A327G/A330S/P331S. See, for example, Armour et al. (1999) Eur J Immunol. 29(8):2613-24. The set of mutations: K322A, L234A and L235A are sufficient to almost completely abolish FcγR and C1q binding.

Additional modifications to silence the Fc region or reduce effector function may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604).

Additional mutations that reduce binding to FcγR include, without limitation, modification of the glycosylation on asparagine 297 of the Fc domain, which is known to be required for optimal FcR interaction. For example known amino acid substitutions include N297 mutations, for example N297A/Q/D/H/G/C, which changes result in the loss of a glycosylation site on the protein. Enzymatically deglycosylated Fc domains, recombinantly expressed antibodies in the presence of a glycosylation inhibitor and the expression of Fc domains in bacteria have a similar loss of glycosylation and consequent binding to FcγRs.

Additional examples of Fc silencing are known to those of ordinary skill in the art and are provided, for example, in U.S. Pat. No. 10,611,842, the relevant disclosures of which are herein incorporated by reference.

As used herein, a “silenced Fc” refers to one that has been mutagenized to retain activity with respect to, for example, prolonging serum half-life through interaction with, e.g., FcRn, or while retaining its PK profile, but which has reduced or absent binding to one or more other Fc receptor(s), including without limitation a human FcγR as listed supra.

In some embodiments, the Fc region of an ABP provided herein is a human IgG4 Fc region comprising one or more of the hinge stabilizing mutations S228P and L235E, according to EU numbering. See Aalberse et al., Immunology, 2002, 105:9-19, incorporated by reference in its entirety. In some embodiments, the IgG4 Fc region comprises one or more of the following mutations: E233P, F234V, and L235A, according to EU numbering. See Armour et al., Mol. Immunol., 2003, 40:585-593, incorporated by reference in its entirety. In some embodiments, the IgG4 Fc region comprises a deletion at position G236.

In some embodiments, the Fc region of an ABP provided herein is a human IgG1 Fc region comprising one or more mutations to reduce Fc receptor binding. In some aspects, the one or more mutations are in residues selected from S228 (e.g., S228A), L234 (e.g., L234A), L235 (e.g., L235A), D265 (e.g., D265A), and N297 (e.g., N297A), according to EU numbering. In some aspects, the ABP comprises a PVA236 mutation. PVA236 means that the amino acid sequence ELLG (SEQ ID NO: 10), from amino acid position 233 to 236 of IgG1 or EFLG (SEQ ID NO: 11) of IgG4, is replaced by PVA, according to EU numbering. See U.S. Pat. No. 9,150,641, incorporated by reference in its entirety.

In some embodiments, the Fc region of an ABP provided herein is modified as described in Armour et al., Eur. J. Immunol., 1999, 29:2613-2624; WO 1999/058572; and/or U.K. Pat. App. No. 98099518; each of which is incorporated by reference in its entirety.

In some embodiments, the Fc region of an ABP provided herein is a human IgG2 Fc region comprising one or more of mutations A330S and P331S, according to EU numbering.

In some embodiments, the Fc region of an ABP provided herein has an amino acid substitution at one or more positions selected from 238, 265, 269, 270, 297, 327 and 329, according to EU numbering. See U.S. Pat. No. 6,737,056, incorporated by reference in its entirety. Such Fc mutants include Fc mutants with substitutions at two or more of amino acid positions 265, 269, 270, 297 and 327, including the so-called “DANA” Fc mutant with substitution of residues 265 and 297 with alanine, according to EU numbering. See U.S. Pat. No. 7,332,581, incorporated by reference in its entirety. In some embodiments, the ABP comprises an alanine at amino acid position 265. In some embodiments, the ABP comprises an alanine at amino acid position 297.

In certain embodiments, an ABP provided herein comprises an Fc region with one or more amino acid substitutions which improve ADCC, such as a substitution at one or more of positions 298, 333, and 334 of the Fc region, according to EU numbering. In some embodiments, an ABP provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety, according to EU numbering.

In some embodiments, an ABP provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

In some embodiments, an ABP provided herein comprises one or more alterations to increase half-life. ABPs with increased half-lives and improved binding to the neonatal Fc receptor (FcRn) are described, for example, in Hinton et al., J. Immunol., 2006, 176:346-356; and U.S. Pat. Pub. No. 2005/0014934; each of which is incorporated by reference in its entirety. Such Fc variants include those with substitutions at one or more of Fc region residues: 238, 250, 256, 265, 272, 286, 303, 305, 307, 311, 312, 314, 317, 340, 356, 360, 362, 376, 378, 380, 382, 413, 424, 428, and 434 of an IgG, according to EU numbering. In some embodiments, the ABP comprises one or more non-Fc modifications that extend half-life. Exemplary non-Fc modifications that extend half-life are described in, e.g., US20170218078, which is hereby incorporated by reference in its entirety.

In some embodiments, an ABP provided herein comprises a G1m17,1 allotype. Such allotype is described in, e.g., Lefranc G, Lefranc M-P. Gm allotype and Gm haplotypes>Allotypes. In IMGT Repertoire (IG and TR). IMGT®, the international ImMunoGeneTics information System®. http://http://www.imgt.org/IMGTrepertoire/Proteins/allotypes/human/IGH/IGHC/G1m_allotypes.html, which is hereby incorporated by reference in its entirety.

In some embodiments, an ABP provided herein comprises one or more Fc region variants as described in U.S. Pat. Nos. 7,371,826 5,648,260, and 5,624,821; Duncan and Winter, Nature, 1988, 322:738-740; and WO 94/29351; each of which is incorporated by reference in its entirety.

In some embodiments, the multispecific ABP comprises one or more Fc modifications that promote heteromultimerization. In some embodiments, the Fc modification comprises a knob-in-hole modification. Knob-in-hole modifications are described in, e.g., U.S. Pat. No. 7,695,936, Merchant et al., Nature Biotechnology 1998 July; 16(7):677-81; Ridgway et al., Protein Engineering 1996 July; 9(7):617-21; and Atwell et al., J Mol Biol. 1997 Jul. 4; 270(1):26-35, each of which is incorporated by reference in its entirety.

In some embodiments, the hinge region on the knob side has a C220 mutation, e.g., C220S, according to EU numbering. This C220S mutation is added in the antibody arm that does not have a light-chain because a free Cys can cause challenges with folding.

In some embodiments, one Fc-bearing chain of the multispecific ABP comprises a T366W mutation, and the other Fc-bearing chain of the multispecific ABP comprises a T366S, L368A, and Y407V mutation, according to EU numbering. In some embodiments, the multispecific ABP comprising a knob-in-hole modification further comprises an engineered disulfide bridge in the Fc region. In some embodiments, the engineered disulfide bridge comprises a K392C mutation in one Fc-bearing chain of the multispecific ABP, and a D399C in the other Fc-bearing chain of the multispecific ABP, according to EU numbering. In some embodiments, the engineered disulfide bridge comprises a S354C mutation in one Fc-bearing chain of the multispecific ABP, and a Y349C mutation in the other Fc-bearing chain of the multispecific ABP, according to EU numbering.

In some embodiments, the multispecific ABP comprises a T366W mutation, and the other Fc-bearing chain of the multispecific ABP comprises a T366S, L368A, and Y407V mutation, according to EU numbering; and the ABP comprises an engineered disulfide bridge, wherein the engineered disulfide bridge comprises a S354C mutation in one Fc-bearing chain of the multispecific ABP, and a Y349C mutation in the other Fc-bearing chain of the multispecific ABP, according to EU numbering.

In some embodiments, the engineered disulfide bridge comprises a 447C mutation in both Fc-bearing chains of the multispecific ABP, which 447C mutations are provided by extension of the C-terminus of a CH3 domain incorporating a KSC tripeptide sequence. In some embodiments, the multispecific ABP comprises an S354C and T366W mutation in one Fc-bearing chain and a Y349C, T366S, L368A and Y407V mutation in the other Fc-bearing chain, according to EU numbering.

In some embodiments, the Fc modification comprises a set of mutations described in Von Kreudenstein T S, Escobar-Carbrera E, Lario P I, et al. Improving biophysical properties of a bispecific antibody scaffold to aid developability: quality by molecular design. MAbs. 2013; 5(5):646-54, which is hereby incorporated by reference in its entirety. In some embodiments, the Fc modification comprises a set of mutations as provided in the following table (numbering is according to EU numbering).

| Chain-A | Chain-B |
|---|---|
| F405A_Y407V | T394W |
| F405A_Y407V | T366I_T394W |
| F405A_Y407V | T366L_T394W |
| F405A_Y407V | T366L_K392M_T394W |
| L351Y_F405A_Y407V | T366L_K392M_T394W |
| T350V_L351Y_F405A_Y407V | T350V_T366L_K392M_T394W |
| T350V_L351Y_F405A_Y407V | T350V_T366L_K392L_T394W |

In some embodiments, the Fc modification comprises a set of mutations described in Labrijn A F, et al., Proc Natl Acad Sci USA. 2013 Mar. 26; 110(13):5145-50. doi: 10.1073/pnas. In some embodiments, the Fc region is an IgG1 Fc, and the Fc modification comprises a K409R mutation in one Fc-bearing chain and a mutation selected from a Y407, L368, F405, K370, and D399 mutation in the other Fc-bearing chain, according to EU numbering. In some embodiments, the Fc modification comprises a K409R mutation in one Fc-bearing chain and a F405L mutation in the other Fc-bearing chain, according to EU numbering.

In some embodiments, the Fc modification comprises a set of mutations that renders homodimerization electrostatically unfavorable but heterodimerization favorable. An exemplary set of mutations is described in U.S. Pat. No. 8,592,562, and in Gunasekaran K et al., The Journal of Biological Chemistry 285, 19637-19646, doi: 10.1074/jbc.M110.117382, which are each incorporated by reference in its entirety. In some embodiments, the Fc modification comprises a K409D_K392D mutation in one Fc-bearing chain and a D399K_E356K mutation in the other Fc-bearing chain, according to EU numbering.

In some embodiments, the Fc modification comprises a set of mutations described in WO2011143545, which is hereby incorporated by reference in its entirety. In some embodiments, the Fc modification comprises a K409R mutation in one Fc-bearing chain and a L368E or L368D mutation in the other Fc-bearing chain, according to EU numbering. In some embodiments, the Fc modification comprises a set of mutations described in Strop P et al., J. Mol. Biol., 420 (2012), pp. 204-219, which is hereby incorporated by reference in its entirety. In some embodiments, the Fc modification comprises a D221E, P228E, and L368E mutation in one Fc-bearing chain and a D221R, P228R, and K409R in the other Fc-bearing chain, according to EU numbering.

In some embodiments, the Fc modification comprises a set of mutations described in Moore G L, et al., mAbs, 3 (2011), pp. 546-557, which is hereby incorporated by reference in its entirety. In some embodiments, the Fc modification comprises an S364H and F405A mutation in one Fc-bearing chain and a Y349T and T394F mutation in the other Fc-bearing chain, according to EU numbering. In some embodiments, the Fc modification comprises a set of mutations described in U.S. Pat. No. 9,822,186, which is hereby incorporated by reference in its entirety. In some embodiments, the Fc modification comprises an E375Q and S364K mutation in one Fc-bearing chain and a L368D and K370S mutation in the other Fc-bearing chain, according to EU numbering.

In some embodiments, the Fc modification comprises strand-exchange engineered domain (SEED) CH3 heterodimers. Such SEED CH3 heterodimers are described in, e.g., Davis J H et al., Protein Eng Des Sel. 2010 April; 23(4):195-202. doi: 10.1093/protein/gzp094, which is hereby incorporated by reference in its entirety.

In some embodiments, the Fc modification comprises a modification in the CH3 sequence that affects the ability of the CH3 domain to bind an affinity agent, e.g., Protein A. Such modifications, and methods of producing multispecific ABPs comprising the modifications, are described in U.S. Pat. No. 8,586,713, US20160024147A1, and Smith E J, et al., Scientific Reports 2015 Dec. 11; 5:17943. doi: 10.1038/srep17943., each of which is hereby incorporated by reference in its entirety. In some embodiments, the Fc modification comprises a H435 and/or Y436 mutation (e.g., H435R and/or Y436F mutation) in at least one Fc-bearing chain, according to EU numbering. In some embodiments, the other Fc-bearing chain does not comprise an amino acid mutation.

Antibodies Specific for B*35:01_EVDPIGHVY (SEQ ID NO: 9) (HLA-PEPTIDE Target “G5”)

In some aspects, provided herein are ABPs comprising antibodies or antigen-binding fragments thereof that specifically bind an HLA-PEPTIDE target, wherein the HLA Class I molecule of the HLA-PEPTIDE target is HLA subtype B*35:01 and the HLA-restricted peptide of the HLA-PEPTIDE target comprises, consists of, or essentially consists of the sequence EVDPIGHVY (SEQ ID NO: 9) (“G5”).

HLA-PEPTIDE target B*35:01_EVDPIGHVY (SEQ ID NO: 9) refers to an HLA-PEPTIDE target comprising the HLA-restricted peptide EVDPIGHVY (SEQ ID NO: 9) complexed with the HLA Class I molecule B*35:01, wherein the HLA-restricted peptide is located in the peptide binding groove of an α1/α2 heterodimer portion of the HLA Class I molecule. The restricted peptide is from tumor-specific gene product MAGEA6.

CDRs

The ABP specific for B*35:01_EVDPIGHVY (SEQ ID NO: 9) may comprise one or more antibody complementarity determining region (CDR) sequences, e.g., may comprise three heavy chain CDRs (CDR-H1, CDR-H2, CDR-H3) and three light chain CDRs (CDR-L1, CDR-L2, CDR-L3).

The ABP specific for B*35:01_EVDPIGHVY (SEQ ID NO: 9) may comprise a CDR-H3 sequence. The CDR-H3 sequence may be selected from CARDGVRYYGMDVW (SEQ ID NO: 67), CARGVRGYDRSAGYW (SEQ ID NO: 68), CASHDYGDYGEYFQHW (SEQ ID NO: 69), CARVSWYCSSTSCGVNWFDPW (SEQ ID NO: 70), CAKVNWNDGPYFDYW (SEQ ID NO: 71), CATPTNSGYYGPYYYYGMDVW (SEQ ID NO: 72), CARDVMDVW (SEQ ID NO: 73), CAREGYGMDVW (SEQ ID NO: 74), CARDNGVGVDYW (SEQ ID NO: 75), CARGIADSGSYYGNGRDYYYGMDVW (SEQ ID NO: 76), CARGDYYFDYW (SEQ ID NO: 77), CARDGTRYYGMDVW (SEQ ID NO: 78), CARDVVANFDYW (SEQ ID NO: 79), CARGHSSGWYYYYGMDVW (SEQ ID NO: 80), CAKDLGSYGGYYW (SEQ ID NO: 81), CARSWFGGFNYHYYGMDVW (SEQ ID NO: 82), CARELPIGYGMDVW (SEQ ID NO: 83), and CARGGSYYYYGMDVW (SEQ ID NO: 84).

The ABP specific for B*35:01_EVDPIGHVY (SEQ ID NO: 9) may comprise a CDR-L3 sequence. The CDR-L3 sequence may be selected from CMQGLQTPITF (SEQ ID NO: 85), CMQALQTPPTF (SEQ ID NO: 86), CQQAISFPLTF (SEQ ID NO: 87), CQQANSFPLTF (SEQ ID NO: 88), CQQANSFPLTF (SEQ ID NO: 88), CQQSYSIPLTF (SEQ ID NO: 59), CQQTYMMPYTF (SEQ ID NO: 89), CQQSYITPWTF (SEQ ID NO: 90), CQQSYITPYTF (SEQ ID NO: 91), CQQYYTTPYTF (SEQ ID NO: 92), CQQSYSTPLTF (SEQ ID NO: 55), CMQALQTPLTF (SEQ ID NO: 93), CQQYGSWPRTF (SEQ ID NO: 94), CQQSYSTPVTF (SEQ ID NO: 95), CMQALQTPYTF (SEQ ID NO: 96), CQQANSFPFTF (SEQ ID NO: 97), CMQALQTPLTF (SEQ ID NO: 93), and CQQSYSTPLTF (SEQ ID NO: 55).

The ABP specific for B*35:01_EVDPIGHVY (SEQ ID NO: 9) may comprise a particular heavy chain CDR3 (CDR-H3) sequence and a particular light chain CDR3 (CDR-L3) sequence. In some embodiments, the ABP comprises the CDR-H3 and the CDR-L3 from the scFv designated G5(7E07), G5(7B03), G5(7A05), G5(7F06), G5(1B12), G5(1C12), G5(1E05), G5(3G01), G5(3G08), G5(4B02), G5(4E04), G5(1D06), G5(1H11), G5(2B10), G5(2H08), G5(3G05), G5(4A07), or G5(4B01). CDR sequences of identified scFvs that specifically bind B*35:01_EVDPIGHVY (SEQ ID NO: 9) are shown in Table 5. For clarity, each identified scFv hit is designated a clone name, and each row contains the CDR sequences for that particular clone name. For example, the scFv identified by clone name G5(7E07) comprises the heavy chain CDR3 sequence CARDGVRYYGMDVW (SEQ ID NO: 67) and the light chain CDR3 sequence CMQGLQTPITF (SEQ ID NO: 85).

The ABP specific for B*35:01_EVDPIGHVY (SEQ ID NO: 9) may comprise all six CDRs from the scFv designated G5(7E07), G5(7B03), G5(7A05), G5(7F06), G5(1B12), G5(1C12), G5(1E05), G5(3G01), G5(3G08), G5(4B02), G5(4E04), G5(1D06), G5(1H11), G5(2B10), G5(2H08), G5(3G05), G5(4A07), or G5(4B01).

VH

The ABP specific for B*35:01_EVDPIGHVY (SEQ ID NO: 9) may comprise a VH sequence. The VH sequence may be selected from

(SEQ ID NO: 141)
QVQLVQSGAEVKKPGASVKVSCKASGYTFTSYDINWVRQA
PGQGLEWMGIINPRSGSTKYAQKFQGRVTMTRDTSTSTVY
MELSSLRSEDTAVYYCARDGVRYYGMDVWGQGTTVTVSS,
(SEQ ID NO: 142)
QVQLVQSGAEVKKPGSSVKVSCKASGYTFTSHDINWVRQA
PGQGLEWMGWMNPNSGDTGYAQKFQGRVTITADESTSTAY
MELSSLRSEDTAVYYCARGVRGYDRSAGYWGQGTLVIVSS,
(SEQ ID NO: 143)
EVQLLESGGGLVKPGGSLRLSCAASGFSFSSYWMSWVRQA
PGKGLEWISYISGDSGYTNYADSVKGRFTISRDDSKNTLY
LQMNSLKTEDTAVYYCASHDYGDYGEYFQHWGQGTLVTVS
S,
(SEQ ID NO: 144)
EVQLLQSGGGLVQPGGSLRLSCAASGFTFSNSDMNWVRQA
PGKGLEWVAYISSGSSTIYYADSVKGRFTISRDNSKNTLY
LQMNSLRAEDTAVYYCARVSWYCSSTSCGVNWFDPWGQGT
LVTVSS,
(SEQ ID NO: 145)
EVQLLESGGGLVQPGGSLRLSCAASGFTFSNSDMNWVRQA
PGKGLEWVASISSSGGYINYADSVKGRFTISRDNSKNTLY
LQMNSLRAEDTAVYYCAKVNWNDGPYFDYWGQGTLVTVSS,
(SEQ ID NO: 146)
QVQLVQSGAEVKKPGSSVKVSCKASGGTFSNFGVSWLRQA
PGQGLEWMGGIIPILGTANYAQKFQGRVTITADESTSTAY
MELSSLRSEDTAVYYCATPTNSGYYGPYYYYGMDVWGQGT
TVTVSS,
(SEQ ID NO: 147)
QVQLVQSGAEVKKPGASVKVSCKASGYTFTSYNMHWVRQA
PGQGLEWMGWINPNSGGTNYAQKFQGRVTMTRDTSTSTVY
MELSSLRSEDTAVYYCARDVMDVWGQGTTVTVSS,
(SEQ ID NO: 148)
QVQLVQSGAEVKKPGASVKVSCKASGGTFSGYLVSWVRQA
PGQGLEWMGWINPNSGGTNTAQKFQGRVTMTRDTSTSTVY
MELSSLRSEDTAVYYCAREGYGMDVWGQGTTVTVSS,
(SEQ ID NO: 149)
QVQLVQSGAEVKKPGASVKVSCKASGYIFRNYPMHWVRQA
PGQGLEWMGWINPDSGGTKYAQKFQGRVTMTRDTSTSTVY
MELSSLRSEDTAVYYCARDNGVGVDYWGQGTLVTVSS,
(SEQ ID NO: 150)
QVQLVQSGAEVKKPGASVKVSCKASGYTFTGYYMHWVRQA
PGQGLEWMGWMNPNIGNTGYAQKFQGRVTMTRDTSTSTVY
MELSSLRSEDTAVYYCARGIADSGSYYGNGRDYYYGMDVW
GQGTTVTVSS,
(SEQ ID NO: 151)
QVQLVQSGAEVKKPGASVKVSCKASGGTFSSYGISWVRQA
PGQGLEWMGWINPNSGVTKYAQKFQGRVTMTRDTSTSTVY
MELSSLRSEDTAVYYCARGDYYFDYWGQGTLVTVSS,
(SEQ ID NO: 152)
QVQLVQSGAEVKKPGASVKVSCKASGYTFTSYDINWVRQA
PGQGLEWMGWINPNSGDTKYSQKFQGRVTMTRDTSTSTVY
MELSSLRSEDTAVYYCARDGTRYYGMDVWGQGTTVTVSS,
(SEQ ID NO: 153)
EVQLLESGGGLVKPGGSLRLSCAASGFTFSDYYMSWVRQA
PGKGLEWVSYISSSSSYTNYADSVKGRFTISRDDSKNTLY
LQMNSLKTEDTAVYYCARDVVANFDYWGQGTLVTVSS,
(SEQ ID NO: 154)
QVQLVQSGAEVKKPGASVKVSCKASGGTFSSYAISWVRQA
PGQGLEWMGWMNPDSGSTGYAQRFQGRVTMTRDTSTSTVY
MELSSLRSEDTAVYYCARGHSSGWYYYYGMDVWGQGTTVT
VSS,
(SEQ ID NO: 155)
EVQLLESGGGLVQPGGSLRLSCAASGFTFTSYSMHWVRQA
PGKGLEWVSSITSFTNTMYYADSVKGRFTISRDNSKNTLY
LQMNSLRAEDTAVYYCAKDLGSYGGYYWGQGTLVTVSS,
(SEQ ID NO: 156)
QVQLVQSGAEVKKPGASVKVSCKASGYTFTNYYMHWVRQA
PGQGLEWMGIINPSGGSTSYAQKFQGRVTMTRDTSTSTVY
MELSSLRSEDTAVYYCARSWFGGFNYHYYGMDVWGQGTTV
TVSS,
(SEQ ID NO: 157)
QVQLVQSGAEVKKPGASVKVSCKASGYTFTSYYMHWVRQA
PGQGLEWMGWMNPNSGNTGYAQKFQGRVTMTRDTSTSTVY
MELSSLRSEDTAVYYCARELPIGYGMDVWGQGTTVTVSS,
and
(SEQ ID NO: 158)
QVQLVQSGAEVKKPGSSVKVSCKASGGTFSSYAISWVRQA
PGQGLEWMGGIIPIVGTANYAQKFQGRVTITADESTSTAY
MELSSLRSEDTAVYYCARGGSYYYYGMDVWGQGTTVTVSS.

VL

The ABP specific for B*35:01_EVDPIGHVY (SEQ ID NO: 9) may comprise a VL sequence. The VL sequence may be selected from

(SEQ ID NO: 159)
DIVMTQSPLSLPVTPGEPASISCRSSQSLLHSNGYNYLDW
YLQKPGQSPQLLIYLGSYRASGVPDRFSGSGSGTDFTLKI
SRVEAEDVGVYYCMQGLQTPITFGQGTRLEIK,
(SEQ ID NO: 160)
DIVMTQSPLSLPVTPGEPASISCRSSQSLLHSNGYNYLDW
YLQKPGQSPQLLIYLGSSRASGVPDRFSGSGSGTDFTLKI
SRVEAEDVGVYYCMQALQTPPTFGPGTKVDIK,
(SEQ ID NO: 161)
DIQMTQSPSSLSASVGDRVTITCQASQDISNYLNWYQQKP
GKAPKLLIYAASSLQSGVPSRFSGSGSGTDFTLTISSLQP
EDFATYYCQQAISFPLTFGQSTKVEIK,
(SEQ ID NO: 162)
DIQMTQSPSSLSASVGDRVTITCRASQSISSWLAWYQQKP
GKAPKLLIYSASTLQSGVPSRFSGSGSGTDFTLTISSLQP
EDFATYYCQQANSFPLTFGGGTKVEIK,
(SEQ ID NO: 163)
DIQMTQSPSSLSASVGDRVTITCRASQSISSWLAWYQQKP
GKAPKLLIYAASSLQSGVPSRFSGSGSGTDFTLTISSLQP
EDFATYYCQQANSFPLTFGGGTKVEIK,
(SEQ ID NO: 164)
DIQMTQSPSSLSASVGDRVTITCRASQSISSWLAWYQQKP
GKAPKLLIYAASTLQSGVPSRFSGSGSGTDFTLTISSLQP
EDFATYYCQQSYSIPLTFGGGTKVEIK,
(SEQ ID NO: 165)
DIQMTQSPSSLSASVGDRVTITCRASQGISNYLNWYQQKP
GKAPKLLIYYASSLQSGVPSRFSGSGSGTDFTLTISSLQP
EDFATYYCQQTYMMPYTFGQGTKVEIK,
(SEQ ID NO: 166)
DIQMTQSPSSLSASVGDRVTITCRASQSISSYLNWYQQKP
GKAPKLLIYGASSLQSGVPSRFSGSGSGTDFTLTISSLQP
EDFATYYCQQSYITPWTFGQGTKVEIK,
(SEQ ID NO: 167)
DIQMTQSPSSLSASVGDRVTITCRASQGISNYLAWYQQKP
GKAPKLLIYAASSLQSGVPSRFSGSGSGTDFTLTISSLQP
EDFATYYCQQSYITPYTFGQGTKLEIK,
(SEQ ID NO: 168)
DIVMTQSPDSLAVSLGERATINCKTSQSVLYRPNNENYLA
WYQQKPGQPPKLLIYQASIREPGVPDRFSGSGSGTDFTLT
ISSLQAEDVAVYYCQQYYTTPYTFGQGTKLEIK
(SEQ ID NO: 169)
DIQMTQSPSSLSASVGDRVTITCRASQSISRFLNWYQQKP
GKAPKLLIYGASRPQSGVPSRFSGSGSGTDFTLTISSLQP
EDFATYYCQQSYSTPLTFGQGTKVEIK,
(SEQ ID NO: 170)
DIVMTQSPLSLPVTPGEPASISCRSSQSLLHSNGYNYLDW
YLQKPGQSPQLLIYLGSHRASGVPDRFSGSGSGTDFTLKI
SRVEAEDVGVYYCMQALQTPLTFGGGTKVEIK
(SEQ ID NO: 171)
EIVMTQSPATLSVSPGERATLSCRASQSVSSNLAWYQQKP
GQAPRLLIYAASARASGIPARFSGSGSGTEFTLTISSLQS
EDFAVYYCQQYGSWPRTFGQGTKVEIK,
(SEQ ID NO: 172)
DIQMTQSPSSLSASVGDRVTITCRASQSISSYLNWYQQKP
GKAPKLLIYGASRLQSGVPSRFSGSGSGTDFTLTISSLQP
EDFATYYCQQSYSTPVTFGQGTKVEIK,
(SEQ ID NO: 173)
DIVMTQSPLSLPVTPGEPASISCRSSQSLLHSNGYNYLDW
YLQKPGQSPQLLIYLGSNRASGVPDRFSGSGSGTDFTLKI
SRVEAEDVGVYYCMQALQTPYTFGQGTKVEIK
(SEQ ID NO: 174)
DIQMTQSPSSLSASVGDRVTITCQASEDISNHLNWYQQKP
GKAPKLLIYDALSLQSGVPSRFSGSGSGTDFTLTISSLQP
EDFATYYCQQANSFPFTFGPGTKVDIK,
(SEQ ID NO: 175)
DIVMTQSPLSLPVTPGEPASISCRSSQSLLHSNGYNYLDW
YLQKPGQSPQLLIYLGSNRASGVPDRFSGSGSGTDFTLKI
SRVEAEDVGVYYCMQALQTPLTFGQGTKVEIK
and
(SEQ ID NO: 176)
DIQMTQSPSSLSASVGDRVTITCRASQSISSYLNWYQQKP
GKAPKLLIYAASSLQSGVPSRFSGSGSGTDFTLTISSLQP
EDFATYYCQQSYSTPLTFGGGTKVEIK.

VH-VL Combinations

The ABP specific for B*35:01_EVDPIGHVY (SEQ ID NO: 9) may comprise a particular VH sequence and a particular VL sequence. In some embodiments, the ABP specific for B*35:01_EVDPIGHVY (SEQ ID NO: 9) comprises a VH sequence and VL sequence from the scFv designated G5(7E07), G5(7B03), G5(7A05), G5(7F06), G5(1B12), G5(1C12), G5(1E05), G5(3G01), G5(3G08), G5(4B02), G5(4E04), G5(1D06), G5(1H11), G5(2B10), G5(2H08), G5(3G05), G5(4A07), or G5(4B01). The VH and VL sequences of identified scFvs that specifically bind B*35:01_EVDPIGHVY (SEQ ID NO: 9) are shown in Table 4. For clarity, each identified scFv hit is designated a clone name, and each row contains the VH and VL sequences for that particular clone name. For example, the scFv identified by clone name G5(7E07) comprises the VH sequence

(SEQ ID NO: 141)
QVQLVQSGAEVKKPGASVKVSCKASGYTFTSYDINWVRQA
PGQGLEWMGIINPRSGSTKYAQKFQGRVTMTRDTSTSTVY
MELSSLRSEDTAVYYCARDGVRYYGMDVWGQGTTVTVSS
and
the VL sequence
(SEQ ID NO: 159)
DIVMTQSPLSLPVTPGEPASISCRSSQSLLHSNGYNYLDW
YLQKPGQSPQLLIYLGSYRASGVPDRFSGSGSGTDFTLKI
SRVEAEDVGVYYCMQGLQTPITFGQGTRLEIK.

Antibodies Specific for A*02:01_AIFPGAVPAA (SEQ ID NO: 6) (HLA-PEPTIDE Target “G8”)

In some aspects, provided herein are ABPs comprising antibodies or antigen-binding fragments thereof that specifically bind an HLA-PEPTIDE target, wherein the HLA Class I molecule of the HLA-PEPTIDE target is HLA subtype A*02:01 and the HLA-restricted peptide of the HLA-PEPTIDE target comprises, consists of, or essentially consists of the sequence AIFPGAVPAA (SEQ ID NO: 6) (“G8”).

HLA-PEPTIDE target A*02:01_AIFPGAVPAA (SEQ ID NO: 6), disclosed as Target #24053 in Table A, refers to an HLA-PEPTIDE target comprising the HLA-restricted peptide AIFPGAVPAA (SEQ ID NO: 6) complexed with the HLA Class I molecule A*02:01, wherein the HLA-restricted peptide is located in the peptide binding groove of an α1/α2 heterodimer portion of the HLA Class I molecule. The restricted peptide is from tumor-specific gene product FOXE1.

CDRs

The ABP specific for A*02:01_AIFPGAVPAA (SEQ ID NO: 6) may comprise one or more antibody complementarity determining region (CDR) sequences, e.g., may comprise three heavy chain CDRs (CDR-H1, CDR-H2, CDR-H3) and three light chain CDRs (CDR-L1, CDR-L2, CDR-L3).

The ABP specific for A*02:01_AIFPGAVPAA (SEQ ID NO: 6) may comprise a CDR-H3 sequence. The CDR-H3 sequence may be selected from

(SEQ ID NO: 177)
CARDDYGDYVAYFQHW,
(SEQ ID NO: 178)
CARDLSYYYGMDVW,
(SEQ ID NO: 179)
CARVYDFWSVLSGFDIW,
(SEQ ID NO: 180)
CARVEQGYDIYYYYYMDVW,
(SEQ ID NO: 181)
CARSYDYGDYLNFDYW,
(SEQ ID NO: 182)
CARASGSGYYYYYGMDVW,
(SEQ ID NO: 183)
CAASTWIQPFDYW,
(SEQ ID NO: 184)
CASNGNYYGSGSYYNYW,
(SEQ ID NO: 185)
CARAVYYDFWSGPFDYW,
(SEQ ID NO: 186)
CAKGGIYYGSGSYPSW,
(SEQ ID NO: 187)
CARGLYYMDVW,
(SEQ ID NO: 188)
CARGLYGDYFLYYGMDVW,
(SEQ ID NO: 189)
CARGLLGFGEFLTYGMDVW,
(SEQ ID NO: 190)
CARDRDSSWTYYYYGMDVW,
(SEQ ID NO: 188)
CARGLYGDYFLYYGMDVW,
(SEQ ID NO: 191)
CARGDYYDSSGYYFPVYFDYW,
and
(SEQ ID NO: 192)
CAKDPFWSGHYYYYGMDVW.

The ABP specific for A*02:01_AIFPGAVPAA (SEQ ID NO: 6) may comprise a CDR-L3 sequence. The CDR-L3 sequence may be selected from CQQNYNSVTF (SEQ ID NO: 194), CQQSYNTPWTF (SEQ ID NO: 195), CGQSYSTPPTF (SEQ ID NO: 196), CQQSYSAPYTF (SEQ ID NO: 49), CQQSYSIPPTF (SEQ ID NO: 197), CQQSYSAPYTF (SEQ ID NO: 49), CQQHNSYPPTF (SEQ ID NO: 198), CQQYSTYPITI (SEQ ID NO: 199), CQQANSFPWTF (SEQ ID NO: 200), CQQSHSTPQTF (SEQ ID NO: 201), CQQSYSTPLTF (SEQ ID NO: 55), CQQSYSTPLTF (SEQ ID NO: 55), CQQTYSTPWTF (SEQ ID NO: 202), CQQYGSSPYTF (SEQ ID NO: 203), CQQSHSTPLTF (SEQ ID NO: 204), CQQANGFPLTF (SEQ ID NO: 205), and CQQSYSTPLTF (SEQ ID NO: 55).

The ABP specific for A*02:01_AIFPGAVPAA (SEQ ID NO: 6) may comprise a particular heavy chain CDR3 (CDR-H3) sequence and a particular light chain CDR3 (CDR-L3) sequence. In some embodiments, the ABP comprises the CDR-H3 and the CDR-L3 from the scFv designated G8(1A03), G8(1A04), G8(1A06), G8(1B03), G8(1C11), G8(1D02), G8(1H08), G8(2B05), G8(2E06), G8(2C10), G8(2E04), G8(4F05), G8(5C03), G8(5F02), G8(5G08), G8(1C01), or G8(2C11). CDR sequences of identified scFvs that specifically bind A*02:01_AIFPGAVPAA (SEQ ID NO: 6) are shown in Table 7. For clarity, each identified scFv hit is designated a clone name, and each row contains the CDR sequences for that particular clone name. For example, the scFv identified by clone name G8(1A03) comprises the heavy chain CDR3 sequence CARDDYGDYVAYFQHW (SEQ ID NO: 177) and the light chain CDR3 sequence CQQNYNSVTF (SEQ ID NO: 194).

The ABP specific for A*02:01_AIFPGAVPAA (SEQ ID NO: 6) may comprise all six CDRs from the scFv designated G8(1A03), G8(1A04), G8(1A06), G8(1B03), G8(1C11), G8(1D02), G8(1H08), G8(2B05), G8(2E06), G8(2C10), G8(2E04), G8(4F05), G8(5C03), G8(5F02), G8(5G08), G8(1C01), or G8(2C11).

VH

The ABP specific for A*02:01_AIFPGAVPAA (SEQ ID NO: 6) may comprise a VH sequence. The VH sequence may be selected from

(SEQ ID NO: 206)
QVQLVQSGAEVKKPGASVKVSCKASGGTFSRSAITWVRQA
PGQGLEWMGWINPNSGATNYAQKFQGRVTMTRDTSTSTVY
MELSSLRSEDTAVYYCARDDYGDYVAYFQHWGQGTLVTVS
S,
(SEQ ID NO: 207)
QVQLVQSGAEVKKPGASVKVSCKASGYPFIGQYLHWVRQA
PGQGLEWMGIINPSGDSATYAQKFQGRVTMTRDTSTSTVY
MELSSLRSEDTAVYYCARDLSYYYGMDVWGQGTTVTVSS,
(SEQ ID NO: 208)
QVQLVQSGAEVKKPGASVKVSCKASGYTFTNYYMHWVRQA
PGQGLEWMGWMNPIGGGTGYAQKFQGRVTMTRDTSTSTVY
MELSSLRSEDTAVYYCARVYDFWSVLSGFDIWGQGTLVTV
SS,
(SEQ ID NO: 209)
EVQLLESGGGLVQPGGSLRLSCAASGFTFSDYYMSWVRQA
PGKGLEWVSGINWNGGSTGYADSVKGRFTISRDNSKNTLY
LQMNSLRAEDTAVYYCARVEQGYDIYYYYYMDVWGKGTTV
TVSS,
(SEQ ID NO: 210)
QVQLVQSGAEVKKPGASVKVSCKASGGTLSSYPINWVRQA
PGQGLEWMGWISTYSGHADYAQKLQGRVTMTRDTSTSTVY
MELSSLRSEDTAVYYCARSYDYGDYLNFDYWGQGTLVTVS
S,
(SEQ ID NO: 211)
EVQLLESGGGLVQPGGSLRLSCAASGFTFSSYWMSWVRQA
PGKGLEWVSSISGRGDNTYYADSVKGRFTISRDNSKNTLY
LQMNSLRAEDTAVYYCARASGSGYYYYYGMDVWGQGTTVT
VSS,
(SEQ ID NO: 212)
QVQLVQSGAEVKKPGASVKVSCKASGYTFGNYFMHWVRQA
PGQGLEWMGMVNPSGGSETFAQKFQGRVTMTRDTSTSTVY
MELSSLRSEDTAVYYCAASTWIQPFDYWGQGTLVTVSS,
(SEQ ID NO: 213)
EVQLLESGGGLVQPGGSLRLSCAASGFDFSIYSMNWVRQA
PGKGLEWVSAISGSGGSTYYADSVKGRFTISRDNSKNTLY
LQMNSLRAEDTAVYYCASNGNYYGSGSYYNYWGQGTLVTV
SS,
(SEQ ID NO: 214)
QVQLVQSGAEVKKPGASVKVSCKASGYTLTTYYMHWVRQA
PGQGLEWMGWINPNSGGTNYAQKFQGRVTMTRDTSTSTVY
MELSSLRSEDTAVYYCARAVYYDFWSGPFDYWGQGTLVTV
SS,
(SEQ ID NO: 215)
QVQLVQSGAEVKKPGASVKVSCKASGYTFTSYYMHWVRQA
PGQGLEWMGWINPYSGGTNYAQKFQGRVTMTRDTSTSTVY
MELSSLRSEDTAVYYCAKGGIYYGSGSYPSWGQGTLVTVS
S,
(SEQ ID NO: 216)
QVQLVQSGAEVKKPGSSVKVSCKASGGTFSSYGVSWVRQA
PGQGLEWMGWISPYSGNTDYAQKFQGRVTITADESTSTAY
MELSSLRSEDTAVYYCARGLYYMDVWGKGTTVTVSS,
(SEQ ID NO: 217)
QVQLVQSGAEVKKPGASVKVSCKASGYTFSNMYLHWVRQA
PGQGLEWMGWINPNTGDTNYAQTFQGRVTMTRDTSTSTVY
MELSSLRSEDTAVYYCARGLYGDYFLYYGMDVWGQGTKVT
VSS,
(SEQ ID NO: 218)
QVQLVQSGAEVKKPGASVKVSCKASGYTFTSYYMHWVRQA
PGQGLEWMGWMNPNSGNTGYAQKFQGRVTMTRDTSTSTVY
MELSSLRSEDTAVYYCARGLLGFGEFLTYGMDVWGQGTLV
TVSS,
(SEQ ID NO: 219)
QVQLVQSGAEVKKPGASVKVSCKASGYTFTGYYIHWVRQA
PGQGLEWMGVINPSGGSTTYAQKLQGRVTMTRDTSTSTVY
MELSSLRSEDTAVYYCARDRDSSWTYYYYGMDVWGQGTTV
TVSS,
(SEQ ID NO: 220)
QVQLVQSGAEVKKPGASVKVSCKASGYTFTSNYMHWVRQA
PGQGLEWMGWMNPNSGNTGYAQKFQGRVTMTRDTSTSTVY
MELSSLRSEDTAVYYCARGLYGDYFLYYGMDVWGQGTTVT
VSS,
(SEQ ID NO: 221)
QVQLVQSGAEVKKPGASVKVSCKASGGTFSSHAISWVRQA
PGQGLEWMGVIIPSGGTSYTQKFQGRVTMTRDTSTSTVYM
ELSSLRSEDTAVYYCARGDYYDSSGYYFPVYFDYWGQGTL
VTVSS,
and
(SEQ ID NO: 222)
QVQLVQSGAEVKKPGASVKVSCKASGYTFTSYAMNWVRQA
PGQGLEWMGWINPNSGGTNYAQKFQGRVTMTRDTSTSTVY
MELSSLRSEDTAVYYCARDPFWSGHYYYYGMDVWGQGTTV
TVSS.

VL

The ABP specific for A*02:01_AIFPGAVPAA (SEQ ID NO: 6) may comprise a VL sequence. The VL sequence may be selected from

(SEQ ID NO: 223)
DIQMTQSPSSLSASVGDRVTITCRASQSITSYLNWYQQKPGKAPKLLIY
DASNLETGVPSRFSGSGSGTDFTLTISSLQPEDFATYYCQQNYNSVTFG
QGTKLEIK,
(SEQ ID NO: 224)
DIQMTQSPSSLSASVGDRVTITCWASQGISSYLAWYQQKPGKAPKLLIY
AASSLQSGVPSRFSGSGSGTDFTLTISSLQPEDFATYYCQQSYNTPWTF
GPGTKVDIK,
(SEQ ID NO: 225)
DIQMTQSPSSLSASVGDRVTITCRASQAISNSLAWYQQKPGKAPKLLIY
AASTLQSGVPSRFSGSGSGTDFTLTISSLQPEDFATYYCGQSYSTPPTF
GQGTKLEIK,
(SEQ ID NO: 226)
DIQMTQSPSSLSASVGDRVTITCRASQSISSYLNWYQQKPGKAPKLLIY
KASSLESGVPSRFSGSGSGTDFTLTISSLQPEDFATYYCQQSYSAPYTF
GPGTKVDIK,
(SEQ ID NO: 227)
DIQMTQSPSSLSASVGDRVTITCQASQDISNYLNWYQQKPGKAPKLLIY
AASSLQSGVPSRFSGSGSGTDFTLTISSLQPEDFATYYCQQSYSIPPTF
GGGTKVDIK,
(SEQ ID NO: 228)
DIQMTQSPSSLSASVGDRVTITCQASQDISNYLNWYQQKPGKAPKLLIY
AASSLQSGVPSRFSGSGSGTDFTLTISSLQPEDFATYYCQQSYSAPYTF
GGGTKVEIK,
(SEQ ID NO: 229)
DIQMTQSPSSLSASVGDRVTITCRASQGINSYLAWYQQKPGKAPKLLIY
DASNLETGVPSRFSGSGSGTDFTLTISSLQPEDFATYYCQQHNSYPPTF
GQGTKLEIK,
(SEQ ID NO: 230)
DIQMTQSPSSLSASVGDRVTITCRASQSISRWLAWYQQKPGKAPKLLIY
AASSLQSGVPSRFSGSGSGTDFTLTISSLQPEDFATYYCQQYSTYPITI
GQGTKVEIK,
(SEQ ID NO: 231)
DIQMTQSPSSLSASVGDRVTITCRASQGISNSLAWYQQKPGKAPKLLIY
AASSLQSGVPSRFSGSGSGTDFTLTISSLQPEDFATYYCQQANSFPWTF
GQGTKLEIK,
(SEQ ID NO: 232)
DIQMTQSPSSLSASVGDRVTITCRASQDVSTWLAWYQQKPGKAPKLLIY
AASSLQSGVPSRFSGSGSGTDFTLTISSLQPEDFATYYCQQSHSTPQTF
GQGTKVEIK,
(SEQ ID NO: 233)
DIQMTQSPSSLSASVGDRVTITCRASQSISSWLAWYQQKPGKAPKLLIY
DASNLETGVPSRFSGSGSGTDFTLTISSLQPEDFATYYCQQSYSTPLTF
GGGTKLEIK,
(SEQ ID NO: 234)
DIQMTQSPSSLSASVGDRVTITCRASQGISNYLAWYQQKPGKAPKLLIY
AASTLQSGVPSRFSGSGSGTDFTLTISSLQPEDFATYYCQQSYSTPLTF
GGGTKVEIK,
(SEQ ID NO: 235)
DIQMTQSPSSLSASVGDRVTITCRASQGISNWLAWYQQKPGKAPKLLIY
AASTLQSGVPSRFSGSGSGTDFTLTISSLQPEDFATYYCQQTYSTPWTF
GQGTKLEIK,
(SEQ ID NO: 236)
EIVMTQSPATLSVSPGERATLSCRASQSVGNSLAWYQQKPGQAPRLLIY
GASTRATGIPARFSGSGSGTEFTLTISSLQSEDFAVYYCQQYGSSPYTF
GQGTKVEIK,
(SEQ ID NO: 237)
DIQMTQSPSSLSASVGDRVTITCRASQSISGYLNWYQQKPGKAPKLLIY
AASSLQSGVPSRFSGSGSGTDFTLTISSLQPEDFATYYCQQSHSTPLTF
GQGTKVEIK,
(SEQ ID NO: 238)
DIQMTQSPSSLSASVGDRVTITCRASQNIYTYLNWYQQKPGKAPKLLIY
DASNLETGVPSRFSGSGSGTDFTLTISSLQPEDFATYYCQQANGFPLTF
GGGTKVEIK,
and
(SEQ ID NO: 176)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIK.

VH-VL Combinations

The ABP specific for A*02:01_AIFPGAVPAA (SEQ ID NO: 6) may comprise a particular VH sequence and a particular VL sequence. In some embodiments, the ABP specific for A*02:01_AIFPGAVPAA (SEQ ID NO: 6) comprises a VH sequence and VL sequence from the scFv designated G8(1A03), G8(1A04), G8(1A06), G8(1B03), G8(1C11), G8(1D02), G8(1H08), G8(2B05), G8(2E06), G8(2C10), G8(2E04), G8(4F05), G8(5C03), G8(5F02), G8(5G08), G8(1C01), or G8(2C11). The VH and VL sequences of identified scFvs that specifically bind A*02:01_AIFPGAVPAA (SEQ ID NO: 6) are shown in Table 6. For clarity, each identified scFv hit is designated a clone name, and each row contains the VH and VL sequences for that particular clone name. For example, the scFv identified by clone name G8(1A03) comprises the VH sequence

(SEQ ID NO: 206)
QVQLVQSGAEVKKPGASVKVSCKASGGTFSRSAITWVRQAPGQGLEWMG
WINPNSGATNYAQKFQGRVTMTRDTSTSTVYMELSSLRSEDTAVYYCAR
DDYGDYVAYFQHWGQGTLVTVSS
and
the VL sequence
(SEQ ID NO: 223)
DIQMTQSPSSLSASVGDRVTITCRASQSITSYLNWYQQKPGKAPKLLIY
DASNLETGVPSRFSGSGSGTDFTLTISSLQPEDFATYYCQQNYNSVTFG
QGTKLEIK.

Antibodies Specific for A*01:01_ASSLPTTMNY (SEQ ID NO: 7) (HLA-PEPTIDE Target “G10”)

In some aspects, provided herein are ABPs comprising antibodies or antigen-binding fragments thereof that specifically bind an HLA-PEPTIDE target, wherein the HLA Class I molecule of the HLA-PEPTIDE target is HLA subtype A*01:01 and the HLA-restricted peptide of the HLA-PEPTIDE target comprises, consists of, or essentially consists of the sequence ASSLPTTMNY (SEQ ID NO: 7) (“G10”).

HLA-PEPTIDE target A*01:01_ASSLPTTMNY (SEQ ID NO: 7), disclosed as Target #39108 in Table A, refers to an HLA-PEPTIDE target comprising the HLA-restricted peptide ASSLPTTMNY (SEQ ID NO: 7) complexed with the HLA Class I molecule A*01:01, wherein the HLA-restricted peptide is located in the peptide binding groove of an α1/α2 heterodimer portion of the HLA Class I molecule. The restricted peptide is from tumor-specific gene products MAGEA3 and MAGEA6.

CDRs

The ABP specific for A*01:01_ASSLPTTMNY (SEQ ID NO: 7) may comprise one or more antibody complementarity determining region (CDR) sequences, e.g., may comprise three heavy chain CDRs (CDR-H1, CDR-H2, CDR-H3) and three light chain CDRs (CDR-L1, CDR-L2, CDR-L3).

The ABP specific for A*01:01_ASSLPTTMNY (SEQ ID NO: 7) may comprise a CDR-H3 sequence. The CDR-H3 sequence may be selected from

(SEQ ID NO: 239)
CARDQDTIFGVVITWFDPW,
(SEQ ID NO: 240)
CARDKVYGDGFDPW,
(SEQ ID NO: 241)
CAREDDSMDVW,
(SEQ ID NO: 242)
CARDSSGLDPW,
(SEQ ID NO: 243)
CARGVGNLDYW,
(SEQ ID NO: 244)
CARDAHQYYDFWSGYYSGTYYYGMDVW,
(SEQ ID NO: 245)
CAREQWPSYWYFDLW,
(SEQ ID NO: 246)
CARDRGYSYGYFDYW,
(SEQ ID NO: 247)
CARGSGDPNYYYYYGLDVW,
(SEQ ID NO: 248)
CARDTGDHFDYW,
(SEQ ID NO: 249)
CARAENGMDVW,
(SEQ ID NO: 250)
CARDPGGYMDVW,
(SEQ ID NO: 251)
CARDGDAFDIW,
(SEQ ID NO: 252)
CARDMGDAFDIW,
(SEQ ID NO: 253)
CAREEDGMDVW,
(SEQ ID NO: 248)
CARDTGDHFDYW,
(SEQ ID NO: 254)
CARGEYSSGFFFVGWFDLW,
and
(SEQ ID NO: 255)
CARETGDDAFDIW.

The ABP specific for A*01:01_ASSLPTTMNY (SEQ ID NO: 7) may comprise a CDR-L3 sequence. The CDR-L3 sequence may be selected from CQQYFTTPYTF (SEQ ID NO: 256), CQQAEAFPYTF (SEQ ID NO: 257), CQQSYSTPITF (SEQ ID NO: 258), CQQSYIIPYTF (SEQ ID NO: 259), CHQTYSTPLTF (SEQ ID NO: 260), CQQAYSFPWTF (SEQ ID NO: 261), CQQGYSTPLTF (SEQ ID NO: 262), CQQANSFPRTF (SEQ ID NO: 263), CQQANSLPYTF (SEQ ID NO: 264), CQQSYSTPFTF (SEQ ID NO: 47), CQQSYSTPFTF (SEQ ID NO: 47), CQQSYGVPTF (SEQ ID NO: 265), CQQSYSTPLTF (SEQ ID NO: 55), CQQSYSTPLTF (SEQ ID NO: 55), CQQYYSYPWTF (SEQ ID NO: 266), CQQSYSTPFTF (SEQ ID NO: 47), CMQTLKTPLSF (SEQ ID NO: 267), and CQQSYSTPLTF (SEQ ID NO: 55).

The ABP specific for A*01:01_ASSLPTTMNY (SEQ ID NO: 7) may comprise a particular heavy chain CDR3 (CDR-H3) sequence and a particular light chain CDR3 (CDR-L3) sequence. In some embodiments, the ABP comprises the CDR-H3 and the CDR-L3 from the scFv designated G10(1A07), G10(1B07), G10(1E12), G10(1F06), G10(1H01), G10(1H08), G10(2C04), G10(2G11), G10(3E04), G10(4A02), G10(4C05), G10(4D04), G10(4D10), G10(4E07), G10(4E12), G10(4G06), G10(5A08), or G10(5C08). CDR sequences of identified scFvs that specifically bind A*01:01_ASSLPTTMNY (SEQ ID NO: 7) are shown in Table 9. For clarity, each identified scFv hit is designated a clone name, and each row contains the CDR sequences for that particular clone name. For example, the scFv identified by clone name G10(1A07) comprises the heavy chain CDR3 sequence CARDQDTIFGVVITWFDPW (SEQ ID NO: 239) and the light chain CDR3 sequence CQQYFTTPYTF (SEQ ID NO: 256).

The ABP specific for A*01:01_ASSLPTTMNY (SEQ ID NO: 7) may comprise all six CDRs from the scFv designated G10(1A07), G10(1B07), G10(1E12), G10(1F06), G10(1H01), G10(1H08), G10(2C04), G10(2G11), G10(3E04), G10(4A02), G10(4C05), G10(4D04), G10(4D10), G10(4E07), G10(4E12), G10(4G06), G10(5A08), or G10(5C08).

VH

The ABP specific for A*01:01_ASSLPTTMNY (SEQ ID NO: 7) may comprise a VH sequence. The VH sequence may be selected from

(SEQ ID NO: 268)
EVQLLESGGGLVKPGGSLRLSCAASGFTFSSYWMSWVRQAPGKGLEWVS
GISARSGRTYYADSVKGRFTISRDDSKNTLYLQMNSLKTEDTAVYYCAR
DQDTIFGVVITWFDPWGQGTLVTVSS,
(SEQ ID NO: 269)
QVQLVQSGAEVKKPGASVKVSCKASGYTFTSYYMHWVRQAPGQGLEWMG
IIHPGGGTTSYAQKFQGRVTMTRDTSTSTVYMELSSLRSEDTAVYYCAR
DKVYGDGFDPWGQGTLVTVSS,
(SEQ ID NO: 270)
QVQLVQSGAEVKKPGASVKVSCKASGYIFTGYYMHWVRQAPGQGLEWMG
MIGPSDGSTSYAQKFQGRVTMTRDTSTSTVYMELSSLRSEDTAVYYCAR
EDDSMDVWGKGTTVTVSS,
(SEQ ID NO: 271)
QVQLVQSGAEVKKPGASVKVSCKASGYTFIGYYMHWVRQAPGQGLEWMG
MIGPSDGSTSYAQKFQGRVTMTRDTSTSTVYMELSSLRSEDTAVYYCAR
DSSGLDPWGQGTLVTVSS,
(SEQ ID NO: 272)
QVQLVQSGAEVKKPGASVKVSCKASGYTFTGYYMHWVRQAPGQGLEWMG
MIGPSDGSTSYAQKFQGRVTMTRDTSTSTVYMELSSLRSEDTAVYYCAR
GVGNLDYWGQGTLVTVSS,
(SEQ ID NO: 273)
QVQLVQSGAEVKKPGASVKVSCKASGVTFSTSAISWVRQAPGQGLEWMG
WISPYNGNTDYAQMLQGRVTMTRDTSTSTVYMELSSLRSEDTAVYYCAR
DAHQYYDFWSGYYSGTYYYGMDVWGQGTTVTVSS,
(SEQ ID NO: 274)
QVQLVQSGAEVKKPGASVKVSCKASGGTFSNSIINWVRQAPGQGLEWMG
WMNPNSGNTNYAQKFQGRVTMTRDTSTSTVYMELSSLRSEDTAVYYCAR
EQWPSYWYFDLWGRGTLVTVSS,
(SEQ ID NO: 275)
QVQLVQSGAEVKKPGASVKVSCKASGGTFSTHDINWVRQAPGQGLEWMG
VINPSGGSAIYAQKFQGRVTMTRDTSTSTVYMELSSLRSEDTAVYYCAR
DRGYSYGYFDYWGQGTLVTVSS,
(SEQ ID NO: 276)
QVQLVQSGAEVKKPGASVKVSCKASGNTFIGYYVHWVRQAPGQGLEWVG
IINPNGGSISYAQKFQGRVTMTRDTSTSTVYMELSSLRSEDTAVYYCAR
GSGDPNYYYYYGLDVWGQGTTVTVSS,
(SEQ ID NO: 277)
QVQLVQSGAEVKKPGASVKVSCKASGYTLSYYYMHWVRQAPGQGLEWMG
MIGPSDGSTSYAQRFQGRVTMTRDTSTGTVYMELSSLRSEDTAVYYCAR
DTGDHFDYWGQGTLVTVSS,
(SEQ ID NO: 278)
QVQLVQSGAEVKKPGASVKVSCKASGYTFTGYYMHWVRQAPGQGLEWMG
IIGPSDGSTTYAQKFQGRVTMTRDTSTSTVYMELSSLRSEDTAVYYCAR
AENGMDVWGQGTTVTVSS,
(SEQ ID NO: 279)
QVQLVQSGAEVKKPGASVKVSCKASGYTFTGYYVHWVRQAPGQGLEWMG
IIAPSDGSTNYAQKFQGRVTMTRDTSTSTVYMELSSLRSEDTAVYYCAR
DPGGYMDVWGKGTTVTVSS,
(SEQ ID NO: 280)
QVQLVQSGAEVKKPGASVKVSCKASGYTFTGYYLHWVRQAPGQGLEWMG
MIGPSDGSTSYAQKFQGRVTMTRDTSTSTVYMELSSLRSEDTAVYYCAR
DGDAFDIWGQGTMVTVSS,
(SEQ ID NO: 281)
QVQLVQSGAEVKKPGSSVKVSCKASGYTFTGYYMHWVRQAPGQGLEWMG
RISPSDGSTTYAPKFQGRVTITADESTSTAYMELSSLRSEDTAVYYCAR
DMGDAFDIWGQGTTVTVSS,
(SEQ ID NO: 282)
QVQLVQSGAEVKKPGASVKVSCKASGYTFTGYYMHWVRQAPGQGLEWMG
MIGPSDGSTSYAQRFQGRVTMTRDTSTSTVYMELSSLRSEDTAVYYCAR
EEDGMDVWGQGTTVTVSS,
(SEQ ID NO: 277)
QVQLVQSGAEVKKPGASVKVSCKASGYTLSYYYMHWVRQAPGQGLEWMG
MIGPSDGSTSYAQRFQGRVTMTRDTSTGTVYMELSSLRSEDTAVYYCAR
DTGDHFDYWGQGTLVTVSS,
(SEQ ID NO: 283)
QVQLVQSGAEVKKPGSSVKVSCKASGGTFNNFAISWVRQAPGQGLEWMG
GIIPIFDATNYAQKFQGRVTFTADESTSTAYMELSSLRSEDTAVYYCAR
GEYSSGFFFVGWFDLWGRGTQVTVSS,
and
(SEQ ID NO: 284)
QVQLVQSGAEVKKPGASVKVSCKASGYNFTGYYMHWVRQAPGQGLEWMG
IIAPSDGSTNYAQKFQGRVTMTRDTSTSTVYMELSSLRSEDTAVYYCAR
ETGDDAFDIWGQGTMVTVSS.

VL

The ABP specific for A*01:01_ASSLPTTMNY (SEQ ID NO: 7) may comprise a VL sequence. The VL sequence may be selected from

(SEQ ID NO: 285)
DIQMTQSPSSLSASVGDRVTITCRASQGISNYLAWYQQKPGKAPKLLIY
AASSLQGGVPSRFSGSGSGTDFTLTISSLQPEDFATYYCQQYFTTPYTF
GQGTKLEIK,
(SEQ ID NO: 286)
DIQMTQSPSSLSASVGDRVTITCRASQSISRWLAWYQQKPGKAPKLLIF
DASRLQSGVPSRFSGSGSGTDFTLTISSLQPEDFATYYCQQAEAFPYTF
GQGTKVEIK,
(SEQ ID NO: 287)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPITF
GQGTRLEIK,
(SEQ ID NO: 288)
DIQMTQSPSSLSASVGDRVTITCRASQSISNYLNWYQQKPGKAPKLLIY
KASSLESGVPSRFSGSGSGTDFTLTISSLQPEDFATYYCQQSYIIPYTF
GQGTKLEIK,
(SEQ ID NO: 289)
DIQMTQSPSSLSASVGDRVTITCRASQSISNYLNWYQQKPGKAPKLLIY
AASSLQSGVPSRFSGSGSGTDFTLTISSLQPEDFATYYCHQTYSTPLTF
GQGTKVEIK,
(SEQ ID NO: 290)
DIQMTQSPSSLSASVGDRVTITCRASQGISNYLAWYQQKPGKAPKLLIY
SASNLQSGVPSRFSGSGSGTDFTLTISSLQPEDFATYYCQQAYSFPWTF
GQGTKVEIK,
(SEQ ID NO: 291)
DIQMTQSPSSLSASVGDRVTITCRASQNISSYLNWYQQKPGKAPKLLIY
AASSLQSGVPSRFSGSGSGTDFTLTISSLQPEDFATYYCQQGYSTPLTF
GQGTRLEIK,
(SEQ ID NO: 292)
DIQMTQSPSSLSASVGDRVTITCRASQDISRYLAWYQQKPGKAPKLLIY
DASNLETGVPSRFSGSGSGTDFTLTISSLQPEDFATYYCQQANSFPRTF
GQGTKVEIK,
(SEQ ID NO: 293)
DIQMTQSPSSLSASVGDRVTITCQASQDISNYLNWYQQKPGKAPKLLIY
AASNLQSGVPSRFSGSGSGTDFTLTISSLQPEDFATYYCQQANSLPYTF
GQGTKVEIK,
(SEQ ID NO: 294)
DIQMTQSPSSLSASVGDRVTITCRASQSISSYLNWYQQKPGKAPKLLIY
AASTLQNGVPSRFSGSGSGTDFTLTISSLQPEDFATYYCQQSYSTPFTF
GPGTKVDIK,
(SEQ ID NO: 295)
DIQMTQSPSSLSASVGDRVTITCRASQRISSYLNWYQQKPGKAPKLLIY
SASTLQSGVPSRFSGSGSGTDFTLTISSLQPEDFATYYCQQSYSTPFTF
GPGTKVDIK,
(SEQ ID NO: 296)
DIQMTQSPSSLSASVGDRVTITCRASQSISSYLAWYQQKPGKAPKLLIY
DASKLETGVPSRFSGSGSGTDFTLTISSLQPEDFATYYCQQSYGVPTFG
QGTKLEIK,
(SEQ ID NO: 297)
DIQMTQSPSSLSASVGDRVTITCRASQGISSWLAWYQQKPGKAPKLLIY
DASNLETGVPSRFSGSGSGTDFTLTISSLQPEDFATYYCQQSYSTPLTF
GGGTKVEIK,
(SEQ ID NO: 176)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIK,
(SEQ ID NO: 298)
DIQMTQSPSSLSASVGDRVTITCRASQGISTYLAWYQQKPGKAPKLLIY
DASSLQSGVPSRFSGSGSGTDFTLTISSLQPEDFATYYCQQYYSYPWTF
GQGTRLEIK,
(SEQ ID NO: 294)
DIQMTQSPSSLSASVGDRVTITCRASQSISSYLNWYQQKPGKAPKLLIY
AASTLQNGVPSRFSGSGSGTDFTLTISSLQPEDFATYYCQQSYSTPFTF
GPGTKVDIK,
(SEQ ID NO: 299)
DIVMTQSPLSLPVTPGEPASISCRSSQSLLHSNGYNYLDWYLQKPGQSP
QLLIYLGSNRASGVPDRFSGSGSGTDFTLKISRVEAEDVGVYYCMQTLK
TPLSFGGGTKVEIK,
and
(SEQ ID NO: 176)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIK.

VH-VL Combinations

The ABP specific for A*01:01_ASSLPTTMNY (SEQ ID NO: 7) may comprise a particular VH sequence and a particular VL sequence. In some embodiments, the ABP specific for A*01:01_ASSLPTTMNY (SEQ ID NO: 7) comprises a VH sequence and VL sequence from the scFv designated G10(1A07), G10(1B07), G10(1E12), G10(1F06), G10(1H01), G10(1H08), G10(2C04), G10(2G11), G10(3E04), G10(4A02), G10(4C05), G10(4D04), G10(4D10), G10(4E07), G10(4E12), G10(4G06), G10(5A08), or G10(5C08). The VH and VL sequences of identified scFvs that specifically bind A*01:01_ASSLPTTMNY (SEQ ID NO: 7) are shown in Table 8. For clarity, each identified scFv hit is designated a clone name, and each row contains the VH and VL sequences for that particular clone name. For example, the scFv identified by clone name G10(1A07) comprises the VH sequence

(SEQ ID NO: 268)
EVQLLESGGGLVKPGGSLRLSCAASGFTFSSYWMSWVRQAPGKGLEWVS
GISARSGRTYYADSVKGRFTISRDDSKNTLYLQMNSLKTEDTAVYYCAR
DQDTIFGVVITWFDPWGQGTLVTVSS
and
the VL sequence
(SEQ ID NO: 285)
DIQMTQSPSSLSASVGDRVTITCRASQGISNYLAWYQQKPGKAPKLLIY
AASSLQGGVPSRFSGSGSGTDFTLTISSLQPEDFATYYCQQYFTTPYTF
GQGTKLEIK.

Antibodies Specific for a*02:01_LLASSILCA (SEQ ID NO: 8) (G7)

In some aspects, provided herein are ABPs comprising antibodies or antigen-binding fragments thereof that specifically bind an HLA-PEPTIDE target, wherein the HLA Class I molecule of the HLA-PEPTIDE target is HLA subtype A*02:01 and the HLA-restricted peptide of the HLA-PEPTIDE target comprises, consists of, or consists essentially of the sequence LLASSILCA (SEQ ID NO: 8) (“G7”).

HLA-PEPTIDE target A*02:01_LLASSILCA (SEQ ID NO: 8), also referred to herein as “G7”, refers to an HLA-PEPTIDE target comprising the HLA-restricted peptide LLASSILCA (SEQ ID NO: 8) complexed with the HLA Class I molecule A*02:01, wherein the HLA-restricted peptide is located in the peptide binding groove of an α1/α2 heterodimer portion of the HLA Class I molecule. The restricted peptide is from tumor-specific gene product KKLC-1. HLA-PEPTIDE target A*02:01_LLASSILCA (SEQ ID NO: 8) is included in Table A2 as Target #6427.

Sequences of G7-Specific Antibodies

The ABP specific for A*02:01_LLASSILCA (SEQ ID NO: 8) may comprise one or more sequences, as described in further detail.

CDRs

The ABP specific for A*02:01_LLASSILCA (SEQ ID NO: 8) may comprise one or more antibody complementarity determining region (CDR) sequences, e.g., may comprise three heavy chain CDRs (CDR-H1, CDR-H2, CDR-H3) and three light chain CDRs (CDR-L1, CDR-L2, CDR-L3).

The ABP specific for A*02:01_LLASSILCA (SEQ ID NO: 8) may comprise a CDR-H3 sequence. The CDR-H3 sequence may be selected from

(SEQ ID NO: 300)
CARDGYDFWSGYTSDDYW,
(SEQ ID NO: 301)
CASDYGDYR,
(SEQ ID NO: 302)
CARDLMTTVVTPGDYGMDVW,
(SEQ ID NO: 303)
CARQDGGAFAFDIW,
(SEQ ID NO: 304)
CARELGYYYGMDVW,
(SEQ ID NO: 305)
CARALIFGVPLLPYGMDVW,
(SEQ ID NO: 306)
CAKDLATVGEPYYYYGMDVW,
and
(SEQ ID NO: 307)
CARLWFGELHYYYYYGMDVW.

The ABP specific for A*02:01_LLASSILCA (SEQ ID NO: 8) may comprise a CDR-L3 sequence. The CDR-L3 sequence may be selected from CHHYGRSHTF (SEQ ID NO: 308), CQQANAFPPTF (SEQ ID NO: 309), CQQYYSIPLTF (SEQ ID NO: 310), CQQSYSTPPTF (SEQ ID NO: 311), CQQSYSFPYTF (SEQ ID NO: 312), CMQALQTPLTF (SEQ ID NO: 93), CQQGNTFPLTF (SEQ ID NO: 313), and CMQGSHWPPSF (SEQ ID NO: 314).

The ABP specific for A*02:01_LLASSILCA (SEQ ID NO: 8) may comprise a particular heavy chain CDR3 (CDR-H3) sequence and a particular light chain CDR3 (CDR-L3) sequence. In some embodiments, the ABP comprises the CDR-H3 and the CDR-L3 from the scFv designated G7(1C06), G7(1G10), G7(1B04), G7(2C02), G7(1A03), G7(2E09), G7(1F08), or G7(3A09). CDR sequences of identified scFvs that specifically bind A*02:01_LLASSILCA (SEQ ID NO: 8) are shown in Table 30. For clarity, each identified scFv hit is designated a clone name, and each row contains the CDR sequences for that particular clone name. For example, the scFv identified by clone name G7(1C06) comprises the heavy chain CDR3 sequence CARDGYDFWSGYTSDDYW (SEQ ID NO: 300) and the light chain CDR3 sequence CHHYGRSHTF (SEQ ID NO: 308).

The ABP specific for A*02:01_LLASSILCA (SEQ ID NO: 8) may comprise all six CDRs from the scFv designated G7(1C06), G7(1G10), G7(1B04), G7(2C02), G7(1A03), G7(2E09), G7(1F08), or G7(3A09).

VL

The ABP specific for *02:01_LLASSILCA (SEQ ID NO: 8) may comprise a VL sequence. The VL sequence may be selected from

(SEQ ID NO: 315)
EIVMTQSPATLSVSPGERATLSCRASQSVSSSNLAWYQQKPGQAPRLLI
YGASTRATGIPARFSGSGSGTEFTLTISSLQSEDFAVYYCHHYGRSHTF
GQGTKVEIK,
(SEQ ID NO: 316)
DIQMTQSPSSLSASVGDRVTITCRASQDIRNDLGWYQQKPGKAPKLLIY
AASSLQSGVPSRFSGSGSGTDFTLTISSLQPEDFATYYCQQANAFPPTF
GQGTKVEIK,
(SEQ ID NO: 317)
DIVMTQSPDSLAVSLGERATINCKSSQSVFYSSNNKNQLAWYQQKPGQP
PKLLIYWASTRESGVPDRFSGSGSGTDFTLTISSLQAEDVAVYYCQQYY
SIPLTFGQGTKLEIK,
(SEQ ID NO: 318)
DIQMTQSPSSLSASVGDRVTITCQASQDIFKYLNWYQQKPGKAPKLLIY
AASTLQSGVPSRFSGSGSGTDFTLTISSLQPEDFATYYCQQSYSTPPTF
GQGTRLEIK,
(SEQ ID NO: 319)
DIQMTQSPSSLSASVGDRVTITCRASQSISTWLAWYQQKPGKAPKLLIY
YASSLQSGVPSRFSGSGSGTDFTLTISSLQPEDFATYYCQQSYSFPYTF
GQGTKVEIK,
(SEQ ID NO: 320)
DIVMTQSPLSLPVTPGEPASISCSSSQSLLHSNGYNYLDWYLQKPGQSP
QLLIYLGSNRASGVPDRFSGSGSGTDFTLKISRVEAEDVGVYYCMQALQ
TPLTFGGGTKVEIK,
(SEQ ID NO: 321)
DIQMTQSPSSLSASVGDRVTITCQASQDISNYLNWYQQKPGKAPKLLIY
SASNLRSGVPSRFSGSGSGTDFTLTISSLQPEDFATYYCQQGNTFPLTF
GQGTKVEIK,
and
(SEQ ID NO: 322)
DIVMTQSPLSLPVTPGEPASISCRSSQSLLHSNGYNYLDWYLQKPGQSP
QLLIYLGSNRASGVPDRFSGSGSGTDFTLKISRVEAEDVGVYYCMQGSH
WPPSFGQGTRLEIK.

VH

The ABP specific for *02:01_LLASSILCA (SEQ ID NO: 8) may comprise a VH sequence. The VH sequence may be selected from

(SEQ ID NO: 323)
QVQLVQSGAEVKKPGASVKVSCKASGGTFSNYGISWVRQAPGQGLEWMG
IINPGGSTSYAQKFQGRVTMTRDTSTSTVYMELSSLRSEDTAVYYCARD
GYDFWSGYTSDDYWGQGTLVTVSS,
(SEQ ID NO: 324)
EVQLLESGGGLVQPGGSLRLSCAASGFTFSSYAMHWVRQAPGKGLEWVS
GISGSGGSTYYADSVKGRFTISRDNSKNTLYLQMNSLRAEDTAVYYCAS
DYGDYRGQGTLVTVSS,
(SEQ ID NO: 325)
QVQLVQSGAEVKKPGASVKVSCKASGYTFSNYYIHWVRQAPGQGLEWMG
WLNPNSGNTGYAQRFQGRVTMTRDTSTSTVYMELSSLRSEDTAVYYCAR
DLMTTVVTPGDYGMDVWGQGTTVTVSS,
(SEQ ID NO: 326)
QVQLVQSGAEVKKPGASMKVSCKASGYTFTTDGISWVRQAPGQGLEWMG
RIYPHSGYTEYAKKFKGRVTMTRDTSTSTVYMELSSLRSEDTAVYYCAR
QDGGAFAFDIWGQGTMVTVSS,
(SEQ ID NO: 327)
QVQLVQSGAEVKKPGASVKVSCKASGYTFTSQYMHWVRQAPGQGLEWMG
WISPNNGDTNYAQKFQGRVTMTRDTSTSTVYMELSSLRSEDTAVYYCAR
ELGYYYGMDVWGQGTTVTVSS,
(SEQ ID NO: 328)
QVQLVQSGAEVKKPGSSVKVSCKASRYTFTSYDINWVRQAPGQGLEWMG
RIIPMLNIANYAPKFQGRVTITADESTSTAYMELSSLRSEDTAVYYCAR
ALIFGVPLLPYGMDVWGQGTTVTVSS,
(SEQ ID NO: 329)
EVQLLQSGGGLVQPGGSLRLSCAASGFTFSSSWMHWVRQAPGKGLEWVS
FISTSSGYIYYADSVKGRFTISRDNSKNTLYLQMNSLRAEDTAVYYCAK
DLATVGEPYYYYGMDVWGQGTTVTVSS,
and
(SEQ ID NO: 330)
QVQLVQSGAEVKKPGSSVKVSCKASGDTFNTYALSWVRQAPGQGLEWMG
WMNPNSGNAGYAQKFQGRVTITADESTSTAYMELSSLRSEDTAVYYCAR
LWFGELHYYYYYGMDVWGQGTMVTVSS.

VH-VL Combinations

The ABP specific for A*02:01_LLASSILCA (SEQ ID NO: 8) may comprise a particular VH sequence and a particular VL sequence. In some embodiments, the ABP specific for A*02:01_LLASSILCA (SEQ ID NO: 8) comprises a VH sequence and a VL sequence from the scFv designated G7(1C06), G7(1G10), G7(1B04), G7(2C02), G7(1A03), G7(2E09), G7(1F08), or G7(3A09). The VH and VL sequences of identified scFvs that specifically bind A*02:01_LLASSILCA (SEQ ID NO: 8) are shown in Table 29. For clarity, each identified scFv hit is designated a clone name, and each row contains the VH and VL sequences for that particular clone name. For example, the scFv identified by clone name G7(1C06) comprises the VH sequence

(SEQ ID NO: 323)
QVQLVQSGAEVKKPGASVKVSCKASGGTFSNYGISWVRQAPGQGLEWMG
IINPGGSTSYAQKFQGRVTMTRDTSTSTVYMELSSLRSEDTAVYYCARD
GYDFWSGYTSDDYWGQGTLVTVSS
and
the VL sequence
(SEQ ID NO: 315)
EIVMTQSPATLSVSPGERATLSCRASQSVSSSNLAWYQQKPGQAPRLLI
YGASTRATGIPARFSGSGSGTEFTLTISSLQSEDFAVYYCHHYGRSHTF
GQGTKVEIK.

Antibodies Specific for A*01:01_NTDNNLAVY (SEQ ID NO: 5) (G2)

In some aspects, provided herein are ABPs comprising antibodies or antigen-binding fragments thereof that specifically bind an HLA-PEPTIDE target, wherein the HLA Class I molecule of the HLA-PEPTIDE target is HLA subtype A*01:01 and the HLA-restricted peptide of the HLA-PEPTIDE target comprises, consists of, or consists essentially of the sequence NTDNNLAVY (SEQ ID NO: 5) (“G2”).

HLA-PEPTIDE target A*01:01_NTDNNLAVY (SEQ ID NO: 5), also referred to herein as “G2”, refers to an HLA-PEPTIDE target comprising the HLA-restricted peptide NTDNNLAVY (SEQ ID NO: 5) complexed with the HLA Class I molecule A*01:01, wherein the HLA-restricted peptide is located in the peptide binding groove of an α1/α2 heterodimer portion of the HLA Class I molecule. The restricted peptide is from tumor-specific gene product KKLC-1. HLA-PEPTIDE target A*01:01_NTDNNLAVY (SEQ ID NO: 5) is included in Table A1 as Target #33 and in Table A2 as Target #6500.

Sequences of G2-Specific Antibodies

The ABP specific for A*01:01_NTDNNLAVY (SEQ ID NO: 5) may comprise one or more sequences, as described in further detail.

CDRs

The ABP specific for A*01:01_NTDNNLAVY (SEQ ID NO: 5) may comprise one or more antibody complementarity determining region (CDR) sequences, e.g., may comprise three heavy chain CDRs (CDR-H1, CDR-H2, CDR-H3) and three light chain CDRs (CDR-L1, CDR-L2, CDR-L3).

The ABP specific for A*01:01_NTDNNLAVY (SEQ ID NO: 5) may comprise a CDR-H3 sequence. The CDR-H3 sequence may be selected from CAATEWLGVW (SEQ ID NO: 12), CARANWLDYW (SEQ ID NO: 13), CARANWLDYW (SEQ ID NO: 13), CARDWVLDYW (SEQ ID NO: 14), CARGEWLDYW (SEQ ID NO: 15), CARGWELGYW (SEQ ID NO: 16), CARDFVGYDDW (SEQ ID NO: 17), CARDYGDLDYW (SEQ ID NO: 18), CARGSYGMDVW (SEQ ID NO: 19), CARDGYSGLDVW (SEQ ID NO: 20), CARDSGVGMDVW (SEQ ID NO: 21), CARDGVAVASDYW (SEQ ID NO: 22), CARGVNVDDFDYW (SEQ ID NO: 23), CARGDYTGNWYFDLW (SEQ ID NO: 24), CARANWLDYW (SEQ ID NO: 13), CARDQFYGGNSGGHDYW (SEQ ID NO: 25), CAREEDYW (SEQ ID NO: 26), CARGDWFDPW (SEQ ID NO: 27), CARGDWFDPW (SEQ ID NO: 27), CARGEWFDPW (SEQ ID NO: 28), CARSDWFDPW (SEQ ID NO: 29), CARDSGSYFDYW (SEQ ID NO: 30), CARDYGGYVDYW (SEQ ID NO: 31), CAREGPAALDVW (SEQ ID NO: 32), CARERRSGMDVW (SEQ ID NO: 33), CARVLQEGMDVW (SEQ ID NO: 34), CASERELPFDIW (SEQ ID NO: 35), CAKGGGGYGMDVW (SEQ ID NO: 36), CAAMGIAVAGGMDVW (SEQ ID NO: 37), CARNWNLDYW (SEQ ID NO: 38), CATYDDGMDVW (SEQ ID NO: 39), CARGGGGALDYW (SEQ ID NO: 40), CALSGNYYGMDVW (SEQ ID NO: 41), CARGNPWELRLDYW (SEQ ID NO: 42), and CARDKNYYGMDVW (SEQ ID NO: 43).

The ABP specific for A*01:01_NTDNNLAVY (SEQ ID NO: 5) may comprise a CDR-L3 sequence. The CDR-L3 sequence may be selected from CQQSYNTPYTF (SEQ ID NO: 44), CQQSYSTPYTF (SEQ ID NO: 45), CQQSYSTPYSF (SEQ ID NO: 46), CQQSYSTPFTF (SEQ ID NO: 47), CQQSYGVPYTF (SEQ ID NO: 48), CQQSYSAPYTF (SEQ ID NO: 49), CQQSYSAPYTF (SEQ ID NO: 49), CQQSYSAPYSF (SEQ ID NO: 50), CQQSYSTPYTF (SEQ ID NO: 45), CQQSYSVPYSF (SEQ ID NO: 51), CQQSYSAPYTF (SEQ ID NO: 49), CQQSYSVPYSF (SEQ ID NO: 51), CQQSYSTPQTF (SEQ ID NO: 52), CQQLDSYPFTF (SEQ ID NO: 53), CQQSYSSPYTF (SEQ ID NO: 54), CQQSYSTPLTF (SEQ ID NO: 55), CQQSYSTPYSF (SEQ ID NO: 46), CQQSYSTPYTF (SEQ ID NO: 45), CQQSYSTPYTF (SEQ ID NO: 45), CQQSYSTPFTF (SEQ ID NO: 47), CQQSYSTPTF (SEQ ID NO: 56), CQQTYAIPLTF (SEQ ID NO: 57), CQQSYSTPYTF (SEQ ID NO: 45), CQQSYIAPFTF (SEQ ID NO: 58), CQQSYSIPLTF (SEQ ID NO: 59), CQQSYSNPTF (SEQ ID NO: 60), CQQSYSTPYSF (SEQ ID NO: 46), CQQSYSDQWTF (SEQ ID NO: 61), CQQSYLPPYSF (SEQ ID NO: 62), CQQSYSSPYTF (SEQ ID NO: 54), CQQSYTTPWTF (SEQ ID NO: 63), CQQSYLPPYSF (SEQ ID NO: 62), CQEGITYTF (SEQ ID NO: 64), CQQYYSYPFTF (SEQ ID NO: 65), and CQHYGYSPVTF (SEQ ID NO: 66).

The ABP specific for A*01:01_NTDNNLAVY (SEQ ID NO: 5) may comprise a particular heavy chain CDR3 (CDR-H3) sequence and a particular light chain CDR3 (CDR-L3) sequence. In some embodiments, the ABP comprises the CDR-H3 and the CDR-L3 from the scFv designated G2(2E07), G2(2E03), G2(2A11), G2(2C06), G2(1G01), G2(1C02), G2(1H01), G2(1B12), G2(1B06), G2(2H10), G2(1H10), G2(2C11), G2(1C09), G2(1A10), G2(1B10), G2(1D07), G2(1E05), G2(1D03), G2(1G12), G2(2H11), G2(1C03), G2(1G07), G2(1F12), G2(1G03), G2(2B08), G2(2A10), G2(2D04), G2(1C06), G2(2A09), G2(1B08), G2(1E03), G2(2A03), G2(2F01), G2(1H11), or G2(1D06). CDR sequences of identified scFvs that specifically bind A*01:01_NTDNNLAVY (SEQ ID NO: 5) are found in Table 28. For clarity, each identified scFv hit is designated a clone name, and each row contains the CDR sequences for that particular clone name. For example, the scFv identified by clone name G2(2E07) comprises the heavy chain CDR3 sequence CAATEWLGVW (SEQ ID NO: 12) and the light chain CDR3 sequence CQQSYNTPYTF (SEQ ID NO: 44).

The ABP specific for A*01:01_NTDNNLAVY (SEQ ID NO: 5) may comprise all six CDRs from the scFv designated G2(2E07), G2(2E03), G2(2A11), G2(2C06), G2(1G01), G2(1C02), G2(1H01), G2(1B12), G2(1B06), G2(2H10), G2(1H10), G2(2C11), G2(1C09), G2(1A10), G2(1B10), G2(1D07), G2(1E05), G2(1D03), G2(1G12), G2(2H11), G2(1C03), G2(1G07), G2(1F12), G2(1G03), G2(2B08), G2(2A10), G2(2D04), G2(1C06), G2(2A09), G2(1B08), G2(1E03), G2(2A03), G2(2F01), G2(1H11), or G2(1D06).

VL

The ABP specific for A*01:01_NTDNNLAVY (SEQ ID NO: 5) may comprise a VL sequence. The VL sequence may be selected from

(SEQ ID NO: 331)
DIQMTQSPSSLSASVGDRVTITCRASQSISTWLAWYQQKPGKAPKLLIYAASSLRSGV
PSRFSGSGSGTDFTLTISSLQPEDFATYYCQQSYNTPYTFGQGTKLEIK,
(SEQ ID NO: 332)
DIQMTQSPSSLSASVGDRVTITCRASQSISRWLAWYQQKPGKAPKLLIYAASTVQSG
VPSRFSGSGSGTDFTLTISSLQPEDFATYYCQQSYSTPYTFGQGTKLEIK,
(SEQ ID NO: 333)
DIQMTQSPSSLSASVGDRVTITCRASQDISRWLAWYQQKPGKAPKLLIYAASRLQAG
VPSRFSGSGSGTDFTLTISSLQPEDFATYYCQQSYSTPYSFGQGTKLEIK,
(SEQ ID NO: 334)
DIQMTQSPSSLSASVGDRVTITCRASQTISSWLAWYQQKPGKAPKLLIYAASSLQSGV
PSRFSGSGSGTDFTLTISSLQPEDFATYYCQQSYSTPFTFGPGTKVDIK,
(SEQ ID NO: 335)
DIQMTQSPSSLSASVGDRVTITCRASQTISSWLAWYQQKPGKAPKLLIYAASSLQSGV
PSRFSGSGSGTDFTLTISSLQPEDFATYYCQQSYGVPYTFGQGTKVEIK,
(SEQ ID NO: 336)
DIQMTQSPSSLSASVGDRVTITCRASQSISNWLAWYQQKPGKAPKLLIYAASSLQSGV
PSRFSGSGSGTDFTLTISSLQPEDFATYYCQQSYSAPYTFGPGTKVDIK,
(SEQ ID NO: 337)
DIQMTQSPSSLSASVGDRVTITCRASQSVGNWLAWYQQKPGKAPKLLIYGASSLQTG
VPSRFSGSGSGTDFTLTISSLQPEDFATYYCQQSYSAPYTFGQGTKVEIK,
(SEQ ID NO: 338)
DIQMTQSPSSLSASVGDRVTITCRASQNIGNWLAWYQQKPGKAPKLLIYAASTLQTG
VPSRFSGSGSGTDFTLTISSLQPEDFATYYCQQSYSAPYSFGQGTKLEIK,
(SEQ ID NO: 339)
DIQMTQSPSSLSASVGDRVTITCRASQSISRWLAWYQQKPGKAPKLLIYAASSLQSGV
PSRFSGSGSGTDFTLTISSLQPEDFATYYCQQSYSTPYTFGQGTKLEIK,
(SEQ ID NO: 340)
DIQMTQSPSSLSASVGDRVTITCRASQSISSWLAWYQQKPGKAPKLLIYGASSLQSGV
PSRFSGSGSGTDFTLTISSLQPEDFATYYCQQSYSVPYSFGQGTKLEIK,
(SEQ ID NO: 341)
DIQMTQSPSSLSASVGDRVTITCRASQSISKWLAWYQQKPGKAPKLLIYAASSLQSGV
PSRFSGSGSGTDFTLTISSLQPEDFATYYCQQSYSAPYTFGQGTKVEIK,
(SEQ ID NO: 342)
DIQMTQSPSSLSASVGDRVTITCRASQGISNYLAWYQQKPGKAPKLLIYAASTLQSGV
PSRFSGSGSGTDFTLTISSLQPEDFATYYCQQSYSVPYSFGQGTKLEIK,
(SEQ ID NO: 343)
DIQMTQSPSSLSASVGDRVTITCRASQTISNYLNWYQQKPGKAPKLLIYAASNLQSGV
PSRFSGSGSGTDFTLTISSLQPEDFATYYCQQSYSTPQTFGQGTKVEIK
(SEQ ID NO: 344)
DIQMTQSPSSLSASVGDRVTITCRASRDIGRAVGWYQQKPGKAPKLLIYAASSLQSG
VPSRFSGSGSGTDFTLTISSLQPEDFATYYCQQLDSYPFTFGPGTKVDIK,
(SEQ ID NO: 345)
DIQMTQSPSSLSASVGDRVTITCRASQSISSWLAWYQQKPGKAPKLLIYAASTLQSGV
PSRFSGSGSGTDFTLTISSLQPEDFATYYCQQSYSSPYTFGPGTKVDIK,
(SEQ ID NO: 346)
DIQMTQSPSSLSASVGDRVTITCQASQDISNYLNWYQQKPGKAPKLLIYAASSLQSGV
PSRFSGSGSGTDFTLTISSLQPEDFATYYCQQSYSTPLTFGGGTKLEIK
(SEQ ID NO: 347)
DIQMTQSPSSLSASVGDRVTITCRASQSIGRWLAWYQQKPGKAPKLLIYAASSLQSG
VPSRFSGSGSGTDFTLTISSLQPEDFATYYCQQSYSTPYSFGQGTKVEIK,
(SEQ ID NO: 348)
DIQMTQSPSSLSASVGDRVTITCRASQSISTWLAWYQQKPGKAPKLLIYAASTLQSGV
PSRFSGSGSGTDFTLTISSLQPEDFATYYCQQSYSTPYTFAQGTKLEIK,
(SEQ ID NO: 349)
DIQMTQSPSSLSASVGDRVTITCQASQDISNYLNWYQQKPGKAPKLLIYGASRLQSG
VPSRFSGSGSGTDFTLTISSLQPEDFATYYCQQSYSTPYTFGQGTKLEIK,
(SEQ ID NO: 350)
DIQMTQSPSSLSASVGDRVTITCRASQSISSWLAWYQQKPGKAPKLLIYAASTLQSGV
PSRFSGSGSGTDFTLTISSLQPEDFATYYCQQSYSTPFTFGPGTKVDIK,
(SEQ ID NO: 351)
DIQMTQSPSSLSASVGDRVTITCRASQSVSNWLAWYQQKPGKAPKLLIYAASSLQSG
VPSRFSGSGSGTDFTLTISSLQPEDFATYYCQQSYSTPTFGQGTKLEIK,
(SEQ ID NO: 352)
DIQMTQSPSSLSASVGDRVTITCQASQDISNYLNWYQQKPGKAPKLLIYAASTLQSG
VPSRFSGSGSGTDFTLTISSLQPEDFATYYCQQTYAIPLTFGGGTKVEIK,
(SEQ ID NO: 353)
DIQMTQSPSSLSASVGDRVTITCQASQDIGSWLAWYQQKPGKAPKLLIYATSSLQSG
VPSRFSGSGSGTDFTLTISSLQPEDFATYYCQQSYSTPYTFGQGTKLEIK,
(SEQ ID NO: 354)
DIQMTQSPSSLSASVGDRVTITCRASQGISRWLAWYQQKPGKAPKLLIYAASTLQPG
VPSRFSGSGSGTDFTLTISSLQPEDFATYYCQQSYIAPFTFGPGTKVDIK,
(SEQ ID NO: 355)
DIQMTQSPSSLSASVGDRVTITCRASQGISNYLAWYQQKPGKAPKLLIYAASRLESGV
PSRFSGSGSGTDFTLTISSLQPEDFATYYCQQSYSIPLTFGGGTKVEIK,
(SEQ ID NO: 356)
DIQMTQSPSSLSASVGDRVTITCRASQSISSYLNWYQQKPGKAPKLLIYGVSSLQSGV
PSRFSGSGSGTDFTLTISSLQPEDFATYYCQQSYSNPTFGQGTKVEIK,
(SEQ ID NO: 357)
DIQMTQSPSSLSASVGDRVTITCRASQSISSWVAWYQQKPGKAPKLLIYGASNLESGV
PSRFSGSGSGTDFTLTISSLQPEDFATYYCQQSYSTPYSFGQGTKLEIK,
(SEQ ID NO: 358)
DIQMTQSPSSLSASVGDRVTITCRASQGISNYLAWYQQKPGKAPKLLIYAASSLQSGV
PSRFSGSGSGTDFTLTISSLQPEDFATYYCQQSYSDQWTFGQGTKVEIK,
(SEQ ID NO: 359)
DIQMTQSPSSLSASVGDRVTITCRASQSISRWLAWYQQKPGKAPKLLIYAASSLQSGV
PSRFSGSGSGTDFTLTISSLQPEDFATYYCQQSYLPPYSFGQGTKVEIK,
(SEQ ID NO: 360)
DIQMTQSPSSLSASVGDRVTITCRASQSISNWLAWYQQKPGKAPKLLIYAASSLQSGV
PSRFSGSGSGTYFTLTISSLQPEDFATYYCQQSYSSPYTFGQGTKLEIK,
(SEQ ID NO: 361)
DIQMTQSPSSLSASVGDRVTITCRASQSISHYLNWYQQKPGKAPKLLIYGASSLQSGV
PSRFSGSGSGTDFTLTISSLQPEDFATYYCQQSYTTPWTFGQGTRLEIK,
(SEQ ID NO: 362)
DIQMTQSPSSLSASVGDRVTITCRASQSISSWLAWYQQKPGKAPKLLIYAASTLQSGV
PSRFSGSGSGTDFTLTISSLQPEDFATYYCQQSYLPPYSFGQGTKLEIK,
(SEQ ID NO: 363)
DIQMTQSPSSLSASVGDRVTITCQASQDISNYLNWYQQKPGKAPKLLIYGASRLQSG
VPSRFSGSGSGTDFTLTISSLQPEDFATYYCQEGITYTFGQGTKVEIK,
(SEQ ID NO: 364)
DIQMTQSPSSLSASVGDRVTITCQASQDISNYLNWYQQKPGKAPKLLIYAASSLQSGV
PSRFSGSGSGTDFTLTISSLQPEDFATYYCQQYYSYPFTFGPGTKVDIK,
and
(SEQ ID NO: 365)
EIVMTQSPATLSVSPGERATLSCRASQSVSRNLAWYQQKPGQAPRLLIYGASTRATGI
PARFSGSGSGTEFTLTISSLQSEDFAVYYCQHYGYSPVTFGQGTKLEIK.

VH

The ABP specific for A*01:01_NTDNNLAVY (SEQ ID NO: 5) may comprise a VH sequence. The VH sequence may be selected from

(SEQ ID NO: 366)
QVQLVQSGAEVKKPGASVKVSCKASGGTFSSATISWVRQAPGQGLEWMG
WIYPNSGGTVYAQKFQGRVTMTRDTSTSTVYMELSSLRSEDTAVYYCAA
TEWLGVWGQGTTVTVSS,
(SEQ ID NO: 367)
EVQLLQSGAEVKKPGSSVKVSCKASGGTFSSYAISWVRQAPGQGLEWMG
WINPNSGGTISAPNFQGRVTMTRDTSTSTVYMELSSLRSEDTAVYYCAR
ANWLDYWGQGTLVTVSS,
(SEQ ID NO: 368)
EVQLLESGAEVKKPGASVKVSCKASGYTFTTYDLAWVRQAPGQGLEWMG
WINPNSGGTNYAQKFQGRVTMTRDTSTSTVYMELSSLRSEDTAVYYCAR
ANWLDYWGQGTLVTVSS,
(SEQ ID NO: 369)
QVQLVQSGAEVKKPGASVKVSCKSSGYSFDSYVVNWVRQAPGQGLEWMG
WINPNSGGTNYAQKFQGRVTMTRDTSTSTVYMELSSLRSEDTAVYYCAR
DWVLDYWGQGTLVTVSS,
(SEQ ID NO: 370)
QVQLVQSGAEVKKPGASVKVSCKASGYTFTSYGISWVRQAPGQGLEWMG
WMNPNSGGTNYAQKFQGRVTMTRDTSTSTVYMELSSLRSEDTAVYYCAR
GEWLDYWGQGTLVTVSS,
(SEQ ID NO: 371)
QVQLVQSGAEVKKPGASVKVSCKASGYTFTSYGISWVRQAPGQGLEWMG
WINPNSGGTNYAQKFQGRVTMTRDTSTSTVYMELSSLRSEDTAVYYCAR
GWELGYWGQGTLVTVSS,
(SEQ ID NO: 372)
QVQLVQSGAEVKKPGASVKVSCKASGYTFTRYTINWVRQAPGQGLEWMG
WINPNSGGTNYAQKFQGRVTMTRDTSTSTVYMELSSLRSEDTAVYYCAR
DFVGYDDWGQGTLVTVSS,
(SEQ ID NO: 373)
QVQLVQSGAEVKKPGASVKVSCKASGYTFTSYGITWVRQAPGQGLEWMG
WINPNSGGTNYAQKFQGRVTMTRDTSTSTVYMELSSLRSEDTAVYYCAR
DYGDLDYWGQGTLVTVSS,
(SEQ ID NO: 374)
QVQLVQSGAEVKKPGASVKVSCKASGGTFSNYILSWVRQAPGQGLEWMG
WINPDSGGTNYAQKFQGRVTMTRDTSTSTVYMELSSLRSEDTAVYYCAR
GSYGMDVWGQGTTVTVSS,
(SEQ ID NO: 375)
QVQLVQSGAEVKKPGASVKVSCKASGYSFTRYNMHWVRQAPGQGLEWMG
WINPNSGGTNYAQKFQGRVTMTRDTSTSTVYMELSSLRSEDTAVYYCAR
DGYSGLDVWGKGTTVTVSS,
(SEQ ID NO: 376)
QVQLVQSGAEVKKPGASVKVSCKASGGTFSSYAISWVRQAPGQGLEWMG
WINPNNGGTNYAQKFQGRVTMTRDTSTSTVYMELSSLRSEDTAVYYCAR
DSGVGMDVWGQGTTVTVSS,
(SEQ ID NO: 377)
QVQLVQSGAEVKKPGASVKVSCKASGGTFNNYAFSWVRQAPGQGLEWMG
WINPNSGGTNYAQKFQGRVTMTRDTSTSTVYMELSSLRSEDTAVYYCAR
DGVAVASDYWGQGTLVTVSS,
(SEQ ID NO: 378)
QVQLVQSGAEVKKPGASVKVSCKASGYTFSSYNMHWVRQAPGQGLEWMG
WINGNTGGTNYAQKFQGRVTMTRDTSTSTVYMELSSLRSEDTAVYYCAR
GVNVDDFDYWGQGTLVTVSS,
(SEQ ID NO: 379)
QVQLVQSGAEVKKPGASVKVSCKASGGTFSSYAFSWVRQAPGQGLEWMG
WINPDTGYTRYAQKFQGRVTMTRDTSTSTVYMELSSLRSEDTAVYYCAR
GDYTGNWYFDLWGRGTLVTVSS,
(SEQ ID NO: 380)
EVQLLESGAEVKKPGASVKVSCKASGYTFTSYGISWVRQAPGQGLEWMG
WINPYSGGTNYAQKLQGRVTMTRDTSTSTVYMELSSLRSEDTAVYYCAR
ANWLDYWGQGTLVTVSS,
(SEQ ID NO: 381)
QVQLVQSGAEVKKPGASVKVSCKASGYTFTSYGISWVRQAPGQGLEWMG
WISAYNGYTNYAQNLQGRVTMTRDTSTSTVYMELSSLRSEDTAVYYCAR
DQFYGGNSGGHDYWGQGTLVTVSS,
(SEQ ID NO: 382)
QVQLVQSGAEVKKPGASVKVSCKASGYTFTDYNMHWVRQAPGQGLEWMG
WMNPNSGGTNYAQKFQGRVTMTRDTSTSTVYMELSSLRSEDTAVYYCAR
E-EDYWGQGTLVTVSS,
(SEQ ID NO: 383)
QVQLVQSGAEVKKPGASVKVSCKASGYTFTRYTINWVRQAPGQGLEWMG
WINPNSGGANYAQKFQGRVTMTRDTSTSTVYMELSSLRSEDTAVYYCAR
GDWFDPWGQGTLVTVSS,
(SEQ ID NO: 384)
QVQLVQSGAEVKKPGASVKVSCKASGYTFTSYLMHWVRQAPGQGLEWMG
WISPNSGGTNYAQKFQGRVTMTRDTSTSTVYMELSSLRSEDTAVYYCAR
GDWFDPWGQGTLVTVSS,
(SEQ ID NO: 385)
QVQLVQSGAEVKKPGASVKVSCKASGYTFSDYYVHWVRQAPGQGLEWMG
WINPNSGGTNYAQKFQGRVTMTRDTSTSTVYMELSSLRSEDTAVYYCAR
GEWFDPWGQGTLVTVSS,
(SEQ ID NO: 386)
QVQLVQSGAEVKKPGASVKVSCKASGYTFTTYYMHWVRQAPGQGLEWMG
WINPNSGGTNYAQKFQGRVTMTRDTSTSTVYMELSSLRSEDTAVYYCAR
SDWFDPWGQGTLVTVSS,
(SEQ ID NO: 387)
QVQLVQSGAEVKKPGASVKVSCKASGGTFSNYAINWVRQAPGQGLEWMG
WISPYSGGTNYAQKFQGRVTMTRDTSTSTVYMELSSLRSEDTAVYYCAR
DSGSYFDYWGQGTLVTVSS,
(SEQ ID NO: 388)
QVQLVQSGAEVKKPGASVKVSCKASGYTFTDYYMHWVRQAPGQGLEWMG
WIYPNTGGTNYAQKFQGRVTMTRDTSTSTVYMELSSLRSEDTAVYYCAR
DYGGYVDYWGQGTLVTVSS,
(SEQ ID NO: 389)
EVQLLESGAEVKKPGASVKVSCKASGYTFTSYAMNWVRQAPGQGLEWMG
WMNPNSGGTKYAQKFQGRVTMTRDTSTSTVYMELSSLRSEDTAVYYCAR
EGPAALDVWGQGTLVTVSS,
(SEQ ID NO: 390)
QVQLVQSGAEVKKPGASVKVSCKASGYTLTSHLIHWVRQAPGQGLEWMG
WINPNSGGTNYAQKFQGRVTMTRDTSTSTVYMELSSLRSEDTAVYYCAR
ERRSGMDVWGQGTTVTVSS,
(SEQ ID NO: 391)
EVQLLESGAEVKKPGASVKVSCKASGYSFTDYIVHWVRQAPGQGLEWMG
WINPYSGGTKYAQKFQGRVTMTRDTSTSTVYMELSSLRSEDTAVYYCAR
VLQEGMDVWGQGTLVTVSS,
(SEQ ID NO: 392)
QVQLVQSGAEVKKPGASVKVSCKASGYTFSNFLINWVRQAPGQGLEWMG
WINPNSGGTNYAQKFQGRVTMTRDTSTSTVYMELSSLRSEDTAVYYCAS
ERELPFDIWGQGTMVTVSS,
(SEQ ID NO: 393)
QVQLVQSGAEVKKPGASVKVSCKASGYTFTDYQMFWVRQAPGQGLEWMG
WINPNSGGTNYAQKFQGRVTMTRDTSTSTVYMELSSLRSEDTAVYYCAK
GGGGYGMDVWGQGTTVTVSS,
(SEQ ID NO: 394)
QVQLVQSGAEVKKPGASVKVSCKASGGTFSSYAISWVRQAPGQGLEWMG
WINPNSGGTNYAQKFQGRVTMTRDTSTSTVYMELSSLRSEDTAVYYCAA
MGIAVAGGMDVWGQGTLVTVSS,
(SEQ ID NO: 395)
QVQLVQSGAEVKKPGASVKVSCKASGYTFTNYHMHWVRQAPGQGLEWMG
WIHPDSGGTSYAQKFQGRVTMTRDTSTSTVYMELSSLRSEDTAVYYCAR
NWNLDYWGQGTLVTVSS,
(SEQ ID NO: 396)
QVQLVQSGAEVKKPGASVKVSCKASGYTFTGYYMHWVRQAPGQGLEWMG
WMNPNSGNTGYAQKFQGRVTMTRDTSTSTVYMELSSLRSEDTAVYYCAT
YDDGMDVWGQGTTVTVSS,
(SEQ ID NO: 397)
QVQLVQSGAEVKKPGASVKVSCKASGYTFTSYTVNWVRQAPGQGLEWMG
WINPNSGGTKYAQNFQGRVTMTRDTSTSTVYMELSSLRSEDTAVYYCAR
GGGGALDYWGQGTLVTVSS,
(SEQ ID NO: 398)
QVQLVQSGAEVKKPGASVKVSCKASGYTFTSYYMHWVRQAPGQGLEWMG
MINPRDDTTDYARDFQGRVTMTRDTSTSTVYMELSSLRSEDTAVYYCAL
SGNYYGMDVWGQGTTVTVSS,
(SEQ ID NO: 399)
QVQLVQSGAEVKKPGASVKVSCKASGYTFTNYYMHWVRQAPGQGLEWMG
MINPSGGGTSYAQKFQGRVTMTRDTSTSTVYMELSSLRSEDTAVYYCAR
GNPWELRLDYWGQGTLVTVSS,
and
(SEQ ID NO: 400)
QVQLVQSGAEVKKPGSSVKVSCKASGYTFTSQYMHWVRQAPGQGLEWMG
RIIPLLGIVNYAQKFQGRVTITADESTSTAYMELSSLRSEDTAVYYCAR
DKNYYGMDVWGQGTTVTVSS.

VH-VL Combinations

The ABP specific for A*01:01_NTDNNLAVY (SEQ ID NO: 5) may comprise a particular VH sequence and a particular VL sequence. In some embodiments, the ABP specific for A*01:01 NTDNNLAVY (SEQ ID NO: 5) comprises the VH sequence and the VL sequence from the scFv designated G2(2E07), G2(2E03), G2(2A11), G2(2C06), G2(1G01), G2(1C02), G2(1H01), G2(1B12), G2(1B06), G2(2H10), G2(1H10), G2(2C11), G2(1C09), G2(1A10), G2(1B10), G2(1D07), G2(1E05), G2(1D03), G2(1G12), G2(2H11), G2(1C03), G2(1G07), G2(1F12), G2(1G03), G2(2B08), G2(2A10), G2(2D04), G2(1C06), G2(2A09), G2(1B08), G2(1E03), G2(2A03), G2(2F01), G2(1H11), or G2(1D06). VH and VL sequences of identified scFvs that specifically bind A*01:01_NTDNNLAVY (SEQ ID NO: 5) are found in Table 27. For clarity, each identified scFv hit is designated a clone name, and each row contains the CDR sequences for that particular clone name. For example, the scFv identified by clone name G2(2E07) comprises the VH sequence

(SEQ ID NO: 366)
QVQLVQSGAEVKKPGASVKVSCKASGGTFSSATISWVRQAPGQGLEWMG
WIYPNSGGTVYAQKFQGRVTMTRDTSTSTVYMELSSLRSEDTAVYYCAA
TEWLGVWGQGTTVTVSS
and
the VL sequence
(SEQ ID NO: 331)
DIQMTQSPSSLSASVGDRVTITCRASQSISTWLAWYQQKPGKAPKLLIY
AASSLRSGVPSRFSGSGSGTDFTLTISSLQPEDFATYYCQQSYNTPYTF
GQGTKLEIK.

Receptors

Among the provided ABPs, e.g., HLA-PEPTIDE ABPs, are receptors. The receptors can include antigen receptors and other chimeric receptors that specifically bind an HLA-PEPTIDE target disclosed herein. The receptor may be a chimeric antigen receptor (CAR).

Also provided are cells expressing the receptors and uses thereof in adoptive cell therapy, such as treatment of diseases and disorders associated with HLA-PEPTIDE expression, including cancer.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Exemplary of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, e.g., and in which the antigen-binding portion, e.g., scFv, is replaced by an antibody, e.g., as provided herein.

Among the chimeric receptors are chimeric antigen receptors (CARs). The chimeric receptors, such as CARs, generally include an extracellular antigen binding domain that includes, is, or is comprised within, one of the provided anti-HLA-PEPTIDE ABPs such as anti-HLA-PEPTIDE antibodies. Thus, the chimeric receptors, e.g., CARs, typically include in their extracellular portions one or more HLA-PEPTIDE-ABPs, such as one or more antigen-binding fragment, domain, or portion, or one or more antibody variable domains, and/or antibody molecules, such as those described herein. In some embodiments, the CAR includes a HLA-PEPTIDE-binding portion or portions of the ABP (e.g., antibody) molecule, such as a variable heavy (VH) chain region and/or variable light (VL) chain region of the antibody, e.g., an scFv antibody fragment.

CARs

In an aspect, the ABPs provided herein, e.g., ABPs that specifically bind HLA-PEPTIDE targets disclosed herein, include CARs.

In some embodiments, the CAR is a recombinant CAR.

The recombinant CAR may be a human CAR, comprising fully human sequences, e.g., natural human sequences.

In some embodiments, the recombinant receptor such as a CAR, such as the antibody portion thereof, further includes a spacer, which may be or include at least a portion of an immunoglobulin constant region or variant or modified version thereof, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the constant region or portion is of IgD.

The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the HLA-PEPTIDE-specific binding component (e.g., ABP) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

The receptor, e.g., the CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the HLA-PEPTIDE-binding ABP (e.g., antibody) is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16.

In some embodiments, upon ligation of the CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the receptor. In other embodiments, the receptor does not include a component for generating a costimulatory signal. In some aspects, an additional receptor is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In some embodiments, the activating domain is included within one receptor, whereas the costimulatory component is provided by another receptor recognizing another antigen. In some embodiments, the receptors include activating or stimulatory receptors, and costimulatory receptors, both expressed on the same cell (see WO2014/055668). In some aspects, the HLA-PEPTIDE-targeting receptor is the stimulatory or activating receptor; in other aspects, it is the costimulatory receptor. In some embodiments, the cells further include inhibitory receptors (e.g., iCARs, see Fedorov et al., Sci. Transl. Medicine, 5(215) (December, 2013), such as a receptor recognizing an antigen other than HLA-PEPTIDE, whereby an activating signal delivered through the HLA-PEPTIDE-targeting receptor is diminished or inhibited by binding of the inhibitory receptor to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

In some cases, CARs are referred to as first, second, and/or third generation CARs. In some aspects, a first generation CAR is one that solely provides a CD3-chain induced signal upon antigen binding; in some aspects, a second-generation CAR is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing an antibody or fragment described herein. In some aspects, the chimeric antigen receptor includes an extracellular portion containing an antibody or fragment described herein and an intracellular signaling domain. In some embodiments, an antibody or fragment includes an scFv or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1).

In some embodiments, the chimeric antigen receptor contains an intracellular domain of a T cell costimulatory molecule. In some aspects, the T cell costimulatory molecule is CD28 or 41BB.

In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or U.S. Pat. No. 8,911,993.

In some aspects, the spacer contains only a hinge region of an IgG, such as only a hinge of IgG4 or IgG1. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody or fragment thereof, such as any of the HLA-PEPTIDE antibodies, including single chain antibodies (sdAbs, e.g. containing only the VH region) and scFvs, described herein, a spacer such as any of the Ig-hinge containing spacers, a CD28 transmembrane domain, a CD28 intracellular signaling domain, and a CD3 zeta signaling domain. In some embodiments, the CAR includes an antibody or fragment, such as any of the HLA-PEPTIDE antibodies, including sdAbs and scFvs described herein, a spacer such as any of the Ig-hinge containing spacers, a CD28 transmembrane domain, a CD28 intracellular signaling domain, and a CD3 zeta signaling domain.

### Engineered Cells

Also provided are cells such as cells that contain an antigen receptor, e.g., that contains an extracellular domain including an anti-HLA-PEPTIDE ABP (e.g., a CAR), described herein. Also provided are populations of such cells, and compositions containing such cells. In some embodiments, compositions or populations are enriched for such cells, such as in which cells expressing the HLA-PEPTIDE ABP make up at least 1, 5, 10, 20, 30, 40, 50, 60, 70, 80, 90, 91, 92, 93, 94, 95, 96, 97, 98, 99, or more than 99 percent of the total cells in the composition or cells of a certain type such as T cells or CD8+ or CD4+ cells. In some embodiments, a composition comprises at least one cell containing an antigen receptor disclosed herein. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing an ABP comprising a receptor, e.g., a CAR. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

The cells may be genetically modified to reduce expression or knock out endogenous TCRs. Such modifications are described in Mol Ther Nucleic Acids. 2012 December; 1(12): e63; Blood. 2011 Aug. 11; 118(6):1495-503; Blood. 2012 Jun. 14; 119(24): 5697-5705; Torikai, Hiroki et al “HLA and TCR Knockout by Zinc Finger Nucleases: Toward “off-the-Shelf” Allogeneic T-Cell Therapy for CD19+ Malignancies.” Blood 116.21 (2010): 3766; Blood. 2018 Jan. 18; 131(3):311-322. doi: 10.1182/blood-2017-05-787598; and WO2016069283, which are incorporated by reference in their entirety.

The cells may be genetically modified to promote cytokine secretion. Such modifications are described in Hsu C, Hughes M S, Zheng Z, Bray R B, Rosenberg S A, Morgan R A. Primary human T lymphocytes engineered with a codon-optimized IL-15 gene resist cytokine withdrawal-induced apoptosis and persist long-term in the absence of exogenous cytokine. J Immunol. 2005; 175:7226-34; Quintarelli C, Vera J F, Savoldo B, Giordano Attianese G M, Pule M, Foster A E, Co-expression of cytokine and suicide genes to enhance the activity and safety of tumor-specific cytotoxic T lymphocytes. Blood. 2007; 110:2793-802; and Hsu C, Jones S A, Cohen C J, Zheng Z, Kerstann K, Zhou J, Cytokine-independent growth and clonal expansion of a primary human CD8+ T-cell clone following retroviral transduction with the IL-15 gene. Blood. 2007; 109:5168-77.

Mismatching of chemokine receptors on T cells and tumor-secreted chemokines has been shown to account for the suboptimal trafficking of T cells into the tumor microenvironment. To improve efficacy of therapy, the cells may be genetically modified to increase recognition of chemokines in tumor micro environment. Examples of such modifications are described in Moon et al., Expression of a functional CCR2 receptor enhances tumor localization and tumor eradication by retargeted human T cells expressing a mesothelin-specific chimeric antibody receptor. Clin Cancer Res. 2011; 17: 4719-4730; and Craddock et al., Enhanced tumor trafficking of GD2 chimeric antigen receptor T cells by expression of the chemokine receptor CCR2b. J Immunother. 2010; 33: 780-788.

The cells may be genetically modified to enhance expression of costimulatory/enhancing receptors, such as CD28 and 41BB.

Adverse effects of T cell therapy can include cytokine release syndrome and prolonged B-cell depletion. Introduction of a suicide/safety switch in the recipient cells may improve the safety profile of a cell-based therapy. Accordingly, the cells may be genetically modified to include a suicide/safety switch. The suicide/safety switch may be a gene that confers sensitivity to an agent, e.g., a drug, upon the cell in which the gene is expressed, and which causes the cell to die when the cell is contacted with or exposed to the agent. Exemplary suicide/safety switches are described in Protein Cell. 2017 August; 8(8): 573-589. The suicide/safety switch may be HSV-TK. The suicide/safety switch may be cytosine deaminase, purine nucleoside phosphorylase, or nitroreductase. The suicide/safety switch may be RapaCIDe™, described in U.S. Patent Application Pub. No. US20170166877A1. The suicide/safety switch system may be CD20/Rituximab, described in Haematologica. 2009 September; 94(9): 1316-1320. These references are incorporated by reference in their entirety.

The CAR may be introduced into the recipient cell as a split receptor which assembles only in the presence of a heterodimerizing small molecule. Such systems are described in Science. 2015 Oct. 16; 350(6258): aab4077, and in U.S. Pat. No. 9,587,020, which are hereby incorporated by reference in its entirety.

In some embodiments, the cells include one or more nucleic acids, e.g., a polynucleotide encoding a CAR disclosed herein, wherein the polynucleotide is introduced via genetic engineering, and thereby express recombinant or genetically engineered receptors, e.g., CARs, as disclosed herein. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The nucleic acids may include a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

A construct or vector may be used to introduce the CAR into the recipient cell. Exemplary constructs are described herein. Polynucleotides encoding the alpha and beta chains of the CAR may in a single construct or in separate constructs. The polynucleotides encoding the alpha and beta chains may be operably linked to a promoter, e.g., a heterologous promoter. The heterologous promoter may be a strong promoter, e.g., EF1alpha, CMV, PGK1, Ubc, beta actin, CAG promoter, and the like. The heterologous promoter may be a weak promoter. The heterologous promoter may be an inducible promoter. Exemplary inducible promoters include, but are not limited to TRE, NFAT, GAL4, LAC, and the like. Other exemplary inducible expression systems are described in U.S. Pat. Nos. 5,514,578; 6,245,531; 7,091,038 and European Patent No. 0517805, which are incorporated by reference in their entirety.

The construct for introducing the CAR into the recipient cell may also comprise a polynucleotide encoding a signal peptide (signal peptide element). The signal peptide may promote surface trafficking of the introduced CAR. Exemplary signal peptides include, but are not limited to CD8 signal peptide, immunoglobulin signal peptides, where specific examples include GM-CSF and IgG Kappa. Such signal peptides are described in Trends Biochem Sci. 2006 October; 31(10):563-71. Epub 2006 Aug. 21; and An, et al. “Construction of a New Anti-CD19 Chimeric Antigen Receptor and the Anti-Leukemia Function Study of the Transduced T Cells.” Oncotarget 7.9 (2016): 10638-10649. PMC. Web. 16 Aug. 2018; which are hereby incorporated by reference in its entirety.

In some cases, e.g., cases wherein a marker gene is included in the construct, the construct may comprise a ribosomal skip sequence. The ribosomal skip sequence may be a 2A peptide, e.g., a P2A or T2A peptide. Exemplary P2A and T2A peptides are described in Scientific Reports volume 7, Article number: 2193 (2017), hereby incorporated by reference in its entirety. In some cases, a FURIN/PACE cleavage site is introduced upstream of the 2A element. FURIN/PACE cleavage sites are described in, e.g., http://www.nuolan.net/substrates.html. The cleavage peptide may also be a factor Xa cleavage site. In cases where the alpha and beta chains are expressed from a single construct or open reading frame, the construct may comprise an internal ribosome entry site (IRES).

The construct may further comprise one or more marker genes. Exemplary marker genes include but are not limited to GFP, luciferase, HA, lacZ. The marker may be a selectable marker, such as an antibiotic resistance marker, a heavy metal resistance marker, or a biocide resistant marker, as is known to those of skill in the art. The marker may be a complementation marker for use in an auxotrophic host. Exemplary complementation markers and auxotrophic hosts are described in Gene. 2001 Jan. 24; 263(1-2):159-69. Such markers may be expressed via an IRES, a frameshift sequence, a 2A peptide linker, a fusion with the CAR, or expressed separately from a separate promoter.

Exemplary vectors or systems for introducing receptors, e.g., CARs into recipient cells include, but are not limited to Adeno-associated virus, Adenovirus, Adenovirus+Modified vaccinia, Ankara virus (MVA), Adenovirus+Retrovirus, Adenovirus+Sendai virus, Adenovirus+Vaccinia virus, Alphavirus (VEE) Replicon Vaccine, Antisense oligonucleotide, Bifidobacterium longum, CRISPR-Cas9, E. coli, Flavivirus, Gene gun, Herpesviruses, Herpes simplex virus, Lactococcus lactis, Electroporation, Lentivirus, Lipofection, Listeria monocytogenes, Measles virus, Modified Vaccinia Ankara virus (MVA), mRNA Electroporation, Naked/Plasmid DNA, Naked/Plasmid DNA+Adenovirus, Naked/Plasmid DNA+Modified Vaccinia Ankara virus (MVA), Naked/Plasmid DNA+RNA transfer, Naked/Plasmid DNA+Vaccinia virus, Naked/Plasmid DNA+Vesicular stomatitis virus, Newcastle disease virus, Non-viral, PiggyBac™ (PB) Transposon, nanoparticle-based systems, Poliovirus, Poxvirus, Poxvirus+Vaccinia virus, Retrovirus, RNA transfer, RNA transfer+Naked/Plasmid DNA, RNA virus, Saccharomyces cerevisiae, Salmonella typhimurium, Semliki forest virus, Sendai virus, Shigella dysenteriae, Simian virus, siRNA, Sleeping Beauty transposon, Streptococcus mutans, Vaccinia virus, Venezuelan equine encephalitis virus replicon, Vesicular stomatitis virus, and Vibrio cholera.

In certain embodiments, the CAR is introduced into the recipient cell via adeno associated virus (AAV), adenovirus, CRISPR-CAS9, herpesvirus, lentivirus, lipofection, mRNA electroporation, PiggyBac™ (PB) Transposon, retrovirus, RNA transfer, or Sleeping Beauty transposon.

In some embodiments, a vector for introducing a CAR into a recipient cell is a viral vector. Exemplary viral vectors include adenoviral vectors, adeno-associated viral (AAV) vectors, lentiviral vectors, herpes viral vectors, retroviral vectors, and the like. Such vectors are described herein.

Nucleotides, Vectors, Host Cells, and Related Methods

Also provided are isolated nucleic acids encoding HLA-PEPTIDE ABPs, vectors comprising the nucleic acids, and host cells comprising the vectors and nucleic acids, as well as recombinant techniques for the production of the ABPs.

The nucleic acids may be recombinant. The recombinant nucleic acids may be constructed outside living cells by joining natural or synthetic nucleic acid segments to nucleic acid molecules that can replicate in a living cell, or replication products thereof. For purposes herein, the replication can be in vitro replication or in vivo replication.

For recombinant production of an ABP, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Exemplary vectors or constructs suitable for expressing an ABP, e.g., a CAR, antibody, or antigen binding fragment thereof, include, e.g., the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as AGTlO, AGTl1, AZapII (Stratagene), AEMBL4, and ANMl 149, are also suitable for expressing an ABP disclosed herein.

Illustrative examples of suitable host cells are provided below. These host cells are not meant to be limiting, and any suitable host cell may be used to produce the ABPs provided herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. lichenformis), Pseudomonas (P. aeruginosa), and Streptomyces. One useful E. coli cloning host is E. coli 294, although other strains such as E. coli B, E. coli X1776, and E. coli W3110 are also suitable.

In addition to prokaryotes, eukaryotic microbes such as filamentous fungi or yeast are also suitable cloning or expression hosts for HLA-PEPTIDE ABP-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the HLA-PEPTIDE ABP may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469; or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the ABP can be produced intracellularly, in the periplasmic space, or directly secreted into the medium. If the ABP is produced intracellularly, as a first step, the particulate debris, either host cells or lysed fragments, is removed, for example, by centrifugation or ultrafiltration. For example, Carter et al. (Bio/Technology, 1992, 10:163-167, incorporated by reference in its entirety) describes a procedure for isolating ABPs which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the ABP is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the ABP may be useful, for example, where the ABP accumulates in a cell as an insoluble aggregate, or where yields from periplasmic expression are low.

Where the ABP is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The ABP composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the ABP. Protein A can be used to purify ABPs that comprise human γ1, γ2, or γ4 heavy chains (Lindmark et al., J. Immunol. Meth., 1983, 62:1-13, incorporated by reference in its entirety). Protein G is useful for all mouse isotypes and for human 73 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the ABP comprises a CH3 domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the ABP of interest and contaminants may be subjected to low pH hydrophobic interaction chromatography using an elution buffer at a pH between about 2.5 to about 4.5, generally performed at low salt concentrations (e.g., from about 0 to about 0.25 M salt).

Methods of Making HLA-PEPTIDE ABPs

### HLA-PEPTIDE Antigen Preparation

The HLA-PEPTIDE antigen used for isolation or creation of the ABPs provided herein may be intact HLA-PEPTIDE or a fragment of HLA-PEPTIDE. The HLA-PEPTIDE antigen may be, for example, in the form of isolated protein or a protein expressed on the surface of a cell.

In some embodiments, the HLA-PEPTIDE antigen is a non-naturally occurring variant of HLA-PEPTIDE, such as a HLA-PEPTIDE protein having an amino acid sequence or post-translational modification that does not occur in nature.

In some embodiments, the HLA-PEPTIDE antigen is truncated by removal of, for example, intracellular or membrane-spanning sequences, or signal sequences. In some embodiments, the HLA-PEPTIDE antigen is fused at its C-terminus to a human IgG1 Fc domain or a polyhistidine tag.

### Methods of Identifying ABPs

ABPs that bind HLA-PEPTIDE can be identified using any method known in the art, e.g., phage display or immunization of a subject.

One method of identifying an antigen binding protein includes providing at least one HLA-PEPTIDE target; and binding the at least one target with an antigen binding protein, thereby identifying the antigen binding protein. The antigen binding protein can be present in a library comprising a plurality of distinct antigen binding proteins.

In some embodiments, the library is a phage display library. The phage display library can be developed so that it is substantially free of antigen binding proteins that non-specifically bind the HLA of the HLA-PEPTIDE target. The antigen binding protein can be present in a yeast display library comprising a plurality of distinct antigen binding proteins. The yeast display library can be developed so that it is substantially free of antigen binding proteins that non-specifically bind the HLA of the HLA-PEPTIDE target.

In some embodiments, the library is a yeast display library.

In some embodiments, the library is a TCR display library. Exemplary TCR display libraries and methods of using such TCR display libraries are described in WO 98/39482; WO 01/62908; WO 2004/044004; WO2005116646, WO2014018863, WO2015136072, WO2017046198; and Helmut et al, (2000) PNAS 97 (26) 14578-14583, which are hereby incorporated by reference in their entirety.

In some aspects, the binding step is performed more than once, optionally at least three times, e.g., at least 1, 2, 3, 4, 5, 6, 7, 8, 9, or 10×.

In addition, the method can also include contacting the antigen binding protein with one or more peptide-HLA complexes that are distinct from the HLA-PEPTIDE target to determine if the antigen binding protein selectively binds the HLA-PEPTIDE target.

Another method of identifying an antigen binding protein can include obtaining at least one HLA-PEPTIDE target; administering the HLA-PEPTIDE target to a subject (e.g., a mouse, rabbit or a llama), optionally in combination with an adjuvant; and isolating the antigen binding protein from the subject. Isolating the antigen binding protein can include screening the serum of the subject to identify the antigen binding protein. The method can also include contacting the antigen binding protein with one or more peptide-HLA complexes that are distinct from the HLA-PEPTIDE target, e.g., to determine if the antigen binding protein selectively binds to the HLA-PEPTIDE target. An antigen binding protein that is identified can be humanized.

In some aspects, isolating the antigen binding protein comprises isolating a B cell from the subject that expresses the antigen binding protein. The B cell can be used to create a hybridoma. The B cell can also be used for cloning one or more of its CDRs. The B cell can also be immortalized, for example, by using EBV transformation. Sequences encoding an antigen binding protein can be cloned from immortalized B cells or can be cloned directly from B cells isolated from an immunized subject. A library that comprises the antigen binding protein of the B cell can also be created, optionally wherein the library is phage display or yeast display.

Another method of identifying an antigen binding protein can include obtaining a cell comprising the antigen binding protein; contacting the cell with an HLA-multimer (e.g., a tetramer) comprising at least one HLA-PEPTIDE target; and identifying the antigen binding protein via binding between the HLA-multimer and the antigen binding protein.

The cell can be, e.g., a T cell, optionally a cytotoxic T lymphocyte (CTL), or a natural killer (NK) cell, for example. The method can further include isolating the cell, optionally using flow cytometry, magnetic separation, or single cell separation. The method can further include sequencing the antigen binding protein.

Another method of identifying an antigen binding protein can include obtaining one or more cells comprising the antigen binding protein; activating the one or more cells with at least one HLA-PEPTIDE target presented on at least one antigen presenting cell (APC); and identifying the antigen binding protein via selection of one or more cells activated by interaction with at least one HLA-PEPTIDE target.

The cell can be, e.g., a T cell, optionally a CTL, or an NK cell, for example. The method can further include isolating the cell, optionally using flow cytometry, magnetic separation, or single cell separation. The method can further include sequencing the antigen binding protein.

### Methods of Making Monoclonal ABPs

Monoclonal ABPs may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal ABPs may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing ABPs that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal ABPs: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of ABP by the selected ABP-producing cells, and are sensitive media conditions, such as the presence or absence of HAT medium. Among these, preferred myeloma cell lines are murine myeloma lines, such as those derived from MOPC-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal ABPs. See e.g., Kozbor, J. Immunol., 1984, 133:3001, incorporated by reference in its entirety.

After the identification of hybridoma cells that produce ABPs of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal ABPs may be readily isolated and sequenced using conventional procedures (e.g., by using oligonucleotide probes that are capable of binding specifically to genes encoding the heavy and light chains of the monoclonal ABPs). Thus, the hybridoma cells can serve as a useful source of DNA encoding ABPs with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce ABP, to produce the monoclonal ABPs.

### Methods of Making Chimeric ABPs

Illustrative methods of making chimeric ABPs are described, for example, in U.S. Pat. No. 4,816,567; and Morrison et al., Proc. Natl. Acad. Sci. USA, 1984, 81:6851-6855; each of which is incorporated by reference in its entirety. In some embodiments, a chimeric ABP is made by using recombinant techniques to combine a non-human variable region (e.g., a variable region derived from a mouse, rat, hamster, rabbit, or non-human primate, such as a monkey) with a human constant region.

### Methods of Making Humanized ABPs

Humanized ABPs may be generated by replacing most, or all, of the structural portions of a non-human monoclonal ABP with corresponding human ABP sequences. Consequently, a hybrid molecule is generated in which only the antigen-specific variable, or CDR, is composed of non-human sequence. Methods to obtain humanized ABPs include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

### Methods of Making Human ABPs

Human ABPs can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human ABPs can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human ABPs may also be generated by in vitro activated B cells (see e.g., U.S. Pat. Nos. 5,567,610 and 5,229,275, each of which is incorporated by reference in its entirety). Human ABPs may also be derived from yeast-based libraries (see e.g., U.S. Pat. No. 8,691,730, incorporated by reference in its entirety).

### Methods of Making ABP Fragments

The ABP fragments provided herein may be made by any suitable method, including the illustrative methods described herein or those known in the art. Suitable methods include recombinant techniques and proteolytic digestion of whole ABPs. Illustrative methods of making ABP fragments are described, for example, in Hudson et al., Nat. Med., 2003, 9:129-134, incorporated by reference in its entirety. Methods of making scFv ABPs are described, for example, in Plückthun, in The Pharmacology of Monoclonal ABPs, vol. 113, Rosenburg and Moore eds., Springer-Verlag, New York, pp. 269-315 (1994); WO 93/16185; and U.S. Pat. Nos. 5,571,894 and 5,587,458; each of which is incorporated by reference in its entirety.

### Methods of Making Alternative Scaffolds

The alternative scaffolds provided herein may be made by any suitable method, including the illustrative methods described herein or those known in the art. For example, methods of preparing Adnectins™ are described in Emanuel et al., mAbs, 2011, 3:38-48, incorporated by reference in its entirety. Methods of preparing iMabs are described in U.S. Pat. Pub. No. 2003/0215914, incorporated by reference in its entirety. Methods of preparing Anticalins® are described in Vogt and Skerra, Chem. Biochem., 2004, 5:191-199, incorporated by reference in its entirety. Methods of preparing Kunitz domains are described in Wagner et al., Biochem. & Biophys. Res. Comm., 1992, 186:118-1145, incorporated by reference in its entirety. Methods of preparing thioredoxin peptide aptamers are provided in Geyer and Brent, Meth. Enzymol., 2000, 328:171-208, incorporated by reference in its entirety. Methods of preparing Affibodies are provided in Fernandez, Curr. Opinion in Biotech., 2004, 15:364-373, incorporated by reference in its entirety. Methods of preparing DARPins are provided in Zahnd et al., J. Mol. Biol., 2007, 369:1015-1028, incorporated by reference in its entirety. Methods of preparing Affilins are provided in Ebersbach et al., J. Mol. Biol., 2007, 372:172-185, incorporated by reference in its entirety. Methods of preparing Tetranectins are provided in Graversen et al., J. Biol. Chem., 2000, 275:37390-37396, incorporated by reference in its entirety. Methods of preparing Avimers are provided in Silverman et al., Nature Biotech., 2005, 23:1556-1561, incorporated by reference in its entirety. Methods of preparing Fynomers are provided in Silacci et al., J. Biol. Chem., 2014, 289:14392-14398, incorporated by reference in its entirety. Further information on alternative scaffolds is provided in Binz et al., Nat. Biotechnol., 2005 23:1257-1268; and Skerra, Current Opin. in Biotech., 2007 18:295-304, each of which is incorporated by reference in its entirety.

### Methods of Making Multispecific ABPs

The multispecific ABPs provided herein may be made by any suitable method, including the illustrative methods described herein or those known in the art. Methods of making common light chain ABPs are described in Merchant et al., Nature Biotechnol., 1998, 16:677-681, incorporated by reference in its entirety. Methods of making tetravalent bispecific ABPs are described in Coloma and Morrison, Nature Biotechnol., 1997, 15:159-163, incorporated by reference in its entirety. Methods of making hybrid immunoglobulins are described in Milstein and Cuello, Nature, 1983, 305:537-540; and Staerz and Bevan, Proc. Nat. Acad. Sci. USA, 1986, 83:1453-1457; each of which is incorporated by reference in its entirety. Methods of making immunoglobulins with knobs-into-holes modification are described in U.S. Pat. No. 5,731,168, incorporated by reference in its entirety. Methods of making immunoglobulins with electrostatic modifications are provided in WO 2009/089004, incorporated by reference in its entirety. Methods of making bispecific single chain ABPs are described in Traunecker et al., EMBO J., 1991, 10:3655-3659; and Gruber et al., J. Immunol., 1994, 152:5368-5374; each of which is incorporated by reference in its entirety. Methods of making single-chain ABPs, whose linker length may be varied, are described in U.S. Pat. Nos. 4,946,778 and 5,132,405, each of which is incorporated by reference in its entirety. Methods of making diabodies are described in Hollinger et al., Proc. Natl. Acad. Sci. USA, 1993, 90:6444-6448, incorporated by reference in its entirety. Methods of making triabodies and tetrabodies are described in Todorovska et al., J. Immunol. Methods, 2001, 248:47-66, incorporated by reference in its entirety. Methods of making trispecific F(ab′)3 derivatives are described in Tutt et al. J. Immunol., 1991, 147:60-69, incorporated by reference in its entirety. Methods of making cross-linked ABPs are described in U.S. Pat. No. 4,676,980; Brennan et al., Science, 1985, 229:81-83; Staerz, et al. Nature, 1985, 314:628-631; and EP 0453082; each of which is incorporated by reference in its entirety. Methods of making antigen-binding domains assembled by leucine zippers are described in Kostelny et al., J. Immunol., 1992, 148:1547-1553, incorporated by reference in its entirety. Methods of making ABPs via the DNL approach are described in U.S. Pat. Nos. 7,521,056; 7,550,143; 7,534,866; and 7,527,787; each of which is incorporated by reference in its entirety. Methods of making hybrids of ABP and non-ABP molecules are described in WO 93/08829, incorporated by reference in its entirety, for examples of such ABPs. Methods of making DAF ABPs are described in U.S. Pat. Pub. No. 2008/0069820, incorporated by reference in its entirety. Methods of making ABPs via reduction and oxidation are described in Carlring et al., PLoS One, 2011, 6:e22533, incorporated by reference in its entirety. Methods of making DVD-Igs™ are described in U.S. Pat. No. 7,612,181, incorporated by reference in its entirety. Methods of making DARTs™ are described in Moore et al., Blood, 2011, 117:454-451, incorporated by reference in its entirety. Methods of making DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety. Methods of making ABPs comprising scFvs fused to the C-terminus of the CH3 from an IgG are described in Coloma and Morrison, Nature Biotechnol., 1997, 15:159-163, incorporated by reference in its entirety. Methods of making ABPs in which a Fab molecule is attached to the constant region of an immunoglobulin are described in Miler et al., J. Immunol., 2003, 170:4854-4861, incorporated by reference in its entirety. Methods of making CovX-Bodies are described in Doppalapudi et al., Proc. Natl. Acad. Sci. USA, 2010, 107:22611-22616, incorporated by reference in its entirety. Methods of making Fcab ABPs are described in Wozniak-Knopp et al., Protein Eng. Des. Sel., 2010, 23:289-297, incorporated by reference in its entirety. Methods of making TandAb® ABPs are described in Kipriyanov et al., J. Mol. Biol., 1999, 293:41-56 and Zhukovsky et al., Blood, 2013, 122:5116, each of which is incorporated by reference in its entirety. Methods of making tandem Fabs are described in WO 2015/103072, incorporated by reference in its entirety. Methods of making Zybodies™ are described in LaFleur et al., mAbs, 2013, 5:208-218, incorporated by reference in its entirety.

### Methods of Making Variants

Any suitable method can be used to introduce variability into a polynucleotide sequence(s) encoding an ABP, including error-prone PCR, chain shuffling, and oligonucleotide-directed mutagenesis such as trinucleotide-directed mutagenesis (TRIM). In some aspects, several CDR residues (e.g., 4-6 residues at a time) are randomized. CDR residues involved in antigen binding may be specifically identified, for example, using alanine scanning mutagenesis or modeling. CDR-H3 and CDR-L3 in particular are often targeted for mutation.

The introduction of diversity into the variable regions and/or CDRs can be used to produce a secondary library. The secondary library is then screened to identify ABP variants with improved affinity. Affinity maturation by constructing and reselecting from secondary libraries has been described, for example, in Hoogenboom et al., Methods in Molecular Biology, 2001, 178:1-37, incorporated by reference in its entirety.

Methods for Engineering Cells with ABPs

Also provided are methods, nucleic acids, compositions, and kits, for expressing the ABPs, including receptors comprising antibodies, and CARs, and for producing genetically engineered cells expressing such ABPs. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II. 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298; Van Tedeloo et al. (2000) Gene Therapy 7(16): 1431-1437; and Roth et al. (2018) Nature 559:405-409). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

Preparation of Engineered Cells

In some embodiments, preparation of the engineered cells includes one or more culture and/or preparation steps. The cells for introduction of the HLA-PEPTIDE-ABP, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using CD3/CD28 conjugated magnetic beads (e.g., DYNABEADS™. M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a peripheral blood mononuclear cell (PBMC) sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. Peripheral blood mononuclear cell (PBMC) can be enriched for or depleted of CD62L−CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or ROR1, and positive selection based on a marker characteristic of central memory T cells, such as CD62L or CCR7, where the positive and negative selections are carried out in either order.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immune-magnetic (or affinity-magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference in its entirety. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labeled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale fluorescence activated cell sorting (FACS). In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This can then be diluted 1:1 with media so that the final concentration of DMSO and HSA are 10% and 4%, respectively. Other examples include Cryostor®, CTL-Cryo™ ABC freezing media, and the like. The cells are then frozen to −80 degrees C. at a rate of 1 degree per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some embodiments, the PBMC feeder cells are inactivated with Mytomicin C. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Methods of Isolating ABPs Based on Avidity

In the manufacture of multi-chain molecules there are usually product related contaminants/by-products that need to be cleared. Methods to purify antibodies based on avidity are known in the art. For example, Lindhofer et al. made a bispecific antibody with a heterodimeric heavy chain having one heavy chain with high affinity for Protein A (mouse IgG) and one heavy chain of low affinity for Protein A (rat IgG) and demonstrated separation of the rat/mouse heterodimeric bispecific antibody from a quadroma mixture of mouse/mouse and rat/rat dimers. (See Lindhofer et al. J. Immunol., 1995, 155(1):219-225, which is incorporated herein in its entirety). U.S. Pat. No. 8,586,713 (incorporated herein in its entirety) described a bispecific antibody format providing ease of isolation, comprising immunoglobulin heavy chain variable domains that are differentially modified in the CH3 domain, and at least one of the modifications results in a differential affinity for the bispecific antibody for an affinity reagent such as Protein A. Others have described use of affinity chromatography to separate antibodies comprising one Kappa domain and one lambda domain. See, for example, Wang, Chunlei, et al. MAbs. 2018, 10(8), and U.S. application Ser. Nos. 16/488,746 and 16/601,121, each of which is incorporated herein by reference.

The present disclosure provides methods for purifying ABPs based on the number of light chain Kappa constant domains in ABPs relative to contaminants (e.g. other antibodies or antibody fragments). The purification methods are based on differences in avidity to anti-Kappa resin comprising a ligand that binds to the light chain Kappa constant domain. Provided herein are methods of purifying ABPs having Kappa constant domains, wherein the ABP to be purified has a different number of light chain Kappa constant domains than contaminants (e.g., other antibodies or antibody fragments). For example, when there are multiple ABP, antibodies, and/or antibody fragments within a mixture or solution and the ABP, antibodies, and/or antibody fragments differ in number of light chain Kappa constant domains, the present disclosure provides a method for isolating the ABP of interest based on avidity to an anti-Kappa resin. The ABP and contaminants are contacted with the anti-Kappa resin during affinity chromatography to allow for differential binding to the anti-Kappa resin and during elution there is differential detachment of the ABP relative to contaminants because of differences in avidity to the anti-Kappa resin.

Conventional antibody purification processes involve a capture step (e.g Protein A), aimed largely at removing process related impurities (e.g. Host-cell protein, Host-cell DNA etc.), followed by multiple polishing steps (e.g., hydrophobic interaction chromatography, mixed-mode chromatography, ion exchange chromatography, etc.), aimed at removing product related impurities such as aggregates, in addition to remaining process-related impurities. Purification of multispecific antibodies (e.g. multispecific assymetric antibodies) is that expression of such molecules results in complex mixtures that comprise of additional product related impurities (e.g. homodimer contaminants, and increased aggregate levels) in the feedstream. Employing conventional antibody purification processes for purification of multispecific asymmetric antibodies at commercial scale may be less efficient, especially if they have to necessarily include, for example, multiple affinity steps to eliminate homodimer contaminants individually, or, for example, have to include additional polishing steps than is usually required to specifically isolate the ABP from the complex mixtures. One benefit of the purification methods disclosed herein is that they allow for elimination of many hard to remove homodimer contaminants present in the complex mixtures of the ABPs in a single step early in the purification process, thus minimizing the burden on downstream polishing steps, and possibly enabling simpler purification processes for these molecules.

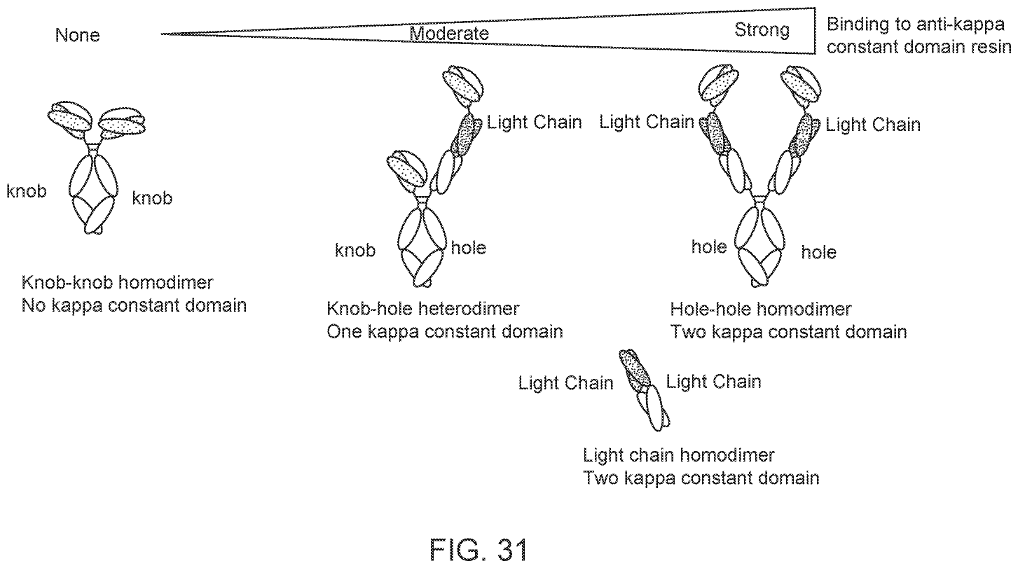

As shown in FIG. 31, antibodies having no Kappa constant domains, a single Kappa constant domain, and two Kappa constant domains differ in avidity to the anti-Kappa resin. In certain embodiments, the Format 4 antibodies are knob-hole heterodimers, having a single light chain Kappa constant domain. In some embodiments, the ABPs have lower avidity to the anti-Kappa resin than contaminants having a greater number of Kappa constant domains relative to the ABP. In some embodiments, the ABPs have higher avidity to the anti-Kappa resin having a fewer kappa constant domains relative to the ABP. In some embodiments, the methods described herein result in successful separation of this species from contaminants (e.g., antibody species having no Kappa constant domains (e.g. knob-knob homodimers) and antibody species having two Kappa constant domains (e.g. hole-hole homodimers and/or LC dimers)). The purification can be conducted using e.g. CaptureSelect KappaXP Chromatography, CaptureSelect KappaXL Chromatography, or KappaSelect Chromatography. CaptureSelect KappaXP, CaptureSelect Kappa XL and KappaSelect are affinity matrices that specifically bind to the constant domain of the Kappa light chain (LC).

In some embodiments, the anti-Kappa resin comprises a ligand that specifically binds to an epitope in a light chain Kappa constant domain. In some embodiments, the ligand is a monoclonal antibody. In some embodiments, the ligand is a camelid antibody. In some embodiments, the ligand does not bind to an epitope outside of the light chain Kappa constant domain. In some embodiments, the ligand does not bind to a Kappa variable domain.

In some embodiments, in the method of purification described herein, species (or contaminants) with no constant domains do not bind the resin, species with fewer constant domains elute first, and species with more constant domains elute later.

In some embodiments, the method comprises: (a) providing (i) a mixture that comprises an ABP comprising a light chain Kappa constant domain, optionally wherein the ABP is selected from any one of the preceding claims, and (ii) an anti-Kappa resin, wherein the anti-Kappa resin comprises a ligand having high specificity for a light chain Kappa constant domain, and wherein contaminants lacking a light chain Kappa constant domain do not bind the anti-Kappa resin; (b) contacting (i) and (ii) under conditions that allow for differential binding to the anti-Kappa resin as compared to a contaminant, in the mixture, that lacks a Kappa constant domain or has a different number of Kappa constant domains relative to the ABP; and (c) eluting the ABP from the anti-Kappa resin under conditions that allow for differential detachment of the ABP relative to the contaminant. In some embodiments, the contaminant is an antibody or antibody fragment. In some embodiments, the ABP of interest is a Format 4 antibody having only one Kappa constant domain. In some embodiments, the purification method is used to collect one antibody species, wherein the antibody species comprises a Kappa constant domain, and its contaminants, prior to purification have no light chain Kappa constant domains or a different number of light chain Kappa constant domains than the collected species (or collected contaminants).

Non-limiting examples of anti-Kappa resins that are contemplated in the present disclosure include: KappaSelect™ Affinity Matrix, CaptureSelect™ KappaXP Affinity Matrix, or CaptureSelect™ KappaXL Affinity Matrix. In certain embodiments, the anti-Kappa resin is CaptureSelect™ KappaXP Affinity Matrix.

The conditions that allow for differential detachment of the ABP and separate elution relative to the other contaminants in the mixture can include, without limitation, a pH gradient elution salt gradient elution, or step elution. In some embodiments, the method of purification described herein utilizes one or more of the following types of elution: salt gradient, salt step, pH gradient and pH step.

In some embodiments, an ionic modifier is used. Ionic modifiers can be selected from salts, beryllium, lithium, sodium, and potassium salts of acetate; sodium and potassium bicarbonates; lithium, sodium, potassium, and cesium carbonates; lithium, sodium, potassium, cesium, and magnesium chlorides; sodium and potassium fluorides; sodium, potassium, and calcium nitrates; sodium and potassium phosphates; and calcium and magnesium sulfates.

The use of either a continuous salt (ionic-strength) gradient or a pH gradient results in separation of species. In some embodiments, the salt is an inorganic salt. In some embodiments, the inorganic salt is sodium chloride (NaCl) or potassium chloride (KCl). In some embodiments, the inorganic salt is sodium chloride. In some embodiments, the salt gradient used for elution is a descending salt concentration gradient (from high salt concentration to low salt concentration). For example, during elution, the high (starting) salt concentration is 500-150 mM of NaCl and the low (final) salt concentration is 50-0 mM of NaCl. In some embodiments, the salt gradient is a gradient of about 500 mM of NaCl to about 50 mM of NaCl. In some embodiments, the salt gradient is a gradient of about 500 mM of NaCl to about 0 mM of NaCl. In some embodiments, the salt gradient is a gradient of about 200 mM of NaCl to about 50 mM of NaCl. In some embodiments, the salt gradient is a gradient of about 200 mM of NaCl to 0 mM of NaCl. In some embodiments, the salt gradient is a gradient of about 150 mM of NaCl to 0 mM of NaCl.

In some embodiments, the salt gradient is a gradient of about 140 mM of NaCl to about 0 mM of NaCl; about 160 mM of NaCl to about 0 mM of NaCl; about 180 mM of NaCl to about 0 mM of NaCl; about 200 mM of NaCl to about 0 mM of NaCl; about 220 mM of NaCl to about 0 mM of NaCl; about 240 mM of NaCl to about 0 mM of NaCl; about 260 mM of NaCl to about 0 mM of NaCl; about 280 mM of NaCl to about 0 mM of NaCl; about 300 mM of NaCl to about 0 mM of NaCl; about 320 mM of NaCl to about 0 mM of NaCl; about 340 mM of NaCl to about 0 mM of NaCl; about 360 mM of NaCl to about 0 mM of NaCl; about 380 mM of NaCl to about 0 mM of NaCl; about 400 mM of NaCl to about 0 mM of NaCl; about 420 mM of NaCl to about 0 mM of NaCl; about 440 mM of NaCl to about 0 mM of NaCl; about 460 mM of NaCl to about 0 mM of NaCl; about 480 mM of NaCl to about 0 mM of NaCl; about 500 mM of NaCl to about 0 mM of NaCl; about 520 mM of NaCl to about 0 mM of NaCl or about 540 mM of NaCl to about 0 mM of NaCl. In some embodiments, the salt gradient is a gradient of about 140 mM of NaCl to about 50 mM of NaCl; about 160 mM of NaCl to about 50 mM of NaCl; about 180 mM of NaCl to about 50 mM of NaCl; about 200 mM of NaCl to about 50 mM of NaCl; about 220 mM of NaCl to about 50 mM of NaCl; about 240 mM of NaCl to about 50 mM of NaCl; about 260 mM of NaCl to about 50 mM of NaCl; about 280 mM of NaCl to about 50 mM of NaCl; about 300 mM of NaCl to about 50 mM of NaCl; about 320 mM of NaCl to about 50 mM of NaCl; about 340 mM of NaCl to about 50 mM of NaCl; about 360 mM of NaCl to about 50 mM of NaCl; about 380 mM of NaCl to about 50 mM of NaCl; about 400 mM of NaCl to about 50 mM of NaCl; about 420 mM of NaCl to about 50 mM of NaCl; about 440 mM of NaCl to about 50 mM of NaCl; about 460 mM of NaCl to about 50 mM of NaCl; about 480 mM of NaCl to about 50 mM of NaCl; about 500 mM of NaCl to about 50 mM of NaCl; about 520 mM of NaCl to about 50 mM of NaCl; or about 540 mM of NaCl to about 50 mM of NaCl.

In some embodiments, the pH gradient elution is a gradient of about 6 to about 3. In some embodiments, the pH gradient elution is a gradient of about 5.5 to about 2.5, about 5.6 to about 2.5, about 5.7 to about 2.5, about 5.8 to about 2.5, about 5.9 to about 2.5, about 6 to about 2.5, about 6.1 to about 2.5, about 6.2 to about 2.5, about 6.3 to about 2.5, about 6.4 to about 2.5, or about 6.5 to about 2.5. In some embodiments, the pH gradient elution is a gradient of about 6.0 to about 3, about 5.9 to about 3, about 5.8 to about 3, about 5.7 to about 3, about 5.5 to about 3, about 5.6 to about 3, about 5.7 to about 3, about 5.8 to about 3, about 5.9 to about 3, about 6 to about 3, about 6.1 to about 3, about 6.2 to about 3, about 6.3 to about 3, about 6.4 to about 3, about 6.5 to about 3, about 5.5 to about 3.5, about 5.6 to about 3.5, about 5.7 to about 3.5, about 5.8 to about 3.5, about 5.9 to about 3.5, about 6 to about 3.5, about 6.1 to about 3.5, about 6.2 to about 3.5, about 6.3 to about 3.5, about 6.4 to about 3.5, or about 6.5 to about 3.5.

Step elution is also contemplated to isolate an ABP of interest using the methods described herein. Step elution has the advantage of scalability, particularly for large-scale manufacturing. It is a more efficient and economical approach to purification, allowing for high concentration and lower volume eluates.

In some embodiments, conditions that allow for differential detachment of the ABP and separate elution relative to the other contaminants in the mixture can comprise, without limitation, step variation in pH level and/or a step variation in salt concentration. In some embodiments, conditions that allow for differential detachment of the ABP and separate elution relative to the other contaminants in the mixture comprise step variation in pH level and/or a step variation in salt concentration.

In some embodiments, the ABP is eluted at the highest purity at 4.2, thus, the step variation in pH includes a pH of 4.2. In some embodiments, the step variation comprises a pH of about 3.5, about 3.6, about 3.7, about 3.8, about 3.9, about 4, about 4.1, about 4.2, about 4.3, about 4.4 or about 4.5. In some embodiments, the ABP is eluted at the highest purity at 3.9, thus, the step variation in pH includes a pH of 3.9. In some embodiments, step variation of salt concentration and step variation of pH are combined, and the step variation in pH includes a pH of 3.9. In some embodiments, the step variation comprises a pH of about 3.5, about 3.6, about 3.7, about 3.8, about 3.9, about 4, about 4.1, about 4.2, about 4.3, about 4.4 or about 4.5.

In some embodiments, the step variation comprises a pH selected from 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6.

In some embodiments, the step variation comprises a pH selected within the range of about 3 to about 5.0, about 3.1 to about 5.0, about 3.2 to about 5.0, about 3.3 to about 5.0, about 3.4 to about 5.0, about 3.5 to about 5.0, about 3.6 to about 5.0, about 3.7 to about 5.0, about 3.8 to about 5.0, about 3.9 to about 5.0, about 4 to about 5.0, about 4.1 to about 5.0, about 4.2 to about 5.0, about 4.3 to about 5.0, about 4.4 to about 5.0, about 4.5 to about 5.0, about 4.6 to about 5.0, about 4.7 to about 5.0, about 4.8 to about 5.0, about 4.9 to about 5.0, 3 to about 4.9, about 3.1 to about 4.9, about 3.2 to about 4.9, about 3.3 to about 4.9, about 3.4 to about 4.9, about 3.5 to about 4.9, about 3.6 to about 4.9, about 3.7 to about 4.9, about 3.8 to about 4.9, about 3.9 to about 4.9, about 4 to about 4.9, about 4.1 to about 4.9, about 4.2 to about 4.9, about 4.3 to about 4.9, about 4.4 to about 4.9, about 4.5 to about 4.9, about 4.6 to about 4.9, about 4.7 to about 4.9, about 4.8 to about 4.9, about 3 to about 4.8, about 3.1 to about 4.8, about 3.2 to about 4.8, about 3.3 to about 4.8, about 3.4 to about 4.8, about 3.5 to about 4.8, about 3.6 to about 4.8, about 3.7 to about 4.8, about 3.8 to about 4.8, about 3.9 to about 4.8, about 4 to about 4.8, about 4.1 to about 4.8, about 4.2 to about 4.8, about 4.3 to about 4.8, about 4.4 to about 4.8, about 4.5 to about 4.8, about 4.6 to about 4.8, about 4.7 to about 4.8, about 3 to about 4.7, about 3.1 to about 4.7, about 3.2 to about 4.7, about 3.3 to about 4.7, about 3.4 to about 4.7, about 3.5 to about 4.7, about 3.6 to about 4.7, about 3.7 to about 4.7, about 3.8 to about 4.7, about 3.9 to about 4.7, about 4 to about 4.7, about 4.1 to about 4.7, about 4.2 to about 4.7, about 4.3 to about 4.7, about 4.4 to about 4.7, about 4.5 to about 4.7, about 4.6 to about 4.7, about 3 to about 4.6, about 3.1 to about 4.6, about 3.2 to about 4.6, about 3.3 to about 4.6, about 3.4 to about 4.6, about 3.5 to about 4.6, about 3.6 to about 4.6, about 3.7 to about 4.6, about 3.8 to about 4.6, about 3.9 to about 4.6, about 4 to about 4.6, about 4.1 to about 4.6, about 4.2 to about 4.6, about 4.3 to about 4.6, about 4.4 to about 4.6, about 4.5 to about 4.6, about 3 to about 4.5, about 3.1 to about 4.5, about 3.2 to about 4.5, about 3.3 to about 4.5, about 3.4 to about 4.5, about 3.5 to about 4.5, about 3.6 to about 4.5, about 3.7 to about 4.5, about 3.8 to about 4.5, about 3.9 to about 4.5, about 4 to about 4.5, about 4.1 to about 4.5, about 4.2 to about 4.5, about 4.3 to about 4.5, about 4.4 to about 4.5, about 3 to about 4.4, about 3.1 to about 4.4, about 3.2 to about 4.4, about 3.3 to about 4.4, about 3.4 to about 4.4, about 3.5 to about 4.4, about 3.6 to about 4.4, about 3.7 to about 4.4, about 3.8 to about 4.4, about 3.9 to about 4.4, about 4 to about 4.4, about 4.1 to about 4.4, about 4.2 to about 4.4, or about 4.3 to about 4.4.

In some embodiments, the step variation in salt concentration utilizes an inorganic salt (e.g. NaCl). In some embodiments, the step variation in salt concentration comprises about 50 mM of NaCl, about 100 mM of NaCl, about 150 mM of NaCl, and/or 200 mM of NaCl. In some embodiments, the step variation in salt concentration comprises a salt concentration selected from a range of about 50 mM of NaCl to about 150 mM of NaCl. In some embodiments, the step variation in salt concentration comprises about 0 mM of NaCl, about 2 mM of NaCl, about 4 mM of NaCl, about 6 mM of NaCl, about 8 mM of NaCl, about 10 mM of NaCl, about 12 mM of NaCl, about 14 mM of NaCl, about 16 mM of NaCl, about 18 mM of NaCl, about 20 mM of NaCl, about 22 mM of NaCl, about 24 mM of NaCl, about 26 mM of NaCl, about 28 mM of NaCl, about 30 mM of NaCl, about 32 mM of NaCl, about 34 mM of NaCl, about 36 mM of NaCl, about 38 mM of NaCl, about 40 mM of NaCl, about 42 mM of NaCl, about 44 mM of NaCl, about 46 mM of NaCl, about 48 mM of NaCl, about 50 mM of NaCl, about 52 mM of NaCl, about 54 mM of NaCl, about 56 mM of NaCl, about 58 mM of NaCl, about 60 mM of NaCl, about 62 mM of NaCl, about 64 mM of NaCl, about 66 mM of NaCl, about 68 mM of NaCl, about 70 mM of NaCl, about 72 mM of NaCl, about 74 mM of NaCl, about 76 mM of NaCl, about 78 mM of NaCl, about 80 mM of NaCl, about 82 mM of NaCl, about 84 mM of NaCl, about 86 mM of NaCl, about 88 mM of NaCl, about 90 mM of NaCl, about 92 mM of NaCl, about 94 mM of NaCl, about 96 mM of NaCl, about 98 mM of NaCl, about 100 mM of NaCl, about 102 mM of NaCl, about 104 mM of NaCl, about 106 mM of NaCl, about 108 mM of NaCl, about 110 mM of NaCl, about 112 mM of NaCl, about 114 mM of NaCl, about 116 mM of NaCl, about 118 mM of NaCl, about 120 mM of NaCl, about 122 mM of NaCl, about 124 mM of NaCl, about 126 mM of NaCl, about 128 mM of NaCl, about 130 mM of NaCl, about 132 mM of NaCl, about 134 mM of NaCl, about 136 mM of NaCl, about 138 mM of NaCl, about 140 mM of NaCl, about 142 mM of NaCl, about 144 mM of NaCl, about 146 mM of NaCl, about 148 mM of NaCl, about 150 mM of NaCl, about 152 mM of NaCl, about 154 mM of NaCl, about 156 mM of NaCl, about 158 mM of NaCl, about 160 mM of NaCl, about 162 mM of NaCl, about 164 mM of NaCl, about 166 mM of NaCl, about 168 mM of NaCl, about 170 mM of NaCl, about 172 mM of NaCl, about 174 mM of NaCl, about 176 mM of NaCl, about 178 mM of NaCl, about 180 mM of NaCl, about 182 mM of NaCl, about 184 mM of NaCl, about 186 mM of NaCl, about 188 mM of NaCl, about 190 mM of NaCl, about 192 mM of NaCl, about 194 mM of NaCl, about 196 mM of NaCl, about 198 mM of NaCl, about 200 mM of NaCl, about 202 mM of NaCl, about 204 mM of NaCl, about 206 mM of NaCl, about 208 mM of NaCl, about 210 mM of NaCl, about 212 mM of NaCl, about 214 mM of NaCl, about 216 mM of NaCl, about 218 mM of NaCl, about 220 mM of NaCl, about 222 mM of NaCl, about 224 mM of NaCl, about 226 mM of NaCl, about 228 mM of NaCl, about 230 mM of NaCl, about 232 mM of NaCl, about 234 mM of NaCl, about 236 mM of NaCl, about 238 mM of NaCl, about 240 mM of NaCl, about 242 mM of NaCl, about 244 mM of NaCl, about 246 mM of NaCl, about 248 mM of NaCl, or about 250 mM of NaCl.

Exemplary methods for eluting these ABP are provided in the Examples section.

Assays

A variety of assays known in the art may be used to identify and characterize an HLA-PEPTIDE ABP provided herein.

Binding, Competition, and Epitope Mapping Assays

Specific antigen-binding activity of an ABP provided herein may be evaluated by any suitable method, including using SPR, BLI, RIA and MSD, as described elsewhere in this disclosure. Additionally, antigen-binding activity may be evaluated by ELISA assays, using flow cytometry, and/or Western blot assays.

Assays for measuring competition between two ABPs, or an ABP and another molecule (e.g., one or more ligands of HLA-PEPTIDE such as a TCR) are described elsewhere in this disclosure and, for example, in Harlow and Lane, ABPs: A Laboratory Manual ch. 14, 1988, Cold Spring Harbor Laboratory, Cold Spring Harbor, N.Y, incorporated by reference in its entirety.

Assays for mapping the epitopes to which an ABP provided herein bind are described, for example, in Morris “Epitope Mapping Protocols,” in Methods in Molecular Biology vol. 66, 1996, Humana Press, Totowa, N.J., incorporated by reference in its entirety. In some embodiments, the epitope is determined by peptide competition. In some embodiments, the epitope is determined by mass spectrometry. In some embodiments, the epitope is determined by mutagenesis. In some embodiments, the epitope is determined by crystallography.

### Assays for Effector Functions

Effector function following treatment with an ABP and/or cell provided herein may be evaluated using a variety of in vitro and in vivo assays known in the art, including those described in Ravetch and Kinet, Annu. Rev. Immunol., 1991, 9:457-492; U.S. Pat. Nos. 5,500,362, 5,821,337; Hellstrom et al., Proc. Nat'l Acad. Sci. USA, 1986, 83:7059-7063; Hellstrom et al., Proc. Nat'l Acad. Sci. USA, 1985, 82:1499-1502; Bruggemann et al., J. Exp. Med., 1987, 166:1351-1361; Clynes et al., Proc. Nat'l Acad. Sci. USA, 1998, 95:652-656; WO 2006/029879; WO 2005/100402; Gazzano-Santoro et al., J. Immunol. Methods, 1996, 202:163-171; Cragg et al., Blood, 2003, 101:1045-1052; Cragg et al. Blood, 2004, 103:2738-2743; and Petkova et al., Int'l. Immunol., 2006, 18:1759-1769; each of which is incorporated by reference in its entirety.

### Cytotoxicity Assays

Assays for evaluating cytotoxicity of the ABPs provided herein are described elsewhere in this disclosure.

### Size Exclusion Chromatography (SEC-HPLC)

Assays for separation and identification of ABPs or fragments thereof using SEC-HPLC are described elsewhere in this disclosure.

### Capillary Electrophoresis Sodium Dodecyl Sulfate (CE-SDS) Assays

Assays for separation and identification of ABPs or fragments thereof using CE-SDS are described elsewhere in this disclosure.

Pharmaceutical Compositions

An ABP, cell, or HLA-PEPTIDE target provided herein can be formulated in any appropriate pharmaceutical composition and administered by any suitable route of administration. Suitable routes of administration include, but are not limited to, the intra-arterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, propylene glycol, and combinations thereof.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, monosodium glutamate, and combinations thereof.

In some embodiments, the pharmaceutical composition comprises a carrier or filler. Illustrative examples of carriers or fillers include lactose, maltodextrin, mannitol, sorbitol, chitosan, stearic acid, xanthan gum, guar gum, and combinations thereof.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, vitamin E polyethylene(glycol) succinate, and combinations thereof.

In some embodiments, the pharmaceutical composition comprises an anti-caking agent. Illustrative examples of anti-caking agents include calcium phosphate (tribasic), hydroxymethyl cellulose, hydroxypropyl cellulose, magnesium oxide, and combinations thereof.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, sugars, and combinations thereof. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an ABP, since water can facilitate the degradation of some ABPs.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In certain embodiments, an ABP and/or cell provided herein is formulated as parenteral dosage forms. Parenteral dosage forms can be administered to subjects by various routes including, but not limited to, subcutaneous, intravenous (including infusions and bolus injections), intramuscular, and intra-arterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry (e.g., lyophilized) products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the ABPs and/or cells disclosed herein can also be incorporated into the parenteral dosage forms.

In some embodiments, the parenteral dosage form is lyophilized. Exemplary lyophilized formulations are described, for example, in U.S. Pat. Nos. 6,267,958 and 6,171,586; and WO 2006/044908; each of which is incorporated by reference in its entirety.

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic ABP.

The amount of the ABP, cell, or composition which will be effective in the prevention or treatment of a disorder or one or more symptoms thereof will vary with the nature and severity of the disease or condition, and the route by which the ABP and/or cell is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the ABPs and/or cells provided herein are also encompassed by the dosage amounts and dose frequency schedules provided herein. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an ABP or composition provided herein followed by one or more maintenance doses.

In certain embodiments, a dose of an ABP, cell, or composition provided herein can be administered to achieve a steady-state concentration of the ABP and/or cell in blood or serum of the subject. The steady-state concentration can be determined by measurement according to techniques available to those of skill or can be based on the physical characteristics of the subject such as height, weight and age.

As discussed in more detail elsewhere in this disclosure, an ABP and/or cell provided herein may optionally be administered with one or more additional agents useful to prevent or treat a disease or disorder. The effective amount of such additional agents may depend on the amount of ABP present in the formulation, the type of disorder or treatment, and the other factors known in the art or described herein.

Therapeutic Applications

For therapeutic applications, ABPs and/or cells are administered to a mammal, generally a human, in a pharmaceutically acceptable dosage form such as those known in the art and those discussed above. For example, ABPs and/or cells may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The ABPs also are suitably administered by peritumoral, intralesional, or perilesional routes, to exert local as well as systemic therapeutic effects. The intraperitoneal route may be particularly useful, for example, in the treatment of ovarian tumors.

The ABPs and/or cells provided herein can be useful for the treatment of any disease or condition involving HLA-PEPTIDE. In some embodiments, the disease or condition is a disease or condition that can benefit from treatment with an anti-HLA-PEPTIDE ABP and/or cell. In some embodiments, the disease or condition is a tumor. In some embodiments, the disease or condition is a cell proliferative disorder. In some embodiments, the disease or condition is a cancer. In some embodiments, the disease or condition is a viral infection (or viral disease), e.g. chronic viral disease.

In some embodiments, the ABPs and/or cells provided herein are provided for use as a medicament. In some embodiments, the ABPs and/or cells provided herein are provided for use in the manufacture or preparation of a medicament. In some embodiments, the medicament is for the treatment of a disease or condition that can benefit from an anti-HLA-PEPTIDE ABP and/or cell. In some embodiments, the disease or condition is a tumor. In some embodiments, the disease or condition is a cell proliferative disorder. In some embodiments, the disease or condition is a cancer.

In some embodiments, provided herein is a method of treating a disease or condition in a subject in need thereof by administering an effective amount of an ABP and/or cell provided herein to the subject. In some aspects, the disease or condition is a cancer.

In some embodiments, provided herein is a method of treating a disease or condition in a subject in need thereof by administering an effective amount of an ABP and/or cell provided herein to the subject, wherein the disease or condition is a cancer, and the cancer is selected from a solid tumor and a hematological tumor.

In some embodiments, provided herein is a method of modulating an immune response in a subject in need thereof, comprising administering to the subject an effective amount of an ABP and/or cell or a pharmaceutical composition disclosed herein.

In some embodiments, the ABPs are administered at an effective amount or therapeutically effective amount. As used herein, the term “therapeutically effective amount” or “effective amount” refers to an amount of an ABP or pharmaceutical composition provided herein that, when administered to a subject, is effective to treat a disease or disorder. An “effective amount” of a compound is at least the minimum amount required to achieve the desired therapeutic or prophylactic result, such as a measurable improvement or prevention of a particular disorder (e.g., cancer). As would be appreciated by one of ordinary skill in the art, an effective amount can vary according to factors such as the disease state, age, sex, and weight of the patient, and the ability of the antibody to elicit a desired response in the individual.

Combination Therapies

In some embodiments, an ABP and/or cell provided herein is administered with at least one additional therapeutic agent. Any suitable additional therapeutic agent may be administered with an ABP and/or cell provided herein. An additional therapeutic agent can be fused to an ABP. In some aspects, the additional therapeutic agent is selected from radiation, a cytotoxic agent, a toxin, a chemotherapeutic agent, a cytostatic agent, an anti-hormonal agent, an EGFR inhibitor, an immunomodulatory agent, an anti-angiogenic agent, and combinations thereof. In some embodiments, the additional therapeutic agent is an ABP.

Diagnostic Methods

Also provided are methods for predicting and/or detecting the presence of a given HLA-PEPTIDE on a cell from a subject. Such methods may be used, for example, to predict and evaluate responsiveness to treatment with an ABP and/or cell provided herein.

In some embodiments, a blood or tumor sample is obtained from a subject and the fraction of cells expressing HLA-PEPTIDE is determined. In some aspects, the relative amount of HLA-PEPTIDE expressed by such cells is determined. The fraction of cells expressing HLA-PEPTIDE and the relative amount of HLA-PEPTIDE expressed by such cells can be determined by any suitable method. In some embodiments, flow cytometry is used to make such measurements. In some embodiments, fluorescence assisted cell sorting (FACS) is used to make such measurement. See Li et al., J. Autoimmunity, 2003, 21:83-92 for methods of evaluating expression of HLA-PEPTIDE in peripheral blood.

In some embodiments, detecting the presence of a given HLA-PEPTIDE on a cell from a subject is performed using immunoprecipitation and mass spectrometry. This can be performed by obtaining a tumor sample (e.g., a frozen tumor sample) such as a primary tumor specimen and applying immunoprecipitation to isolate one or more peptides. The HLA alleles of the tumor sample can be determined experimentally or obtained from a third party source. The one or more peptides can be subjected to mass spectrometry (MS) to determine their sequence(s). The spectra from the MS can then be searched against a database. An example is provided in the Examples section below.

In some embodiments, predicting the presence of a given HLA-PEPTIDE on a cell from a subject is performed using a computer-based model applied to the peptide sequence and/or RNA measurements of one or more genes comprising that peptide sequence (e.g., RNA seq or RT-PCR, or nanostring) from a tumor sample. The model used can be as described in international patent application no. PCT/US2016/067159, herein incorporated by reference, in its entirety, for all purposes.

Kits

Also provided are kits comprising an ABP and/or cell provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an ABP provided herein. The label or package insert indicates that the composition is used for treating the selected condition.

In some embodiments, the kit comprises (a) a first container with a first composition contained therein, wherein the first composition comprises an ABP and/or cell provided herein; and (b) a second container with a second composition contained therein, wherein the second composition comprises a further therapeutic agent. The kit in this embodiment can further comprise a package insert indicating that the compositions can be used to treat a particular condition, e.g., cancer.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Generation of Bispecific Antibodies that Specifically Bind an HLA-PEPTIDE Target and CD3

Antigen binding domains specific for various combinations of distinct targets were formatted into six bispecific construct designs (also referred to herein as formats). See International Application No. PCT/US2020/15736 or U.S. application Ser. No. 17/426,627, each of which is hereby incorporated by reference in its entirety. For clarity, for designs (Formats) #2-#6, the antigen binding domains are attached, directly or indirectly, to an Fc region. Format #3, #4, and #5 optionally comprise knob-hole or other Fc heterodimerization modification(s). Format #2 and #6 optionally comprise WT IgG1 Fc sequences without knob-hole modification(s). In some embodiments, target 1 is the HLA-PEPTIDE target and target 2 is a cell surface molecule present on a T cell or NK cell. In some embodiments, target 2 is CD3. The antigen binding domain specific for CD3 can comprise CDRs or variable regions from any anti-CD3 antibody or antigen binding fragment thereof. In some embodiments, target 2 is CD16. In some embodiments, target 1 is an HLA-PEPTIDE target listed in Table A, A1, or A2. In particular embodiments, target one is A*01:01_NTDNNLAVY (SEQ ID NO: 5), A*02:01_LLASSILCA (SEQ ID NO: 8), B*35:01_EVDPIGHVY (SEQ ID NO: 9), A*02:01_AIFPGAVPAA (SEQ ID NO: 6), or A*01:01_ASSLPTTMNY (SEQ ID NO: 7). In more particular embodiments, the antigen binding domain for target 1 (the HLA-PEPTIDE target) comprises CDR sequences from any one of the scFvs specific for A*01:01_NTDNNLAVY (SEQ ID NO: 5), A*02:01_LLASSILCA (SEQ ID NO: 8), B*35:01_EVDPIGHVY (SEQ ID NO: 9), A*02:01_AIFPGAVPAA (SEQ ID NO: 6), or A*01:01_ASSLPTTMNY (SEQ ID NO: 7). In yet more particular embodiments, the antigen binding domain for target 1 (the HLA-PEPTIDE target) comprises the VH and VL sequences from any one of the scFvs specific for A*01:01_NTDNNLAVY (SEQ ID NO: 5), A*02:01_LLASSILCA (SEQ ID NO: 8), B*35:01_EVDPIGHVY (SEQ ID NO: 9), A*02:01_AIFPGAVPAA (SEQ ID NO: 6), or A*01:01_ASSLPTTMNY (SEQ ID NO: 7).

Briefly, bispecific antibodies were generated using standard molecular cloning techniques, including restriction digestion and ligation, gene synthesis, and homology-based cloning methods such as In-fusion (Takara). Positive clones were confirmed by DNA sequencing and used to generate bispecific antibody molecules by transfecting Expi-CHO cells (Thermo) according to the manufacturer's protocol. Cultures were harvested and bispecific antibodies were purified from the supernatants using protein A, Kappa-select, or IMAC (GE healthcare) based chromatography methods. If necessary, bispecific antibodies or controls were polished by SEC or mixed-mode (CHT, BIO-RAD) chromatography. Molecules were formulated in PBS by dialysis or desalting chromatography. Molecules were evaluated to confirm high monomer purity (>95%) and low endotoxin (<1 EU/mg) prior to subsequent testing.

For clarity, the nomenclature of the generated and tested bispecific antibodies recites for Formats #2-#6: as shown in FIG. 76 of International Application No. PCT/US2020/15736 or U.S. application Ser. No. 17/426,627, each of which is hereby incorporated by reference in its entirety; or for format #1 (BiTE): as shown in International Application No. PCT/US2020/15736 or U.S. application Ser. No. 17/426,627, each of which is hereby incorporated by reference in its entirety. Exemplary nomenclatures are shown in FIGS. 77A-C of International Application No. PCT/US2020/15736 or U.S. application Ser. No. 17/426,627, each of which is hereby incorporated by reference in its entirety. For instance, the bispecific designated “1-G2(1H11)-OKT3” is format #1 (BiTE): N-term scFv=G2 clone 1H11, C-term scFv=CD3 binder OKT3. For instance, the bispecific designated “3-G2(1H11)-OKT3” is format #3 (scFv/Fab): scFv=G2(1H11), Fab=OKT3. For yet other instance, the bispecific designated “4-G2(1H11)-OKT3” is format #4 (scFv/scFv-Fab): scFv=G2(1H11), Fab=OKT3.

A list of exemplary bispecific antibodies created using the methods described above is listed in the following table.

TABLE 32
Exemplary bispecific antibodies

| | scFv (N- | scFv (C- | | |
|---|---|---|---|---|
| Format # | term) | term) | scFv | Fab |
| 1. BiTE | G2(1H11) | OKT3 | | |
| 1. BiTE | G7(2E09) | OKT3 | | |
| 1. BiTE | G5(7A05) | OKT3 | | |
| 1. BiTE | G8(2C10) | OKT3 | | |
| 1. BiTE | G2(1H11) | foralumab | | |
| 1. BiTE | G5(7A05) | foralumab | | |
| 1. BiTE | G7(2E09) | foralumab | | |
| 1. BiTE | G8(2C10) | foralumab | | |
| 3. scFv/Fab | | | OKT3 | G2(1H11) |
| 3. scFv/Fab | | | G2(1H11) | OKT3 |
| 3. scFv/Fab | | | G5(7A05) | OKT3 |
| 3. scFv/Fab | | | G7(2E09) | OKT3 |
| 3. scFv/Fab | | | G8(2C10) | OKT3 |
| 3. scFv/Fab | | | G2(1H11) | foralumab |
| 3. scFv/Fab | | | G5(7A05) | foralumab |
| 3. scFv/Fab | | | G7(2E09) | foralumab |
| 3. scFv/Fab | | | G8(2C10) | foralumab |
| 4. scFv/scFv- | | | G2(1H11) | OKT3 |
| Fab | | | | |
| 4. scFv/scFv- | | | G5(7A05) | OKT3 |
| Fab | | | | |
| 4. scFv/scFv- | | | G7(2E09) | OKT3 |
| Fab | | | | |
| 4. scFv/scFv- | | | G8(2C10) | OKT3 |
| Fab | | | | |
| 4. scFv/scFv- | | | G2(1H11) | foralumab |
| Fab | | | | |
| 4. scFv/scFv- | | | G5(7A05) | foralumab |
| Fab | | | | |
| 4. scFv/scFv- | | | G7(2E09) | foralumab |
| Fab | | | | |
| 4. scFv/scFv- | | | G8(2C10) | foralumab |
| Fab | | | | |
| 5. Fc/scFv-Fab | | | G2(1H11) | OKT3 |
| 5. Fc/scFv-Fab | | | G5(7A05) | OKT3 |
| 6. scFv- | | | G2(1H11) | OKT3 |
| Fab/scFv-Fab | | | | |
| 6. scFv- | | | G5(7A05) | OKT3 |
| Fab/scFv-Fab | | | | |
| 2. Fab- | | | G2(1H11) | OKT3 |
| scFv/Fab-scFv | | | | |
| 2. Fab- | | | G5(7A05) | OKT3 |
| scFv/Fab-scFv | | | | |

Amino Acid and nucleotide sequences of exemplary bispecific molecules generated are provided in the Sequences section.

### Example 2: Affinity of Bispecific Formats for the HLA-PEPTIDE Target

Affinity measurements were performed as described herein. Starting antibody concentration was 100 nM and then titrated 1:2 thereafter. The dissociation step in the kinetics buffer was measured for 200 seconds. Data was analyzed using the ForteBio data analysis software using a 1:1 binding model.

Results for the different bispecific formats with the G2(1H11) clone as an ScFv or Fab against HLA-PEPTIDE target A*01:01-NTDNNLAVY (SEQ ID NO: 5) are shown in International Application No. PCT/US2020/15736 or U.S. application Ser. No. 17/426,627, each of which is hereby incorporated by reference in its entirety. All tested bispecific formats exhibited affinity for the HLA-PEPTIDE target, with an apparent KD below 25 nM. The 4-G2(1H11)-OKT3 bispecific shows the highest binding affinity, with an apparent KD of 1.27 nM.

In another set of affinity experiments, starting Fab concentration was 250 nM and titrated 1:2 thereafter. Results for the antibody designated αCD3 (also referred to as anti-CD3) and the hOKT3 IgG are shown in International Application No. PCT/US2020/15736 or U.S. application Ser. No. 17/426,627, each of which is hereby incorporated by reference in its entirety. Both antibodies exhibit binding to CD3 in a dose dependent manner.

Results for the bispecific antibody designated 3-G2(1H11)-hOKT3, 4-G2(1H11)-hOKT3, 2-G2(1H11)-αCD3, 4-G2(1H11)-αCD3, 5-G2(1H11)-αCD3, and 6-G2(1H11)-αCD3 are shown in International Application No. PCT/US2020/15736 or U.S. application Ser. No. 17/426,627, each of which is hereby incorporated by reference in its entirety. The bispecific antibodies exhibited binding to CD3 and HLA-PEPTIDE target A*01:01_NTDNNLAVY (SEQ ID NO: 5) in a dose dependent manner.

### Example 3: Stability of Bispecific Formats

The stability of the bispecific formats was assessed by dynamic light scattering on the Mobius (Wyatt). Samples were stored for 2 months at 4° C. prior to measurement.

The population of the non-aggregated bispecifics at lower calculated radii (<10¹ nm) and any resulting aggregate peak at much higher calculated radii (˜10³ nm) due to instability during storage at 4° C. are shown in International Application No. PCT/US2020/15736 or U.S. application Ser. No. 17/426,627, each of which is hereby incorporated by reference in its entirety. The 4-G2(1H11)-OKT3 bispecific showed the greatest stability, with no aggregate peak detected compared to the other formats.

### Example 4: Tested Bispecific Formats Specifically Bind Cells that Present the HLA-PEPTIDE Target and CD3+ Jurkat Cells

To verify that the generated bispecific antibodies can specifically bind to their HLA-PEPTIDE targets in their natural context, e.g., on the surface of antigen-presenting cells; generated bispecific antibodies specific for G2 and CD3 were used in binding experiments with K562 cells expressing the HLA-PEPTIDE target. Briefly, K562 cells were transduced with HLA-A*01:01 and then pulsed with target or negative control peptide, using the methods described in Example 34. Bispecific binding was detected by flow cytometry.

Results are depicted in International Application No. PCT/US2020/15736 or U.S. application Ser. No. 17/426,627, each of which is hereby incorporated by reference in its entirety. All tested formats exhibited specific binding to HLA-PEPTIDE target G2 (A*01:01_NTDNNLAVY (SEQ ID NO: 5)), with format 4 exhibiting the strongest binding to the target-specific cells.

To verify that the generated bispecific antibodies specifically bind to CD3, the generated bispecific antibodies were used in binding experiments with CD3+ and CD3− Jurkat cells. Results are depicted in International Application No. PCT/US2020/15736 or U.S. application Ser. No. 17/426,627, each of which is hereby incorporated by reference in its entirety. All tested formats exhibited specific binding to CD3+ Jurkat cells but not CD3− Jurkat cells. Comparative results from formats 1, 3, and 4, for the K562 cell binding assay and Jurkat cell binding assay, demonstrating the relative advantages of format 4 were also provided.

### Example 5: Bispecific Antibody 4-G2(1H11)-OKT3 Prevents the Establishment of Tumors in an In Vivo Mouse Model

We established in vivo proof of concept with the format 4 bispecific molecule. The experimental design and conditions of the in vivo experiment is shown in International Application No. PCT/US2020/15736 or U.S. application Ser. No. 17/426,627, each of which is hereby incorporated by reference in its entirety. Briefly, human CD3+ T cells were pre-loaded with the 4-G2(1H11)-OKT3 bispecific and mixed with the A375-10×9mer-Luc tumor cell line, just prior to subcutaneous injection into immunodeficient NSG mice on Day 0. On Day 4, a second dose of the same bispecific was administered.

Results are depicted in International Application No. PCT/US2020/15736 or U.S. application Ser. No. 17/426,627, each of which is hereby incorporated by reference in its entirety. Mice injected with T cells pre-treated with PBS formed tumors as measured by bioluminescence from the A375-10×9mer-Luc tumor cells. However, all mice treated with pre-loaded T cells did not form of any tumor across a range of effector to target ratios (3.5:1, 5:1, and 10:1). Therefore, the bispecific prevented the establishment of tumors expressing the target in vivo in a mouse model.

### Example 6: In Vitro Cytotoxicity for G2 and G5 Lead Bispecific Designs

Materials and Methods

T Cell Activation

For all cytotoxicity assays, negatively selected pan CD3 T cells (AllCells cat #LP, CR, CD3+, NS, 25M) were thawed using dropwise mixing into ImmunoCult media (Stemcell Technologies cat #10981) and activated using ImmunoCult CD3/CD28 activator (Stemcell Technologies #10991) according to manufacturer's instructions. Cells were cultured under standard tissue culture (TC) conditions, 37 deg C., 5% CO₂. 3 days post activation, T cells were checked for activation by visual clumping and used in assays as described below.

Calcein AM Release Cytotoxicity Assay (K562 Cells)

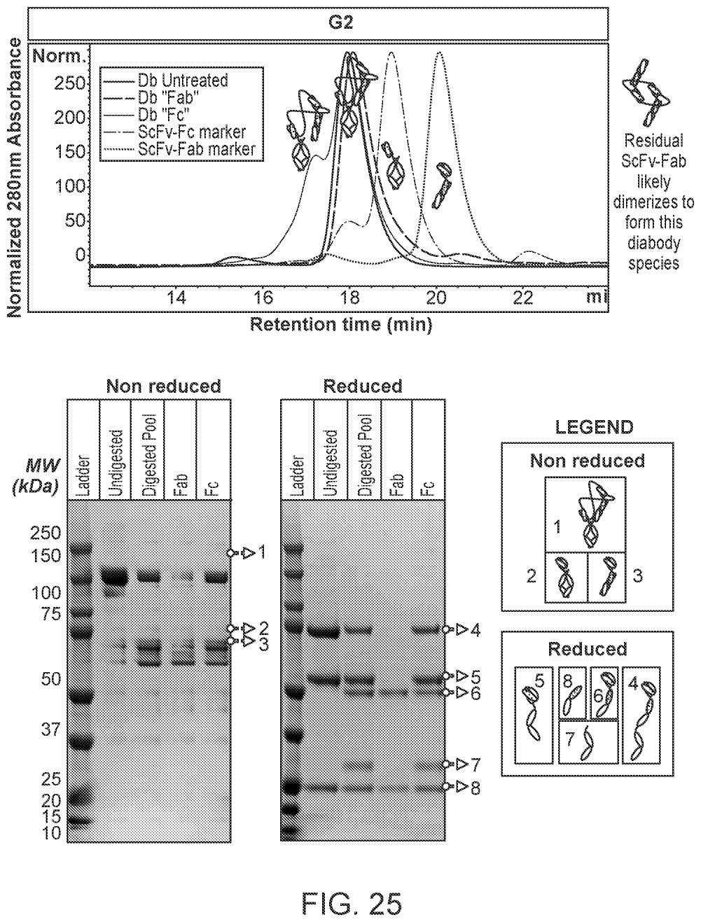

Target cells (K562 cells transduced with the desired HLA and either (1) pulsed with restricted peptide corresponding to the HLA-PEPTIDE target or (2) no restricted peptide control) were pelleted, washed and re-suspended in PBS at 1e7/mL. 1 mM Calcein AM was added and cells incubated for 30 min at 37° C. with mixing every 10 min. Following incubation, cells were pelleted, washed in PBS, and re-suspended at 2e6/mL in serum-free RPMI. 25 μL of target cells were plated in clear TC-treated 96-well U-bottom plates (5e4/well). 25 μL of serially diluted bispecific molecules were added so that final concentrations are as indicated in figures. 25 μL of T cells, washed and re-suspended at 2e7/mL in serum-free RPMI, were added to plates to give a 10:1 T cell:target cell ratio. RPMI-only, target cell only, and 1% triton-lysed cells were included to measure background, spontaneous and maximum release, respectively. Plates were incubated for 6 hours under standard TC conditions. Following incubation, plates were spun down at 300 g for 5 minutes. 60 μL of supernatant (SN) were transferred to opaque black 96 well plates. Fluorescence intensity (495 nm) was measured on a SpectraMax plate reader using SoftMax Pro software. To calculate % killing, RPMI background was first subtracted from all values. % killing was determined using % cytotoxicity w/ Ab−% cytotoxicity w/o Ab. % cytotoxicity was calculated as [(A−B)/(C−B)]×100, where A=experimental release, B=spontaneous release, C=maximal release.

Luciferase Cytotoxicity Assay (A375 G2 Cells)

A375 cells, which express HLA-A*01:01, were engineered to express the restricted peptide NTDNNLAVY (SEQ ID NO: 5) using a lentivirus transduction of a cassette containing a 10× repeat of the peptide, Luciferase, and puromycin-resistance. Cassette-expressing cells were selected using 0.5 μg/mL of puromycin. For the assay, cells were pelleted, washed in PBS, and re-suspended at 2e6/mL in RPMI with 10% FBS. 25 μL of target cells were plated in opaque white 96-well plates. Serial dilutions of the bispecific molecules were added as described above. T cells were added to the plates to give a 10:1 T cell:target ratio as described above. Following 24-hour incubation, Bio-Glo luciferase substrate (Promega cat #G7941) was added and plate incubated and read according to manufacturer's instructions. To calculate % killing, RPMI background RLU was first subtracted from all values. % killing was determined as % cytotoxicity w/ Ab−% cytotoxicity w/o Ab, where % cytotoxicity was calculated as 100%-% viability. % viability was calculated as % of RLU in experimental wells normalized against target cells alone.

LDH Release Cytotoxicity Assay (A375 G2 Cells)

Plates contained serial dilutions of the bispecific molecules and 10:1 T cell:target ratio as described above and incubated for 48 h in clear TC-treated 96w U-bottom plates. Plates were spun down at 300 g×5 min, and supernatant removed and diluted 1:100. LDH-Glo assay kit was used (Promega cat #J2381) and % killing calculated according to manufacturer's instructions.

Results

FIG. 86A in International Application No. PCT/US2020/15736 (which is hereby incorporated by reference in its entirety) depicts the bispecific formats tested for the 01:01 NTDNNLAVY (SEQ ID NO: 5) T cell redirecting bispecific binding molecules. The binding domain(s) specific for *01:01_NTDNNLAVY (SEQ ID NO: 5) were from the G2(1H11) clone. The binding domain specific for CD3 were from CD3 antibody OKT3. Calcein AM cytotoxicity results for the A*01:01_NTDNNLAVY (SEQ ID NO: 5)/CD3 bispecific molecules, in various bispecific formats are shown in International Application No. PCT/US2020/15736 or U.S. application Ser. No. 17/426,627, each of which is hereby incorporated by reference in its entirety. All formats induced cytotoxicity in the K562 cells expressing HLA-PEPTIDE target A*01:01_NTDNNLAVY (SEQ ID NO: 5), relative to K562 cells not expressing the HLA-PEPTIDE target (unpulsed controls). Redirecting bispecific molecules in BiTE format (format #1) and the scFv/scFv-Fab format (format #4) induced greater cytotoxicity as compared to the scFv/Fab format (format #3).

FIG. 87A in International Application No. PCT/US2020/15736 or U.S. application Ser. No. 17/426,627, each of which is hereby incorporated by reference in its entirety depicts the bispecific formats tested for the B*35:01_EVDPIGHVY (SEQ ID NO: 9) T cell redirecting bispecific binding molecules. The binding domain(s) specific for B*35:01_EVDPIGHVY (SEQ ID NO: 9) were from the G5(7A05) clone. The binding domain specific for CD3 were from CD3 antibody OKT3. Calcein AM cytotoxicity results for the B*35:01_EVDPIGHVY (SEQ ID NO: 9)/CD3 bispecific molecules, in various bispecific formats are shown in International Application No. PCT/US2020/15736 or U.S. application Ser. No. 17/426,627, each of which is hereby incorporated by reference in its entirety. All formats induced cytotoxicity in the K562 cells expressing HLA-PEPTIDE target B*35:01_EVDPIGHVY (SEQ ID NO: 9), relative to K562 cells not expressing the HLA-PEPTIDE target (unpulsed controls). Redirecting bispecific molecules in BiTE format (format #1) and the scFv/scFv-Fab format (format #4) induced greater cytotoxicity as compared to the scFv/Fab format (format #3).

Results from the luciferase assay in A375 cells are shown in International Application No. PCT/US2020/15736 or U.S. application Ser. No. 17/426,627, each of which is hereby incorporated by reference in its entirety. Bispecific molecules that bind *01:01 NTDNNLAVY (SEQ ID NO: 5) and CD3 were tested. The binding domains specific for *01:01 NTDNNLAVY (SEQ ID NO: 5) were from the G2(1H11) clone. The binding domains specific for CD3 were from CD3 antibody OKT3. As demonstrated, the highest dose caused cellular cytotoxicity for all bispecific formats tested. The serial dilution curves demonstrate that Format #4 (scFv/scFv-Fab) exhibited the strongest dose-response curve out of the three formats, followed by Format #1 (BiTE), followed by Format #3 (scFv/Fab).

Additional results from a second round of the luciferase assay in A375 cells are shown in International Application No. PCT/US2020/15736 or U.S. application Ser. No. 17/426,627, each of which is hereby incorporated by reference in its entirety. Bispecific molecules that bind A*01:01_NTDNNLAVY (SEQ ID NO: 5) and CD3 were tested. The binding domains specific for A*01:01_NTDNNLAVY (SEQ ID NO: 5) were from the G2(1H11) clone. The binding domains specific for CD3 were from an anti-CD3 antibody or CD3 antibody hOKT3. As demonstrated, all formats induced cytotoxicity in a dose-dependent manner. As demonstrated, the highest dose caused cellular cytotoxicity for formats 3 and 4. In particular, format #4 of the bispecific antibody G2(1H11)-hOKT3 resulted in a high levels of cytotoxicity across all concentrations tested.

Results from the LDH assay in A375 cells are shown in International Application No. PCT/US2020/15736 or U.S. application Ser. No. 17/426,627, each of which is hereby incorporated by reference in its entirety. At the highest dose, all tested formats induced cytotoxicity, with Formats #1 (BiTE) and #4 (scFv/scFv-Fab) showing higher cytotoxicity as compared to the Format #3 (scFv/Fab) bispecific antibodies.

### Example 7: Bispecific Antibodies Bind Cells that Present the HLA-PEPTIDE Target and CD3+ Jurkat Cells

After reformatting our TRCm antibody into various bispecific formats, we tested their ability to bind the specific pHLA target as well as CD3+ Jurkats. See sequence tables labeled Exemplary Bispecific Format 1 Constructs, Exemplary Bispecific Format 2 Constructs, Exemplary Bispecific Format 3 Constructs, Exemplary Bispecific Format 4 Constructs, Exemplary Bispecific Format 5 Constructs, and Exemplary Bispecific Format 6 Constructs for amino acid sequence information of the tested bispecific antibodies. Therefore, we conducted titration experiments on K562 cells that were transduced HLA-A*01:01 and exogenously pulsed with target or negative control peptide. Target specific binding was also tested on A375 cells transduced with high or medium levels of target as well as A375 transduced with control construct. Bispecific binding was detected by flow cytometry.

Materials and Methods

K562 cell lines were generated as described in Example 34.

A375 cell lines, which express HLA-A*01:01, were engineered to express the restricted peptide NTDNNLAVY (SEQ ID NO: 5) as described in Example 6.

Flow Cytometry Methods:

HLA-transduced K562 cells were pulsed as described in Example 34. Cells were harvested, washed in PBS, and stained with eBioscience Fixable Viability Dye eFluor 450 for 15 minutes at room temperature. Following another wash in PBS+2% FBS, cells were resuspended with bispecifics at varying concentrations. Cells were incubated with bispecifics for 1 hour at 4° C. After another wash, PE-conjugated goat anti-human IgG secondary antibody (Jackson ImmunoResearch) was added at 1:100, or anti-His Alexa Fluor 647 (BioRad) at 1:20 for detection of the BiTE molecule. After incubating at 4° C. for 45 minutes and washing in PBS+2% FBS, cells were resuspended in PBS+2% FBS and analyzed by flow cytometry. Jurkat E6-1 (ATCC TIB-152) and Jurkat T3.5 (ATCC TIB-153) cells were grown under standard tissue culture conditions. All cell lines were stained and analyzed with bispecific binding using the same methods as the K562 cells.

Flow cytometric analysis was performed on the Attune NxT Flow Cytometer (ThermoFisher) using the Attune NxT Software. Data was analyzed using FlowJo.

Results

K562 binding results for bispecific formats of clone G2(1H11) with an anti-CD3 arm are shown in International Application No. PCT/US2020/15736 or U.S. application Ser. No. 17/426,627, each of which is hereby incorporated by reference in its entirety. Bispecifics in formats 2-6 exhibited specific binding to K562 cells pulsed with target restricted peptide, as compared to K562 cells pulsed with a known off target peptide (YSEHPTFTSQY (SEQ ID NO: 401)) or unpulsed controls.

A375 binding results for bispecific formats of clone G2(1H11) with an anti-CD3 arm are shown in International Application No. PCT/US2020/15736 or U.S. application Ser. No. 17/426,627, each of which is hereby incorporated by reference in its entirety. Low MOI refers to low antigen expression, high MOI refers to high antigen expression. Low and high antigen expression was achieved as described in Example 8.

Jurkat binding results for bispecific formats of clone G2(1H11) with an anti-CD3 arm are shown in International Application No. PCT/US2020/15736 or U.S. application Ser. No. 17/426,627, each of which is hereby incorporated by reference in its entirety. Bispecifics in formats 2-6 exhibited specific binding to CD3+ Jurkat cells as compared to CD3− Jurkat cells.

K562 binding results for bispecific formats of clone G2(1H11) with an hOKT3 arm are shown in International Application No. PCT/US2020/15736 or U.S. application Ser. No. 17/426,627, each of which is hereby incorporated by reference in its entirety. Bispecifics in formats 3 and 4 exhibited specific binding to K562 cells pulsed with target restricted peptide, as compared to K562 cells pulsed with a known off target peptide (YSEHPTFTSQY (SEQ ID NO: 401)) or unpulsed controls.

A375 binding results for bispecific formats of clone G2(1H11) with an hOKT3 arm are shown in International Application No. PCT/US2020/15736 or U.S. application Ser. No. 17/426,627, each of which is hereby incorporated by reference in its entirety. Low MOI refers to low antigen expression, high MOI refers to high antigen expression. Low and high antigen expression was achieved as described in Example 8.

All formats tested bind in a dose-dependent manner that is selective for the relevant target peptide on all cells. In addition, all formats bind to CD3+, but not CD3−, Jurkat cell lines, indicating that this interaction is made through the anti-CD3 portion of the bispecific molecules.

### Example 8: Bispecific Antibodies Induced T-Cell Mediated Cytotoxicity of Tumor Cell Lines Expressing HLA-PEPTIDE Targets in a Spheroid Toxicity Model

Bispecific antibodies to various HLA-PEPTIDE targets, carrying various anti-CD3 binding domains, were tested in a spheroid cytotoxicity model. See sequence tables labeled Exemplary Bispecific Format 1 Constructs, Exemplary Bispecific Format 2 Constructs, Exemplary Bispecific Format 3 Constructs, Exemplary Bispecific Format 4 Constructs, Exemplary Bispecific Format 5 Constructs, and Exemplary Bispecific Format 6 Constructs for amino acid sequence information of exemplary tested bispecific antibodies. Also tested (full chain sequence data not shown) were formats #1-#6 using foralumab as the antigen binding domain specific for CD3. When grown in low attachment plates, cancer cell lines aggregate into spheroid bodies, which more closely mimic three dimensional tumors as compared to cell lines grown under adherent conditions. See, e.g., SLAS Discovery 2017, Vol. 22(5) 456-472, which is hereby incorporated by reference in its entirety.

Materials and Methods

Cell Lines

The cell lines used to express the desired HLA-PEPTIDE targets were as follows: A375 cells (which express HLA subtype A*01:01) engineered to express the G2 restricted peptide NTDNNLAVY (SEQ ID NO: 5), LN229 (which express HLA subtype B*35:01) engineered to express the G5 restricted peptide EVDPIGHVY (SEQ ID NO: 9); and A375 (which also express HLA subtype A*02:01) engineered to express the G8 restricted peptide AIFPGAVPAA (SEQ ID NO: 6). All cell lines were also engineered to express luciferase.

Luciferase expressing cells were plated in 100 μL at 10,000-15,000 cells/well in Corning ultra-low attachment plates (Corning #4515) in corresponding culture medium without selection. Plates were incubated for two days at 37° C. and 5% CO2 to allow spheroid formation. Antibody was titrated at and added as 10 μL/well. Normal human PBMCs were thawed and rested for 4-6 hours at 37° C. and added as 100,000 cells/well in 50 μL giving an Effector:Target ratio of 10:1. Plates were then incubated for 4 days at 37° C. and 5% CO2. At the end of the incubation period 100 μL Luciferin (Pierce #88292) at 300 μg/mL was added to the plate. Luciferase was read on the SpectraMax iE3. Percent cytotoxicity was calculated as (Media control−sample signal)/(Media control−maximum lysis)*100.

Results for G2(1H11)-αCD3 bispecific antibodies in various formats are shown in International Application No. PCT/US2020/15736 or U.S. application Ser. No. 17/426,627, each of which is hereby incorporated by reference in its entirety also shows maximum and minimum cytotoxicity, as well as IC50 data, for bispecifics for which dose-response curves were generated. Formats 6 and 4 were the most potent in inducing cytotoxicity, each inducing similar high levels of maximum cell killing. Format 2 also induced cytotoxicity to a higher level than αCD3 alone. However, Format 5 did not induce increased cell killing as compared to αCD3 alone.

Also shown are Format 2, 4, and 6 bispecific dose-response curves for A375 cells engineered to express low and high levels of the G2 restricted peptide. Differing levels of antigen expression were achieved by transduction with varying titers of virus and selection of different clonal cell lines by limiting dilution. Results show that the bispecifics induce cytotoxicity in a dose-dependent manner. At low levels of G2 expression, Format 6 was the most potent in inducing cytotoxicity, followed by Format 4, then Format 2. However, both formats 4 and 6 induced similar maximum levels of cytotoxicity at the highest dose. At high levels of G2 expression, Formats 4 and 6 exhibited similar high potency, followed by Format 2.

Results for G8(2C10)-foralumab bispecific antibodies in formats #2 and #3 are shown in International Application No. PCT/US2020/15736 or U.S. application Ser. No. 17/426,627, each of which is hereby incorporated by reference in its entirety. Both formats enhanced T-cell mediated cytotoxicity in a dose-dependent manner, as compared to G8(2C10) IgG.

Results for G5(7A05) bispecific antibodies with foralumab or hOKT3 arms tested against the engineered LN229 cell line are shown in International Application No. PCT/US2020/15736 or U.S. application Ser. No. 17/426,627, each of which is hereby incorporated by reference in its entirety. Format 2 with the foralumab arm exhibited the highest potency, followed by format 1-foralumab, followed by format 1-OKT3.

### Example 9: SEC-HPLC Analysis of Format 4 Bispecific Antibodies Reveal Presence of an Alternative Isomer

Methods

Analytical SEC-HPLC was performed on an Agilent 1200 series HPLC system equipped with a degasser (G1379B), binary pump (G1312B), high performance autosampler (G1367D), and wide range diode array detector (DAD, G7115A). Approximately 50-100 ug of Format 4 G5(1C12) protein A eluate, neutralized to pH 7 using 1M Tris buffer pH 7.5, was loaded onto a TSKgel SuperSW mAb HTP column (4.6 mm ID×15 cm) with the TSKgel Guardcolumn SuperSW mAb guard column in line, or TSKgel G3000 SWxl column (7.8 mm ID×30 cm) with the TSKgel G2000SWxl-G4000SWxl Guard Column in line from Tosoh Bioscience. The TSKgel SuperSW mAb HTP column was operated at 0.35 ml/min for 7 min in PBS pH 7.4. The TSKgel G3000 SWxl column was operated at 0.5 ml/min for 35 min in PBS, pH 7.4. The DAD was set to collect absorbance at 280 nm for both methods.

Results:

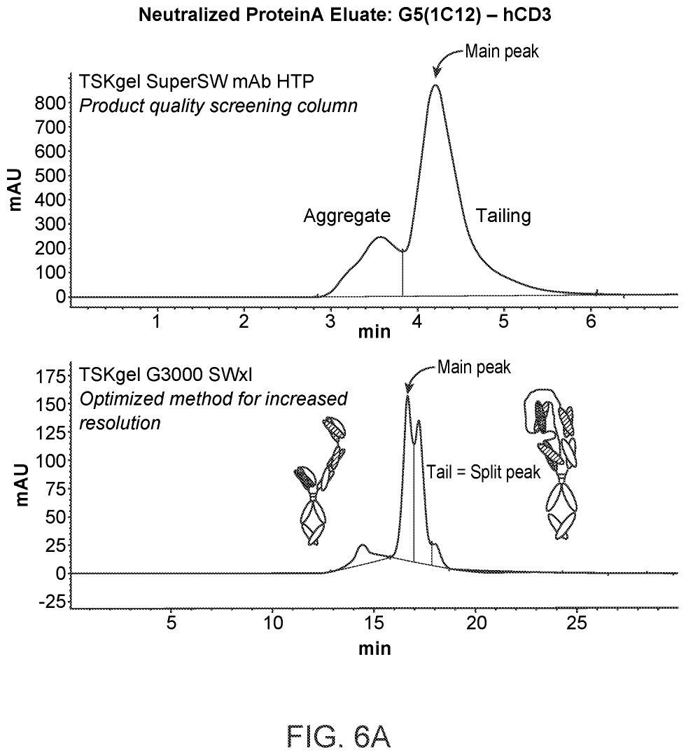

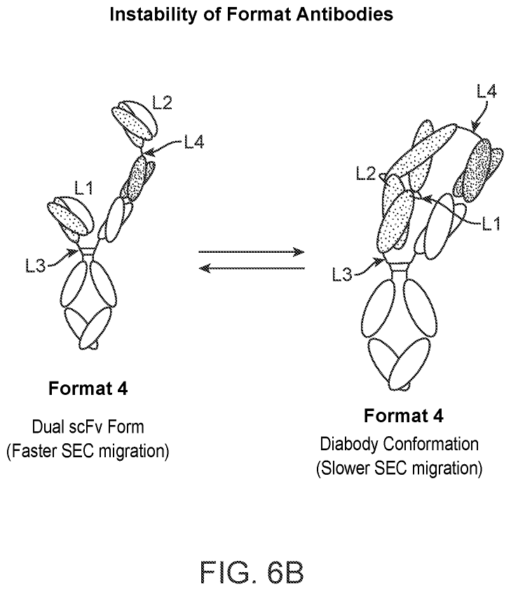

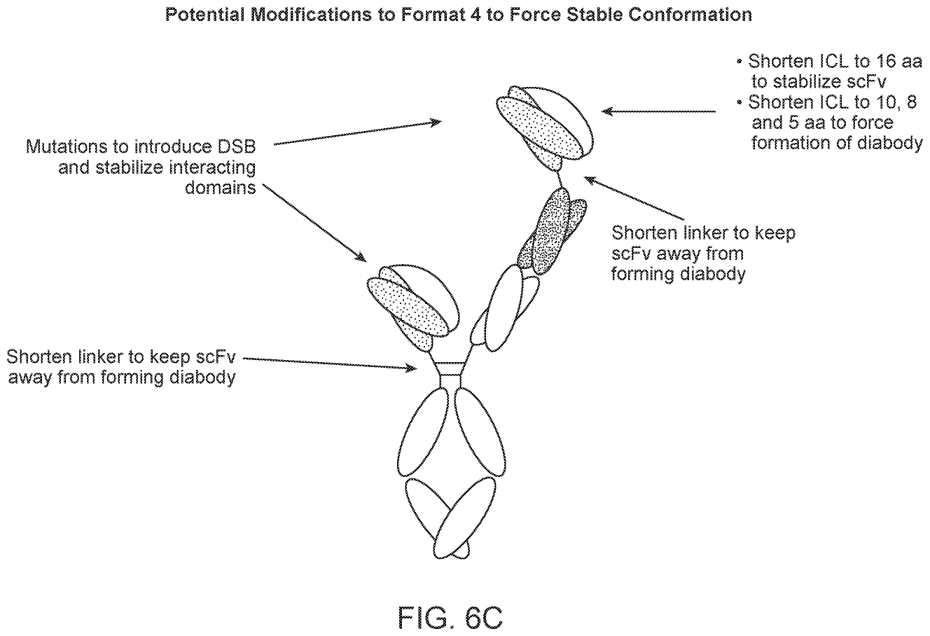

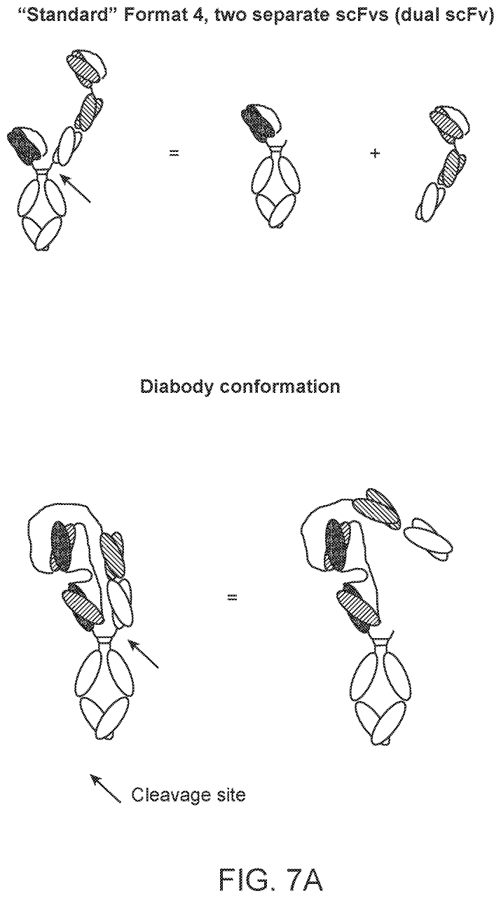

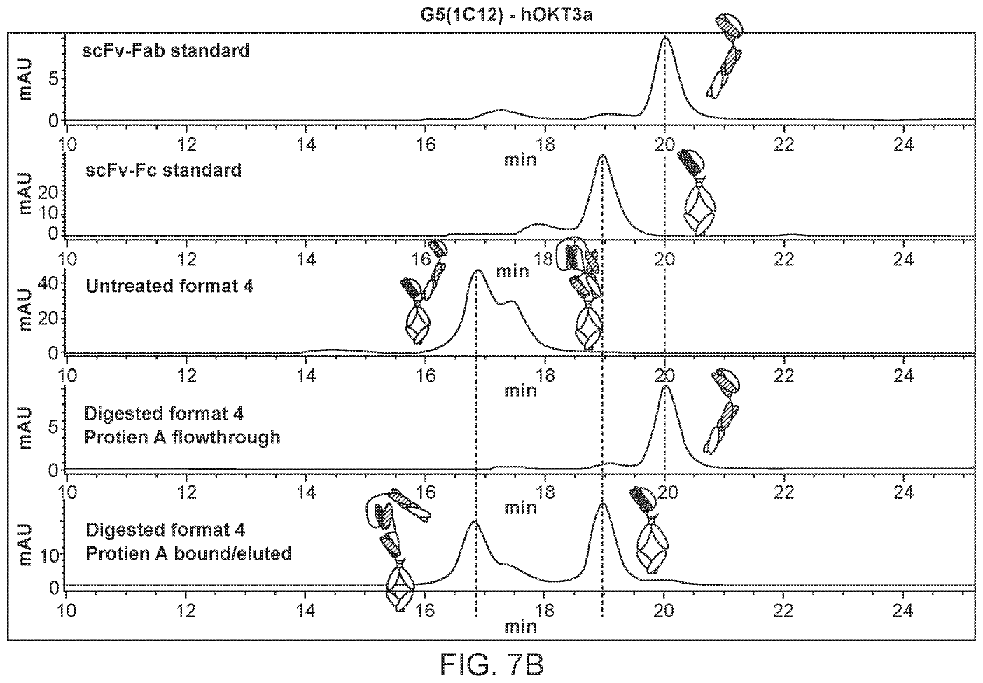

Analysis of Format 4 G5(1C12) proteinA eluate using the TSKgel SuperSW mAb HTP column (FIG. 6A, top), used for quick product quality screening of antibodies, revealed the presence of aggregates between 3-4 min, a main peak, and an unexpected significant tailing between 4.5-5.5 minutes. The observed tailing suggested the presence of an additional antibody moiety that either interacts more with the SEC column, or is more compacted and thus migrates slower than the main antibody conformation. Analyzing the same proteinA eluate using the TSKgel G3000SWxl column, which has greater resolving power than the shorter TSKgel SuperSW mAb HTP column, shows that the tailing initially observed resolves into a “split peak” (FIG. 6A, bottom). Mass spectrometry analysis of the G5(1C12) Format 4 antibody suggested no fragmentation (data not shown). Accordingly, the “split peak” was hypothesized to be a diabody isoform of the Format 4 antibody, where the VH of one of the scFvs interacts with the VL of the other scFv and vice versa. (FIG. 6A, bottom panel, bispecific diagram on the right).

These results indicated that the antibodies can exist in two conformations. They further indicated that in solution, the antibody will be in equilibrium between (1) a dual scFv conformation and (2) a diabody conformation (see FIG. 6B).

### Example 10: Determination of Alternate Diabody Isoform

Materials and Methods

Antibody Digestion Experiment

0.4 mg each of purified G5(1C12) format 3, 4 and 5 bispecific antibodies were buffer exchanged from PBS pH 7.4 into 150 mM sodium phosphate buffer at pH 7.0. The samples were then concentrated to a volume of approximately 100 μL, with corresponding concentrations ranging from 3-4 mg/mL, loaded onto FabALACTICA microspin columns (Genovis), and incubated for 16 hr with end-over-end mixing. FabALACTICA antibody digestion involves a cysteine protease that digests human IgG1 at one specific site above the hinge (KSCDKT/HTCPPC (SEQ ID NO: 2)), generating intact Fab and Fc fragments. The name of the enzyme is derived from the pathogen Streptococcus agalactiae, where it was first discovered. Spoerry, Christian & Hessle, Pontus & Lewis, Melanie & Paton, Lois & Woof, Jenny & Pawel-Rammingen, Ulrich. (2016). Novel IgG-Degrading Enzymes of the IgdE Protease Family Link Substrate Specificity to Host Tropism of Streptococcus Species. PLoS ONE. 11. e0164809. 10.1371/journal.pone.0164809), which is hereby incorporated by reference in its entirety. To collect the digested products, the columns were centrifuged at 1000×g for 1 min, followed by two additional rounds of elution using 100 μL PBS pH 7.4. The elution fractions were pooled and subsequently loaded onto a CaptureSelect (Genovis) column, and incubated for 30 min with end-over-end mixing. The flowthrough was collected by centrifugation at 200×g for 1 min, followed by two wash steps with 100 μL PBS (200×g for 1 min, and 100×g for 1 min, respectively). The flowthrough and wash fractions were pooled, and are henceforth referred to as “ProteinA Flowthrough”. The ProteinA bound fragments were eluted using 100 μL of 0.1M Glycine, pH 3 by centrifugation at 200×g for 1 min, and immediately neutralized with 50 μL 1M tris pH 7.5. A second elution step was performed by centrifugation at 1000×g for 1 min, and neutralized immediately as described. The elution fractions were pooled and are henceforth referred to as “ProteinA bound/Eluted”

SEC-HPLC Analysis

Analytical SEC-HPLC was performed on an Agilent 1200 series HPLC system equipped with a degasser (G1379B), binary pump (G1312B), high performance autosampler (G1367D), and wide range diode array detector (DAD, G7115A. Approximately 40 μL of each of untreated antibody, digested proteinA flowthrough, and digested ProteinA bound/eluted was loaded onto a TSKgel G3000 SWxl column (7.8 mm ID×30 cm) with the TSKgel G2000SWxl-G4000SWxl Guard Column in line from Tosoh Bioscience. The column was operated at 0.5 ml/min for 60 min in PBS, pH 7.4. The DAD was set to collect absorbance at 280 nm.

Results

FIG. 7A depicts expected protein digestion fragments of “standard” Format 4 antibodies and a “diabody” isomer of Format 4. FabALACTICA digestion of “standard” Format 4 conformation (scFv/scFv-Fab) antibodies with two separate scFvs, without presence of any alternative “diabody” isoforms, would be expected to yield two peaks: one corresponding to the scFv-Fc fragment and one corresponding to the scFv-Fab fragment. Presence of a Format 4 “alternative diabody” conformation would be expected to reveal presence of a third peak that aligns with the undigested Format 4 main peak.

SEC-HPLC results are depicted in FIG. 7B. Digested format 5 ProteinA flowthrough is used as the ScFv Fab standard, and digested format 3 Protein A bound/Eluted is used as the ScFv-Fc standard. The undigested format 4 SEC-HPLC profile shows the previously described split peak. Digested format 4 ProteinA flowthrough showed a peak with a retention time that aligned with the ScFv-Fab standard. Digested format 4 ProteinA bound/Eluted SEC-HPLC profile showed a peak that aligned with the ScFv-Fc standard expected to be seen for the “standard” Format 4, as well as a peak that aligned with the undigested format 4. The presence of the latter peak indicated the presence of the alternate diabody conformation.

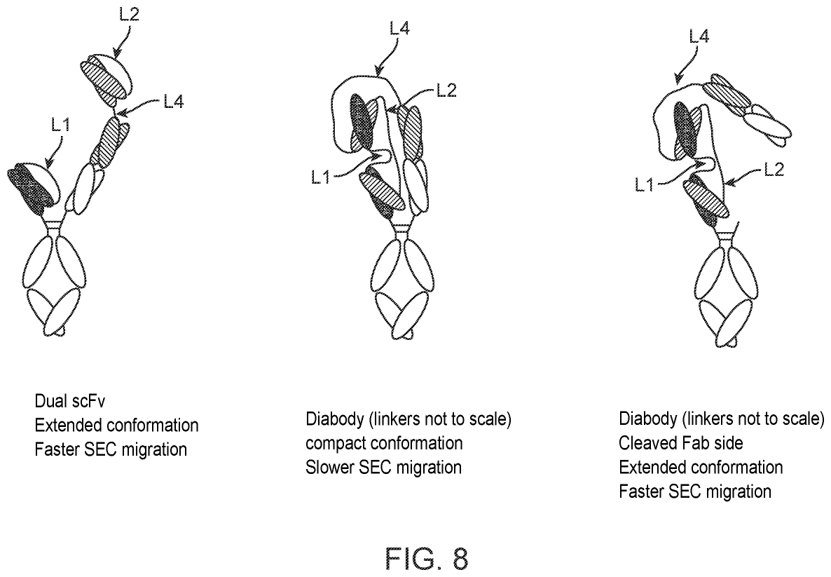

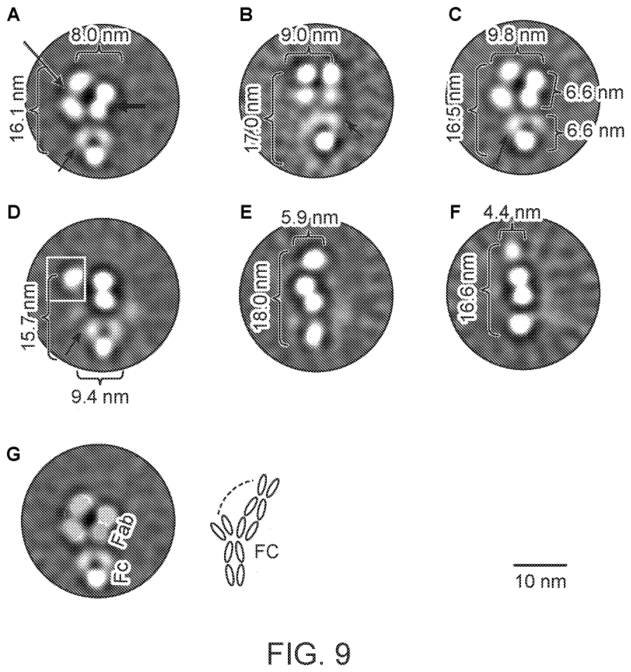

FIG. 8 depicts a diagram representation of the undigested Format 4 “separate scFv” conformation (left), the alternate diabody conformation without digestion (middle), and the alternate diabody conformation with digestion (right).

### Example 11: Negative Stain Electron Microscopy and 2D Class Averaging

Materials and Methods

Grid Preparation

A sample of Format 4-hOKT3-G5(1C12) bispecific antibody was diluted to 18 μg/mL using PBS prior to imaging. The sample was imaged over a layer of continuous carbon supported by nitro-cellulose on a 400-mesh copper grid. The grids were prepared by applying 3 of sample suspension to a cleaned grid, blotting away with filter paper, and immediately staining with uranyl formate.

EM Imaging

Electron microscopy was performed using an FEI Tecnai T12 electron microscope (serial number D1100), operating at 120 keV equipped with an FEI Eagle 4k×4k CCD camera. Negative stain grids were transferred into the electron microscope using a room temperature stage.

Images of each grid were acquired at multiple scales to assess the overall distribution of the specimen. After identifying potentially suitable target areas for imaging at lower magnifications, high magnification images were acquired at nominal magnifications of 110,000× (0.10 nm/pixel) and 67,000× (0.16 nm/pixel). The images were acquired at a nominal underfocus of −1.6 μm to −0.8 μm and electron doses of ˜25 e/Å.

2D Averaging Analysis

Particles were identified in the high magnification images prior to alignment and classification. The individual particles were then selected, boxed out, and individual sub-images are combined into a stack to be processed using reference-free classification.

Particle Selection: Individual particles in the 67,000× high magnification images were selected using automated picking protocols described in Lander, G. C., S. M. Stagg, et al. (2009). “Appion: an integrated, database-driven pipeline to facilitate EM image processing.” J Struct Biol 166(1): 95-102, which is hereby incorporated by reference in its entirety, and manual picking. An initial round of alignments was done on each sample and from that alignment class averages that appeared to contain recognizable particles were selected for additional rounds of alignment.

Particle Alignment and Classification: A reference-free alignment strategy based on the XMIPP (Sorzano, Marabini et al. 2004) processing package, described in Sorzano, C., R. Marabini, et al. (2004). XMIPP: a new generation of an open-source image-processing package for electron microscopy. J Struct Biol. 148: 194-204, which is hereby incorporated by reference in its entirety, was used. Algorithms in this package align the selected particles and sort them into self-similar groups of classes.

Results

FIG. 9 depicts electron microscopy results. Visible in the sample were particles that displayed different sizes and morphologies. Particles ranged from ˜16-22 nm in their longest dimension and had a wide range of conformations; some particles had a branched appearance and others were irregular in shape. Class averages showed particles that ranged from ˜5 to 10 nm in width and ˜16 to 18 nm in length (see FIG. 9). The majority of the class averages contained features that resembled those seen for IgG molecules: a single Fc domain and two antibody arms. However, there were aspects that distinguished these particles from a typical antibody sample: 1. One of the antibody arms contained a peanut-shaped moiety closely resembling a typical Fab (FIG. 9, panel A, black arrow). The other arm appeared to contain two spherical domains, but at a greater distance from each other when compared to that seen in a standard Fab arm (FIG. 9A, panel A, light gray arrow). Based on the model of this bispecific antibody, it is likely that only one of these two spherical domains was connected to the Fc region, whereas the other was in fact connected to the end of the neighboring arm. It seems to be flexibly linked, as it can bend down and interact with the tip of the neighboring Fab arm. These interacting spherical domains are mostly likely the two scFv domains of the Format 4 antibody. Thus the EM revealed visual evidence of the alternative diabody isomer.

It should be noted that in a few class averages, the Fc and Fab domains were stacked in a straight line making it impossible to distinguish between them (FIG. 9, panels E and F). These are likely side views of the particle described above.

Averages were generally well-defined, with some portions of the Fc domain not as clearly resolved as others.

### Example 12: Introduction of DSB44/100 Removes Putative Diabody Peak

Materials and Methods

DSB Engineering

Position 44 of the VH (Kabat) is often in close proximity to position 100 of VL (Kabat). By introducing Cys residues at both of these positions, a disulfide bond (DSB) can be formed that stabilizes the VH/VL interactions within each scFv, prior to assembly of the bispecific antibody chains. Such a stabilizing DSB would be expected to reduce the probability that the two scFvs of the Format 4 antibodies interact to form the alternative diabody isomer.

Gene fragments incorporating the H44-L100 DSB mutations (Kabat numbering) were ordered through Genewiz, incorporating 18-base pair overlaps with digested vector. Fragments were cloned using In-Fusion homologous recombination (Takara) according to manufacturer's instructions. Clones were confirmed to be correct by sequencing (Elim Biopharmaceuticals). Molecules were generated by transfection of Expi293F cells according to manufacturer's recommended protocols (Life Technologies). Molecules were purified on Akta AVANT using protein A and Kappa Select Light columns (GE Healthcare) and polished using CHT (Bio-Rad) for aggregate removal.

SEC-HPLC

Analytical SEC-HPLC was performed on an Agilent 1200 series HPLC system equipped with a degasser (G1379B), binary pump (G1312B), high performance autosampler (G1367D), and wide range diode array detector (DAD, G7115A). Purified Format 4 G5(1C12) and G2(1H11) antibodies, with and without the DSB were loaded onto a TSKgel G3000 SWxl column (7.8 mm ID×30 cm) with the TSKgel G2000SWxl-G4000SWxl Guard Column in line from Tosoh Bioscience. The column was operated at 0.5 ml/min for 30 min in PBS, pH 7.4. The DAD was set to collect absorbance at 280 nm.

Results

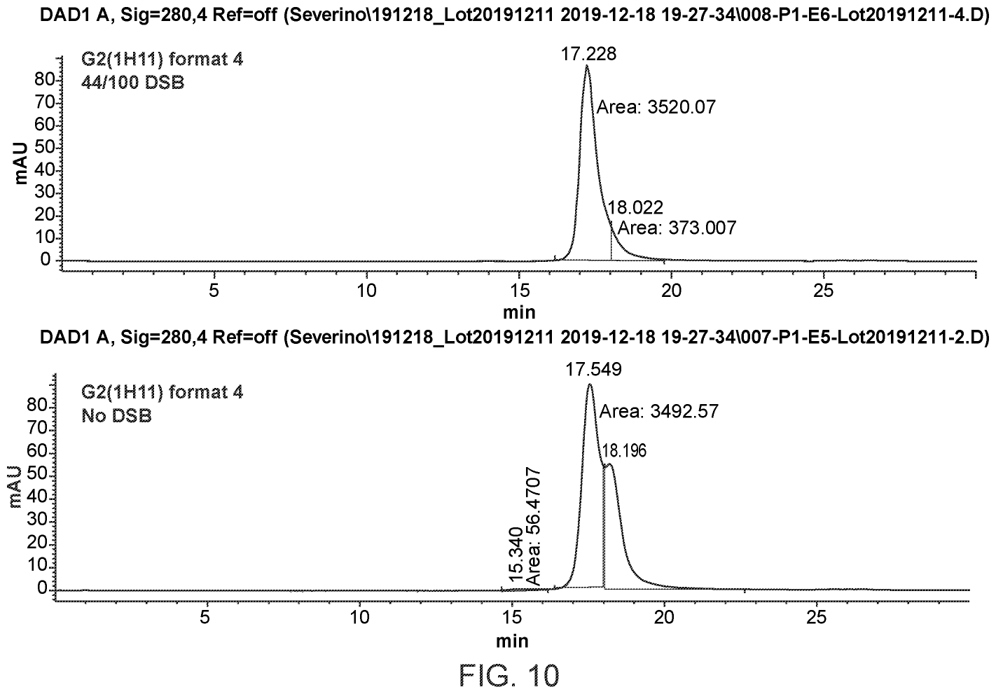

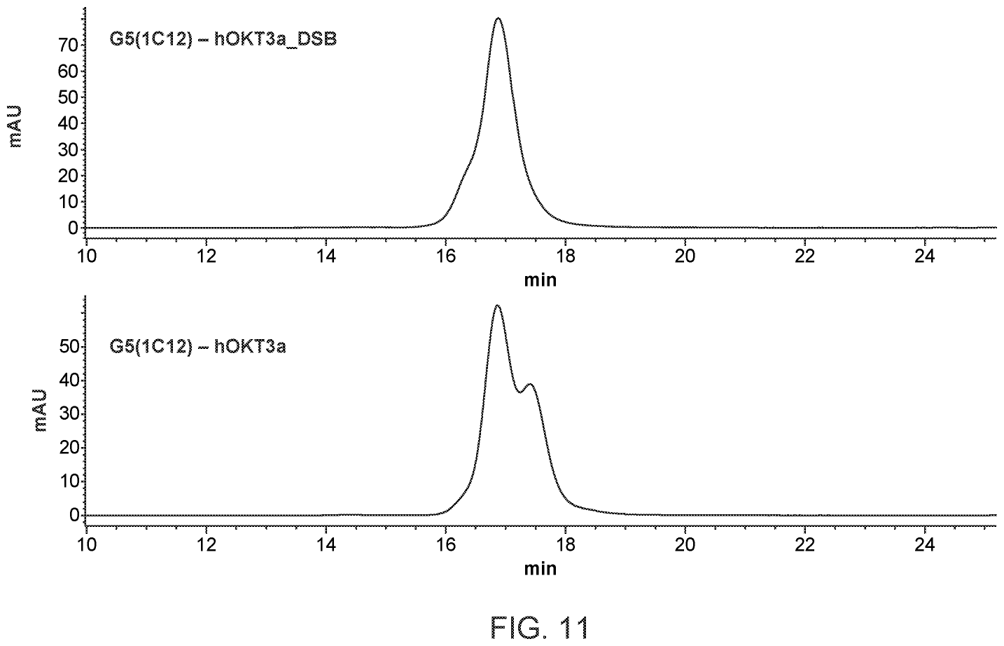

Bottom panels of FIG. 10 and FIG. 11 show the previously observed split peak for both Format 4 G5 and G2 molecules, indicating the presence of both “standard” (with two separate scFvs) and alternate diabody conformation across all Format 4 molecules. Introduction of a stabilizing disulfide bond within the scFv regions of both molecules is shown to remove the split peak (top panels of FIG. 10 and FIG. 11). A retention time that aligns with that of the Format 4 “standard” conformation suggests that the introduction of a disulfide bond stabilizes the standard conformation with two separate scFvs for both G5 and G2 molecules and reduces their isomerization into the alternative diabody format.

### Example 13: Digestion of Format 4 Antibody with DSB44/100

Materials and Methods:

Proteolysis by FabALACTICA:

0.3-0.5 mg of G5(1C12)-hOKT3_DSBH44/L100 Format 4 and G2(1H11)-hOKT3_DSBH44/L100 Format 4 antibodies (both having non-shortened linkers: L1=L2=(G4S)×4 (SEQ ID NO: 3); L3=L4=(G4S)×2) (SEQ ID NO: 4) were buffer exchanged from PBS pH 7.4 into 150 mM sodium phosphate buffer at pH 7.0. The samples were then concentrated to a volume of approximately 100 μL, with corresponding concentrations ranging from 3-5 mg/mL, loaded onto FabALACTICA microspin columns (Genovis), and incubated for 18 hr with end-over-end mixing. To collect the digested products, the columns were centrifuged at 1000×g for 1 min, followed by three additional rounds of elution using 100 μL PBS pH 7.4. The elution fractions were pooled (referred to as “digested pool”), and subsequently loaded onto a CaptureSelect ProteinA (Genovis) column, and incubated for a minimum of 30 min with end-over-end mixing. The flowthrough was collected by centrifugation at 200×g for 1 min, followed by three wash steps with 100 μL PBS pH 7.4. The flowthrough and wash fractions were pooled, and are henceforth referred to as “‘Fab’ Fraction”. The Protein A bound fragments were eluted using 100 μL of 0.1 M Glycine, pH 3, by centrifugation at 200×g for 1 min, and immediately neutralized with 10 μL 1M Tris pH 8. Four additional elution steps were performed by centrifugation at 1000×g for 1 min, and neutralized immediately as described. The elution fractions are henceforth referred to as “‘Fc’ fraction”.

SEC-HPLC

Analytical SEC-HPLC was performed on an Agilent 1260 series HPLC system equipped with a degasser (G4225A), binary pump (G1312B), autosampler (G1329B), and diode array detector (DAD, G4212B). Approximately 60 to 100 μg of each untreated antibody, and 100 μL of the “Fab” fraction, and “Fc” fraction were loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 30 min in PBS, pH 7.4. The DAD was set to collect absorbance at 280 nm.

CE-SDS

Capillary gel electrophoresis was performed using the LabChip GXII Touch HT system (PerkinElmer), and samples were analyzed using the ProteinExpress 200 High Sensitivity assay (PerkinElmer, #CLS960008) under reducing and non-reducing conditions. 2 μg of each untreated antibody, and 5 μL of each of the digested pool, “Fab” fraction, and “Fc” fraction were mixed with 7 μL of reducing or non-reducing denaturing solution, and incubated at 70° C. for 10-12 min. The reducing denaturing solution was prepared by adding 24.5 μL of 1 M DTT to 700 μL of non-reducing denaturing solution provided in the kit. Denatured samples were diluted with 32 μL of MilliQ water, mixed well, and spun down prior to analysis. The Protein Express LabChip (PerkinElmer, #760499), and ladder were prepared according to manufacturer instructions.

Results

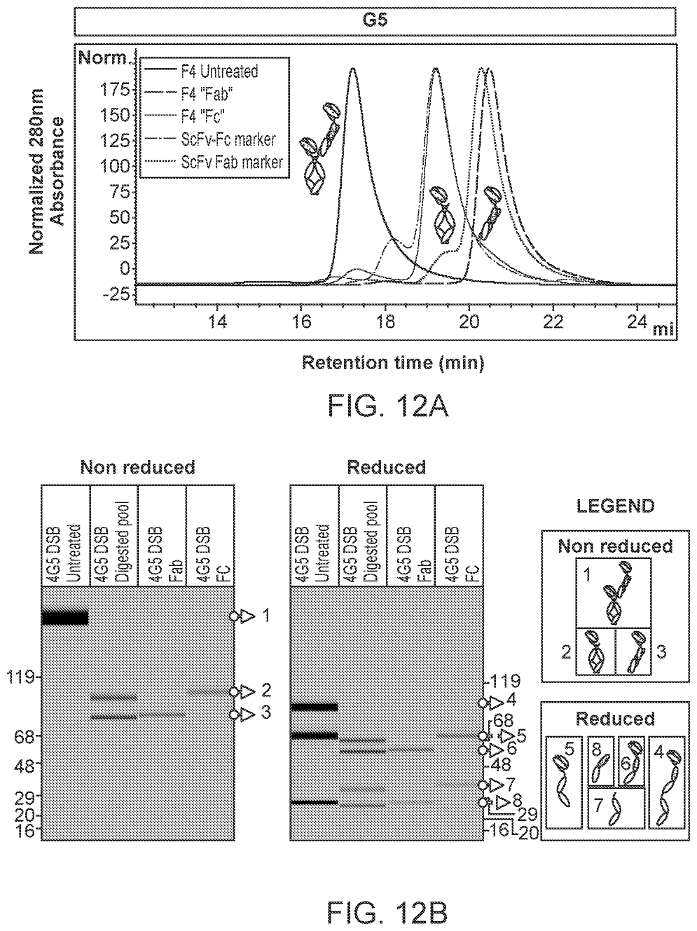

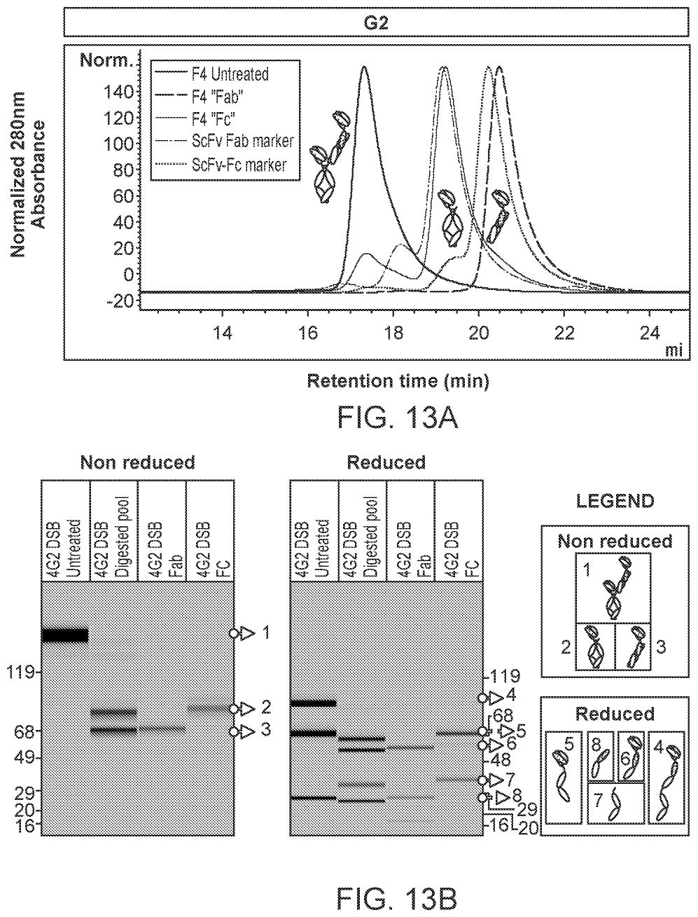

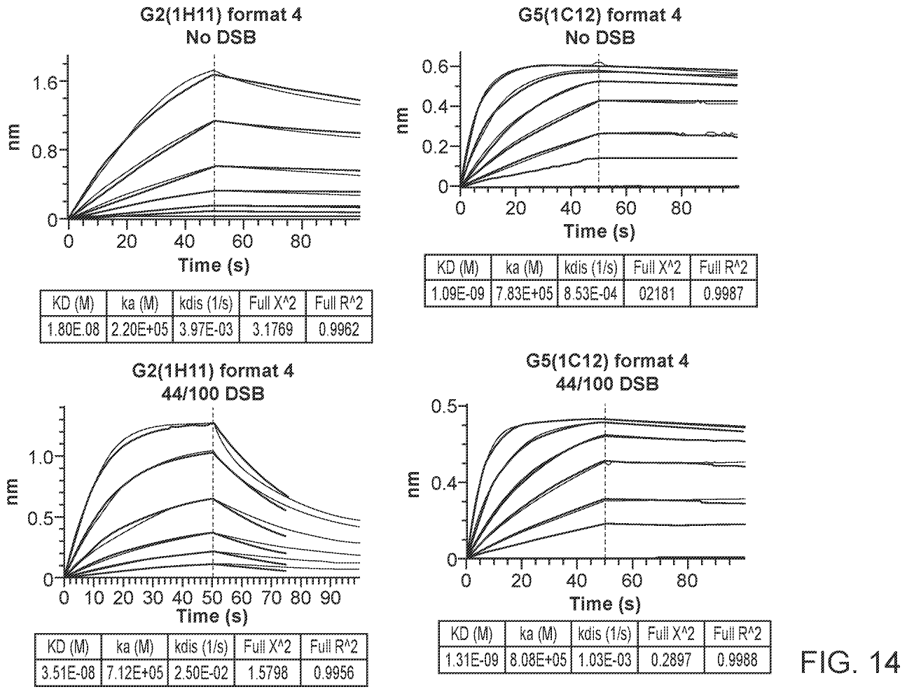

Format 4 molecules stabilized in the 2×ScFv conformation were expected to exhibit a single peak when analyzed by SEC-HPLC. Both undigested G2 and G5 molecules containing the DSB H44/L100 mutation were observed to migrate as single peaks, with a retention time of approximately 17.3 minutes (FIG. 12A and FIG. 13A), suggesting that the putative diabody formation had been eliminated via disulfide bond stabilization. Further, proteolysis of these format 4 molecules resulted in two fragments, namely the ScFv-Fc fragment (#2 in FIG. 12B and FIG. 13B), and the ScFv-Fab fragment (#3 in FIG. 12B and FIG. 13B), as observed in the non-reducing gel. Complete separation of these fragments is observed upon purification of the digested pool by protein A when analyzed by both, CE-SDS and SEC-HPLC. The absence of residual ScFv-Fab related bands in the “Fc” fraction lanes under both reducing and non-reducing conditions indicated stabilization of the 2×scFv conformation for both the G2 and G5 antibodies. Additionally, the absence of a significant peak that aligns with the undigested samples in the Fc-fraction when analyzed by SEC-HPLC also indicated that the 2×ScFv conformation has been successfully stabilized in both G2 (FIG. 13A) and G5 (FIG. 12A) molecules.

### Example 14: Effect of Engineered DSB on Apparent Affinity as Measured by BLI

Format 4 bispecific antibodies with or without DSB mutations as described in Example 12 were generated. The affinity of wildtype and DSB mutants were analyzed on the ForteBio Octet HTX in 96-channel mode with biolayer interferometry (BLI) detection. High Precision Streptavidin SAX biosensors (P/N 18-5117) were loaded into the instrument. Biotinylated G2-pHLA or G5-pHLA was captured on the SAX biosensor at 2 μg/mL and ran for 120 s in the assay buffer composed of 0.02% Tween-20 and 0.1% BSA. The biosensors were then dipped in assay buffer for a baseline. Subsequently, the biosensors were dipped into wells containing varying concentrations of the bispecific antibody samples (3.125, 6.25, 12.5, 25, 50, 100 and 200 nM) to measure the association rate for 50 seconds. The biosensors were finally dipped into wells containing assay buffer to measure the dissociation rate for another 50 seconds. Referencing was completed by having a biosensor with no immobilized ligand dipped into analyte. Kinetic data was processed with Octet™ software using a 1:1 kinetic model with errors within 10%, X² below 3, and R² above 0.9.

Results are depicted in FIG. 14. Introduction of the DSB mutation increased the K_{D} of the G2 Format 4 bispecific from 18 nM to 35.1 nM. Introduction of the DSB mutation increased the K_{D} of the G5 Format 4 bispecific from 1.09 nM to 1.35 nM.

### Example 15: Effect of DSB on Apparent Affinity as Measured by MSD

The effect of the stabilizing DSB on cell binding of Format 4 G2 and G5 antibodies was assessed using the Meso Scale Discovery (MSD) U-PLEX Development Pack, 9-assay (cat. No. K15234N). Biotinylated pHLA and biotinylated Protein A were each diluted to 33 nM using PBS+0.5% BSA. For each plate, 200 μL of the diluted pHLA or protein A was mixed with 300 μL Linker and incubated at room temperature for 30 minutes.

Following the 30 minute incubation, 200 μL Stop solution was added to each linker-pHLA solution. They were again incubated for 30 minutes at room temperature. These volumes were scaled based on the number of plates. At this point, the linker-pHLA solutions were a 10× solution. They were then pooled together and further diluted with stop solution to the final 1× concentration. All volumes were scaled for additional plates. The pooled linker-pHLA solutions were then coated onto the 10-spot plate as 50 μL/well, the plate sealed and stored at 4° C. overnight.

Format 4 G2(1H11) and Format 4 G5(1C12) antibodies, with or without the DSB described in Example 12, were serially diluted 3-fold with PBS+1% BSA. The plate was washed 3 times with PBS+0.05% Tween and samples added as 50 μL/well. Plates were incubated at room temperature shaking for 2 hours. The plates were washed as before and 50 μL of 1 μg/mL SulfoTag donkey anti-human Fc, (Jackson ImmunoResearch 709-005-098) was added to each well. The anti-human Fc antibody was sulfo-tag labeled using the MSD Gold Sulfo-tag NHS-Ester Conjugation kit (Meso Scale Discovery, R31AA-2) at a challenge ratio of 10. The plates were incubated for 1 hour shaking at room temperature. The plate wash was repeated and 150 μL 2× Read Buffer T (Meso Scale Discovery, R92TC-2) was added to all wells and the plate read immediately on the Quickplex SQ 120.

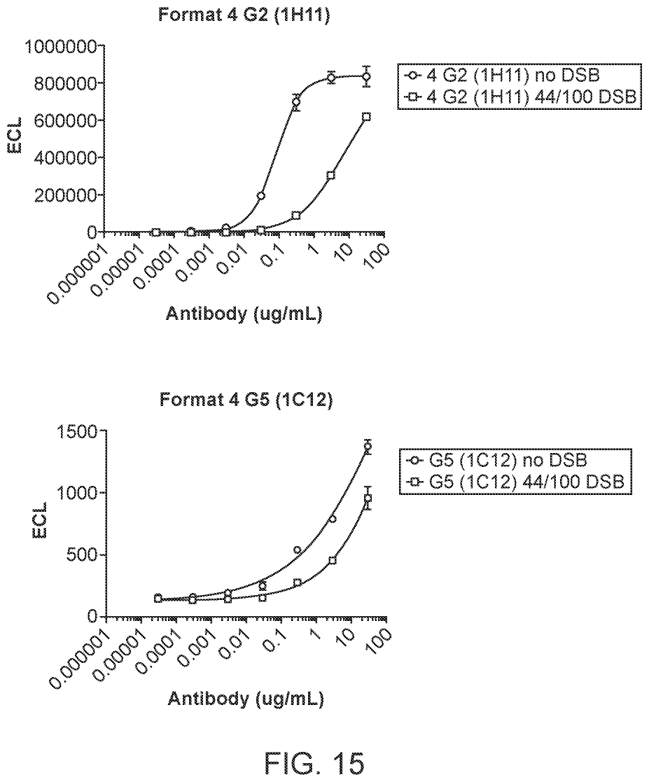

Results are depicted in FIG. 15. G2 Format 4 binding as measured by MSD is 0.546 nM without the DSB and 46.42 nM with the DSB. The G5 data did not fit a curve. However, the G5 dose-response curve without the DSB was leftward shifted as compared to the G5 dose-response curve with the DSB.

### Example 16: Effect of Engineered DSB on Cell Binding

Format 4 bispecific antibodies with and without the stabilizing DSB as described in Example 12 were tested for their ability to specifically bind to the HLA-PEPTIDE targets on the surface of antigen presenting cells.

The cell lines used to express the desired HLA-PEPTIDE targets were as follows: A375 cells (which express HLA subtype *01:01) engineered to express the G2 restricted peptide NTDNNLAVY (SEQ ID NO: 5), LN229 (which express HLA subtype B*35:01) engineered to express the G5 restricted peptide EVDPIGHVY (SEQ ID NO: 9). All cell lines were also engineered to express luciferase.

Tumor cells engineered to express target peptide were harvested, washed in PBS, and stained with eBioscience Fixable Viability Dye eFluor 450 for 15 minutes at room temperature. Following another wash in PBS+1-2% FBS, cells were resuspended with the indicated molecules at varying concentrations and incubated for 1 hour at 4° C. After another wash, PE-conjugated goat anti-human IgG secondary antibody (Jackson ImmunoResearch) was added at 1:100 to 1:200 for 30 minutes at 4° C. After washing in PBS+1-2% FBS, cells were resuspended in PBS+1-2% FBS and analyzed by flow cytometry. Flow cytometric analysis was performed on the Attune NxT Flow Cytometer (ThermoFisher) using the Attune NxT Software. Data was analyzed using FlowJo.

Results

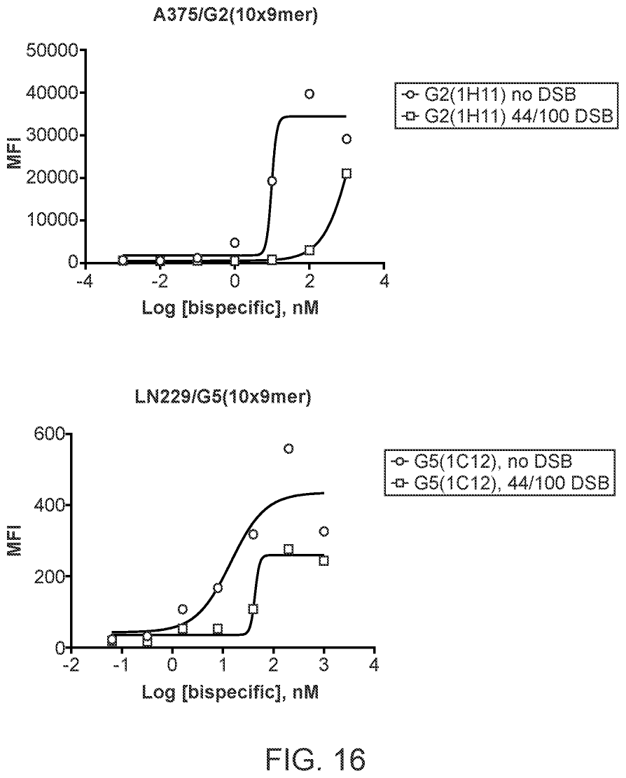

Results are depicted in FIG. 16. Introduction of the stabilizing H44/L100 DSB reduces cell binding for G2(1H11) as measured by an EC50 shift from 9.8 nM without the DSB to 1.75 μM with the DSB. For G5(1C12), addition of the DSB shifted the EC50 from 14.3 nM to 43.2 nM.

### Example 17: In Vitro Cytotoxicity for G2 and G5 Format 4+/−DSB

Materials and Methods

Spheroid Toxicity

The cell lines used to express the desired HLA-PEPTIDE targets were as follows: A375 cells (which express HLA subtype *01:01) engineered to express the G2 restricted peptide NTDNNLAVY (SEQ ID NO: 5), LN229 (which express HLA subtype B*35:01) engineered to express the G5 restricted peptide EVDPIGHVY (SEQ ID NO: 9). All cell lines were also engineered to express luciferase.

Luciferase expressing cells were plated in 100 μL at 10,000-15,000 cells/well in Corning ultra-low attachment plates (Corning #4515) in corresponding culture medium without selection. Plates were incubated for two days at 37° C. and 5% CO2 to allow spheroid formation. Antibody (Format 4 G5(1C12)-hOKT3 or Format 4 G2(1H11), plus or minus the stabilizing disulfide bond described in Example 12), was titrated at and added as 10 μL/well. Normal human PBMCs were thawed and rested for 4-6 hours at 37° C. and added as 100,000 cells/well in 50 μL giving an Effector:Target ratio of 10:1. Plates were then incubated for 4 days at 37° C. and 5% CO2. At the end of the incubation period 100 μL Luciferin (Pierce #88292) at 300 μg/mL was added to the plate. Luciferase was read on the SpectraMax iE3. Percent cytotoxicity was calculated as (Media control-sample signal)/(Media control-maximum lysis)*100.

2D Cytotoxicity

Target and control cells were plated at 40,000 cells per well of 96 well plate. For the G5 molecules the target cell line was LN229 transduced with a 10×9mer cassette expressing the target peptide and luciferase. LN229s transduced with luciferase alone serve as a negative control. For the G2 molecules the target cell line with A375 transduced with a 10×9mer cassette expressing the target peptide and luciferase. A375s transduced with luciferase alone serve as a negative control. After allowing the cells to adhere for 30 minutes, human PBMCs (Stem Cell Technologies) were added at a ratio of 5:1 effector to target cells. Bispecific antibody was added to the well at indicated final concentration. Each concentration was performed in duplicate. Cultures were incubated for three days. Luciferase signal was assessed using Promega's Bio-Glo assay system (Cat. #G7941) according to manufacturer's instructions and read on the SpectraMax M5. Signal was normalized to control wells to determine the percent of cytotoxicity. Loss of luciferase signal is interpreted as loss of cell viability.

Results

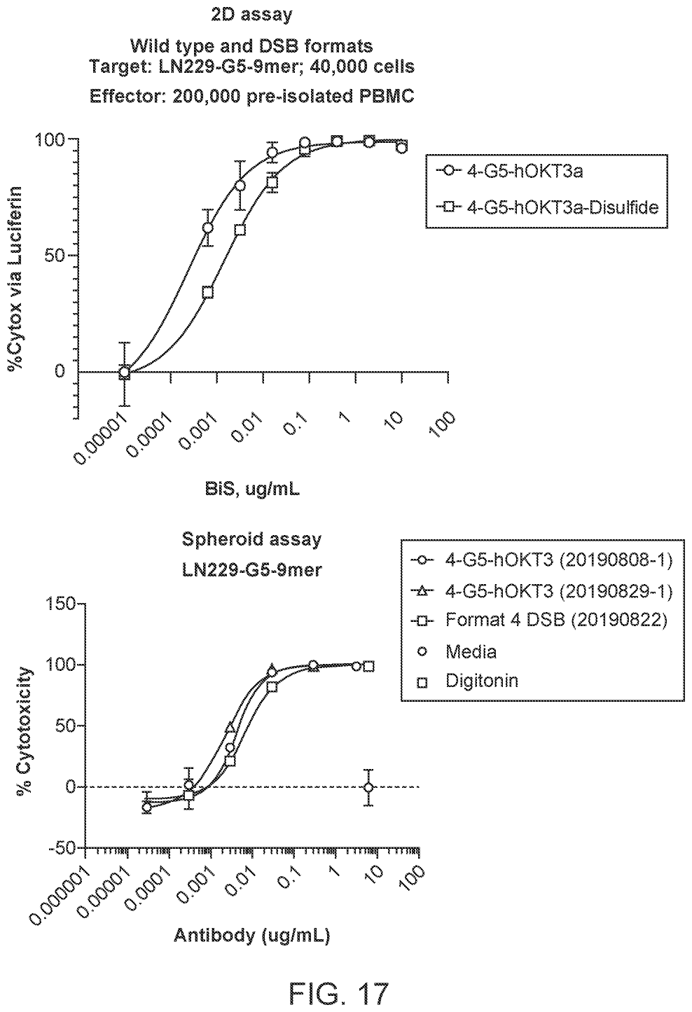

Results for G5 are depicted in FIG. 17. Introduction of the stabilizing disulfide bond resulted in lower cytotoxicity, as indicated by the rightward shift in the dose-response curve.

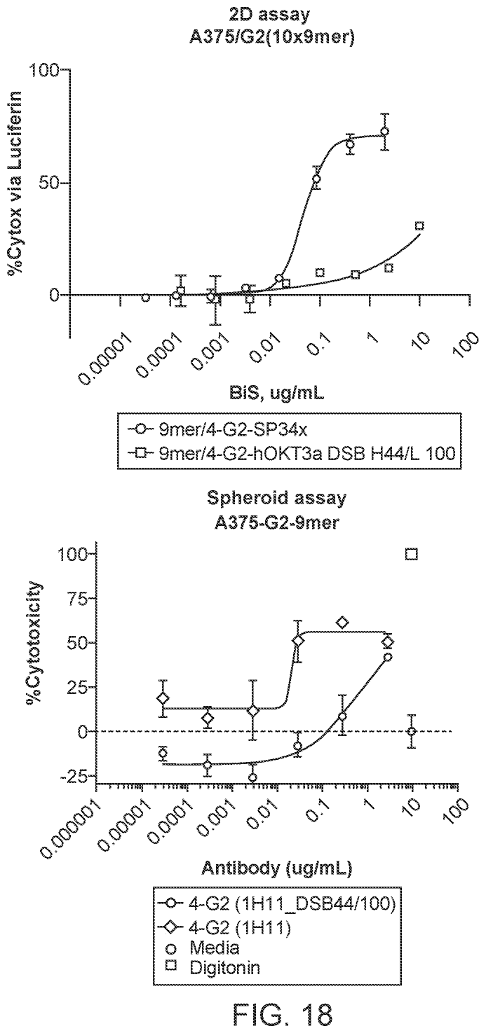

Results for G2 are depicted in FIG. 18. G2 Format 4 antibodies with the stabilizing disulfide bond resulted in lower cytotoxicity, as indicated by the rightward shift in the dose-response curve.

### Example 18: Preparation of Samples for Diabody Experiments

Samples/molecules used in Examples 19-25 and corresponding linker lengths are provided in Table 41 below.

TABLE 41

| | | | | L1 = L2 | L3 = L4 |
|---|---|---|---|---|---|
| | Sample | | | (Linker | (Linker |
| Target | # | Format | Molecule ID | Length) | Length) |
| G5 | 1 | 3 | 3-G5(1C12)-hOKT3a_N97G | | |
| | 2 | 5 | 5-G5(1C12)-hOKT3A DSB | | |
| | | | H44/L100 | | |
| | 3 | 4 | F4 G5(1C12)-hOKT3a | (G4S)x4 (SEQ ID | (G4S)x2 (SEQ ID |
| | | | | NO: 3) | NO: 4) |
| | 4 | 4_DSB | 4-G5-hOKT3a_DSB H44/L100 | (G4S)x4 (SEQ ID | (G4S)x2 (SEQ ID |
| | | | TM(+)cys; N97G; 153R; Y100dV | NO: 3) | NO: 4) |
| | 5 | 4- | 4-G5(1C12)_10AAL hOKT3a | (G4S)x2 (SEQ ID | (G4S)x2 (SEQ ID |
| | | Diabody | | NO: 4) | NO: 4) |
| G2 | 6 | 4 | 4-G2(1H11)-hOKT3a | (G4S)x4 (SEQ ID | (G4S)x2 (SEQ ID |
| | | | | NO: 3) | NO: 4) |
| | 7 | 4-DSB | 4-G2(1H11)-H44/L100 | (G4S)x4 (SEQ ID | (G4S)x2 (SEQ ID |
| | | | hOKT3a-TM | NO: 3) | NO: 4) |
| | 8 | 4- | 4-G2(1H11)-10AAL hOKT3a | (G4S)x2 (SEQ ID | (G4S)x2 (SEQ ID |
| | | Diabody | | NO: 4) | NO: 4) |
| | 9 | 4- | 4-G2(1H11)-10AAL-DSB | (G4S)x2 (SEQ ID | (G4S)x2 (SEQ ID |
| | | Diabody | H44/L100 hOKT3a-TM | NO: 4) | NO: 4) |
| | | X DSB | | | |

TM denotes the TM mutations (L234F/L235E/P331S) in the variant CH2-CH3 domains (both arms of the antibody).

### Example 19: Proteolytic Digestion of Format 3 and Format 5 Antibodies

Materials and Methods

Proteolysis by FabALACTICA

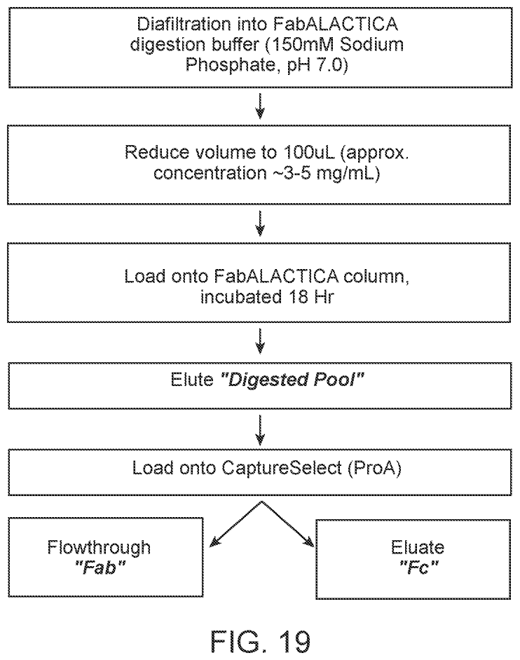

0.3-0.5 mg of G5(1C12) Format 3 and G5(1C12)-DSB H44/L100 Format 5 antibodies were buffer exchanged from PBS pH 7.4 into 150 mM sodium phosphate buffer at pH 7.0. The samples were then concentrated to a volume of approximately 100 μL, with corresponding concentrations ranging from 3-5 mg/mL, loaded onto FabALACTICA microspin columns (Genovis), and incubated for 18 hr with end-over-end mixing. To collect the digested products, the columns were centrifuged at 1000×g for 1 min, followed by three additional rounds of elution using 100 μL PBS pH 7.4. The elution fractions were pooled (referred to as “digested pool”), and subsequently loaded onto a CaptureSelect ProteinA (Genovis) column, and incubated for a minimum of 30 min with end-over-end mixing. The flowthrough was collected by centrifugation at 200×g for 1 min, followed by three wash steps with 100 μL PBS pH 7.4. The flowthrough and wash fractions were pooled, and are henceforth referred to as “‘Fab’ fraction”. The ProteinA bound fragments were eluted using 100 μL of 0.1M Glycine, pH 3 by centrifugation at 200×g for 1 min, and immediately neutralized with 10 μL 1M tris pH 8. Four additional elution steps were performed by centrifugation at 1000×g for 1 min, and neutralized immediately as described. The elution fractions are henceforth referred to as “‘Fc’ Fraction”. The digestion process is outlined in FIG. 19.

SEC-HPLC

Analytical SEC-HPLC was performed on an Agilent 1260 series HPLC system equipped with a degasser (G4225A), binary pump (G1312B), autosampler (G1329B), and diode array detector (DAD, G4212B). Approximately 60 to 100 μg of each untreated antibody, and 100 μL of the “Fab” fraction, and “Fc” fraction were loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 30 min in PBS, pH 7.4. The DAD was set to collect absorbance at 280 nm.

CE-SDS

Capillary gel electrophoresis was performed using the LabChip GXII Touch HT system (PerkinElmer), and samples were analyzed using the ProteinExpress 200 High Sensitivity assay (PerkinElmer, #CLS960008) under reducing and non-reducing conditions. 2 μg of each untreated antibody, and 5 μL of each of digested pool, digested “Fab” fraction, and digested “Fc” fraction were mixed with 7 μL of reducing or non-reducing denaturing solution, and incubated at 70° C. for 10-12 min. The reducing denaturing solution was prepared by adding 24.5 μL of 1M DTT to 700 μL of non-reducing denaturing solution provided in the kit. Denatured samples were diluted with 32 μL of MilliQ water, mixed well, and spun down prior to analysis. The Protein Express LabChip (PerkinElmer, #760499), and ladder were prepared according to manufacturer instructions.

Results

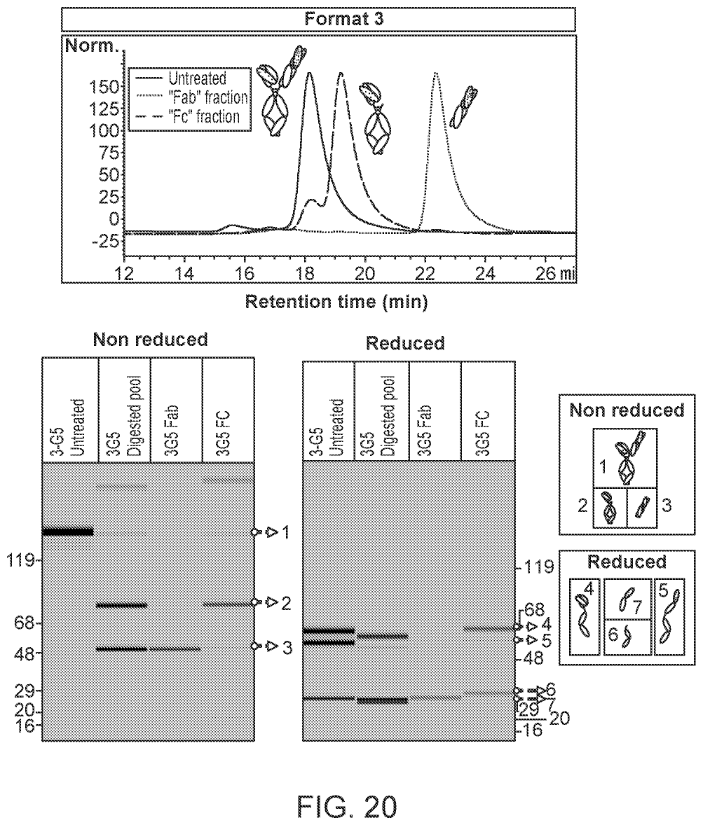

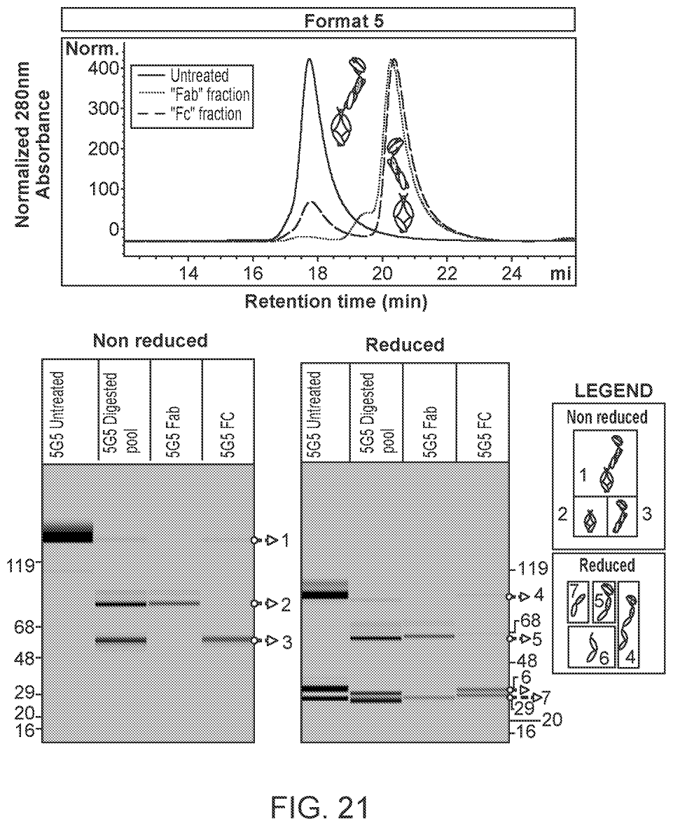

Format 3 and Format 5 antibodies were digested to create fragment markers for subsequent analysis of variants of G2 and G5 format 4 antibodies by SEC-HPLC. Proteolysis of Format 4 antibodies using the FabALACTICA enzyme were expected to result in an ScFv-Fc fragment and an ScFv-Fab fragment, as Explained in Examples 9 and 10. Upon proteolysis of format 3 antibody and purification by ProteinA, the “Fe fraction”, yielded the ˜75 kDa ScFv-Fc standard, which had a retention time around 19.5 minutes when analyzed by SEC-HPLC (FIG. 20). Similarly, digestion of the format 5 antibody, and collection of the Protein A flowthrough, the “Fab” fraction yielded the ˜75 kDa ScFv-Fab standard, which had a retention time of around 20.5 minutes when analyzed by SEC-HPLC (FIG. 21). The one minute difference in retention times observed between the two fragment markers was sufficient to reliably analyze assembly of format 4 variants by proteolysis.

### Example 20: Effect of Shortened Linker on Diabody Formation

Materials and Methods

Proteolysis by FabALACTICA

0.3-0.5 mg of G5(1C12)-hOKT3a and G2(1H11)-hOKT3a Format 4 antibodies with and without shortened linkers in L1 and L2 were buffer exchanged from PBS pH 7.4 into 150 mM sodium phosphate buffer at pH 7.0. Format 4 antibodies with shortened L1 and L2 linkers had 10 amino acid residues at each of the L1 and L2 linkers, specifically (GGGGS)₂ (SEQ ID NO: 4), The samples were then concentrated to a volume of approximately 100 μL, with corresponding concentrations ranging from 35 mg/mL, loaded onto FabALACTICA microspin columns (Genovis), and incubated for 16 hr with end-over-end mixing. To collect the digested products, the columns were centrifuged at 1000×g for 1 min, followed by two additional rounds of elution using 100 μL PBS pH 7.4. The elution fractions were pooled (referred to as “digested pool”), and subsequently loaded onto a CaptureSelect ProteinA (Genovis) column, and incubated for a minimum of 30 min with end-over-end mixing. The flowthrough was collected by centrifugation at 200×g for 1 min, followed by two wash steps with 100 μL PBS pH 7.4. The flowthrough and wash fractions were pooled, and are henceforth referred to as “‘Fab’ fraction”. The ProteinA bound fragments were eluted using 100 μL of 0.1 M Glycine, pH 3 by centrifugation at 200×g for 1 min, and immediately neutralized with 10 μL 1M tris pH 8. A second elution step was performed by centrifugation at 1000×g for 1 min, and neutralized immediately as described. The elution fractions are henceforth referred to as “‘FC’ Fraction”

SEC-HPLC

For the G5(1C12) Format 4 molecule with L1=L2=(G4S)×4 (SEQ ID NO: 3), analytical SEC-HPLC was performed on an Agilent 1200 series HPLC system equipped with a degasser (G1379B), binary pump (G1312B), high performance autosampler (G1367D), and wide range diode array detector (DAD, G7115A). Approximately 40 μL of each of the untreated antibody, “Fab” Fraction, and “Fc” fraction was loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 60 min in PBS, pH 7.4. The DAD was set to collect absorbance at 280 nm.

For all other molecules, analytical SEC-HPLC was performed on an Agilent 1260 series HPLC system equipped with a degasser (G4225A), binary pump (G1312B), autosampler (G1329B), and diode array detector (DAD, G4212B). Approximately 60 to 100 μg of each untreated antibody, and 100 μL of the “Fab” fraction, and “Fc” fraction were loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 30 min in PBS, pH 7.4. The DAD was set to collect absorbance at 280 nm.

SDS-PAGE

For all molecules tested, 2 μg of undigested antibody, and 5-10 μL of digested pool, “Fab” fraction, and “Fc” fraction were denatured under non-reducing and reducing conditions using NuPage 4×LDS sample buffer (Invitrogen). Samples analyzed under non-reducing conditions were left at ambient temperature. Samples analyzed under reducing conditions, using 2 μL of 1M DTT, were incubated at 70° C. for 5-10 minutes. All samples were analyzed using NuPage 4-12% Bis-Tris gradient gels (Invitrogen) against Precision Plus Protein™ Dual Color Standards (Bio-Rad), with NuPage MOPS SDS running buffer (Invitrogen). Gels were visualized after staining with InstantBlue stain (Expedeon).

Results

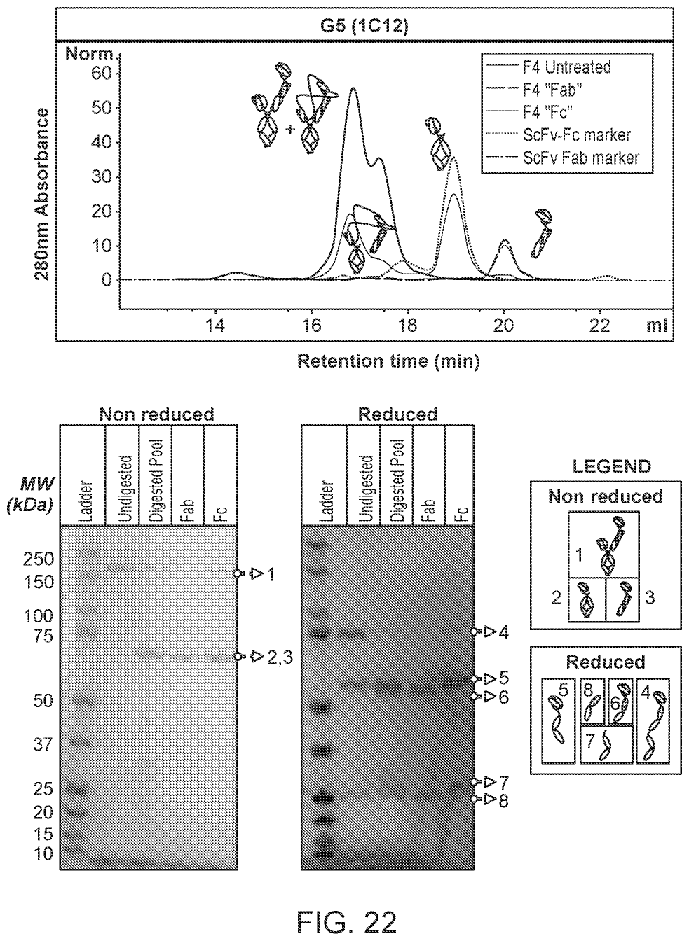

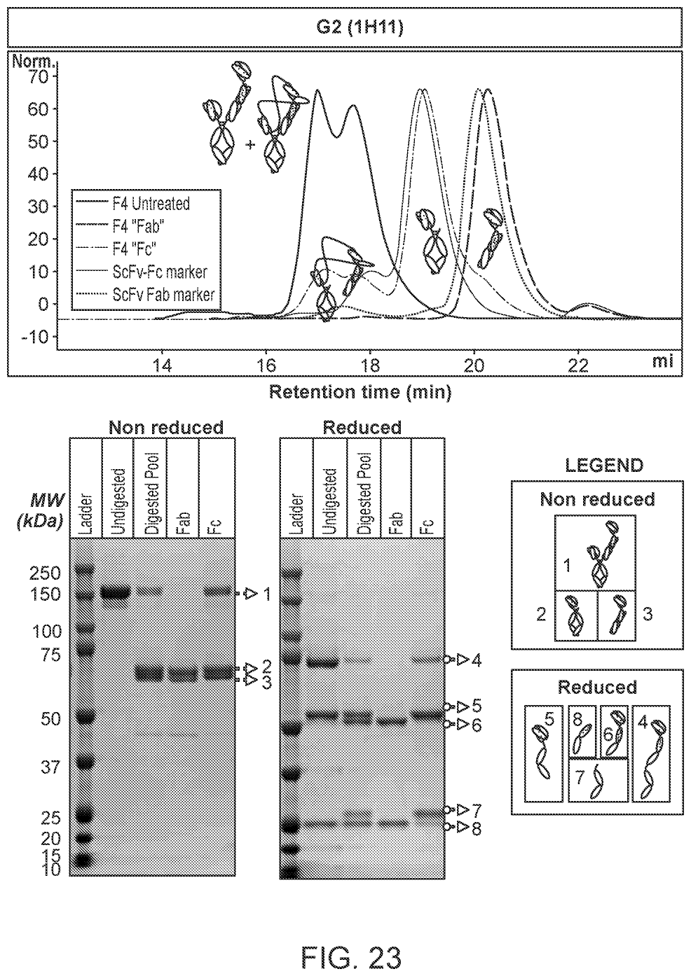

SEC-HPLC analysis of undigested format 4 G2(1H11) and G5(1C12) molecules with L1=L2=(G4S)×4 (SEQ ID NO: 3) showed the characteristic “split-peak” profile described previously (FIG. 22 and FIG. 23), where the peak with retention time around 17.3 minutes corresponds to the 2×ScFv conformation, and the peak with retention time around 17.7 minutes corresponds to the putative diabody conformation. This “split-peak” was more pronounced for the G2(1H11) molecule, indicating that the ScFv of this molecule undergoes more “breathing”, resulting in more frequent shuffling between the two conformations. SEC-HPLC analysis of the “Fab” fraction for both molecules revealed a single peak that aligns with the retention time of ScFv-Fab marker. The “Fc” fractions had a peak with retention time that aligned with the ScFv-Fc marker, as well as a second peak with retention time that aligned with the undigested antibody. The absence of an intense band migrating around 150 kDa under non-reducing conditions, and around 75 kDa under reducing conditions in the “Fc” fraction lane for the G5 molecule indicated that the digestion was near complete. Yet, the second peak on the SEC-HPLC chromatogram that aligned with the retention time of the undigested antibody appeared in roughly equal proportion to the ScFv-Fc peak. The presence of this second peak, which likely corresponds to a clipped diabody, further indicated that the G5 Format 4 molecule with a 20 amino acid-long linker at locations L1 and L2 exists as a mixture of the 2×ScFv and diabody conformations. The observation that the second peak for the G2(1H11) molecule in the “Fc” fraction was not as significant as was observed for G5(1C12) is likely explained by more frequent shuffling between the diabody and 2×ScFv conformations mentioned supra. During the 16 hr digestion period, the clipped diabody form can more readily dissociate into the ScFv-Fc and ScFv-Fab fragments, contributing to the results obtained for this molecule (FIG. 22 and FIG. 23).

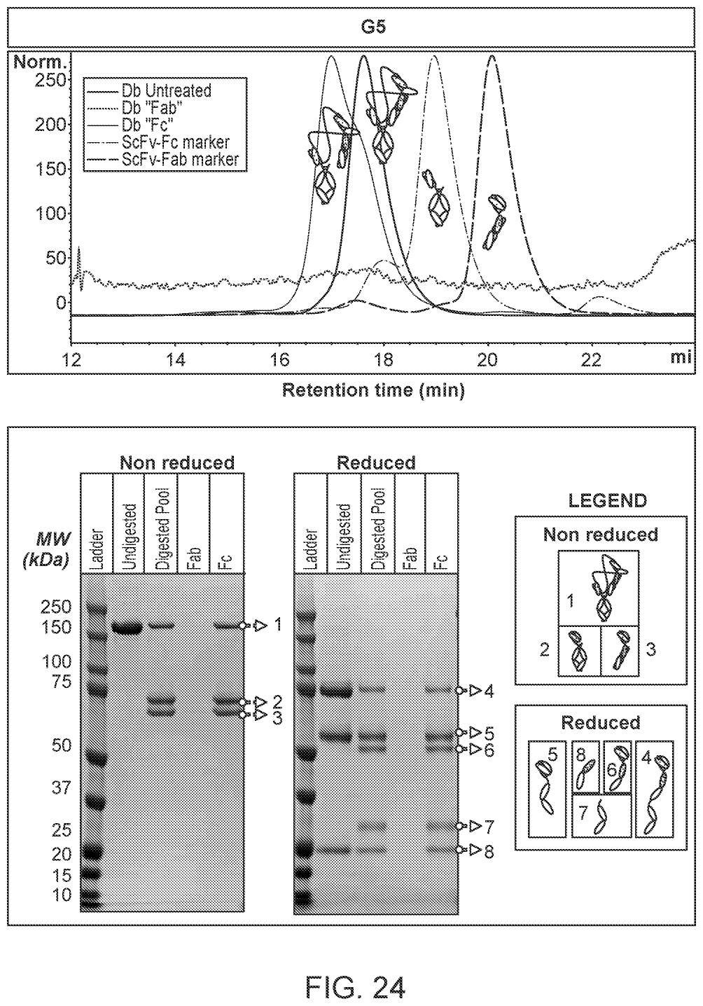

On the other hand, SEC-HPLC analysis of undigested format 4 G2(1H11) and G5(1C12) molecules with shortened L1 and L2 linkers (L1=L2=(G4S)×2 (SEQ ID NO: 4)) showed a single peak with retention time around 17.7-18 minutes (FIG. 24 and FIG. 25). This retention time aligns with what we hypothesized to be the peak corresponding to the diabody conformation in the split-peak profile observed prior to shortening the linker. Therefore, this indicated that shortening the linker from 20 amino acids to 10 amino acids forced both format 4 molecules into the diabody conformation.

To further support this, no protein was recovered in the G5(1C12) “Fab” fraction, as shown in the SEC-HPLC chromatogram, and reducing and non-reducing gels. Additionally, the “Fc” fraction that resulted from digestion of this molecule resulted in a single peak on the SEC-HPLC chromatogram which aligned with the retention time of the undigested molecule, corresponding to a clipped diabody. The newly formed “split peak” in the Fc-fraction is likely due to the clipped diabody existing in compact and extended conformations. Furthermore, the reducing and non-reducing gels showed that any bands corresponding the ScFv-Fab fragment, which would be expected to be present in the “Fab” fraction lane in the absence of diabody, were present in the ScFv-Fc fraction instead (FIG. 24).

Similar analysis of the “Fc” fraction for the G2(1H11) molecule supported diabody formation, where the SEC-HPLC chromatogram showed a “split peak” corresponding to the clipped diabody conformation, and bands corresponding to ScFc-Fab fragment present in the “Fc” lane of the reducing and non-reducing gel. The “Fab” fraction of the G2(1H11) molecule, however did contain some residual ScFv-Fab, which likely dimerized into a diabody, as the SEC-HPLC trace for this fraction had an earlier retention time than would be expected for the ScFv-Fab fragment (FIG. 25). This is likely a result of the G2(1H11) ScFv being more prone to “breathing”, as mentioned above, where the clipped diabody can readily fall apart into the ScFv-Fc and ScFv-Fab fractions. The increased “breathing” observed for the G2(1H11) molecule indicated that the G2(1H11) molecule with shortened linker might not form the diabody conformation as stably as the G5(1C12) molecule.

### Example 21: Modifications to Stabilize Conformation of Format 4 Antibodies that Specifically Bind G2 Target and CD3

The purpose of this experiment was to identify exemplary modifications to the Format 4 antibody that would stabilize the antibody in one conformation when in solution. Groups of format 4 antibodies targeting HLA-Peptide Target A*01:01_NTDNNLAVY (SEQ ID NO: 5) (G2 target) and CD3 were generated, each with one type of potential modification for Format-4 stability, as shown in FIG. 6C. Examples of the groups of antibodies tested are shown in Table 52. The antibodies were evaluated for stability using size exclusion chromatography (SEC).

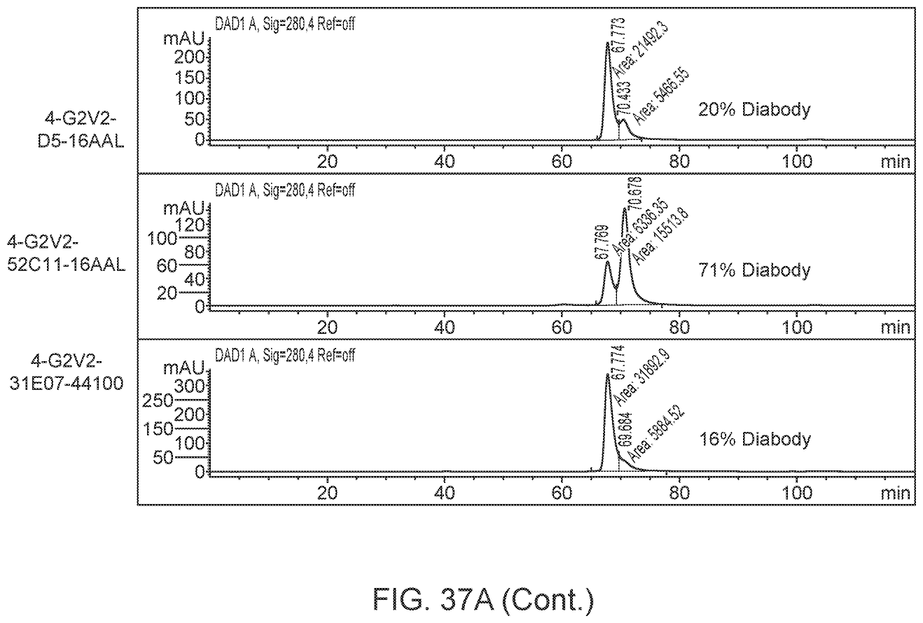

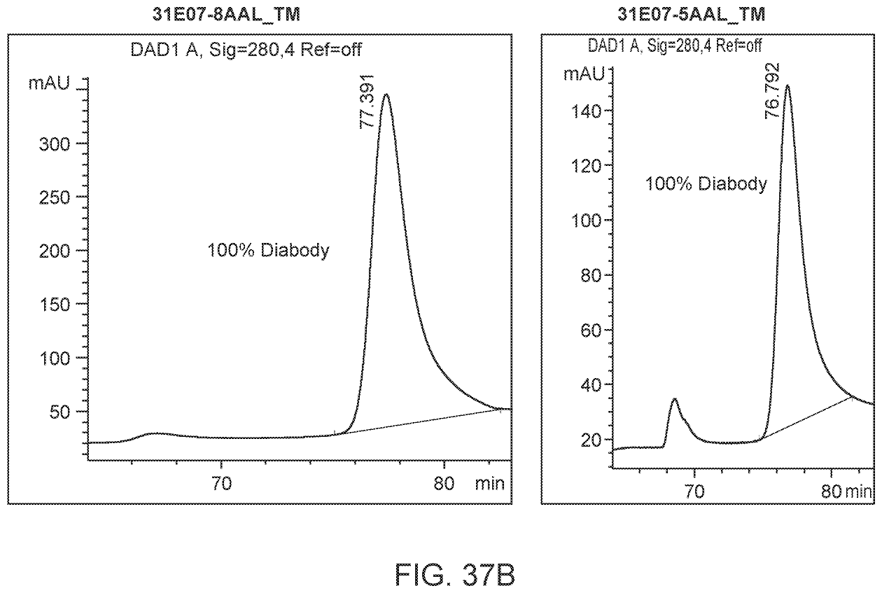

TABLE 52
Modifications tested for stability in Format-4 antibodies. *aa = amino acids

| Format 4 Antibody | |
|---|---|
| (see FIGS. 37A-37B) | Modification for Stability |
| 4-G2V2-C11-16AAL | Shortened linkers; L1 = L2 = 16 aa* |
| 4-G2V2-52C11-Short | Shortened L3 and L4; L3 = GSGGGG-CP (SEQ ID NO |
| | 731) and L4 = GSGGGG (SEQ ID NO: 402) |
| 4-G2V2-52C11-10AAL | Shortened linkers; L1 = L2 = 10 aa |
| 4-G2V2-31E07-10AAL | Shortened linkers; L1 = L2 = 10 aa |
| 4-G2V2-D5-16AAL | Shortened linkers; L1 = L2 = 16 aa |
| 4-G2V2-52C11-16AAL | Shortened linkers; L1 = L2 = 16 aa |
| 4-G2V2-31E07-44100 | Disulfide bond; cysteines at position 44 in the VH |
| | and position 100 in the VL (Kabat) |
| 31E07-8AAL_TM | Shortened linkers; L1 = L2 = 8 aa |
| 31E07-5AAL_TM | Shortened linkers; L1 = L2 = 5 aa |

### Size Exclusion Chromatography

Purified bispecific antibody samples (50 ug) were filtered using 0.22 μm centrifugal filters (VWR P/N 82031-348) and spun at 4000 g for 1 minute to remove any large particulates. Approximately 40 μg of the filtered samples were then loaded on the Agilent HPLC-SEC with TSKgel G3000SWxl column (Tosoh P/N 08541) with TSK guard column (Tosoh P/N 08543). An isocratic method using PBS with Calcium and Magnesium (Corning P/N 21-030-CM) as mobile phase was ran over 120 minutes with a flow rate of 0.125 ml/min to ensure good separation of 2×ScFv and diabody species. Responses were detected and recorded using A280 wavelength. The peaks on the chromatogram were manually integrated and the percentage of the diabody species were calculated based on the total peak areas from each sample.

The SEC chromatograms showed that most of the antibody groups had mixed populations of proteins in solution (i.e. unstable with both the dual scFv and diabody conformations present). However, four groups demonstrated a stable conformation: 4-G2V2-52-C11-10AAL, 4-G2V2-31-E07-10AAL, 31-E07-8AAL_TM, and 31-E07-5AAL_TM. These four antibody groups had shortened 5 aa (5 amino acid), 8 aa or 10 aa linkers present at positions L1 and L2. The four resulting chromatograms showed a single peak resulting from each group, indicating 100% diabody formation (see FIGS. 37A & 37B). The length and compositions of the ABR-region linkers for the 4-G2V2-52-C11-10AAL and 4-G2V2-31-E07-10AAL are shown in Table 53.

TABLE 53
Linker compositions for the 4-G2V2-52-C11-10AAL
and 4-G2V2-31-E07-10AAL

| | ABR-region Linkers |
|---|---|
| L1 | (GGGGS)x2 (SEQ ID NO: 4) |
| L2 | (GGGGS)x2 (SEQ ID NO: 4) |
| Knob (L3) | GGGGS-EPKSSDKTHTCP (SEQ ID NO: 403) |
| Hole (L4) | GGGGSGGGGS (SEQ ID NO: 107) |

Fabalactica Digestion to Confirm Diabody Formation

The results showed that shorter amino acid linker length (e.g., 10 amino acids of (G4S)×2) is enough to form the diabody confirmation for affinity mature molecules. The format 4 antibodies having a linker length of 10 amino acids without DSB were named “Format 41” antibodies. Their formation of diabody was confirmed by FabALACTICA digestion experiment, using the proteolysis by FabALACTICA methods described herein.

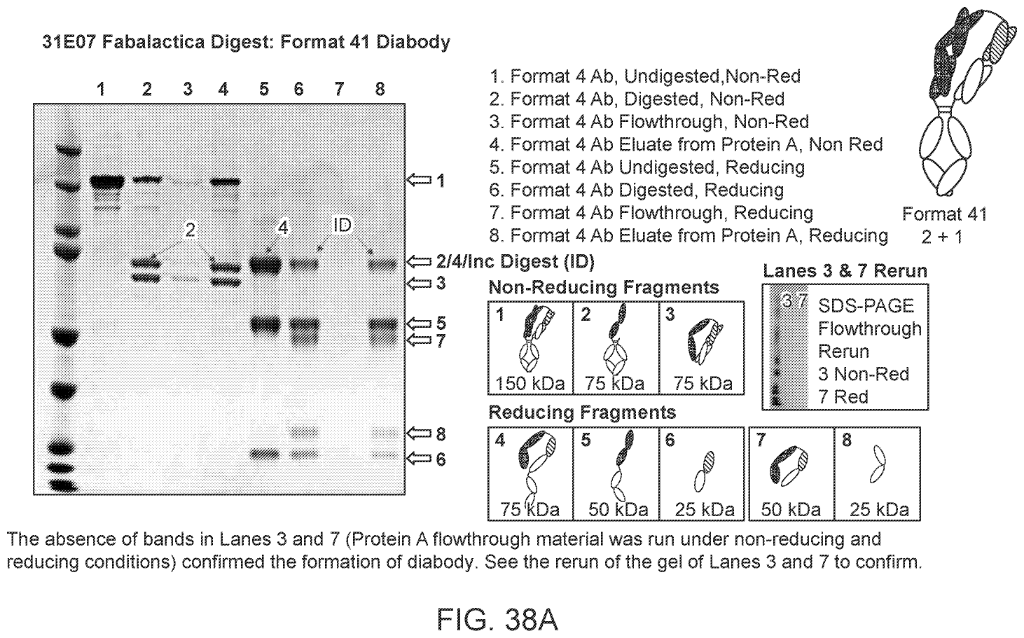

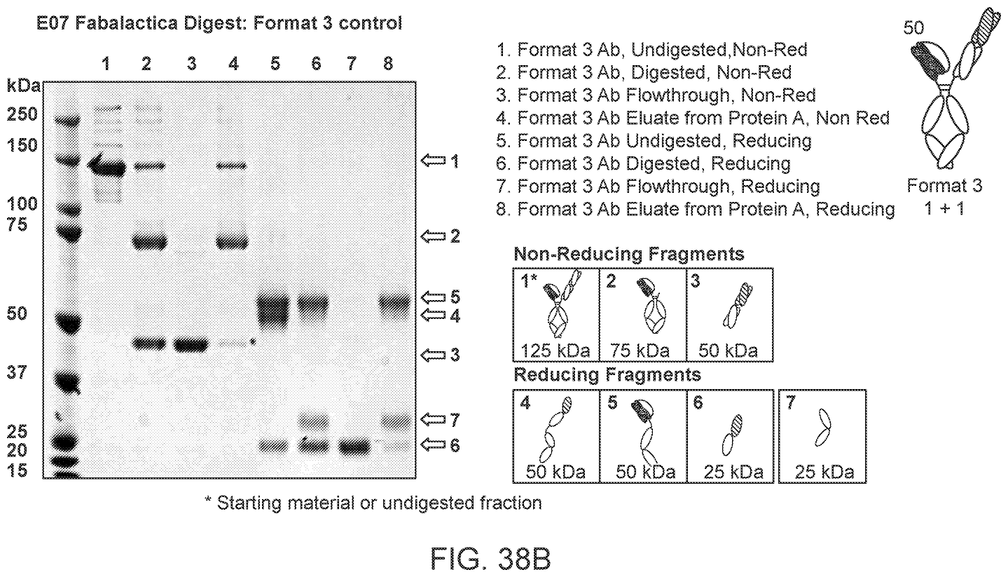

As shown in FIGS. 38A and 38B, the Format 41 antibody formed a diabody. After fabalactica digestion of format 41, whole content was passed through protein A column. The diabody formed by non-covalent interactions between VH(knob)-VL(hole) and VH(hole)-VL(Knob) resulted in retaining of digested fragments in the protein A column. A Format 3 antibody was used as a control. In case of the Format 3 antibody, after FabALACTICA digestion, the Fab portion of the molecule flows through protein A column showing the corresponding band in the SDS-PAGE gel. This can be seen by comparing lanes 3 and 7 of the FIG. 38A (format 41) and FIG. 38B (format 3).

### Example 22: Effect of Combining DSB with Shortened Linker

Materials and Methods

Proteolysis by FabALACTICA:

0.3-0.5 mg of G2(1H11)-hOKT3a Format 4 with L1=L2=(G4S)x2 (SEQ ID NO: 4) and DSB H44/L100 mutation, was buffer exchanged from PBS pH 7.4 into 150 mM sodium phosphate buffer at pH 7.0. The samples were then concentrated to a volume of approximately 100 μL, with corresponding concentrations ranging from 35 mg/mL, loaded onto FabALACTICA microspin columns (Genovis), and incubated for 18 hr with end-over-end mixing. To collect the digested products, the columns were centrifuged at 1000×g for 1 min, followed by three additional rounds of elution using 100 μL PBS pH 7.4. The elution fractions were pooled (referred to as “digested pool”), and subsequently loaded onto a CaptureSelect ProteinA (Genovis) column, and incubated for a minimum of 30 min with end-over-end mixing. The flowthrough was collected by centrifugation at 200×g for 1 min, followed by three wash steps with 100 μL PBS pH 7.4. The flowthrough and wash fractions were pooled, and are henceforth referred to as “‘Fab’ fraction”. The ProteinA bound fragments were eluted using 10 μL of 0.1 M Glycine, pH 3 by centrifugation at 200×g for 1 min, and immediately neutralized with 10 μL 1 M Tris pH 8. Four additional elution steps were performed by centrifugation at 1000×g for 1 min, and neutralized immediately as described. The elution fractions are henceforth referred to as “‘Fc’ Fraction”

SEC-HPLC

Analytical SEC-HPLC was performed on an Agilent 1260 series HPLC system equipped with a degasser (G4225A), binary pump (G1312B), autosampler (G1329B), and diode array detector (DAD, G4212B). Approximately 60 μg to 100 μg of each untreated antibody, and 100 μL of the “Fab” fraction, and “Fc” fraction were loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 30 min in PBS, pH 7.4. The DAD was set to collect absorbance at 280 nm.

CE-SDS

Capillary gel electrophoresis was performed using the LabChip GXII Touch HT system (PerkinElmer), and samples were analyzed using the ProteinExpress 200 High Sensitivity assay (PerkinElmer, #CLS960008) under reducing and non-reducing conditions. 2 μg of each untreated antibody, and 5 μL of each of digested pool, digested “Fab” fraction, and digested “Fc” fraction were mixed with 7 μL of reducing or non-reducing denaturing solution, and incubated at 70° C. for 10-12 min. The reducing denaturing solution was prepared by adding 24.5 μL of 1M DTT to 700 μL of non-reducing denaturing solution provided in the kit. Denatured samples were diluted with 32 μL of MilliQ water, mixed well, and spun down prior to analysis. The Protein Express LabChip (PerkinElmer, #760499), and ladder were prepared according to manufacturer instructions.

Results

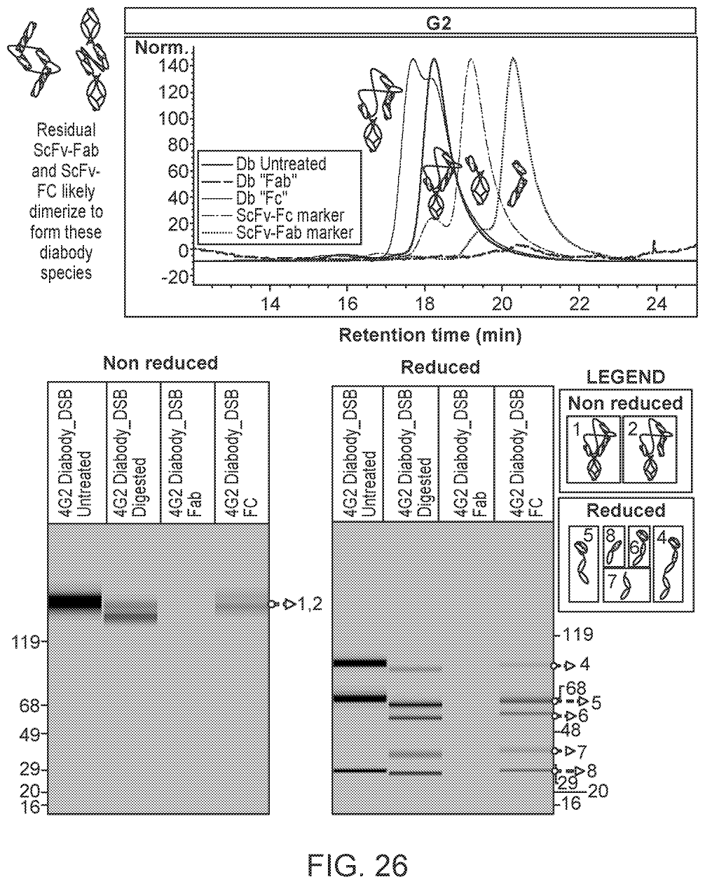

SEC-HPLC analysis of undigested format 4 G2(1H11) shortened L1 and L2 linkers (L1=L2=(G4S)×2) (SEQ ID NO: 4), along with the incorporation of the DSB H44/L100 mutation showed a single peak with retention time around 17.7-18 minutes (FIG. 26). This retention time aligned with what was hypothesized to be the peak corresponding to the diabody conformation in the split-peak profile observed prior to shortening the linker. Contrary to what was seen in the absence of the DSB H44/L100 stabilizing mutation (Example 20), no protein was recovered in the “Fab” fraction, as was observed in the SEC-HPLC chromatogram, and reducing and non-reducing gels. Absence of bands corresponding to ScFv-Fc and ScFv-Fab fragments following digestion under non-reducing conditions indicated that the disulfide bond formation was complete, and was effective at stabilizing the diabody conformation and preventing “breathing” (FIG. 26).

Additionally, the “Fc” fraction that resulted from digestion of this molecule resulted in a single peak on the SEC-HPLC chromatogram which aligned with the retention time of the undigested molecule, corresponding to a clipped diabody. The newly formed “split peak” in this “Fc” fraction was likely due to the clipped diabody existing in compact and extended conformations. Furthermore, the reducing gel showed that any bands corresponding to the ScFv-Fab fragment, which would be expected in the “Fab” fraction lane in the absence of diabody, was instead present only in the ScFv-Fc fraction (FIG. 26). These results indicate that shortening the linker and introduction of a disulfide bond together force and stabilize diabody conformation for the G2(1H11) format 4 molecule.

### Example 23: Effect of Diabody on Affinity as Measured by BLI

Materials and Methods

The affinity of 2×ScFv-conformed ABPs and diabody-conformed ABPs (i.e. G5(1C12) and G2(1H11) Format 4 antibodies with and without shortened linkers L1 and L2) was evaluated using the ForteBio Octet HTX in 96-channel mode with biolayer interferometry (BLI) detection. The experiment utilized G2(1H11)-hOKT3a and G5(1C12) format 4 antibodies with shortened linkers (L1=L2=(GGGGS)₂ (SEQ ID NO: 4), “DAB”) and with non-shortened linkers (L1=L2=(GGGGS)₄ (SEQ ID NO: 3), “2×scFv”). High Precision Streptavidin SAX biosensors (P/N 18-5117) were loaded into the instrument. Biotinylated G2-pHLA or G5-pHLA was captured on the SAX biosensor at 2 μg/mL and ran for 120 s in the assay buffer composed of 0.02% Tween-20 and 0.1% BSA. The biosensors were then dipped in assay buffer for a baseline. Subsequently, the biosensors were dipped into wells containing varying concentrations of the bispecific antibody samples (3.125, 6.25, 12.5, 25, 50, 100 and 200 nM) to measure the association rate for 50 seconds. The biosensors were finally dipped into wells containing assay buffer to measure the dissociation rate for another 50 seconds. Referencing was completed by having a biosensor with no immobilized ligand dipped into analyte. Kinetic data was processed with Octet™ software using a 1:1 kinetic model with errors within 10%, X² below 3, and R² above 0.9.

### Results

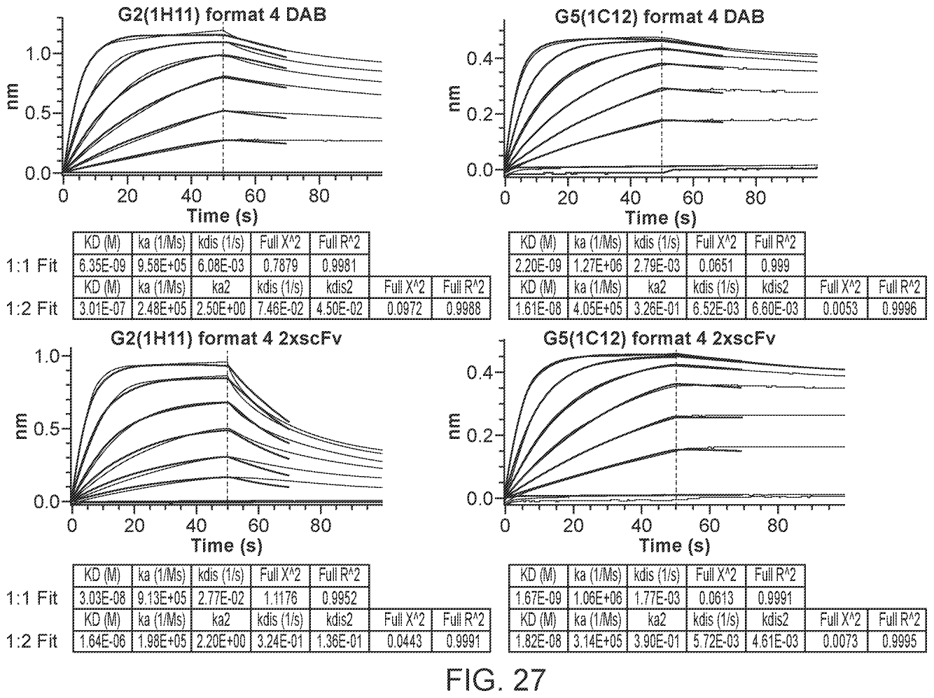

Results are shown in FIG. 27. The results revealed a significantly lower K_{D} value for G2(1H11) format 4 antibody with shortened linker than with the non-shortened linker.

### Example 24: Effect of Diabody Formation on Cell Binding

For G2 cell binding, the experiment utilized G2(1H11)-hOKT3a Format 4 antibody having L1=L2=(GGGGS)₄ (SEQ ID NO: 3) with/or without the DSB44/100. It also utilized a G2(1H11)-hOKT3a Format 4 antibody with the shortened linker (10AAL=L1=L2=(GGGGS)₂) (SEQ ID NO: 4). For the G5 cell binding, the experiment utilized G5(1C12)-hOKT3a Format 4 antibody having L1=L2=(GGGGS)₄ (SEQ ID NO: 3) with/or without the DSB44/100. It also utilized a G5(1C12)-hOKT3a Format 4 antibody with the shortened linker (10AAL=L1=L2=(GGGGS)₂) (SEQ ID NO: 4). The cell lines used to express the desired HLA-PEPTIDE targets were as follows: A375 cells (which express HLA subtype *01:01) engineered to express the G2 restricted peptide NTDNNLAVY (SEQ ID NO: 5), LN229 (which express HLA subtype B*35:01) engineered to express the G5 restricted peptide EVDPIGHVY (SEQ ID NO: 9). All cell lines were also engineered to express luciferase, using a lentivirus transduction of a cassette containing a 10× repeat of the peptide, Luciferase, and puromycin-resistance. Cassette-expressing cells were selected using 0.5 μg/mL of puromycin. Jurkat E6-1 (ATCC TIB-152) and Jurkat T3.5 (ATCC TIB-153) cells were grown under standard tissue culture conditions. Cells were harvested, washed in PBS, and stained with eBioscience Fixable Viability Dye eFluor 450 for 15 minutes at room temperature. Following another wash in PBS+2% FBS, cells were resuspended with bispecifics at varying concentrations. Cells were incubated with bispecifics for 1 hour at 4° C. After another wash, PE-conjugated goat anti-human IgG secondary antibody (Jackson ImmunoResearch) was added at 1:100. After incubating at 4° C. for 45 minutes and washing in PBS+2% FBS, cells were resuspended in PBS+2% FBS and analyzed by flow cytometry. Flow cytometric analysis was performed on the Attune NxT Flow Cytometer (ThermoFisher) using the Attune NxT Software. Data was analyzed using FlowJo.

Results

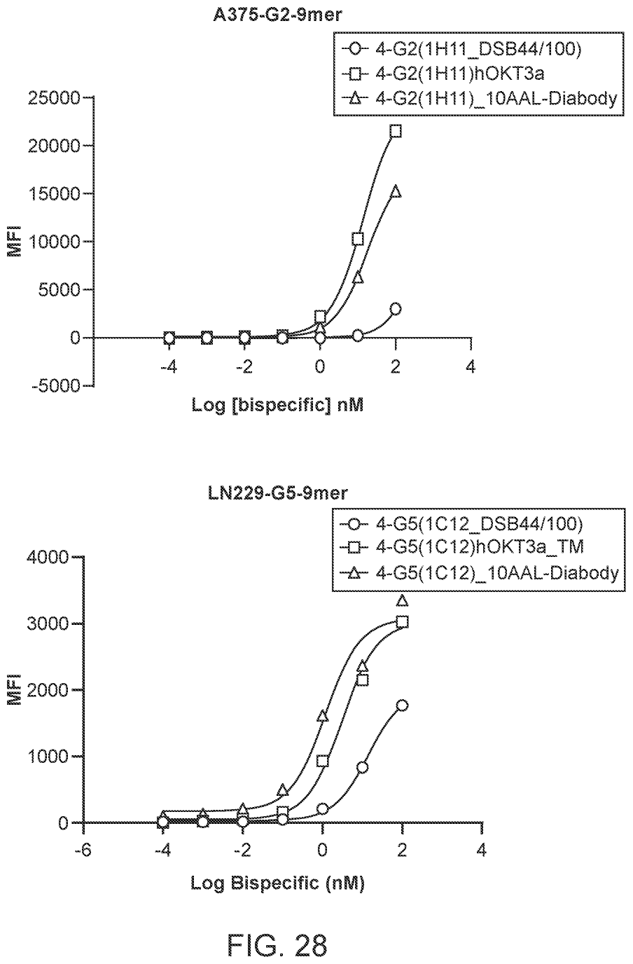

Results are shown in FIG. 28. All the G2 and G5 molecule bound their pHLA targets. For both G2 and G5 groups, the cell binding to molecule with the shortened linker (diabody conformed) exhibited higher levels of cell binding than the non-shortened linker+DSB44/100 groups (FIG. 28).

### Example 25: Effect of Diabody Formation on Cytotoxicity

For G2 cell binding, the experiment utilized G2(1H11)-hOKT3a Format 4 antibody having L1=L2=(GGGGS)₄ (SEQ ID NO: 3) with/or without the DSB44/100. It also utilized a G2(1H11)-hOKT3a Format 4 antibody with the shortened linker (10AAL=L1=L2=(GGGGS)₂) (SEQ ID NO: 4). For the G5 cell binding, the experiment utilized G5(1C12)-hOKT3a Format 4 antibody having L1=L2=(GGGGS)₄ (SEQ ID NO: 3) with/or without the DSB44/100. It also utilized a G5(1C12)-hOKT3a Format 4 antibody with the shortened linker (10AAL=L1=L2=(GGGGS)₂) (SEQ ID NO: 4). The cell lines used to express the desired HLA-PEPTIDE targets were as follows: A375 cells (which express HLA subtype *01:01) engineered to express the G2 restricted peptide NTDNNLAVY (SEQ ID NO: 5), LN229 (which express HLA subtype B*35:01) engineered to express the G5 restricted peptide EVDPIGHVY (SEQ ID NO: 9). All cell lines were also engineered to express luciferase, using a lentivirus transduction of a cassette containing a 10× repeat of the peptide, Luciferase, and puromycin-resistance. Cassette-expressing cells were selected using 0.5 μg/mL of puromycin. For the assay, cells were pelleted, washed in PBS, and re-suspended at 2E6/mL in RPMI with 10% FBS. 25 μL of target cells were plated in opaque white 96-well plates. Serial dilutions of the bispecific molecules were added as described above. T cells were added to the plates to give a 10:1 T cell:target ratio as described above. Following 24-hour incubation, Bio-Glo luciferase substrate (Promega cat #G7941) was added and plate incubated and read according to manufacturer's instructions. To calculate % killing, RPMI background RLU was first subtracted from all values. % killing was determined as % cytotoxicity w/ Ab−% cytotoxicity w/o Ab, where % cytotoxicity was calculated as 100%−% viability. % viability was calculated as % of RLU in experimental wells normalized against target cells alone.

### Results

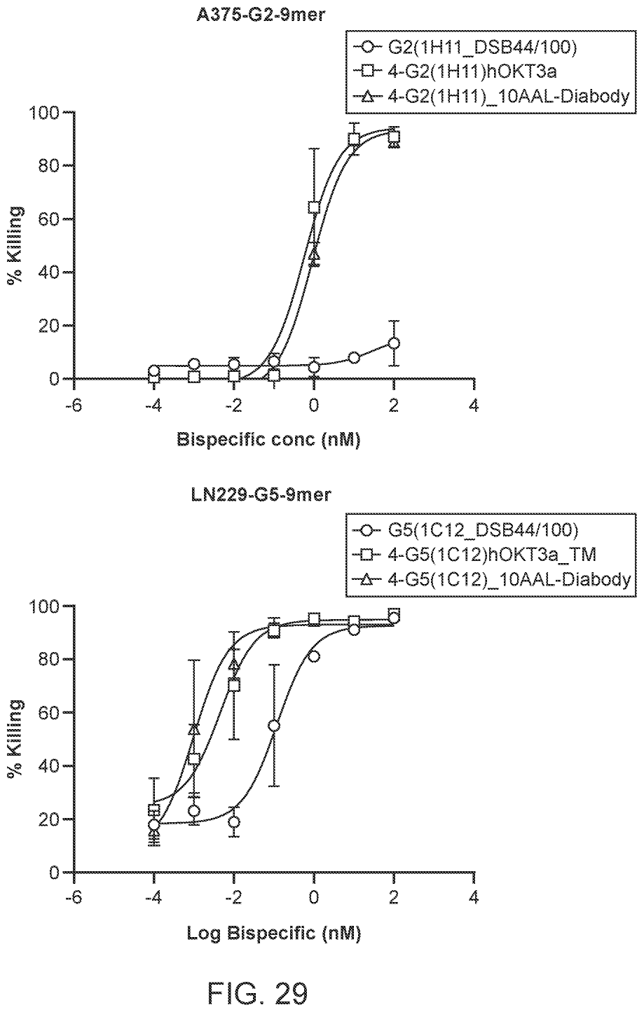

The results are shown in FIG. 29. All the G2 and G5 molecule exhibited varying degrees of cytotoxic potency. In the case of the G2 molecules, the G2(1H11)hOKT3a (non-shortened linkers; 2×scFv) and the G2(1H11)_10AAL (shortened linkers; diabody) groups showed significantly more cytotoxic potential than the DSB group. In the case of the G5 molecules, the cytotoxic potential of the G5(1C12)hOKT3a (non-shortened linkers; 2×scFv) and the G5(1C12)_10AAL (shortened linkers; diabody) groups showed significantly higher cytotoxic potential.

### Example 26: Digestion of Format 4 Antibodies with Engineered External DSBs

In order to stabilize molecules in the diabody format, without Fv modification, linkers were engineered that contain Cys residues to introduce disulfide bonds (DSBs) downstream of the diabody domains. First, the diabody conformation of the molecule was forced by shortening the VH-VL linker of the Fv on both chains to 10 amino acids (10AAL). It is known in the art that the C termini of the two chains that dimerize to form a diabody can exist in close or distal conformations relative to each other. See Olafsen, Tove, et al. Protein Engineering Design and Selection 17.1 (2004): 21-27, which is incorporated by reference in its entirety. By introducing a DSB immediately downstream of the second domain of each half-diabody construct, the proximal conformation is forced, and the overall assembly is stabilized. Constructs were generated that introduced Cys residues 3 amino acids (GGC) or 4 amino acids (GGGC (SEQ ID NO: 404)) downstream of the end of the diabody sequences. In the case of format 4-like molecules, after forming a DSB, the linkers both continue, either in the knob-Fc chain, or into hole-Fab-Fc chain as depicted in FIG. 30 (top right). Additionally, we designed constructs that disconnect one of the ABRs from the hole-Fab-Fc side and provide it as a separate chain in trans (FIG. 30; bottom). Similarly, we introduced Cys residues 3 amino acids (GGC) or 4 amino acids (GGGC (SEQ ID NO: 404)) downstream of the end of the diabody sequences, but in this case, the new 4th chain terminated after the Cys rather than continuing into the hole-Fab-Fc. Schematic diagrams of the assembled constructs are presented in FIG. 30, linkers are presented in Tables 39 and 40. In order to demonstrate that these sequences form diabody, they are subjected to FabALACTICA digestion and analyzed by SEC-HPLC and CE-SDS.

TABLE 39
Cysteine mutations in the linkers of ABP constructs that result in external DSBs.
Cysteines that form the DSBs are underlined.

| Construct | L1 & L2 | L3 | L4 |
|---|---|---|---|
| D- | GGGGSGGGG | GGGGSEPKSSDKTHT (SEQ | GGGGSGGGGS (SEQ |
| 1H11xhOKT3 | S (SEQ ID NO: | ID NO: 405) | ID NO: 107) |
| | 107) | | |
| D-1H11-LL- | GGGGSGGGG | GGCGGGGSGSEPKSSDKTHT | GGCGGGGSGSGGGGS |
| GGCxhOKT3 | S (SEQ ID NO: | (SEQ ID NO: 406) | (SEQ ID NO: 407) |
| | 107) | | |
| D-1H11-LS- | GGGGSGGGG | GGCGSEPKSSDKTHT (SEQ | GGCGGGGSGSGGGGS |
| GGCxhOKT3 | S (SEQ ID NO: | ID NO: 408) | (SEQ ID NO: 407) |
| | 107) | | |
| D-1H11-SL- | GGGGSGGGG | GGCGGGGSGSEPKSSDKTHT | GGCGS (SEQ ID NO: |
| GGCxhOKT3 | S (SEQ ID NO: | (SEQ ID NO: 406) | 409) |
| | 107) | | |
| D-1H11-LL- | GGGGSGGGG | GGGCGGGGSGSEPKSSDKTH | GGGCGGGGSGSGGGG |
| GGGCxhOKT3 | S (SEQ ID NO: | T (SEQ ID NO: 410) | S (SEQ ID NO: 411) |
| | 107) | | |
| D-1H11-LS- | GGGGSGGGG | GGGCGSEPKSSDKTHT (SEQ | GGGCGGGGSGSGGGG |
| GGGCxhOKT3 | S (SEQ ID NO: | ID NO: 412) | S (SEQ ID NO: 411) |
| | 107) | | |
| D-1H11-SL- | GGGGSGGGG | GGGCGGGGSGSEPKSSDKTH | GGGCGS (SEQ ID NO: |
| GGGCxhOKT3 | S (SEQ ID NO: | T (SEQ ID NO: 410) | 413) |
| | 107) | | |

TABLE 40
Cysteine mutations in the linkers of ABP constructs that result in external DSBs.
Cysteines that form the DSBs are underlined.

| Construct | L1 & L2 | L3 | L4 |
|---|---|---|---|
| D- | GGGGSGGGG | GGGGSEPKSSDKTHT (SEQ | GGGGSGGGGS (SEQ |
| 1H11xhOKT3 | S (SEQ ID NO: | ID NO: 405) | ID NO: 107) |
| | 107) | | |
| D-1H11-LL- | GGGGSGGGG | GGCGGGGSGSEPKSSDKTHT | GGCGGGGSGSGGGGS |
| GGCxhOKT3 | S (SEQ ID NO: | (SEQ ID NO: 406) | (SEQ ID NO: 407) |
| | 107) | | |
| D-1H11-LS- | GGGGSGGGG | GGCGSEPKSSDKTHT (SEQ | GGCGGGGSGSGGGGS |
| GGCxhOKT3 | S (SEQ ID NO: | ID NO: 408) | (SEQ ID NO: 407) |
| | 107) | | |
| D-1H11-SL- | GGGGSGGGG | GGCGGGGSGSEPKSSDKTHT | GGCGS (SEQ ID NO: |
| GGCxhOKT3 | S (SEQ ID NO: | (SEQ ID NO: 406) | 409) |
| | 107) | | |
| D-1H11-LL- | GGGGSGGGG | GGGCGGGGSGSEPKSSDKTH | GGGCGGGGSGSGGGG |
| GGGCxhOKT3 | S (SEQ ID NO: | T (SEQ ID NO: 410) | S (SEQ ID NO: 411) |
| | 107) | | |
| D-1H11-LS- | GGGGSGGGG | GGGCGSEPKSSDKTHT (SEQ | GGGCGGGGSGSGGGG |
| GGGCxhOKT3 | S (SEQ ID NO: | ID NO: 412) | S (SEQ ID NO: 411) |
| | 107) | | |
| D-1H11-SL- | GGGGSGGGG | GGGCGGGGSGSEPKSSDKTH | GGGCGS (SEQ ID NO: |
| GGGCxhOKT3 | S (SEQ ID NO: | T (SEQ ID NO: 410) | 413) |
| | 107) | | |

### Materials and Methods

### Proteolysis by FabALACTICA:

0.3-0.5 mg of G2(1H11) Format 4 with L1=L2=(G4S)x2 (SEQ ID NO: 4) and DSB introduced into linker regions as described above, is buffer exchanged from PBS pH 7.4 into 150 mM sodium phosphate buffer at pH 7.0. The samples are then concentrated to a volume of approximately 100 μL, with corresponding concentrations ranging from 35 mg/mL, loaded onto FabALACTICA microspin columns (Genovis), and incubated for 18 hr with end-over-end mixing. To collect the digested products, the columns are centrifuged at 1000×g for 1 min, followed by three additional rounds of elution using 100 μL PBS pH 7.4. The elution fractions are pooled (referred to as “digested pool”), and subsequently loaded onto a CaptureSelect ProteinA (Genovis) column and incubated for a minimum of 30 min with end-over-end mixing. The flowthrough is collected by centrifugation at 200×g for 1 min, followed by three wash steps with 100 μL PBS pH 7.4. The flowthrough and wash fractions are pooled, and are henceforth referred to as “‘Fab’ fraction”. The ProteinA bound fragments are eluted using 100 μL of 0.1M Glycine, pH 3 by centrifugation at 200×g for 1 min, and immediately neutralized with 10 μL 1M tris pH 8. Four additional elution steps are performed by centrifugation at 1000×g for 1 min, and neutralized immediately as described. The elution fractions are henceforth referred to as “‘FC’ Fraction”

### SEC-HPLC

Analytical SEC-HPLC are performed on an Agilent 1260 series HPLC system equipped with a degasser (G4225A), binary pump (G1312B), autosampler (G1329B), and diode array detector (DAD, G4212B). Approximately 60 to 100 μg of each untreated antibody, and 100 μL of the “Fab” fraction, and “Fc” fraction are loaded onto a TSKgel G3000 SWxl column (7.8 mm ID×30 cm) with the TSKgel G2000SWxl-G4000SWxl Guard Column in line from Tosoh Bioscience. The column is operated at 0.5 ml/min for 30 min in PBS, pH 7.4. The DAD is set to collect absorbance at 280 nm.

CE-SDS

Capillary gel electrophoresis is performed using the LabChip GXII Touch HT system (PerkinElmer), and samples are analyzed using the ProteinExpress 200 High Sensitivity assay (PerkinElmer, #CLS960008) under reducing and non-reducing conditions. 2 μg of each untreated antibody, and 5 μL of each of digested pool, digested “Fab” fraction, and digested “Fc” fraction are mixed with 7 μL of reducing or non-reducing denaturing solution, and incubated at 70° C. for 10-12 min. The reducing denaturing solution is prepared by adding 24.5 μL of 1M DTT to 700 μL of non-reducing denaturing solution provided in the kit. Denatured samples are diluted with 32 μL of MilliQ water, mixed well, and spun down prior to analysis. The Protein Express LabChip (PerkinElmer, #760499), and ladder were prepared according to manufacturer instructions.

### Results

SEC-HPLC analysis of undigested format 4 G2(1H11) shortened L1 and L2 linkers (L1=L2=(G4S)×2) (SEQ ID NO: 4), along with the incorporation of the DSB introduced into the linkers downstream of the diabody sequences are expected to show a single peak with retention time around 17.7-18 minutes. This retention time aligns with what is hypothesized to be the peak corresponding to the diabody conformation in the split-peak profile observed prior to shortening the linker. Due to stabilization, no protein will be recovered in the “Fab” fraction for the Format 4-like constructs, as shown in SEC-HPLC chromatograms, and reducing and non-reducing gels. By contrast, in the 4 chain constructs, the Fab will be liberated by digestion and the diabody will remain with the proteinA bindable fraction. Absence of bands corresponding to subassemblies or partially digested products following digestion under non-reducing conditions (other than free Fab in the case of the 4-chain constructs) will indicate that the disulfide bond formation was complete, and is effective at stabilizing the diabody conformation and preventing breathing. Additionally, the “Fc” fraction that results from digestion of this molecule will result in a single peak on the SEC-HPLC chromatogram which aligns with the retention time of the undigested molecule, corresponding to a clipped diabody for the format 4-like molecules and will be slightly right-shifted for the 4-chain constructs, due to removal of the Fab domain.

### Example 27: Effect of Engineered External DSBs on the Activity of Format 4 Antibodies

The ABPs described in Example 26 are further analyzed to determine functional activity.

First, their affinity to G2 pHLA is measured using a ForteBio Octet HTX in 96-channel mode with biolayer interferometry (BLI) detection. High Precision Streptavidin SAX biosensors (P/N 18-5117) are loaded into the instrument. Biotinylated G2-pHLA is captured on the SAX biosensor at 2 μg/mL and ran for 120 s in the assay buffer composed of 0.02% Tween-20 and 0.1% BSA. The biosensors are then dipped in assay buffer for a baseline. Subsequently, the biosensors are dipped into wells containing varying concentrations of the bispecific antibody samples (3.125, 6.25, 12.5, 25, 50, 100 and 200 nM) to measure the association rate for 50 seconds. The biosensors are finally dipped into wells containing assay buffer to measure the dissociation rate for another 50 seconds. Referencing is completed by having a biosensor with no immobilized ligand dipped into analyte. Kinetic data is processed with Octet™ software using a 1:1 kinetic model with errors within 10%, X2 below 3, and R2 above 0.9.

Second, their binding to cells expressing the G2 pHLA target or the CD3 target is measured by flow cytometry. A375 cells, which express HLA-A*01:01, are engineered to express the restricted peptide NTDNNLAVY (SEQ ID NO: 5) using a lentivirus transduction of a cassette containing a 10× repeat of the peptide, Luciferase, and puromycin-resistance. Cassette-expressing cells are selected using 0.5 μg/mL of puromycin. Jurkat E6-1 (ATCC TIB-152) and Jurkat T3.5 (ATCC TIB-153) cells are grown under standard tissue culture conditions. Cells are harvested, washed in PBS, and stained with eBioscience Fixable Viability Dye eFluor 450 for 15 minutes at room temperature. Following another wash in PBS+2% FBS, cells are resuspended with bispecifics at varying concentrations. Cells are incubated with bispecifics for 1 hour at 4° C. After another wash, PE-conjugated goat anti-human IgG secondary antibody (Jackson ImmunoResearch) is added at 1:100. After incubating at 4° C. for 45 minutes and washing in PBS+2% FBS, cells are resuspended in PBS+2% FBS and analyzed by flow cytometry. Flow cytometric analysis is performed on the Attune NxT Flow Cytometer (ThermoFisher) using the Attune NxT Software. Data is analyzed using FlowJo.

Finally, cytotoxicity is measured. The cell lines used to express the desired HLA-PEPTIDE targets are as follows: A375 cells (which express HLA subtype A*01:01) engineered to express the G2 restricted peptide NTDNNLAVY (SEQ ID NO: 5), LN229 (which express HLA subtype B*35:01) engineered to express the G5 restricted peptide EVDPIGHVY (SEQ ID NO: 9). All cell lines are also engineered to express luciferase, using a lentivirus transduction of a cassette containing a 10× repeat of the peptide, Luciferase, and puromycin-resistance. Cassette-expressing cells are selected using 0.5 μg/mL of puromycin. For the assay, cells are pelleted, washed in PBS, and re-suspended at 2e6/mL in RPMI with 10% FBS. 25 μL of target cells are plated in opaque white 96-well plates. Serial dilutions of the bispecific molecules are added as described above. T cells are added to the plates to give a 10:1 T cell:target ratio as described above. Following 24-hour incubation, Bio-Glo luciferase substrate (Promega cat #G7941) is added and plate incubated and read according to manufacturer's instructions. To calculate % killing, RPMI background RLU is first subtracted from all values. % killing is determined as % cytotoxicity w/ Ab−% cytotoxicity w/o Ab, where % cytotoxicity is calculated as 100%−% viability. % viability is calculated as % of RLU in experimental wells normalized against target cells alone.

### Example 28: Purification Based on Kappa Constant Domains and Avidity

The current experiments develops a method of selectively purifying heterodimer via Kappa-constant domain purification in a single step or reduced number of steps relative to standard purification methods. By engineering an asymmetric antibody such as format 4 or format 5, where one half of the molecule contains a light-chain which has Kappa-constant region (FIG. 31), resins such as KappaSelect and CaptureSelect KappaXP/CaptureSelect KappaXL which bind specifically to the Kappa-constant region can be used to selectively purify out the heterodimer by taking advantage of avidity to the resin. The method can use gradient elution and/or step elution where the elution buffer is selected to facilitate the gradient or step condition (e.g. salt concentration and/or pH level). These Kappa-constant binding resins have higher binding capacities than GMP-grade ProteinL-based resins, which is often used for Fab purifications (e.g. CaptoL).

Materials and Methods

Expression

G2 antibodies were expressed transiently using the Expi293 expression system (Life Technologies), and harvested on day 5. Harvested cell culture fluid was clarified by centrifugation (4000×g, 20 min) followed by 0.45 um and 0.2 um filtration. The resulting cell culture fluid is referred to as “non-spiked load”.

Spiked Load Preparation

Currently used expression conditions result in the knob-knob homodimer and light-chain dimer (FIG. 31) as the major product related impurities that need to be cleared via purification. Optimization of expression conditions (e.g. by codon optimization/changing to stable CHO cell line etc.) is expected to result in the commonly seen hole-hole homodimer impurity. To generate load material that contains this impurity, a purified hole-hole homodimer-like molecule (HH-homodimer mimic) was spiked into the format 4 cell culture fluid at a protein ratio of 1:3 (HH-homodimer mimic):Format 4. This generates a cell culture fluid that contains 25% HH-homodimer mimic, and 75% format 4 and associated product-related impurities (referred to as “spiked load”).

Purification

All purifications were performed on an AKTA Avant 25 system, using a 1 mL CaptureSelect KappaXP or CaptureSelect KappaXL column (0.8 cm ID×2 cm). The column was first equilibrated using 5 column volumes (CV) of PBS pH 7.4 at a 1 or 1.5 minute residence time (RT). Clarified cell culture fluid was then loaded at a 1.5 min RT upto a loading density of 10 mg/mL when using non-spiked load, and 5 mg/mL when using spiked load. For pH gradient runs, the column was washed with 5 CV 1×PBS pH 7.4 at a 1.5 min RT, followed by 5CV of 50 mM Sodium Acetate pH 6 buffer at a 3 min RT to bring the column into the buffer used to generate the top of the pH gradient. For salt gradient runs, the column was washed with 5CV 1×PBS pH 7.4 at a 1.5 min RT, followed by 5CV of 50 mM Sodium acetate, 200 mM NaCl pH 4.2 at a 3 minute residence time. The pH gradient was carried out at a 3 min RT using 50 mM Sodium acetate from pH 6 to pH 3 over 40 CV, and was followed by a 7 CV post-gradient elution hold step using the pH 3 gradient elution buffer. The salt gradients were carried out at a 3 min RT in 50 mM sodium acetate buffer at pH 4.2, 3.9, and 3.6, from 200 mM NaCl to 0 mM NaCl over 40 CV, and were followed by a 7 CV post-gradient elution hold step using the 0 mM NaCl gradient buffer. The column CIP was performed using 5CV 250 mM Sodium Acetate pH 2.5, 4CV 0.1M Sodium hydroxide, and re-equilibrated using 4CV 1×PBS before storage in 20% Ethanol.

CE-SDS

Capillary gel electrophoresis was performed using the LabChip GXII Touch HT system (PerkinElmer), and samples were analyzed using the ProteinExpress 200 High Sensitivity assay (PerkinElmer, #CLS960008) under non-reducing conditions. 5 μL of each of sample tested was mixed with 7 μL of reducing or non-reducing denaturing solution, and incubated at 70° C. for 10-12 min. Denatured samples were diluted with 32 μL of MilliQ water, mixed well, and spun down prior to analysis. The Protein Express LabChip (PerkinElmer, #760499), and ladder were prepared according to manufacturer instructions.

SEC-HPLC

Analytical SEC-HPLC was performed on an Agilent 1260 series HPLC system equipped with a degasser (G4225A), binary pump (G1312B), autosampler (G1329B), and diode array detector (DAD, G4212B). 100 μL of each sample tested was loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 30 min in PBS, pH 7.4. The DAD was set to collect absorbance at 280 nm. Eluate fractions obtained were pooled to create mock eluate pools, and 0.2 um filtered prior to analysis.

Results

When expressing each chain separately (i.e. knob, hole, and light-chain), the impurity profile expected consists of the knob-knob homodimer, hole-hole homodimer, and light-chain dimer. For the format 4 molecule the knob-knob homodimer has no Kappa-constant domain, and is thus lost to the flowthrough during purification. The heterodimer contains one Kappa-constant domain, and the hole-hole homodimer has two Kappa-constant domains (FIG. 31). The avidity effect of the hole-hole homodimer results in its detachment from the resin under stronger elution conditions than would be required for the heterodimer containing a single Kappa-constant domain. The light-chain dimer has two Kappa-constant domains as well, thus is expected to detach from the resin under stronger elution conditions than is required for the heterodimer as well.

Comparing CaptureSelect Kappa XP and CaptureSelect Kappa XL as Candidate Resins

CaptureSelect Kappa XL and CaptureSelect Kappa XP are commercially available GMP resins with VHH-based ligands that have been engineered to recognize the constant domain of Kappa light-chains, and enable elution at mildly low pH. (See, for example, International Application No. PCT/NL2005/000829 and U.S. application Ser. No. 11/792,145, each of which is incorporated by reference in its entirety). To determine the resolving power of the two resins, non-spiked G2 antibody format 4 load, containing the format 4 heterodimer, light-chain dimer and knob-knob homodimer, was applied to the column as described above and a pH gradient in sodium acetate buffer from pH 6 to pH 3 was performed during the elution step.

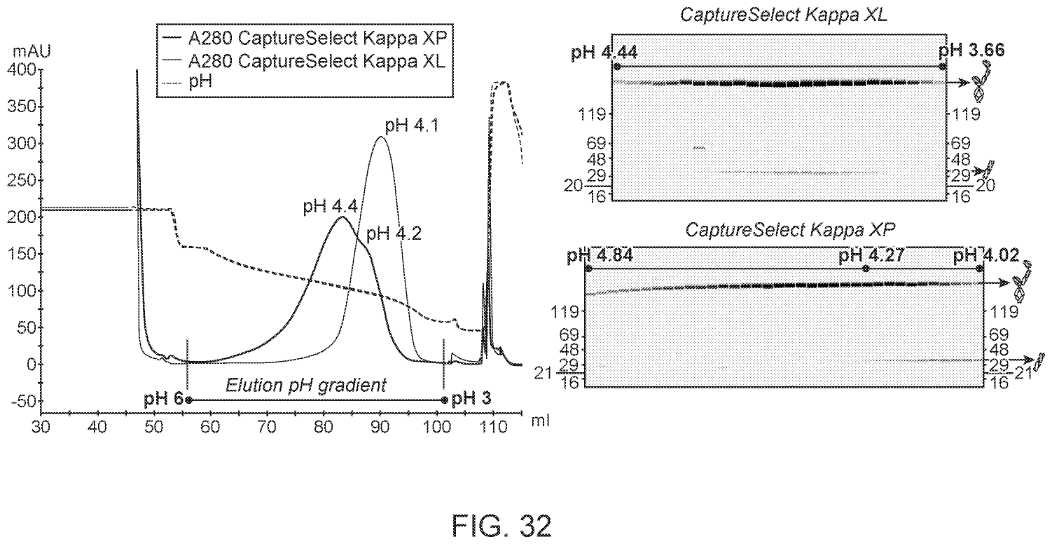

As shown in FIG. 32, a single peak, with UV max at pH 4.1 was observed for CaptureSelect KappaXL, while two peaks with UV max at pH 4.4 and pH 4.2 were observed for CaptureSelect KappaXP. Analysis of the fractions within these peaks by CE-SDS reveal that the light-chain dimer and desired format 4 heterodimer co-elute when using CaptureSelect KappaXL, while separation of these species was clearly achieved when using CaptureSelect KappaXP. For the latter, the format 4 molecule, which was monovalent for the Kappa constant domain, elutes first between pH 4.8 and pH 4.3, followed by the light-chain dimer. Both CaptureSelect Kappa XP and CaptureSelect Kappa XL eluates do not contain the knob-knob homodimer impurity, which was expected to be lost to the flowthrough. Additionally, separation of aggregates was also observed with the CaptureSelect KappaXP resin, where the first peak was found to contain 9% aggregate, while the shoulder/second peak contained 38% aggregate. Based on these observations, CaptureSelect KappaXP was found to have more resolving power to separate out the format 4 molecule from expected product related impurities, and was thus chosen as the candidate resin for further studies.

Performing pH Gradient Using Spiked Load

To determine whether separation between the commonly observed hole-hole homodimer impurity, light-chain dimer, and the desired format 4 molecule can be achieved using CaptureSelect Kappa XP, spiked load, described above, was applied to the column and eluted as a pH gradient from pH 6 to 3.

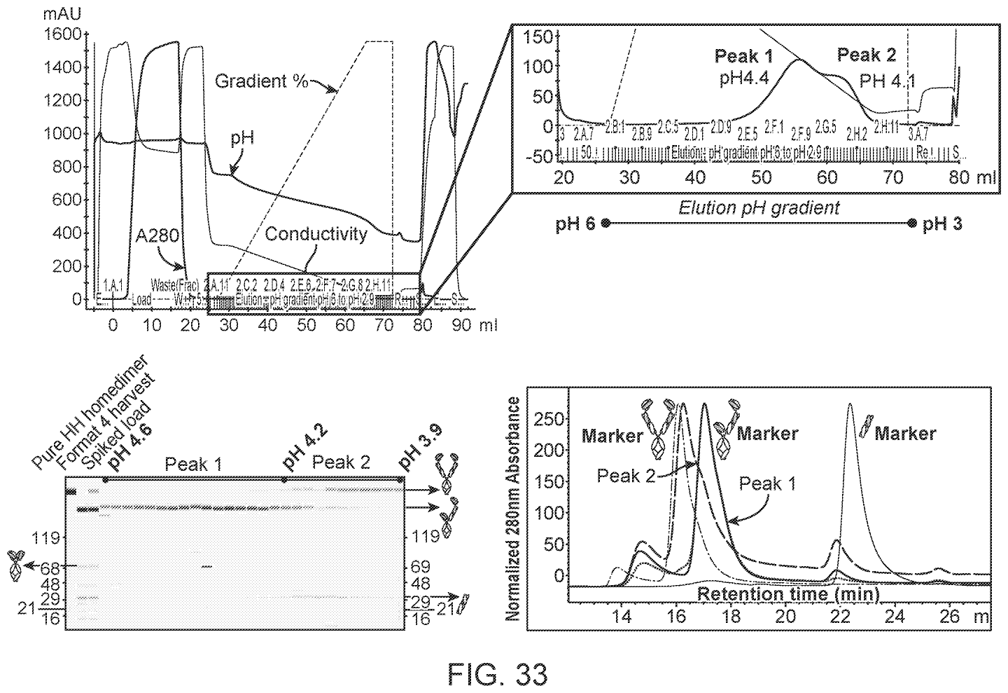

As shown in FIG. 33, the elution chromatogram reveals two peaks, with the UV max of the first peak (Peak 1) at pH 4.4, and the UV max of the second peak (Peak 2), at pH 4.1. CE-SDS analysis of the fractions collected over the gradient reveal that Peak 1 contains pure format 4, while Peak 2 contains the hole-hole homodimer and light-chain dimer impurities. No knob-knob homodimer was observed in the elution fractions. The knob-knob homodimer-like band that was present in one of the Peak 1 fractions appeared to be due to interference from a microbubble or dust particle present in the sample analyzed. SEC-HPLC analysis of pooled Peak 1 and Peak 2 mirror the CE-SDS analysis. Peak 1 SEC-HPLC trace was composed of a minor aggregate peak (12%) at a retention time around 14.5 minutes, a second minor peak that aligns with the hole-hole homodimer marker (4%), and a third major peak that aligned with the format 4 molecule marker. The SEC-HPLC trace for Peak 2 contains a minor aggregate peak, a minor peak that aligned with the light-chain dimer marker around 22.5 minutes, and a major peak that aligned with the hole-hole homodimer marker. These results further indicate that separation of product-related impurities from the desired format 4 molecule by exploiting valency of the Kappa-constant domain can be achieved.

Comparison of NaCl Gradients Using Spiked Load

Gradient elution-based chromatography processes work well in a laboratory setting. However, such processes tend not to be scalable, and are difficult to execute at commercial scale. It was thus important to determine optimal buffer conditions that selectively elute the heterodimeric format 4 molecule in a step elution. According to the pH gradient performed supra, candidate pH values were chosen, namely pH 4.2 (slightly more acidic than highest pH in range), 3.9, and 3.6 (slightly more acidic than lowest pH of range), at which NaCl gradients were performed in the elution step. Given that low pH was required for elution, and that resin data sheets mention that chaotropic agents may be used for elution at higher pH, affinity to the column was likely governed by electrostatic and hydrophobic interactions between the ligand and bound proteins. It was hypothesized that lowering pH results in sufficient charge repulsion to elute the protein, and that addition of NaCl would stabilize the charge repulsion, thus weakening the elution buffer at a given pH. The gradients were thus performed by lowering the concentration of NaCl from 200 mM to 0 mM, and the fractions were analyzed by CE-SDS and SEC HPLC. For all three runs performed, the protein sample applied to the column was spiked load.

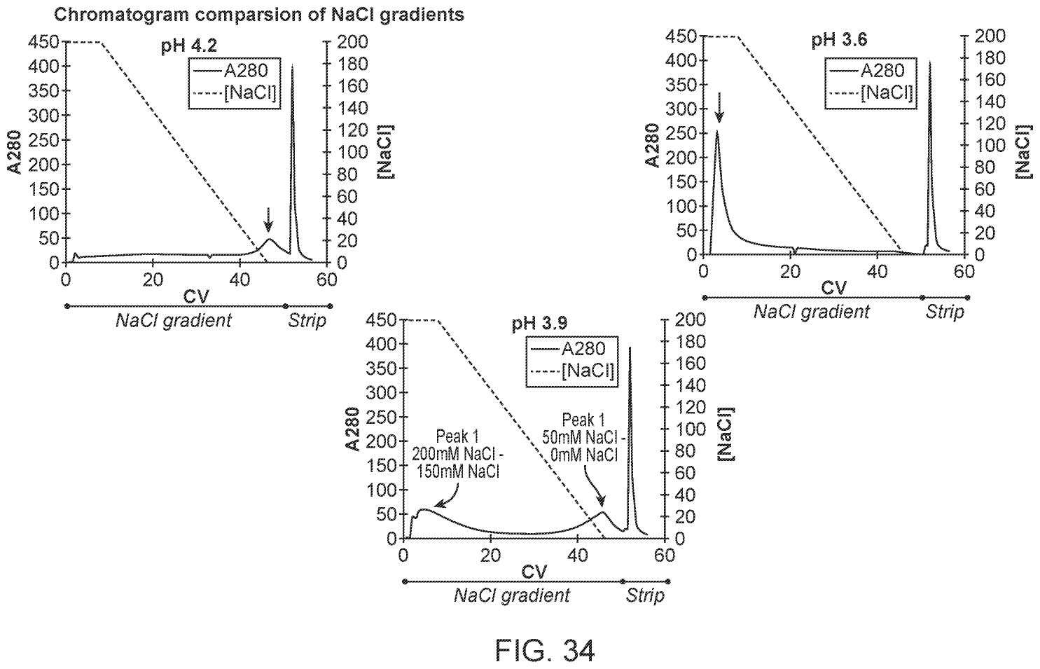

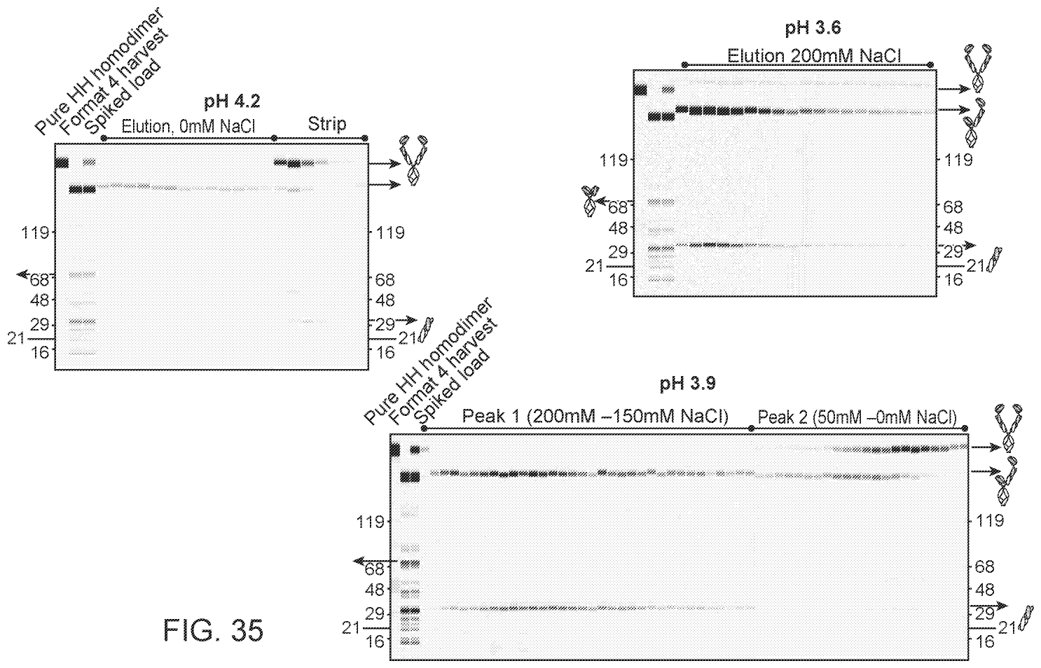

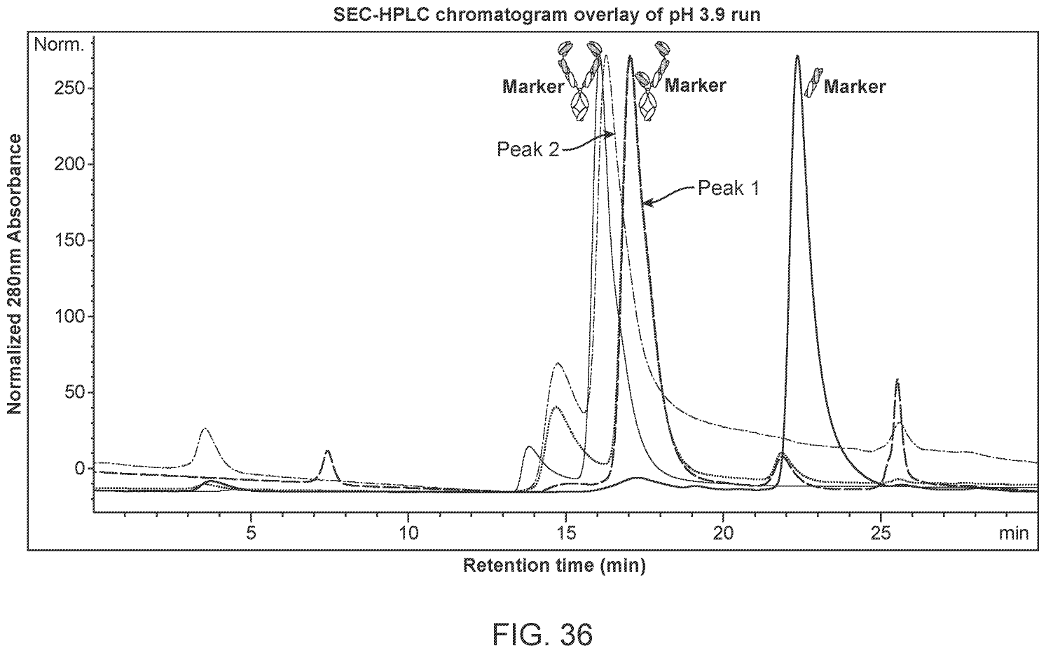

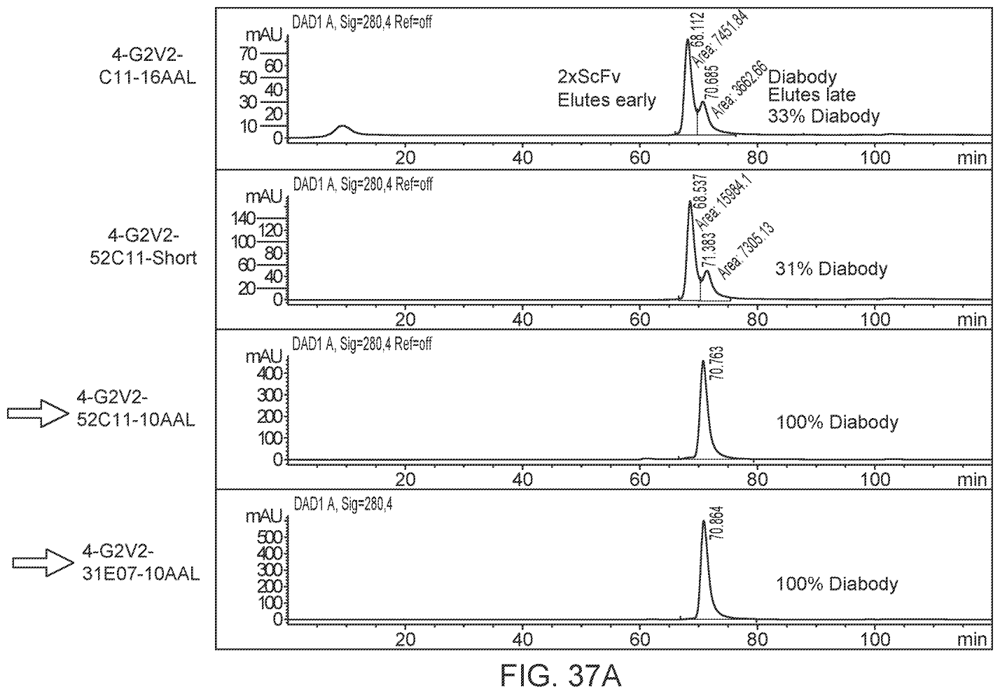

The results showed that the NaCl gradient at pH 3.9 was most effective at resolving between the hole-hole homodimer and format 4 molecules. The purification chromatograms show that elution only occurs in the absence of NaCl at pH 4.2 (FIG. 34, left), and that pH 3.6 was too strong to enable resolution between the homodimer impurities and heterodimeric format 4 molecule (FIG. 34, right). The gradient performed at pH 3.9 results in two peaks, with peak 1 ranging from 200 mM NaCl to 150 mM NaCl, and Peak 2 ranging from 50 mM NaCl to 0 mM NaCl (FIG. 34, middle). Analysis of the eluate fractions by CE-SDS revealed that the elution peak at pH 4.2 in the absence of salt contained pure product (FIG. 35), indicating that a step elution under these conditions would yield product with high purity, but recovery might be compromised. Peak 1 of the pH 3.9 NaCl gradient contained a mixture of the format 4 molecule as well as the light-chain dimer, while peak 2 was composed largely of the hole-hole homodimer (FIG. 35). SEC-HPLC analysis of pooled peak 1 further indicates that separation between the hole-hole homodimer and format 4 molecule was achieved as the chromatogram contained no peak with retention time that aligned with that of the hole-hole homodimer (FIG. 36). As suggested by the purification chromatogram, CE-SDS analysis of the eluate fractions at pH 3.6, 200 mM Sodium chloride peak showed that the product related impurities co-elute with the format 4 molecule, resulting in impure product under these conditions (FIG. 35).

These results collectively point towards the ability to achieve a step elution resulting in pure format 4 of reasonably high recovery while taking advantage of valency of the Kappa-constant domain on asymmetric molecules. The ideal buffer composition is expected to be a solution at pH 3.9 with NaCl concentration within the range of 150 mM and 50 mM, or alternatively a pH 4.2 solution with no NaCl. Residual light-chain dimer that is expected to co-elute with the format 4 molecules in a pH 3.9 solution is expected to be easily cleared by conventionally used ion-exchange or mixed-mode based polishing steps.

### Example 29: Identification of Predicted HLA-PEPTIDE Complexes (Table A)

We identified two classes of cancer specific HLA-peptide targets: The first class (cancer testis antigens, CTAs) are not expressed or are expressed at minimal levels in most normal tissues and expressed in tumor samples. The second class (tumor associated antigens, TAAs) are expressed highly in tumor samples and may have low expression in normal tissues.

We identified gene targets using three computational steps: First, we identified genes with low or no expression in most normal tissues using data available through the Genotype-Tissue Expression (GTEx) Project [1]. We obtained aggregated gene expression data from the Genotype-Tissue Expression (GTEx) Project (version V7p2). This dataset comprised 11,688 post-mortem samples from 714 individuals and fifty-three different tissue types. Expression was measured using RNA-Seq and computationally processed according to the GTEx standard pipeline (https://www.gtexportal.org/home/documentationPage). Gene expression was calculated using the sum of isoform expression that were calculated using RSEM v1.2.22 [2].

Next, we identified which of those genes are aberrantly expressed in cancer samples using data from The Cancer Genome Atlas (TCGA) Research Network: http://cancergenome.nih.gov/. We examined 11,093 samples available from TCGA (Data Release 6.0). Because GTEx and TCGA use different annotations of the human genome in their computational analyses, we only included genes for which there were available ENCODE mappings between the two datasets.

Finally, in these genes, we identified which peptides are likely to be presented as cell surface antigens by MHC Class I proteins using a deep learning model trained on HLA presented peptides sequenced by tandem mass spectrometry (MS/MS), as described in international patent application no. PCT/US2016/067159, herein incorporated by reference, in its entirety, for all purposes.

Specific criteria for the two classes of genes is given below.

CTA Inclusion Criteria

To identify the CTAs, we sought to define a criteria to exclude genes that were expressed in normal tissue that was strict enough to ensure tumor specificity, but would not exclude non-zero measurements arising from potential artifacts such as read misalignment. Genes were eligible for inclusion as CTAs if they met the following criteria: The median GTEx expression in each organ that was a part of the brain, heart, or lung was less than 0.1 transcripts per million (TPM) with no one sample exceeding 5 TPM. The median GTEx expression in other essential organs was less than 2 TPM with no one sample exceeding 10 TPM. Expression was ignored for organs classified as non-essential (testis, thyroid, and minor salivary gland). Genes were considered expressed in tumor samples if they had expression in TCGA of greater than 20 TPM in at least 30 samples.

We then examined the distribution of the expression of the remaining genes across the TCGA samples. When we examined the known CTAs, e.g. the MAGE family of genes, we observed that the expression these genes in log space was generally characterized by a bimodal distribution. This distribution consisted of a left mode around a lower expression value and a right mode (or thick tail) at a higher expression level. This expression pattern is consistent with a biological model in which some minimal expression is detected at baseline in all samples and higher expression of the gene is observed in a subset of tumors experiencing epigenetic dysregulation. We reviewed the distribution of expression of each gene across TCGA samples and discarded those where we observed only a unimodal distribution with no significant right-hand tail.

TAA Inclusion Criteria

The TAAs were identified by focusing on genes with much higher expression in tumor tissues than in normal tissue: We first identified genes with a median TPM of less than 10 in all GTEx essential, normal tissues and then selected the subset which had expression of greater than 100 TPM in at least one TCGA tumor tissues. Then, we examined the distribution of each of these genes and selected those with a bimodal distribution of expression, as well as additional evidence of significantly elevated expression in one or more tumor types.

Lists were further reviewed to eliminate genes which are known to have expression in tissues not adequately represented in GTEx or which could have originated from immune cell infiltrates within the tumor. These steps left of us with a final list of 56 CTA and 58 TAA genes.

We also added peptides from two additional proteins known to be present in cancer. We added the junction peptides from the EGFR-SEPT14 fusion protein [3] and we added peptides from KLK3 (PSA). We also added peptides from two genes from the same gene family as PSA: KLK2 and KLK4.

To identify the peptides that are likely to be presented as cell surface antigens by MHC Class I proteins, we used a sliding window to parse each of these proteins into its constituent 8-11 amino acid sequences. We processed these peptides and their flanking sequences with the HLA peptide presentation deep learning model to calculate the likelihood of presentation of each peptide at the max expression level observed for this gene in TCGA. We considered a peptide likely to be presented (i.e., a candidate target) if its quantile normalized probability of presentation calculated by our model was greater than 0.001.

The results are shown in Table A of International Application No. PCT/US2020/15736 or U.S. application Ser. No. 17/426,627, each of which is hereby incorporated by reference in its entirety. For clarity, each HLA-PEPTIDE was assigned a target number in Table A. For example, HLA-PEPTIDE target 1 is HLA-C*16:01_AAACSRMVI (SEQ ID NO: 414), HLA-PEPTIDE target 2 is HLA-C*16:02_AAACSRMVI (SEQ ID NO: 414), and so forth.

In summary, the example provides a large set of tumor-specific HLA-PEPTIDEs that can be pursued as candidate targets for ABP development.

### REFERENCES

1. Consortium, G. T., The Genotype-Tissue Expression (GTEx) project. Nat Genet, 2013. 45(6): p. 580-5.
2. Li B, Dewey C N., RSEM: accurate transcript quantificationfrom RNA-Seq data with or without a reference genome. BMC Bioinformatics. 2011 Aug. 4; 12:323.
3. Frattini V, Trifonov V, Chan J M, Castano A, Lia M, Abate F, Keir S T, Ji A X, Zoppoli P, Niola F, Danussi C, Dolgalev I, Porrati P, Pellegatta S, Heguy A, Gupta G, Pisapia D J, Canoll P, Bruce J N, McLendon R E, Yan H, Aldape K, Finocchiaro G, Mikkelsen T, Privé G G, Bigner D D, Lasorella A, Rabadan R, Iavarone A. The integrated landscape of driver genomic alterations in glioblastoma. Nat Genet. 2013 October; 45(10):1141-9.

### Example 30: Validation of Predicted HLA-PEPTIDE Complexes

The presence of peptides from the HLA-PEPTIDE complexes of Tables A, A1, and A2 was determined using mass spectrometry (MS) on tumor samples known to be positive for each given HLA allele from the respective HLA-PEPTIDE complex.

Isolation of HLA-peptide molecules was performed using classic immunoprecipitation (IP) methods after lysis and solubilization of the tissue sample (1-4). Fresh frozen tissue was first frozen in liquid nitrogen and pulverized (CryoPrep; Covaris, Woburn, MA). Lysis buffer (1% CHAPS, 20 mM Tris-HCl, 150 mM NaCl, protease and phosphatase inhibitors, pH=8) was added to solubilize the tissue and 1/10^{th} of the sample was aliquoted for proteomics and genomic sequencing efforts. The remainder of the sample was spun at 4° C. for 2 hrs to pellet debris. The clarified lysate was used for the HLA specific IP.

Immunoprecipitation was performed using antibodies coupled to beads where the antibody was specific for HLA molecules. For a pan-Class I HLA immunoprecipitation, the antibody W6/32 (5) was used, for Class II HLA-DR, antibody L243 (6) was used. Antibody was covalently attached to NHS-sepharose beads during overnight incubation. After covalent attachment, the beads were washed and aliquoted for IP. Additional methods for IP can be used including but not limited to Protein A/G capture of antibody, magnetic bead isolation, or other methods commonly used for immunoprecipitation.

The lysate was added to the antibody beads and rotated at 4° C. overnight for the immunoprecipitation. After immunoprecipitation, the beads were removed from the lysate and the lysate was stored for additional experiments, including additional IPs. The IP beads were washed to remove non-specific binding and the HLA/peptide complex was eluted from the beads with 2N acetic acid. The protein components were removed from the peptides using a molecular weight spin column. The resultant peptides were taken to dryness by SpeedVac evaporation and can be stored at −20° C. prior to MS analysis.

Dried peptides were reconstituted in HPLC buffer A and loaded onto a C-18 microcapillary HPLC column for gradient elution in to the mass spectrometer. A gradient of 0-40% B (solvent A—0.1% formic acid, solvent B—0.1% formic acid in 80% acetonitrile) in 180 minutes was used to elute the peptides into the Fusion Lumos mass spectrometer (Thermo). MS1 spectra of peptide mass/charge (m/z) were collected in the Orbitrap detector with 120,000 resolution followed by 20 MS2 scans. Selection of MS2 ions was performed using data dependent acquisition mode and dynamic exclusion of 30 sec after MS2 selection of an ion. Automatic gain control (AGC) for MS1 scans was set to 4×105 and for MS2 scans was set to 1×104. For sequencing HLA peptides, +1, +2 and +3 charge states can be selected for MS2 fragmentation. Alternatively, MS2 spectra can be acquired using mass targeting methods where only masses listed in the inclusion list were selected for isolation and fragmentation. This was commonly referred to as Targeted Mass Spectrometry and was performed in either a qualitative manner or can be quantitative. Quantitation methods require each peptide to be quantitated to be synthesized using heavy labeled amino acids. (Doerr 2013)

MS2 spectra from each analysis were searched against a protein database using Comet (7-8) and the peptide identification was scored using Percolator (9-11) or using the integrated de novo sequencing and database search algorithm of PEAKS. Peptides from targeted MS2 experiments were analyzed using Skyline (Lindsay K. Pino et al. 2017) or other method to analyze predicted fragment ions.

The presence of multiple peptides from the predicted HLA-PEPTIDE complexes was determined using mass spectrometry (MS) on various tumor samples known to be positive for each given HLA allele from the respective HLA-PEPTIDE complex.

Representative spectra data for selected HLA-restricted peptides is shown in PCT/US2020/1573, which is hereby incorporated by reference in its entirety. Each spectra contains the peptide fragmentation information as well as information related to the patient sample, including HLA types.

The spontaneous modification of amino acids can occur to many amino acids. Cysteine was especially susceptible to this modification and can be oxidized or modified with a free cysteine. Additionally N-terminal glutamine amino acids can be converted to pyro-glutamic acid. Since each of these modifications results in a change in mass, they can be definitively assigned in the MS2 spectra. To use these peptides in preparation of ABPs the peptide may need to contain the same modification as seen in the mass spectrometer. These modifications can be created using simple laboratory and peptide synthesis methods (Lee et al.; Ref 14).

### REFERENCES

(1) Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
(2) Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
(3) Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
(4) Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
(5) Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
(6) Goldman J M, Hibbin J, Kearney L, Orchard K, Th'ng KH. HLA-DR monoclonal antibodies inhibit the proliferation of normal and chronic granulocytic leukaemia myeloid progenitor cells. Br J Haematol. 1982 November; 52(3):411-20.
(7) Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4.
(8) Eng J K, Hoopmann M R, Jahan T A, Egertson J D, Noble W S, MacCoss MJ. A deeper look into Comet—implementation and features. J Am Soc Mass Spectrom. 2015 November; 26(11):1865-74. doi: 10.1007/s13361-015-1179-x. Epub 2015 Jun. 27.
(9) Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007.
(10) Lukas Käll, John D. Storey, Michael J. MacCoss and William Stafford Noble. Assigning confidence measures to peptides identified by tandem mass spectrometry. Journal of Proteome Research, 7(1):29-34, January 2008.
(11) Lukas Käll, John D. Storey and William Stafford Noble. Nonparametric estimation of posterior error probabilities associated with peptides identified by tandem mass spectrometry. Bioinformatics, 24(16):i42-i48, August 2008.
(12) Doerr, A. (2013) Mass Spectrometry-based targeted proteomics. Nature Methods, 10, 23.
(13) Lindsay K. Pino, Brian C. Searle, James G. Bollinger, Brook Nunn, Brendan MacLean & M. J. MacCoss (2017) The Skyline ecosystem: Informatics for quantitative mass spectrometry proteomics. Mass Spectrometry Reviews.
(14) Lee W Thompson; Kevin T Hogan; Jennifer A Caldwell; Richard A Pierce; Ronald C Hendrickson; Donna H Deacon; Robert E Settlage; Laurence H Brinckerhoff, Victor H Engelhard; Jeffrey Shabanowitz; Donald F Hunt; Craig L Slingluff. Preventing the spontaneous modification of an HLA-A2-restricted peptide at an N-terminal glutamine or an internal cysteine residue enhances peptide antigenicity. Journal of Immunotherapy (Hagerstown, Md.: 1997). 27(3):177-83, May 2004.

### Example 31: Identification of Antibodies and Antigen Binding Fragments Thereof that Bind HLA-PEPTIDE Targets

Overview

The following exemplification demonstrates that antibodies (Abs) can be identified that recognize tumor-specific HLA-restricted peptides. The overall epitope that is recognized by such Abs generally comprises a composite surface of both the peptide as well as the HLA protein presenting that particular peptide. Abs that recognize HLA complexes in a peptide-specific manner are often referred to as T cell receptor (TCR)-like Abs or TCR-mimetic Abs. Exemplary HLA-PEPTIDE targets included HLA-A*01:01_NTDNNLAVY (SEQ ID NO: 5) (HLA-PEPTIDE target “G2”), HLA-A*02:01_LLASSILCA (SEQ ID NO: 8) (HLA-PEPTIDE target “G7”), HLA-B*35:01_EVDPIGHVY (SEQ ID NO: 9) (HLA-PEPTIDE target “G5”), HLA-A*02:01_AIFPGAVPAA (SEQ ID NO: 6) (HLA-PEPTIDE target “G8”), and HLA-A*01:01_ASSLPTTMNY (SEQ ID NO: 7) (HLA-PEPTIDE target “G10”), respectively. Cell surface presentation of these HLA-PEPTIDE targets was confirmed by mass spectrometry analysis of HLA complexes obtained from tumor samples as described in Example 30. Representative plots are depicted in PCT/US2020/1573, which is hereby incorporated by reference in its entirety.

HLA-PEPTIDE Target Complexes and Counterscreen Peptide-HLA Complexes

The HLA-PEPTIDE targets G5, G8, G, as well as counterscreen negative control peptide-HLAs, were produced recombinantly using conditional ligands for HLA molecules using established methods. In all, 18 counterscreen HLA-peptides were generated for each of the HLA-PEPTIDE targets. The 18 counterscreen HLA-peptides were designed such that (A) the negative control peptide was known to be presented by the same LA subtype (i.e. the HLA-related controls) or (B) the negative control peptides were known to be presented by a different HLA subtype. The grouping of the target and the negative control peptide-HLA complexes for screen 1 have detailed sequence information provided in Table 1, and for screen 2 have detailed sequence information provided in Table 2. (See PCT/US2020/1573, which is hereby incorporated by reference in its entirety).

TABLE 1
HLA-PEPTIDE sequence design for Screen 1 negative control peptides
and ″G5″ target

| Group | HLA | Peptide | Gene | Target |
|---|---|---|---|---|
| G1 | HLA-A*02:01 | LLFGYPVYV (SEQ | | Neg Ctrl 1 |
| | | ID NO: 415) | | |
| | HLA-A*02:01 | GILGFVFTL (SEQ | | Neg Ctrl 2 |
| | | ID NO: 416) | | |
| | HLA-A*02:01 | FLLTRILTI (SEQ ID | | Neg Ctrl 3 |
| | | NO: 417) | | |
| G2 | HLA-A*01:01 | YSEHPTFTSQY | | Neg Ctrl 1 |
| | | (SEQ ID NO: 401) | | |
| | HLA-A*01:01 | VSDGGPNLY (SEQ | | Neg Ctrl 2 |
| | | ID NO: 419) | | |
| | HLA-A*01:01 | ATDALMTGY | | Neg Ctrl 3 |
| | | (SEQ ID NO: 420) | | |
| G3 | HLA-A*11:01 | IVTDFSVIK (SEQ | | Neg Ctrl 1 |
| | | ID NO: 421) | | |
| | HLA-A*11:01 | KSMREEYRK (SEQ | | Neg Ctrl 2 |
| | | ID NO: 422) | | |
| | HLA-A*11:01 | SSCSSCPLSK (SEQ | | Neg Ctrl 3 |
| | | ID NO: 423) | | |
| G4 | HLA-A*11:01 | ATIGTAMYK (SEQ | | Neg Ctrl 1 |
| | | ID NO: 424) | | |
| | HLA-A*11:01 | AVFDRKSDAK | | Neg Ctrl 2 |
| | | (SEQ ID NO: 425) | | |
| | HLA-A*11:01 | SIIPSGPLK (SEQ | | Neg Ctrl 3 |
| | | ID NO: 426) | | |
| G5 | HLA-B*35:01 | EVDPIGHVY (SEQ | MAGEA6 | Target |
| | | ID NO: 9) | | |
| | HLA-B*35:01 | IPSINVHHY (SEQ | | Neg Ctrl 1 |
| | | ID NO: 427) | | |
| | HLA-B*35:01 | EPLPQGOLTAY | | Neg Ctrl 2 |
| | | (SEQ ID NO: 428) | | |
| | HLA-B*35:01 | VPLDEDFRKY | | Neg Ctrl 3 |
| | | (SEQ ID NO: 429) | | |
| G6 | HLA-A*03:01 | RLRAEAQVK (SEQ | | Neg Ctrl 1 |
| | | ID NO: 430) | | |
| | HLA-A*03:01 | RLRPGGKKK (SEQ | | Neg Ctrl 2 |
| | | ID NO: 431) | | |
| | HLA-A*03:01 | QVPLRPMTYK | | Neg Ctrl 3 |
| | | (SEQ ID NO: 432) | | |

TABLE 2
HLA-PEPTIDE sequence design for Screen 2 negative control peptides, G8,
and G10 targets*

| Group | HLA | Peptide | Gene | Target |
|---|---|---|---|---|
| G7/G8^{‡} | A*02:01 | LLFGYPVYV (SEQ | | Neg Ctrl 1 |
| | | ID NO: 415) | | |
| | A*02:01 | GILGFVFTL (SEQ | | Neg Ctrl 2 |
| | | ID NO: 416) | | |
| | A*02:01 | FLLTRILTI (SEQ ID | | Neg Ctrl 3 |
| | | NO: 417) | | |
| G9 | A*24:02 | TYGPVFMCL (SEQ | | Neg Ctrl 1 |
| | | ID NO: 433) | | |
| | A*24:02 | RYLKDQQLL (SEQ | | Neg Ctrl 2 |
| | | ID NO: 434) | | |
| | A*24:02 | PYLFWLAAI (SEQ | | Neg Ctrl 3 |
| | | ID NO: 435) | | |
| G10 | A*01:01 | ASSLPTTMNY | MAGE3/6 | Target |
| | | (SEQ ID NO: 7) | | |
| | A*01:01 | YSEHPTFTSQY | | Neg Ctrl 1 |
| | | (SEQ ID NO: 401) | | |
| | A*01:01 | VSDGGPNLY (SEQ | | Neg Ctrl 2 |
| | | ID NO: 419) | | |
| | A*01:01 | ATDALMTGY | | Neg Ctrl 3 |
| | | (SEQ ID NO: 420) | | |
| G11 (=G3) | A*11:01 | IVTDFSVIK (SEQ | | Neg Ctrl 1 |
| | | ID NO: 421) | | |
| | A*11:01 | KSMREEYRK (SEQ | | Neg Ctrl 2 |
| | | ID NO: 422) | | |
| | A*11:01 | SSCSSCPLSK (SEQ | | Neg Ctrl 3 |
| | | ID NO: 423) | | |
| G12 (=G6) | A*03:01 | RLRAEAQVK (SEQ | | Neg Ctrl 1 |
| | | ID NO: 430) | | |
| | A*03:01 | RLRPGGKKK (SEQ | | Neg Ctrl 2 |
| | | ID NO: 431) | | |
| | A*03:01 | QVPLRPMTYK | | Neg Ctrl 3 |
| | | (SEQ ID NO: 432) | | |

### Generation and Stability Analysis of HLA-PEPTIDE Target Complexes and Counterscreen Peptide-HLA Complexes

Results for the G5 counterscreen “minipool” and G2 target are shown in PCT/US2020/1573, which is hereby incorporated by reference in its entirety. All three counterscreen peptides and the G5 peptide rescued the HLA complex from dissociation.

Results for the additional G5 “complete” pool counterscreen peptides are shown in PCT/US2020/1573, which is hereby incorporated by reference in its entirety, demonstrating that they also form stable HLA-peptide complexes.

Results for counterscreen peptides and G8 target are shown in International Application No. PCT/US2020/1573, which is hereby incorporated by reference in its entirety. All three counterscreen peptides and the G8 peptide rescued the HLA complex from dissociation.

Results for the G10 counterscreen “minipool” and G10 target are shown in International Application No. PCT/US2020/1573, which is hereby incorporated by reference in its entirety. All three counterscreen peptides and the G10 peptide rescued the HLA complex from dissociation.

Results for the additional G8 and G10 “complete” pool counterscreen peptides are shown in International Application No. PCT/US2020/1573, which is hereby incorporated by reference in its entirety, demonstrating that they also form stable HLA-peptide complexes.

Phage Library Screening

The highly diverse SuperHuman 2.0 synthetic naïve scFv library from Distributed Bio Inc was used as input material for phage display, which has a 7.6×10¹⁰ total diversity on ultra-stable and diverse VH/VL scaffolds. For both screen 1 and screen 2 three to four rounds of bead-based phage panning with the target pHLA complex (as shown in Table 3) were conducted using established protocols to identify scFv binders to pHLAs G5, G8 and G10, respectively. (See International Application No. PCT/US2020/1573, which is hereby incorporated by reference in its entirety). For each round of panning, the phage library was initially depleted with 18 pooled negative pHLA complexes prior to the binding step with the target pHLAs. The phage titer was determined at every round of panning to establish removal of non-binding phage. The output phage supernatant was also tested for target binding by ELISA and suggested progressive enrichment of G5-, G8 and G10 binding phage (see International Application No. PCT/US2020/1573, which is hereby incorporated by reference in its entirety).

TABLE 3
Phage library screening strategy

| Round | Antigen concentration | Washes |
|---|---|---|
| R1 | 100 pmol | 3× PBST + 3× PBS (5 min washes) |
| R2 | 25 pmol | 5 PBST (2× 30 sec, 3× 5 min) + |
| | | 5 PBS (2× 30 sec, 3× 5 min) |
| R3 | 10 pmol | 8 PBST (4× 30 sec, 4× 5 min) + |
| | | 8 PBS (4× 30 sec, 4× 5 min) |
| R4 | 5 pmol, 10 pmol | 30 min PBST + 30 min PBS |

Bacterial periplasmic extracts (PPEs) of individual output clones were subsequently generated in 96-well plates using well-established protocols. The PPEs were used to test for binding to the target pHLA antigen by high throughput PPE ELISA. Positive clones were sequenced and re-arrayed to select sequence-unique clones. Sequence unique clones were then tested in a secondary ELISA for binding to target pHLA versus the panel of HLA-matched negative control pHLA complexes, thus establishing target specificity. The G8 negative control HLA complexes (i.e. A*24:02) did not HLA-match with the G8 target HLA complex (i.e. A*02:01). Therefore, HLA-A*02:01 complexes presenting the peptides LLFGYPVYV (SEQ ID NO: 415), GILGFVFTL (SEQ ID NO: 416) or FLLTRILTI (SEQ ID NO: 417) from G7 were used as HLA-matched minipool of negative controls for G8 in further biochemical and functional characterization assays for the TCR-mimetic Abs retrieved from the scFv library.

Isolation of scFv Hits

Individual, soluble scFv protein fragments were produced and purified for the scFv clones that were found to be selective when expressed in PPEs. As shown by scFv PPE ELISA, these clones exhibited at least three-fold selective binding to the target pHLA as compared to binding to the minipool of negative control pHLAs. Soluble scFv production allowed for further biochemical and functional characterization.

The resulting VH and VL sequences for the scFvs that bind target G5 are shown in Table 4. To clarify the organization of Table 4, and other Tables of scFv sequences, each scFv was assigned a clone name. For all clone names, clone names recite the target (e.g., G5), the plate number (e.g., plate 7), and well number (e.g., well E7) of the 96-well plate from which the clone was originally picked. For example, clone names, G5-P7E07, G5-7E7, G5(7E7), G5(7E07), all refer to the same scFv clone. For example, in Table 4, the scFv from clone G5(7E07) has the VH sequence

(SEQ ID NO: 141)
QVQLVQSGAEVKKPGASVKVSCKASGYTFTSYDINWVRQAPGQGLEWMG
IINPRSGSTKYAQKFQGRVTMTRDTSTSTVYMELSSLRSEDTAVYYCAR
DGVRYYGMDVWGQGTTVTVSS
and
the VL sequence
(SEQ ID NO: 159)
DIVMTQSPLSLPVTPGEPASISCRSSQSLLHSNGYNYLDWYLQKPGQSP
QLLIYLGSYRASGVPDRFSGSGSGTDFTLKISRVEAEDVGVYYCMQGLQ
TPITFGQGTRLEIK.

The resulting CDR sequences for the scFvs that bind target G5 are shown in Table 5. To clarify the organization of Table 5, each scFv was assigned a clone name in Table 5. For example, the scFv from clone G5(7E07) has an HCDR1 sequence that is YTFTSYDIN (SEQ ID NO: 436), an HCDR2 sequence that is GIINPRSGSTKYA (SEQ ID NO: 437), an HCDR3 sequence that is CARDGVRYYGMDVW (SEQ ID NO: 67), an LCDR1 sequence that is RSSQSLLHSNGYNYLD (SEQ ID NO: 438), an LCDR2 sequence that is LGSYRAS (SEQ ID NO: 439), and an LCDR3 sequence that is CMQGLQTPITF (SEQ ID NO: 85), according to the Kabat numbering system.

The resulting VH and VL sequences for the scFvs that bind target G8 are shown in Table 6. Table 6 is organized similarly to Table 4.

The resulting CDR sequences for the scFvs that bind target G8 are shown in Table 7. Table 7 is organized similarly to Table 5.

The resulting VH and VL sequences for the scFvs that bind target G10 are shown in Table 8. Table 8 is organized similarly to Table 4.

The resulting CDR sequences for the scFvs that bind target G10 are shown in Table 9. Table 9 is organized similarly to Table 5.

Resulting VH and VL sequences for scFvs that bind target G2 are shown in Table 27. Table 27 is organized similarly to Table 4.

Resulting CDR sequences for scFvs that bind target G2 are shown in Table 28. Table 28 is organized similarly to Table 5.

Resulting VH and VL sequences for scFvs that bind target G7 are shown in Table 29. Table 29 is organized similarly to Table 4.

Resulting CDR sequences for scFvs that bind target G7 are shown in Table 30. Table 30 is organized similarly to Table 5.

A number of clones were formatted into scFv, Fab, and IgG to facilitate biochemical, structural, and functional characterization (see Table 10).

TABLE 10
Hit rate of the screening campaigns. Clones were reformatted into (a)
IgG for biochemical and functional characterization, (b) Fab constructs
for protein crystallography and HDX mass spectrometry, and (c) scFv
constructs for HDX mass spectrometry.

| Group | G5 | G8 | G10 |
|---|---|---|---|
| HLA | B*35:01 | A*02:01 | A*01:01 |
| Peptide | MAGEA6 | FOXE1 | MAGE3/6 |
| Sequence Unique | 81 | 17 | 23 |
| Binders | | | |
| Selective Binders | 18 | 17 | 18 |
| IgG | 18 | 17 | 18 |
| Fab | 4 | 3 | 2 |
| scFv | 8 | 7 | 6 |

The results depict a flow chart describing the antibody selection process, including criteria and intended application for the scFv, Fab, and IgG formats (International Application No. PCT/US2020/1573, which is hereby incorporated by reference in its entirety). Briefly, clones were selected for further characterization based on sequence diversity, binding affinity, selectivity, and CDR3 diversity.

To assess sequence diversity, dendrograms were produced using clustal software. The predicted 3D structures of the scFv sequences, based on the VH type, were also taken into consideration. Binding affinity as determined by the equilibrium dissociation constant (K_{D}) was measured using an Octet HTX (ForteBio). Selectivity for the specific peptide-HLA complexes was determined with an ELISA titration of the purified scFvs as compared to the minipool of negative control pHLA complexes or streptavidin alone. Cutoff values for the K_{D} and selectivity were determined for each target set based on the range of values obtained for the Fabs within each set. Final clones were selected based on diversity in sequence families and CDR3 sequences.

The overall number of hits following phage library screening and scFv isolation are listed in Table 10, above.

Materials and Methods

HLA Expression and Purification:

Recombinant proteins were obtained through bacterial expression using established procedures (Garboczi, Hung, & Wiley, 1992). Briefly, the α chain and β2 microglobulin chain of various human leukocyte antigens (HLA) were expressed separately in BL21 competent E. coli cells (New England Biolabs). Following auto-induction, cells were lysed via sonication in Bugbuster® plus benzonase protein extraction reagent (Novagen). The resulting inclusion bodies were washed and sonicated in wash buffer with and without 0.5% Triton X-100 (50 mM Tris, 100 mM NaCl, 1 mM EDTA). After the final centrifugation, inclusion pellets were dissolved in urea solution (8 M urea, 25 mM MES, 10 mM EDTA, 0.1 mM DTT, pH 6.0). Bradford assay (Biorad) was used to quantify the concentration and the inclusion bodies were stored at −80° C.

Refold of pHLA and Purification:

HLA complexes were obtained by refolding of recombinantly produced subunits and a synthetically obtained peptide using established procedures (Garboczi et al., 1992). Briefly, the purified α and β2 microglobulin chains were refolded in refold buffer (100 mM Tris pH 8.0, 400 mM L-Arginine HCl, 2 mM EDTA, 50 mM oxidized glutathione, 5 mM reduced glutathione, protease inhibitor tablet) with either the target peptide or a cleavable ligand. The refold solution was concentrated with a Vivaflow 50 or 50R crossflow cassette (Sartorius Stedim). Three rounds of dialyses in 20 mM Tris pH 8.0 were performed for at least 8 hours each. For the antibody screening and functional assays, the refolded HLA was enzymatically biotinylated using BirA biotin ligase (Avidity). Refolded protein complexes were purified using a HiPrep (16/60 Sephacryl 5200) size exclusion column attached to an AKTA FPLC system. Biotinylation was confirmed in a streptavidin gel-shift assay under non-reducing conditions by incubating the refolded protein with an excess of streptavidin at room temperature for 15 minutes prior to SDS-PAGE. The peptide-HLA complexes were aliquoted and stored at −80° C.

Peptide Exchange:

HLA-peptide stability was assessed by conditional ligand peptide exchange and stability ELISA assay. Briefly, conditional ligand-HLA complexes were subjected to conditional stimulus in the presence or absence of the counterscreen or test peptides. Exposure to the conditional stimulus cleaves the conditional ligand from the HLA complex, resulting in dissociation of the HLA complex. If the counterscreen or test peptide stably binds the α1/α2 groove of the HLA complex, it “rescues” the HLA complex from disassociation. In short, a mixture of 100 μL of 50 μM of the novel peptide (Genscript) and 0.5 μM recombinantly produced cleavable ligand-loaded HLA in 20 mM Tris HCl and 50 mM NaCl at pH 8 was placed on ice. The mixture was irradiated for 15 min in a UV cross-linker (CL-1000, UVP) equipped with 365-nm UV lamps at ˜10 cm distance.

MHC Stability Assay:

The MHC stability ELISA was performed using established procedures. (Chew et al., 2011; Rodenko et al., 2006) A 384-well clear flat bottom polystyrene microplate (Corning) was precoated with 50 μl of streptavidin (Invitrogen) at 2 μg/mL in PBS. Following 2 h of incubation at 37° C., the wells were washed with 0.05% Tween 20 in PBS (four times, 50 μL) wash buffer, treated with 50 μl of blocking buffer (2% BSA in PBS), and incubated for 30 min at room temperature. Subsequently, 25 μl of peptide-exchanged samples that were 300× diluted with 20 mM Tris HCl/50 mM NaCl were added in quadruplicate. The samples were incubated for 15 min at RT, washed with 0.05% Tween wash buffer (4×50 L), treated for 15 min with 25 μL of HRP-conjugated anti-β2m (1 μg/mL in PBS) at RT, washed with 0.05% Tween wash buffer (4×50 μL), and developed for 10-15 min with 25 L of ABTS-solution (Invitrogen). The reactions were stopped by the addition of 12.5 μL of stop buffer (0.01% sodium azide in 0.1 M citric acid). Absorbance was subsequently measured at 415 nm using a spectrophotometer (SpectraMax i3x; Molecular Devices).

Phage Panning:

For each round of panning, an aliquot of starting phage was set aside for input titering and the remaining phage was depleted three times against Dynabead M-280 streptavidin beads (Life Technologies) followed by a depletion against Streptavidin beads pre-bound with 100 pmoles of pooled negative peptide-HLA complexes. For the first round of panning, 100 pmoles of peptide-HLA complex bound to streptavidin beads was incubated with depleted phage for 2 hours at room temperature with rotation. Three five-minute washes with 0.5% BSA in 1×PBST (PBS+0.05% Tween-20) followed by three five-minute washes with 0.5% BSA in 1×PBS were utilized to remove any unbound phage to the peptide-HLA complex bound beads. To elute the bound phage from the washed beads, 1 mL 0.1M TEA was added and incubated for 10 minutes at room temperature with rotation. The eluted phage was collected from the beads and neutralized with 0.5 mL 1M Tris-HCl pH 7.5. The neutralized phage was then used to infect log growth TG-1 cells (OD₆₀₀=0.5) and after an hour of infection at 37° C., cells were plated onto 2YT media with 100 μg/mL carbenicillin and 2% glucose (2YTCG) agar plates for output titer and bacterial growth for subsequent panning rounds. For subsequent rounds of panning, selection antigen concentrations were lowered while washes increased by amount and length of wash times at show in Table 3.

Input/Output Phage Titer:

Each round of input titer was serially diluted in 2YT media to 10¹⁰. Log phase TG-1 cells are infected with diluted phage titers (10⁷-10¹⁰) and incubated at 37° C. for 30 minutes without shaking followed by another 30 minutes with gentle shaking. Infected cells are plated onto 2YTCG plates and incubated overnight at 30° C. Individual colonies were counted to determine input titer. Output titers were performed following 1 h infection of eluted phage into TG-1 cells. 1, 0.1, 0.01, and 0.001 μL of infected cells were plated onto 2YTCG platers and incubated overnight at 30° C. Individual colonies were counted to determine output titer.

Selective Target Binding of Bacterial Periplasmic Extracts:

For scFv PPE ELISAs, 96-well and/or 384-well streptavidin coated plates (Pierce) were coated with 2 μg/mL peptide-HLA complex in HLA buffer and incubated overnight at 4° C. Plates were washed three times between each step with PBST (PBS+0.05% Tween-20). The antigen coated plates were blocked with 3% BSA in PBS (blocking buffer) for 1 hour at room temperature. After washing, scFv PPEs were added to the plates and incubated at room temperature for 1 hour. Following washing, mouse anti-v5 antibody (Invitrogen) in blocking buffer was added to detect scFv and incubated at room temperature for 1 hour. After washing, HRP-goat anti-mouse antibody (Jackson ImmunoResearch) was added and incubated at room temperature for 1 hour. The plates were then washed three times with PBST and 3 times with PBS before HRP activity was detected with TMB 1-component Microwell Peroxidase Substrate (Seracare) and neutralized with 2N sulfuric acid.

For negative peptide-HLA complex counterscreening, the scFv PPE ELISAs were performed as described above, except for the coating antigen. Namely, the HLA mini-pools (see Tables 1 and 2) were used that consisted of 2 μg/mL of each of the three negative peptide-HLA complexes pooled and coated onto streptavidin plates for comparison binding to their particular pHLA complex. Alternatively, HLA complete pools consisted of 2 μg/mL of each of all 18 negative peptide-HLA complexes pooled together and coated onto streptavidin plates for comparison binding to their particular pHLA complex.

Construction and Production of scFv Protein Fragments:

The expression plasmid was transformed into BL21(DE3) strain and co-expressed with a periplasmid chaperone in a 400 mL E. coli culture. The cell pellet was reconstituted as follows: 10 mL/1 g biomass with (25 mM HEPES, pH7.4, 0.3M NaCl, 10 mM MgCl2, 10% glycerol, 0.75% CHAPS, 1 mM DTT) plus lysozyme, and benzonase and Lake Pharma protease inhibitor cocktail. The cell suspension was incubated on a shaking platform at RT for 30 minutes. Lysates were clarified by centrifugation at 4° C., 13,000× rpm for 15 min. The clarified lysate was loaded onto 5 mL of Ni NTA resin pre-equilibrated in IMAC Buffer A (20 mM Tris-HCl, Ph7.5; 300 mM NaCl/10% Glycerol/1 mM DTT). The resin was washed with 10 column volumes (CVs) of Buffer A (or until a stable baseline was reached), followed by 10 CVs of 8% IMAC Buffer B (20 mM Tris-HCl, Ph7.5; 300 mM NaCl/10% Glycerol/1 mM DTT/250 mM Imidazole). The target protein was eluted in a 20CV gradient to 100% IMAC Buffer B. The column was washed with 5CVs of 100% IMAC B to ensure complete protein removal. Elution fractions were analyzed by SDS-PAGE and Western blot (anti-His) and pooled accordingly. The pool was dialyzed with the final formulation buffer (20 mM Tris-HCl, Ph7.5; 300 mM NaCl/10% glycerol/1 mM DTT), concentrated to a final protein concentration >0.3 mg/mL, aliquoted into 1 mL vials, and flash frozen in liquid nitrogen. Final QC steps included SDS-PAGE and A280 absorbance measurements.

Construction and Production of Fab Protein Fragments:

The constructs of selected G5, G8 and G10 Fabs were cloned into a vector optimized for mammalian expression. Each DNA construct was scaled up for transfection and sequences were confirmed. A 100 mL transient production was completed in HEK293 cells (Tuna293™ Process) for each. The proteins were purified by anti-CH1 purification subsequently purified by size exclusion chromatography (SEC) via HiLoad 16/600 Superdex 200. The mobile phase used for SEC-polishing was 20 mM Tris, 50 mM NaCl, pH 7. Final confirmatory CE-SDS analysis was performed.

Construction and Production of IgG Proteins:

The expression constructs of the G series antibodies were cloned into a vector optimized for mammalian expression. Each DNA construct was scaled up for transfection and sequences were confirmed. A 10 mL transient production was completed in HEK293 cells (Tuna293™ Process) for each. The proteins were purified by Protein A purification and final CE-SDS analysis was performed.

### Example 32: Affinity of Fab Clones for their Respective HLA-PEPTIDE Targets

Fab-formatted antibodies allow for accurate assessment of monomeric binding to their respective HLA-PEPTIDE targets, while avoiding confounding effects of bivalent interactions with the IgG antibody format. Binding affinity was assessed by bio-layer interferometry (BLI) using an Octet Qke (ForteBio). Briefly, biotinylated pHLA complexes in kinetics buffer were loaded onto streptavidin sensors for 300 seconds, at concentrations which gave the optimal nm shift response (approximately 0.6 nm) for each Fab at the highest concentration used. The ligand-loaded tips were subsequently equilibrated in the kinetics buffer for 120 seconds. The ligand-loaded biosensors were then dipped for 200 seconds in the Fab solution titrated into 2-fold dilutions. Starting Fab concentrations ranged from 100 nM to 2 μM, iteratively optimized based on the K_{D} values of the Fab. The dissociation step in the kinetics buffer was measured for 200 seconds. Data were analyzed using the ForteBio data analysis software using a 1:1 binding model.

Results for HLA-PEPTIDE targets HLA-1B*35:01_EVDPIGHVY (SEQ ID NO: 9), HLA-A*02:01_AIFPGAVPAA (SEQ ID NO: 6), and HLA-A*01:01_ASSLPTTMNY (SEQ ID NO: 7) are shown in Table 11, below. The Fab-formatted antibodies bind to their respective HLA-PEPTIDE targets with high affinity.

TABLE 11
Optimized Octet BLI affinity measurements of Fabs binding to
their target complex

| Target | Fab clone | KD (M) | Kon (1/Ms) | Kdis (1/s) | Full R\hat{ }2 |
|---|---|---|---|---|---|
| G5 | G5(7A05) | 1.19E−07 | 4.10E+05 | 4.87E−02 | 0.997 |
| G5 | G5(7B03) | 2.54E−07 | 4.42E+05 | 9.09E−02 | 0.993 |
| G5 | G5(7E07) | 2.82E−08 | 9.02E+05 | 2.48E−02 | 0.991 |
| G5 | G5(7F06) | 3.37E−08 | 9.15E+05 | 3.06E−02 | 0.995 |
| G8 | G8(2C10) | 1.77E−08 | 7.50E+04 | 1.30E−03 | 0.997 |
| G8 | G8(1C11) | 1.78E−07 | 1.90E+05 | 3.38E−02 | 0.997 |
| G8 | G8(2E04) | 2.86E−07 | 5.45E+05 | 7.89E−02 | 0.842 |
| G10 | G10(1B07) | 3.75E−08 | 1.65E+05 | 6.15E−03 | 0.997 |
| G10 | G10(4E07) | 4.28E−07 | 4.77E+05 | 1.11E−01 | 0.990 |

The results depict BLI results for Fab clone G5(7A05) to HLA-PEPTIDE target B*35:01-EVDPIGHVY (SEQ ID NO: 9) (11A), Fab clones G8(2C10) and G8(1C11) to HLA-PEPTTDE target A*02:01AFPGAVPAA (SEQ ID NO: 6) (1/s, 2C10 on left and C1 on right), and Fab clone G10(1407) to 8LA-PEPTIDE target A*01:01-ASSLPTTMNY (SEQ ID NO: 7) (11C), respectively. (See International Application No. PCT/US2020/1573, which is hereby incorporated by reference in its entirety).

The results show BLI results for G2 target Fab clone G2(1H11) and for G7 target Fab clone G7(2E09), respectively. (See International Application No. PCT/US2020/1573, which is hereby incorporated by reference in its entirety).

Results are shown in the Table below.

TABLE 31
Optimized Octet BLI affinity measurements of Fabs binding to
their target peptide-HLA complex

| Target | Fab clone | KD (M) | Kon (1/Ms) | Kdis (1/s) | Full R\hat{ }2 |
|---|---|---|---|---|---|
| G2 | G2(1B06) | 4.44E−08 | 1.06E+06 | 3.23E−02 | 0.991 |
| G2 | G2(2A03) | 1.09E−07 | 3.32E+05 | 3.60E−02 | 0.998 |
| G2 | G2(1B12) | 2.28E−08 | 3.66E+05 | 7.28E−03 | 0.980 |
| G2 | G2(2A11) | 2.81E−08 | 6.33E+05 | 1.72E−02 | 0.992 |
| G2 | G2(1H01) | 1.55E−08 | 9.52E+05 | 1.48E−02 | 0.984 |
| G2 | G2(1H11) | 4.99E−08 | 5.81E+05 | 2.80E−02 | 0.994 |
| G7 | G7(2C02) | 5.31E−07 | 1.04E+05 | 5.43E−02 | 0.986 |
| G7 | G7(1A03) | 5.32E−07 | 1.97E+05 | 9.94E−02 | 0.988 |
| G7 | G7(2E09) | 1.18E−08 | 1.85E+05 | 2.12E−03 | 0.992 |

### Example 33: Positional Scanning of G2, G5, G7, G8, and G10 Restricted Peptide Sequences

Positional scanning of the G2, G5, G7, G8, and G10 restricted peptides was carried out to determine the amino acid residues which act as contact points for selected Fab clones or critical residues that impact, directly or indirectly, the interaction of the HLA-PEPTIDE target with the Fab.

FIG. 12 of International Application No. PCT/US2020/1573, which is hereby incorporated by reference in its entirety depicts a general experimental design for the positional scanning experiments. Positional scanning libraries of variant G2, G5, G7, G8, and G10 restricted peptides were generated with amino acid substitutions at a single position in the restricted peptide sequence, scanning across all positions. The amino acid substitutions at a given position were either alanine (conservative substitution), arginine (positively charged), or aspartate (negatively charged). Peptide-HLA complexes comprising the positional scanning library members and the HLA subtype allele were generated as described in Example 31. Stability of the resulting complexes was determined using conditional ligand peptide exchange and stability ELISA as described in Example 31. Such stability analysis may identify residues on the restricted peptide which are important for binding and stabilizing the HLA molecule. Binding affinity of the selected Fab clone to the variant peptide-HLA complexes was assessed by BLI as described in Example 32. Positional variants that result in stable HLA complex formation and weakened Fab binding may identify residues that are likely involved, directly or indirectly, in determining the interaction of the peptide-HLA complex with the Fab clone.

International Application No. PCT/US2020/1573, which is hereby incorporated by reference in its entirety, depicts stability results for the G5 positional variant-HLAs, indicating that the majority of peptide mutations does not impact binding of those peptides to the relevant pHLA.

International Application No. PCT/US2020/1573, which is hereby incorporated by reference in its entirety, depicts binding affinity of Fab clone G5(7A05) to the G5 positional variant-HLAs, indicating positions P2-P8 of the restricted peptide as likely involved, directly or indirectly, in determining the interaction of the peptide-HLA complex with the Fab clone.

International Application No. PCT/US2020/1573, which is hereby incorporated by reference in its entirety, depicts stability results for the G8 positional variant-HLAs, indicating that positions P2, P7 and P10 were not amenable to substitution with the Arg- or Asp-residue and therefore are likely to be important for the peptide to bind the HLA protein.

International Application No. PCT/US2020/1573, which is hereby incorporated by reference in its entirety, depicts binding affinity of Fab clone G8(2C10) to the G8 positional variant-HLAs, indicating positions P1-P5 of the restricted peptide as likely involved, directly or indirectly, in determining the interaction of the peptide-HLA complex with the Fab clone.

International Application No. PCT/US2020/1573, which is hereby incorporated by reference in its entirety, depicts binding affinity of Fab clone G8(1C11) to the G8 positional variant-HLAs, indicating positions P3-P6 of the restricted peptide as likely involved, directly or indirectly, in determining the interaction of the peptide-HLA complex with the Fab clone.

International Application No. PCT/US2020/1573, which is hereby incorporated by reference in its entirety, depicts stability results for the G10 positional variant-HLAs, indicating that positions 2, 5, 8, and 10 were not amenable to amino acid substitution and therefore are likely to be important for the peptide to bind the HLA protein.

International Application No. PCT/US2020/1573, which is hereby incorporated by reference in its entirety, depicts binding affinity of Fab clone G10(1B07) to the G10 positional variant-HLAs, indicating positions P4, P6, and P7 of the restricted peptide as likely involved, directly or indirectly, in determining the interaction of the peptide-HLA complex with the Fab clone.

A map of the amino acid substitutions for the positional scanning experiments for G2 and G7 restricted peptides is shown in International Application No. PCT/US2020/1573, which is hereby incorporated by reference in its entirety. Asterisks denote lack of amino acid substitution.

International Application No. PCT/US2020/1573, which is hereby incorporated by reference in its entirety, depicts stability results for the G2 positional variant-HLAs, indicating that positions 2, 3, and 9 were not amenable to amino acid substitutions and therefore are likely to be important for the peptide to bind the HLA protein.

International Application No. PCT/US2020/1573, which is hereby incorporated by reference in its entirety, depicts binding affinity of Fab clone G2(1H11) to the G2 positional variant-HLAs, indicating positions 3-8 of the restricted peptide as likely involved, directly or indirectly, in determining the interaction of the peptide-HLA complex with the Fab clone.

International Application No. PCT/US2020/1573, which is hereby incorporated by reference in its entirety, depicts stability results for the G7 positional variant-HLAs, indicating that positions 1, 2, 6, and 9 were not amenable to amino acid substitutions and therefore are likely to be important for the peptide to bind the HLA protein.

International Application No. PCT/US2020/1573, which is hereby incorporated by reference in its entirety, depicts binding affinity of Fab clone G7(2E09) to the G7 positional variant-HLAs, indicating positions 1-5 of the restricted peptide as likely involved, directly or indirectly, in determining the interaction of the peptide-HLA complex with the Fab clone.

### Example 34: Antibodies Bind Cells Presenting HLA-PEPTIDE Target Antigens

To verify that the identified TCR-like antibodies bind their pHLA target G2, G5, G7, G8 and G10 in their natural context, e.g., on the surface of antigen-presenting cells, selected clones were reformatted to IgG and used in binding experiments with K562 cells expressing the cognate HLA-PEPTIDE target. Briefly, cells were transduced with either HLA-B*35:01 for the G5 target peptide, HLA-A*02:01 for the G7 and G8 target peptides, or HLA-A*01:01 for the G2 and G10 target peptides. The cells were then exogenously pulsed with target or negative control peptide as specified in Tables 1 and 2, using established methods to generate the relevant pHLA complexes on the cell surface.

Materials and Methods

Retroviral Production

The Phoenix-AMPHO cells (ATCC®, CRL-3213™) were cultured in DMEM (Corning™, 17-205-CV) supplemented with 10% FBS (Seradigm, 97068-091) and Glutamax (Gibco™, 35050079). K-562 cells (ATCC®, CRL-243™) were cultured in IMDM (Gibco™, 31980097) supplemented with 10% FBS. Lipofectamine LTX PLUS (Fisher Scientific, 15338100) contains a Lipofectamine reagent and a PLUS reagent. Opti-MEM (Gibco™, 31985062) was purchased from Fisher Scientific.

Phoenix cells were plated at 5×10⁵ cells/well in a 6 well plate and incubated overnight at 37° C. For the transfection, 10 μg plasmid, 10 μL Plus reagent and 100 μL Opti-MEM were incubated at room temperature for 15 minutes. Simultaneously, 8 μL Lipofectamine was incubated with 92 μL Opti-MEM at room temperature for 15 minutes. These two reactions were combined and incubated again for 15 minutes at room temperature after which 800 μL Opti-MEM was added. The culture media was aspirated from the Phoenix cells and they were washed with 5 mL pre-warmed Opti-MEM. The Opti-MEM was aspirated from the cells and the lipofectamine mixture was added. The cells were incubated for 3 hours at 37° C. and 3 mL complete culture medium was added. The plate was then incubated overnight at 37° C. The media was replaced with Phoenix culture medium and the plate incubated an additional 2 days at 37° C.

The media was collected and filtered through a 0.45 μm filter into a clean 6 well dish. 20 μL Plus reagent was added to each virus suspension and incubated at room temperature for 15 minutes followed by the addition of 8 μL/well of Lipofectamine and another 15 min room temperature incubation.

K562 Cell Line Generation (Retroviral Transduction with HLA)

K562 cells were counted and resuspended to 5E6 cells/mL and 100 μL added to each virus suspension. The 6 well plate was centrifuged at 700 g for 30 minutes and then incubated at 37° C. for 5-6 hours. The cells and virus suspension were then transferred to a T25 flask and 7 mL K562 culture medium was added. The cells were then incubated for three days. The transduced K562 cells were then cultured in medium supplemented with 0.6 μg/mL Puromycin (Invivogen, ant-pr-1) and selection monitored by flow cytometry.

Flow Cytometry Methods:

HLA-transduced K562 cells were pulsed the night before with 50 μM of peptide (Genscript) in IDMEM containing 1% FBS in 6 well plates and incubated under standard tissue culture conditions. Cells were harvested, washed in PBS, and stained with eBioscience Fixable Viability Dye eFluor 450 for 15 minutes at room temperature. Following another wash in PBS+1-2% FBS, cells were resuspended with IgGs at varying concentrations. Cells were incubated with antibodies for 1 hour at 4° C. After another wash, PE-conjugated goat anti-human IgG secondary antibody (Jackson ImmunoResearch) was added at 1:100 to 1:200 for 30 minutes at 4° C. After washing in PBS+1-2% FBS, cells were resuspended in PBS+1-2% FBS and analyzed by flow cytometry. Flow cytometric analysis was performed on the Attune NxT Flow Cytometer (ThermoFisher) using the Attune NxT Software. Data was analyzed using FlowJo.

Results

Four representative examples of antibody binding to either G5-, G8- or G10-presenting K562 cells, as detected by flow cytometry, are shown in International Application No. PCT/US2020/1573, which is hereby incorporated by reference in its entirety. Antibody binding was observed in a dose-dependent manner that was selective for the relevant target peptides.

In another flow cytometry experiment, HLA-transduced K562 cells were pulsed with 50 μM of target or control peptides as listed in Table 1 for G5 and in Table 2 for G8 and G10, and pHLA-specific antibodies were detected by flow cytometry. HLA-transduced K562 cells were pulsed with 50 μM of target or negative control peptides and antibody binding histograms were plotted for G5(7A05) at 20 μg/mL, G8(2C10) at 30 μg/mL, G10(1B07) at 30 μg/mL, and G8(1C11) at 30 μg/mL. Histograms are depicted in International Application No. PCT/US2020/1573, which is hereby incorporated by reference in its entirety.

Results are shown in International Application No. PCT/US2020/1573, which is hereby incorporated by reference in its entirety, for the G2 and G7 transduced cells. Both G2(1H11) and G7(2E09) selectively bound HLA-transduced K562 cells pulsed with the target peptide, as compared to HLA-transduced cells pulsed with the negative control peptides.

### Example 35: Antibodies Bind to Tumor Cell Lines that Express the Target Gene and HLA Subtype

Tumor cell lines were chosen based on expression of the HLA subtype and target gene of interest, as assessed by a publicly available database (TRON http://celllines.tron-mainz.de). The selection of the tumor cell line for cell binding assays is shown in Table 12 below.

TABLE 12
selection of tumor cell lines for cell binding assay

| Cell line | Target expression | HLA type |
|---|---|---|
| LN229 (G5) | MAGEA6 (137.6 RPKM) | B*35:01; B*35:01 |
| | | (26.53 RPKM) |
| BV173 (G8) | FOXE1 (18.1 RPKM) | A*30:01; A*02:01 |
| | | (142.25 RPKM) |
| Colo829 (G10) | MAGEA3 (119.3 RPKM) | A*01:01; A*0101 |
| | MAGEA6 (215.4 RPKM) | (143.7 RPKM) |

The LN229, BV173, and Colo829 tumor cell lines were propagated under standard tissue culture conditions. Flow cytometry was performed as described in Example 34. Cells were incubated with 30 μg/mL or 0 μg/mL antibody followed by PE conjugated anti-human secondary IgG.

Results are depicted in International Application No. PCT/US2020/1573, which is hereby incorporated by reference in its entirety. Panel A shows a histogram plot for G5(7A05) binding to glioblastoma line LN229. Panel B shows a histogram plot for G8(2C10) binding to leukemia line BV173. Panel C shows a histogram plot for G10(1B07) binding to CRC line Colo829.

### Example 36: Identification of Antibodies or Antigen-Binding Fragments Thereof that Bind HLA-PEPTIDE Complexes

Identification of Single-Chain Variable Fragment (scFv) Antibodies Targeting MHC Class I Molecules Presenting Tumor Antigens

Potent and selective single chain antibodies targeting human class I MHC molecules presenting tumor antigens of interest are identified using phage display. Phage libraries are prepared for screening by removing non-specific class I MHC binders. Multiple soluble human peptide-MHC (pMHC) molecules different from the target pMHCs are utilized to pan pre-existing phage libraries to remove scFvs that non-specifically bind class I MHC. To identify scFvs that selectively bind pMHCs of interest, target pMHCs are utilized for at least 1-3 rounds of panning with the prepared phage library. scFv hits identified in the screen are then evaluated against a panel of irrelevant pMHCs to identify scFv leads that bind selectively to the target pMHCs. Lead scFvs are characterized to determine target binding specificity and affinity. Lead scFvs that demonstrate potent and selective binding are converted to full-length IgG monoclonal antibody (mAb) constructs. In addition, the lead scFvs are incorporated into bi-specific mAb constructs and chimeric antigen receptor (CAR) constructs that can be used to generate CAR T-cells. Full-length bi-specifics or scFV-based bi-specifics can be constructed.

Demonstrate Targeting of Human Tumor Cells In Vitro

Immunohistochemistry techniques are utilized to demonstrate specific binding of lead antibodies to human tumor cells or cell lines expressing target pMHC molecules. T-cell lines transfected with CAR-T constructs are incubated with human tumor cells to demonstrate killing of tumor cells in vitro. Alternatively, tumor cells expressing the target are incubated with bi-specific constructs (encoding the ABP and an effector domain) and PBMCs or T cells.

In Vivo Proof-of-Concept

Lead antibody or CAR-T constructs are evaluated in vivo to demonstrate directed tumor killing in humanized mouse tumor models. Lead antibody or CAR-T constructs are evaluated in xenograft tumor models engrafted with human tumors and PBMCs. Anti-tumor activity is measured and compared to control constructs to demonstrate target-specific tumor killing.

Identification of Monoclonal Antibodies (mAbs) that Target MHC Class I Molecules Presenting Tumor Antigens Using Rabbit B Cell Cloning Technologies

Potent and selective mAbs targeting human class I MHC molecules presenting tumor antigens of interest are identified. Soluble human pMHC molecules presenting human tumor antigens are utilized for multiple mouse or rabbit immunizations followed by screening of B cells derived from the immunized animals to identify B cells that express mAbs that bind to target class I MHC molecules. Sequences encoding the mAbs identified from the mouse or rabbit screens will be cloned from the isolated B cells. The recovered mAbs are then evaluated against a panel of irrelevant pMHCs to identify lead mAbs that bind selectively to the target pMHCs. Lead mAbs will be fully characterized to determine target binding affinity and selectivity. Lead mAbs that demonstrate potent and selective binding are humanized to generate full-length human IgG monoclonal antibody (mAb) constructs. In addition, the lead mAbs are incorporated into bi-specific mAb constructs and chimeric antigen receptor (CAR) constructs that can be used to generate CAR T-cells. Full-length bi-specifics or scFV-based bi-specifics can be constructed.

Demonstrate Targeting of Human Tumor Cells In Vitro

Immunohistochemistry techniques are utilized to demonstrate specific binding of lead antibodies to human tumor cells expressing target pMHC molecules. T-cell lines transfected with CAR-T constructs are incubated with human tumor cells to demonstrate killing of tumor cells in vitro. Alternatively, tumor cells expressing the target are incubated with bi-specific constructs (encoding the ABP and an effector domain) and PBMCs or T cells.

In Vivo Proof-of-Concept

Lead antibody or CAR-T constructs are evaluated in vivo to demonstrate directed tumor killing in humanized mouse tumor models. Lead antibody or CAR-T constructs are evaluated in xenograft tumor models engrafted with human PBMCs. Anti-tumor activity is measured and compared to control constructs to demonstrate target-dependent tumor killing.

Potent and selective ABPs that selectively target human class I MHC molecules presenting tumor antigens will be identified using phage display or B cell cloning technologies. The utility of the ABPs will be demonstrated by showing that the ABPs mediated tumor cell killing in vitro and in vivo when incorporated into antibody or CAR-T cell constructs.

### Example 37: Identification of Monoclonal Antibodies (mAbs) that Target MHC Class I Molecules Presenting Tumor Antigens Using Rabbit B Cell Cloning Technologies

Potent and selective mAbs targeting human class I MHC molecules presenting tumor antigens of interest are identified. Soluble human pMHC molecules presenting human tumor antigens are utilized for multiple mouse or rabbit immunizations followed by screening of B cells derived from the immunized animals to identify B cells that express mAbs that bind to target class I MHC molecules. Sequences encoding the mAbs identified from the mouse or rabbit screens will be cloned from the isolated B cells. The recovered mAbs are then evaluated against a panel of irrelevant pMHCs to identify lead mAbs that bind selectively to the target pMHCs. Lead mAbs will be fully characterized to determine target binding affinity and selectivity. Lead mAbs that demonstrate potent and selective binding are humanized to generate full-length human IgG monoclonal antibody (mAb) constructs. In addition, the lead mAbs are incorporated into bi-specific mAb constructs and chimeric antigen receptor (CAR) constructs that can be used to generate CAR T-cells. Full-length bi-specifics or scFV-based bi-specifics can be constructed.

Demonstrate Targeting of Human Tumor Cells In Vitro

Immunohistochemistry techniques are utilized to demonstrate specific binding of lead antibodies to human tumor cells expressing target pMHC molecules. T-cell lines transfected with CAR-T constructs are incubated with human tumor cells to demonstrate killing of tumor cells in vitro. Alternatively, tumor cells expressing the target are incubated with bi-specific constructs (encoding the ABP and an effector domain) and PBMCs or T cells.

In Vivo Proof-of-Concept

Lead antibody or CAR-T constructs are evaluated in vivo to demonstrate directed tumor killing in humanized mouse tumor models. Lead antibody or CAR-T constructs are evaluated in xenograft tumor models engrafted with human PBMCs. Anti-tumor activity is measured and compared to control constructs to demonstrate target-dependent tumor killing.

Potent and selective ABPs that selectively target human class I MHC molecules presenting tumor antigens will be identified using phage display or B cell cloning technologies. The utility of the ABPs will be demonstrated by showing that the ABPs mediated tumor cell killing in vitro and in vivo when incorporated into antibody or CAR-T cell constructs.

### Example 38: Assessment of scFv-pHLA or Fab-pHLA Structures by Hydrogen/Deuterium Exchange and Mass Spectrometry

Experimental Procedures

Hydrogen/Deuterium Exchange.

20 μM of HLA-peptide was incubated with a ˜3-fold molar excess of scFv or Fab formatted proteins for 20 min at room temperature (20-25° C.) to generate complexes for the exchange experiments. For the Apo (unbound) control, the HLA-peptide was incubated with an equal volume of 50 mM NaCl, 20 mM Tris pH 8.0. All subsequent reaction steps were performed at 4° C. by an automated HDX PAL system controlled by Chronos 4.8.0 software (Leap Technologies, Morrisville, NC). 5 μl of protein complexes were diluted 10-fold into H2O or 50 mM NaCl, 20 mM Tris pH 8.0 (for the 0 min. control time-point) or the same buffer made with D20 for 30 s prior to quenching in 0.8 M guanidine hydrochloride, 0.4% acetic acid (v/v), and 75 mM tris(2-carboxyethyl) phosphine for 3 min. ˜50 μmol of quenched protein complexes were transferred onto an immobilized Protein XIII/Pepsin column (NovaBioAssays, Woburn, MA) for integrated on-line protein digestion.

Liquid Chromatography, Mass Spectrometry, and HDX Analysis

Chromatographic separation of peptides was carried out using an UltiMate 3000 Basic Manual UHPLC System (ThermoFisher Scientific, Waltham, MA), which contained a trap C18 column (5 μM particle size and 2.1 mm diameter) and an analytical C18 column (1.9 μM particle size and 1 mm diameter). Samples were desalted with 10% acetonitrile, 0.05% trifluoroacetic acid or 10% acetonitrile, 0.5% formic acid at a 40 μl/min flow rate for 2 min and peptides were eluted at a 40 μl/min flow rate with an increasing concentration gradient of 95% acetonitrile with trifluoro acetic acid or formic acid. Mass spectrometry was performed with an Orbitrap Fusion Lumos mass spectrometer (ThermoFisher, Waltham, MA) with the ESI source set at a positive ion voltage of 3500-3800 V. Prior to performing hydrogen-deuterium exchange experiments, peptide fragments of each HLA-peptide complex were analyzed by data-dependent LC/MS/MS and the data searched using PEAKS Studio (Bioinformatics Solutions Inc., Waterloo, ON, Canada) with a peptide precursor mass tolerance of 20 ppm and fragment ion mass tolerance of 0.2 Da. The HLA, 02M, and target peptide sequences were searched, and false detection rates identified using a decoy-database strategy. Peptides from the hydrogen-deuterium experiments were detected by LC/MS and analyzed by HDX Workbench (Omics Informatics, Honolulu, HI) with a retention time window size of 0.22 min and a 7.0 ppm error tolerance. High-resolution HD exchange data for selected peptides were obtained by fragmenting the peptides by Electron Transfer Dissociation (ETD) with a reaction time of 200 ms (G2) or 100 ms (G10), using fluoranthene as the reagent anion. Peptide fragments were analyzed by HDExaminer (Sierra Analytics) with a retention time window size of 18 s and a peptide m/z tolerance of 2 Da. Heat maps of deuterium uptake differences were generated by Microsoft Excel and mapped on to relevant protein crystallographic structures using Pymol (Schrödinger, Cambridge, MA).

Results

The results shows an exemplary heatmap of the HLA portion of the G8 HLA-PEPTIDE complex when incubated with scFv clone G8(1H08), visualized in its entirety using a consolidated perturbation view. (International Application No. PCT/US2020/1573, which is hereby incorporated by reference in its entirety).

An example of the data from scFv G8(1H08) plotted on the crystal structure described in Example 39 is shown International Application No. PCT/US2020/1573, which is hereby incorporated by reference in its entirety.

International Application No. PCT/US2020/1573, which is hereby incorporated by reference in its entirety, shows an exemplary heatmap of the HLA portion of the G8 HLA-PEPTIDE complex when incubated with scFv clone G8(1C11), visualized in its entirety using a consolidated perturbation view.

An example of the data from scFv G8(1C11) plotted on the crystal structure described in Example 39 is shown in International Application No. PCT/US2020/1573, which is hereby incorporated by reference in its entirety.

International Application No. PCT/US2020/1573, which is hereby incorporated by reference in its entirety, shows an exemplary heatmap of the HLA portion of the G10 HLA-PEPTIDE complex when incubated with scFv clone G10(2G11), visualized in its entirety using a consolidated perturbation view.

An example of the data from scFv G10(2G11) plotted on a crystal structure PDB5bs0 is shown in International Application No. PCT/US2020/1573, which is hereby incorporated by reference in its entirety. The crystal structure, depicting a restricted peptide in the HLA binding cleft formed by the α1 and α2 helices, can be found at URL https://www.rcsb.org/structure/5bs0 (Raman et al).

An example of data from a second round of HDX studies, from scFv-G10-P5A08, plotted on a crystal structure 5bs0.pdb is shown in International Application No. PCT/US2020/1573, which is hereby incorporated by reference in its entirety. The crystal structure, depicting a restricted peptide in the HLA binding cleft formed by the α1 and α2 helices, can be found at URL https://www.rcsb.org/structure/5bs0 (Raman et al).

To better compare the data across the ABPs tested for a given HLA-PEPTIDE target, data for each ABP was exported, and a heat map was generated in Excel. International Application No. PCT/US2020/1573, which is hereby incorporated by reference in its entirety shows resulting heat maps across the HLA α1 helix for all ABPs tested for HLA-PEPTIDE target G8 (HLA-A*02:01_AIFPGAVPAA (SEQ ID NO: 6)). International Application No. PCT/US2020/1573, which is hereby incorporated by reference in its entirety shows resulting heat maps across the HLA α2 helix for all ABPs tested for HLA-PEPTIDE target G8 (HLA-A*02:01_AIFPGAVPAA (SEQ ID NO: 6). International Application No. PCT/US2020/1573, which is hereby incorporated by reference in its entirety shows resulting heat maps across the restricted peptide AIFPGAVPAA (SEQ ID NO: 6) for all ABPs tested. The heat maps indicate positions 45-60 of the HLA protein (in the α1 helix) of HLA-PEPTIDE target G8 (HLA-A*02:01_AIFPGAVPAA (SEQ ID NO: 6)) as likely involved, directly or indirectly, in determining the interaction between the HLA-PEPTIDE target and G8-specific antibody-based ABPs.

International Application No. PCT/US2020/1573, which is hereby incorporated by reference in its entirety, shows resulting heat maps from a first round of HDX experiments across the HLA α1 helix for all ABPs tested for HLA-PEPTIDE target G10 (HLA-A*01:01_ASSLPTTMNY (SEQ ID NO: 7)). International Application No. PCT/US2020/1573, which is hereby incorporated by reference in its entirety, shows resulting heat maps from a first round of HDX experiments across the HLA α2 helix for all ABPs tested for HLA-PEPTIDE target G10 (HLA-A*01:01_ASSLPTTMNY (SEQ ID NO: 7)). International Application No. PCT/US2020/1573, which is hereby incorporated by reference in its entirety, shows resulting heat maps from a first round of HDX experiments across the restricted peptide ASSLPTTMNY (SEQ ID NO: 7) for all ABPs tested. International Application No. PCT/US2020/1573, which is hereby incorporated by reference in its entirety, shows resulting heat maps from a second round of HDX experiments across the HLA α1 helix, the HLA α2 helix, and the restricted peptide ASSLPTTMNY (SEQ ID NO: 7) for all ABPs tested. Taken together, the heat maps indicate positions 49-56 and/or 59-66 of the HLA protein (in the α1 helix), as well as positions 136-147 and 157-160 of the α2 helix of the HLA protein, as likely involved, directly or indirectly, in determining the interaction between the HLA-PEPTIDE target and G10-specific antibody-based ABPs. In particular, all of the ABPs tested decreased solvent accessibility of positions 52-54 of the HLA α1 helix.

An example of the data from scFv G2(1G07) plotted on a crystal structure PDB 5bs0 is shown in International Application No. PCT/US2020/1573, which is hereby incorporated by reference in its entirety. The crystal structure can be found at URL https://www.rcsb.org/structure/5bs0 (Raman et al). Areas not covered with MS data are shown in black and those with the greatest decrease in D exchange (indicating a binding site for the ABP) is circled. For clarity, only the binding groove and helices are shown.

An exemplary heatmap for scFv clone G2(1G07) visualized in its entirety using a consolidated perturbation view is shown in International Application No. PCT/US2020/1573, which is hereby incorporated by reference in its entirety.

An example of the data from scFv G2(2C11)plotted on a crystal structure PDB 5bs0 is shown in International Application No. PCT/US2020/1573, which is hereby incorporated by reference in its entirety.

International Application No. PCT/US2020/1573, which is hereby incorporated by reference in its entirety, shows high resolution HDX data plotted on a crystal structure PDB 5bs0. Data for G2 bound to four different scFvs were obtained by fragmenting peptides by Electron Transfer Dissociation (ETD) as described in the Experimental Procedures.

To better compare the data across the ABPs tested for a given HLA-PEPTIDE target, data for each ABP was exported, and a heat map was generated in Excel. Resulting heat maps are shown in International Application No. PCT/US2020/1573, which is hereby incorporated by reference in its entirety, showing a heat map across the α1 helix (top) and across the α2 helix (bottom). The figures shows a heat map for all ABPs tested for A*01:01_NTDNNLAVY (SEQ ID NO: 5), across restricted peptide residues 1-9. Heat maps from a second round of HDX data are shown in International Application No. PCT/US2020/1573, which is hereby incorporated by reference in its entirety. Taken together, the heat maps elucidated regions of reduced solvent accessibility in the HLA alpha subunits that bind and display the target peptide. Many of these regions were shared across multiple A*01:01_NTDNNLAVY (SEQ ID NO: 5) specific ABPs. The two regions which most commonly exhibited decreased solvent accessibility include A70-Y85 of the alpha 1 helix, and/or positions A140-Y160 of the alpha 2 helix, with all ABPs shielding R157-Y160 of the helix. Taken together, the heat maps also indicate HLA-PEPTIDE/ABP interactions that decrease solvent accessibility across positions 3-9 of the restricted peptide. The effect was increasingly pronounced towards the C-terminal direction. This pattern was consistent for 14 of the 15 antibodies examined, with positions 6-9 invariably being shielded by presence of the ABPs. Furthermore, the heat maps indicate that HLA residues 157-160 (RRVY (SEQ ID NO: 440)) are important contact points of the A*01:01_NTDNNLAVY (SEQ ID NO: 5) HLA-PEPTIDE target complex for binding to its specific ABP. All clone entries in the HDX heat maps are scFv formats unless otherwise noted.

International Application No. PCT/US2020/1573, which is hereby incorporated by reference in its entirety shows an example of high resolution data from scFv clone G5-P1C12 plotted on crystal structure of HLA-B*35:01 (5xos.pdb; https://www.rcsb.org/structure/5XOS).

International Application No. PCT/US2020/1573, which is hereby incorporated by reference in its entirety shows resulting color heat maps from high resolution HDX experiments across the HLA α1 helix, the HLA α2 helix, and restricted peptide EVDPIGHVY (SEQ ID NO: 9) for all ABPs tested for HLA-PEPTIDE target G5 (HLA-B*35:01_EVDPIGHVY (SEQ ID NO: 9)). The figures shows a numerical representation of the color heat map. These heat maps indicate positions 50, 54, 55, 57, 61, 62, 74, 81, 82 and 85 of the HLA protein (in the α1 helix) as likely involved, directly or indirectly, in determining the interaction between the HLA-PEPTIDE target and G5-specific antibody-based ABPs. These heat maps indicate positions 147 and 148 of the HLA protein (in the α2 helix) as likely involved, directly or indirectly, in determining the interaction between the HLA-PEPTIDE target and G5-specific antibody-based ABPs.

An example of high-resolution HDX data from scFv G8-P1H08 plotted on a crystal structure of Fab clone G8-P1C11 complexed with HLA-PEPTIDE target A*02:01_AIFPGAVPAA (SEQ ID NO: 6) (“G8”), is shown in International Application No. PCT/US2020/1573, which is hereby incorporated by reference in its entirety.

International Application No. PCT/US2020/1573, which is hereby incorporated by reference in its entirety, shows resulting color heat maps from high resolution HDX experiments across the HLA α1 helix, the HLA α2 helix, and restricted peptide AIFPGAVPAA (SEQ ID NO: 6) for all ABPs tested for HLA-PEPTIDE target G8 (HLA-A*02:01_AIFPGAVPAA (SEQ ID NO: 6)). International Application No. PCT/US2020/1573, which is hereby incorporated by reference in its entirety shows a numerical representation of the color heat maps. The heat maps from the second round of HDX data indicate positions 46, 49, 55, 61, 74, 76, 77, 78, 81 and 84 of the HLA protein (in the α1 helix) as likely involved, directly or indirectly, in determining the interaction between the HLA-PEPTIDE target and G8-specific antibody-based ABPs. The heat maps from the second round of HDX data indicate positions 137, 138, 145, 147, 152-157 of the HLA protein (in the α2 helix) as likely involved, directly or indirectly, in determining the interaction between the HLA-PEPTIDE target and G8-specific antibody-based ABPs. The heat maps from the second round of HDX data indicate positions 5 and 6 of the restricted peptide AIFPGAVPAA (SEQ ID NO: 6) as likely involved, directly or indirectly, in determining the interaction between the HLA-PEPTIDE target and G8-specific antibody-based ABPs.

### Example 39: Assessment of Fab-pHLA Structures by Crystallography

Materials and Methods

Complex Purification and Crystal Screening

Fab fragments corresponding to, e.g., HLA-PEPTIDE target G8 (A*02:01_AIFPGAVPAA (SEQ ID NO: 6)) were concentrated to reach 5 mg/mL (100 PM) before addition of its corresponding HLA-MHC (1:1 molar ratio) and incubated for 30 minutes at 4° C. The mixture was then injected on size exclusion chromatography column (S200 16/60) equilibrated in 1×PBS buffer for complex purification. Fractions containing both Fab and HLA and with an elution volume coherent with a complex of ˜94 kDa were pooled and concentrated to 10-12 mg/mL (1 AU=1 mg/mL) Each purified complex was screened for crystallization conditions using commercial screens: PEGIon (Hampton research), JCSG+ (Molecular Dimensions) and JBS Screen 3 and 4 (Jena Biosciences). The choice of the kits was driven by the characteristic of known crystal conditions of HLA-Fab complexes that are mainly based on the use of PEG3350 or PEG4000 as precipitant. 3 to 4 weeks after screen, diffraction suitable crystals appeared for HLA-Fab combinations in several crystallization conditions (Table 18). The protein nature of the crystals was checked by UV. Crystals were transferred into a cryoprotectant solution (crystallization solution supplemented with 25% Glycerol) and flash frozen in liquid nitrogen.

Data Collection and Processing

Diffraction data was collected on the Proxima 2A beamline at SOLEIL synchrotron (Gif sur Yvette, France). Data processing and scaling was performed using XDS (1). Molecular replacement was performed using MolRep and Arp/Warp from the CCP4 suite (2) using PDB 5E6I for HLA (100% sequence identity) and 5AZE (90% sequence identity with VH) and 5115 (97% sequence identity with VL) for Fab as entry models. Refinement was performed using Buster TNT (GlobalPhasing, Inc) and manual model modifications in Coot (CCP4 suite).

Complex Purification

Combinations produced a good separation between the individual protein peak and the formed complex peak (see International Application No. PCT/US2020/1573, which is hereby incorporated by reference in its entirety). Increasing incubation time to 16 hours (overnight) did not change the ratio of complex formed (˜50% of the protein is present in complex and 50 as free proteins). Peak analysis by SDS PAGE under reducing conditions showed the presence of both Fab chains (30 kDa), HLA heavy chain (˜35 kDa), and LA light chain (BLM, <10 kDa) in the pooled fractions (see International Application No. PCT/US2020/1573, which is hereby incorporated by reference in its entirety).

Crystallization and Data Collection

Complex pooled fractions were concentrated and screened. After 3-4 weeks crystals appeared for some of the HLA-Fab combinations. A summary of the crystallography conditions for the A*02:01_AIFPGAVPAA (SEQ ID NO: 6)-G8(1C11) Fab complex and resulting crystal formation is shown in Table 18.

TABLE 18
Crystallography conditions

| | | Crystals |
|---|---|---|
| Commercial | | Obtained |
| Kit | Experimental Conditions | (Y/N) |
| JBS | 20% PEG4000, 200 mM Magnesium sulfate, | No |
| | 10% glycerol (GOL) | |
| JBS | 20% PEG4000, 200 mM Magnesium sulfate, | Yes |
| | 5% 2-Propanol | |
| JBS | 20% w/v Polyethylene glycol 4,000 10% w/v | No |
| | 2-Propanol, 100 mM HEPES; pH 7.5 | |
| JCSG | 20% (w/v) PEG 3350 200 mM Ammonium | No |
| | chloride | |
| JCSG | 30% (w/v) PEG 2000 MME 100 mM Potassium | No |
| | thiocyanate | |
| JCSG | 25% (w/v) PEG 3350 100 mM Bis-Tris/ | Yes |
| | Hydrochloric acid pH 5.5 | (integrated |
| | | into P1 |
| | | Space |
| | | group) |
| JCSG | 30% v/v Jeffamine ® M-600, 0.1M HEPES pH | Yes |
| | 7.0 | |
| JCSG | 25% (w/v) PEG 3350 100 mM Bis-Tris/ | No |
| | Hydrochloric acid pH 5.5, 200 mM Lithium | |
| | sulfate | |
| PEGion | 0.2 M Ammonium tartrate dibasic pH 7.0, 20% | Yes |
| | w/v Polyethylene glycol 3,350 | (integrated |
| | | into P1 |
| | | Space |
| | | group) |
| PEGion | 2% v/v TacsimateTM pH 6.0 0.1M BIS-TRIS | No |
| | pH 6.5 20% PEG3350 | |
| PEGion | 1% w/v Tryptone 0.001M Sodium azide, 0.05M | No |
| | HEPES sodium pH 7.0, 20% w/v | |
| | Polyethylene glycol 3,350 | |

Out of the tested conditions, four yielded crystals. Two yielded crystals which diffracted well (1.7 to 2.0 Å resolution) and were integrated into a P1 space group (Table 18). Structure resolution was possible by combining molecular replacement (MolRep) and software automated model building using Arp/Warp.

An exemplary crystal of a complex comprising Fab clone G8(1C11) and HLA-PEPTIDE target A*02:01_AIFPGAVPAA (SEQ ID NO: 6) (“G8”) is shown in International Application No. PCT/US2020/1573, which is hereby incorporated by reference in its entirety. This crystal was grown using the commercial screen JCSG, using 25% (w/v) PEG 3350 100 mM Bis-Tris/Hydrochloric acid pH 5.5. This crystal was used to generate the structural data below.

Structural Analysis

The overall structure of a complex formed by binding of Fab clone G8(1C11) to HLA-PEPTIDE target A*02:01_AIFPGAVPAA (SEQ ID NO: 6) (“G8”) is shown in International Application No. PCT/US2020/1573, which is hereby incorporated by reference in its entirety. The individual proteins are represented as surfaces. The interface area between the HLA and the VH and VL is 747 Å² and 285 Å², respectively.

During refinement electron density region corresponding to the peptide was clearly visible and allowed peptide side chain unambiguous positioning (see International Application No. PCT/US2020/1573, which is hereby incorporated by reference in its entirety) with the provided 10 residue peptide sequence AIFPGAVPAA (SEQ ID NO: 6). All areas relevant to interaction interfaces are refined; however, some refinement is still required in antibody constant regions.

Coding of monomers in the complex, which is referred to in the following data, is provided in Table 19 below.

TABLE 19
monomer coding used in crystal analysis

| Monomer | Monomer Code (ID) |
|---|---|
| HLA heavy chain (α1, α2, α3) | A |
| HLA β2 microglobulin (light chain) | B |
| Restricted peptide | I |
| Fab heavy chain (VH-CH1) | C |
| Fab light chain (VL-CL) | D |

HLA-Peptide Interaction

The restricted peptide AIFPGAVPAA (SEQ ID NO: 6) is mainly buried in the HLA A*02:01 binding pocket with the residues P4G5A6 protruding towards the Fab. The interaction surface between the peptide and the HLA is 926 Å² and represents 76% of the total peptide solvent accessible surface (1215 Å²). The binding of the peptide to the HLA involves 9 hydrogen bonds and van der Waals interactions (see International Application No. PCT/US2020/1573, which is hereby incorporated by reference in its entirety) and yields a binding energy of −16.4 kcal/mol.

TABLE 20
Hydrogen bond interactions between restricted peptide and HLA.

| | Distance | |
|---|---|---|
| Peptide | (Angstroms) | HLA |
| I:ALA 1[N] | 2.72 | A:TYR 172[OH] |
| I:ALA 1[N] | 2.86 | A:TYR 8[OH] |
| I:ILE 2[N] | 2.81 | A:GLU 64[OE1] |
| I:ILE 2[N] | 3.71 | A:TYR 8[OH] |
| I:PHE 3[N] | 2.94 | A:TYR 100[OH] |
| I:ALA 1[O] | 2.67 ] | A:TYR 160[OH |
| I:PRO 8[O] | 2.93 | A:ARG 98[NH2] |
| I:PRO 8[O] | 2.89 | A: ARG 98[NH1] |
| I:ALA 9[O] | 2.71 | A:TRP 148[NE1] |
| I:ALA 1[N] | 2.72 | A:TYR 172[OH] |

A complete interface summary of the HLA and restricted peptide is shown in International Application No. PCT/US2020/1573, which is hereby incorporated by reference in its entirety.

A complete list of the interacting residues from the restricted peptide and HLA is shown in International Application No. PCT/US2020/1573, which is hereby incorporated by reference in its entirety.

Fab-Restricted Peptide Interactions

As most of the peptide is buried in the binding pocket of the HLA, only part of it available for interactions with the Fab chains. This is confirmed by the observation that 76% of the solvent accessible area of the peptide is occupied by its interaction with the HLA. Interaction surface between the peptide and the heavy chain and the light chain of the Fab is 114.3 and 113.9 Å² respectively. This corresponds to 18% of the total peptide solvent accessible area. PISA analysis showed that only two hydrogen bonds are involved in the interaction between the Fab and the peptide: hydroxyl group of Tyr32 from the light chain interacts with the backbone carbonyl of Gly5 of the peptide and the Tyr100A backbone amide interacting with the backbone carbonyl group of Pro4 of the peptide (See Table 21 for a list of the hydrogen interactions, below).

TABLE 21
Fab/restricted peptide H bond interactions

| Peptide | Distance (A) | Fab |
|---|---|---|
| I:PRO 4[O] | 3.0 | C:TYR 100A[OH] |
| | | (VH) |
| I:GLY 5[O] | 3.7 | D:TRY 32[OH] (VL) |

The recognition mode of the Fab towards the restricted peptide is mainly through hydrophobic interactions and hydrogen bonds involving solvent molecules (see International Application No. PCT/US2020/1573, which is hereby incorporated by reference in its entirety). The binding energy of the interaction between the Fab and restricted peptide is −2.0 and −1.9 kcal/mol with the VH and VL chains respectively.

A complete interface summary of the Fab VH chain and restricted peptide, and a complete list of the interacting residues from the Fab VH chain and restricted peptide, is shown in International Application No. PCT/US2020/1573, which is hereby incorporated by reference in its entirety.

A complete interface summary of the Fab VL chain and restricted peptide, and a complete list of the interacting residues from the Fab VL chain and restricted peptide, is shown in International Application No. PCT/US2020/1573, which is hereby incorporated by reference in its entirety.

Fab-HLA Interactions

The Fab and the HLA moieties interacts extensively as shown by interface area between the HLA and the Fab with a total of 1032 Å². The interaction between the HLA and the VH chain is composed of hydrophobic interactions, 6 H bonds and 3 salt bridges (see International Application No. PCT/US2020/1573, which is hereby incorporated by reference in its entirety). This interaction represents the major interaction are with 747 Å² (72% of the total contact area).

A table of the hydrogen bond contacts between the VH chain of the Fab and the HLA protein is shown below.

TABLE 22
hydrogen bond contacts between VH and HLA.

| Fab VH | Distance | HLA |
|---|---|---|
| C:SER 31[OG] | 2.71 | A:THR 164[OG1] |
| C:TYR 100A[OH] | 2.55 | A:THR 164[OG1] |
| C:SER 31[N] | 3.17 | A:GLU 167[OE1] |
| C:SER 30[N] | 2.86 | A:GLU 167[OE2] |
| C:TYR 32[OH] | 2.80 | A:LYS 67[NZ] |
| C:TYR 98[O] | 2.94 | A:ARG 66[NH2] |
| C:ASP 100[OD1] | 2.88 | A:ARG 66[NH1] |

A table of the salt bridge contacts between the VH chain of the Fab and the HLA protein is shown below.

TABLE 23
salt bridge contacts between VH and HLA.

| Fab VH | Distance | HLA |
|---|---|---|
| C:ASP 100[OD1] | 2.88 | A:ARG 66[NH1] |
| C:ASP 100[OD1] | 3.39 | A:ARG 66[NH2] |
| C:ASP 100[OD2] | 3.40 | A:ARG 66[NH1] |

A complete interface summary of the Fab VH chain and HLA protein is shown in International Application No. PCT/US2020/1573, which is hereby incorporated by reference in its entirety.

A complete list of the interacting residues from the Fab VH chain and HLA protein is shown in International Application No. PCT/US2020/1573, which is hereby incorporated by reference in its entirety.

A table of the hydrogen bond contacts between the VL chain of the Fab and the HLA protein is shown in Table 24 below.

TABLE 24
hydrogen bonds between VL and HLA.

| Fab VL | Distance | HLA |
|---|---|---|
| D:ILE 94[N] | 3.56 | A:ALA 151[O] |
| D:SER 30[OG] | 2.84 | A:GLN 73[NE2] |
| D:ILE 94 [O] | 3.00 | A:HIS 152[ND1] |

A complete interface summary of the Fab VL chain and HLA protein is shown in International Application No. PCT/US2020/1573, which is hereby incorporated by reference in its entirety.

A complete list of the interacting residues from the Fab VL chain and HLA protein is shown in International Application No. PCT/US2020/1573, which is hereby incorporated by reference in its entirety.

### Example 40: Identification of Predicted HLA-PEPTIDE Complexes (Table A1)

We identified cancer specific HLA-peptide targets using three computational steps: First, we identified genes that are not generally expressed in most normal tissues using data available through the Genotype-Tissue Expression (GTEx) Project [1]. We then identified which of those genes are aberrantly expressed in cancer samples using data from The Cancer Genome Atlas (TCGA) Research Network: http://cancergenome.nih.gov/. In these genes, we identified which peptides are likely to be presented as cell surface antigens by MHC Class I proteins using a deep learning model trained on HLA presented peptides sequenced by MS/MS, as described in international patent application no. PCT/US2016/067159, herein incorporated by reference, in its entirety, for all purposes.

To identify genes that are not usually expressed in normal tissues, we obtained aggregated gene expression data from the Genotype-Tissue Expression (GTEx) Project (version V6p). This dataset comprised 8,555 post-mortem samples from over 50 tissue types. Expression was measured using RNA-Seq and computationally processed according to the GTEx standard pipeline (https://www.gtexportal.org/home/documentationPage). For the purposes of this analysis, genes were considered not expressed in normal tissues if they were found not to be expressed in any tissues in GTEx or were only expressed in one or more of testis, minor salivary gland, and the endocervix (i.e., immune privileged or non-essential tissues). We also restricted our search to only include protein coding genes. Because GTEx and TCGA use different annotations of the human genome in their computational analyses, we excluded genes which we could not map between the two datasets using standard techniques such as ENCODE mappings.

We sought to define criteria to excluded genes that were expressed in normal tissue that was strict to ensure tumor specificity, but would not exclude non-zero measurements arising from sporadic, low level transcription or potential artifacts such as read misalignment. Therefore, we designated a gene to be not normally expressed in a non-immune privileged or essential tissue if its median expression across GTEx samples was less than 0.5 RPKM (Reads Per Kilobase of transcript per Million mapped reads), and it was never expressed with greater than 10 RPKM, and it was expressed at 5 RPKM in no more than two samples across all essential tissue samples. To exclude genes which were potentially expressed but could not be measured by RNA-Seq using the GTEX analysis pipeline, we also excluded genes which were measured at 0 RPKM in all samples. These criteria left us with a set of protein coding genes that did not appear to be expressed in most normal tissues.

We next sought to identify which of these genes are aberrantly expressed in tumors. We examined 11,093 samples available from TCGA (Data Release 6.0). We considered a gene expressed if it was observed at expression of at least 5 FPKM (Fragments Per Kilobase of transcript per Million mapped reads) in at least 5 samples. Because one fragment usually consists of two mapped reads, 5 FPKM equals approximately 10 RPKM.

While the GTEx data spans a broad range of tissue types, it does not include all cell types that are present in the human body. We therefore further examined the list for the gene's biological function category using the DAVID v 6.8 [2] and used this analysis, along with literature review, to filter the gene list further. We removed genes likely to be expressed in immune cells (e.g., interferon family genes), eye-related genes (e.g., retina in the FANTOM5 dataset http://www.proteinatlas.org), genes expressed in the mouth and nose (e.g. olfactory genes and taste receptors), and genes related to the circadian cycle. We also excluded genes that are part of large gene families, including histone genes, because their expression is difficult to accurately assess with RNA Sequencing due to sequence homology.

We then examined the distribution of the expression of the remaining genes across the TCGA samples. When we examined the known Cancer Testis Antigens (CTAs), e.g., the MAGE family of genes, we observed that the expression of these genes in log space was generally characterized by a bimodal distribution across samples in the TCGA. This distribution included a left mode around a lower expression value and a right mode (or thick tail) at a higher expression level. This expression pattern is consistent with a biological model in which some minimal expression is detected at baseline in all samples and higher expression of the gene is observed in a subset of tumors experiencing epigenetic dysregulation. We reviewed the distribution of expression of each gene across TCGA samples and discarded those where we observed only a unimodal distribution with no significant right-hand tail, as this distribution may (as a non-limiting example) more likely characterize genes that have a low baseline of expression in normal tissues.

This left us with a remaining gene list of >630 genes that was highly enriched for genes involved in testis-specific biological processes and development. Because many of these genes produce different isoforms, these genes mapped to >1,200 proteins using the UNIPROT mapping service. In addition to the genes that met our strict computational criteria, we added several genes that have previously been identified in the scientific literature as cancer testes antigens.

To identify the peptides that are likely to be presented as cell surface antigens by MHC Class I proteins, we used a sliding window to parse each of these proteins into its constituent 8-11 amino acid sequences. We processed these peptides and their flanking sequences with the HLA peptide presentation deep learning model to calculate the likelihood of presentation of each peptide at expression levels between five TPM, which approximately corresponds to one transcript per cell [3], to 200 TPM (i.e., a high level of expression). We considered a peptide a putative HLA-PEPTIDE target if its probability of presentation calculated by our model was greater than 0.1 in 10 or more patients in the TCGA dataset with expression 5 TPM or greater.

The results are shown in Table A1 of International Application No. PCT/US2020/15736 or U.S. application Ser. No. 17/426,627, each of which is hereby incorporated by reference in its entirety. From this example, there are >1,800 HLA-PEPTIDE targets across ˜400 genes and 25 analyzed HLA alleles. For clarity, each HLA-PEPTIDE was assigned a target number in Table A1. For example, HLA-PEPTIDE target 1 is HLA-A*01:01_EVDPIGHLY (SEQ ID NO: 109), HLA-PEPTIDE target 2 is HLA-A*29:02 FVQENYLEY (SEQ ID NO: 441), and so forth.

Collectively, this list of HLA-PEPTIDE targets is expected to be a significant contribution to the state of knowledge of cancer specific targets. In summary, the example provides a large set of tumor-specific HLA-PEPTIDEs that can be pursued as candidate targets for ABP research and development.

### REFERENCES

1. Consortium, G. T., The Genotype-Tissue Expression (GTEx) project. Nat Genet, 2013. 45(6): p. 580-5.
2. Huang da, W., B. T. Sherman, and R. A. Lempicki, Systematic and integrative analysis of large gene lists using DAVID bioinformatics resources. Nat Protoc, 2009. 4(1): p. 44-57.
3. Shapiro, E., T. Biezuner, and S. Linnarsson, Single-cell sequencing-based technologies will revolutionize whole-organism science. Nat Rev Genet, 2013. 14(9): p. 618-30.

### Example 41: Initial Validation of Predicted HLA-PEPTIDE Complexes

As an initial assessment to validate the predicted HLA-PEPTIDE targets arising from the above described approach, we evaluated public databases and selected literature for reports of these targets as having been previously identified by various assay techniques, including HLA binding affinity measurements, HLA peptide mass-spectrometry, as well as measures of T cell responses. Two comprehensive databases containing assay result annotations for HLA-PEPTIDE pairs were used: IEDB (Vita et al., 2015) and T antigen (Olsen et al., 2017). We determined that 19 (15 unique across genes) of the computationally predicted targets were previously reported in the databases, many in genes (e.g., cancer testis antigens) that have long been the subject of study in cancer immunology. See Table B.

TABLE B

| Protein | | Found in IEDB | IEDB | Tantigen |
|---|---|---|---|---|
| Name | HLA-PEPTIDE | or Tantigen | Status | Status |
| MAGEA3 | HLA-A*01:01_EVDPIGHLY | TRUE | Found | Found |
| | (SEQ ID NO: 109) | | | |
| MAGEA3 | HLA-A*29:02_FVQENYLEY | TRUE | Found | Not found |
| | (SEQ ID NO: 441) | | | |
| MAGEA3 | HLA-A*29:02_LVHFLLLKY | TRUE | Found | Not found |
| | (SEQ ID NO: 442) | | | |
| MAGEA3 | HLA-B*44:03_MEVDPIGHLY | TRUE | Not found | Found |
| | (SEQ ID NO: 443) | | | |
| MAGEA6 | HLA-A*29:02_FVQENYLEY | TRUE | Found | Not found |
| | (SEQ ID NO: 441) | | | |
| MAGEA6 | HLA-A*29:02_LVHFLLLKY | TRUE | Found | Not found |
| | (SEQ ID NO: 442) | | | |
| MAGEA4 | HLA-A*01:01_EVDPASNTY | TRUE | Not found | Found |
| | (SEQ ID NO: 444) | | | |
| MAGEA1 | HLA-A*02:01_KVLEYVIKV | TRUE | Found | Found |
| | (SEQ ID NO: 445) | | | |
| MAGEAC | HLA-A*29:02_LVHFLLLKY | TRUE | Found | Not found |
| | (SEQ ID NO: 442) | | | |
| MAGEAC | HLA-A*29:02_LVQENYLEY | TRUE | Found | Not found |
| | (SEQ ID NO: 446) | | | |
| SSX1 | HLA-C*04:01_AFDDIATYF | TRUE | Found | Not found |
| | (SEQ ID NO: 447) | | | |
| MAGEA4 | HLA-A*29:02_WVQENYLEY | TRUE | Found | Not found |
| | (SEQ ID NO: 448) | | | |
| MAGB2 | HLA-A*02:01_GVYDGEEHSY | TRUE | Found | Not found |
| | (SEQ ID NO: 111) | | | |
| MAGEA1 | HLA-A*03:01_SLFRAVITK | TRUE | Found | Found |
| | (SEQ ID NO: 449) | | | |
| MAGEA4 | HLA-A*11:01_ALAETSYVK | TRUE | Found | Not found |
| | (SEQ ID NO: 450) | | | |
| SAGE1 | HLA-A*24:02_LYATVIHDI | TRUE | Not found | Found |
| | (SEQ ID NO: 451) | | | |
| PASD1 | HLA-A*02:01_QLLDGFMITL | TRUE | Found | Not found |
| | (SEQ ID NO: 452) | | | |
| MAGEA8 | HLA-A*29:02_WVQENYLEY | TRUE | Found | Not found |
| | (SEQ ID NO: 448) | | | |
| MAGEAC | HLA-A*29:02_STLPTTINY | TRUE | Found | Not found |
| | (SEQ ID NO: 453) | | | |

Additional limited literature review was carried out for peptides not found in the above public databases. The following peptides were identified, as shown in Table C:

TABLE C

| | | HLA/peptide known | HLA/peptide known status in |
|---|---|---|---|
| HLA allele/ | Protein | status IEDB or | literature (preliminary) if |
| peptide complex | Name | Tantig 2017 | not in IEDB or Tantigen |
| HLA-A*01:01_NTDNNLAVY | KKLC1 | Not known | WO 2017/089756 A1 |
| (SEQ ID NO: 5) | | | (Stevanović et al., 2017) |
| HLA-B*35:01_YPAPLESLDY | PRA10 | Not known | WO2008118017 A2 |
| (SEQ ID NO: 454) | | | |
| HLA-A*11:01_ATLENLLSH | PRAM4 | Not known | WO2008118017 A2 |
| (SEQ ID NO: 455) | | | |
| HLA-B*51:01_DALLAQKV | PRA12 | Not known | WO2008118017 A2 |
| (SEQ ID NO: 456) | | | |
| HLA-B*44:03_SESDLKHLSW | PRA12 | Not known | WO2008118017 A2 |
| (SEQ ID NO: 457) | | | |
| HLA-A*11:01_ATLENLLSH | PRAM9 | Not known | WO2008118017 A2 |
| (SEQ ID NO: 455) | | | |
| HLA-A*02:07_TLDEYLTYL | PRAM9 | Not known | WO2008118017 A2 |
| (SEQ ID NO: 458) | | | |

One notable example from Table C was KKLC1 HLA-A*01:01_NTDNNLAVY (SEQ ID NO: 5). Kita-kyushu lung cancer antigen-1 (KK-LC-1; CT83) is a cancer testis antigen (CTA) that has been shown to be widely expressed in many different cancer types. It was originally discovered based on a cloned CTL to KK-LC-1 peptide 76-84—RQKRILVNL (SEQ ID NO: 459) (Fukuyama et al., 2006). More recently Stevanović et al., 2017 revealed another peptide from KK-LC-1 recognized by a CTL in a patient with cervical cancer, the predicted peptide KK-LC-1 52-60 NTDNNLAVY (SEQ ID NO: 5). The corresponding TCR for this CTL is now listed on the NIH website https://www.ott.nih.gov/technology/e-153-2016/ and the peptide is listed in WO 2017/089756 A1, herein incorporated by reference, in its entirety, for all purposes.

This example highlights the expected value of predicted HLA-PEPTIDE targets in Table A: Although no information on which CTA HLA-PEPTIDE targets were previously known was incorporated in the prediction, the analysis yielded many targets that were described in the literature, indicating that many of the novel targets can likewise be validated experimentally and ultimately serve as targets for one or more ABPs.

### REFERENCES

Fukuyama, T., Hanagiri, T., Takenoyama, M., Ichiki, Y, Mizukami, M., So, T., Sugaya, M., So, T., Sugio, K., and Yasumoto, K. (2006). Identification of a new cancer/germline gene, KK-LC-1, encoding an antigen recognized by autologous CTL induced on human lung adenocarcinoma. Cancer Res. 66, 4922-4928.
Olsen, L. R., Tongchusak, S., Lin, H., Reinherz, E. L., Brusic, V., and Zhang, G. L. (2017). TANTIGEN: a comprehensive database of tumor T cell antigens. Cancer Immunol. Immunother. CII 66, 731-735.
Stevanović, S., Pasetto, A., Helman, S. R., Gartner, J. J., Prickett, T. D., Howie, B., Robins, H. S., Robbins, P. F., Klebanoff, C. A., Rosenberg, S. A., et al. (2017). Landscape of immunogenic tumor antigens in successful immunotherapy of virally induced epithelial cancer. Science 356, 200-205.
Vita, R., Overton, J. A., Greenbaum, J. A., Ponomarenko, J., Clark, J. D., Cantrell, J. R., Wheeler, D. K., Gabbard, J. L., Hix, D., Sette, A., et al. (2015). The immune epitope database (IEDB) 3.0. Nucleic Acids Res. 43, D405-412.

### Example 42: Identification of Predicted HLA-PEPTIDE Complexes (Table A2)

Next, HLA-peptide targets from proteins of seven genes were identified: AFP, KKLC-1, MAGE-A4, MAGE-A10, MART-1, NY-ESO-1, and WT1.

To identify peptides that are likely to be presented as cell surface antigens by MHC Class I proteins, a sliding window was used to parse each of these proteins into its constituent 8-11 amino acid sequences. These peptides and their flanking sequences were then processed with the HLA peptide presentation deep learning model (see PCT/US2016/067159 and Example 40 above) to calculate the likelihood of presentation of each peptide at an expression level of 100 TPM (high expression) for each of 64 Class I HLA types. Potential modeling artifacts were removed that could give stronger scores to certain HLAs due to training data biases by quantile normalizing model scores for each HLA so that each HLA present scores from the same distribution. In the normalization, the seven target genes as well as 50 randomly selected genes were included to control for HLA allele sequence preferences. A gene was considered likely to be presented if the model normalized score was higher than 0.00075, which was chosen based on the presentation scores of peptides known to be presented in the literature.

The results are shown in Table A2 of International Application No. PCT/US2020/15736 or U.S. application Ser. No. 17/426,627, each of which is hereby incorporated by reference in its entirety

### Example 43: Identification of Antibodies or Antigen-Binding Fragments Thereof that Bind HLA-PEPTIDE Complexes

Overview

The following exemplification demonstrates that antibodies (Abs) can be identified that recognize tumor-specific HLA-restricted peptides. The overall epitope that is recognized by such Abs generally comprises a composite surface of both the peptide as well as the HLA protein presenting that particular peptide. Abs that recognize HLA complexes in a peptide-specific manner are often referred to as T cell receptor (TCR)-like Abs or TCR-mimetic Abs. The HLA-PEPTIDE target antigens that were selected for antibody discovery are HLA-A*01:01_NTDNNLAVY (SEQ ID NO: 5) (Target 33 in Table A1 designated as “G2”) and HLA-A*02:01_LLASSILCA (SEQ ID NO: 8) (Target 6427 in Table A2, designated as “G7”). Cell surface presentation of these HLA-PEPTIDE antigens was confirmed by mass spectrometry analysis of HLA complexes obtained from tumor samples, as described in Example 30.

Generation of HLA-PEPTIDE Target Complexes and Counterscreen Peptide-HLA Complexes, and Stability Analysis

The HLA-PEPTIDE targets G2 and G7, as well as counterscreen negative control peptide-HLAs, were produced recombinantly using conditional ligands for HLA molecules using established methods. In all, 18 counterscreen HLA-peptides were generated for each of the G2 and G7 targets.

Overall Design of Phage Library Screening

The highly diverse SuperHuman 2.0 synthetic naïve scFv library from Distributed Bio Inc (7.6e10 total diversity on ultra-stable and diverse VH/VL scaffolds) was used for phage display. The phage library was initially depleted with 18 pooled negative pHLA complexes (the “complete pool”) followed by three to four rounds of bead-based phage panning with the target pHLA complex using established protocols to identify scFv binders to HLA-PEPTIDE targets G2 and G7, respectively. The phage titer was determined at every round of panning to establish removal of non-binding phage. Phage ELISA results are shown in International Application No. PCT/US2020/1573, which is hereby incorporated by reference in its entirety. There was an enrichment of bound phage in later rounds of panning for each of the G2 and G7 targets. The output phage supernatant was also tested for target binding by ELISA.

The design of target screen 1 for the G2 target is shown in International Application No. PCT/US2020/1573, which is hereby incorporated by reference in its entirety. Similarly, the design of target screen 2 for the G7 target is shown in International Application No. PCT/US2020/1573, which is hereby incorporated by reference in its entirety. Briefly, for each target, three “minipool” counterscreen peptides were selected for their ability to bind the same HLA allele as the target and also to have significantly different ABP-facing features such as charge, bulk, aromatic, or hydrophobic residues. See International Application No. PCT/US2020/1573, which is hereby incorporated by reference in its entirety for G2 and G7. In addition, additional counterscreen peptide-HLA complexes, featuring distinct restricted peptide sequences and different HLA alleles were generated. The 15 additional counterscreen HLA-peptides plus the three “minipool” HLA-peptides formed a “complete pool” of 18 total counterscreen HLA-peptide complexes.

Generation of Peptide-HLA Complexes

α-, and β2 microglobulin chain of various human leukocyte antigens (HLA) were expressed separately in BL21 competent E. coli cells (New England Biolabs) using established procedures (Garboczi, Hung, & Wiley, 1992). Following auto-induction, cells were lysed via sonication in Bugbuster® plus benzonase protein extraction reagent (Novagen). The resulting inclusion bodies were washed and sonicated in wash buffer with and without 0.5% Triton X-100 (50 mM Tris, 100 mM NaCl, 1 mM EDTA). After the final centrifugation, inclusion pellets were dissolved in urea solution (8 M urea, 25 mM MES, 10 mM EDTA, 0.1 mM DTT, pH 6.0). Bradford assay (Biorad) was used to quantify the concentration and the inclusion bodies were stored at −80° C.

HLA complexes were obtained by refolding of recombinantly produced subunits and a synthetically obtained peptide using established procedures. (Garboczi et al., 1992). Briefly, the purified α and β2 microglobulin chains were refolded in refold buffer (100 mM Tris pH 8.0, 400 mM L-Arginine HCl, 2 mM EDTA, 50 mM oxidized glutathione, 5 mM reduced glutathione, protease inhibitor tablet) with the restricted peptide of choice. In some experiments, the restricted peptide of choice was a conditional ligand peptide, which is cleavable upon exposure to a conditional stimulus. In some experiments, the restricted peptide of choice was the G2 or G7 target peptide, or counterscreen peptide. The refold solution was concentrated with a Vivaflow 50 or 50R crossflow cassette (Sartorius Stedim). Three rounds of dialyses in 20 mM Tris pH 8.0 were performed for at least 8 hours each. For the antibody screening and functional assays, the refolded HLA was enzymatically biotinylated using BirA biotin ligase (Avidity). Refolded protein complexes were purified using a HiPrep (16/60 Sephacryl 5200) size exclusion column attached to an Akta FPLC system. Biotinylation was confirmed in a streptavidin gel-shift assay under non-reducing conditions by incubating the refolded protein with an excess of streptavidin at room temperature for 15 minutes prior to SDS-PAGE. The resulting peptide-HLA complexes were aliquoted and stored at −80° C.

Stability Analysis of the Peptide-HLA Complexes

HLA-peptide stability was assessed by conditional ligand peptide exchange and stability ELISA assay. Briefly, conditional ligand-HLA complexes were subjected to ±conditional stimulus in the presence or absence of the counterscreen or test peptides. Exposure to the conditional stimulus cleaves the conditional ligand from the HLA complex, resulting in dissociation of the HLA complex. If the counterscreen or test peptide stably binds the α1/α2 groove of the HLA complex, it “rescues” the HLA complex from disassociation.

The HLA stability ELISA was performed using established procedures. (Chew et al., 2011; Rodenko et al., 2006) A 384-well clear flat bottom polystyrene microplate (Corning) was precoated with 50 μl of streptavidin (Invitrogen) at 2 μg mL⁻¹ in PBS. Following 2 h of incubation at 37° C., the wells were washed with 0.05% Tween 20 in PBS (four times, 50 μL) wash buffer, treated with 50 μl of blocking buffer (2% BSA in PBS), and incubated 30 min at room temperature. Subsequently, 25 μl of peptide-exchanged samples that were 300× diluted with 20 mM Tris HCl/50 mM NaCl were added in quadruplicate. The samples were incubated for 15 min at RT, washed with 0.05% Tween wash buffer (4×50 L), treated for 15 min with 25 μL of HRP-conjugated anti-β2m (1 μg mL⁻¹ in PBS) at RT, washed with 0.05% Tween wash buffer (4×50 μL), and developed for 10-15 min with 25 L of ABTS-solution (Invitrogen), and the reactions were stopped by the addition of 12.5 μL of stop buffer (0.01% sodium azide in 0.1 M citric acid). Absorbance was subsequently measured at 415 nm using a spectrophotometer (SpectraMax i3x; Molecular Devices).

Results for the G2 counterscreen “minipool” and G2 target are shown in International Application No. PCT/US2020/1573, which is hereby incorporated by reference in its entirety. All three counterscreen peptides and the G2 peptide rescued the HLA complex from dissociation.

Results for the additional G2 “complete” pool counterscreen peptides are shown in International Application No. PCT/US2020/1573, which is hereby incorporated by reference in its entirety, demonstrating that they also form stable HLA-peptide complexes.

Results for the G7 counterscreen “minipool” and G7 target are shown in International Application No. PCT/US2020/1573, which is hereby incorporated by reference in its entirety. All three counterscreen peptides and the G7 peptide rescued the HLA complex from dissociation.

Results for the additional G7 “complete” pool counterscreen peptides are shown in International Application No. PCT/US2020/1573, which is hereby incorporated by reference in its entirety, demonstrating that they also form stable HLA-peptide complexes.

Phage Library Screening

Phage library screening was carried out according to the overall screening design described above. Three to four rounds of bead-based panning were performed to identify scFv binders to each peptide-HLA complex. For each round of panning, an aliquot of starting phage was set aside for input titering and the remaining phage was depleted three times against Dynabead M-280 streptavidin beads (Life Technologies) followed by a depletion against Streptavidin beads pre-bound with 100 μmoles of pooled negative peptide-HLA complexes. For the first round of panning, 100 μmoles of peptide-HLA complex bound to streptavidin beads was incubated with depleted phage for 2 hours at room temperature with rotation. Three five-minute washes with 0.5% BSA in 1×PBST (PBS+0.05% Tween-20) followed by three five-minute washes with 0.5% BSA in 1×PBS were utilized to remove any unbound phage to the peptide-HLA complex bound beads. To elute the bound phage from the washed beads, 1 ml 0.1M TEA was added and incubated for 10 minutes at room temperature with rotation. The eluted phage was collected from the beads and neutralized with 0.5 ml 1M Tris-HCl pH 7.5. The neutralized phage was then used to infect log growth TG-1 cells (OD₆₀₀=0.5) and after an hour of infection at 37° C., cells were plated onto 2YT media with 100 μg/ml carbenicillin and 2% glucose (2YTCG) agar plates for output titer and bacterial growth for subsequent panning rounds. For subsequent rounds of panning, selection antigen concentrations were lowered while washes increased by amount and length of wash times at show in Table 25.

TABLE 25
Phage library screening strategy

| Round | Antigen concentration | Washes |
|---|---|---|
| R1 | 100 pmol | 3× PBST + 3× PBS (5 min washes) |
| R2 | 25 pmol | 5 PBST (2× 30 sec, 3× 5 min) + |
| | | 5 PBS (2× 30 sec, 3× 5 min) |
| R3 | 10 pmol | 8 PBST (4× 30 sec, 4× 5 min) + |
| | | 8 PBS (4× 30 sec, 4× 5 min) |
| R4 | 5 pmol, 10 pmol | 30 min PBST + 30 min PBS |

Individual scFvs were cloned from phage and sequenced by DNA Sanger sequencing (“Sequence Unique Binders”). The individual scFvs were also expressed in E. coli and periplasmic extracts (PPE) from E. coli containing the individual crude scFvs were subjected to scFv ELISA

scFv Periplasmic Extract (PPE) ELISA

The individual scFv cloned from phage obtained in the final round of panning, and expressed in E. coli, was subjected to scFv PPE ELISA as follows.

96-well and/or 384-well streptavidin coated plates (Pierce) were coated with 2 μg/ml peptide-HLA complex in HLA buffer and incubated overnight at 4° C. Plates were washed three times between each step with PBST (PBS+0.05%). The antigen coated plates were blocked with 3% BSA in PBS (blocking buffer) for 1 hour at room temperature. After washing, scFv PPEs were added to the plates and incubated at room temperature for 1 hour. Following washing, mouse anti-v5 antibody (Invitrogen) in blocking buffer was added to detect scFv and incubated at room temperature for 1 hour. After washing, HRP-goat anti-mouse antibody (Jackson ImmunoResearch) was added and incubated at room temperature for 1 hour. The plates were then washed three times with PBST and 3 times with PBS before HRP activity was detected with TMB 1-component Microwell Peroxidase Substrate (Seracare) and neutralized with 2N sulfuric acid.

For negative peptide-HLA complex counter-screening, scFv PPE ELISAs were performed as described above, except for the coating antigen. HLA mini-pools consisted of 2 μg/ml of each of the three negative peptide-HLA complexes pooled together and coated onto streptavidin plates for comparison binding to their particular peptide-HLA complex. HLA big pools consisted of 2 μg/ml of each of all 18 negative peptide-HLA complexes pooled together and coated onto streptavidin plates for comparison binding to their particular peptide-HLA complex.

Those scFvs that showed selectivity for target pHLA compared to negative control pHLA by scFv-ELISA as crude PPE, were separately expressed and purified. The purified scFvs were titratated by scFv ELISA for confirmation of binding only target pHLA compared to negative control pHLA (“Selective Binders”).

Clones were formatted into IgG, Fab, or scFv for further biochemical and functional analysis. ScFv clones selected for Fab production to be used for crystallization with their corresponding pHLA complexes were selected based on several parameters: sequence diversity, binding affinity, selectivity, and CDR3 diversity. The clustal software was used to produce a dendrogram and assess the sequence diversity of the Fab clones. The canonical 3D structures of the scFv sequences, based on the VH type, were also considered when possible. Binding affinity, as determined by the equilibrium dissociation constant (K_{D}), was measured using an Octet HTX (ForteBio). Selectivity for the specific peptide-HLA complexes was determined with an ELISA titration of the purified scFvs and compared to negative peptides or streptavidin alone. Cutoff values for the K_{D} and selectivity were determined for each target set based on the range of values obtained for the Fabs within each set. Final clones were then selected to obtain the highest diversity in sequence families and CDR3.

TABLE 26
shows the hit rate for the screening campaign described above.
hit rate for screening campaign

| Group | G2 | G7 |
|---|---|---|
| Gene target | CT83 | CT83 |
| HLA | A*01:01 | A*02:01 |
| Restricted peptide | NTDNNLAVY | LLASSILCA |
| | (SEQ ID NO: 5) | (SEQ ID NO: 8) |
| # Sequence Unique | 74 | 8 |
| Binders | | |
| # Selective Binders | 27 | 6 |
| # selected for IgG | 20 | 8 |
| # selected for Fab | 6 | 3 |
| # selected for scFv | 20 | 7 |

Table 27 shows the VH and VL sequences of the G2 scFv Selective Binders, selective for HLA-PEPTIDE Target HLA-A*01:01_NTDNNLAVY (SEQ ID NO: 5)

Table 28 shows the CDR sequences for the G2 Selective Binders, selective for HLA-PEPTIDE Target HLA-A*01:01_NTDNNLAVY (SEQ ID NO: 5). CDRs were determined according to the Kabat numbering system.

Table 29 shows the VH and VL sequences of the G7 scFv Selective Binders, selective for HLA-PEPTIDE Target HLA-A*02:01_LLASSILCA (SEQ ID NO: 8).

Table 30 shows the CDR sequences for the G7 Selective Binders, selective for HLA-PEPTIDE Target HLA-A*02:01_LLASSILCA (SEQ ID NO: 8). CDRs were determined according to the Kabat numbering system.

### Example 44: In Vivo Proof-of-Concept

Lead antibody or CAR-T constructs are evaluated in vivo to demonstrate directed tumor killing in humanized mouse tumor models. Lead antibody or CAR-T constructs are evaluated in xenograft tumor models engrafted with human tumors and PBMCs. Anti-tumor activity is measured and compared to control constructs to demonstrate target-specific tumor killing.

While the invention has been particularly shown and described with reference to certain embodiments and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### Sequences

TABLE 4
VH and VL sequences of scFv hits that bind target G5

| Target | Clone | | |
|---|---|---|---|
| group | name | V_{H} | V_{L} |
| G5 | G5(7E07) | QVQLVQSGAEVKKPGASVKVSCK | DIVMTQSPLSLPVTPGEPASISCRSS |
| | | ASGYTFTSYDINWVRQAPGQGLE | QSLLHSNGYNYLDWYLQKPGQSP |
| | | WMGIINPRSGSTKYAQKFQGRVT | QLLIYLGSYRASGVPDRFSGSGSGT |
| | | MTRDTSTSTVYMELSSLRSEDTAV | DFTLKISRVEAEDVGVYYCMQGL |
| | | YYCARDGVRYYGMDVWGQGTTV | QTPITFGQGTRLEIK (SEQ ID NO: |
| | | TVSS (SEQ ID NO: 141) | 159) |
| G5 | G5(7B03) | QVQLVQSGAEVKKPGSSVKVSCK | DIVMTQSPLSLPVTPGEPASISCRSS |
| | | ASGYTFTSHDINWVRQAPGQGLE | QSLLHSNGYNYLDWYLQKPGQSP |
| | | WMGWMNPNSGDTGYAQKFQGR | QLLIYLGSSRASGVPDRFSGSGSGT |
| | | VTITADESTSTAYMELSSLRSEDTA | DFTLKISRVEAEDVGVYYCMQAL |
| | | VYYCARGVRGYDRSAGYWGQGT | QTPPTFGPGTKVDIK (SEQ ID NO: |
| | | LVIVSS (SEQ ID NO: 142) | 160) |
| G5 | G5(7A05) | EVQLLESGGGLVKPGGSLRLSCAA | DIQMTQSPSSLSASVGDRVTITCQA |
| | | SGFSFSSYWMSWVRQAPGKGLEW | SQDISNYLNWYQQKPGKAPKLLIY |
| | | ISYISGDSGYTNYADSVKGRFTISR | AASSLQSGVPSRFSGSGSGTDFTLT |
| | | DDSKNTLYLQMNSLKTEDTAVYY | ISSLQPEDFATYYCQQAISFPLTFG |
| | | CASHDYGDYGEYFQHWGQGTLV | QSTKVEIK (SEQ ID NO: 161) |
| | | TVSS (SEQ ID NO: 143) | |
| G5 | G5(7F06) | EVQLLQSGGGLVQPGGSLRLSCAA | DIQMTQSPSSLSASVGDRVTITCRA |
| | | SGFTFSNSDMNWVRQAPGKGLEW | SQSISSWLAWYQQKPGKAPKLLIY |
| | | VAYISSGSSTIYYADSVKGRFTISR | SASTLQSGVPSRFSGSGSGTDFTLT |
| | | DNSKNTLYLQMNSLRAEDTAVYY | ISSLQPEDFATYYCQQANSFPLTFG |
| | | CARVSWYCSSTSCGVNWFDPWGQ | GGTKVEIK (SEQ ID NO: 162) |
| | | GTLVTVSS (SEQ ID NO: 144) | |
| G5 | G5(1B12) | EVQLLESGGGLVQPGGSLRLSCAA | DIQMTQSPSSLSASVGDRVTITCRA |
| | | SGFTFSNSDMNWVRQAPGKGLEW | SQSISSWLAWYQQKPGKAPKLLIY |
| | | VASISSSGGYINYADSVKGRFTISR | AASSLQSGVPSRFSGSGSGTDFTLT |
| | | DNSKNTLYLQMNSLRAEDTAVYY | ISSLQPEDFATYYCQQANSFPLTFG |
| | | CAKVNWNDGPYFDYWGQGTLVT | GGTKVEIK (SEQ ID NO: 163) |
| | | VSS (SEQ ID NO: 145) | |
| G5 | G5(1C12) | QVQLVQSGAEVKKPGSSVKVSCK | DIQMTQSPSSLSASVGDRVTITCRA |
| | | ASGGTFSNFGVSWLRQAPGQGLE | SQSISSWLAWYQQKPGKAPKLLIY |
| | | WMGGIIPILGTANYAQKFQGRVTI | AASTLQSGVPSRFSGSGSGTDFTLT |
| | | TADESTSTAYMELSSLRSEDTAVY | ISSLQPEDFATYYCQQSYSIPLTFG |
| | | YCATPTNSGYYGPYYYYGMDVW | GGTKVEIK (SEQ ID NO: 164) |
| | | GQGTTVTVSS (SEQ ID NO: 146) | |
| G5 | G5(1E05) | QVQLVQSGAEVKKPGASVKVSCK | DIQMTQSPSSLSASVGDRVTITCRA |
| | | ASGYTFTSYNMHWVRQAPGQGLE | SQGISNYLNWYQQKPGKAPKLLIY |
| | | WMGWINPNSGGTNYAQKFQGRV | YASSLQSGVPSRFSGSGSGTDFTLT |
| | | TMTRDTSTSTVYMELSSLRSEDTA | ISSLQPEDFATYYCQQTYMMPYTF |
| | | VYYCARDVMDVWGQGTTVTVSS | GQGTKVEIK (SEQ ID NO: 165) |
| | | (SEQ ID NO: 147) | |
| G5 | G5(3G01) | QVQLVQSGAEVKKPGASVKVSCK | DIQMTQSPSSLSASVGDRVTITCRA |
| | | ASGGTFSGYLVSWVRQAPGQGLE | SQSISSYLNWYQQKPGKAPKLLIY |
| | | WMGWINPNSGGTNTAQKFQGRVT | GASSLQSGVPSRFSGSGSGTDFTLT |
| | | MTRDTSTSTVYMELSSLRSEDTAV | ISSLQPEDFATYYCQQSYITPWTFG |
| | | YYCAREGYGMDVWGQGTTVTVS | QGTKVEIK (SEQ ID NO: 166) |
| | | S (SEQ ID NO: 148) | |
| G5 | G5(3G08) | QVQLVQSGAEVKKPGASVKVSCK | DIQMTQSPSSLSASVGDRVTITCRA |
| | | ASGYIFRNYPMHWVRQAPGQGLE | SQGISNYLAWYQQKPGKAPKLLIY |
| | | WMGWINPDSGGTKYAQKFQGRV | AASSLQSGVPSRFSGSGSGTDFTLT |
| | | TMTRDTSTSTVYMELSSLRSEDTA | ISSLQPEDFATYYCQQSYITPYTFG |
| | | VYYCARDNGVGVDYWGQGTLVT | QGTKLEIK (SEQ ID NO: 167) |
| | | VSS (SEQ ID NO: 149) | |
| G5 | G5(4B02) | QVQLVQSGAEVKKPGASVKVSCK | DIVMTQSPDSLAVSLGERATINCK |
| | | ASGYTFTGYYMHWVRQAPGQGL | TSQSVLYRPNNENYLAWYQQKPG |
| | | EWMGWMNPNIGNTGYAQKFQGR | QPPKLLIYQASIREPGVPDRFSGSG |
| | | VTMTRDTSTSTVYMELSSLRSEDT | SGTDFTLTISSLQAEDVAVYYCQQ |
| | | AVYYCARGIADSGSYYGNGRDYY | YYTTPYTFGQGTKLEIK (SEQ ID |
| | | YGMDVWGQGTTVTVSS (SEQ ID | NO: 168) |
| | | NO: 150) | |
| G5 | G5(4E04) | QVQLVQSGAEVKKPGASVKVSCK | DIQMTQSPSSLSASVGDRVTITCRA |
| | | ASGGTFSSYGISWVRQAPGQGLE | SQSISRFLNWYQQKPGKAPKLLIY |
| | | WMGWINPNSGVTKYAQKFQGRV | GASRPQSGVPSRFSGSGSGTDFTLT |
| | | TMTRDTSTSTVYMELSSLRSEDTA | ISSLQPEDFATYYCQQSYSTPLTFG |
| | | VYYCARGDYYFDYWGQGTLVTV | QGTKVEIK (SEQ ID NO: 169) |
| | | SS (SEQ ID NO: 151) | |
| G5 | G5(1D06) | QVQLVQSGAEVKKPGASVKVSCK | DIVMTQSPLSLPVTPGEPASISCRSS |
| | | ASGYTFTSYDINWVRQAPGQGLE | QSLLHSNGYNYLDWYLQKPGQSP |
| | | WMGWINPNSGDTKYSQKFQGRVT | QLLIYLGSHRASGVPDRFSGSGSGT |
| | | MTRDTSTSTVYMELSSLRSEDTAV | DFTLKISRVEAEDVGVYYCMQAL |
| | | YYCARDGTRYYGMDVWGQGTTV | QTPLTFGGGTKVEIK (SEQ ID NO: |
| | | TVSS (SEQ ID NO: 152) | 170) |
| G5 | G5(1H11) | EVQLLESGGGLVKPGGSLRLSCAA | EIVMTQSPATLSVSPGERATLSCRA |
| | | SGFTFSDYYMSWVRQAPGKGLEW | SQSVSSNLAWYQQKPGQAPRLLIY |
| | | VSYISSSSSYTNYADSVKGRFTISR | AASARASGIPARFSGSGSGTEFTLT |
| | | DDSKNTLYLQMNSLKTEDTAVYY | ISSLQSEDFAVYYCQQYGSWPRTF |
| | | CARDVVANFDYWGQGTLVTVSS | GQGTKVEIK (SEQ ID NO: 171) |
| | | (SEQ ID NO: 153) | |
| G5 | G5(2B10) | QVQLVQSGAEVKKPGASVKVSCK | DIQMTQSPSSLSASVGDRVTITCRA |
| | | ASGGTFSSYAISWVRQAPGQGLE | SQSISSYLNWYQQKPGKAPKLLIY |
| | | WMGWMNPDSGSTGYAQRFQGRV | GASRLQSGVPSRFSGSGSGTDFTLT |
| | | TMTRDTSTSTVYMELSSLRSEDTA | ISSLQPEDFATYYCQQSYSTPVTFG |
| | | VYYCARGHSSGWYYYYGMDVW | QGTKVEIK (SEQ ID NO: 172) |
| | | GQGTTVTVSS (SEQ ID NO: 154) | |
| G5 | G5(2H08) | EVQLLESGGGLVQPGGSLRLSCAA | DIVMTQSPLSLPVTPGEPASISCRSS |
| | | SGFTFTSYSMHWVRQAPGKGLEW | QSLLHSNGYNYLDWYLQKPGQSP |
| | | VSSITSFTNTMYYADSVKGRFTISR | QLLIYLGSNRASGVPDRFSGSGSGT |
| | | DNSKNTLYLQMNSLRAEDTAVYY | DFTLKISRVEAEDVGVYYCMQAL |
| | | CAKDLGSYGGYYWGQGTLVTVSS | QTPYTFGQGTKVEIK (SEQ ID NO: |
| | | (SEQ ID NO: 155) | 173) |
| G5 | G5(3G05) | QVQLVQSGAEVKKPGASVKVSCK | DIQMTQSPSSLSASVGDRVTITCQA |
| | | ASGYTFTNYYMHWVRQAPGQGL | SEDISNHLNWYQQKPGKAPKLLIY |
| | | EWMGIINPSGGSTSYAQKFQGRVT | DALSLQSGVPSRFSGSGSGTDFTLT |
| | | MTRDTSTSTVYMELSSLRSEDTAV | ISSLQPEDFATYYCQQANSFPFTFG |
| | | YYCARSWFGGFNYHYYGMDVWG | PGTKVDIK (SEQ ID NO: 174) |
| | | QGTTVTVSS (SEQ ID NO: 156) | |
| G5 | G5(4A07) | QVQLVQSGAEVKKPGASVKVSCK | DIVMTQSPLSLPVTPGEPASISCRSS |
| | | ASGYTFTSYYMHWVRQAPGQGLE | QSLLHSNGYNYLDWYLQKPGQSP |
| | | WMGWMNPNSGNTGYAQKFQGR | QLLIYLGSNRASGVPDRFSGSGSGT |
| | | VTMTRDTSTSTVYMELSSLRSEDT | DFTLKISRVEAEDVGVYYCMQAL |
| | | AVYYCARELPIGYGMDVWGQGTT | QTPLTFGQGTKVEIK (SEQ ID NO: |
| | | VTVSS (SEQ ID NO: 157) | 175) |
| G5 | G5(4B01) | QVQLVQSGAEVKKPGSSVKVSCK | DIQMTQSPSSLSASVGDRVTITCRA |
| | | ASGGTFSSYAISWVRQAPGQGLE | SQSISSYLNWYQQKPGKAPKLLIY |
| | | WMGGIIPIVGTANYAQKFQGRVTI | AASSLQSGVPSRFSGSGSGTDFTLT |
| | | TADESTSTAYMELSSLRSEDTAVY | ISSLQPEDFATYYCQQSYSTPLTFG |
| | | YCARGGSYYYYGMDVWGQGTTV | GGTKVEIK (SEQ ID NO: 176) |
| | | TVSS (SEQ ID NO: 158) | |

TABLE 5
CDR sequences of identified scFvs to G5, numbered according to the
Kabat numbering scheme

| Target | Clone | | | | | | |
|---|---|---|---|---|---|---|---|
| group | name | HCDR1 | HCDR2 | HCDR3 | LCDR1 | LCDR2 | LCDR3 |
| G5 | G5(7E07) | YTFTS | GIINPRS | CARDGVR | RSSQSLLH | LGSYR | CMQGLQ |
| | | YDIN | GSTKYA | YYGMDV | SNGYNYL | AS (SEQ | TPITF |
| | | (SEQ ID | (SEQ ID | W (SEQ ID | D (SEQ ID | ID NO: | (SEQ ID |
| | | NO: | NO: 437) | NO: 67) | NO: 438) | 439) | NO: 85) |
| | | 436) | | | | | |
| G5 | G5(7B03) | YTFTS | GWMNP | CARGVRG | RSSQSLLH | LGSSR | CMQALQ |
| | | HDIN | NSGDTG | YDRSAGY | SNGYNYL | AS (SEQ | TPPTF |
| | | (SEQ ID | YA (SEQ | W (SEQ ID | D (SEQ ID | ID NO: | (SEQ ID |
| | | NO: | ID NO: | NO: 68) | NO: 438) | 462) | NO: 86) |
| | | 460) | 461) | | | | |
| G5 | G5(7A05) | FSFSSY | SYISGDS | CASHDYG | QASQDISN | AASSL | CQQAISF |
| | | WMS | GYTNYA | DYGEYFQ | YLN (SEQ | QS (SEQ | PLTF |
| | | (SEQ ID | (SEQ ID | HW (SEQ | ID NO: 465) | ID NO: | (SEQ ID |
| | | NO: | NO: 464) | ID NO: 69) | | 466) | NO: 87) |
| | | 463) | | | | | |
| G5 | G5(7F06) | FTFSNS | AYISSGS | CARVSWY | RASQSISS | SASTLQ | CQQANS |
| | | DMN | STIYYA | CSSTSCGV | WLA (SEQ | S (SEQ | FPLTF |
| | | (SEQ ID | (SEQ ID | NWFDPW | | | |
| | | NO: | NO: 468) | (SEQ ID | ID NO: 469) | ID NO: | (SEQ ID |
| | | 467) | | NO: 70) | | 470) | NO: 88) |
| G5 | G5(1B12) | FTFSNS | ASISSSG | CAKVNW | RASQSISS | AASSL | CQQANS |
| | | DMN | GYINYA | NDGPYFD | WLA (SEQ | QS (SEQ | FPLTF |
| | | (SEQ ID | (SEQ ID | YW (SEQ | | ID NO: | (SEQ ID |
| | | NO: | NO: 471) | ID NO: 71) | ID NO: 469) | 466) | NO: 88) |
| | | 467) | | | | | |
| G5 | G5(1C12) | GTFSNF | GGIIPILG | CATPTNS | RASQSISS | AASTL | CQQSYSI |
| | | GVS | TANYA | GYYGPYY | WLA (SEQ | QS (SEQ | PLTF |
| | | (SEQ ID | (SEQ ID | YYGMDV | ID NO: 469) | ID NO: | (SEQ ID |
| | | NO: | NO: 473) | W (SEQ ID | | 474) | NO: 59) |
| | | 472) | | NO: 72) | | | |
| G5 | G5(1E05) | YTFTS | GWINPN | CARDVM | RASQGISN | YASSL | CQQTYM |
| | | YNMH | SGGTNY | DVW (SEQ | YLN (SEQ | QS (SEQ | MPYTF |
| | | (SEQ ID | A (SEQ ID | ID NO: 73) | ID NO: 477) | ID NO: | (SEQ ID |
| | | NO: | NO: 476) | | | 478) | NO: 89) |
| | | 475) | | | | | |
| G5 | G5(3G01) | GTFSG | GWINPN | CAREGYG | RASQSISS | GASSL | CQQSYIT |
| | | YLVS | SGGTNT | MDVW | YLN (SEQ | QS (SEQ | PWTF |
| | | (SEQ ID | A (SEQ ID | (SEQ ID | ID NO: 481) | ID NO: | (SEQ ID |
| | | NO: | NO: 480) | NO: 74) | | 482) | NO: 90) |
| | | 479) | | | | | |
| G5 | G5(3G08) | YIFRNY | GWINPD | CARDNGV | RASQGISN | AASSL | CQQSYIT |
| | | PMH | SGGTKY | GVDYW | YLA (SEQ | QS (SEQ | PYTF |
| | | (SEQ ID | A (SEQ ID | (SEQ ID | ID NO: 485) | ID NO: | (SEQ ID |
| | | NO: | NO: 484) | NO: 75) | | 466) | NO: 91) |
| | | 483) | | | | | |
| G5 | G5(4B02) | YTFTG | GWMNP | CARGIAD | KTSQSVL | QASIRE | CQQYYT |
| | | YYMH | NIGNTG | SGSYYGN | YRPNNEN | P (SEQ | TPYTF |
| | | (SEQ ID | YA (SEQ | GRDYYYG | YLA (SEQ | ID NO: | (SEQ ID |
| | | NO: | ID NO: | MDVW | ID NO: 488) | 489) | NO: 92) |
| | | 486) | 487) | (SEQ ID | | | |
| | | | | NO: 76) | | | |
| G5 | G5(4E04) | GTFSSY | GWINPN | CARGDYY | RASQSISR | GASRP | CQQSYS |
| | | GIS | SGVTKY | FDYW | FLN (SEQ | QS (SEQ | TPLTF |
| | | (SEQ ID | A (SEQ ID | (SEQ ID | ID NO: 492) | ID NO: | (SEQ ID |
| | | NO: | NO: 491) | NO: 77) | | 493) | NO: 55) |
| | | 490) | | | | | |
| G5 | G5(1D06) | YTFTS | GWINPN | CARDGTR | RSSQSLLH | LGSHR | CMQALQ |
| | | YDIN | SGDTKY | YYGMDV | SNGYNYL | AS (SEQ | TPLTF |
| | | (SEQ ID | S (SEQ ID | W (SEQ ID | D (SEQ ID | ID NO: | (SEQ ID |
| | | NO: | NO: 494) | NO: 78) | NO: 438) | 495) | NO: 93) |
| | | 436) | | | | | |
| G5 | G5(1H11) | FTFSDY | SYISSSSS | CARDVVA | RASQSVSS | AASAR | CQQYGS |
| | | YMS | YTNYA | NFDYW | NLA (SEQ | AS (SEQ | WPRTF |
| | | (SEQ ID | (SEQ ID | (SEQ ID | ID NO: 498) | ID NO: | (SEQ ID |
| | | NO: | NO: 497) | NO: 79) | | 499) | NO: 94) |
| | | 496) | | | | | |
| G5 | G5(2B10) | GTFSSY | GWMNP | CARGHSS | RASQSISS | GASRL | CQQSYS |
| | | AIS | DSGSTG | GWYYYY | YLN (SEQ | QS (SEQ | TPVTF |
| | | (SEQ ID | YA (SEQ | GMDVW | ID NO: 481) | ID NO: | (SEQ ID |
| | | NO: | ID NO: | (SEQ ID | | 502) | NO: 95) |
| | | 500) | 501) | NO: 80) | | | |
| G5 | G5(2H08) | FTFTSY | SSITSFTN | CAKDLGS | RSSQSLLH | LGSNR | CMQALQ |
| | | SMH | TMYYA | YGGYYW | SNGYNYL | AS (SEQ | TPYTF |
| | | (SEQ ID | (SEQ ID | (SEQ ID | D (SEQ ID | ID NO: | (SEQ ID |
| | | NO: | NO: 504) | NO: 81) | NO: 438) | 505) | NO: 96) |
| | | 503) | | | | | |
| G5 | G5(3G05) | YTFTN | GIINPSG | CARSWFG | QASEDISN | DALSL | CQQANS |
| | | YYMH | GSTSYA | GFNYHYY | HLN (SEQ | QS (SEQ | FPFTF |
| | | (SEQ ID | (SEQ ID | GMDVW | ID NO: 508) | ID NO: | (SEQ ID |
| | | NO: | NO: 507) | (SEQ ID | | 509) | NO: 97) |
| | | 506) | | NO: 82) | | | |
| G5 | G5(4A07) | YTFTS | GWMNP | CARELPIG | RSSQSLLH | LGSNR | CMQALQ |
| | | YYMH | NSGNTG | YGMDVW | SNGYNYL | AS (SEQ | TPLTF |
| | | (SEQ ID | YA (SEQ | (SEQ ID | D (SEQ ID | ID NO: | (SEQ ID |
| | | NO: | ID NO: | NO: 83) | NO: 438) | 505) | NO: 93) |
| | | 510) | 511) | | | | |
| G5 | G5(4B01) | GTFSSY | GGIIPVM | CARGGSY | RASQSISS | AASSL | CQQSYS |
| | | AIS | GTGNYA | YYYGMD | YLN (SEQ | QS (SEQ | TPLTF |
| | | (SEQ ID | (SEQ ID | VW (SEQ | ID NO: 481) | ID NO: | (SEQ ID |
| | | NO: | NO: 512) | ID NO: 84) | | 466) | NO: 55) |
| | | 500) | | | | | |

VH and sequences of scFv hits that bind

| Target | Clone | | |
|---|---|---|---|
| group | name | V_{H} | V_{L} |
| G8 | G8(1A03) | QVQLVQSGAEVKKPGASVKVSCK | DIQMTQSPSSLSASVGDRVTITCRA |
| | | ASGGTFSRSAITWVRQAPGQGLE | SQSITSYLNWYQQKPGKAPKLLIY |
| | | WMGWINPNSGATNYAQKFQGRV | DASNLETGVPSRFSGSGSGTDFTLT |
| | | TMTRDTSTSTVYMELSSLRSEDTA | ISSLQPEDFATYYCQQNYNSVTFG |
| | | VYYCARDDYGDYVAYFQHWGQG | QGTKLEIK (SEQ ID NO: 223) |
| | | TLVTVSS (SEQ ID NO: 206) | |
| G8 | G8(1A04) | QVQLVQSGAEVKKPGASVKVSCK | DIQMTQSPSSLSASVGDRVTITCW |
| | | ASGYPFIGQYLHWVRQAPGQGLE | ASQGISSYLAWYQQKPGKAPKLLI |
| | | WMGIINPSGDSATYAQKFQGRVT | YAASSLQSGVPSRFSGSGSGTDFTL |
| | | MTRDTSTSTVYMELSSLRSEDTAV | TISSLQPEDFATYYCQQSYNTPWT |
| | | YYCARDLSYYYGMDVWGQGTTV | FGPGTKVDIK (SEQ ID NO: 224) |
| | | TVSS (SEQ ID NO: 207) | |
| G8 | G8(1A06) | QVQLVQSGAEVKKPGASVKVSCK | DIQMTQSPSSLSASVGDRVTITCRA |
| | | ASGYTFTNYYMHWVRQAPGQGL | SQAISNSLAWYQQKPGKAPKLLIY |
| | | EWMGWMNPIGGGTGYAQKFQGR | AASTLQSGVPSRFSGSGSGTDFTLT |
| | | VTMTRDTSTSTVYMELSSLRSEDT | ISSLQPEDFATYYCGQSYSTPPTFG |
| | | AVYYCARVYDFWSVLSGFDIWGQ | QGTKLEIK (SEQ ID NO: 225) |
| | | GTLVTVSS (SEQ ID NO: 208) | |
| G8 | G8(1B03) | EVQLLESGGGLVQPGGSLRLSCAA | DIQMTQSPSSLSASVGDRVTITCRA |
| | | SGFTFSDYYMSWVRQAPGKGLEW | SQSISSYLNWYQQKPGKAPKLLIY |
| | | VSGINWNGGSTGYADSVKGRFTIS | KASSLESGVPSRFSGSGSGTDFTLT |
| | | RDNSKNTLYLQMNSLRAEDTAVY | ISSLQPEDFATYYCQQSYSAPYTFG |
| | | YCARVEQGYDIYYYYYMDVWGK | PGTKVDIK (SEQ ID NO: 226) |
| | | GTTVTVSS (SEQ ID NO: 209) | |
| G8 | G8(1C11) | QVQLVQSGAEVKKPGASVKVSCK | DIQMTQSPSSLSASVGDRVTITCQA |
| | | ASGGTLSSYPINWVRQAPGQGLE | SQDISNYLNWYQQKPGKAPKLLIY |
| | | WMGWISTYSGHADYAQKLQGRV | AASSLQSGVPSRFSGSGSGTDFTLT |
| | | TMTRDTSTSTVYMELSSLRSEDTA | ISSLQPEDFATYYCQQSYSIPPTFG |
| | | VYYCARSYDYGDYLNFDYWGQG | GGTKVDIK (SEQ ID NO: 227) |
| | | TLVTVSS (SEQ ID NO: 210) | |
| G8 | G8(1D02) | EVQLLESGGGLVQPGGSLRLSCAA | DIQMTQSPSSLSASVGDRVTITCQA |
| | | SGFTFSSYWMSWVRQAPGKGLEW | SQDISNYLNWYQQKPGKAPKLLIY |
| | | VSSISGRGDNTYYADSVKGRFTISR | AASSLQSGVPSRFSGSGSGTDFTLT |
| | | DNSKNTLYLQMNSLRAEDTAVYY | ISSLQPEDFATYYCQQSYSAPYTFG |
| | | CARASGSGYYYYYGMDVWGQGT | GGTKVEIK (SEQ ID NO: 228) |
| | | TVTVSS (SEQ ID NO: 211) | |
| G8 | G8(1H08) | QVQLVQSGAEVKKPGASVKVSCK | DIQMTQSPSSLSASVGDRVTITCRA |
| | | ASGYTFGNYFMHWVRQAPGQGLE | SQGINSYLAWYQQKPGKAPKLLIY |
| | | WMGMVNPSGGSETFAQKFQGRVT | DASNLETGVPSRFSGSGSGTDFTLT |
| | | MTRDTSTSTVYMELSSLRSEDTAV | ISSLQPEDFATYYCQQHNSYPPTFG |
| | | YYCAASTWIQPFDYWGQGTLVTV | QGTKLEIK (SEQ ID NO: 229) |
| | | SS (SEQ ID NO: 212) | |
| G8 | G8(2B05) | EVQLLESGGGLVQPGGSLRLSCAA | DIQMTQSPSSLSASVGDRVTITCRA |
| | | SGFDFSIYSMNWVRQAPGKGLEW | SQSISRWLAWYQQKPGKAPKLLIY |
| | | VSAISGSGGSTYYADSVKGRFTISR | AASSLQSGVPSRFSGSGSGTDFTLT |
| | | DNSKNTLYLQMNSLRAEDTAVYY | ISSLQPEDFATYYCQQYSTYPITIG |
| | | CASNGNYYGSGSYYNYWGQGTL | QGTKVEIK (SEQ ID NO: 230) |
| | | VTVSS (SEQ ID NO: 213) | |
| G8 | G8(2E06) | QVQLVQSGAEVKKPGASVKVSCK | DIQMTQSPSSLSASVGDRVTITCRA |
| | | ASGYTLTTYYMHWVRQAPGQGLE | SQGISNSLAWYQQKPGKAPKLLIY |
| | | WMGWINPNSGGTNYAQKFQGRV | AASSLQSGVPSRFSGSGSGTDFTLT |
| | | TMTRDTSTSTVYMELSSLRSEDTA | ISSLQPEDFATYYCQQANSFPWTF |
| | | VYYCARAVYYDFWSGPFDYWGQ | GQGTKLEIK (SEQ ID NO: 231) |
| | | GTLVTVSS (SEQ ID NO: 214) | |
| G8 | G8(2C10) | QVQLVQSGAEVKKPGASVKVSCK | DIQMTQSPSSLSASVGDRVTITCRA |
| | | ASGYTFTSYYMHWVRQAPGQGLE | SQDVSTWLAWYQQKPGKAPKLLI |
| | | WMGWINPYSGGTNYAQKFQGRV | YAASSLQSGVPSRFSGSGSGTDFTL |
| | | TMTRDTSTSTVYMELSSLRSEDTA | TISSLQPEDFATYYCQQSHSTPQTF |
| | | VYYCAKGGIYYGSGSYPSWGQGT | GQGTKVEIK (SEQ ID NO: 232) |
| | | LVTVSS (SEQ ID NO: 215) | |
| G8 | G8(2E04) | QVQLVQSGAEVKKPGSSVKVSCK | DIQMTQSPSSLSASVGDRVTITCRA |
| | | ASGGTFSSYGVSWVRQAPGQGLE | SQSISSWLAWYQQKPGKAPKLLIY |
| | | WMGWISPYSGNTDYAQKFQGRVT | DASNLETGVPSRFSGSGSGTDFTLT |
| | | ITADESTSTAYMELSSLRSEDTAVY | ISSLQPEDFATYYCQQSYSTPLTFG |
| | | YCARGLYYMDVWGKGTTVTVSS | GGTKLEIK (SEQ ID NO: 233) |
| | | (SEQ ID NO: 216) | |
| G8 | G8(4F05) | QVQLVQSGAEVKKPGASVKVSCK | DIQMTQSPSSLSASVGDRVTITCRA |
| | | ASGYTFSNMYLHWVRQAPGQGLE | SQGISNYLAWYQQKPGKAPKLLIY |
| | | WMGWINPNTGDTNYAQTFQGRV | AASTLQSGVPSRFSGSGSGTDFTLT |
| | | TMTRDTSTSTVYMELSSLRSEDTA | ISSLQPEDFATYYCQQSYSTPLTFG |
| | | VYYCARGLYGDYFLYYGMDVWG | GGTKVEIK (SEQ ID NO: 234) |
| | | QGTKVTVSS (SEQ ID NO: 217) | |
| G8 | G8(5C03) | QVQLVQSGAEVKKPGASVKVSCK | DIQMTQSPSSLSASVGDRVTITCRA |
| | | ASGYTFTSYYMHWVRQAPGQGLE | SQGISNWLAWYQQKPGKAPKLLI |
| | | WMGWMNPNSGNTGYAQKFQGR | YAASTLQSGVPSRFSGSGSGTDFTL |
| | | VTMTRDTSTSTVYMELSSLRSEDT | TISSLQPEDFATYYCQQTYSTPWTF |
| | | AVYYCARGLLGFGEFLTYGMDV | GQGTKLEIK (SEQ ID NO: 235) |
| | | WGQGTLVTVSS (SEQ ID NO: 218) | |
| G8 | G8(5F02) | QVQLVQSGAEVKKPGASVKVSCK | EIVMTQSPATLSVSPGERATLSCRA |
| | | ASGYTFTGYYIHWVRQAPGQGLE | SQSVGNSLAWYQQKPGQAPRLLIY |
| | | WMGVINPSGGSTTYAQKLQGRVT | GASTRATGIPARFSGSGSGTEFTLTI |
| | | MTRDTSTSTVYMELSSLRSEDTAV | SSLQSEDFAVYYCQQYGSSPYTFG |
| | | YYCARDRDSSWTYYYYGMDVWG | QGTKVEIK (SEQ ID NO: 236) |
| | | QGTTVTVSS (SEQ ID NO: 219) | |
| G8 | G8(5G08) | QVQLVQSGAEVKKPGASVKVSCK | DIQMTQSPSSLSASVGDRVTITCRA |
| | | ASGYTFTSNYMHWVRQAPGQGLE | SQSISGYLNWYQQKPGKAPKLLIY |
| | | WMGWMNPNSGNTGYAQKFQGR | AASSLQSGVPSRFSGSGSGTDFTLT |
| | | VTMTRDTSTSTVYMELSSLRSEDT | ISSLQPEDFATYYCQQSHSTPLTFG |
| | | AVYYCARGLYGDYFLYYGMDVW | QGTKVEIK (SEQ ID NO: 237) |
| | | GQGTTVTVSS (SEQ ID NO: 220) | |
| G8 | G8(1C01) | QVQLVQSGAEVKKPGASVKVSCK | DIQMTQSPSSLSASVGDRVTITCRA |
| | | ASGGTFSSHAISWVRQAPGQGLE | SQNIYTYLNWYQQKPGKAPKLLIY |
| | | WMGVIIPSGGTSYTQKFQGRVTMT | DASNLETGVPSRFSGSGSGTDFTLT |
| | | RDTSTSTVYMELSSLRSEDTAVYY | ISSLQPEDFATYYCQQANGFPLTFG |
| | | CARGDYYDSSGYYFPVYFDYWGQ | GGTKVEIK (SEQ ID NO: 238) |
| | | GTLVTVSS (SEQ ID NO: 221) | |
| G8 | G8(2C11) | QVQLVQSGAEVKKPGASVKVSCK | DIQMTQSPSSLSASVGDRVTITCRA |
| | | ASGYTFTSYAMNWVRQAPGQGLE | SQSISSYLNWYQQKPGKAPKLLIY |
| | | WMGWINPNSGGTNYAQKFQGRV | AASSLQSGVPSRFSGSGSGTDFTLT |
| | | TMTRDTSTSTVYMELSSLRSEDTA | ISSLQPEDFATYYCQQSYSTPLTFG |
| | | VYYCARDPFWSGHYYYYGMDVW | GGTKVEIK (SEQ ID NO: 176) |
| | | GQGTTVTVSS (SEQ ID NO: 222) | |

TABLE 7
CDR sequences of identified scFvs to G8, numbered according to the Kabat
numbering scheme

| Target | Clone | | | | | | |
|---|---|---|---|---|---|---|---|
| group | name | HCDR1 | HCDR2 | HCDR3 | LCDR1 | LCDR2 | LCDR3 |
| G8 | G8(1A03) | GTFSRS | GWINPN | CARDDYG | RASQSITS | DASNL | CQQNYN |
| | | AIT | SGATNY | DYVAYFQ | YLN (SEQ | ET (SEQ | SVTF |
| | | (SEQ ID | A (SEQ ID | HW (SEQ | ID NO: 515) | ID NO: | (SEQ ID |
| | | NO: | NO: 514) | ID NO: | | 516) | NO: 194) |
| | | 513) | | 177) | | | |
| G8 | G8(1A04) | YPFIGQ | GIINPSG | CARDLSY | WASQGISS | AASSL | CQQSYN |
| | | YLH | DSATYA | YYGMDV | YLA (SEQ | QS (SEQ | TPWTF |
| | | (SEQ ID | (SEQ ID | W (SEQ ID | ID NO: 519) | ID NO: | (SEQ ID |
| | | NO: | NO: 518) | NO: 178) | | 466) | NO: 195) |
| | | 517) | | | | | |
| G8 | G8(1A06) | YTFTN | GWMNPI | CARVYDF | RASQAISN | AASTL | CGQSYS |
| | | YYMH | GGGTGY | WSVLSGF | SLA (SEQ | QS (SEQ | TPPTF |
| | | (SEQ ID | A (SEQ ID | DIW (SEQ | ID NO: 521) | ID NO: | (SEQ ID |
| | | NO: | NO: 520) | ID NO: | | 474) | NO: 196) |
| | | 506) | | 179) | | | |
| G8 | G8(1B03) | FTFSDY | SGINWN | CARVEQG | RASQSISS | KASSLE | CQQSYS |
| | | YMS | GGSTGY | YDIYYYY | YLN (SEQ | S (SEQ | APYTF |
| | | (SEQ ID | A (SEQ ID | YMDVW | ID NO: 481) | ID NO: | (SEQ ID |
| | | NO: | NO: 522) | (SEQ ID | | 523) | NO: 49) |
| | | 496) | | NO: 180) | | | |
| G8 | G8(1C11) | GTLSS | GWISTYS | CARSYDY | QASQDISN | AASSL | CQQSYSI |
| | | YPIN | GHADYA | GDYLNFD | YLN (SEQ | QS (SEQ | PPTF |
| | | (SEQ ID | (SEQ ID | YW (SEQ | ID NO: 465) | ID NO: | (SEQ ID |
| | | NO: | NO: 525) | ID NO: | | 466) | NO: 197) |
| | | 524) | | 181) | | | |
| G8 | G8(1D02) | FTFSSY | SSISGRG | CARASGS | QASQDISN | AASSL | CQQSYS |
| | | WMS | DNTYYA | GYYYYYG | YLN (SEQ | QS (SEQ | APYTF |
| | | (SEQ ID | (SEQ ID | MDVW | ID NO: 465) | ID NO: | (SEQ ID |
| | | NO: | NO: 527) | (SEQ ID | | 466) | NO: 49) |
| | | 526) | | NO: 182) | | | |
| G8 | G8(1H08) | YTFGN | GMVNPS | CAASTWI | RASQGINS | DASNL | CQQHNS |
| | | YFMH | GGSETFA | QPFDYW | YLA (SEQ | ET (SEQ | YPPTF |
| | | (SEQ ID | (SEQ ID | (SEQ ID | ID NO: 530) | ID NO: | (SEQ ID |
| | | NO: | NO: 529) | NO: 183) | | 516) | NO: 198) |
| | | 528) | | | | | |
| G8 | G8(2B05) | FDFSIY | SAISGSG | CASNGNY | RASQSISR | AASSL | CQQYST |
| | | SMN | GSTYYA | YGSGSYY | WLA (SEQ | QS (SEQ | YPITI |
| | | (SEQ ID | (SEQ ID | NYW (SEQ | ID NO: 533) | ID NO: | (SEQ ID |
| | | NO: | NO: 532) | ID NO: | | 466) | NO: 199) |
| | | 531) | | 184) | | | |
| G8 | G8(2E06) | YTLTT | GWINPN | CARAVYY | RASQGISN | AASSL | CQQANS |
| | | YYMH | SGGTNY | DFWSGPF | SLA (SEQ | QS (SEQ | FPWTF |
| | | (SEQ ID | A (SEQ ID | DYW (SEQ | ID NO: 535) | ID NO: | (SEQ ID |
| | | NO: | NO: 476) | ID NO: | | 466) | NO: 200) |
| | | 534) | | 185) | | | |
| G8 | G8(2C10) | YTFTS | GWINPY | CAKGGIY | RASQDVS | AASSL | CQQSHS |
| | | YYMH | SGGTNY | YGSGSYP | TWLA | QS (SEQ | TPQTF |
| | | (SEQ ID | A (SEQ ID | SW (SEQ | (SEQ ID | ID NO: | (SEQ ID |
| | | NO: | NO: 536) | ID NO: | NO: 537) | 466) | NO: 201) |
| | | 510) | | 186) | | | |
| G8 | G8(2E04) | GTFSSY | GWISPYS | CARGLYY | RASQSISS | DASNL | CQQSYS |
| | | GVS | GNTDYA | MDVW | WLA (SEQ | ET (SEQ | TPLTF |
| | | (SEQ ID | (SEQ ID | (SEQ ID | ID NO: 469) | ID NO: | (SEQ ID |
| | | NO: | NO: 539) | NO: 187) | | 516) | NO: 55) |
| | | 538) | | | | | |
| G8 | G8(4F05) | YTFSN | GWINPN | CARGLYG | RASQGISN | AASTL | CQQSYS |
| | | MYLH | TGDTNY | DYFLYYG | YLA (SEQ | QS (SEQ | TPLTF |
| | | (SEQ ID | A (SEQ ID | MDVW | ID NO: 485) | ID NO: | (SEQ ID |
| | | NO: | NO: 541) | (SEQ ID | | 474) | NO: 55) |
| | | 540) | | NO: 188) | | | |
| G8 | G8(5C03) | YTFTS | GWMNP | CARGLLG | RASQGISN | AASTL | CQQTYS |
| | | YYMH | NSGNTG | FGEFLTY | WLA (SEQ | QS (SEQ | TPWTF |
| | | (SEQ ID | YA (SEQ | GMDVW | ID NO: 542) | ID NO: | (SEQ ID |
| | | NO: | ID NO: | (SEQ ID | | 474) | NO: 202) |
| | | 510) | 511) | NO: 189) | | | |
| G8 | G8(5F02) | YTFTG | GVINPSG | CARDRDS | RASQSVG | GASTR | CQQYGS |
| | | YYIH | GSTTYA | SWTYYYY | NSLA (SEQ | AT | SPYTF |
| | | (SEQ ID | (SEQ ID | GMDVW | ID NO: 545) | (SEQ ID | (SEQ ID |
| | | NO: | NO: 544) | (SEQ ID | | NO: | NO: 203) |
| | | 543) | | NO: 190) | | 546) | |
| G8 | G8(5G08) | YTFTS | GWMNP | CARGLYG | RASQSISG | AASSL | CQQSHS |
| | | NYMH | NSGNTG | DYFLYYG | YLN (SEQ | QS (SEQ | TPLTF |
| | | (SEQ ID | YA (SEQ | MDVW | ID NO: 548) | ID NO: | (SEQ ID |
| | | NO: | ID NO: | (SEQ ID | | 466) | NO: 204) |
| | | 547) | 511) | NO: 188) | | | |
| G8 | G8(1C01) | GTFSSH | GVIIPSG | CARGDYY | RASQNIYT | DASNL | CQQANG |
| | | AIS | GTSYT | DSSGYYF | YLN (SEQ | ET (SEQ | FPLTF |
| | | (SEQ ID | (SEQ ID | PVYFDYW | ID NO: 551) | ID NO: | (SEQ ID |
| | | NO: | NO: 550) | (SEQ ID | | 516) | NO: 205) |
| | | 549) | | NO: 191) | | | |
| G8 | G8(2C11) | YTFTS | GWINPN | CAKDPFW | RASQSISS | AASSL | CQQSYS |
| | | YAMN | SGGTNY | SGHYYYY | YLN (SEQ | QS (SEQ | TPLTF |
| | | (SEQ ID | A (SEQ ID | GMDVW | ID NO: 481) | ID NO: | (SEQ ID |
| | | NO: | NO: 476) | (SEQ ID | | 466) | NO: 55) |
| | | 552) | | NO: 192) | | | |

VH and VL sequences of scFv hits that bind target G10

| Target | Clone | | |
|---|---|---|---|
| group | name | V_{H} | V_{L} |
| G10 | G10(1A07) | EVQLLESGGGLVKPGGSLRLSCAAS | DIQMTQSPSSLSASVGDRVTITCRAS |
| | | GFTFSSYWMSWVRQAPGKGLEWVS | QGISNYLAWYQQKPGKAPKLLIYAAS |
| | | GISARSGRTYYADSVKGRFTISRDDS | SLQGGVPSRFSGSGSGTDFTLTISSL |
| | | KNTLYLQMNSLKTEDTAVYYCARDQ | QPEDFATYYCQQYFTTPYTFGQGTKL |
| | | DTIFGVVITWFDPWGQGTLVTVSS | EIK (SEQ ID NO: 285) |
| | | (SEQ ID NO: 268) | |
| G10 | G10(1B07) | QVQLVQSGAEVKKPGASVKVSCKAS | DIQMTQSPSSLSASVGDRVTITCRAS |
| | | GYTFTSYYMHWVRQAPGQGLEWMG | QSISRWLAWYQQKPGKAPKLLIFDAS |
| | | IIHPGGGTTSYAQKFQGRVTMTRDTS | RLQSGVPSRFSGSGSGTDFTLTISSL |
| | | TSTVYMELSSLRSEDTAVYYCARDKV | QPEDFATYYCQQAEAFPYTFGQGTK |
| | | YGDGFDPWGQGTLVTVSS (SEQ ID | VEIK (SEQ ID NO: 286) |
| | | NO: 269) | |
| G10 | G10(1E12) | QVQLVQSGAEVKKPGASVKVSCKAS | DIQMTQSPSSLSASVGDRVTITCRAS |
| | | GYIFTGYYMHWVRQAPGQGLEWMG | QSISSYLNWYQQKPGKAPKLLIYAAS |
| | | MIGPSDGSTSYAQKFQGRVTMTRDT | SLQSGVPSRFSGSGSGTDFTLTISSL |
| | | STSTVYMELSSLRSEDTAVYYCARED | QPEDFATYYCQQSYSTPITFGQGTRL |
| | | DSMDVWGKGTTVTVSS (SEQ ID NO: | EIK (SEQ ID NO: 287) |
| | | 270) | |
| G10 | G10(1F06) | QVQLVQSGAEVKKPGASVKVSCKAS | DIQMTQSPSSLSASVGDRVTITCRAS |
| | | GYTFIGYYMHWVRQAPGQGLEWMG | QSISNYLNWYQQKPGKAPKLLIYKAS |
| | | MIGPSDGSTSYAQKFQGRVTMTRDT | SLESGVPSRFSGSGSGTDFTLTISSL |
| | | STSTVYMELSSLRSEDTAVYYCARDS | QPEDFATYYCQQSYIIPYTFGQGTKL |
| | | SGLDPWGQGTLVTVSS (SEQ ID NO: | EIK (SEQ ID NO: 288) |
| | | 271) | |
| G10 | G10(1H01) | QVQLVQSGAEVKKPGASVKVSCKAS | DIQMTQSPSSLSASVGDRVTITCRAS |
| | | GYTFTGYYMHWVRQAPGQGLEWMG | QSISNYLNWYQQKPGKAPKLLIYAAS |
| | | MIGPSDGSTSYAQKFQGRVTMTRDT | SLQSGVPSRFSGSGSGTDFTLTISSL |
| | | STSTVYMELSSLRSEDTAVYYCARGV | QPEDFATYYCHQTYSTPLTFGQGTKV |
| | | GNLDYWGQGTLVTVSS (SEQ ID NO: | EIK (SEQ ID NO: 289) |
| | | 272) | |
| G10 | G10(1H08) | QVQLVQSGAEVKKPGASVKVSCKAS | DIQMTQSPSSLSASVGDRVTITCRAS |
| | | GVTFSTSAISWVRQAPGQGLEWMG | QGISNYLAWYQQKPGKAPKLLIYSAS |
| | | WISPYNGNTDYAQMLQGRVTMTRDT | NLQSGVPSRFSGSGSGTDFTLTISSL |
| | | STSTVYMELSSLRSEDTAVYYCARDA | QPEDFATYYCQQAYSFPWTFGQGTK |
| | | HQYYDFWSGYYSGTYYYGMDVWGQ | VEIK (SEQ ID NO: 290) |
| | | GTTVTVSS (SEQ ID NO: 273) | |
| G10 | G10(2C04) | QVQLVQSGAEVKKPGASVKVSCKAS | DIQMTQSPSSLSASVGDRVTITCRAS |
| | | GGTFSNSIINWVRQAPGQGLEWMG | QNISSYLNWYQQKPGKAPKLLIYAAS |
| | | WMNPNSGNTNYAQKFQGRVTMTRD | SLQSGVPSRFSGSGSGTDFTLTISSL |
| | | TSTSTVYMELSSLRSEDTAVYYCARE | QPEDFATYYCQQGYSTPLTFGQGTR |
| | | QWPSYWYFDLWGRGTLVTVSS | LEIK (SEQ ID NO: 291) |
| | | (SEQ ID NO: 274) | |
| G10 | G10(2G11) | QVQLVQSGAEVKKPGASVKVSCKAS | DIQMTQSPSSLSASVGDRVTITCRAS |
| | | GGTFSTHDINWVRQAPGQGLEWMG | QDISRYLAWYQQKPGKAPKLLIYDAS |
| | | VINPSGGSAIYAQKFQGRVTMTRDTS | NLETGVPSRFSGSGSGTDFTLTISSL |
| | | TSTVYMELSSLRSEDTAVYYCARDRG | QPEDFATYYCQQANSFPRTFGQGTK |
| | | YSYGYFDYWGQGTLVTVSS (SEQ ID | VEIK (SEQ ID NO: 292) |
| | | NO: 275) | |
| G10 | G10(3E04) | QVQLVQSGAEVKKPGASVKVSCKAS | DIQMTQSPSSLSASVGDRVTITCQAS |
| | | GNTFIGYYVHWVRQAPGQGLEWVGII | QDISNYLNWYQQKPGKAPKLLIYAAS |
| | | NPNGGSISYAQKFQGRVTMTRDTST | NLQSGVPSRFSGSGSGTDFTLTISSL |
| | | STVYMELSSLRSEDTAVYYCARGSG | QPEDFATYYCQQANSLPYTFGQGTK |
| | | DPNYYYYYGLDVWGQGTTVTVSS | VEIK (SEQ ID NO: 293) |
| | | (SEQ ID NO: 276) | |
| G10 | G10(4A02) | QVQLVQSGAEVKKPGASVKVSCKAS | DIQMTQSPSSLSASVGDRVTITCRAS |
| | | GYTLSYYYMHWVRQAPGQGLEWMG | QSISSYLNWYQQKPGKAPKLLIYAAS |
| | | MIGPSDGSTSYAQRFQGRVTMTRDT | TLQNGVPSRFSGSGSGTDFTLTISSL |
| | | STGTVYMELSSLRSEDTAVYYCARDT | QPEDFATYYCQQSYSTPFTFGPGTK |
| | | GDHFDYWGQGTLVTVSS (SEQ ID | VDIK (SEQ ID NO: 294) |
| | | NO: 277) | |
| G10 | G10(4C05) | QVQLVQSGAEVKKPGASVKVSCKAS | DIQMTQSPSSLSASVGDRVTITCRAS |
| | | GYTFTGYYMHWVRQAPGQGLEWMG | QRISSYLNWYQQKPGKAPKLLIYSAS |
| | | IIGPSDGSTTYAQKFQGRVTMTRDTS | TLQSGVPSRFSGSGSGTDFTLTISSL |
| | | TSTVYMELSSLRSEDTAVYYCARAEN | QPEDFATYYCQQSYSTPFTFGPGTK |
| | | GMDVWGQGTTVTVSS (SEQ ID NO: | VDIK (SEQ ID NO: 295) |
| | | 278) | |
| G10 | G10(4D04) | QVQLVQSGAEVKKPGASVKVSCKAS | DIQMTQSPSSLSASVGDRVTITCRAS |
| | | GYTFTGYYVHWVRQAPGQGLEWMG | QSISSYLAWYQQKPGKAPKLLIYDAS |
| | | IIAPSDGSTNYAQKFQGRVTMTRDTS | KLETGVPSRFSGSGSGTDFTLTISSL |
| | | TSTVYMELSSLRSEDTAVYYCARDPG | QPEDFATYYCQQSYGVPTFGQGTKL |
| | | GYMDVWGKGTTVTVSS (SEQ ID NO: | EIK (SEQ ID NO: 296) |
| | | 279) | |
| G10 | G10(4D10) | QVQLVQSGAEVKKPGASVKVSCKAS | DIQMTQSPSSLSASVGDRVTITCRAS |
| | | GYTFTGYYLHWVRQAPGQGLEWMG | QGISSWLAWYQQKPGKAPKLLIYDAS |
| | | MIGPSDGSTSYAQKFQGRVTMTRDT | NLETGVPSRFSGSGSGTDFTLTISSL |
| | | STSTVYMELSSLRSEDTAVYYCARDG | QPEDFATYYCQQSYSTPLTFGGGTK |
| | | DAFDIWGQGTMVTVSS (SEQ ID NO: | VEIK (SEQ ID NO: 297) |
| | | 280) | |
| G10 | G10(4E07) | QVQLVQSGAEVKKPGSSVKVSCKAS | DIQMTQSPSSLSASVGDRVTITCRAS |
| | | GYTFTGYYMHWVRQAPGQGLEWMG | QSISSYLNWYQQKPGKAPKLLIYAAS |
| | | RISPSDGSTTYAPKFQGRVTITADEST | SLQSGVPSRFSGSGSGTDFTLTISSL |
| | | STAYMELSSLRSEDTAVYYCARDMG | QPEDFATYYCQQSYSTPLTFGGGTK |
| | | DAFDIWGQGTTVTVSS (SEQ ID NO: | VEIK (SEQ ID NO: 176) |
| | | 281) | |
| G10 | G10(4E12) | QVQLVQSGAEVKKPGASVKVSCKAS | DIQMTQSPSSLSASVGDRVTITCRAS |
| | | GYTFTGYYMHWVRQAPGQGLEWMG | QGISTYLAWYQQKPGKAPKLLIYDAS |
| | | MIGPSDGSTSYAQRFQGRVTMTRDT | SLQSGVPSRFSGSGSGTDFTLTISSL |
| | | STSTVYMELSSLRSEDTAVYYCAREE | QPEDFATYYCQQYYSYPWTFGQGTR |
| | | DGMDVWGQGTTVTVSS (SEQ ID NO: | LEIK (SEQ ID NO: 298) |
| | | 282) | |
| G10 | G10(4G06) | QVQLVQSGAEVKKPGASVKVSCKAS | DIQMTQSPSSLSASVGDRVTITCRAS |
| | | GYTLSYYYMHWVRQAPGQGLEWMG | QSISSYLNWYQQKPGKAPKLLIYAAS |
| | | MIGPSDGSTSYAQRFQGRVTMTRDT | TLQNGVPSRFSGSGSGTDFTLTISSL |
| | | STGTVYMELSSLRSEDTAVYYCARDT | QPEDFATYYCQQSYSTPFTFGPGTK |
| | | GDHFDYWGQGTLVTVSS (SEQ ID | VDIK (SEQ ID NO: 294) |
| | | NO: 277) | |
| G10 | G10(5A08) | QVQLVQSGAEVKKPGSSVKVSCKAS | DIVMTQSPLSLPVTPGEPASISCRSSQ |
| | | GGTFNNFAISWVRQAPGQGLEWMG | SLLHSNGYNYLDWYLQKPGQSPQLLI |
| | | GIIPIFDATNYAQKFQGRVTFTADEST | YLGSNRASGVPDRFSGSGSGTDFTL |
| | | STAYMELSSLRSEDTAVYYCARGEYS | KISRVEAEDVGVYYCMQTLKTPLSFG |
| | | SGFFFVGWFDLWGRGTQVTVSS | GGTKVEIK (SEQ ID NO: 299) |
| | | (SEQ ID NO: 283) | |
| G10 | G10(5C08) | QVQLVQSGAEVKKPGASVKVSCKAS | DIQMTQSPSSLSASVGDRVTITCRAS |
| | | GYNFTGYYMHWVRQAPGQGLEWM | QSISSYLNWYQQKPGKAPKLLIYAAS |
| | | GIIAPSDGSTNYAQKFQGRVTMTRDT | SLQSGVPSRFSGSGSGTDFTLTISSL |
| | | STSTVYMELSSLRSEDTAVYYCARET | QPEDFATYYCQQSYSTPLTFGGGTK |
| | | GDDAFDIWGQGTMVTVSS (SEQ ID | VEIK (SEQ ID NO: 176) |
| | | NO: 284) | |

TABLE 9
CDR sequences of identified scFvs to G10, numbered according to the Kabat
scheme

| Target | Clone | | | | | | |
|---|---|---|---|---|---|---|---|
| group | name | HCDR1 | HCDR2 | HCDR3 | LCDR1 | LCDR2 | LCDR3 |
| G10 | G10(1A07) | FTFSSYW | SGISARS | CARDQDTI | RASQGISN | AASSLQ | CQQYFTT |
| | | MS (SEQ | GRTYYA | FGVVITWF | YLA (SEQ | G (SEQ | PYTF |
| | | ID NO: | (SEQ ID | DPW (SEQ | ID NO: 485) | ID NO: | (SEQ ID |
| | | 526) | NO: 553) | ID NO: 239) | | 554) | NO: 256) |
| G10 | G10(1B07) | YTFTSYY | GIIHPGG | CARDKVYG | RASQSISR | DASRLQ | CQQAEAF |
| | | MH (SEQ | GTTSYA | DGFDPW | WLA (SEQ | S (SEQ | PYTF |
| | | ID NO: | (SEQ ID | (SEQ ID | ID NO: 533) | ID NO: | (SEQ ID |
| | | 510) | NO: 555) | NO: 240) | | 556) | NO: 257) |
| G10 | G10(1E12) | YIFTGYYM | GMIGPSD | CAREDDS | RASQSISS | AASSLQ | CQQSYST |
| | | H (SEQ ID | GSTSYA | MDVW | YLN (SEQ | S (SEQ | PITF (SEQ |
| | | NO: 557) | (SEQ ID | (SEQ ID | ID NO: 481) | ID NO: | ID NO: |
| | | | NO: 558) | NO: 241) | | 466) | 258) |
| G10 | G10(1F06) | YTFIGYYM | GMIGPSD | CARDSSGL | RASQSISN | KASSLE | CQQSYIIP |
| | | H (SEQ ID | GSTSYA | DPW (SEQ | YLN (SEQ | S (SEQ | YTF (SEQ |
| | | NO: 559) | (SEQ ID | ID NO: 242) | ID NO: 560) | ID NO: | ID NO: |
| | | | NO: 558) | | | 523) | 259) |
| G10 | G10(1H01) | YTFTGYY | GMIGPSD | CARGVGNL | RASQSISN | AASSLQ | CHQTYST |
| | | MH (SEQ | GSTSYA | DYW (SEQ | YLN (SEQ | S (SEQ | PLTF |
| | | ID NO: | (SEQ ID | ID NO: 243) | ID NO: 560) | ID NO: | (SEQ ID |
| | | 486) | NO: 558) | | | 466) | NO: 260) |
| G10 | G10(1H08) | VTFSTSAI | GWISPYN | CARDAHQ | RASQGISN | SASNLQ | CQQAYSF |
| | | S (SEQ ID | GNTDYA | YYDFWSG | YLA (SEQ | S (SEQ | PWTF |
| | | NO: 561) | (SEQ ID | YYSGTYYY | ID NO: 485) | ID NO: | (SEQ ID |
| | | | NO: 562) | GMDVW | | 563) | NO: 261) |
| | | | | (SEQ ID | | | |
| | | | | NO: 244) | | | |
| G10 | G10(2C04) | GTFSNSII | GWMNPN | CAREQWP | RASQNISS | AASSLQ | CQQGYS |
| | | N (SEQ ID | SGNTNYA | SYWYFDL | YLN (SEQ | S (SEQ | TPLTF |
| | | NO: 564) | (SEQ ID | W (SEQ ID | ID NO: 566) | ID NO: | (SEQ ID |
| | | | NO: 565) | NO: 245) | | 466) | NO: 262) |
| G10 | G10(2G11) | GTFSTHDI | GVINPSG | CARDRGY | RASQDISR | DASNLE | CQQANS |
| | | N (SEQ ID | GSAIYA | SYGYFDY | YLA (SEQ | T (SEQ | FPRTF |
| | | NO: 567) | (SEQ ID | W (SEQ ID | ID NO: 569) | ID NO: | (SEQ ID |
| | | | NO: 568) | NO: 246) | | 516) | NO: 263) |
| G10 | G10(3E04) | NTFIGYYV | GIINPNG | CARGSGD | QASQDISN | AASNLQ | CQQANSL |
| | | H (SEQ ID | GSISYA | PNYYYYYG | YLN (SEQ | S (SEQ | PYTF |
| | | NO: 570) | (SEQ ID | LDVW (SEQ | ID NO: 465) | ID NO: | (SEQ ID |
| | | | NO: 571) | ID NO: 247) | | 572) | NO: 264) |
| G10 | G10(4A02) | YTLSYYY | GMIGPSD | CARDTGD | RASQSISS | AASTLQ | CQQSYST |
| | | MH (SEQ | GSTSYA | HFDYW | YLN (SEQ | N (SEQ | PFTF |
| | | ID NO: | (SEQ ID | (SEQ ID | ID NO: 481) | ID NO: | (SEQ ID |
| | | 573) | NO: 558) | NO: 248) | | 574) | NO: 47) |
| G10 | G10(4C05) | YTFTGYY | GIIGPSDG | CARAENG | RASQRISS | SASTLQ | CQQSYST |
| | | MH (SEQ | STTYA | MDVW | YLN (SEQ | S (SEQ | PFTF |
| | | ID NO: | (SEQ ID | (SEQ ID | ID NO: 576) | ID NO: | (SEQ ID |
| | | 486) | NO: 575) | NO: 249) | | 470) | NO: 47) |
| G10 | G10(4D04) | YTFTGYY | GIIAPSDG | CARDPGG | RASQSISS | DASKLE | CQQSYG |
| | | VH (SEQ | STNYA | YMDVW | YLA (SEQ | T (SEQ | VPTF |
| | | ID NO: | (SEQ ID | (SEQ ID | ID NO: 579) | ID NO: | (SEQ ID |
| | | 577) | NO: 578) | NO: 250) | | 580) | NO: 265) |
| G10 | G10(4D10) | YTFTGYYL | GMIGPSD | CARDGDAF | RASQGISS | DASNLE | CQQSYST |
| | | H (SEQ ID | GSTSYA | DIW (SEQ | WLA (SEQ | T (SEQ | PLTF |
| | | NO: 581) | (SEQ ID | ID NO: 251) | ID NO: 582) | ID NO: | (SEQ ID |
| | | | NO: 558) | | | 516) | NO: 55) |
| G10 | G10(4E07) | YTFTGYY | GRISPSD | CARDMGD | RASQSISS | AASSLQ | CQQSYST |
| | | MH (SEQ | GSTTYA | AFDIW | YLN (SEQ | S (SEQ | PLTF |
| | | ID NO: | (SEQ ID | (SEQ ID | ID NO: 481) | ID NO: | (SEQ ID |
| | | 486) | NO: 583) | NO: 252) | | 466) | NO: 55) |
| G10 | G10(4E12) | YTFTGYY | GMIGPSD | CAREEDG | RASQGIST | DASSLQ | CQQYYS |
| | | MH (SEQ | GSTSYA | MDVW | YLA (SEQ | S (SEQ | YPWTF |
| | | ID NO: | (SEQ ID | (SEQ ID | ID NO: 584) | ID NO: | (SEQ ID |
| | | 486) | NO: 558) | NO: 253) | | 585) | NO: 266) |
| G10 | G10(4G06) | YTLSYYY | GMIGPSD | CARDTGD | RASQSISS | AASTLQ | CQQSYST |
| | | MH (SEQ | GSTSYA | HFDYW | YLN (SEQ | N (SEQ | PFTF |
| | | ID NO: | (SEQ ID | (SEQ ID | ID NO: 481) | ID NO: | (SEQ ID |
| | | 573) | NO: 558) | NO: 248) | | 574) | NO: 47) |
| G10 | G10(5A08) | GTFNNFAI | GGIIPIFD | CARGEYSS | RSSQSLLH | LGSNRA | CMQTLKT |
| | | S (SEQ ID | ATNYA | GFFFVGWF | SNGYNYLD | S (SEQ | PLSF |
| | | NO: 586) | (SEQ ID | DLW (SEQ | (SEQ ID | ID NO: | (SEQ ID |
| | | | NO: 587) | ID NO: 254) | NO: 438) | 505) | NO: 267) |
| G10 | G10(5C08) | YNFTGYY | GIIAPSDG | CARETGDD | RASQSISS | AASSLQ | CQQSYST |
| | | MH (SEQ | STNYA | AFDIW | YLN (SEQ | S (SEQ | PLTF |
| | | ID NO: | (SEQ ID | (SEQ ID | ID NO: 481) | ID NO: | (SEQ ID |
| | | 588) | NO: 578) | NO: 255) | | 466) | NO: 55) |

TABLE 27
VH and VL sequences for G2 scFv Selective Binders, selective for
HLA-PEPTIDE Target HLA-A*01:01_NTDNNLAVY (SEQ ID NO: 5).

| Target | Clone | | |
|---|---|---|---|
| group | name | VH | VL |
| G2 | G2(2E07) | QVQLVQSGAEVKKPGASVKV | DIQMTQSPSSLSASVGDRVTI |
| | | SCKASGGTFSSATISWVRQAP | TCRASQSISTWLAWYQQKPG |
| | | GQGLEWMGWIYPNSGGTVY | KAPKLLIYAASSLRSGVPSRF |
| | | AQKFQGRVTMTRDTSTSTVY | SGSGSGTDFTLTISSLQPEDFA |
| | | MELSSLRSEDTAVYYCAATE | TYYCQQSYNTPYTFGQGTKL |
| | | WLGVWGQGTTVTVSS (SEQ | EIK (SEQ ID NO: 331) |
| | | ID NO: 366) | |
| G2 | G2(2E03) | EVQLLQSGAEVKKPGSSVKV | DIQMTQSPSSLSASVGDRVTI |
| | | SCKASGGTFSSYAISWVRQAP | TCRASQSISRWLAWYQQKPG |
| | | GQGLEWMGWINPNSGGTISA | KAPKLLIYAASTVQSGVPSRF |
| | | PNFQGRVTMTRDTSTSTVYM | SGSGSGTDFTLTISSLQPEDFA |
| | | ELSSLRSEDTAVYYCARANW | TYYCQQSYSTPYTFGQGTKL |
| | | LDYWGQGTLVTVSS (SEQ ID | EIK (SEQ ID NO: 332) |
| | | NO: 367) | |
| G2 | G2(2A11) | EVQLLESGAEVKKPGASVKV | DIQMTQSPSSLSASVGDRVTI |
| | | SCKASGYTFTTYDLAWVRQA | TCRASQDISRWLAWYQQKPG |
| | | PGQGLEWMGWINPNSGGTN | KAPKLLIYAASRLQAGVPSRF |
| | | YAQKFQGRVTMTRDTSTSTV | SGSGSGTDFTLTISSLQPEDFA |
| | | YMELSSLRSEDTAVYYCARA | TYYCQQSYSTPYSFGQGTKLE |
| | | NWLDYWGQGTLVTVSS (SEQ | IK (SEQ ID NO: 333) |
| | | ID NO: 368) | |
| G2 | G2(2C06) | QVQLVQSGAEVKKPGASVKV | DIQMTQSPSSLSASVGDRVTI |
| | | SCKSSGYSFDSYVVNWVRQA | TCRASQTISSWLAWYQQKPG |
| | | PGQGLEWMGWINPNSGGTN | KAPKLLIYAASSLQSGVPSRF |
| | | YAQKFQGRVTMTRDTSTSTV | SGSGSGTDFTLTISSLQPEDFA |
| | | YMELSSLRSEDTAVYYCARD | TYYCQQSYSTPFTFGPGTKVD |
| | | WVLDYWGQGTLVTVSS (SEQ | IK (SEQ ID NO: 334) |
| | | ID NO: 369) | |
| G2 | G2(1G01) | QVQLVQSGAEVKKPGASVKV | DIQMTQSPSSLSASVGDRVTI |
| | | SCKASGYTFTSYGISWVRQAP | TCRASQTISSWLAWYQQKPG |
| | | GQGLEWMGWMNPNSGGTN | KAPKLLIYAASSLQSGVPSRF |
| | | YAQKFQGRVTMTRDTSTSTV | SGSGSGTDFTLTISSLQPEDFA |
| | | YMELSSLRSEDTAVYYCARG | TYYCQQSYGVPYTFGQGTKV |
| | | EWLDYWGQGTLVTVSS (SEQ | EIK (SEQ ID NO: 335) |
| | | ID NO: 370) | |
| G2 | G2(1C02) | QVQLVQSGAEVKKPGASVKV | DIQMTQSPSSLSASVGDRVTI |
| | | SCKASGYTFTSYGISWVRQAP | TCRASQSISNWLAWYQQKPG |
| | | GQGLEWMGWINPNSGGTNY | KAPKLLIYAASSLQSGVPSRF |
| | | AQKFQGRVTMTRDTSTSTVY | SGSGSGTDFTLTISSLQPEDFA |
| | | MELSSLRSEDTAVYYCARGW | TYYCQQSYSAPYTFGPGTKV |
| | | ELGYWGQGTLVTVSS (SEQ | DIK (SEQ ID NO: 336) |
| | | ID NO: 371) | |
| G2 | G2(1H01) | QVQLVQSGAEVKKPGASVKV | DIQMTQSPSSLSASVGDRVTI |
| | | SCKASGYTFTRYTINWVRQA | TCRASQSVGNWLAWYQQKP |
| | | PGQGLEWMGWINPNSGGTN | GKAPKLLIYGASSLQTGVPSR |
| | | YAQKFQGRVTMTRDTSTSTV | FSGSGSGTDFTLTISSLQPEDF |
| | | YMELSSLRSEDTAVYYCARD | ATYYCQQSYSAPYTFGQGTK |
| | | FVGYDDWGQGTLVTVSS | VEIK (SEQ ID NO: 337) |
| | | (SEQ ID NO: 372) | |
| G2 | G2(1B12) | QVQLVQSGAEVKKPGASVKV | DIQMTQSPSSLSASVGDRVTI |
| | | SCKASGYTFTSYGITWVRQAP | TCRASQNIGNWLAWYQQKP |
| | | GQGLEWMGWINPNSGGTNY | GKAPKLLIYAASTLQTGVPSR |
| | | AQKFQGRVTMTRDTSTSTVY | FSGSGSGTDFTLTISSLQPEDF |
| | | MELSSLRSEDTAVYYCARDY | ATYYCQQSYSAPYSFGQGTK |
| | | GDLDYWGQGTLVTVSS (SEQ | LEIK (SEQ ID NO: 338) |
| | | ID NO: 373) | |
| G2 | G2(1B06) | QVQLVQSGAEVKKPGASVKV | DIQMTQSPSSLSASVGDRVTI |
| | | SCKASGGTFSNYILSWVRQAP | TCRASQSISRWLAWYQQKPG |
| | | GQGLEWMGWINPDSGGTNY | KAPKLLIYAASSLQSGVPSRF |
| | | AQKFQGRVTMTRDTSTSTVY | SGSGSGTDFTLTISSLQPEDFA |
| | | MELSSLRSEDTAVYYCARGS | TYYCQQSYSTPYTFGQGTKL |
| | | YGMDVWGQGTTVTVSS | EIK (SEQ ID NO: 339) |
| | | (SEQ ID NO: 374) | |
| G2 | G2(2H10) | QVQLVQSGAEVKKPGASVKV | DIQMTQSPSSLSASVGDRVTI |
| | | SCKASGYSFTRYNMHWVRQ | TCRASQSISSWLAWYQQKPG |
| | | APGQGLEWMGWINPNSGGT | KAPKLLIYGASSLQSGVPSRF |
| | | NYAQKFQGRVTMTRDTSTST | SGSGSGTDFTLTISSLQPEDFA |
| | | VYMELSSLRSEDTAVYYCAR | TYYCQQSYSVPYSFGQGTKL |
| | | DGYSGLDVWGKGTTVTVSS | EIK (SEQ ID NO: 340) |
| | | (SEQ ID NO: 375) | |
| G2 | G2(1H10) | QVQLVQSGAEVKKPGASVKV | DIQMTQSPSSLSASVGDRVTI |
| | | SCKASGGTFSSYAISWVRQAP | TCRASQSISKWLAWYQQKPG |
| | | GQGLEWMGWINPNNGGTNY | KAPKLLIYAASSLQSGVPSRF |
| | | AQKFQGRVTMTRDTSTSTVY | SGSGSGTDFTLTISSLQPEDFA |
| | | MELSSLRSEDTAVYYCARDS | TYYCQQSYSAPYTFGQGTKV |
| | | GVGMDVWGQGTTVTVSS | EIK (SEQ ID NO: 341) |
| | | (SEQ ID NO: 376) | |
| G2 | G2(2C11) | QVQLVQSGAEVKKPGASVKV | DIQMTQSPSSLSASVGDRVTI |
| | | SCKASGGTFNNYAFSWVRQA | TCRASQGISNYLAWYQQKPG |
| | | PGQGLEWMGWINPNSGGTN | KAPKLLIYAASTLQSGVPSRF |
| | | YAQKFQGRVTMTRDTSTSTV | SGSGSGTDFTLTISSLQPEDFA |
| | | YMELSSLRSEDTAVYYCARD | TYYCQQSYSVPYSFGQGTKL |
| | | GVAVASDYWGQGTLVTVSS | EIK (SEQ ID NO: 342) |
| | | (SEQ ID NO: 377) | |
| G2 | G2(1C09) | QVQLVQSGAEVKKPGASVKV | DIQMTQSPSSLSASVGDRVTI |
| | | SCKASGYTFSSYNMHWVRQ | TCRASQTISNYLNWYQQKPG |
| | | APGQGLEWMGWINGNTGGT | KAPKLLIYAASNLQSGVPSRF |
| | | NYAQKFQGRVTMTRDTSTST | SGSGSGTDFTLTISSLQPEDFA |
| | | VYMELSSLRSEDTAVYYCAR | TYYCQQSYSTPQTFGQGTKV |
| | | GVNVDDFDYWGQGTLVTVS | EIK (SEQ ID NO: 343) |
| | | S (SEQ ID NO: 378) | |
| G2 | G2(1A10) | QVQLVQSGAEVKKPGASVKV | DIQMTQSPSSLSASVGDRVTI |
| | | SCKASGGTFSSYAFSWVRQA | TCRASRDIGRAVGWYQQKPG |
| | | PGQGLEWMGWINPDTGYTR | KAPKLLIYAASSLQSGVPSRF |
| | | YAQKFQGRVTMTRDTSTSTV | SGSGSGTDFTLTISSLQPEDFA |
| | | YMELSSLRSEDTAVYYCARG | TYYCQQLDSYPFTFGPGTKV |
| | | DYTGNWYFDLWGRGTLVTV | DIK (SEQ ID NO: 344) |
| | | SS (SEQ ID NO: 379) | |
| G2 | G2(1B10) | EVQLLESGAEVKKPGASVKV | DIQMTQSPSSLSASVGDRVTI |
| | | SCKASGYTFTSYGISWVRQAP | TCRASQSISSWLAWYQQKPG |
| | | GQGLEWMGWINPYSGGTNY | KAPKLLIYAASTLQSGVPSRF |
| | | AQKLQGRVTMTRDTSTSTVY | SGSGSGTDFTLTISSLQPEDFA |
| | | MELSSLRSEDTAVYYCARAN | TYYCQQSYSSPYTFGPGTKV |
| | | WLDYWGQGTL VTVSS (SEQ | DIK (SEQ ID NO: 345) |
| | | ID NO: 380) | |
| G2 | G2(1D07) | QVQLVQSGAEVKKPGASVKV | DIQMTQSPSSLSASVGDRVTI |
| | | SCKASGYTFTSYGISWVRQAP | TCQASQDISNYLNWYQQKPG |
| | | GQGLEWMGWISAYNGYTNY | KAPKLLIYAASSLQSGVPSRF |
| | | AQNLQGRVTMTRDTSTSTVY | SGSGSGTDFTLTISSLQPEDFA |
| | | MELSSLRSEDTAVYYCARDQ | TYYCQQSYSTPLTFGGGTKLE |
| | | FYGGNSGGHDYWGQGTLVT | IK (SEQ ID NO: 346) |
| | | VSS (SEQ ID NO: 381) | |
| G2 | G2(1E05) | QVQLVQSGAEVKKPGASVKV | DIQMTQSPSSLSASVGDRVTI |
| | | SCKASGYTFTDYNMHWVRQ | TCRASQSIGRWLAWYQQKPG |
| | | APGQGLEWMGWMNPNSGGT | KAPKLLIYAASSLQSGVPSRF |
| | | NYAQKFQGRVTMTRDTSTST | SGSGSGTDFTLTISSLQPEDFA |
| | | VYMELSSLRSEDTAVYYCAR | TYYCQQSYSTPYSFGQGTKV |
| | | E-EDYWGQGTLVTVSS (SEQ | EIK (SEQ ID NO: 347) |
| | | ID NO: 382) | |
| G2 | G2(1D03) | QVQLVQSGAEVKKPGASVKV | DIQMTQSPSSLSASVGDRVTI |
| | | SCKASGYTFTRYTINWVRQA | TCRASQSISTWLAWYQQKPG |
| | | PGQGLEWMGWINPNSGGAN | KAPKLLIYAASTLQSGVPSRF |
| | | YAQKFQGRVTMTRDTSTSTV | SGSGSGTDFTLTISSLQPEDFA |
| | | YMELSSLRSEDTAVYYCARG | TYYCQQSYSTPYTFAQGTKL |
| | | DWFDPWGQGTLVTVSS (SEQ | EIK (SEQ ID NO: 348) |
| | | ID NO: 383) | |
| G2 | G2(1G12) | QVQLVQSGAEVKKPGASVKV | DIQMTQSPSSLSASVGDRVTI |
| | | SCKASGYTFTSYLMHWVRQA | TCQASQDISNYLNWYQQKPG |
| | | PGQGLEWMGWISPNSGGTNY | KAPKLLIYGASRLQSGVPSRF |
| | | AQKFQGRVTMTRDTSTSTVY | SGSGSGTDFTLTISSLQPEDFA |
| | | MELSSLRSEDTAVYYCARGD | TYYCQQSYSTPYTFGQGTKL |
| | | WFDPWGQGTLVTVSS (SEQ | EIK (SEQ ID NO: 349) |
| | | ID NO: 384) | |
| G2 | G2(2H11) | QVQLVQSGAEVKKPGASVKV | DIQMTQSPSSLSASVGDRVTI |
| | | SCKASGYTFSDYYVHWVRQ | TCRASQSISSWLAWYQQKPG |
| | | APGQGLEWMGWINPNSGGT | KAPKLLIYAASTLQSGVPSRF |
| | | NYAQKFQGRVTMTRDTSTST | SGSGSGTDFTLTISSLQPEDFA |
| | | VYMELSSLRSEDTAVYYCAR | TYYCQQSYSTPFTFGPGTKVD |
| | | GEWFDPWGQGTLVTVSS | IK (SEQ ID NO: 350) |
| | | (SEQ ID NO: 385) | |
| G2 | G2(1C03) | QVQLVQSGAEVKKPGASVKV | DIQMTQSPSSLSASVGDRVTI |
| | | SCKASGYTFTTYYMHWVRQ | TCRASQSVSNWLAWYQQKP |
| | | APGQGLEWMGWINPNSGGT | GKAPKLLIYAASSLQSGVPSR |
| | | NYAQKFQGRVTMTRDTSTST | FSGSGSGTDFTLTISSLQPEDF |
| | | VYMELSSLRSEDTAVYYCAR | ATYYCQQSYSTPTFGQGTKL |
| | | SDWFDPWGQGTLVTVSS | EIK (SEQ ID NO: 351) |
| | | (SEQ ID NO: 386) | |
| G2 | G2(1G07) | QVQLVQSGAEVKKPGASVKV | DIQMTQSPSSLSASVGDRVTI |
| | | SCKASGGTFSNYAINWVRQA | TCQASQDISNYLNWYQQKPG |
| | | PGQGLEWMGWISPYSGGTNY | KAPKLLIYAASTLQSGVPSRF |
| | | AQKFQGRVTMTRDTSTSTVY | SGSGSGTDFTLTISSLQPEDFA |
| | | MELSSLRSEDTAVYYCARDS | TYYCQQTYAIPLTFGGGTKVE |
| | | GSYFDYWGQGTLVTVSS | IK (SEQ ID NO: 352) |
| | | (SEQ ID NO: 387) | |
| G2 | G2(1F12) | QVQLVQSGAEVKKPGASVKV | DIQMTQSPSSLSASVGDRVTI |
| | | SCKASGYTFTDYYMHWVRQ | TCQASQDIGSWLAWYQQKPG |
| | | APGQGLEWMGWIYPNTGGT | KAPKLLIYATSSLQSGVPSRFS |
| | | NYAQKFQGRVTMTRDTSTST | GSGSGTDFTLTISSLQPEDFAT |
| | | VYMELSSLRSEDTAVYYCAR | YYCQQSYSTPYTFGQGTKLEI |
| | | DYGGYVDYWGQGTLVTVSS | K (SEQ ID NO: 353) |
| | | (SEQ ID NO: 388) | |
| G2 | G2(1G03) | EVQLLESGAEVKKPGASVKV | DIQMTQSPSSLSASVGDRVTI |
| | | SCKASGYTFTSYAMNWVRQ | TCRASQGISRWLAWYQQKPG |
| | | APGQGLEWMGWMNPNSGGT | KAPKLLIYAASTLQPGVPSRF |
| | | KYAQKFQGRVTMTRDTSTST | SGSGSGTDFTLTISSLQPEDFA |
| | | VYMELSSLRSEDTAVYYCAR | TYYCQQSYIAPFTFGPGTKVD |
| | | EGPAALDVWGQGTL VTVSS | IK (SEQ ID NO: 354) |
| | | (SEQ ID NO: 389) | |
| G2 | G2(2B08) | QVQLVQSGAEVKKPGASVKV | DIQMTQSPSSLSASVGDRVTI |
| | | SCKASGYTLTSHLIHWVRQA | TCRASQGISNYLAWYQQKPG |
| | | PGQGLEWMGWINPNSGGTN | KAPKLLIYAASRLESGVPSRF |
| | | YAQKFQGRVTMTRDTSTSTV | SGSGSGTDFTLTISSLQPEDFA |
| | | YMELSSLRSEDTAVYYCARE | TYYCQQSYSIPLTFGGGTKVE |
| | | RRSGMDVWGQGTTVTVSS | IK (SEQ ID NO: 355) |
| | | (SEQ ID NO: 390) | |
| G2 | G2(2A10) | EVQLLESGAEVKKPGASVKV | DIQMTQSPSSLSASVGDRVTI |
| | | SCKASGYSFTDYIVHWVRQA | TCRASQSISSYLNWYQQKPG |
| | | PGQGLEWMGWINPYSGGTK | KAPKLLIYGVSSLQSGVPSRF |
| | | YAQKFQGRVTMTRDTSTSTV | SGSGSGTDFTLTISSLQPEDFA |
| | | YMELSSLRSEDTAVYYCARV | TYYCQQSYSNPTFGQGTKVEI |
| | | LQEGMDVWGQGTLVTVSS | K (SEQ ID NO: 356) |
| | | (SEQ ID NO: 391) | |
| G2 | G2(2D04) | QVQLVQSGAEVKKPGASVKV | DIQMTQSPSSLSASVGDRVTI |
| | | SCKASGYTFSNFLINWVRQAP | TCRASQSISSWVAWYQQKPG |
| | | GQGLEWMGWINPNSGGTNY | KAPKLLIYGASNLESGVPSRF |
| | | AQKFQGRVTMTRDTSTSTVY | SGSGSGTDFTLTISSLQPEDFA |
| | | MELSSLRSEDTAVYYCASERE | TYYCQQSYSTPYSFGQGTKLE |
| | | LPFDIWGQGTMVTVSS (SEQ | IK (SEQ ID NO: 357) |
| | | ID NO: 392) | |
| G2 | G2(1C06) | QVQLVQSGAEVKKPGASVKV | DIQMTQSPSSLSASVGDRVTI |
| | | SCKASGYTFTDYQMFWVRQ | TCRASQGISNYLAWYQQKPG |
| | | APGQGLEWMGWINPNSGGT | KAPKLLIYAASSLQSGVPSRF |
| | | NYAQKFQGRVTMTRDTSTST | SGSGSGTDFTLTISSLQPEDFA |
| | | VYMELSSLRSEDTAVYYCAK | TYYCQQSYSDQWTFGQGTK |
| | | GGGGYGMDVWGQGTTVTVS | VEIK (SEQ ID NO: 358) |
| | | S (SEQ ID NO: 393) | |
| G2 | G2(2A09) | QVQLVQSGAEVKKPGASVKV | DIQMTQSPSSLSASVGDRVTI |
| | | SCKASGGTFSSYAISWVRQAP | TCRASQSISRWLAWYQQKPG |
| | | GQGLEWMGWINPNSGGTNY | KAPKLLIYAASSLQSGVPSRF |
| | | AQKFQGRVTMTRDTSTSTVY | SGSGSGTDFTLTISSLQPEDFA |
| | | MELSSLRSEDTAVYYCAAMG | TYYCQQSYLPPYSFGQGTKV |
| | | IAVAGGMDVWGQGTLVTVS | EIK (SEQ ID NO: 359) |
| | | S (SEQ ID NO: 394) | |
| G2 | G2(1B08) | QVQLVQSGAEVKKPGASVKV | DIQMTQSPSSLSASVGDRVTI |
| | | SCKASGYTFTNYHMHWVRQ | TCRASQSISNWLAWYQQKPG |
| | | APGQGLEWMGWIHPDSGGTS | KAPKLLIYAASSLQSGVPSRF |
| | | YAQKFQGRVTMTRDTSTSTV | SGSGSGTYFTLTISSLQPEDFA |
| | | YMELSSLRSEDTAVYYCARN | TYYCQQSYSSPYTFGQGTKLE |
| | | WNLDYWGQGTLVTVSS (SEQ | IK (SEQ ID NO: 360) |
| | | ID NO: 395) | |
| G2 | G2(1E03) | QVQLVQSGAEVKKPGASVKV | DIQMTQSPSSLSASVGDRVTI |
| | | SCKASGYTFTGYYMHWVRQ | TCRASQSISHYLNWYQQKPG |
| | | APGQGLEWMGWMNPNSGNT | KAPKLLIYGASSLQSGVPSRF |
| | | GYAQKFQGRVTMTRDTSTST | SGSGSGTDFTLTISSLQPEDFA |
| | | VYMELSSLRSEDTAVYYCAT | TYYCQQSYTTPWTFGQGTRL |
| | | YDDGMDVWGQGTTVTVSS | EIK (SEQ ID NO: 361) |
| | | (SEQ ID NO: 396) | |
| G2 | G2(2A03) | QVQLVQSGAEVKKPGASVKV | DIQMTQSPSSLSASVGDRVTI |
| | | SCKASGYTFTSYTVNWVRQA | TCRASQSISSWLAWYQQKPG |
| | | PGQGLEWMGWINPNSGGTK | KAPKLLIYAASTLQSGVPSRF |
| | | YAQNFQGRVTMTRDTSTSTV | SGSGSGTDFTLTISSLQPEDFA |
| | | YMELSSLRSEDTAVYYCARG | TYYCQQSYLPPYSFGQGTKLE |
| | | GGGALDYWGQGTLVTVSS | IK (SEQ ID NO: 362) |
| | | (SEQ ID NO: 397) | |
| G2 | G2(2F01) | QVQLVQSGAEVKKPGASVKV | DIQMTQSPSSLSASVGDRVTI |
| | | SCKASGYTFTSYYMHWVRQ | TCQASQDISNYLNWYQQKPG |
| | | APGQGLEWMGMINPRDDTT | KAPKLLIYGASRLQSGVPSRF |
| | | DYARDFQGRVTMTRDTSTST | SGSGSGTDFTLTISSLQPEDFA |
| | | VYMELSSLRSEDTAVYYCAL | TYYCQEGITYTFGQGTKVEIK |
| | | SGNYYGMDVWGQGTTVTVS | (SEQ ID NO: 363) |
| | | S (SEQ ID NO: 398) | |
| G2 | G2(1H11) | QVQLVQSGAEVKKPGASVKV | DIQMTQSPSSLSASVGDRVTI |
| | | SCKASGYTFTNYYMHWVRQ | TCQASQDISNYLNWYQQKPG |
| | | APGQGLEWMGMINPSGGGTS | KAPKLLIYAASSLQSGVPSRF |
| | | YAQKFQGRVTMTRDTSTSTV | SGSGSGTDFTLTISSLQPEDFA |
| | | YMELSSLRSEDTAVYYCARG | TYYCQQYYSYPFTFGPGTKV |
| | | NPWELRLDYWGQGTLVTVSS | DIK (SEQ ID NO: 364) |
| | | (SEQ ID NO: 399) | |
| G2 | G2(1D06) | QVQLVQSGAEVKKPGSSVKV | EIVMTQSPATLSVSPGERATL |
| | | SCKASGYTFTSQYMHWVRQ | SCRASQSVSRNLAWYQQKPG |
| | | APGQGLEWMGRIIPLLGIVNY | QAPRLLIYGASTRATGIPARFS |
| | | AQKFQGRVTITADESTSTAY | GSGSGTEFTLTISSLQSEDFAV |
| | | MELSSLRSEDTAVYYCARDK | YYCQHYGYSPVTFGQGTKLE |
| | | NYYGMDVWGQGTTVTVSS | IK (SEQ ID NO: 365) |
| | | (SEQ ID NO: 400) | |

TABLE 28
CDR sequences for G2 selective binders, selective for HLA-PEPTIDE
Target HLA-A*01:01_NTDNNLAVY (SEQ ID NO: 5) (determined according to
Kabat numbering)

| Target | Clone | | | | | | |
|---|---|---|---|---|---|---|---|
| group | name | CDR-H1 | CDR-H2 | CDR-H3 | CDR-L1 | CDR-L2 | CDR-L3 |
| G2 | G2(2E07) | GTFSSA | GWIYPN | CAATE | RASQSI | AASSLR | CQQSYN |
| | | TIS (SEQ | SGGTVY | WLGVW | STWLA | S (SEQ | TPYTF |
| | | ID NO: | A (SEQ | (SEQ ID | (SEQ ID | ID NO: | (SEQ ID |
| | | 589) | ID NO: | NO: 12) | NO: 591) | 592) | NO: 44) |
| | | | 590) | | | | |
| G2 | G2(2E03) | GTFSSY | GWINPN | CARAN | RASQSI | AASTV | CQQSYS |
| | | AIS (SEQ | SGGTIS | WLDYW | SRWLA | QS (SEQ | TPYTF |
| | | ID NO: | A (SEQ | (SEQ ID | (SEQ ID | ID NO: | (SEQ ID |
| | | 500) | ID NO: | NO: 13) | NO: 533) | 594) | NO: 45) |
| | | | 593) | | | | |
| G2 | G2(2A11) | YTFTTY | GWINPN | CARAN | RASQDI | AASRLQ | CQQSYS |
| | | DLA | SGGTNY | WLDYW | SRWLA | A (SEQ | TPYSF |
| | | (SEQ ID | A (SEQ | (SEQ ID | (SEQ ID | ID NO: | (SEQ ID |
| | | NO: 595) | ID NO: | NO: 13) | NO: 596) | 597) | NO: 46) |
| | | | 476) | | | | |
| G2 | G2(2C06) | YSFDSY | GWINPN | CARDW | RASQTI | AASSLQ | CQQSYS |
| | | VVN | SGGTNY | VLDYW | SSWLA | S (SEQ | TPFTF |
| | | (SEQ ID | A (SEQ | (SEQ ID | (SEQ ID | ID NO: | (SEQ ID |
| | | NO: 598) | ID NO: | NO: 14) | NO: 599) | 466) | NO: 47) |
| | | | 476) | | | | |
| G2 | G2(1G01) | YTFTSY | GWMNP | CARGE | RASQTI | AASSLQ | CQQSYG |
| | | GIS (SEQ | NSGGTN | WLDYW | SSWLA | S (SEQ | VPYTF |
| | | ID NO: | YA (SEQ | (SEQ ID | (SEQ ID | ID NO: | (SEQ ID |
| | | 600) | ID NO: | NO: 15) | NO: 599) | 466) | NO: 48) |
| | | | 601) | | | | |
| G2 | G2(1C02) | YTFTSY | GWINPN | CARGW | RASQSI | AASSLQ | CQQSYS |
| | | GIS (SEQ | SGGTNY | ELGYW | SNWLA | S (SEQ | APYTF |
| | | ID NO: | A (SEQ | (SEQ ID | (SEQ ID | ID NO: | (SEQ ID |
| | | 600) | ID NO: | NO: 16) | NO: 602) | 466) | NO: 49) |
| | | | 476) | | | | |
| G2 | G2(1H01) | YTFTRY | GWINPN | CARDFV | RASQSV | GASSLQ | CQQSYS |
| | | TIN | SGGTNY | GYDDW | GNWLA | T (SEQ | APYTF |
| | | (SEQ ID | A (SEQ | (SEQ ID | (SEQ ID | ID NO: | (SEQ ID |
| | | NO: 603) | ID NO: | NO: 17) | NO: 604) | 605) | NO: 49) |
| | | | 476) | | | | |
| G2 | G2(1B12) | YTFTSY | GWINPN | CARDY | RASQNI | AASTLQ | CQQSYS |
| | | GIT | SGGTNY | GDLDY | GNWLA | T (SEQ | APYSF |
| | | (SEQ ID | A (SEQ | W (SEQ | (SEQ ID | ID NO: | (SEQ ID |
| | | NO: 606) | ID NO: | ID NO: | NO: 607) | 608) | NO: 50) |
| | | | 476) | 18) | | | |
| G2 | G2(1B06) | GTFSNY | GWINPD | CARGSY | RASQSI | AASSLQ | CQQSYS |
| | | ILS (SEQ | SGGTNY | GMDVW | SRWLA | S (SEQ | TPYTF |
| | | ID NO: | A (SEQ | (SEQ ID | (SEQ ID | ID NO: | (SEQ ID |
| | | 609) | ID NO: | NO: 19) | NO: 533) | 466) | NO: 45) |
| | | | 610) | | | | |
| G2 | G2(2H10) | YSFTRY | GWINPN | CARDG | RASQSI | GASSLQ | CQQSYS |
| | | NMH | SGGTNY | YSGLDV | SSWLA | S (SEQ | VPYSF |
| | | (SEQ ID | A (SEQ | W (SEQ | (SEQ ID | ID NO: | (SEQ ID |
| | | NO: 611) | ID NO: | ID NO: | NO: 469) | 482) | NO: 51) |
| | | | 476) | 20) | | | |
| G2 | G2(1H10) | GTFSSY | GWINPN | CARDSG | RASQSI | AASSLQ | CQQSYS |
| | | AIS (SEQ | NGGTN | VGMDV | SKWLA | S (SEQ | APYTF |
| | | ID NO: | YA (SEQ | W (SEQ | (SEQ ID | ID NO: | (SEQ ID |
| | | 500) | ID NO: | ID NO: | NO: 613) | 466) | NO: 49) |
| | | | 612) | 21) | | | |
| G2 | G2(2C11) | GTFNNY | GWINPN | CARDG | RASQGI | AASTLQ | CQQSYS |
| | | AFS | SGGTNY | VAVAS | SNYLA | S (SEQ | VPYSF |
| | | (SEQ ID | A (SEQ | DYW | (SEQ ID | ID NO: | (SEQ ID |
| | | NO: 614) | ID NO: | (SEQ ID | NO: 485) | 474) | NO: 51) |
| | | | 476) | NO: 22) | | | |
| G2 | G2(1C09) | YTFSSY | GWING | CARGV | RASQTI | AASNL | CQQSYS |
| | | NMH | NTGGT | NVDDF | SNYLN | QS (SEQ | TPQTF |
| | | (SEQ ID | NYA | DYW | (SEQ ID | ID NO: | (SEQ ID |
| | | NO: 615) | (SEQ ID | (SEQ ID | NO: 617) | 572) | NO: 52) |
| | | | NO: 616) | NO: 23) | | | |
| G2 | G2(1A10) | GTFSSY | GWINPD | CARGD | RASRDI | AASSLQ | CQQLDS |
| | | AFS | TGYTRY | YTGNW | GRAVG | S (SEQ | YPFTF |
| | | (SEQ ID | A (SEQ | YFDLW | (SEQ ID | ID NO: | (SEQ ID |
| | | NO: 618) | ID NO: | (SEQ ID | NO: 620) | 466) | NO: 53) |
| | | | 619) | NO: 24) | | | |
| G2 | G2(1B10) | YTFTSY | GWINPY | CARAN | RASQSI | AASTLQ | CQQSYS |
| | | GIS (SEQ | SGGTNY | | SSWLA | S (SEQ | SPYTF |
| | | ID NO: | A (SEQ | WLDYW | (SEQ ID | ID NO: | (SEQ ID |
| | | 600) | ID NO: | (SEQ ID | NO: 469) | 474) | NO: 54) |
| | | | 536) | NO: 13) | | | |
| G2 | G2(1D07) | YTFTSY | GWISAY | CARDQF | QASQDI | AASSLQ | CQQSYS |
| | | GIS (SEQ | NGYTN | YGGNS | SNYLN | S (SEQ | TPLTF |
| | | ID NO: | YA (SEQ | GGHDY | (SEQ ID | ID NO: | (SEQ ID |
| | | 600) | ID NO: | W (SEQ | NO: 465) | 466) | NO: 55) |
| | | | 621) | ID NO: | | | |
| | | | | 25) | | | |
| G2 | G2(1E05) | YTFTDY | GWMNP | CAREED | RASQSI | AASSLQ | CQQSYS |
| | | NMH | NSGGTN | YW | GRWLA | S (SEQ | TPYSF |
| | | (SEQ ID | YA (SEQ | (SEQ ID | (SEQ ID | ID NO: | (SEQ ID |
| | | NO: 622) | ID NO: | NO: 26) | NO: 623) | 466) | NO: 46) |
| | | | 601) | | | | |
| G2 | G2(1D03) | YTFTRY | GWINPN | CARGD | RASQSI | AASTLQ | CQQSYS |
| | | TIN | SGGAN | WFDPW | STWLA | S (SEQ | TPYTF |
| | | (SEQ ID | YA (SEQ | (SEQ ID | (SEQ ID | ID NO: | (SEQ ID |
| | | NO: 603) | ID NO: | NO: 27) | NO: 591) | 474) | NO: 45) |
| | | | 624) | | | | |
| G2 | G2(1G12) | YTFTSY | GWISPN | CARGD | QASQDI | GASRLQ | CQQSYS |
| | | LMH | SGGTNY | WFDPW | SNYLN | S (SEQ | TPYTF |
| | | (SEQ ID | A (SEQ | (SEQ ID | (SEQ ID | ID NO: | (SEQ ID |
| | | NO: 625) | ID NO: | NO: 27) | NO: 465) | 502) | NO: 45) |
| | | | 626) | | | | |
| G2 | G2(2H11) | YTFSDY | GWINPN | CARGE | RASQSI | AASTLQ | CQQSYS |
| | | YVH | SGGTNY | WFDPW | SSWLA | S (SEQ | TPFTF |
| | | (SEQ ID | A (SEQ | (SEQ ID | (SEQ ID | ID NO: | (SEQ ID |
| | | NO: 627) | ID NO: | NO: 28) | NO: 469) | 474) | NO: 47) |
| | | | 476) | | | | |
| G2 | G2(1C03) | YTFTTY | GWINPN | CARSD | RASQSV | AASSLQ | CQQSYS |
| | | YMH | SGGTNY | WFDPW | SNWLA | S (SEQ | TPTF |
| | | (SEQ ID | A (SEQ | (SEQ ID | (SEQ ID | ID NO: | (SEQ ID |
| | | NO: 628) | ID NO: | NO: 29) | NO: 629) | 466) | NO: 56) |
| | | | 476) | | | | |
| G2 | G2(1G07) | GTFSNY | GWISPY | CARDSG | QASQDI | AASTLQ | CQQTY |
| | | AIN | SGGTNY | SYFDY | SNYLN | S (SEQ | AIPLTF |
| | | (SEQ ID | A (SEQ | W (SEQ | (SEQ ID | ID NO: | (SEQ ID |
| | | NO: 630) | ID NO: | ID NO: | NO: 465) | 474) | NO: 57) |
| | | | 631) | 30) | | | |
| G2 | G2(1F12) | YTFTDY | GWIYPN | CARDY | QASQDI | ATSSLQ | CQQSYS |
| | | YMH | TGGTN | GGYVD | GSWLA | S (SEQ | TPYTF |
| | | (SEQ ID | YA (SEQ | YW | (SEQ ID | ID NO: | (SEQ ID |
| | | NO: 632) | ID NO: | (SEQ ID | NO: 634) | 635) | NO: 45) |
| | | | 633) | NO: 31) | | | |
| G2 | G2(1G03) | YTFTSY | GWMNP | CAREGP | RASQGI | AASTLQ | CQQSYI |
| | | AMN | NSGGTK | AALDV | SRWLA | P (SEQ | APFTF |
| | | (SEQ ID | YA (SEQ | W (SEQ | (SEQ ID | ID NO: | (SEQ ID |
| | | NO: 552) | ID NO: | ID NO: | NO: 637) | 638) | NO: 58) |
| | | | 636) | 32) | | | |
| G2 | G2(2B08) | YTLTSH | GWINPN | CARERR | RASQGI | AASRLE | CQQSYS |
| | | LIH | SGGTNY | SGMDV | SNYLA | S (SEQ | IPL TF |
| | | (SEQ ID | A (SEQ | W (SEQ | (SEQ ID | ID NO: | (SEQ ID |
| | | NO: 639) | ID NO: | ID NO: | NO: 485) | 640) | NO: 59) |
| | | | 476) | 33) | | | |
| G2 | G2(2A10) | YSFTDY | GWINPY | CARVL | RASQSI | GVSSLQ | CQQSYS |
| | | IVH | SGGTKY | QEGMD | SSYLN | S (SEQ | NPTF |
| | | (SEQ ID | A (SEQ | VW | (SEQ ID | ID NO: | (SEQ ID |
| | | NO: 641) | ID NO: | (SEQ ID | NO: 481) | 643) | NO: 60) |
| | | | 642) | NO: 34) | | | |
| G2 | G2(2D04) | YTFSNF | GWINPN | CASERE | RASQSI | GASNLE | CQQSYS |
| | | LIN | SGGTNY | LPFDIW | SSWVA | S (SEQ | TPYSF |
| | | (SEQ ID | A (SEQ | (SEQ ID | (SEQ ID | ID NO: | (SEQ ID |
| | | NO: 644) | ID NO: | NO: 35) | NO: 645) | 646) | NO: 46) |
| | | | 476) | | | | |
| G2 | G2(1C06) | YTFTDY | GWINPN | CAKGG | RASQGI | AASSLQ | CQQSYS |
| | | QMF | SGGTNY | GGYGM | SNYLA | S (SEQ | DQWTF |
| | | (SEQ ID | A (SEQ | DVW | (SEQ ID | ID NO: | (SEQ ID |
| | | NO: 647) | ID NO: | (SEQ ID | NO: 485) | 466) | NO: 61) |
| | | | 476) | NO: 36) | | | |
| G2 | G2(2A09) | GTFSSY | GWINPN | CAAMGI | RASQSI | AASSLQ | CQQSYL |
| | | AIS (SEQ | SGGTNY | AVAGG | SRWLA | S (SEQ | PPYSF |
| | | ID NO: | A (SEQ | MDVW | (SEQ ID | ID NO: | (SEQ ID |
| | | 500) | ID NO: | (SEQ ID | NO: 533) | 466) | NO: 62) |
| | | | 476) | NO: 37) | | | |
| G2 | G2(1B08) | YTFTNY | GWIHPD | CARNW | RASQSI | AASSLQ | CQQSYS |
| | | HMH | SGGTSY | NLDYW | SNWLA | S (SEQ | SPYTF |
| | | (SEQ ID | A (SEQ | (SEQ ID | (SEQ ID | ID NO: | (SEQ ID |
| | | NO: 648) | ID NO: | NO: 38) | NO: 602) | 466) | NO: 54) |
| | | | 649) | | | | |
| G2 | G2(1E03) | YTFTGY | GWMNP | CATYD | RASQSI | GASSLQ | CQQSYT |
| | | YMH | NSGNTG | DGMDV | SHYLN | S (SEQ | TPWTF |
| | | (SEQ ID | YA (SEQ | W (SEQ | (SEQ ID | ID NO: | (SEQ ID |
| | | NO: 486) | ID NO: | ID NO: | NO: 650) | 482) | NO: 63) |
| | | | 511) | 39) | | | |
| G2 | G2(2A03) | YTFTSY | GWINPN | CARGG | RASQSI | AASTLQ | CQQSYL |
| | | TVN | SGGTKY | GGALD | SSWLA | S (SEQ | PPYSF |
| | | (SEQ ID | A (SEQ | YW | (SEQ ID | ID NO: | (SEQ ID |
| | | NO: 651) | ID NO: | (SEQ ID | NO: 469) | 474) | NO: 62) |
| | | | 652) | NO: 40) | | | |
| G2 | G2(2F01) | YTFTSY | GMINPR | CALSGN | QASQDI | GASRLQ | CQEGIT |
| | | YMH | DDTTD | YYGMD | SNYLN | S (SEQ | YTF |
| | | (SEQ ID | YA (SEQ | VW | (SEQ ID | ID NO: | (SEQ ID |
| | | NO: 510) | ID NO: | (SEQ ID | NO: 465) | 502) | NO: 64) |
| | | | 653) | NO: 41) | | | |
| G2 | G2(1H11) | YTFTNY | GMINPS | CARGNP | QASQDI | AASSLQ | CQQYYS |
| | | YMH | GGGTSY | WELRL | SNYLN | S (SEQ | YPFTF |
| | | (SEQ ID | A (SEQ | DYW | (SEQ ID | ID NO: | (SEQ ID |
| | | NO: 506) | ID NO: | (SEQ ID | NO: 465) | 466) | NO: 65) |
| | | | 654) | NO: 42) | | | |
| G2 | G2(1D06) | YTFTSQ | GRIIPLL | CARDK | RASQSV | GASTRA | CQHYG |
| | | YMH | | NYYGM | SRNLA | T (SEQ | YSPVTF |
| | | (SEQ ID | GIVNYA | DVW | (SEQ ID | ID NO: | (SEQ ID |
| | | NO: 655) | (SEQ ID | (SEQ ID | NO: 657) | 546) | NO: 66) |
| | | | NO: 656) | NO: 43) | | | |

TABLE 29
VH and VL sequences for scFv selective binders selective for HLA-
PEPTIDE Target HLA-A*02:01_LLASSILCA (SEQ ID NO: 8).

| Target | Clone | | |
|---|---|---|---|
| group | name | VH | VL |
| G7 | G7(1C06) | QVQLVQSGAEVKKPGASVKV | EIVMTQSPATLSVSPGERATL |
| | | SCKASGGTFSNYGISWVRQAP | SCRASQSVSSSNLAWYQQKP |
| | | GQGLEWMGIINPGGSTSYAQK | GQAPRLLIYGASTRATGIPAR |
| | | FQGRVTMTRDTSTSTVYMELS | FSGSGSGTEFTLTISSLQSEDF |
| | | SLRSEDTAVYYCARDGYDFW | AVYYCHHYGRSHTFGQGTKV |
| | | SGYTSDDYWGQGTLVTVSS | EIK (SEQ ID NO: 315) |
| | | (SEQ ID NO: 323) | |
| G7 | G7(1G10) | EVQLLESGGGLVQPGGSLRLS | DIQMTQSPSSLSASVGDRVTIT |
| | | CAASGFTFSSYAMHWVRQAP | CRASQDIRNDLGWYQQKPGK |
| | | GKGLEWVSGISGSGGSTYYAD | APKLLIYAASSLQSGVPSRFSG |
| | | SVKGRFTISRDNSKNTLYLQM | SGSGTDFTLTISSLQPEDFATY |
| | | NSLRAEDTAVYYCASDYGDY | YCQQANAFPPTFGQGTKVEIK |
| | | RGQGTLVTVSS (SEQ ID NO: | (SEQ ID NO: 316) |
| | | 324) | |
| G7 | G7(1B04) | QVQLVQSGAEVKKPGASVKV | DIVMTQSPDSLAVSLGERATI |
| | | SCKASGYTFSNYYIHWVRQAP | NCKSSQSVFYSSNNKNQLAW |
| | | GQGLEWMGWLNPNSGNTGY | YQQKPGQPPKLLIYWASTRES |
| | | AQRFQGRVTMTRDTSTSTVY | GVPDRFSGSGSGTDFTLTISSL |
| | | MELSSLRSEDTAVYYCARDL | QAEDVAVYYCQQYYSIPLTF |
| | | MTTVVTPGDYGMDVWGQGT | GQGTKLEIK (SEQ ID NO: 317) |
| | | TVTVSS (SEQ ID NO: 325) | |
| G7 | G7(2C02) | QVQLVQSGAEVKKPGASMKV | DIQMTQSPSSLSASVGDRVTIT |
| | | SCKASGYTFTTDGISWVRQAP | CQASQDIFKYLNWYQQKPGK |
| | | GQGLEWMGRIYPHSGYTEYA | APKLLIYAASTLQSGVPSRFS |
| | | KKFKGRVTMTRDTSTSTVYM | GSGSGTDFTLTISSLQPEDFAT |
| | | ELSSLRSEDTAVYYCARQDGG | YYCQQSYSTPPTFGQGTRLEI |
| | | AFAFDIWGQGTMVTVSS (SEQ | K (SEQ ID NO: 318) |
| | | ID NO: 326) | |
| G7 | G7(1A03) | QVQLVQSGAEVKKPGASVKV | DIQMTQSPSSLSASVGDRVTIT |
| | | SCKASGYTFTSQYMHWVRQA | CRASQSISTWLAWYQQKPGK |
| | | PGQGLEWMGWISPNNGDTNY | APKLLIYYASSLQSGVPSRFSG |
| | | AQKFQGRVTMTRDTSTSTVY | SGSGTDFTLTISSLQPEDFATY |
| | | MELSSLRSEDTAVYYCARELG | YCQQSYSFPYTFGQGTKVEIK |
| | | YYYGMDVWGQGTTVTVSS | (SEQ ID NO: 319) |
| | | (SEQ ID NO: 327) | |
| G7 | G7(2E09) | QVQLVQSGAEVKKPGSSVKV | DIVMTQSPLSLPVTPGEPASIS |
| | | SCKASRYTFTSYDINWVRQAP | CSSSQSLLHSNGYNYLDWYL |
| | | GQGLEWMGRIIPMLNIANYAP | QKPGQSPQLLIYLGSNRASGV |
| | | KFQGRVTITADESTSTAYMEL | PDRFSGSGSGTDFTLKISRVEA |
| | | SSLRSEDTAVYYCARALIFGV | EDVGVYYCMQALQTPLTFGG |
| | | PLLPYGMDVWGQGTTVTVSS | GTKVEIK (SEQ ID NO: 320) |
| | | (SEQ ID NO: 328) | |
| G7 | G7(1F08) | EVQLLQSGGGLVQPGGSLRLS | DIQMTQSPSSLSASVGDRVTIT |
| | | CAASGFTFSSSWMHWVRQAP | CQASQDISNYLNWYQQKPGK |
| | | GKGLEWVSFISTSSGYIYYADS | APKLLIYSASNLRSGVPSRFSG |
| | | VKGRFTISRDNSKNTLYLQMN | SGSGTDFTLTISSLQPEDFATY |
| | | SLRAEDTAVYYCAKDLATVG | YCQQGNTFPLTFGQGTKVEIK |
| | | EPYYYYGMDVWGQGTTVTV | (SEQ ID NO: 321) |
| | | SS (SEQ ID NO: 329) | |
| G7 | G7(3A09) | QVQLVQSGAEVKKPGSSVKV | DIVMTQSPLSLPVTPGEPASIS |
| | | SCKASGDTFNTYALSWVRQA | CRSSQSLLHSNGYNYLDWYL |
| | | PGQGLEWMGWMNPNSGNAG | QKPGQSPQLLIYLGSNRASGV |
| | | YAQKFQGRVTITADESTSTAY | PDRFSGSGSGTDFTLKISRVEA |
| | | MELSSLRSEDTAVYYCARLW | EDVGVYYCMQGSHWPPSFG |
| | | FGELHYYYYYGMDVWGQGT | QGTRLEIK (SEQ ID NO: 322) |
| | | MVTVSS (SEQ ID NO: 330) | |

TABLE 30
CDR sequences for G7 selective binders selective for HLA-PEPTIDE
Target HLA-A*02:01_LLASSILCA (SEQ ID NO: 8)

| Target | Clone | | | | | | |
|---|---|---|---|---|---|---|---|
| group | name | CDR-H1 | CDR-H2 | CDR-H3 | CDR-L1 | CDR-L2 | CDR-L3 |
| G7 | G7(1C06) | GTFSNY | GIINPG | CARDG | RASQSV | GASTRAT | CHHY |
| | | GIS (SEQ | GSTSYA | YDFWS | SSSNLA | (SEQ ID | GRSHT |
| | | ID NO: | (SEQ ID | GYTSDD | (SEQ ID | NO: 546) | F (SEQ |
| | | 658) | NO: 659) | YW | NO: 660) | | ID NO: |
| | | | | (SEQ ID | | | 308) |
| | | | | NO: 300) | | | |
| G7 | G7(1G10) | FTFSSY | SGISGS | CASDY | RASQDI | AASSLQS | CQQA |
| | | AMH | GGSTYY | GDYR | RNDLG | (SEQ ID | NAFPP |
| | | (SEQ ID | A (SEQ | (SEQ ID | (SEQ ID | NO: 466) | TF |
| | | NO: 661) | ID NO: | NO: 301) | NO: 663) | | (SEQ |
| | | | 662) | | | | ID NO: |
| | | | | | | | 309) |
| G7 | G7(1B04) | YTFSNY | GWLNP | CARDL | KSSQSV | WASTRES | CQQY |
| | | YIH | NSGNTG | MTTVV | FYSSNN | (SEQ ID | YSIPLT |
| | | (SEQ ID | YA (SEQ | TPGDYG | KNQLA | NO: 667) | F (SEQ |
| | | NO: 664 | ID NO: | MDVW | (SEQ ID | | ID NO: |
| | | | 665) | (SEQ ID | NO: 666) | | 310) |
| | | | | NO: 302) | | | |
| G7 | G7(2C02) | YTFTTD | GRIYPH | CARQD | QASQDI | AASTLQS | CQQSY |
| | | GIS (SEQ | SGYTEY | GGAFAF | FKYLN | (SEQ ID | STPPTF |
| | | ID NO: | A (SEQ | DIW | (SEQ ID | NO: 474) | (SEQ |
| | | 668) | ID NO: | (SEQ ID | NO: 670) | | ID NO: |
| | | | 669) | NO: 303) | | | 311) |
| G7 | G7(1A03) | YTFTSQ | GWISPN | CARELG | RASQSI | YASSLQS | CQQSY |
| | | YMH | NGDTN | YYYGM | STWLA | (SEQ ID | SFPYT |
| | | (SEQ ID | YA (SEQ | DVW | (SEQ ID | NO: 478) | F (SEQ |
| | | NO: 655) | ID NO: | (SEQ ID | NO: 591) | | ID NO: |
| | | | 671) | NO: 304) | | | 312) |
| G7 | G7(2E09) | YTFTSY | GRIIPM | CARALI | SSSQSL | LGSNRAS | CMQA |
| | | DIN | LNIANY | FGVPLL | LHSNGY | (SEQ ID | LQTPL |
| | | (SEQ ID | A (SEQ | PYGMD | NYLD | NO: 505) | TF |
| | | NO: 436) | ID NO: | VW | (SEQ ID | | (SEQ |
| | | | 672) | (SEQ ID | NO: 673) | | ID NO: |
| | | | | NO: 305) | | | 93) |
| G7 | G7(1F08) | FTFSSS | SFISTSS | CAKDL | QASQDI | SASNLRS | CQQG |
| | | WMH | GYIYYA | ATVGEP | SNYLN | (SEQ ID | NTFPL |
| | | (SEQ ID | (SEQ ID | YYYYG | (SEQ ID | NO: 676) | TF |
| | | NO: 674) | NO: 675) | MDVW | NO: 465) | | (SEQ |
| | | | | (SEQ ID | | | ID NO: |
| | | | | NO: 306) | | | 313) |
| G7 | G7(3A09) | DTFNTY | GWMNP | CARLW | RSSQSL | LGSNRAS | CMQG |
| | | ALS | NSGNA | FGELHY | LHSNGY | (SEQ ID | SHWPP |
| | | (SEQ ID | GYA | YYYYG | NYLD | NO: 505) | SF |
| | | NO: 677) | (SEQ ID | MDVW | (SEQ ID | | (SEQ |
| | | | NO: 678) | (SEQ ID | NO: 438) | | ID NO: |
| | | | | NO: 307) | | | 314) |

TABLE 33
Exemplary Bispecific Format 1 Constructs

| | Anti-HLA-peptide | | |
|---|---|---|---|
| Name | scFv | Linker | Anti-CD3 scFv |
| 1-G2(1H11)-hOKT3 | DIQMTQSPSSLSAS | GGGGS (SEQ ID NO: | QVQLVQSGAEVKK |
| | VGDRVTITCQASQD | 730) | PGASVKVSCKASG |
| | ISNYLNWYQQKPG | | YTFTRYTMHWVRQ |
| | KAPKLLIYAASSLQ | | APGQGLEWMGYIN |
| | SGVPSRFSGSGSGT | | PSRGYTNYNQKFK |
| | DFTLTISSLQPEDFA | | DRVTLTTDKSSSTA |
| | TYYCQQYYSYPFTF | | YMELSSLRSEDTAV |
| | GPGTKVDIKGGGG | | YYCARYYDDHYSL |
| | SGGGGSGGGGSGG | | DYWGQGTLVTVSS |
| | GGSQVQLVQSGAE | | VEGGSGGSGGSGG |
| | VKKPGASVKVSCK | | SGGVDDIQMTQSPS |
| | ASGYTFTNYYMHW | | SLSASVGDRVTITC |
| | VRQAPGQGLEWM | | SASSSVSYMNWYQ |
| | GMINPSGGGTSYA | | QKPGKAPKRLIYDT |
| | QKFQGRVTMTRDT | | SKLASGVPSRFSGS |
| | STSTVYMELSSLRS | | GSGTDFTLTISSLQP |
| | EDTAVYYCARGNP | | EDFATYYCQQWSS |
| | WELRLDYWGQGTL | | NPFTFGQGTKLEIK |
| | VTVSS (SEQ ID NO: | | (SEQ ID NO: 707) |
| | 706) | | |
| 1-G2(1H11)-anti-CD3 | DIQMTQSPSSLSAS | GGGGS (SEQ ID NO: | EVQLVESGGGLVQ |
| | VGDRVTITCQASQD | 730) | PGGSLRLSCAASGF |
| | ISNYLNWYQQKPG | | TFSTYAMNWVRQA |
| | KAPKLLIYAASSLQ | | PGKGLEWVGRIRS |
| | SGVPSRFSGSGSGT | | KYNNYATYYADSV |
| | DFTLTISSLQPEDFA | | KGRFTISRDDSKNT |
| | TYYCQQYYSYPFTF | | LYLQMNSLRAEDT |
| | GPGTKVDIKGGGG | | AVYYCVRHGNFGD |
| | SGGGGSGGGGSGG | | SYVSWFAYWGQGT |
| | GGSQVQLVQSGAE | | LVTVSSGKPGSGKP |
| | VKKPGASVKVSCK | | GSGKPGSGKPGSQ |
| | ASGYTFTNYYMHW | | AVVTQEPSLTVSPG |
| | VRQAPGQGLEWM | | GTVTLTCGSSTGAV |
| | GMINPSGGGTSYA | | TTSNYANWVQQKP |
| | QKFQGRVTMTRDT | | GKSPRGLIGGTNKR |
| | STSTVYMELSSLRS | | APGVPARFSGSLLG |
| | EDTAVYYCARGNP | | GKAALTISGAQPED |
| | WELRLDYWGQGTL | | EADYYCALWYSNH |
| | VTVSS (SEQ ID NO: | | WVFGGGTKLTVL |
| | 706) | | (SEQ ID NO: 708) |
| 1-G5(1C12)-hOKT3 | DIQMTQSPSSLSAS | GGGGS (SEQ ID NO: | QVQLVQSGAEVKK |
| | VGDRVTITCRASQS | 730) | PGASVKVSCKASG |
| | ISSWLAWYQQKPG | | YTFTRYTMHWVRQ |
| | KAPKLLIYAASTLQ | | APGQGLEWMGYIN |
| | SGVPSRFSGSGSGT | | PSRGYTNYNQKFK |
| | DFTLTISSLQPEDFA | | DRVTLTTDKSSSTA |
| | TYYCQQSYSIPLTF | | YMELSSLRSEDTAV |
| | GGGTKVEIKGGGG | | YYCARYYDDHYSL |
| | SGGGGSGGGGSGG | | DYWGQGTLVTVSS |
| | GGSQVQLVQSGAE | | VEGGSGGSGGSGG |
| | VKKPGSSVKVSCK | | SGGVDDIQMTQSPS |
| | ASGGTFSNFGVSW | | SLSASVGDRVTITC |
| | LRQAPGQGLEWMG | | SASSSVSYMNWYQ |
| | GIIPILGTANYAQKF | | QKPGKAPKRLIYDT |
| | QGRVTITADESTST | | SKLASGVPSRFSGS |
| | AYMELSSLRSEDTA | | GSGTDFTLTISSLQP |
| | VYYCATPTNSGYY | | EDFATYYCQQWSS |
| | GPYYYYGMDVWG | | NPFTFGQGTKLEIK |
| | QGTTVTVSS (SEQ | | (SEQ ID NO: 707) |
| | ID NO: 709) | | |
| 1-G5(1C12)-anti-CD3 | DIQMTQSPSSLSAS | GGGGS (SEQ ID NO: | EVQLVESGGGLVQ |
| | VGDRVTITCRASQS | 730) | PGGSLRLSCAASGF |
| | ISSWLAWYQQKPG | | TFSTYAMNWVRQA |
| | KAPKLLIYAASTLQ | | PGKGLEWVGRIRS |
| | SGVPSRFSGSGSGT | | KYNNYATYYADSV |
| | DFTLTISSLQPEDFA | | KGRFTISRDDSKNT |
| | TYYCQQSYSIPLTF | | LYLQMNSLRAEDT |
| | GGGTKVEIKGGGG | | AVYYCVRHGNFGD |
| | SGGGGSGGGGSGG | | SYVSWFAYWGQGT |
| | GGSQVQLVQSGAE | | LVTVSSGKPGSGKP |
| | VKKPGSSVKVSCK | | GSGKPGSGKPGSQ |
| | ASGGTFSNFGVSW | | AVVTQEPSLTVSPG |
| | LRQAPGQGLEWMG | | GTVTLTCGSSTGAV |
| | GIIPILGTANYAQKF | | TTSNYANWVQQKP |
| | QGRVTITADESTST | | GKSPRGLIGGTNKR |
| | AYMELSSLRSEDTA | | APGVPARFSGSLLG |
| | VYYCATPTNSGYY | | GKAALTISGAQPED |
| | GPYYYYGMDVWG | | EADYYCALWYSNH |
| | QGTTVTVSS (SEQ | | WVFGGGTKLTVL |
| | ID NO: 709) | | (SEQ ID NO: 708) |

TABLE 34
Exemplary Bispecific Format 2 Constructs

| | | CH1_CH2 | Linker | | |
|---|---|---|---|---|---|
| | | CH3 | (Chains | scFv | LC |
| | VH | (Chains | 1 | (Chains | (Chains |
| | (Chains | 1 and | and | 1 and | 3 and |
| Name | 1 and 2 | 2) | 2) | 2) | 4) |
| 2- | QVQLVQS | ASTKGPS | GGGGSGG | QVQLVQS | DIQMTQS |
| hOKT3- | GAEVKKP | VFPLAPS | GGS | GAEVKKP | PSSLSAS |
| G2 | GASVKVS | SKSTSGG | (SEQ ID | GASVKVS | VGDRVTI |
| (1H11) | CKASGYT | TAALGCL | NO: | CKASGYT | TCSASSS |
| | FTRYTMH | VKDYFPE | 107) | FTNYYMH | VSYMNWY |
| | WVRQAPG | PVTVSWN | | WVRQAPG | QQKPGKA |
| | QGLEWMG | SGALTSG | | QGLEWMG | PKRLIYD |
| | YINPSRG | VHTFPAV | | MINPSGG | TSKLASG |
| | YTNYNQK | LQSSGLY | | GTSYAQK | VPSRFSG |
| | FKDRVTL | SLSSVVT | | FQGRVTM | SGSGTDF |
| | TTDKSSS | VPSSSLG | | TRDTSTS | TLTISSL |
| | TAYMELS | TQTYICN | | TVYMELS | OPEDFAT |
| | SLRSEDT | VNHKPSN | | SLRSEDT | YYCQQWS |
| | AVYYCAR | TKVDKRV | | AVYYCAR | SNPFTFG |
| | YYDDHYS | EPKSCDK | | GNPWELR | QGTKLEI |
| | LDYWGQG | THTCPPC | | LDYWGQG | KRTVAAP |
| | TLVTVSS | PAPELLG | | TLVTVSS | SVFIFPP |
| | (SEQ ID | GPSVFLF | | GGGGSGG | SDEQLKS |
| | NO: | PPKPKDT | | GGSGGGG | GTASVVC |
| | 126) | LMISRTP | | SGGGGSD | LLNNFYP |
| | | EVTCVVV | | IQMTQSP | REAKVQW |
| | | DVSHEDP | | SSLSASV | KVDNALQ |
| | | EVKFNWY | | GDRVTIT | SGNSQES |
| | | VDGVEVH | | CQASQDI | VTEQDSK |
| | | NAKTKPR | | SNYLNWY | DSTYSLS |
| | | EEQYQST | | QQKPGKA | STLTLSK |
| | | YRVVSVL | | PKLLIYA | ADYEKHK |
| | | TVLHQDW | | ASSLQSG | VYACEVT |
| | | LNGKEYK | | VPSRFSG | HQGLSSP |
| | | CKVSNKA | | SGSGTDF | VTKSFNR |
| | | LPAPIEK | | TLTISSL | GEC |
| | | TISKAKG | | QPEDFAT | (SEQ ID |
| | | QPREPQV | | YYCQQYY | NO: |
| | | YTLPPSR | | SYPFTFG | 712) |
| | | EEMTKNQ | | PGTKVDI | |
| | | VSLTCLV | | K | |
| | | KGFYPSD | | (SEQ ID | |
| | | IAVEWES | | NO: | |
| | | NGQPENN | | 711) | |
| | | YKTTPPV | | | |
| | | LDSDGSF | | | |
| | | FLYSKLT | | | |
| | | VDKSRWQ | | | |
| | | QGNVFSC | | | |
| | | SVMHEAL | | | |
| | | HNHYTQK | | | |
| | | SLSLSPG | | | |
| | | K | | | |
| | | (SEQ ID | | | |
| | | NO: | | | |
| | | 710) | | | |
| 2-anti- | EVQLVES | ASTKGPS | GGGGSGG | QVQLVQS | QAVVTQE |
| CD3- | GGGLVQP | VFPLAPS | GGS | GAEVKKP | PSLTVSP |
| G2 | GGSLRLS | SKSTSGG | (SEQ ID | GASVKVS | GGTVTLT |
| (1H11) | CAASGFT | TAALGCL | NO: | CKASGYT | CGSSTGA |
| | FSTYAMN | VKDYFPE | 107) | FTNYYMH | VTTSNYA |
| | WVRQAPG | PVTVSWN | | WVRQAPG | NWVQQKP |
| | KGLEWVG | SGALTSG | | QGLEWMG | GKSPRGL |
| | RIRSKYN | VHTFPAV | | MINPSGG | IGGTNKR |
| | NYATYYA | LQSSGLY | | GTSYAQK | APGVPAR |
| | DSVKGRF | SLSSVVT | | FQGRVTM | FSGSLLG |
| | TISRDDS | VPSSSLG | | TRDTSTS | GKAALTI |
| | KNTLYLQ | TQTYICN | | TVYMELS | SGAQPED |
| | MNSLRAE | VNHKPSN | | SLRSEDT | EADYYCA |
| | DTAVYYC | TKVDKRV | | AVYYCAR | LWYSNHW |
| | VRHGNFG | EPKSCDK | | GNPWELR | VFGGGTK |
| | DSYVSWF | THTCPPC | | LDYWGQG | LTVLRTV |
| | AYWGQGT | PAPELLG | | TLVTVSS | AAPSVFI |
| | LVTVSS | GPSVFLF | | GGGGSGG | FPPSDEQ |
| | (SEQ ID | PPKPKDT | | GGSGGGG | LKSGTAS |
| | NO: | LMISRTP | | SGGGGSD | VVCLLNN |
| | 132) | EVTCVVV | | IQMTQSP | FYPREAK |
| | | DVSHEDP | | SSLSASV | VQWKVDN |
| | | EVKFNWY | | GDRVTIT | ALQSGNS |
| | | VDGVEVH | | CQASQDI | QESVTEQ |
| | | NAKTKPR | | SNYLNWY | DSKDSTY |
| | | EEQYQST | | QQKPGKA | SLSSTLT |
| | | YRVVSVL | | PKLLIYA | LSKADYE |
| | | TVLHQDW | | ASSLQSG | KHKVYAC |
| | | LNGKEYK | | VPSRFSG | EVTHQGL |
| | | CKVSNKA | | SGSGTDF | SSPVTKS |
| | | LPAPIEK | | TLTISSL | FNRGEC |
| | | TISKAKG | | QPEDFAT | (SEQ ID |
| | | QPREPQV | | YYCQQYY | NO: |
| | | YTLPPSR | | SYPFTFG | 713) |
| | | EEMTKNQ | | PGTKVDI | |
| | | VSLTCLV | | K | |
| | | KGFYPSD | | (SEQ ID | |
| | | IAVEWES | | NO: | |
| | | NGQPENN | | 711) | |
| | | YKTTPPV | | | |
| | | LDSDGSF | | | |
| | | FLYSKLT | | | |
| | | VDKSRWQ | | | |
| | | QGNVFSC | | | |
| | | SVMHEAL | | | |
| | | HNHYTQK | | | |
| | | SLSLSPG | | | |
| | | K | | | |
| | | (SEQ ID | | | |
| | | NO: 710) | | | |
| 2- | QVQLVQS | ASTKGPS | GGGGSGG | QVQLVQS | DIQMTQS |
| hOKT3- | GAEVKKP | VFPLAPS | GGS | GAEVKKP | PSSLSAS |
| G5 | GASVKVS | SKSTSGG | (SEQ ID | GSSVKVS | VGDRVTI |
| (1C12) | CKASGYT | TAALGCL | NO: | CKASGGT | TCSASSS |
| | FTRYTMH | VKDYFPE | 107) | FSNFGVS | VSYMNWY |
| | WVRQAPG | PVTVSWN | | WLRQAPG | QQKPGKA |
| | QGLEWMG | SGALTSG | | QGLEWMG | PKRLIYD |
| | YINPSRG | VHTFPAV | | GIIPILG | TSKLASG |
| | YTNYNQK | LQSSGLY | | TANYAQK | VPSRFSG |
| | FKDRVTL | SLSSVVT | | FQGRVTI | SGSGTDF |
| | TTDKSSS | VPSSSLG | | TADESTS | TLTISSL |
| | TAYMELS | TQTYICN | | TAYMELS | QPEDFAT |
| | SLRSEDT | VNHKPSN | | SLRSEDT | YYCQQWS |
| | AVYYCAR | TKVDKRV | | AVYYCAT | SNPFTFG |
| | YYDDHYS | EPKSCDK | | PTNSGYY | QGTKLEI |
| | LDYWGQG | THTCPPC | | GPYYYYG | KRTVAAP |
| | TLVTVSS | PAPELLG | | MDVWGQG | SVFIFPP |
| | (SEQ ID | GPSVFLF | | TTVTVSS | SDEQLKS |
| | NO: | PPKPKDT | | GGGGSGG | GTASVVC |
| | 126) | LMISRTP | | GGSGGGG | LLNNFYP |
| | | EVTCVVV | | SGGGGSD | REAKVQW |
| | | DVSHEDP | | IQMTQSP | KVDNALQ |
| | | EVKFNWY | | SSLSASV | SGNSQES |
| | | VDGVEVH | | GDRVTIT | VTEQDSK |
| | | NAKTKPR | | CRASQSI | DSTYSLS |
| | | EEQYQST | | SSWLAWY | STLTLSK |
| | | YRVVSVL | | QQKPGKA | ADYEKHK |
| | | TVLHQDW | | PKLLIYA | VYACEVT |
| | | LNGKEYK | | ASTLQSG | HQGLSSP |
| | | CKVSNKA | | VPSRFSG | VTKSFNR |
| | | LPAPIEK | | SGSGTDF | GEC |
| | | TISKAKG | | TLTISSL | (SEQ ID |
| | | QPREPQV | | QPEDFAT | NO: |
| | | YTLPPSR | | YYCQQSY | 712) |
| | | EEMTKNQ | | SIPLTFG | |
| | | VSLTCLV | | GGTKVEI | |
| | | KGFYPSD | | K | |
| | | IAVEWES | | (SEQ ID | |
| | | NGQPENN | | NO: | |
| | | YKTTPPV | | 714) | |
| | | LDSDGSF | | | |
| | | FLYSKLT | | | |
| | | VDKSRWQ | | | |
| | | QGNVFSC | | | |
| | | SVMHEAL | | | |
| | | HNHYTQK | | | |
| | | SLSLSPG | | | |
| | | K | | | |
| | | (SEQ ID | | | |
| | | NO: | | | |
| | | 710) | | | |
| 2- | EVQLVES | ASTKGPS | GGGGSGG | QVQLVQS | QAVVTQE |
| anti- | GGGLVQP | VFPLAPS | GGS | GAEVKKP | PSLTVSP |
| CD3- | GGSLRLS | SKSTSGG | (SEQ ID | GSSVKVS | GGTVTLT |
| G5 | CAASGFT | TAALGCL | NO: | CKASGGT | CGSSTGA |
| (1C12) | FSTYAMN | VKDYFPE | 107) | FSNFGVS | VTTSNYA |
| | WVRQAPG | PVTVSWN | | WLRQAPG | NWVQQKP |
| | KGLEWVG | SGALTSG | | QGLEWMG | GKSPRGL |
| | RIRSKYN | VHTFPAV | | GIIPILG | IGGTNKR |
| | NYATYYA | LQSSGLY | | TANYAQK | APGVPAR |
| | DSVKGRF | SLSSVVT | | FQGRVTI | FSGSLLG |
| | TISRDDS | VPSSSLG | | TADESTS | GKAALTI |
| | KNTLYLQ | TQTYICN | | TAYMELS | SGAQPED |
| | MNSLRAE | VNHKPSN | | SLRSEDT | EADYYCA |
| | DTAVYYC | TKVDKRV | | AVYYCAT | LWYSNHW |
| | VRHGNFG | EPKSCDK | | PTNSGYY | VFGGGTK |
| | DSYVSWF | THTCPPC | | GPYYYYG | LTVLRTV |
| | AYWGQGT | PAPELLG | | MDVWGQG | AAPSVFI |
| | LVTVSS | GPSVFLF | | TTVTVSS | FPPSDEQ |
| | (SEQ ID | PPKPKDT | | GGGGSGG | LKSGTAS |
| | NO: 132) | LMISRTP | | GGSGGGG | VVCLLNN |
| | | EVTCVVV | | SGGGGSD | FYPREAK |
| | | DVSHEDP | | IQMTQSP | VQWKVDN |
| | | EVKFNWY | | SSLSASV | ALQSGNS |
| | | VDGVEVH | | GDRVTIT | QESVTEQ |
| | | NAKTKPR | | CRASQSI | DSKDSTY |
| | | EEQYQST | | SSWLAWY | SLSSTLT |
| | | YRVVSVL | | QQKPGKA | LSKADYE |
| | | TVLHQDW | | PKLLIYA | KHKVYAC |
| | | LNGKEYK | | ASTLQSG | EVTHQGL |
| | | CKVSNKA | | VPSRFSG | SSPVTKS |
| | | LPAPIEK | | SGSGTDF | FNRGEC |
| | | TISKAKG | | TLTISSL | (SEQ ID |
| | | QPREPQV | | QPEDFAT | NO: |
| | | YTLPPSR | | YYCQQSY | 713) |
| | | EEMTKNQ | | SIPLTFG | |
| | | VSLTCLV | | GGTKVEI | |
| | | KGFYPSD | | K | |
| | | IAVEWES | | (SEQ ID | |
| | | NGQPENN | | NO: | |
| | | YKTTPPV | | 714) | |
| | | LDSDGSF | | | |
| | | FLYSKLT | | | |
| | | VDKSRWQ | | | |
| | | QGNVFSC | | | |
| | | SVMHEAL | | | |
| | | HNHYTQK | | | |
| | | SLSLSPG | | | |
| | | K | | | |
| | | (SEQ ID | | | |
| | | NO: | | | |
| | | 710) | | | |

TABLE 35
Exemplary Bispecific Format 3 Constructs

| | | CH1_ | | | |
|---|---|---|---|---|---|
| | | CH2_ | | Linker- | |
| | VH | CH3 | scFv | Fc | LC |
| | (Chain | (Chain | (Chain | (Chain | (Chain |
| Name | 2) | 2) | 1) | 1) | 3) |
| 3- | QVQLVQS | ASTKGPS | QVQLVQS | GGGGSEP | DIQMTQS |
| hOKT3- | GAEVKKP | VFPLAPS | GAEVKKP | KSSDKTH | PSSLSAS |
| G2 | GASVKVS | SKSTSGG | GASVKVS | TCPPCPA | VGDRVTI |
| (1H11) | CKASGYT | TAALGCL | CKASGYT | PELLGGP | TCSASSS |
| | FTRYTMH | VKDYFPE | FTNYYMH | SVFLFPP | VSYMNWY |
| | WVRQAPG | PVTVSWN | WVRQAPG | KPKDTLM | QQKPGKA |
| | QGLEWMG | SGALTSG | QGLEWMG | ISRTPEV | PKRLIYD |
| | YINPSRG | VHTFPAV | MINPSGG | TCVVVDV | TSKLASG |
| | YTNYNQK | LQSSGLY | GTSYAQK | SHEDPEV | VPSRFSG |
| | FKDRVTL | SLSSVVT | FQGRVTM | KFNWYVD | SGSGTDF |
| | TTDKSSS | VPSSSLG | TRDTSTS | GVEVHNA | TLTISSL |
| | TAYMELS | TQTYICN | TVYMELS | KTKPREE | QPEDFAT |
| | SLRSEDT | VNHKPSN | SLRSEDT | QYQSTYR | YYCQQWS |
| | AVYYCAR | TKVDKRV | AVYYCAR | VVSVLTV | SNPFTFG |
| | YYDDHYS | EPKSCDK | GNPWELR | LHQDWLN | QGTKLEI |
| | LDYWGQG | THTCPPC | LDYWGQG | GKEYKCK | KRTVAAP |
| | TLVTVSS | PAPELLG | TLVTVSS | VSNKALP | SVFIFPP |
| | (SEQ ID | GPSVFLF | GGGGSGG | APIEKTI | SDEQLKS |
| | NO: | PPKPKDT | GGSGGGG | SKAKGQP | GTASVVC |
| | 126) | LMISRTP | SGGGGSD | REPQVYT | LLNNFYP |
| | | EVTCVVV | IQMTQSP | LPPCREE | REAKVQW |
| | | DVSHEDP | SSLSASV | MTKNQVS | KVDNALQ |
| | | EVKFNWY | GDRVTIT | LWCLVKG | SGNSQES |
| | | VDGVEVH | CQASQDI | FYPSDIA | VTEQDSK |
| | | NAKTKPR | SNYLNWY | VEWESNG | DSTYSLS |
| | | EEQYQST | QQKPGKA | QPENNYK | STLTLSK |
| | | YRVVSVL | PKLLIYA | TTPPVLD | ADYEKHK |
| | | TVLHQDW | ASSLQSG | SDGSFFL | VYACEVT |
| | | LNGKEYK | VPSRFSG | YSKLTVD | HQGLSSP |
| | | CKVSNKA | SGSGTDF | KSRWQQG | VTKSFNR |
| | | LPAPIEK | TLTISSL | NVFSCSV | GEC |
| | | TISKAKG | QPEDFAT | MHEALHN | (SEQ ID |
| | | QPREPQV | YYCQQYY | HYTQKSL | NO: |
| | | CTLPPSR | SYPFTFG | SLSPGK | 712) |
| | | EEMTKNQ | PGTKVDI | (SEQ ID | |
| | | VSLSCAV | K | NO: | |
| | | KGFYPSD | (SEQ ID | 716) | |
| | | IAVEWES | NO: | | |
| | | NGQPENN | 711) | | |
| | | YKTTPPV | | | |
| | | LDSDGSF | | | |
| | | FLVSKLT | | | |
| | | VDKSRWQ | | | |
| | | QGNVFSC | | | |
| | | SVMHEAL | | | |
| | | HNRFTQK | | | |
| | | SLSLSPG | | | |
| | | K | | | |
| | | (SEQ ID | | | |
| | | NO: | | | |
| | | 715) | | | |
| 3- | EVQLVES | ASTKGPS | QVQLVQS | GGGGSEP | QAVVTQE |
| anti- | GGGLVQP | VFPLAPS | GAEVKKP | KSSDKTH | PSLTVSP |
| CD3- | GGSLRLS | SKSTSGG | GASVKVS | TCPPCPA | GGTVTLT |
| G2 | CAASGFT | TAALGCL | CKASGYT | PELLGGP | CGSSTGA |
| (1H11) | FSTYAMN | VKDYFPE | FTNYYMH | SVFLFPP | VTTSNYA |
| | WVRQAPG | PVTVSWN | WVRQAPG | KPKDTLM | NWVQQKP |
| | KGLEWVG | SGALTSG | QGLEWMG | ISRTPEV | GKSPRGL |
| | RIRSKYN | VHTFPAV | MINPSGG | TCVVVDV | IGGTNKR |
| | NYATYYA | LQSSGLY | GTSYAQK | SHEDPEV | APGVPAR |
| | DSVKGRF | SLSSVVT | FQGRVTM | KFNWYVD | FSGSLLG |
| | TISRDDS | VPSSSLG | TRDTSTS | GVEVHNA | GKAALTI |
| | KNTLYLQ | TQTYICN | TVYMELS | KTKPREE | SGAQPED |
| | MNSLRAE | VNHKPSN | SLRSEDT | QYQSTYR | EADYYCA |
| | DTAVYYC | TKVDKRV | AVYYCAR | VVSVLTV | LWYSNHW |
| | VRHGNFG | EPKSCDK | GNPWELR | LHQDWLN | VFGGGTK |
| | DSYVSWF | THTCPPC | LDYWGQG | GKEYKCK | LTVLRTV |
| | AYWGQGT | PAPELLG | TLVTVSS | VSNKALP | AAPSVFI |
| | LVTVSS | GPSVFLF | GGGGSGG | APIEKTI | FPPSDEQ |
| | (SEQ ID | PPKPKDT | GGSGGGG | SKAKGQP | LKSGTAS |
| | NO: | LMISRTP | SGGGGSD | REPQVYT | VVCLLNN |
| | 132) | EVTCVVV | IQMTQSP | LPPCREE | FYPREAK |
| | | DVSHEDP | SSLSASV | MTKNQVS | VQWKVDN |
| | | EVKFNWY | GDRVTIT | LWCLVKG | ALQSGNS |
| | | VDGVEVH | CQASQDI | FYPSDIA | QESVTEQ |
| | | NAKTKPR | SNYLNWY | VEWESNG | DSKDSTY |
| | | EEQYQST | QQKPGKA | QPENNYK | SLSSTLT |
| | | YRVVSVL | PKLLIYA | TTPPVLD | LSKADYE |
| | | TVLHQDW | ASSLQSG | SDGSFFL | KHKVYAC |
| | | LNGKEYK | VPSRFSG | YSKLTVD | EVTHQGL |
| | | CKVSNKA | SGSGTDF | KSRWQQG | SSPVTKS |
| | | LPAPIEK | TLTISSL | NVFSCSV | FNRGEC |
| | | TISKAKG | QPEDFAT | MHEALHN | (SEQ ID |
| | | QPREPQV | YYCQQYY | HYTQKSL | NO: |
| | | CTLPPSR | SYPFTFG | SLSPGK | 713) |
| | | EEMTKNQ | PGTKVDI | (SEQ ID | |
| | | VSLSCAV | K | NO: | |
| | | KGFYPSD | (SEQ ID | 716) | |
| | | IAVEWES | NO: | | |
| | | NGQPENN | 711) | | |
| | | YKTTPPV | | | |
| | | LDSDGSF | | | |
| | | FLVSKLT | | | |
| | | VDKSRWQ | | | |
| | | QGNVFSC | | | |
| | | SVMHEAL | | | |
| | | HNRFTQK | | | |
| | | SLSLSPG | | | |
| | | K | | | |
| | | (SEQ ID | | | |
| | | NO: | | | |
| | | 715) | | | |
| 3- | QVQLVQS | ASTKGPS | QVQLVQS | GGGGSEP | DIQMTQS |
| hOKT3- | GAEVKKP | VFPLAPS | GAEVKKP | KSSDKTH | PSSLSAS |
| G5 | GASVKVS | SKSTSGG | GSSVKVS | TCPPCPA | VGDRVTI |
| (1C12) | CKASGYT | TAALGCL | CKASGGT | PELLGGP | TCSASSS |
| | FTRYTMH | VKDYFPE | FSNFGVS | SVFLFPP | VSYMNWY |
| | WVRQAPG | PVTVSWN | WLRQAPG | KPKDTLM | QQKPGKA |
| | QGLEWMG | SGALTSG | QGLEWMG | ISRTPEV | PKRLIYD |
| | YINPSRG | VHTFPAV | GIIPILG | TCVVVDV | TSKLASG |
| | YTNYNQK | LQSSGLY | TANYAQK | SHEDPEV | VPSRFSG |
| | FKDRVTL | SLSSVVT | FQGRVTI | KFNWYVD | SGSGTDF |
| | TTDKSSS | VPSSSLG | TADESTS | GVEVHNA | TLTISSL |
| | TAYMELS | TQTYICN | TAYMELS | KTKPREE | QPEDFAT |
| | SLRSEDT | VNHKPSN | SLRSEDT | QYQSTYR | YYCQQWS |
| | AVYYCAR | TKVDKRV | AVYYCAT | VVSVLTV | SNPFTFG |
| | YYDDHYS | EPKSCDK | PTNSGYY | LHQDWLN | QGTKLEI |
| | LDYWGQG | THTCPPC | GPYYYYG | GKEYKCK | KRTVAAP |
| | TLVTVSS | PAPELLG | MDVWGQG | VSNKALP | SVFIFPP |
| | (SEQ ID | GPSVFLF | TTVTVSS | APIEKTI | SDEQLKS |
| | NO: | PPKPKDT | GGGGSGG | SKAKGQP | GTASVVC |
| | 126) | LMISRTP | GGSGGGG | REPQVYT | LLNNFYP |
| | | EVTCVVV | SGGGGSD | LPPCREE | REAKVQW |
| | | DVSHEDP | IQMTQSP | MTKNQVS | KVDNALQ |
| | | EVKFNWY | SSLSASV | LWCLVKG | SGNSQES |
| | | VDGVEVH | GDRVTIT | FYPSDIA | VTEQDSK |
| | | NAKTKPR | CRASQSI | VEWESNG | DSTYSLS |
| | | EEQYQST | SSWLAWY | QPENNYK | STLTLSK |
| | | YRVVSVL | QQKPGKA | TTPPVLD | ADYEKHK |
| | | TVLHQDW | PKLLIYA | SDGSFFL | VYACEVT |
| | | LNGKEYK | ASTLQSG | YSKLTVD | HQGLSSP |
| | | CKVSNKA | VPSRFSG | KSRWQQG | VTKSFNR |
| | | LPAPIEK | SGSGTDF | NVFSCSV | GEC |
| | | TISKAKG | TLTISSL | MHEALHN | (SEQ ID |
| | | QPREPQV | QPEDFAT | HYTQKSL | NO: |
| | | CTLPPSR | YYCQQSY | SLSPGK | 712) |
| | | EEMTKNQ | SIPLTFG | (SEQ ID | |
| | | VSLSCAV | GGTKVEI | NO: | |
| | | KGFYPSD | K | 716) | |
| | | IAVEWES | (SEQ ID | | |
| | | NGQPENN | NO: | | |
| | | YKTTPPV | 714) | | |
| | | LDSDGSF | | | |
| | | FLVSKLT | | | |
| | | VDKSRWQ | | | |
| | | QGNVFSC | | | |
| | | SVMHEAL | | | |
| | | HNRFTQK | | | |
| | | SLSLSPG | | | |
| | | K | | | |
| | | (SEQ ID | | | |
| | | NO: | | | |
| | | 715) | | | |
| 3- | EVQLVES | ASTKGPS | QVQLVQS | GGGGSEP | QAVVTQE |
| anti- | GGGLVQP | VFPLAPS | GAEVKKP | KSSDKTH | PSLTVSP |
| CD3- | GGSLRLS | SKSTSGG | GSSVKVS | TCPPCPA | GGTVTLT |
| G5 | CAASGFT | TAALGCL | CKASGGT | PELLGGP | CGSSTGA |
| (1C12) | FSTYAMN | VKDYFPE | FSNFGVS | SVFLFPP | VTTSNYA |
| | WVRQAPG | PVTVSWN | WLRQAPG | KPKDTLM | NWVQQKP |
| | KGLEWVG | SGALTSG | QGLEWMG | ISRTPEV | GKSPRGL |
| | RIRSKYN | VHTFPAV | GIIPILG | TCVVVDV | IGGTNKR |
| | NYATYYA | LQSSGLY | TANYAQK | SHEDPEV | APGVPAR |
| | DSVKGRF | SLSSVVT | FQGRVTI | KFNWYVD | FSGSLLG |
| | TISRDDS | VPSSSLG | TADESTS | GVEVHNA | GKAALTI |
| | KNTLYLQ | TQTYICN | TAYMELS | KTKPREE | SGAQPED |
| | MNSLRAE | VNHKPSN | SLRSEDT | QYQSTYR | EADYYCA |
| | DTAVYYC | TKVDKRV | AVYYCAT | VVSVLTV | LWYSNHW |
| | VRHGNFG | EPKSCDK | PTNSGYY | LHQDWLN | VFGGGTK |
| | DSYVSWF | THTCPPC | GPYYYYG | GKEYKCK | LTVLRTV |
| | AYWGQGT | PAPELLG | MDVWGQG | VSNKALP | AAPSVFI |
| | LVTVSS | GPSVFLF | TTVTVSS | APIEKTI | FPPSDEQ |
| | (SEQ ID | PPKPKDT | GGGGSGG | SKAKGQP | LKSGTAS |
| | NO: | LMISRTP | GGSGGGG | REPQVYT | VVCLLNN |
| | 132) | EVTCVVV | SGGGGSD | LPPCREE | FYPREAK |
| | | DVSHEDP | IQMTQSP | MTKNQVS | VQWKVDN |
| | | EVKFNWY | SSLSASV | LWCLVKG | ALQSGNS |
| | | VDGVEVH | GDRVTIT | FYPSDIA | QESVTEQ |
| | | NAKTKPR | CRASQSI | VEWESNG | DSKDSTY |
| | | EEQYQST | SSWLAWY | QPENNYK | SLSSTLT |
| | | YRVVSVL | QQKPGKA | TTPPVLD | LSKADYE |
| | | TVLHQDW | PKLLIYA | SDGSFFL | KHKVYAC |
| | | LNGKEYK | ASTLQSG | YSKLTVD | EVTHQGL |
| | | CKVSNKA | VPSRFSG | KSRWQQG | SSPVTKS |
| | | LPAPIEK | SGSGTDF | NVFSCSV | FNRGEC |
| | | TISKAKG | TLTISSL | MHEALHN | (SEQ ID |
| | | QPREPQV | QPEDFAT | HYTQKSL | NO: |
| | | CTLPPSR | YYCQQSY | SLSPGK | 713) |
| | | EEMTKNQ | SIPLTFG | (SEQ ID | |
| | | VSLSCAV | GGTKVEI | NO: | |
| | | KGFYPSD | K | 716) | |
| | | IAVEWES | (SEQ ID | | |
| | | NGQPENN | NO: | | |
| | | YKTTPPV | 714) | | |
| | | LDSDGSF | | | |
| | | FLVSKLT | | | |
| | | VDKSRWQ | | | |
| | | QGNVFSC | | | |
| | | SVMHEAL | | | |
| | | HNRFTQK | | | |
| | | SLSLSPG | | | |
| | | K | | | |
| | | (SEQ ID | | | |
| | | NO: | | | |
| | | 715) | | | |

TABLE 36
Exemplary Bispecific Format 4 Constructs

| | | scFv | | | CH1- | |
|---|---|---|---|---|---|---|
| | Linker- | (Chains | | | CH2- | LC |
| | Fc | 1 | Linker | VH | CH3 | (Chain |
| Name | (Chain1) | and 2) | (Chain2) | (Chain2) | (Chain2) | 3) |
| 4- | GGGGSEP | QVQLVQS | GGGGSGG | QVQLVQS | ASTKGPS | DIQMTQS |
| hOKT3- | KSSDKTH | GAEVKKP | GGS | GAEVKKP | VFPLAPS | PSSLSAS |
| G2(1H11) | TCPPCPA | GASVKVS | (SEQ ID | GASVKVS | SKSTSGG | VGDRVTI |
| | PELLGGP | CKASGYT | NO: | CKASGYT | TAALGCL | TCSASSS |
| | SVFLFPP | FTNYYMH | 107) | FTRYTMH | VKDYFPE | VSYMNWY |
| | KPKDTLM | WVRQAPG | | WVRQAPG | PVTVSWN | QQKPGKA |
| | ISRTPEV | QGLEWMG | | QGLEWMG | SGALTSG | PKRLIYD |
| | TCVVVDV | MINPSGG | | YINPSRG | VHTFPAV | TSKLASG |
| | SHEDPEV | GTSYAQK | | YTNYNQK | LQSSGLY | VPSRFSG |
| | KFNWYVD | FQGRVTM | | FKDRVTL | SLSSVVT | SGSGTDF |
| | GVEVHNA | TRDTSTS | | TTDKSSS | VPSSSLG | TLTISSL |
| | KTKPREE | TVYMELS | | TAYMELS | TQTYICN | QPEDFAT |
| | QYQSTYR | SLRSEDT | | SLRSEDT | VNHKPSN | YYCQQWS |
| | VVSVLTV | AVYYCAR | | AVYYCAR | TKVDKRV | SNPFTFG |
| | LHQDWLN | GNPWELR | | YYDDHYS | EPKSCDK | QGTKLEI |
| | GKEYKCK | LDYWGQG | | LDYWGQG | THTCPPC | KRTVAAP |
| | VSNKALP | TLVTVSS | | TLVTVSS | PAPELLG | SVFIFPP |
| | APIEKTI | GGGGSGG | | (SEQ ID | GPSVFLF | SDEQLKS |
| | SKAKGQP | GGSGGGG | | NO: | PPKPKDT | GTASVVC |
| | REPQVYT | SGGGGSD | | 126) | LMISRTP | LLNNFYP |
| | LPPCREE | IQMTQSP | | | EVTCVVV | REAKVQW |
| | MTKNQVS | SSLSASV | | | DVSHEDP | KVDNALQ |
| | LWCLVKG | GDRVTIT | | | EVKFNWY | SGNSQES |
| | FYPSDIA | CQASQDI | | | VDGVEVH | VTEQDSK |
| | VEWESNG | SNYLNWY | | | NAKTKPR | DSTYSLS |
| | QPENNYK | QQKPGKA | | | EEQYQST | STLTLSK |
| | TTPPVLD | PKLLIYA | | | YRVVSVL | ADYEKHK |
| | SDGSFFL | ASSLQSG | | | TVLHQDW | VYACEVT |
| | YSKLTVD | VPSRFSG | | | LNGKEYK | HQGLSSP |
| | KSRWQQG | SGSGTDF | | | CKVSNKA | VTKSFNR |
| | NVFSCSV | TLTISSL | | | LPAPIEK | GEC |
| | MHEALHN | QPEDFAT | | | TISKAKG | (SEQ ID |
| | HYTQKSL | YYCQQYY | | | QPREPQV | NO: |
| | SLSPGK | SYPFTFG | | | CTLPPSR | 712) |
| | (SEQ ID | PGTKVDI | | | EEMTKNQ | |
| | NO: | K | | | VSLSCAV | |
| | 716) | (SEQ ID | | | KGFYPSD | |
| | | NO: | | | IAVEWES | |
| | | 711) | | | NGQPENN | |
| | | | | | YKTTPPV | |
| | | | | | LDSDGSF | |
| | | | | | FLVSKLT | |
| | | | | | VDKSRWQ | |
| | | | | | QGNVFSC | |
| | | | | | SVMHEAL | |
| | | | | | HNRFTQK | |
| | | | | | SLSLSPG | |
| | | | | | K | |
| | | | | | (SEQ ID | |
| | | | | | NO: | |
| | | | | | 715) | |
| 4- | GGGGSEP | QVQLVQS | GGGGSGG | EVQLVES | ASTKGPS | QAVVTQE |
| anti- | KSSDKTH | GAEVKKP | GGS | GGGLVQP | VFPLAPS | PSLTVSP |
| CD3- | TCPPCPA | GASVKVS | (SEQ ID | GGSLRLS | SKSTSGG | GGTVTLT |
| G2(1H11) | PELLGGP | CKASGYT | NO: | CAASGFT | TAALGCL | CGSSTGA |
| | SVFLFPP | FTNYYMH | 107) | FSTYAMN | VKDYFPE | VTTSNYA |
| | KPKDTLM | WVRQAPG | | WVRQAPG | PVTVSWN | NWVQQKP |
| | ISRTPEV | QGLEWMG | | KGLEWVG | SGALTSG | GKSPRGL |
| | TCVVVDV | MINPSGG | | RIRSKYN | VHTFPAV | IGGTNKR |
| | SHEDPEV | GTSYAQK | | NYATYYA | LQSSGLY | APGVPAR |
| | KFNWYVD | FQGRVTM | | DSVKGRF | SLSSVVT | FSGSLLG |
| | GVEVHNA | TRDTSTS | | TISRDDS | VPSSSLG | GKAALTI |
| | KTKPREE | TVYMELS | | KNTLYLQ | TQTYICN | SGAQPED |
| | QYQSTYR | SLRSEDT | | MNSLRAE | VNHKPSN | EADYYCA |
| | VVSVLTV | AVYYCAR | | DTAVYYC | TKVDKRV | LWYSNHW |
| | LHQDWLN | GNPWELR | | VRHGNFG | EPKSCDK | VFGGGTK |
| | GKEYKCK | LDYWGQG | | DSYVSWF | THTCPPC | LTVLRTV |
| | VSNKALP | TLVTVSS | | AYWGQGT | PAPELLG | AAPSVFI |
| | APIEKTI | GGGGSGG | | LVTVSS | GPSVFLF | FPPSDEQ |
| | SKAKGQP | GGSGGGG | | (SEQ ID | PPKPKDT | LKSGTAS |
| | REPQVYT | SGGGGSD | | NO: | LMISRTP | VVCLLNN |
| | LPPCREE | IQMTQSP | | 132) | EVTCVVV | FYPREAK |
| | MTKNQVS | SSLSASV | | | DVSHEDP | VQWKVDN |
| | LWCLVKG | GDRVTIT | | | EVKFNWY | ALQSGNS |
| | FYPSDIA | CQASQDI | | | VDGVEVH | QESVTEQ |
| | VEWESNG | SNYLNWY | | | NAKTKPR | DSKDSTY |
| | QPENNYK | QQKPGKA | | | EEQYQST | SLSSTLT |
| | TTPPVLD | PKLLIYA | | | YRVVSVL | LSKADYE |
| | SDGSFFL | ASSLQSG | | | TVLHQDW | KHKVYAC |
| | YSKLTVD | VPSRFSG | | | LNGKEYK | EVTHQGL |
| | KSRWQQG | SGSGTDF | | | CKVSNKA | SSPVTKS |
| | NVFSCSV | TLTISSL | | | LPAPIEK | FNRGEC |
| | MHEALHN | QPEDFAT | | | TISKAKG | (SEQ ID |
| | HYTQKSL | YYCQQYY | | | QPREPQV | NO: |
| | SLSPGK | SYPFTFG | | | CTLPPSR | 713) |
| | (SEQ ID | PGTKVDI | | | EEMTKNQ | |
| | NO: | K | | | VSLSCAV | |
| | 716) | (SEQ ID | | | KGFYPSD | |
| | | NO: | | | IAVEWES | |
| | | 711) | | | NGQPENN | |
| | | | | | YKTTPPV | |
| | | | | | LDSDGSF | |
| | | | | | FLVSKLT | |
| | | | | | VDKSRWQ | |
| | | | | | QGNVFSC | |
| | | | | | SVMHEAL | |
| | | | | | HNRFTQK | |
| | | | | | SLSLSPG | |
| | | | | | K | |
| | | | | | (SEQ ID | |
| | | | | | NO: | |
| | | | | | 715) | |
| 4- | GGGGSEP | QVQLVQS | GGGGSGG | QVQLVQS | ASTKGPS | DIQMTQS |
| hOKT3- | KSSDKTH | GAEVKKP | GGS | GAEVKKP | VFPLAPS | PSSLSAS |
| G5(1C12) | TCPPCPA | GSSVKVS | (SEQ ID | GASVKVS | SKSTSGG | VGDRVTI |
| | PELLGGP | CKASGGT | NO: | CKASGYT | TAALGCL | TCSASSS |
| | SVFLFPP | FSNFGVS | 107) | FTRYTMH | VKDYFPE | VSYMNWY |
| | KPKDTLM | WLRQAPG | | WVRQAPG | PVTVSWN | QQKPGKA |
| | ISRTPEV | QGLEWMG | | QGLEWMG | SGALTSG | PKRLIYD |
| | TCVVVDV | GIIPILG | | YINPSRG | VHTFPAV | TSKLASG |
| | SHEDPEV | TANYAQK | | YTNYNQK | LQSSGLY | VPSRFSG |
| | KFNWYVD | FQGRVTI | | FKDRVTL | SLSSVVT | SGSGTDF |
| | GVEVHNA | TADESTS | | TTDKSSS | VPSSSLG | TLTISSL |
| | KTKPREE | TAYMELS | | TAYMELS | TQTYICN | QPEDFAT |
| | QYQSTYR | SLRSEDT | | SLRSEDT | VNHKPSN | YYCQQWS |
| | VVSVLTV | AVYYCAT | | AVYYCAR | TKVDKRV | SNPFTFG |
| | LHQDWLN | PTNSGYY | | YYDDHYS | EPKSCDK | QGTKLEI |
| | GKEYKCK | GPYYYYG | | LDYWGQG | THTCPPC | KRTVAAP |
| | VSNKALP | MDVWGQG | | TLVTVSS | PAPELLG | SVFIFPP |
| | APIEKTI | TTVTVSS | | (SEQ ID | GPSVFLF | SDEQLKS |
| | SKAKGQP | GGGGSGG | | NO: | PPKPKDT | GTASVVC |
| | REPQVYT | GGSGGGG | | 126) | LMISRTP | LLNNFYP |
| | LPPCREE | SGGGGSD | | | EVTCVVV | REAKVQW |
| | MTKNQVS | IQMTQSP | | | DVSHEDP | KVDNALQ |
| | LWCLVKG | SSLSASV | | | EVKFNWY | SGNSQES |
| | FYPSDIA | GDRVTIT | | | VDGVEVH | VTEQDSK |
| | VEWESNG | CRASQSI | | | NAKTKPR | DSTYSLS |
| | QPENNYK | SSWLAWY | | | EEQYQST | STLTLSK |
| | TTPPVLD | QQKPGKA | | | YRVVSVL | ADYEKHK |
| | SDGSFFL | PKLLIYA | | | TVLHQDW | VYACEVT |
| | YSKLTVD | ASTLQSG | | | LNGKEYK | HQGLSSP |
| | KSRWQQG | VPSRFSG | | | CKVSNKA | VTKSFNR |
| | NVFSCSV | SGSGTDF | | | LPAPIEK | GEC |
| | MHEALHN | TLTISSL | | | TISKAKG | (SEQ ID |
| | HYTQKSL | QPEDFAT | | | QPREPQV | NO: |
| | SLSPGK | YYCQQSY | | | CTLPPSR | 712) |
| | (SEQ ID | SIPLTFG | | | EEMTKNQ | |
| | NO: | GGTKVEI | | | VSLSCAV | |
| | 716) | K | | | KGFYPSD | |
| | | (SEQ ID | | | IAVEWES | |
| | | NO: | | | NGQPENN | |
| | | 714) | | | YKTTPPV | |
| | | | | | LDSDGSF | |
| | | | | | FLVSKLT | |
| | | | | | VDKSRWQ | |
| | | | | | QGNVFSC | |
| | | | | | SVMHEAL | |
| | | | | | HNRFTQK | |
| | | | | | SLSLSPG | |
| | | | | | K | |
| | | | | | (SEQ ID | |
| | | | | | NO: | |
| | | | | | 715) | |
| 4- | GGGGSEP | QVQLVQS | GGGGSGG | EVQLVES | ASTKGPS | QAVVTQE |
| anti- | KSSDKTH | GAEVKKP | GGS | GGGLVQP | VFPLAPS | PSLTVSP |
| CD3- | TCPPCPA | GSSVKVS | (SEQ ID | GGSLRLS | SKSTSGG | GGTVTLT |
| G5(1C12) | PELLGGP | CKASGGT | NO: | CAASGFT | TAALGCL | CGSSTGA |
| | SVFLFPP | FSNFGVS | 107) | FSTYAMN | VKDYFPE | VTTSNYA |
| | KPKDTLM | WLRQAPG | | WVRQAPG | PVTVSWN | NWVQQKP |
| | ISRTPEV | QGLEWMG | | KGLEWVG | SGALTSG | GKSPRGL |
| | TCVVVDV | GIIPILG | | RIRSKYN | VHTFPAV | IGGTNKR |
| | SHEDPEV | TANYAQK | | NYATYYA | LQSSGLY | APGVPAR |
| | KFNWYVD | FQGRVTI | | DSVKGRF | SLSSVVT | FSGSLLG |
| | GVEVHNA | TADESTS | | TISRDDS | VPSSSLG | GKAALTI |
| | KTKPREE | TAYMELS | | KNTLYLQ | TQTYICN | SGAQPED |
| | QYQSTYR | SLRSEDT | | MNSLRAE | VNHKPSN | EADYYCA |
| | VVSVLTV | AVYYCAT | | DTAVYYC | TKVDKRV | LWYSNHW |
| | LHQDWLN | PTNSGYY | | VRHGNFG | EPKSCDK | VFGGGTK |
| | GKEYKCK | GPYYYYG | | DSYVSWF | THTCPPC | LTVLRTV |
| | VSNKALP | MDVWGQG | | AYWGQGT | PAPELLG | AAPSVFI |
| | APIEKTI | TTVTVSS | | LVTVSS | GPSVFLF | FPPSDEQ |
| | SKAKGQP | GGGGSGG | | (SEQ ID | PPKPKDT | LKSGTAS |
| | REPQVYT | GGSGGGG | | NO: | LMISRTP | VVCLLNN |
| | LPPCREE | SGGGGSD | | 132) | EVTCVVV | FYPREAK |
| | MTKNQVS | IQMTQSP | | | DVSHEDP | VQWKVDN |
| | LWCLVKG | SSLSASV | | | EVKFNWY | ALQSGNS |
| | FYPSDIA | GDRVTIT | | | VDGVEVH | QESVTEQ |
| | VEWESNG | CRASQSI | | | NAKTKPR | DSKDSTY |
| | QPENNYK | SSWLAWY | | | EEQYQST | SLSSTLT |
| | TTPPVLD | QQKPGKA | | | YRVVSVL | LSKADYE |
| | SDGSFFL | PKLLIYA | | | TVLHQDW | KHKVYAC |
| | YSKLTVD | ASTLQSG | | | LNGKEYK | EVTHQGL |
| | KSRWQQG | VPSRFSG | | | CKVSNKA | SSPVTKS |
| | NVFSCSV | SGSGTDF | | | LPAPIEK | FNRGEC |
| | MHEALHN | TLTISSL | | | TISKAKG | (SEQ ID |
| | HYTQKSL | QPEDFAT | | | QPREPQV | NO: |
| | SLSPGK | YYCQQSY | | | CTLPPSR | 713) |
| | (SEQ ID | SIPLTFG | | | EEMTKNQ | |
| | NO: | GGTKVEI | | | VSLSCAV | |
| | 716) | K | | | KGFYPSD | |
| | | (SEQ ID | | | IAVEWES | |
| | | NO: | | | NGQPENN | |
| | | 714) | | | YKTTPPV | |
| | | | | | LDSDGSF | |
| | | | | | FLVSKLT | |
| | | | | | VDKSRWQ | |
| | | | | | QGNVFSC | |
| | | | | | SVMHEAL | |
| | | | | | HNRFTQK | |
| | | | | | SLSLSPG | |
| | | | | | K | |
| | | | | | (SEQ ID | |
| | | | | | NO: | |
| | | | | | 715) | |

TABLE 37
Exemplary Bispecific Format 5 Constructs

| | | | | | CH1- | |
|---|---|---|---|---|---|---|
| | Hinge- | | | | CH2- | |
| | Fc | scFv | Linker | VH | CH3 | LC |
| Name | (Chain1) | (Chain2) | (Chain2) | (Chain2) | (Chain2) | (Chain 3) |
| 5- | GSEPKSS | QVQLVQS | GGGGSGG | QVQLVQS | ASTKGPS | DIQMTQS |
| hOKT | DKTHTCP | GAEVKKP | GGS | GAEVKKP | VFPLAPS | PSSLSAS |
| 3- | PCPAPEL | GASVKVS | (SEQ | GASVKVS | SKSTSGG | VGDRVTI |
| G2 | LGGPSVF | CKASGYT | ID | CKASGYT | TAALGCL | TCSASSS |
| (1H11) | LFPPKPK | FTNYYMH | NO: | FTRYTMH | VKDYFPE | VSYMNWY |
| | DTLMISR | WVRQAPG | 107) | WVRQAPG | PVTVSWN | QQKPGKA |
| | TPEVTCV | QGLEWMG | | QGLEWMG | SGALTSG | PKRLIYD |
| | VVDVSHE | MINPSGG | | YINPSRG | VHTFPAV | TSKLASG |
| | DPEVKFN | GTSYAQK | | YTNYNQK | LQSSGLY | VPSRFSG |
| | WYVDGVE | FQGRVTM | | FKDRVTL | SLSSVVT | SGSGTDF |
| | VHNAKTK | TRDTSTS | | TTDKSSS | VPSSSLG | TLTISSL |
| | PREEQYQ | TVYMELS | | TAYMELS | TQTYICN | QPEDFAT |
| | STYRVVS | SLRSEDT | | SLRSEDT | VNHKPSN | YYCQQWS |
| | VLTVLHQ | AVYYCAR | | AVYYCAR | TKVDKRV | SNPFTFG |
| | DWLNGKE | GNPWELR | | YYDDHYS | EPKSCDK | QGTKLEI |
| | YKCKVSN | LDYWGQG | | LDYWGQG | THTCPPC | KRTVAAP |
| | KALPAPI | TLVTVSS | | TLVTVSS | PAPELLG | SVFIFPP |
| | EKTISKA | GGGGSGG | | (SEQ ID | GPSVFLF | SDEQLKS |
| | KGQPREP | GGSGGGG | | NO: | PPKPKDT | GTASVVC |
| | QVYTLPP | SGGGGSD | | 126) | LMISRTP | LLNNFYP |
| | CREEMTK | IQMTQSP | | | EVTCVVV | REAKVQW |
| | NQVSLWC | SSLSASV | | | DVSHEDP | KVDNALQ |
| | LVKGFYP | GDRVTIT | | | EVKFNWY | SGNSQES |
| | SDIAVEW | CQASQDI | | | VDGVEVH | VTEQDSK |
| | ESNGQPE | SNYLNWY | | | NAKTKPR | DSTYSLS |
| | NNYKTTP | QQKPGKA | | | EEQYQST | STLTLSK |
| | PVLDSDG | PKLLIYA | | | YRVVSVL | ADYEKHK |
| | SFFLYSK | ASSLQSG | | | TVLHQDW | VYACEVT |
| | LTVDKSR | VPSRFSG | | | LNGKEYK | HQGLSSP |
| | WQQGNVF | SGSGTDF | | | CKVSNKA | VTKSFNR |
| | SCSVMHE | TLTISSL | | | LPAPIEK | GEC |
| | ALHNHYT | QPEDFAT | | | TISKAKG | (SEQ |
| | QKSLSLS | YYCQQYY | | | QPREPQV | ID |
| | PGK | SYPFTFG | | | CTLPPSR | NO: |
| | (SEQ | PGTKVDI | | | EEMTKNQ | 712) |
| | ID | K | | | VSLSCAV | |
| | NO: | (SEQ | | | KGFYPSD | |
| | 717) | ID | | | IAVEWES | |
| | | NO: | | | NGQPENN | |
| | | 711) | | | YKTTPPV | |
| | | | | | LDSDGSF | |
| | | | | | FLVSKLT | |
| | | | | | VDKSRWQ | |
| | | | | | QGNVFSC | |
| | | | | | SVMHEAL | |
| | | | | | HNRFTQK | |
| | | | | | SLSLSPG | |
| | | | | | K | |
| | | | | | (SEQ | |
| | | | | | ID | |
| | | | | | NO: | |
| | | | | | 715) | |
| 5- | GSEPKSS | QVQLVQS | GGGGSGG | EVQLVES | ASTKGPS | QAVVTQE |
| anti- | DKTHTCP | GAEVKKP | GGS | GGGLVQP | VFPLAPS | PSLTVSP |
| CD3- | PCPAPEL | GASVKVS | (SEQ | GGSLRLS | SKSTSGG | GGTVTLT |
| G2 | LGGPSVF | CKASGYT | ID | CAASGFT | TAALGCL | CGSSTGA |
| (1H11) | LFPPKPK | FTNYYMH | NO: | FSTYAMN | VKDYFPE | VTTSNYA |
| | DTLMISR | WVRQAPG | 107) | WVRQAPG | PVTVSWN | NWVQQKP |
| | TPEVTCV | QGLEWMG | | KGLEWVG | SGALTSG | GKSPRGL |
| | VVDVSHE | MINPSGG | | RIRSKYN | VHTFPAV | IGGTNKR |
| | DPEVKFN | GTSYAQK | | NYATYYA | LQSSGLY | APGVPAR |
| | WYVDGVE | FQGRVTM | | DSVKGRF | SLSSVVT | FSGSLLG |
| | VHNAKTK | TRDTSTS | | TISRDDS | VPSSSLG | GKAALTI |
| | PREEQYQ | TVYMELS | | KNTLYLQ | TQTYICN | SGAQPED |
| | STYRVVS | SLRSEDT | | MNSLRAE | VNHKPSN | EADYYCA |
| | VLTVLHQ | AVYYCAR | | DTAVYYC | TKVDKRV | LWYSNHW |
| | DWLNGKE | GNPWELR | | VRHGNFG | EPKSCDK | VFGGGTK |
| | YKCKVSN | LDYWGQG | | DSYVSWF | THTCPPC | LTVLRTV |
| | KALPAPI | TLVTVSS | | AYWGQGT | PAPELLG | AAPSVFI |
| | EKTISKA | GGGGSGG | | LVTVSS | GPSVFLF | FPPSDEQ |
| | KGQPREP | GGSGGGG | | (SEQ | PPKPKDT | LKSGTAS |
| | QVYTLPP | SGGGGSD | | ID | LMISRTP | VVCLLNN |
| | CREEMTK | IQMTQSP | | NO: | EVTCVVV | FYPREAK |
| | NQVSLWC | SSLSASV | | 132) | DVSHEDP | VQWKVDN |
| | LVKGFYP | GDRVTIT | | | EVKFNWY | ALQSGNS |
| | SDIAVEW | CQASQDI | | | VDGVEVH | QESVTEQ |
| | ESNGQPE | SNYLNWY | | | NAKTKPR | DSKDSTY |
| | NNYKTTP | QQKPGKA | | | EEQYQST | SLSSTLT |
| | PVLDSDG | PKLLIYA | | | YRVVSVL | LSKADYE |
| | SFFLYSK | ASSLQSG | | | TVLHQDW | KHKVYAC |
| | LTVDKSR | VPSRFSG | | | LNGKEYK | EVTHQGL |
| | WQQGNVF | SGSGTDF | | | CKVSNKA | SSPVTKS |
| | SCSVMHE | TLTISSL | | | LPAPIEK | FNRGEC |
| | ALHNHYT | QPEDFAT | | | TISKAKG | (SEQ |
| | QKSLSLS | YYCQQYY | | | QPREPQV | ID |
| | PGK | SYPFTFG | | | CTLPPSR | NO: |
| | (SEQ | PGTKVDI | | | EEMTKNQ | 713) |
| | ID | K | | | VSLSCAV | |
| | NO: | (SEQ | | | KGFYPSD | |
| | 717) | ID | | | IAVEWES | |
| | | NO: | | | NGQPENN | |
| | | 711) | | | YKTTPPV | |
| | | | | | LDSDGSF | |
| | | | | | FLVSKLT | |
| | | | | | VDKSRWQ | |
| | | | | | QGNVFSC | |
| | | | | | SVMHEAL | |
| | | | | | HNRFTQK | |
| | | | | | SLSLSPG | |
| | | | | | K | |
| | | | | | (SEQ | |
| | | | | | ID | |
| | | | | | NO: | |
| | | | | | 715) | |
| 5- | GSEPKSS | QVQLVQS | GGGGSGG | QVQLVQS | ASTKGPS | DIQMTQS |
| hOKT | DKTHTCP | GAEVKKP | GGS | GAEVKKP | VFPLAPS | PSSLSAS |
| 3- | PCPAPEL | GSSVKVS | (SEQ | GASVKVS | SKSTSGG | VGDRVTI |
| G5 | LGGPSVF | CKASGGT | ID | CKASGYT | TAALGCL | TCSASSS |
| (1C12) | LFPPKPK | FSNFGVS | NO: | FTRYTMH | VKDYFPE | VSYMNWY |
| | DTLMISR | WLRQAPG | 107) | WVRQAPG | PVTVSWN | QQKPGKA |
| | TPEVTCV | QGLEWMG | | QGLEWMG | SGALTSG | PKRLIYD |
| | VVDVSHE | GIIPILG | | YINPSRG | VHTFPAV | TSKLASG |
| | DPEVKFN | TANYAQK | | YTNYNQK | LQSSGLY | VPSRFSG |
| | WYVDGVE | FQGRVTI | | FKDRVTL | SLSSVVT | SGSGTDF |
| | VHNAKTK | TADESTS | | TTDKSSS | VPSSSLG | TLTISSL |
| | PREEQYQ | TAYMELS | | TAYMELS | TQTYICN | QPEDFAT |
| | STYRVVS | SLRSEDT | | SLRSEDT | VNHKPSN | YYCQQWS |
| | VLTVLHQ | AVYYCAT | | AVYYCAR | TKVDKRV | SNPFTFG |
| | DWLNGKE | PTNSGYY | | YYDDHYS | EPKSCDK | QGTKLEI |
| | YKCKVSN | GPYYYYG | | LDYWGQG | THTCPPC | KRTVAAP |
| | KALPAPI | MDVWGQG | | TLVTVSS | PAPELLG | SVFIFPP |
| | EKTISKA | TTVTVSS | | (SEQ | GPSVFLF | SDEQLKS |
| | KGQPREP | GGGGSGG | | ID | PPKPKDT | GTASVVC |
| | QVYTLPP | GGSGGGG | | NO: | LMISRTP | LLNNFYP |
| | CREEMTK | SGGGGSD | | 126) | EVTCVVV | REAKVQW |
| | NQVSLWC | IQMTQSP | | | DVSHEDP | KVDNALQ |
| | LVKGFYP | SSLSASV | | | EVKFNWY | SGNSQES |
| | SDIAVEW | GDRVTIT | | | VDGVEVH | VTEQDSK |
| | ESNGQPE | CRASQSI | | | NAKTKPR | DSTYSLS |
| | NNYKTTP | SSWLAWY | | | EEQYQST | STLTLSK |
| | PVLDSDG | QQKPGKA | | | YRVVSVL | ADYEKHK |
| | SFFLYSK | PKLLIYA | | | TVLHQDW | VYACEVT |
| | LTVDKSR | ASTLQSG | | | LNGKEYK | HQGLSSP |
| | WQQGNVF | VPSRFSG | | | CKVSNKA | VTKSFNR |
| | SCSVMHE | SGSGTDF | | | LPAPIEK | GEC |
| | ALHNHYT | TLTISSL | | | TISKAKG | (SEQ |
| | QKSLSLS | QPEDFAT | | | QPREPQV | ID |
| | PGK | YYCQQSY | | | CTLPPSR | NO: |
| | (SEQ | SIPLTFG | | | EEMTKNQ | 712) |
| | ID | GGTKVEI | | | VSLSCAV | |
| | NO: | K | | | KGFYPSD | |
| | 717) | (SEQ | | | IAVEWES | |
| | | ID | | | NGQPENN | |
| | | NO: | | | YKTTPPV | |
| | | 714) | | | LDSDGSF | |
| | | | | | FLVSKLT | |
| | | | | | VDKSRWQ | |
| | | | | | QGNVFSC | |
| | | | | | SVMHEAL | |
| | | | | | HNRFTQK | |
| | | | | | SLSLSPG | |
| | | | | | K | |
| | | | | | (SEQ | |
| | | | | | ID | |
| | | | | | NO: | |
| | | | | | 715) | |
| 5- | GSEPKSS | QVQLVQS | GGGGSGG | EVQLVES | ASTKGPS | QAVVTQE |
| anti | DKTHTCP | GAEVKKP | GGS | GGGLVQP | VFPLAPS | PSLTVSP |
| - | PCPAPEL | GSSVKVS | (SEQ | GGSLRLS | SKSTSGG | GGTVTLT |
| CD3- | LGGPSVF | CKASGGT | ID | CAASGFT | TAALGCL | CGSSTGA |
| G5 | LFPPKPK | FSNFGVS | NO: | FSTYAMN | VKDYFPE | VTTSNYA |
| (1C12) | DTLMISR | WLRQAPG | 107) | WVRQAPG | PVTVSWN | NWVQQKP |
| | TPEVTCV | QGLEWMG | | KGLEWVG | SGALTSG | GKSPRGL |
| | VVDVSHE | GIIPILG | | RIRSKYN | VHTFPAV | IGGTNKR |
| | DPEVKFN | TANYAQK | | NYATYYA | LQSSGLY | APGVPAR |
| | WYVDGVE | FQGRVTI | | DSVKGRF | SLSSVVT | FSGSLLG |
| | VHNAKTK | TADESTS | | TISRDDS | VPSSSLG | GKAALTI |
| | PREEQYQ | TAYMELS | | KNTLYLQ | TQTYICN | SGAQPED |
| | STYRVVS | SLRSEDT | | MNSLRAE | VNHKPSN | EADYYCA |
| | VLTVLHQ | AVYYCAT | | DTAVYYC | TKVDKRV | LWYSNHW |
| | DWLNGKE | PTNSGYY | | VRHGNFG | EPKSCDK | VFGGGTK |
| | YKCKVSN | GPYYYYG | | DSYVSWF | THTCPPC | LTVLRTV |
| | KALPAPI | MDVWGQG | | AYWGQGT | PAPELLG | AAPSVFI |
| | EKTISKA | TTVTVSS | | LVTVSS | GPSVFLF | FPPSDEQ |
| | KGQPREP | GGGGSGG | | (SEQ | PPKPKDT | LKSGTAS |
| | QVYTLPP | GGSGGGG | | ID | LMISRTP | VVCLLNN |
| | CREEMTK | SGGGGSD | | NO: | EVTCVVV | FYPREAK |
| | NQVSLWC | IQMTQSP | | 132) | DVSHEDP | VQWKVDN |
| | LVKGFYP | SSLSASV | | | EVKFNWY | ALQSGNS |
| | SDIAVEW | GDRVTIT | | | VDGVEVH | QESVTEQ |
| | ESNGQPE | CRASQSI | | | NAKTKPR | DSKDSTY |
| | NNYKTTP | SSWLAWY | | | EEQYQST | SLSSTLT |
| | PVLDSDG | QQKPGKA | | | YRVVSVL | LSKADYE |
| | SFFLYSK | PKLLIYA | | | TVLHQDW | KHKVYAC |
| | LTVDKSR | ASTLQSG | | | LNGKEYK | EVTHQGL |
| | WQQGNVF | VPSRFSG | | | CKVSNKA | SSPVTKS |
| | SCSVMHE | SGSGTDF | | | LPAPIEK | FNRGEC |
| | ALHNHYT | TLTISSL | | | TISKAKG | (SEQ |
| | QKSLSLS | QPEDFAT | | | QPREPQV | ID |
| | PGK | YYCQQSY | | | CTLPPSR | NO: |
| | (SEQ | SIPLTFG | | | EEMTKNQ | 713) |
| | ID | GGTKVEI | | | VSLSCAV | |
| | NO: | K | | | KGFYPSD | |
| | 717) | (SEQ | | | IAVEWES | |
| | | ID | | | NGQPENN | |
| | | NO: | | | YKTTPPV | |
| | | 714) | | | LDSDGSF | |
| | | | | | FLVSKLT | |
| | | | | | VDKSRWQ | |
| | | | | | QGNVFSC | |
| | | | | | SVMHEAL | |
| | | | | | HNRFTQK | |
| | | | | | SLSLSPG | |
| | | | | | K | |
| | | | | | (SEQ | |
| | | | | | ID | |
| | | | | | NO: | |
| | | | | | 715) | |

TABLE 38
Exemplary Bispecific Format 6 Constructs

| | | | | CH1_ | |
|---|---|---|---|---|---|
| | | | | CH2_ | |
| | scFv | | VH | CH3 | LC |
| | (Chain | | (Chain 1 | (Chain 1 | (Chains 3 |
| Name | 1 and 2) | Linker | and 2) | and 2) | and 4)) |
| 6-hOKT3- | QVQLVQSGAE | GGGGSGGGGS | QVQLVQSGAE | ASTKGPSVFP | DIQMTQSPSS |
| G2(1H11) | VKKPGASVKV | (SEQ ID | VKKPGASVKV | LAPSSKSTSG | LSASVGDRVT |
| | SCKASGYTFT | NO: | SCKASGYTFT | GTAALGCLVK | ITCSASSSVS |
| | NYYMHWVRQA | 107) | RYTMHWVRQA | DYFPEPVTVS | YMNWYQQKPG |
| | PGQGLEWMGM | | PGQGLEWMGY | WNSGALTSGV | KAPKRLIYDT |
| | INPSGGGTSY | | INPSRGYTNY | HTFPAVLQSS | SKLASGVPSR |
| | AQKFQGRVTM | | NQKFKDRVTL | GLYSLSSVVT | FSGSGSGTDF |
| | TRDTSTSTVY | | TTDKSSSTAY | VPSSSLGTQT | TLTISSLQPE |
| | MELSSLRSED | | MELSSLRSED | YICNVNHKPS | DFATYYCQQW |
| | TAVYYCARGN | | TAVYYCARYY | NTKVDKRVEP | SSNPFTFGQG |
| | PWELRLDYWG | | DDHYSLDYWG | KSCDKTHTCP | TKLEIKRTVA |
| | QGTLVTVSSG | | QGTLVTVSS | PCPAPELLGG | APSVFIFPPS |
| | GGGSGGGGSG | | (SEQ ID | PSVFLFPPKP | DEQLKSGTAS |
| | GGGSGGGGSD | | NO: | KDTLMISRTP | VVCLLNNFYP |
| | IQMTQSPSSL | | 126) | EVTCVVVDVS | REAKVQWKVD |
| | SASVGDRVTI | | | HEDPEVKFNW | NALQSGNSQE |
| | TCQASQDISN | | | YVDGVEVHNA | SVTEQDSKDS |
| | YLNWYQQKPG | | | KTKPREEQYQ | TYSLSSTLTL |
| | KAPKLLIYAA | | | STYRVVSVLT | SKADYEKHKV |
| | SSLQSGVPSR | | | VLHQDWLNGK | YACEVTHQGL |
| | FSGSGSGTDF | | | EYKCKVSNKA | SSPVTKSFNR |
| | TLTISSLQPE | | | LPAPIEKTIS | GEC |
| | DFATYYCQQY | | | KAKGQPREPQ | (SEQ ID |
| | YSYPFTFGPG | | | VYTLPPSREE | NO: |
| | TKVDIK | | | MTKNQVSLTC | 712) |
| | (SEQ ID | | | LVKGFYPSDI | |
| | NO: | | | AVEWESNGQP | |
| | 711) | | | ENNYKTTPPV | |
| | | | | LDSDGSFFLY | |
| | | | | SKLTVDKSRW | |
| | | | | QQGNVFSCSV | |
| | | | | MHEALHNHYT | |
| | | | | QKSLSLSPGK | |
| | | | | (SEQ ID | |
| | | | | NO: | |
| | | | | 710) | |
| 6-anti-CD3- | QVQLVQSGAE | GGGGSGGGGS | EVQLVESGGG | ASTKGPSVFP | QAVVTQEPSL |
| G2(1H11) | VKKPGASVKV | (SEQ ID | LVQPGGSLRL | LAPSSKSTSG | TVSPGGTVTL |
| | SCKASGYTFT | NO: | SCAASGFTFS | GTAALGCLVK | TCGSSTGAVT |
| | NYYMHWVRQA | 107) | TYAMNWVRQA | DYFPEPVTVS | TSNYANWVQQ |
| | PGQGLEWMGM | | PGKGLEWVGR | WNSGALTSGV | KPGKSPRGLI |
| | INPSGGGTSY | | IRSKYNNYAT | HTFPAVLQSS | GGTNKRAPGV |
| | AQKFQGRVTM | | YYADSVKGRF | GLYSLSSVVT | PARFSGSLLG |
| | TRDTSTSTVY | | TISRDDSKNT | VPSSSLGTQT | GKAALTISGA |
| | MELSSLRSED | | LYLQMNSLRA | YICNVNHKPS | QPEDEADYYC |
| | TAVYYCARGN | | EDTAVYYCVR | NTKVDKRVEP | ALWYSNHWVF |
| | PWELRLDYWG | | HGNFGDSYVS | KSCDKTHTCP | GGGTKLTVLR |
| | QGTLVTVSSG | | WFAYWGQGTL | PCPAPELLGG | TVAAPSVFIF |
| | GGGSGGGGSG | | VTVSS | PSVFLFPPKP | PPSDEQLKSG |
| | GGGSGGGGSD | | (SEQ ID | KDTLMISRTP | TASVVCLLNN |
| | IQMTQSPSSL | | NO: | EVTCVVVDVS | FYPREAKVQW |
| | SASVGDRVTI | | 132) | HEDPEVKFNW | KVDNALQSGN |
| | TCQASQDISN | | | YVDGVEVHNA | SQESVTEQDS |
| | YLNWYQQKPG | | | KTKPREEQYQ | KDSTYSLSST |
| | KAPKLLIYAA | | | STYRVVSVLT | LTLSKADYEK |
| | SSLQSGVPSR | | | VLHQDWLNGK | HKVYACEVTH |
| | FSGSGSGTDF | | | EYKCKVSNKA | QGLSSPVTKS |
| | TLTISSLQPE | | | LPAPIEKTIS | FNRGEC |
| | DFATYYCQQY | | | KAKGQPREPQ | (SEQ ID |
| | YSYPFTFGPG | | | VYTLPPSREE | NO: |
| | TKVDIK | | | MTKNQVSLTC | 713) |
| | (SEQ ID | | | LVKGFYPSDI | |
| | NO: | | | AVEWESNGQP | |
| | 711) | | | ENNYKTTPPV | |
| | | | | LDSDGSFFLY | |
| | | | | SKLTVDKSRW | |
| | | | | QQGNVFSCSV | |
| | | | | MHEALHNHYT | |
| | | | | QKSLSLSPGK | |
| | | | | (SEQ ID | |
| | | | | NO: | |
| | | | | 710) | |
| 6-hOKT3- | QVQLVQSGAE | GGGGSGGGGS | QVQLVQSGAE | ASTKGPSVFP | DIQMTQSPSS |
| G5(1C12) | VKKPGSSVKV | (SEQ ID | VKKPGASVKV | LAPSSKSTSG | LSASVGDRVT |
| | SCKASGGTFS | NO: | SCKASGYTFT | GTAALGCLVK | ITCSASSSVS |
| | NFGVSWLRQA | 107) | RYTMHWVRQA | DYFPEPVTVS | YMNWYQQKPG |
| | PGQGLEWMGG | | PGQGLEWMGY | WNSGALTSGV | KAPKRLIYDT |
| | IIPILGTANY | | INPSRGYTNY | HTFPAVLQSS | SKLASGVPSR |
| | AQKFQGRVTI | | NQKFKDRVTL | GLYSLSSVVT | FSGSGSGTDF |
| | TADESTSTAY | | TTDKSSSTAY | VPSSSLGTQT | TLTISSLOPE |
| | MELSSLRSED | | MELSSLRSED | YICNVNHKPS | DFATYYCQQW |
| | TAVYYCATPT | | TAVYYCARYY | NTKVDKRVEP | SSNPFTFGQG |
| | NSGYYGPYYY | | DDHYSLDYWG | KSCDKTHTCP | TKLEIKRTVA |
| | YGMDVWGQGT | | QGTLVTVSS | PCPAPELLGG | APSVFIFPPS |
| | TVTVSSGGGG | | (SEQ ID | PSVFLFPPKP | DEQLKSGTAS |
| | SGGGGSGGGG | | NO: | KDTLMISRTP | VVCLLNNFYP |
| | SGGGGSDIQM | | 126) | EVTCVVVDVS | REAKVQWKVD |
| | TQSPSSLSAS | | | HEDPEVKFNW | NALQSGNSQE |
| | VGDRVTITCR | | | YVDGVEVHNA | SVTEQDSKDS |
| | ASQSISSWLA | | | KTKPREEQYQ | TYSLSSTLTL |
| | WYQQKPGKAP | | | STYRVVSVLT | SKADYEKHKV |
| | KLLIYAASTL | | | VLHQDWLNGK | YACEVTHQGL |
| | QSGVPSRFSG | | | EYKCKVSNKA | SSPVTKSFNR |
| | SGSGTDFTLT | | | LPAPIEKTIS | GEC |
| | ISSLQPEDFA | | | KAKGQPREPQ | (SEQ ID |
| | TYYCQQSYSI | | | VYTLPPSREE | NO: |
| | PLTFGGGTKV | | | MTKNQVSLTC | 712) |
| | EIK | | | LVKGFYPSDI | |
| | (SEQ ID | | | AVEWESNGQP | |
| | NO: | | | ENNYKTTPPV | |
| | 714) | | | LDSDGSFFLY | |
| | | | | SKLTVDKSRW | |
| | | | | QQGNVFSCSV | |
| | | | | MHEALHNHYT | |
| | | | | QKSLSLSPGK | |
| | | | | (SEQ ID | |
| | | | | NO: | |
| | | | | 710) | |
| 6-anti-CD3- | QVQLVQSGAE | GGGGSGGGGS | EVQLVESGGG | ASTKGPSVFP | QAVVTQEPSL |
| G5(1C12) | VKKPGSSVKV | (SEQ ID | LVQPGGSLRL | LAPSSKSTSG | TVSPGGTVTL |
| | SCKASGGTFS | NO: | SCAASGFTFS | GTAALGCLVK | TCGSSTGAVT |
| | NFGVSWLRQA | 107) | TYAMNWVRQA | DYFPEPVTVS | TSNYANWVQQ |
| | PGQGLEWMGG | | PGKGLEWVGR | WNSGALTSGV | KPGKSPRGLI |
| | IIPILGTANY | | IRSKYNNYAT | HTFPAVLQSS | GGTNKRAPGV |
| | AQKFQGRVTI | | YYADSVKGRF | GLYSLSSVVT | PARFSGSLLG |
| | TADESTSTAY | | TISRDDSKNT | VPSSSLGTQT | GKAALTISGA |
| | MELSSLRSED | | LYLQMNSLRA | YICNVNHKPS | QPEDEADYYC |
| | TAVYYCATPT | | EDTAVYYCVR | NTKVDKRVEP | ALWYSNHWVF |
| | NSGYYGPYYY | | HGNFGDSYVS | KSCDKTHTCP | GGGTKLTVLR |
| | YGMDVWGQGT | | WFAYWGQGTL | PCPAPELLGG | TVAAPSVFIF |
| | TVTVSSGGGG | | VTVSS | PSVFLFPPKP | PPSDEQLKSG |
| | SGGGGSGGGG | | (SEQ ID | KDTLMISRTP | TASVVCLLNN |
| | SGGGGSDIQM | | NO: | EVTCVVVDVS | FYPREAKVQW |
| | TQSPSSLSAS | | 132) | HEDPEVKFNW | KVDNALQSGN |
| | VGDRVTITCR | | | YVDGVEVHNA | SQESVTEQDS |
| | ASQSISSWLA | | | KTKPREEQYQ | KDSTYSLSST |
| | WYQQKPGKAP | | | STYRVVSVLT | LTLSKADYEK |
| | KLLIYAASTL | | | VLHQDWLNGK | HKVYACEVTH |
| | QSGVPSRFSG | | | EYKCKVSNKA | QGLSSPVTKS |
| | SGSGTDFTLT | | | LPAPIEKTIS | FNRGEC |
| | ISSLQPEDFA | | | KAKGQPREPQ | (SEQ ID |
| | TYYCQQSYSI | | | VYTLPPSREE | NO: |
| | PLTFGGGTKV | | | MTKNQVSLTC | 713) |
| | EIK | | | LVKGFYPSDI | |
| | (SEQ ID | | | AVEWESNGQP | |
| | NO: | | | ENNYKTTPPV | |
| | 714) | | | LDSDGSFFLY | |
| | | | | SKLTVDKSRW | |
| | | | | QQGNVFSCSV | |
| | | | | MHEALHNHYT | |
| | | | | QKSLSLSPGK | |
| | | | | (SEQ ID | |
| | | | | NO: | |
| | | | | 710) | |

antibodies with engineered disulfide
bond (DSB)

| | | | | | CH1- | |
|---|---|---|---|---|---|---|
| | | scFv | | | CH2- | |
| | Linker-Fc | (Chains 1 | Linker | | CH3 | LC |
| Name | (Chain1) | and 2) | (Chain2) | VH (Chain2) | (Chain2) | (Chain 3) |
| 4- | GGGGSEPKSS | QVQLVQSGAE | GGGGSGGGGS | QVQLVQSGAE | ASTKGPSVFP | DIQMTQSPSS |
| hOKT | DKTHTCPPCP | VKKPGASVKV | (SEQ ID | VKKPGASVKV | LAPSSKSTSG | LSASVGDRVT |
| 3- | APELLGGPSV | SCKASGYTFT | NO: | SCKASGYTFT | GTAALGCLVK | ITCSASSSVS |
| G2 | FLFPPKPKDT | NYYMHWVRQA | 107 | RYTMHWVRQA | DYFPEPVTVS | YMNWYQQKPG |
| (1H11) | LMISRTPEVT | PGQCLEWMGM | | PGQGLEWMGY | WNSGALTSGV | KAPKRLIYDT |
| | CVVVDVSHED | INPSGGGTSY | | INPSRGYTNY | HTFPAVLOSS | SKLASGVPSR |
| | PEVKFNWYVD | AQKFQGRVTM | | NQKFKDRVTL | GLYSLSSVVT | FSGSGSGTDF |
| | GVEVHNAKTK | TRDTSTSTVY | | TTDKSSSTAY | VPSSSLGTQT | TLTISSLQPE |
| | PREEQYQSTY | MELSSLRSED | | MELSSLRSED | YICNVNHKPS | DFATYYCQQW |
| | RVVSVLTVLH | TAVYYCARGN | | TAVYYCARYY | NTKVDKRVEP | SSNPFTFGQG |
| | QDWLNGKEYK | PWELRLDYWG | | DDHYSLDYWG | KSCDKTHTCP | TKLEIKRTVA |
| | CKVSNKALPA | QGTLVTVSSG | | QGTLVTVSS | PCPAPELLGG | APSVFIFPPS |
| | PIEKTISKAK | GGGSGGGGSG | | (SEQ ID | PSVFLFPPKP | DEQLKSGTAS |
| | GQPREPQVYT | GGGSGGGGSD | | NO: | KDTLMISRTP | VVCLLNNFYP |
| | LPPCREEMTK | IQMTQSPSSL | | 126) | EVTCVVVDVS | REAKVQWKVD |
| | NQVSLWCLVK | SASVGDRVTI | | | HEDPEVKFNW | NALQSGNSQE |
| | GFYPSDIAVE | TCQASQDISN | | | YVDGVEVHNA | SVTEQDSKDS |
| | WESNGQPENN | YLNWYQQKPG | | | KTKPREEQYQ | TYSLSSTLTL |
| | YKTTPPVLDS | KAPKLLIYAA | | | STYRVVSVLT | SKADYEKHKV |
| | DGSFFLYSKL | SSLQSGVPSR | | | VLHQDWLNGK | YACEVTHQGL |
| | TVDKSRWQQG | FSGSGSGTDF | | | EYKCKVSNKA | SSPVTKSFNR |
| | NVFSCSVMHE | TLTISSLQPE | | | LPAPIEKTIS | GEC |
| | ALHNHYTQKS | DFATYYCQQY | | | KAKGQPREPQ | (SEQ ID |
| | LSLSPGK | YSYPFTFGCG | | | VCTLPPSREE | NO: |
| | (SEQ ID | TKVDIK | | | MTKNQVSLSC | 712) |
| | NO: | (SEQ ID | | | AVKGFYPSDI | |
| | 716) | NO: | | | AVEWESNGQP | |
| | | 718) | | | ENNYKTTPPV | |
| | | | | | LDSDGSFFLV | |
| | | | | | SKLTVDKSRW | |
| | | | | | QQGNVFSCSV | |
| | | | | | MHEALHNRFT | |
| | | | | | QKSLSLSPGK | |
| | | | | | (SEQ ID | |
| | | | | | NO: | |
| | | | | | 715) | |
| 4- | GGGGSEPKSS | QVQLVQSGAE | GGGGSGGGGS | QVQLVQSGAE | ASTKGPSVFP | DIQMTQSPSS |
| hOKT | DKTHTCPPCP | VKKPGSSVKV | (SEQ ID | VKKPGASVKV | LAPSSKSTSG | LSASVGDRVT |
| 3- | APELLGGPSV | SCKASGGTFS | NO: | SCKASGYTFT | GTAALGCLVK | ITCSASSSVS |
| G5 | FLFPPKPKDT | NFGVSWLRQA | 107) | RYTMHWVRQA | DYFPEPVTVS | YMNWYQQKPG |
| (1C12) | LMISRTPEVT | PGQCLEWMGG | | PGQGLEWMGY | WNSGALTSGV | KAPKRLIYDT |
| | CVVVDVSHED | IIPILGTANY | | INPSRGYTNY | HTFPAVLQSS | SKLASGVPSR |
| | PEVKFNWYVD | AQKFQGRVTI | | NQKFKDRVTL | GLYSLSSVVT | FSGSGSGTDF |
| | GVEVHNAKTK | TADESTSTAY | | TTDKSSSTAY | VPSSSLGTQT | TLTISSLQPE |
| | PREEQYQSTY | MELSSLRSED | | MELSSLRSED | YICNVNHKPS | DFATYYCQQW |
| | RVVSVLTVLH | TAVYYCATPT | | TAVYYCARYY | NTKVDKRVEP | SSNPFTFGQG |
| | QDWLNGKEYK | NSGYYGPYYY | | DDHYSLDYWG | KSCDKTHTCP | TKLEIKRTVA |
| | CKVSNKALPA | YGMDVWGQGT | | QGTLVTVSS | PCPAPELLGG | APSVFIFPPS |
| | PIEKTISKAK | TVTVSSGGGG | | (SEQ ID | PSVFLFPPKP | DEQLKSGTAS |
| | GQPREPQVYT | SGGGGSGGGG | | NO: | KDTLMISRTP | VVCLLNNFYP |
| | LPPCREEMTK | SGGGGSDIQM | | 126) | EVTCVVVDVS | REAKVQWKVD |
| | NQVSLWCLVK | TQSPSSLSAS | | | HEDPEVKFNW | NALQSGNSQE |
| | GFYPSDIAVE | VGDRVTITCR | | | YVDGVEVHNA | SVTEQDSKDS |
| | WESNGQPENN | ASQSISSWLA | | | KTKPREEQYQ | TYSLSSTLTL |
| | YKTTPPVLDS | WYQQKPGKAP | | | STYRVVSVLT | SKADYEKHKV |
| | DGSFFLYSKL | KLLIYAASTL | | | VLHQDWLNGK | YACEVTHQGL |
| | TVDKSRWQQG | QSGVPSRFSG | | | EYKCKVSNKA | SSPVTKSFNR |
| | NVFSCSVMHE | SGSGTDFTLT | | | LPAPIEKTIS | GEC |
| | ALHNHYTQKS | ISSLOPEDFA | | | KAKGQPREPQ | (SEQ ID |
| | LSLSPGK | TYYCQQSYSI | | | VCTLPPSREE | NO: |
| | (SEQ ID | PLTFGGGTKV | | | MTKNQVSLSC | 712) |
| | NO: | EIK | | | AVKGFYPSDI | |
| | 716) | (SEQ ID | | | AVEWESNGQP | |
| | | NO: | | | ENNYKTTPPV | |
| | | 719) | | | LDSDGSFFLV | |
| | | | | | SKLTVDKSRW | |
| | | | | | QQGNVFSCSV | |
| | | | | | MHEALHNRFT | |
| | | | | | QKSLSLSPGK | |
| | | | | | (SEQ ID | |
| | | | | | NO: | |
| | | | | | 715) | |

bold text indicates the engineered cysteine residue

TABLE 42

| | | | | | CH1- | |
|---|---|---|---|---|---|---|
| | | ABR | | | hinge- | |
| | Linker-Fc | (Chains 1 | Linker | VH | CH2-CH3 | LC |
| Name | (Chain1) | and 2) | (Chain2) | (Chain2) | (Chain2) | (Chain 3) |
| D-1H11- | GGCGGG | QVQLVQS | GGCGGG | QVQLVQS | ASTKGPS | DIQMTQS |
| LL- | GSGSEPK | GAEVKKP | GSGSGGG | GAEVKKP | VFPLAPSS | PSSLSASV |
| GGCxhOKT3 | SSDKTHT | GASVKVS | GS (SEQ | GASVKVS | KSTSGGT | GDRVTIT |
| | CPPCPAP | CKASGYT | ID NO: | CKASGYT | AALGCLV | CSASSSV |
| | EFEGGPS | FTNYYM | 407) | FTRYTMH | KDYFPEP | SYMNWY |
| | VFLFPPKP | HWVRQA | | WVRQAP | VTVSWNS | QQKPGK |
| | KDTLMIS | PGQGLE | | GQGLEW | GALTSGV | APKRLIY |
| | RTPEVTC | WMGMIN | | MGYINPS | HTFPAVL | DTSKLAS |
| | VVVDVS | PSGGGTS | | RGYTNY | QSSGLYS | GVPSRFS |
| | HEDPEVK | YAQKFQ | | NQKFKDR | LSSVVTV | GSGSGTD |
| | FNWYVD | GRVTMT | | VTLTTDK | PSSSLGT | FTLTISSL |
| | GVEVHN | RDTSTST | | SSSTAYM | QTYICNV | QPEDFAT |
| | AKTKPRE | VYMELSS | | ELSSLRSE | NHKPSNT | YYCQQW |
| | EQYNSTY | LRSEDTA | | DTAVYY | KVDKRV | SSNPFTFG |
| | RVVSVLT | VYYCAR | | CARYYD | EPKSCDK | QGTKLEI |
| | VLHQDW | GNPWELR | | DHYSLDY | THTCPPC | KRTVAAP |
| | LNGKEYK | LDYWGQ | | WGQGTL | PAPELLG | SVFIFPPS |
| | CKVSNKA | GTLVTVS | | VTVSS | GPSVFLFP | DEQLKSG |
| | LPASIEKT | SGGGGSG | | (SEQ ID | PKPKDTL | TASVVCL |
| | ISKAKGQ | GGGSDIQ | | NO: 126) | MISRTPE | LNNFYPR |
| | PREPQVY | MTQSPSS | | | VTCVVV | EAKVQW |
| | TLPPCRE | LSASVGD | | | DVSHEDP | KVDNAL |
| | EMTKNQ | RVTITCQ | | | EVKFNW | QSGNSQE |
| | VSLWCLV | ASQDISN | | | YVDGVE | SVTEQDS |
| | KGFYPSD | YLNWYQ | | | VHNAKT | KDSTYSL |
| | IAVEWES | QKPGKAP | | | KPREEQY | SSTLTLSK |
| | NGQPENN | KLLIYAA | | | NSTYRVV | ADYEKH |
| | YKTTPPV | SSLQSGV | | | SVLTVLH | KVYACE |
| | LDSDGSF | PSRFSGS | | | QDWLNG | VTHQGLS |
| | FLYSKLT | GSGTDFT | | | KEYKCK | SPVTKSF |
| | VDKSRW | LTISSLQP | | | VSNKALP | NRGEC |
| | QQGNVFS | EDFATYY | | | APIEKTIS | (SEQ ID |
| | CSVMHE | CQQYYSY | | | KAKGQPR | NO: 712) |
| | ALHNHYT | PFTFGPG | | | EPQVCTL | |
| | QKSLSLS | TKVDIK | | | PPSREEM | |
| | PGK (SEQ | (SEQ ID | | | TKNQVSL | |
| | ID NO: | NO: 721) | | | SCAVKGF | |
| | 720) | | | | YPSDIAV | |
| | | | | | EWESNG | |
| | | | | | QPENNYK | |
| | | | | | TTPPVLD | |
| | | | | | SDGSFFL | |
| | | | | | VSKLTVD | |
| | | | | | KSRWQQ | |
| | | | | | GNVFSCS | |
| | | | | | VMHEAL | |
| | | | | | HNHYTQ | |
| | | | | | KSLSLSP | |
| | | | | | GK (SEQ | |
| | | | | | ID NO: | |
| | | | | | 722) | |

TABLE 43

| | | | | | CH1- | |
|---|---|---|---|---|---|---|
| | | ABR | | | hinge- | |
| | Linker-Fc | (Chains 1 | Linker | VH | CH2-CH3 | LC |
| Name | (Chain1) | and 2) | (Chain2) | (Chain2) | (Chain2) | (Chain 3) |
| D-1H11- | GGCGSEP | QVQLVQS | GGCGGG | QVQLVQS | ASTKGPS | DIQMTQS |
| LS- | KSSDKTH | GAEVKKP | GSGSGGG | GAEVKKP | VFPLAPSS | PSSLSASV |
| GGCxhOKT3 | TCPPCPA | GASVKVS | GS (SEQ | GASVKVS | KSTSGGT | GDRVTIT |
| | PEFEGGP | CKASGYT | ID NO: | CKASGYT | AALGCLV | CSASSSV |
| | SVFLFPPK | FTNYYM | 407) | FTRYTMH | KDYFPEP | SYMNWY |
| | PKDTLMI | HWVRQA | | WVRQAP | VTVSWNS | QQKPGK |
| | SRTPEVT | PGQGLE | | GQGLEW | GALTSGV | APKRLIY |
| | CVVVDVS | WMGMIN | | MGYINPS | HTFPAVL | DTSKLAS |
| | HEDPEVK | PSGGGTS | | RGYTNY | QSSGLYS | GVPSRFS |
| | FNWYVD | YAQKFQ | | NQKFKDR | LSSVVTV | GSGSGTD |
| | GVEVHN | GRVTMT | | VTLTTDK | PSSSLGT | FTLTISSL |
| | AKTKPRE | RDTSTST | | SSSTAYM | QTYICNV | QPEDFAT |
| | EQYNSTY | VYMELSS | | ELSSLRSE | NHKPSNT | YYCQQW |
| | RVVSVLT | LRSEDTA | | DTAVYY | KVDKRV | SSNPFTFG |
| | VLHQDW | VYYCAR | | CARYYD | EPKSCDK | QGTKLEI |
| | LNGKEYK | GNPWELR | | DHYSLDY | THTCPPC | KRTVAAP |
| | CKVSNKA | LDYWGQ | | WGQGTL | PAPELLG | SVFIFPPS |
| | LPASIEKT | GTLVTVS | | VTVSS | GPSVFLFP | DEQLKSG |
| | ISKAKGQ | SGGGGSG | | (SEQ ID | PKPKDTL | TASVVCL |
| | PREPQVY | GGGSDIQ | | NO: 126) | MISRTPE | LNNFYPR |
| | TLPPCRE | MTQSPSS | | | VTCVVV | EAKVQW |
| | EMTKNQ | LSASVGD | | | DVSHEDP | KVDNAL |
| | VSLWCLV | RVTITCQ | | | EVKFNW | QSGNSQE |
| | KGFYPSD | ASQDISN | | | YVDGVE | SVTEQDS |
| | IAVEWES | YLNWYQ | | | VHNAKT | KDSTYSL |
| | NGQPENN | QKPGKAP | | | KPREEQY | SSTLTLSK |
| | YKTTPPV | KLLIYAA | | | NSTYRVV | ADYEKH |
| | LDSDGSF | SSLQSGV | | | SVLTVLH | KVYACE |
| | FLYSKLT | PSRFSGS | | | QDWLNG | VTHQGLS |
| | VDKSRW | GSGTDFT | | | KEYKCK | SPVTKSF |
| | QQGNVFS | LTISSLQP | | | VSNKALP | NRGEC |
| | CSVMHE | EDFATYY | | | APIEKTIS | (SEQ ID |
| | ALHNHYT | CQQYYSY | | | KAKGQPR | NO: 712) |
| | QKSLSLS | PFTFGPG | | | EPQVCTL | |
| | PGK (SEQ | TKVDIK | | | PPSREEM | |
| | ID NO: | (SEQ ID | | | TKNQVSL | |
| | 723) | NO: 721) | | | SCAVKGF | |
| | | | | | YPSDIAV | |
| | | | | | EWESNG | |
| | | | | | QPENNYK | |
| | | | | | TTPPVLD | |
| | | | | | SDGSFFL | |
| | | | | | VSKLTVD | |
| | | | | | KSRWQQ | |
| | | | | | GNVFSCS | |
| | | | | | VMHEAL | |
| | | | | | HNHYTQ | |
| | | | | | KSLSLSP | |
| | | | | | GK (SEQ | |
| | | | | | ID NO: | |
| | | | | | 722) | |

TABLE 44

| | | | | | CH1- | |
|---|---|---|---|---|---|---|
| | | ABR | | | hinge- | |
| | Linker-Fc | (Chains 1 | Linker | VH | CH2-CH3 | LC |
| Name | (Chain1) | and 2) | (Chain2) | (Chain2) | (Chain2) | (Chain 3) |
| D-1H11- | GGCGGG | QVQLVQS | GGCGS | QVQLVQS | ASTKGPS | DIQMTQS |
| SL- | GSGSEPK | GAEVKKP | (SEQ ID | GAEVKKP | VFPLAPSS | PSSLSASV |
| GGCxhOKT3 | SSDKTHT | GASVKVS | NO: 409) | GASVKVS | KSTSGGT | GDRVTIT |
| | CPPCPAP | CKASGYT | | CKASGYT | AALGCLV | CSASSSV |
| | EFEGGPS | FTNYYM | | FTRYTMH | KDYFPEP | SYMNWY |
| | VFLFPPKP | HWVRQA | | WVRQAP | VTVSWNS | QQKPGK |
| | KDTLMIS | PGQGLE | | GQGLEW | GALTSGV | APKRLIY |
| | RTPEVTC | WMGMIN | | MGYINPS | HTFPAVL | DTSKLAS |
| | VVVDVS | PSGGGTS | | RGYTNY | QSSGLYS | GVPSRFS |
| | HEDPEVK | YAQKFQ | | NQKFKDR | LSSVVTV | GSGSGTD |
| | FNWYVD | GRVTMT | | VTLTTDK | PSSSLGT | FTLTISSL |
| | GVEVHN | RDTSTST | | SSSTAYM | QTYICNV | QPEDFAT |
| | AKTKPRE | VYMELSS | | ELSSLRSE | NHKPSNT | YYCQQW |
| | EQYNSTY | LRSEDTA | | DTAVYY | KVDKRV | SSNPFTFG |
| | RVVSVLT | VYYCAR | | CARYYD | EPKSCDK | QGTKLEI |
| | VLHQDW | GNPWELR | | DHYSLDY | THTCPPC | KRTVAAP |
| | LNGKEYK | LDYWGQ | | WGQGTL | PAPELLG | SVFIFPPS |
| | CKVSNKA | GTLVTVS | | VTVSS | GPSVFLFP | DEQLKSG |
| | LPASIEKT | SGGGGSG | | (SEQ ID | PKPKDTL | TASVVCL |
| | ISKAKGQ | GGGSDIQ | | NO: 126) | MISRTPE | LNNFYPR |
| | PREPQVY | MTQSPSS | | | VTCVVV | EAKVQW |
| | TLPPCRE | LSASVGD | | | DVSHEDP | KVDNAL |
| | EMTKNQ | RVTITCQ | | | EVKFNW | QSGNSQE |
| | VSLWCLV | ASQDISN | | | YVDGVE | SVTEQDS |
| | KGFYPSD | YLNWYQ | | | VHNAKT | KDSTYSL |
| | IAVEWES | QKPGKAP | | | KPREEQY | SSTLTLSK |
| | NGQPENN | KLLIYAA | | | NSTYRVV | ADYEKH |
| | YKTTPPV | SSLQSGV | | | SVLTVLH | KVYACE |
| | LDSDGSF | PSRFSGS | | | QDWLNG | VTHQGLS |
| | FLYSKLT | GSGTDFT | | | KEYKCK | SPVTKSF |
| | VDKSRW | LTISSLQP | | | VSNKALP | NRGEC |
| | QQGNVFS | EDFATYY | | | APIEKTIS | (SEQ ID |
| | CSVMHE | CQQYYSY | | | KAKGQPR | NO: 712) |
| | ALHNHYT | PFTFGPG | | | EPQVCTL | |
| | QKSLSLS | TKVDIK | | | PPSREEM | |
| | PGK (SEQ | (SEQ ID | | | TKNQVSL | |
| | ID NO: | NO: 721) | | | SCAVKGF | |
| | 720) | | | | YPSDIAV | |
| | | | | | EWESNG | |
| | | | | | QPENNYK | |
| | | | | | TTPPVLD | |
| | | | | | SDGSFFL | |
| | | | | | VSKLTVD | |
| | | | | | KSRWQQ | |
| | | | | | GNVFSCS | |
| | | | | | VMHEAL | |
| | | | | | HNHYTQ | |
| | | | | | KSLSLSP | |
| | | | | | GK (SEQ | |
| | | | | | ID NO: | |
| | | | | | 722) | |

TABLE 45

| | | | | | CH1- | |
|---|---|---|---|---|---|---|
| | | ABR | | | hinge- | |
| | Linker-Fc | (Chains 1 | Linker | VH | CH2-CH3 | LC |
| Name | (Chain1) | and 2) | (Chain2) | (Chain2) | (Chain2) | (Chain 3) |
| D-1H11- | GGGCGG | QVQLVQS | GGGCGG | QVQLVQS | ASTKGPS | DIQMTQS |
| LL- | GSGSEPK | GAEVKKP | GSGSGGG | GAEVKKP | VFPLAPSS | PSSLSASV |
| GGGCxhOKT3 | SSDKTHT | GASVKVS | GS (SEQ | GASVKVS | KSTSGGT | GDRVTIT |
| | CPPCPAP | CKASGYT | ID NO: | CKASGYT | AALGCLV | CSASSSV |
| | EFEGGPS | FTNYYM | 725) | FTRYTMH | KDYFPEP | SYMNWY |
| | VFLFPPKP | HWVRQA | | WVRQAP | VTVSWNS | QQKPGK |
| | KDTLMIS | PGQGLE | | GQGLEW | GALTSGV | APKRLIY |
| | RTPEVTC | WMGMIN | | MGYINPS | HTFPAVL | DTSKLAS |
| | VVVDVS | PSGGGTS | | RGYTNY | QSSGLYS | GVPSRFS |
| | HEDPEVK | YAQKFQ | | NQKFKDR | LSSVVTV | GSGSGTD |
| | FNWYVD | GRVTMT | | VTLTTDK | PSSSLGT | FTLTISSL |
| | GVEVHN | RDTSTST | | SSSTAYM | QTYICNV | QPEDFAT |
| | AKTKPRE | VYMELSS | | ELSSLRSE | NHKPSNT | YYCQQW |
| | EQYNSTY | LRSEDTA | | DTAVYY | KVDKRV | SSNPFTFG |
| | RVVSVLT | VYYCAR | | CARYYD | EPKSCDK | QGTKLEI |
| | VLHQDW | GNPWELR | | DHYSLDY | THTCPPC | KRTVAAP |
| | LNGKEYK | LDYWGQ | | WGQGTL | PAPELLG | SVFIFPPS |
| | CKVSNKA | GTLVTVS | | VTVSS | GPSVFLFP | DEQLKSG |
| | LPASIEKT | SGGGGSG | | (SEQ ID | PKPKDTL | TASVVCL |
| | ISKAKGQ | GGGSDIQ | | NO: 126) | MISRTPE | LNNFYPR |
| | PREPQVY | MTQSPSS | | | VTCVVV | EAKVQW |
| | TLPPCRE | LSASVGD | | | DVSHEDP | KVDNAL |
| | EMTKNQ | RVTITCQ | | | EVKFNW | QSGNSQE |
| | VSLWCLV | ASQDISN | | | YVDGVE | SVTEQDS |
| | KGFYPSD | YLNWYQ | | | VHNAKT | KDSTYSL |
| | IAVEWES | QKPGKAP | | | KPREEQY | SSTLTLSK |
| | NGQPENN | KLLIYAA | | | NSTYRVV | ADYEKH |
| | YKTTPPV | SSLQSGV | | | SVLTVLH | KVYACE |
| | LDSDGSF | PSRFSGS | | | QDWLNG | VTHQGLS |
| | FLYSKLT | GSGTDFT | | | KEYKCK | SPVTKSF |
| | VDKSRW | LTISSLQP | | | VSNKALP | NRGEC |
| | QQGNVFS | EDFATYY | | | APIEKTIS | (SEQ ID |
| | CSVMHE | CQQYYSY | | | KAKGQPR | NO: 712) |
| | ALHNHYT | PFTFGPG | | | EPQVCTL | |
| | QKSLSLS | TKVDIK | | | PPSREEM | |
| | PGK (SEQ | (SEQ ID | | | TKNQVSL | |
| | ID NO: | NO: 721) | | | SCAVKGF | |
| | 724) | | | | YPSDIAV | |
| | | | | | EWESNG | |
| | | | | | QPENNYK | |
| | | | | | (Chain2) | |
| | | | | | TTPPVLD | |
| | | | | | SDGSFFL | |
| | | | | | VSKLTVD | |
| | | | | | KSRWQQ | |
| | | | | | GNVFSCS | |
| | | | | | VMHEAL | |
| | | | | | HNHYTQ | |
| | | | | | KSLSLSP | |
| | | | | | GK (SEQ | |
| | | | | | ID NO: | |
| | | | | | 722) | |

TABLE 46

| | | | | | CH1- | |
|---|---|---|---|---|---|---|
| | | ABR | | | hinge- | |
| | Linker-Fc | (Chains 1 | Linker | VH | CH2-CH3 | LC |
| Name | (Chain1) | and 2) | (Chain2) | (Chain2) | (Chain2) | (Chain 3) |
| D-1H11- | GGGCGSE | QVQLVQS | GGGCGG | QVQLVQS | ASTKGPS | DIQMTQS |
| LS- | PKSSDKT | GAEVKKP | GGSGSGG | GAEVKKP | VFPLAPSS | PSSLSASV |
| GGCxhOKT3 | HTCPPCP | GASVKVS | GGS (SEQ | GASVKVS | KSTSGGT | GDRVTIT |
| | APEFEGG | CKASGYT | ID NO: | CKASGYT | AALGCLV | CSASSSV |
| | PSVFLFPP | FTNYYM | 411) | FTRYTMH | KDYFPEP | SYMNWY |
| | KPKDTLM | HWVRQA | | WVRQAP | VTVSWNS | QQKPGK |
| | ISRTPEVT | PGQGLE | | GQGLEW | GALTSGV | APKRLIY |
| | CVVVDVS | WMGMIN | | MGYINPS | HTFPAVL | DTSKLAS |
| | HEDPEVK | PSGGGTS | | RGYTNY | QSSGLYS | GVPSRFS |
| | FNWYVD | YAQKFQ | | NQKFKDR | LSSVVTV | GSGSGTD |
| | GVEVHN | GRVTMT | | VTLTTDK | PSSSLGT | FTLTISSL |
| | AKTKPRE | RDTSTST | | SSSTAYM | QTYICNV | QPEDFAT |
| | EQYNSTY | VYMELSS | | ELSSLRSE | NHKPSNT | YYCQQW |
| | RVVSVLT | LRSEDTA | | DTAVYY | KVDKRV | SSNPFTFG |
| | VLHQDW | VYYCAR | | CARYYD | EPKSCDK | QGTKLEI |
| | LNGKEYK | GNPWELR | | DHYSLDY | THTCPPC | KRTVAAP |
| | CKVSNKA | LDYWGQ | | WGQGTL | PAPELLG | SVFIFPPS |
| | LPASIEKT | GTLVTVS | | VTVSS | GPSVFLFP | DEQLKSG |
| | ISKAKGQ | SGGGGSG | | (SEQ ID | PKPKDTL | TASVVCL |
| | PREPQVY | GGGSDIQ | | NO: 126) | MISRTPE | LNNFYPR |
| | TLPPCRE | MTQSPSS | | | VTCVVV | EAKVQW |
| | EMTKNQ | LSASVGD | | | DVSHEDP | KVDNAL |
| | VSLWCLV | RVTITCQ | | | EVKFNW | QSGNSQE |
| | KGFYPSD | ASQDISN | | | YVDGVE | SVTEQDS |
| | IAVEWES | YLNWYQ | | | VHNAKT | KDSTYSL |
| | NGQPENN | QKPGKAP | | | KPREEQY | SSTLTLSK |
| | YKTTPPV | KLLIYAA | | | NSTYRVV | ADYEKH |
| | LDSDGSF | SSLQSGV | | | SVLTVLH | KVYACE |
| | FLYSKLT | PSRFSGS | | | QDWLNG | VTHQGLS |
| | VDKSRW | GSGTDFT | | | KEYKCK | SPVTKSF |
| | QQGNVFS | LTISSLQP | | | VSNKALP | NRGEC |
| | CSVMHE | EDFATYY | | | APIEKTIS | (SEQ ID |
| | ALHNHYT | CQQYYSY | | | KAKGQPR | NO: 712) |
| | QKSLSLS | PFTFGPG | | | EPQVCTL | |
| | PGK (SEQ | TKVDIK | | | PPSREEM | |
| | ID NO: | (SEQ ID | | | TKNQVSL | |
| | 726) | NO: 721) | | | SCAVKGF | |
| | | | | | YPSDIAV | |
| | | | | | EWESNG | |
| | | | | | QPENNYK | |
| | | | | | TTPPVLD | |
| | | | | | SDGSFFL | |
| | | | | | VSKLTVD | |
| | | | | | KSRWQQ | |
| | | | | | GNVFSCS | |
| | | | | | VMHEAL | |
| | | | | | HNHYTQ | |
| | | | | | KSLSLSP | |
| | | | | | GK (SEQ | |
| | | | | | ID NO: | |
| | | | | | 722) | |

TABLE 47

| | | | | | CH1- | |
|---|---|---|---|---|---|---|
| | | ABR | | | hinge- | |
| | Linker-Fc | (Chains 1 | Linker | VH | CH2-CH3 | LC |
| Name | (Chain1) | and 2) | (Chain2) | (Chain2) | (Chain2) | (Chain 3) |
| D-1H11- | GGGCGG | QVQLVQS | GGGCGS | QVQLVQS | ASTKGPS | DIQMTQS |
| SL- | GGSGSEP | GAEVKKP | (SEQ ID | GAEVKKP | VFPLAPSS | PSSLSASV |
| GGCxhOKT3 | KSSDKTH | GASVKVS | NO: 413) | GASVKVS | KSTSGGT | GDRVTIT |
| | TCPPCPA | CKASGYT | | CKASGYT | AALGCLV | CSASSSV |
| | PEFEGGP | FTNYYM | | FTRYTMH | KDYFPEP | SYMNWY |
| | SVFLFPPK | HWVRQA | | WVRQAP | VTVSWNS | QQKPGK |
| | PKDTLMI | PGQGLE | | GQGLEW | GALTSGV | APKRLIY |
| | SRTPEVT | WMGMIN | | MGYINPS | HTFPAVL | DTSKLAS |
| | CVVVDVS | PSGGGTS | | RGYTNY | QSSGLYS | GVPSRFS |
| | HEDPEVK | YAQKFQ | | NQKFKDR | LSSVVTV | GSGSGTD |
| | FNWYVD | GRVTMT | | VTLTTDK | PSSSLGT | FTLTISSL |
| | GVEVHN | RDTSTST | | SSSTAYM | QTYICNV | QPEDFAT |
| | AKTKPRE | VYMELSS | | ELSSLRSE | NHKPSNT | YYCQQW |
| | EQYNSTY | LRSEDTA | | DTAVYY | KVDKRV | SSNPFTFG |
| | RVVSVLT | VYYCAR | | CARYYD | EPKSCDK | QGTKLEI |
| | VLHQDW | GNPWELR | | DHYSLDY | THTCPPC | KRTVAAP |
| | LNGKEYK | LDYWGQ | | WGQGTL | PAPELLG | SVFIFPPS |
| | CKVSNKA | GTLVTVS | | VTVSS | GPSVFLFP | DEQLKSG |
| | LPASIEKT | SGGGGSG | | (SEQ ID | PKPKDTL | TASVVCL |
| | ISKAKGQ | GGGSDIQ | | NO: 126) | MISRTPE | LNNFYPR |
| | PREPQVY | MTQSPSS | | | VTCVVV | EAKVQW |
| | TLPPCRE | LSASVGD | | | DVSHEDP | KVDNAL |
| | EMTKNQ | RVTITCQ | | | EVKFNW | QSGNSQE |
| | VSLWCLV | ASQDISN | | | YVDGVE | SVTEQDS |
| | KGFYPSD | YLNWYQ | | | VHNAKT | KDSTYSL |
| | IAVEWES | QKPGKAP | | | KPREEQY | SSTLTLSK |
| | NGQPENN | KLLIYAA | | | NSTYRVV | ADYEKH |
| | YKTTPPV | SSLQSGV | | | SVLTVLH | KVYACE |
| | LDSDGSF | PSRFSGS | | | QDWLNG | VTHQGLS |
| | FLYSKLT | GSGTDFT | | | KEYKCK | SPVTKSF |
| | VDKSRW | LTISSLQP | | | VSNKALP | NRGEC |
| | QQGNVFS | EDFATYY | | | APIEKTIS | (SEQ ID |
| | CSVMHE | CQQYYSY | | | KAKGQPR | NO: 712) |
| | ALHNHYT | PFTFGPG | | | EPQVCTL | |
| | QKSLSLS | TKVDIK | | | PPSREEM | |
| | PGK (SEQ | (SEQ ID | | | TKNQVSL | |
| | ID NO: | NO: 721) | | | SCAVKGF | |
| | 727) | | | | YPSDIAV | |
| | | | | | EWESNG | |
| | | | | | QPENNYK | |
| | | | | | TTPPVLD | |
| | | | | | SDGSFFL | |
| | | | | | VSKLTVD | |
| | | | | | KSRWQQ | |
| | | | | | GNVFSCS | |
| | | | | | VMHEAL | |
| | | | | | HNHYTQ | |
| | | | | | KSLSLSP | |
| | | | | | GK (SEQ | |
| | | | | | ID NO: | |
| | | | | | 722) | |

TABLE 48

| | | VH-CH1- | | |
|---|---|---|---|---|
| | ABR-Linker- | hinge-CH2- | LC | ABR |
| Name | Fc (Chain1) | CH3 (Chain2) | (Chain 3) | (Chain 4) |
| D2-1H11-L- | GGCGGGGSGS | QVQLVQSGAE | DIQMTQSPSSL | QVQLVQSGAE |
| GGCxhOKT3 | EPKSSDKTHTC | VKKPGASVKV | SASVGDRVTIT | VKKPGASVKV |
| | PPCPAPEFEGG | SCKASGYTFTR | CSASSSVSYM | SCKASGYTFTN |
| | PSVFLFPPKPK | YTMHWVRQA | NWYQQKPGK | YYMHWVRQA |
| | DTLMISRTPEV | PGQGLEWMG | APKRLIYDTSK | PGQGLEWMG |
| | TCVVVDVSHE | YINPSRGYTNY | LASGVPSRFSG | MINPSGGGTSY |
| | DPEVKFNWYV | NQKFKDRVTL | SGSGTDFTLTIS | AQKFQGRVTM |
| | DGVEVHNAKT | TTDKSSSTAY | SLQPEDFATYY | TRDTSTSTVY |
| | KPREEQYNSTY | MELSSLRSEDT | CQQWSSNPFTF | MELSSLRSEDT |
| | RVVSVLTVLH | AVYYCARYYD | GQGTKLEIKRT | AVYYCARGNP |
| | QDWLNGKEYK | DHYSLDYWGQ | VAAPSVFIFPPS | WELRLDYWG |
| | CKVSNKALPA | GTLVTVSSAST | DEQLKSGTAS | QGTLVTVSSG |
| | SIEKTISKAKG | KGPSVFPLAPS | VVCLLNNFYP | GGGSGGGGSD |
| | QPREPQVYTLP | SKSTSGGTAAL | REAKVQWKV | IQMTQSPSSLS |
| | PCREEMTKNQ | GCLVKDYFPEP | DNALQSGNSQ | ASVGDRVTITC |
| | VSLWCLVKGF | VTVSWNSGAL | ESVTEQDSKDS | QASQDISNYLN |
| | YPSDIAVEWES | TSGVHTFPAVL | TYSLSSTLTLS | WYQQKPGKAP |
| | NGQPENNYKT | QSSGLYSLSSV | KADYEKHKVY | KLLIYAASSLQ |
| | TPPVLDSDGSF | VTVPSSSLGTQ | ACEVTHQGLSS | SGVPSRFSGSG |
| | FLYSKLTVDKS | TYICNVNHKPS | PVTKSFNRGEC | SGTDFTLTISSL |
| | RWQQGNVFSC | NTKVDKRVEP | (SEQ ID NO: | QPEDFATYYC |
| | SVMHEALHNH | KSCDKTHTCPP | 712) | QQYYSYPFTFG |
| | YTQKSLSLSPG | CPAPELLGGPS | | PGTKVDIKGG |
| | K (SEQ ID NO: | VFLFPPKPKDT | | C (SEQ ID NO: |
| | 720) | LMISRTPEVTC | | 193) |
| | | VVVDVSHEDP | | |
| | | EVKFNWYVDG | | |
| | | VEVHNAKTKP | | |
| | | REEQYNSTYR | | |
| | | VVSVLTVLHQ | | |
| | | DWLNGKEYKC | | |
| | | KVSNKALPAPI | | |
| | | EKTISKAKGQP | | |
| | | REPQVCTLPPS | | |
| | | REEMTKNQVS | | |
| | | LSCAVKGFYPS | | |
| | | DIAVEWESNG | | |
| | | QPENNYKTTPP | | |
| | | VLDSDGSFFLV | | |
| | | SKLTVDKSRW | | |
| | | QQGNVFSCSV | | |
| | | MHEALHNHYT | | |
| | | QKSLSLSPGK | | |
| | | (SEQ ID NO: | | |
| | | 728) | | |

TABLE 49

| | | VH-CH1- | | |
|---|---|---|---|---|
| | ABR-Linker- | hinge-CH2- | LC | ABR |
| | Fc (Chain1) | CH3 (Chain2) | (Chain 3) | (Chain 4) |
| D2-1NameH11- | GGCGSEPKSSD | QVQLVQSGAE | DIQMTQSPSSL | QVQLVQSGAE |
| GGCxhOKT3 | KTHTCPPCPAP | VKKPGASVKV | SASVGDRVTIT | VKKPGASVKV |
| | EFEGGPSVFLF | SCKASGYTFTR | CSASSSVSYM | SCKASGYTFTN |
| | PPKPKDTLMIS | YTMHWVRQA | NWYQQKPGK | YYMHWVRQA |
| | RTPEVTCVVV | PGQGLEWMG | APKRLIYDTSK | PGQGLEWMG |
| | DVSHEDPEVK | YINPSRGYTNY | LASGVPSRFSG | MINPSGGGTSY |
| | FNWYVDGVEV | NQKFKDRVTL | SGSGTDFTLTIS | AQKFQGRVTM |
| | HNAKTKPREE | TTDKSSSTAY | SLQPEDFATYY | TRDTSTSTVY |
| | QYNSTYRVVS | MELSSLRSEDT | CQQWSSNPFTF | MELSSLRSEDT |
| | VLTVLHQDWL | AVYYCARYYD | GQGTKLEIKRT | AVYYCARGNP |
| | NGKEYKCKVS | DHYSLDYWGQ | VAAPSVFIFPPS | WELRLDYWG |
| | NKALPASIEKTI | GTLVTVSSAST | DEQLKSGTAS | QGTLVTVSSG |
| | SKAKGQPREP | KGPSVFPLAPS | VVCLLNNFYP | GGGSGGGGSD |
| | QVYTLPPCREE | SKSTSGGTAAL | REAKVQWKV | IQMTQSPSSLS |
| | MTKNQVSLWC | GCLVKDYFPEP | DNALQSGNSQ | ASVGDRVTITC |
| | LVKGFYPSDIA | VTVSWNSGAL | ESVTEQDSKDS | QASQDISNYLN |
| | VEWESNGQPE | TSGVHTFPAVL | TYSLSSTLTLS | WYQQKPGKAP |
| | NNYKTTPPVL | QSSGLYSLSSV | KADYEKHKVY | KLLIYAASSLQ |
| | DSDGSFFLYSK | VTVPSSSLGTQ | ACEVTHQGLSS | SGVPSRFSGSG |
| | LTVDKSRWQQ | TYICNVNHKPS | PVTKSFNRGEC | SGTDFTLTISSL |
| | GNVFSCSVMH | NTKVDKRVEP | (SEQ ID NO: | QPEDFATYYC |
| | EALHNHYTQK | KSCDKTHTCPP | 712) | QQYYSYPFTFG |
| | SLSLSPGK | CPAPELLGGPS | | PGTKVDIKGG |
| | (SEQ ID NO: | VFLFPPKPKDT | | C (SEQ ID NO: |
| | 723) | LMISRTPEVTC | | 193) |
| | | VVVDVSHEDP | | |
| | | EVKFNWYVDG | | |
| | | VEVHNAKTKP | | |
| | | REEQYNSTYR | | |
| | | VVSVLTVLHQ | | |
| | | DWLNGKEYKC | | |
| | | KVSNKALPAPI | | |
| | | EKTISKAKGQP | | |
| | | REPQVCTLPPS | | |
| | | REEMTKNQVS | | |
| | | LSCAVKGFYPS | | |
| | | DIAVEWESNG | | |
| | | QPENNYKTTPP | | |
| | | VLDSDGSFFLV | | |
| | | SKLTVDKSRW | | |
| | | QQGNVFSCSV | | |
| | | MHEALHNHYT | | |
| | | QKSLSLSPGK | | |
| | | (SEQ ID NO: | | |
| | | 728) | | |

TABLE 50

| | | VH-CH1- | | |
|---|---|---|---|---|
| | ABR-Linker- | hinge-CH2- | LC | ABR |
| Name | Fc (Chain1) | CH3 (Chain2) | (Chain 3) | (Chain 4) |
| D2-1H11-L- | GGGCGGGSGS | QVQLVQSGAE | DIQMTQSPSSL | QVQLVQSGAE |
| GGGCxhOKT3 | EPKSSDKTHTC | VKKPGASVKV | SASVGDRVTIT | VKKPGASVKV |
| | PPCPAPEFEGG | SCKASGYTFTR | CSASSSVSYM | SCKASGYTFTN |
| | PSVFLFPPKPK | YTMHWVRQA | NWYQQKPGK | YYMHWVRQA |
| | DTLMISRTPEV | PGQGLEWMG | APKRLIYDTSK | PGQGLEWMG |
| | TCVVVDVSHE | YINPSRGYTNY | LASGVPSRFSG | MINPSGGGTSY |
| | DPEVKFNWYV | NQKFKDRVTL | SGSGTDFTLTIS | AQKFQGRVTM |
| | DGVEVHNAKT | TTDKSSSTAY | SLQPEDFATYY | TRDTSTSTVY |
| | KPREEQYNSTY | MELSSLRSEDT | CQQWSSNPFTF | MELSSLRSEDT |
| | RVVSVLTVLH | AVYYCARYYD | GQGTKLEIKRT | AVYYCARGNP |
| | QDWLNGKEYK | DHYSLDYWGQ | VAAPSVFIFPPS | WELRLDYWG |
| | CKVSNKALPA | GTLVTVSSAST | DEQLKSGTAS | QGTLVTVSSG |
| | SIEKTISKAKG | KGPSVFPLAPS | VVCLLNNFYP | GGGSGGGGSD |
| | QPREPQVYTLP | SKSTSGGTAAL | REAKVQWKV | IQMTQSPSSLS |
| | PCREEMTKNQ | GCLVKDYFPEP | DNALQSGNSQ | ASVGDRVTITC |
| | VSLWCLVKGF | VTVSWNSGAL | ESVTEQDSKDS | QASQDISNYLN |
| | YPSDIAVEWES | TSGVHTFPAVL | TYSLSSTLTLS | WYQQKPGKAP |
| | NGQPENNYKT | QSSGLYSLSSV | KADYEKHKVY | KLLIYAASSLQ |
| | TPPVLDSDGSF | VTVPSSSLGTQ | ACEVTHQGLSS | SGVPSRFSGSG |
| | FLYSKLTVDKS | TYICNVNHKPS | PVTKSFNRGEC | SGTDFTLTISSL |
| | RWQQGNVFSC | NTKVDKRVEP | (SEQ ID NO: | QPEDFATYYC |
| | SVMHEALHNH | KSCDKTHTCPP | 712) | QQYYSYPFTFG |
| | YTQKSLSLSPG | CPAPELLGGPS | | PGTKVDIKGG |
| | K (SEQ ID NO: | VFLFPPKPKDT | | GC (SEQ ID NO: |
| | 724) | LMISRTPEVTC | | 418) |
| | | VVVDVSHEDP | | |
| | | EVKFNWYVDG | | |
| | | VEVHNAKTKP | | |
| | | REEQYNSTYR | | |
| | | VVSVLTVLHQ | | |
| | | DWLNGKEYKC | | |
| | | KVSNKALPAPI | | |
| | | EKTISKAKGQP | | |
| | | REPQVCTLPPS | | |
| | | REEMTKNQVS | | |
| | | LSCAVKGFYPS | | |
| | | DIAVEWESNG | | |
| | | QPENNYKTTPP | | |
| | | VLDSDGSFFLV | | |
| | | SKLTVDKSRW | | |
| | | QQGNVFSCSV | | |
| | | MHEALHNHYT | | |
| | | QKSLSLSPGK | | |
| | | (SEQ ID NO: | | |
| | | 728) | | |

TABLE 51

| | | VH-CH1- | | |
|---|---|---|---|---|
| | ABR-Linker- | hinge-CH2- | LC | ABR |
| Name | Fc (Chain1) | CH3 (Chain2) | (Chain 3) | (Chain 4) |
| D2-1H11- | GGGCGSEPKSS | QVQLVQSGAE | DIQMTQSPSSL | QVQLVQSGAE |
| GGGCxhOKT3 | DKTHTCPPCPA | VKKPGASVKV | SASVGDRVTIT | VKKPGASVKV |
| | PEFEGGPSVFL | SCKASGYTFTR | CSASSSVSYM | SCKASGYTFTN |
| | FPPKPKDTLMI | YTMHWVRQA | NWYQQKPGK | YYMHWVRQA |
| | SRTPEVTCVVV | PGQGLEWMG | APKRLIYDTSK | PGQGLEWMG |
| | DVSHEDPEVK | YINPSRGYTNY | LASGVPSRFSG | MINPSGGGTSY |
| | FNWYVDGVEV | NQKFKDRVTL | SGSGTDFTLTIS | AQKFQGRVTM |
| | HNAKTKPREE | TTDKSSSTAY | SLQPEDFATYY | TRDTSTSTVY |
| | QYNSTYRVVS | MELSSLRSEDT | CQQWSSNPFTF | MELSSLRSEDT |
| | VLTVLHQDWL | AVYYCARYYD | GQGTKLEIKRT | AVYYCARGNP |
| | NGKEYKCKVS | DHYSLDYWGQ | VAAPSVFIFPPS | WELRLDYWG |
| | NKALPASIEKTI | GTLVTVSSAST | DEQLKSGTAS | QGTLVTVSSG |
| | SKAKGQPREP | KGPSVFPLAPS | VVCLLNNFYP | GGGSGGGGSD |
| | QVYTLPPCREE | SKSTSGGTAAL | REAKVQWKV | IQMTQSPSSLS |
| | MTKNQVSLWC | GCLVKDYFPEP | DNALQSGNSQ | ASVGDRVTITC |
| | LVKGFYPSDIA | VTVSWNSGAL | ESVTEQDSKDS | QASQDISNYLN |
| | VEWESNGQPE | TSGVHTFPAVL | TYSLSSTLTLS | WYQQKPGKAP |
| | NNYKTTPPVL | QSSGLYSLSSV | KADYEKHKVY | KLLIYAASSLQ |
| | DSDGSFFLYSK | VTVPSSSLGTQ | ACEVTHQGLSS | SGVPSRFSGSG |
| | LTVDKSRWQQ | TYICNVNHKPS | PVTKSFNRGEC | SGTDFTLTISSL |
| | GNVFSCSVMH | NTKVDKRVEP | (SEQ ID NO: | QPEDFATYYC |
| | EALHNHYTQK | KSCDKTHTCPP | 712) | QQYYSYPFTFG |
| | SLSLSPGK | CPAPELLGGPS | | PGTKVDIKGG |
| | (SEQ ID NO: | VFLFPPKPKDT | | GC (SEQ ID NO: |
| | 726) | LMISRTPEVTC | | 418) |
| | | VVVDVSHEDP | | |
| | | EVKFNWYVDG | | |
| | | VEVHNAKTKP | | |
| | | REEQYNSTYR | | |
| | | VVSVLTVLHQ | | |
| | | DWLNGKEYKC | | |
| | | KVSNKALPAPI | | |
| | | EKTISKAKGQP | | |
| | | REPQVCTLPPS | | |
| | | REEMTKNQVS | | |
| | | LSCAVKGFYPS | | |
| | | DIAVEWESNG | | |
| | | QPENNYKTTPP | | |
| | | VLDSDGSFFLV | | |
| | | SKLTVDKSRW | | |
| | | QQGNVFSCSV | | |
| | | MHEALHNHYT | | |
| | | QKSLSLSPGK | | |
| | | (SEQ ID NO: | | |
| | | 728) | | |

TABLE 54

| | | L4 Linker-VH- | | |
|---|---|---|---|---|
| | | CH1-hinge- | | ABR |
| | L3 Linker-Fc | CH2-CH3 | LC | (Chain 4; VH- |
| Name | (Chain1) | (Chain2) | (Chain 3) | Linker-VL) |
| 1B03-GGGGS- | GGGGSEPKSSD | GGGGSGGGGS | DIQMTQSPSSL | EVQLLESGGGL |
| UCHT1v9 | KTHTCPPCPAP | EVQLVESGGG | SASVGDRVTIT | VQPGGSLRLSC |
| | EFEGGPSVFLF | LVQPGGSLRL | CRASQDIRNYL | AASGFTFSDYY |
| | PPKPKDTLMIS | SCAASGYSFT | NWYQQKPGK | MSWVRQAPGK |
| | RTPEVTCVVV | GYTMNWVRQ | APKLLIYYTSR | GLEWVSGINWN |
| | DVSHEDPEVK | APGKGLEWV | LESGVPSRFSG | GGSTGYADSVK |
| | FNWYVDGVEV | ALINPYKGVS | SGSGTDYTLTI | GRFTISRDNSKN |
| | HNAKTKPREE | TYNQKFKDRF | SSLQPEDFATY | TLYLQMNSLRA |
| | QYNSTYRVVS | TISVDKSKNT | YCQQGNTLPW | EDTAVYYCARV |
| | VLTVLHQDWL | AYLQMNSLR | TFGQGTKVEIK | EQGYDIYYYYY |
| | NGKEYKCKVS | AEDTAVYYCA | RTVAAPSVFIF | MDVWGKGTTV |
| | NKALPASIEKTI | RSGYYGDSD | PPSDEQLKSGT | TVSSGGGGSGG |
| | SKAKGQPREP | WYFDVWGQG | ASVVCLLNNF | GGSDIQMTQS |
| | QVYTLPPCREE | TLVTVSSASTK | YPREAKVQWK | PSSLSASVGDR |
| | MTKNQVSLWC | GPSVFPLAPSSK | VDNALQSGNS | VTITCRASQSI |
| | LVKGFYPSDIA | STSGGTAALGC | QESVTEQDSK | SSYLNWYQQ |
| | VEWESNGQPE | LVKDYFPEPVT | DSTYSLSSTLT | KPGKAPKLLI |
| | NNYKTTPPVL | VSWNSGALTSG | LSKADYEKHK | YKASSLESGV |
| | DSDGSFFLYSK | VHTFPAVLQSS | VYACEVTHQG | PSRFSGSGSGT |
| | LTVDKSRWQQ | GLYSLSSVVTVP | LSSPVTKSFNR | DFTLTISSLQP |
| | GNVFSCSVMH | SSSLGTQTYICN | GEC | EDFATYYCQQ |
| | EALHNHYTQK | VNHKPSNTKVD | (SEQ ID NO: | SYSAPYTFGP |
| | SLSLSPGK | KRVepkscdkthtcp | 737) | GTKVDIK |
| | (SEQ ID NO: | [symbol] | | (SEQ ID NO: |
| | 735) | [symbol] | | 738) |
| | | [symbol] | | |
| | | [symbol] | | |
| | | [symbol] | | |
| | | [symbol] | | |
| | | [symbol] | | |
| | | [symbol] | | |
| | | [symbol] | | |
| | | [symbol] | | |
| | | [symbol] GQ | | |
| | | PREPQVCTLPP | | |
| | | SREEMTKNQVS | | |
| | | LSCAVKGFYPS | | |
| | | DIAVEWESNGQ | | |
| | | PENNYKTTPPV | | |
| | | LDSDGSFFLVS | | |
| | | KLTVDKSRWQQ | | |
| | | GNVFSCSVMHE | | |
| | | ALHNRFTQKSL | | |
| | | SLSPGK | | |
| | | (SEQ ID NO: | | |
| | | 736) | | |

Fragments of the Format 4 antibody herein have text formatted differently. In Chain 1, the L3 linker is non-bold and underlined, the Fc is non-bold. In Chain 2, the L4 linker is non-bold and underlined, the VH is bold, the CH1 is non-bold, italics and underlined, the hinge is lower case, CH2 is bold and italics, CH3 is non-bold and italics. In Chain 3, the CL is underlined. In Chain 4, the VH is non-bold and italics, the linker (L1 or L2) is non-bold and underlined, and the VL is bold.

TABLE 55

| | | L4 Linker-VH- | L3 Linker-Fc | L4 Linker-VH- |
|---|---|---|---|---|
| | | CH1-hinge- | (Chain1) | CH1-hinge- |
| | L3 Linker-Fc | CH2-CH3 | | CH2-CH3 |
| Name | (Chain1) | (Chain2) | | (Chain2) |
| 1B03- | GGGGSEPKSSD | GGGGSGGGGS | DIQMTQSPSSL | EVQLLESGGG |
| GGGGS- | KTHTCPPCPAP | QVQLVQSGAE | SASVGDRVTIT | LVQPGGSLRLS |
| xhOKT3a | EFEGGPSVFLF | VKKPGASVKV | CSASSSVSYM | CAASGFTFSDY |
| | PPKPKDTLMIS | SCKASGYTFTR | NWYQQKPGK | YMSWVRQAP |
| | RTPEVTCVVV | YTMHWVRQA | APKRLIYDTSK | GKGLEWVSGI |
| | DVSHEDPEVK | PGQGLEWMG | LASGVPSRFSG | NWNGGSTGYA |
| | FNWYVDGVEV | YINPSRGYTNY | SGSGTDFTLTIS | DSVKGRFTISR |
| | HNAKTKPREE | NQKFKDRVTL | SLQPEDFATYY | DNSKNTLYLQ |
| | QYNSTYRVVS | TTDKSSSTAY | CQQWSSNPFTF | MNSLRAEDTA |
| | VLTVLHQDWL | MELSSLRSEDT | GQGTKLEIKRT | VYYCARVEQG |
| | NGKEYKCKVS | AVYYCARYYD | VAAPSVFIFPPS | YDIYYYYYMD |
| | NKALPASIEKTI | DHYSLDYWGQ | DEQLKSGTAS | VWGKGTTVTV |
| | SKAKGQPREP | GTLVTVSSAST | VVCLLNNFYP | SSGGGGSGGG |
| | QVYTLPPCREE | KGPSVFPLAPS | REAKVQWKV | GSDIQMTQSPS |
| | MTKNQVSLWC | SKSTSGGTAAL | DNALQSGNSQ | SLSASVGDRVT |
| | LVKGFYPSDIA | GCLVKDYFPEP | ESVTEQDSKDS | ITCRASQSISSY |
| | VEWESNGQPE | VTVSWNSGAL | TYSLSSTLTLS | LNWYQQKPGK |
| | NNYKTTPPVL | TSGVHTFPAVL | KADYEKHKVY | APKLLIYKASS |
| | DSDGSFFLYSK | QSSGLYSLSSV | ACEVTHQGLSS | LESGVPSRFSG |
| | LTVDKSRWQQ | VTVPSSSLGTQ | PVTKSFNRGEC | SGSGTDFTLTIS |
| | GNVFSCSVMH | TYICNVNHKPS | (SEQ ID NO: | SLQPEDFATYY |
| | EALHNHYTQK | NTKVDKRVEP | 740) | CQQSYSAPYTF |
| | SLSLSPGK | KSCDKTHTCPP | | GPGTKVDIK |
| | (SEQ ID NO: | CPAPEFEGGPS | | (SEQ ID NO: |
| | 735) | VFLFPPKPKDT | | 738) |
| | | LMISRTPEVTC | | |
| | | VVVDVSHEDP | | |
| | | EVKFNWYVDG | | |
| | | VEVHNAKTKP | | |
| | | REEQYNSTYR | | |
| | | VVSVLTVLHQ | | |
| | | DWLNGKEYKC | | |
| | | KVSNKALPASI | | |
| | | EKTISKAKGQP | | |
| | | REPQVCTLPPS | | |
| | | REEMTKNQVS | | |
| | | LSCAVKGFYPS | | |
| | | DIAVEWESNG | | |
| | | QPENNYKTTPP | | |
| | | VLDSDGSFFLV | | |
| | | SKLTVDKSRW | | |
| | | QQGNVFSCSV | | |
| | | MHEALHNRFT | | |
| | | QKSLSLSPGK | | |
| | | (SEQ ID NO: | | |
| | | 739) | | |

1. An isolated multispecific antigen binding protein (ABP) comprising a first antigen binding region (ABR) and a second ABR that each specifically bind a first target antigen, a Fab that specifically binds an additional target antigen that is distinct from the first target antigen, and an Fc domain, wherein the ABP comprises a first polypeptide, a second polypeptide, and a third polypeptide, wherein the first polypeptide comprises, in an N→C direction, the first ABR-a first hinge-CH2-CH3, wherein the second polypeptide comprises, in an N→C direction, a VH domain of the Fab-a CH1 domain of the Fab-a second hinge-CH2-CH3, wherein the third polypeptide comprises, in an N→C direction, a VL domain of the Fab-a CL domain of the Fab;
wherein the second ABR is attached, directly or indirectly, to the N-terminus of the second polypeptide or the third polypeptide;
wherein the first ABR and second ABR each comprise, in an N→C direction: (i) a VH domain-a VL domain or (ii) a VL domain-VH domain;
wherein the VH domain of the first ABR is attached to the VL domain of the first ABR via a first linker;
wherein the VH domain of the second ABR is attached to the VL domain of the second ABR via a second linker;
wherein the first linker and second linker are each equal to or less than 20 amino acids in length.

2. The isolated multispecific ABP of claim 1, wherein the second ABR is attached, directly or indirectly, to the N-terminus of the second polypeptide,
wherein the first target antigen is an HLA-PEPTIDE target, wherein the HLA-PEPTIDE target comprises an HLA-restricted peptide complexed with an HLA Class I molecule,
wherein the additional target antigen is CD3,
wherein the first and second linker are each 5-10 amino acids in length,
wherein the CH2-CH3 of the first polypeptide and the CH2-CH3 of the second polypeptide comprise a variant CH2-CH3 domain,
wherein the variant CH2-CH3 domains comprises the amino acid substitutions of L234F, L235E, and P331S, according to the EU numbering system,
further comprising an S354C and T366W mutation in one variant CH2-CH3 domain and a Y349C, T366S, L368A and Y407V mutation in the other variant CH2-CH3 domain, according to EU numbering,
wherein the first hinge comprises a C220S mutation, according to EU numbering, and
wherein the CH2-CH3 of either the first polypeptide or the second polypeptide comprises a H435R_Y436F mutation, according to EU numbering.

3. The isolated multispecific ABP of claim 1, wherein the first linker and second linker are each less than 20 amino acids in length, optionally wherein the first linker and the second linker each have a length of 10, 8, or 5 amino acids or less, or wherein the first linker and second linker each consists of (GGGGS)₂ (SEQ ID NO: 4) or (GGGGS)_{N} (SEQ ID NO: 1), wherein _{N}=1-3, optionally wherein _{N}=².

4.-14. (canceled)

15. The isolated multispecific ABP of claim 1, wherein the second ABR is attached, directly or indirectly, to the N-terminus of the second polypeptide or to the N-terminus of the third polypeptide.

16.-22. (canceled)

23. The isolated multispecific ABP of claim 1, wherein the VL domain of the first ABR interacts with the VH domain of the second ABR and/or wherein the VH domain of the first ABR interacts with the VL domain of the second ABR.

24. The isolated multispecific ABP of claim 23, wherein the interaction of the VL domain of the first ABR with the VH domain of the second ABR and the interaction of the VH domain of the first ABR with the VL domain of the second ABR results in a circularized conformation.

25.-27. (canceled)

28. The isolated multispecific ABP of claim 1, wherein the VH domain of the first ABR comprises a cysteine at amino acid residue 44 of the VH domain according to the Kabat numbering system and wherein the VL domain of the first ABR comprises a cysteine residue at amino acid residue 100 of the VL domain according to the Kabat numbering system; and/or wherein the VH domain of the second ABR comprises a cysteine at amino acid residue 44 of the VH domain according to the Kabat numbering system and wherein the VL domain of the second ABR comprises a cysteine residue at amino acid residue 100 of the VL domain according to the Kabat numbering system.

29. (canceled)

30. (canceled)

31. The isolated multispecific ABP of claim 1, wherein the first ABR and second ABR bind to an HLA-peptide target with a dissociation constant (K_{D}) less than or equal to 100 nM, as measured by biolayer interferometry (BLI).

32.-43. (canceled)

44. An isolated multispecific antigen binding protein (ABP) comprising a first antigen binding region (ABR) and a second ABR that each specifically bind a first target antigen, a Fab that specifically binds an additional target antigen that is distinct from the first target antigen, and an Fc domain, wherein the ABP comprises a first polypeptide, a second polypeptide, and a third polypeptide, wherein the first polypeptide comprises, in an N→C direction, the first ABR-a first hinge-CH2-CH3, wherein the second polypeptide comprises, in an N→C direction, a VH domain of the Fab-a CH1 domain of the Fab-a second hinge-CH2-CH3, wherein the third polypeptide comprises, in an N→C direction, a VL domain of the Fab-a CL domain of the Fab;
wherein the second ABR is attached, directly or indirectly, to the N-terminus of the second polypeptide or the third polypeptide;
wherein the first ABR and second ABR each comprise, in an N→C direction: (i) a VH domain-a VL domain or (ii) a VL domain-VH domain;
wherein the VH domain of the first ABR is attached to the VL domain of the first ABR via a first linker; wherein the VH domain of the second ABR is attached to the VL domain of the second ABR via a second linker;
wherein the first linker and the second linker each comprise 14 amino acids;
wherein (i) the VH domain of the first ABR comprises a cysteine at amino acid residue 44 of the VH domain according to the Kabat numbering system and wherein the VL domain of the first ABR comprises a cysteine residue at amino acid residue 100 of the VL domain according to the Kabat numbering system or (ii) wherein the VH domain of the second ABR comprises a cysteine at amino acid residue 44 of the VH domain according to the Kabat numbering system and wherein the VL domain of the second ABR comprises a cysteine residue at amino acid residue 100 of the VL domain according to the Kabat numbering system.

45.-69. (canceled)

70. The isolated multispecific ABP of claim 1, wherein the Fab, the first ABR, and/or the second ABR bind to an HLA-PEPTIDE target, wherein the HLA-PEPTIDE target comprises an HLA-restricted peptide complexed with an HLA Class I molecule, wherein the HLA-restricted peptide is located in the peptide binding groove of an α1/α2 portion of the HLA Class I molecule, and wherein the HLA-PEPTIDE target is selected from Table A, Table A1, or Table A2.

71. (canceled)

72. The isolated multispecific ABP of claim 70, wherein either the first ABR or the second ABR binds to an additional target antigen, optionally wherein the additional target antigen is not an HLA-Peptide target.

73. (canceled)

74. (canceled)

75. The isolated multispecific ABP of claim 70, wherein
a. the HLA Class I molecule is HLA subtype A*01:01 and the HLA-restricted peptide comprises the sequence NTDNNLAVY (SEQ ID NO: 5),
b. the HLA Class I molecule is HLA subtype A*02:01 and the HLA-restricted peptide comprises the sequence AIFPGAVPAA (SEQ ID NO: 6);
c. the HLA Class I molecule is HLA subtype A*01:01 and the HLA-restricted peptide comprises the sequence ASSLPTTMNY (SEQ ID NO: 7);
d. the HLA Class I molecule is HLA subtype A*02:01 and the HLA-restricted peptide comprises the sequence LLASSILCA (SEQ ID NO: 8); or
e. the HLA Class I molecule is HLA subtype B*35:01 and the HLA-restricted peptide comprises the sequence EVDPIGHVY (SEQ ID NO: 9).

76. (canceled)

77. (canceled)

78. The isolated multispecific ABP of claim 1, wherein the additional target antigen is a cell surface molecule present on an effector cell.

79. The isolated multispecific ABP of claim 78, wherein the effector cell is a T cell or an NK cell.

80. The isolated multispecific ABP of claim 79, wherein the cell surface molecule is CD3, optionally CD3F, or CD28 or CD16.

81. (canceled)

82. (canceled)

83. (canceled)

84. The isolated multispecific ABP of claim 44, further comprising an engineered disulfide bridge between the third linker and fourth linker.

85. (canceled)

86. (canceled)

87. The isolated multispecific ABP of claim 1, wherein the CH2-CH3 domains of the first polypeptide and/or the CH2-CH3 domains of the second polypeptide comprise a variant CH2-CH3 domain, optionally wherein a sequence comprising the CH2-CH3 domains of the first polypeptide is distinct from a sequence comprising the CH2-CH3 domains of the second polypeptide.

88. (canceled)

89. (canceled)

90. The isolated multispecific ABP of claim 87, wherein i) the variant CH2-CH3 domain comprises a human IgG4 Fc region comprising one or more of the hinge stabilizing mutations S228P and L235E, or comprising one or more of the following mutations: E233P, F234V, and L235A, according to EU numbering, ii) the variant CH2-CH3 domain is a human IgG1 Fc region comprising one or more mutations to reduce Fc receptor binding, optionally wherein the one or more mutations are in residues selected from S228 (e.g., S228A), L234 (e.g., L234A), L235 (e.g., L235A), D265 (e.g., D265A), and N297 (e.g., N297A or N297Q), or optionally wherein the amino acid sequence ELLG (SEQ ID NO: 10), from amino acid position 233 to 236 of IgG1 or EFLG (SEQ ID NO: 11) of IgG4, is replaced by PVA, according to EU numbering, iii) the variant CH2-CH3 domain is a human IgG2 Fc region comprising one or more of mutations A330S and P331S, according to EU numbering, or iv) the variant CH2-CH3 domain comprises an amino acid substitution at one or more positions selected from 238, 265, 269, 270, 297, 327 and 329, optionally wherein the variant CH2-CH3 domain comprises substitutions at two or more of amino acid positions 265, 269, 270, 297 and 327, optionally wherein the variant CH2-CH3 domain comprises substitution of residues 265 or 297 with alanine, optionally wherein the variant CH2-CH3 domain comprises substitution of residues 265 and 297 with alanine, according to EU numbering.

91.-102. (canceled)

103. The isolated multispecific ABP of claim 87, wherein the variant CH2-CH3 domain of the first polypeptide comprises a knob-in-hole modification and/or the variant CH2-CH3 domain of the second polypeptide comprises a knob-in-hole modification.

104. (canceled)

105. (canceled)

106. The isolated multispecific ABP of claim 103, further comprising an engineered disulfide bridge between the CH2-CH3 domains, optionally wherein
a. the engineered disulfide bridge comprises a K392C mutation in one CH2-CH3 domain of the multispecific ABP, and a D399C in the other CH2-CH3 domain of the multispecific ABP, according to EU numbering,
b. the engineered disulfide bridge comprises a S354C mutation in one CH2-CH3 domain of the multispecific ABP, and a Y349C mutation in the other CH2-CH3 domain of the multispecific ABP, according to EU numbering, or
c. the engineered disulfide bridge comprises a 447C mutation in both CH2-CH3 domains of the multispecific ABP, which 447C mutations are provided by extension of the C-terminus of a CH3 domain incorporating a KSC tripeptide sequence, according to EU numbering.

107.-110. (canceled)

111. The isolated multispecific ABP of claim 87, wherein:
a. one of the variant CH2-CH3 domains comprises a F405A and a Y407V mutation and the other variant CH2-CH3 domain comprises a T394W mutation,
b. one of the variant CH2-CH3 domains comprises a F405A and a Y407V mutation and the other variant CH2-CH3 domain comprises a T366I and a T394W mutation,
c. a one of the variant CH2-CH3 domains comprises a F405A and a Y407V mutation and the other variant CH2-CH3 domain comprises a T366L and a T394W mutation,
d. one of the variant CH2-CH3 domains comprises a F405A and a Y407V mutation and the other variant CH2-CH3 domain comprises a T366L mutation, a K392M mutation, and a T394W mutation,
e. one of the variant CH2-CH3 domains comprises a L351Y mutation, a F405A mutation, and a Y407V mutation and the other variant CH2-CH3 domain comprises a T366L mutation, a K392M mutation, and a T394W mutation,
f. one of the variant CH2-CH3 domains comprises a T350V mutation, a L351Y mutation, a F405A mutation, and a Y407V mutation and the other variant CH2-CH3 domain comprises a T350V mutation, a T366L mutation, a K392M mutation, and a T394W mutation, or
g. one of the variant CH2-CH3 domains comprises a T350V mutation, a L351Y mutation, a F405A mutation, and a Y407V mutation and the other variant CH2-CH3 domain comprises a T350V mutation, a T366L mutation, a K392M mutation, and a T394W mutation, or
h. the variant CH2-CH3 domain is an IgG1 Fc comprising a K409R mutation in one CH2-CH3 domain and a mutation selected from a Y407, L368, F405, K370, and D399 mutation in the CH2-CH3 domain, or
i. the variant CH2-CH3 domain comprises a K409D and a K392D mutation in one CH2-CH3 domain, and a D399K and a E356K mutation in the other CH2-CH3 domain, according to EU numbering, or
j. the variant CH2-CH3 domain comprises a K409R mutation in one CH2-CH3 domain and a L368E or L368D mutation in the other CH2-CH3 domain, according to EU numbering, or
k. the variant CH2-CH3 domain comprises a K409R mutation in one CH2-CH3 domain and a L368E or L368D mutation in the other CH2-CH3 domain, according to EU numbering, or
l. the variant CH2-CH3 domain comprises a D221E, P228E, and L368E mutation in one the variant CH2-CH3 domain and a D221R, P228R, and K409R in the other CH2-CH3 domain, or
m. the variant CH2-CH3 domain comprises an S364H and F405A mutation in one CH2-CH3 domain and a Y349T and T394F mutation in the other CH2-CH3 domain, or
n. the variant CH2-CH3 domain comprises an E375Q and S364K mutation in one CH2-CH3 domain and a L368D and K370S mutation in the other CH2-CH3 domain, according to EU numbering, according to EU numbering.

112.-196. (canceled)

197. An engineered cell expressing a receptor comprising the multispecific ABP of claim 1.

198.-203. (canceled)

204. A pharmaceutical composition comprising the multispecific ABP of claim 1 and a pharmaceutically acceptable excipient.

205. A method of treating cancer in a subject, comprising administering to the subject an effective amount of the multispecific ABP of claim 1, optionally wherein the cancer is selected from a solid tumor and a hematological tumor.

206. (canceled)

207. A method of treating chronic viral disease in a subject, comprising administering to the subject an effective amount of the multispecific ABP of claim 1.

208.-211. (canceled)

212. A method of isolating a multispecific antibody, comprising:
(a) providing (i) a mixture that comprises an ABP comprising a light chain Kappa constant domain, optionally wherein the ABP is selected from any one of the preceding claims, and (ii) an anti-Kappa resin, wherein the anti-Kappa resin comprises a ligand having high specificity for a light chain Kappa constant domain, and wherein contaminants lacking a light chain Kappa constant domain do not bind the anti-Kappa resin;
(b) contacting (i) and (ii) under conditions that allow for differential binding to the anti-Kappa resin as compared to at least one contaminant, in the mixture, that lacks a light chain Kappa constant domain or has a different number of light chain Kappa constant domains relative to the ABP; and
(c) eluting the ABP from the anti-Kappa resin under conditions that allow for differential detachment of the ABP relative to the contaminant.

213.-257. (canceled)


SEQUENCE LISTING (740 sequences)

SEQ ID NO: 1 - protein, 15 aa, Artificial Sequence
  Description of Artificial Sequence Synthetic peptide
  SITE (1)..(15): This sequence may encompass 1-3 “Gly Gly Gly Gly Ser” repeating units
GGGGSGGGGSGGGGS

SEQ ID NO: 2 - protein, 12 aa, Homo sapiens
KSCDKTHTCPPC

SEQ ID NO: 3 - protein, 20 aa, Artificial Sequence
  Description of Artificial Sequence Synthetic peptide
GGGGSGGGGSGGGGSGGGGS

SEQ ID NO: 4 - protein, 10 aa, Artificial Sequence
  Description of Artificial Sequence Synthetic peptide
GGGGSGGGGS

SEQ ID NO: 5 - protein, 9 aa, Homo sapiens
NTDNNLAVY

SEQ ID NO: 6 - protein, 10 aa, Homo sapiens
AIFPGAVPAA

SEQ ID NO: 7 - protein, 10 aa, Homo sapiens
ASSLPTTMNY

SEQ ID NO: 8 - protein, 9 aa, Homo sapiens
LLASSILCA

SEQ ID NO: 9 - protein, 9 aa, Homo sapiens
EVDPIGHVY

SEQ ID NO: 10 - protein, 4 aa, Homo sapiens
ELLG

SEQ ID NO: 11 - protein, 4 aa, Homo sapiens
EFLG

SEQ ID NO: 12 - protein, 10 aa, Artificial Sequence
  Description of Artificial Sequence Synthetic peptide
CAATEWLGVW

SEQ ID NO: 13 - protein, 10 aa, Artificial Sequence
  Description of Artificial Sequence Synthetic peptide
CARANWLDYW

SEQ ID NO: 14 - protein, 10 aa, Artificial Sequence
  Description of Artificial Sequence Synthetic peptide
CARDWVLDYW

SEQ ID NO: 15 - protein, 10 aa, Artificial Sequence
  Description of Artificial Sequence Synthetic peptide
CARGEWLDYW

SEQ ID NO: 16 - protein, 10 aa, Artificial Sequence
  Description of Artificial Sequence Synthetic peptide
CARGWELGYW

SEQ ID NO: 17 - protein, 11 aa, Artificial Sequence
  Description of Artificial Sequence Synthetic peptide
CARDFVGYDDW

SEQ ID NO: 18 - protein, 11 aa, Artificial Sequence
  Description of Artificial Sequence Synthetic peptide
CARDYGDLDYW

SEQ ID NO: 19 - protein, 11 aa, Artificial Sequence [sequence omitted]

SEQ ID NO: 20 - protein, 12 aa, Artificial Sequence
  Description of Artificial Sequence Synthetic peptide
CARDGYSGLDVW

SEQ ID NO: 21 - protein, 12 aa, Artificial Sequence
  Description of Artificial Sequence Synthetic peptide
CARDSGVGMDVW

SEQ ID NO: 22 - protein, 13 aa, Artificial Sequence
  Description of Artificial Sequence Synthetic peptide
CARDGVAVASDYW

SEQ ID NO: 23 - protein, 13 aa, Artificial Sequence
  Description of Artificial Sequence Synthetic peptide
CARGVNVDDFDYW

SEQ ID NO: 24 - protein, 15 aa, Artificial Sequence
  Description of Artificial Sequence Synthetic peptide
CARGDYTGNWYFDLW

SEQ ID NO: 25 - protein, 17 aa, Artificial Sequence
  Description of Artificial Sequence Synthetic peptide
CARDQFYGGNSGGHDYW

SEQ ID NO: 26 - protein, 8 aa, Artificial Sequence
  Description of Artificial Sequence Synthetic peptide
CAREEDYW

SEQ ID NO: 27 - protein, 10 aa, Artificial Sequence [sequence omitted]

SEQ ID NO: 28 - protein, 10 aa, Artificial Sequence [sequence omitted]

SEQ ID NO: 29 - protein, 10 aa, Artificial Sequence [sequence omitted]

SEQ ID NO: 30 - protein, 12 aa, Artificial Sequence [sequence omitted]

SEQ ID NO: 31 - protein, 12 aa, Artificial Sequence
  Description of Artificial Sequence Synthetic peptide
CARDYGGYVDYW

SEQ ID NO: 32 - protein, 12 aa, Artificial Sequence
  Description of Artificial Sequence Synthetic peptide
CAREGPAALDVW

SEQ ID NO: 33 - protein, 12 aa, Artificial Sequence
  Description of Artificial Sequence Synthetic peptide
CARERRSGMDVW

SEQ ID NO: 34 - protein, 12 aa, Artificial Sequence
  Description of Artificial Sequence Synthetic peptide
CARVLQEGMDVW

SEQ ID NO: 35 - protein, 12 aa, Artificial Sequence
  Description of Artificial Sequence Synthetic peptide
CASERELPFDIW

SEQ ID NO: 36 - protein, 13 aa, Artificial Sequence
  Description of Artificial Sequence Synthetic peptide
CAKGGGGYGMDVW

SEQ ID NO: 37 - protein, 15 aa, Artificial Sequence
  Description of Artificial Sequence Synthetic peptide
CAAMGIAVAGGMDVW

SEQ ID NO: 38 - protein, 10 aa, Artificial Sequence [sequence omitted]

SEQ ID NO: 39 - protein, 11 aa, Artificial Sequence
  Description of Artificial Sequence Synthetic peptide
CATYDDGMDVW

SEQ ID NO: 40 - protein, 12 aa, Artificial Sequence
  Description of Artificial Sequence Synthetic peptide
CARGGGGALDYW

SEQ ID NO: 41 - protein, 13 aa, Artificial Sequence
  Description of Artificial Sequence Synthetic peptide
CALSGNYYGMDVW

SEQ ID NO: 42 - protein, 14 aa, Artificial Sequence
  Description of Artificial Sequence Synthetic peptide
CARGNPWELRLDYW

SEQ ID NO: 43 - protein, 13 aa, Artificial Sequence
  Description of Artificial Sequence Synthetic peptide
CARDKNYYGMDVW

SEQ ID NO: 44 - protein, 11 aa, Artificial Sequence [sequence omitted]

SEQ ID NO: 45 - protein, 11 aa, Artificial Sequence [sequence omitted]

SEQ ID NO: 46 - protein, 11 aa, Artificial Sequence [sequence omitted]

SEQ ID NO: 47 - protein, 11 aa, Artificial Sequence [sequence omitted]

SEQ ID NO: 48 - protein, 11 aa, Artificial Sequence
  Description of Artificial Sequence Synthetic peptide
CQQSYGVPYTF

SEQ ID NO: 49 - protein, 11 aa, Artificial Sequence [sequence omitted]

SEQ ID NO: 50 - protein, 11 aa, Artificial Sequence [sequence omitted]

SEQ ID NO: 51 - protein, 11 aa, Artificial Sequence [sequence omitted]

SEQ ID NO: 52 - protein, 11 aa, Artificial Sequence [sequence omitted]

SEQ ID NO: 53 - protein, 11 aa, Artificial Sequence [sequence omitted]

SEQ ID NO: 54 - protein, 11 aa, Artificial Sequence [sequence omitted]

SEQ ID NO: 55 - protein, 11 aa, Artificial Sequence [sequence omitted]

SEQ ID NO: 56 - protein, 10 aa, Artificial Sequence [sequence omitted]

SEQ ID NO: 57 - protein, 11 aa, Artificial Sequence
  Description of Artificial Sequence Synthetic peptide
CQQTYAIPLTF

SEQ ID NO: 58 - protein, 11 aa, Artificial Sequence [sequence omitted]

SEQ ID NO: 59 - protein, 11 aa, Artificial Sequence [sequence omitted]

SEQ ID NO: 60 - protein, 10 aa, Artificial Sequence [sequence omitted]

SEQ ID NO: 61 - protein, 11 aa, Artificial Sequence
  Description of Artificial Sequence Synthetic peptide
CQQSYSDQWTF

SEQ ID NO: 62 - protein, 11 aa, Artificial Sequence
  Description of Artificial Sequence Synthetic peptide
CQQSYLPPYSF

SEQ ID NO: 63 - protein, 11 aa, Artificial Sequence [sequence omitted]

SEQ ID NO: 64 - protein, 9 aa, Artificial Sequence
  Description of Artificial Sequence Synthetic peptide
CQEGITYTF

SEQ ID NO: 65 - protein, 11 aa, Artificial Sequence [sequence omitted]

SEQ ID NO: 66 - protein, 11 aa, Artificial Sequence
  Description of Artificial Sequence Synthetic peptide
CQHYGYSPVTF

SEQ ID NO: 67 - protein, 14 aa, Artificial Sequence
  Description of Artificial Sequence Synthetic peptide
CARDGVRYYGMDVW

SEQ ID NO: 68 - protein, 15 aa, Artificial Sequence
  Description of Artificial Sequence Synthetic peptide
CARGVRGYDRSAGYW

SEQ ID NO: 69 - protein, 16 aa, Artificial Sequence
  Description of Artificial Sequence Synthetic peptide
CASHDYGDYGEYFQHW

SEQ ID NO: 70 - protein, 21 aa, Artificial Sequence
  Description of Artificial Sequence Synthetic peptide
CARVSWYCSSTSCGVNWFDPW

SEQ ID NO: 71 - protein, 15 aa, Artificial Sequence
  Description of Artificial Sequence Synthetic peptide
CAKVNWNDGPYFDYW

SEQ ID NO: 72 - protein, 21 aa, Artificial Sequence
  Description of Artificial Sequence Synthetic peptide
CATPTNSGYYGPYYYYGMDVW

SEQ ID NO: 73 - protein, 9 aa, Artificial Sequence
  Description of Artificial Sequence Synthetic peptide
CARDVMDVW

SEQ ID NO: 74 - protein, 11 aa, Artificial Sequence [sequence omitted]

SEQ ID NO: 75 - protein, 12 aa, Artificial Sequence
  Description of Artificial Sequence Synthetic peptide
CARDNGVGVDYW

SEQ ID NO: 76 - protein, 25 aa, Artificial Sequence
  Description of Artificial Sequence Synthetic peptide
CARGIADSGSYYGNGRDYYYGMDVW

SEQ ID NO: 77 - protein, 11 aa, Artificial Sequence [sequence omitted]

SEQ ID NO: 78 - protein, 14 aa, Artificial Sequence
  Description of Artificial Sequence Synthetic peptide
CARDGTRYYGMDVW

SEQ ID NO: 79 - protein, 12 aa, Artificial Sequence
  Description of Artificial Sequence Synthetic peptide
CARDVVANFDYW

SEQ ID NO: 80 - protein, 18 aa, Artificial Sequence
  Description of Artificial Sequence Synthetic peptide
CARGHSSGWYYYYGMDVW

SEQ ID NO: 81 - protein, 13 aa, Artificial Sequence
  Description of Artificial Sequence Synthetic peptide
CAKDLGSYGGYYW

SEQ ID NO: 82 - protein, 19 aa, Artificial Sequence
  Description of Artificial Sequence Synthetic peptide
CARSWFGGFNYHYYGMDVW

SEQ ID NO: 83 - protein, 14 aa, Artificial Sequence
  Description of Artificial Sequence Synthetic peptide
CARELPIGYGMDVW

SEQ ID NO: 84 - protein, 15 aa, Artificial Sequence
  Description of Artificial Sequence Synthetic peptide
CARGGSYYYYGMDVW

SEQ ID NO: 85 - protein, 11 aa, Artificial Sequence [sequence omitted]

SEQ ID NO: 86 - protein, 11 aa, Artificial Sequence [sequence omitted]

SEQ ID NO: 87 - protein, 11 aa, Artificial Sequence [sequence omitted]

SEQ ID NO: 88 - protein, 11 aa, Artificial Sequence [sequence omitted]

SEQ ID NO: 89 - protein, 11 aa, Artificial Sequence
  Description of Artificial Sequence Synthetic peptide
CQQTYMMPYTF

SEQ ID NO: 90 - protein, 11 aa, Artificial Sequence [sequence omitted]

SEQ ID NO: 91 - protein, 11 aa, Artificial Sequence [sequence omitted]

SEQ ID NO: 92 - protein, 11 aa, Artificial Sequence [sequence omitted]

SEQ ID NO: 93 - protein, 11 aa, Artificial Sequence [sequence omitted]

SEQ ID NO: 94 - protein, 11 aa, Artificial Sequence [sequence omitted]

SEQ ID NO: 95 - protein, 11 aa, Artificial Sequence [sequence omitted]

SEQ ID NO: 96 - protein, 11 aa, Artificial Sequence [sequence omitted]

SEQ ID NO: 97 - protein, 11 aa, Artificial Sequence [sequence omitted]

SEQ ID NO: 98 - protein, 13 aa, Homo sapiens
KSSDKTHTCPPCP

SEQ ID NO: 99 - protein, 5 aa, Artificial Sequence
  Description of Artificial Sequence Synthetic peptide
GGGGS

SEQ ID NO: 100 - protein, 15 aa, Artificial Sequence
  Description of Artificial Sequence Synthetic peptide
GGGGSGGGGSGGGGS

SEQ ID NO: 101 - protein, 80 aa, Artificial Sequence
  Description of Artificial Sequence Synthetic polypeptide
  SITE (1)..(80): This sequence may encompass 1-20 “Gly Gly Gly Ser” repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 102 - protein, 80 aa, Artificial Sequence
  Description of Artificial Sequence Synthetic polypeptide
  SITE (1)..(80): This sequence may encompass 1-20 “Gly Gly Ser Gly” repeating units
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 103 - protein, 100 aa, Artificial Sequence
  Description of Artificial Sequence Synthetic polypeptide
  SITE (1)..(100): This sequence may encompass 1-20 “Gly Gly Ser Gly Gly” repeating units
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 104 - protein, 100 aa, Artificial Sequence
  Description of Artificial Sequence Synthetic polypeptide
  SITE (1)..(100): This sequence may encompass 1-20 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 105 - protein, 20 aa, Artificial Sequence
  Description of Artificial Sequence Synthetic peptide
  SITE (1)..(20): This sequence may encompass 1-4 “Gly Gly Gly Gly Ser” repeating units
GGGGSGGGGSGGGGSGGGGS

SEQ ID NO: 106 - protein, 10 aa, Artificial Sequence
  Description of Artificial Sequence Synthetic peptide
  SITE (1)..(10): This sequence may encompass 1-2 “Gly Gly Gly Gly Ser” repeating units
GGGGSGGGGS

SEQ ID NO: 107 - protein, 10 aa, Artificial Sequence
  Description of Artificial Sequence Synthetic peptide
GGGGSGGGGS

SEQ ID NO: 108 - protein, 50 aa, Artificial Sequence
  Description of Artificial Sequence Synthetic polypeptide
  SITE (1)..(50): This sequence may encompass 3-10 “Gly Gly Gly Gly Ser” repeating units
GGGGSGGGGSGGGGSGGGGSGGGGSGGGGSGGGGSGGGGSGGGGSGGGGS

SEQ ID NO: 109 - protein, 9 aa, Homo sapiens
EVDPIGHLY

SEQ ID NO: 110 - protein, 10 aa, Homo sapiens
GEMSSNSTAL

SEQ ID NO: 111 - protein, 10 aa, Homo sapiens
GVYDGEEHSV

SEQ ID NO: 112 - protein, 118 aa, Artificial Sequence
  Description of Artificial Sequence Synthetic polypeptide
QVQLVESGGGVVQPGRSLRLSCAASGFTFRSYGMHWVRQAPGKGLEWVAIIWYDGSKKNYADSVKGRFTISRDNSKNTLYLQMNSLRAEDTAVYYCARGTGYNWFDPWGQGTLVTVSS

SEQ ID NO: 113 - protein, 108 aa, Artificial Sequence
  Description of Artificial Sequence Synthetic polypeptide
EIVLTQSPRTLSLSPGERATLSCRASQSVSSSYLAWYQQKPGQAPRLLIYGASSRATGIPDRFSGSGSGTDFTLTISRLDPEDFAVYYCQQYGSSPITFGQGTRLEIK

SEQ ID NO: 114 - protein, 5 aa, Artificial Sequence
  Description of Artificial Sequence Synthetic peptide
SYGMH

SEQ ID NO: 115 - protein, 17 aa, Artificial Sequence
  Description of Artificial Sequence Synthetic peptide
IIWYDGSKKNYADSVKG

SEQ ID NO: 116 - protein, 9 aa, Artificial Sequence
  Description of Artificial Sequence Synthetic peptide
GTGYNWFDP

SEQ ID NO: 117 - protein, 12 aa, Artificial Sequence
  Description of Artificial Sequence Synthetic peptide
RASQSVSSSYLA

SEQ ID NO: 118 - protein, 7 aa, Artificial Sequence
  Description of Artificial Sequence Synthetic peptide
GASSRAT

SEQ ID NO: 119 - protein, 9 aa, Artificial Sequence
  Description of Artificial Sequence Synthetic peptide
QQYGSSPIT

SEQ ID NO: 120 - protein, 5 aa, Artificial Sequence
  Description of Artificial Sequence Synthetic peptide
RYTMH

SEQ ID NO: 121 - protein, 17 aa, Artificial Sequence
  Description of Artificial Sequence Synthetic peptide
YINPSRGYTNYNQKFKD

SEQ ID NO: 122 - protein, 10 aa, Artificial Sequence
  Description of Artificial Sequence Synthetic peptide
YYDDHYSLDY

SEQ ID NO: 123 - protein, 10 aa, Artificial Sequence
  Description of Artificial Sequence Synthetic peptide
SASSSVSYMN

SEQ ID NO: 124 - protein, 7 aa, Artificial Sequence
  Description of Artificial Sequence Synthetic peptide
DTSKLAS

SEQ ID NO: 125 - protein, 9 aa, Artificial Sequence
  Description of Artificial Sequence Synthetic peptide
QQWSSNPFT

SEQ ID NO: 126 - protein, 119 aa, Artificial Sequence
  Description of Artificial Sequence Synthetic polypeptide
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSS

SEQ ID NO: 127 - protein, 106 aa, Artificial Sequence
  Description of Artificial Sequence Synthetic polypeptide
DIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 128 - protein, 9 aa, Artificial Sequence
  Description of Artificial Sequence Synthetic peptide
YTFTRYTMH

SEQ ID NO: 129 - protein, 13 aa, Artificial Sequence
  Description of Artificial Sequence Synthetic peptide
GYINPSRGYTNYN

SEQ ID NO: 130 - protein, 14 aa, Artificial Sequence
  Description of Artificial Sequence Synthetic peptide
CARYYDDHYSLDYW

SEQ ID NO: 131 - protein, 11 aa, Artificial Sequence
  Description of Artificial Sequence Synthetic peptide
CQQWSSNPFTF

SEQ ID NO: 132 - protein, 125 aa, Artificial Sequence
  Description of Artificial Sequence Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 133 - protein, 109 aa, Artificial Sequence
  Description of Artificial Sequence Synthetic polypeptide
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 134 - protein, 20 aa, Artificial Sequence
  Description of Artificial Sequence Synthetic peptide
FTFSTYAMNWVRQAPGKGLE

SEQ ID NO: 135 - protein, 16 aa, Artificial Sequence
  Description of Artificial Sequence Synthetic peptide
TYYADSVKGRFTISRD

SEQ ID NO: 136 - protein, 18 aa, Artificial Sequence
  Description of Artificial Sequence Synthetic peptide
CVRHGNFGDSYVSWFAYW

SEQ ID NO: 137 - protein, 14 aa, Artificial Sequence
  Description of Artificial Sequence Synthetic peptide
GSSTGAVTTSNYAN

SEQ ID NO: 138 - protein, 7 aa, Artificial Sequence
  Description of Artificial Sequence Synthetic peptide
GTNKRAP

SEQ ID NO: 139 - protein, 11 aa, Artificial Sequence
  Description of Artificial Sequence Synthetic peptide
CALWYSNHWVF

SEQ ID NO: 140 - protein, 50 aa, Artificial Sequence; identical to SEQ ID NO: 108
  Description of Artificial Sequence Synthetic polypeptide
  SITE (1)..(50): This sequence may encompass 1-10 “Gly Gly Gly Gly Ser” repeating units

SEQ ID NO: 141 - protein, 119 aa, Artificial Sequence
  Description of Artificial Sequence Synthetic polypeptide
QVQLVQSGAEVKKPGASVKVSCKASGYTFTSYDINWVRQAPGQGLEWMGIINPRSGSTKYAQKFQGRVTMTRDTSTSTVYMELSSLRSEDTAVYYCARDGVRYYGMDVWGQGTTVTVSS

SEQ ID NO: 142 - protein, 120 aa, Artificial Sequence
  Description of Artificial Sequence Synthetic polypeptide
QVQLVQSGAEVKKPGSSVKVSCKASGYTFTSHDINWVRQAPGQGLEWMGWMNPNSGDTGYAQKFQGRVTITADESTSTAYMELSSLRSEDTAVYYCARGVRGYDRSAGYWGQGTLVIVSS

SEQ ID NO: 143 - protein, 121 aa, Artificial Sequence
  Description of Artificial Sequence Synthetic polypeptide
EVQLLESGGGLVKPGGSLRLSCAASGFSFSSYWMSWVRQAPGKGLEWISYISGDSGYTNYADSVKGRFTISRDDSKNTLYLQMNSLKTEDTAVYYCASHDYGDYGEYFQHWGQGTLVTVSS

SEQ ID NO: 144 - protein, 126 aa, Artificial Sequence
  Description of Artificial Sequence Synthetic polypeptide
EVQLLQSGGGLVQPGGSLRLSCAASGFTFSNSDMNWVRQAPGKGLEWVAYISSGSSTIYYADSVKGRFTISRDNSKNTLYLQMNSLRAEDTAVYYCARVSWYCSSTSCGVNWFDPWGQGTLVTVSS

SEQ ID NO: 145 - protein, 120 aa, Artificial Sequence
  Description of Artificial Sequence Synthetic polypeptide
EVQLLESGGGLVQPGGSLRLSCAASGFTFSNSDMNWVRQAPGKGLEWVASISSSGGYINYADSVKGRFTISRDNSKNTLYLQMNSLRAEDTAVYYCAKVNWNDGPYFDYWGQGTLVTVSS

SEQ ID NO: 146 - protein, 126 aa, Artificial Sequence
  Description of Artificial Sequence Synthetic polypeptide
QVQLVQSGAEVKKPGSSVKVSCKASGGTFSNFGVSWLRQAPGQGLEWMGGIIPILGTANYAQKFQGRVTITADESTSTAYMELSSLRSEDTAVYYCATPTNSGYYGPYYYYGMDVWGQGTTVTVSS

SEQ ID NO: 147 - protein, 114 aa, Artificial Sequence
  Description of Artificial Sequence Synthetic polypeptide
QVQLVQSGAEVKKPGASVKVSCKASGYTFTSYNMHWVRQAPGQGLEWMGWINPNSGGTNYAQKFQGRVTMTRDTSTSTVYMELSSLRSEDTAVYYCARDVMDVWGQGTTVTVSS

SEQ ID NO: 148 - protein, 116 aa, Artificial Sequence
  Description of Artificial Sequence Synthetic polypeptide
QVQLVQSGAEVKKPGASVKVSCKASGGTFSGYLVSWVRQAPGQGLEWMGWINPNSGGTNTAQKFQGRVTMTRDTSTSTVYMELSSLRSEDTAVYYCAREGYGMDVWGQGTTVTVSS

SEQ ID NO: 149 - protein, 117 aa, Artificial Sequence
  Description of Artificial Sequence Synthetic polypeptide
QVQLVQSGAEVKKPGASVKVSCKASGYIFRNYPMHWVRQAPGQGLEWMGWINPDSGGTKYAQKFQGRVTMTRDTSTSTVYMELSSLRSEDTAVYYCARDNGVGVDYWGQGTLVTVSS

SEQ ID NO: 150 - protein, 130 aa, Artificial Sequence
  Description of Artificial Sequence Synthetic polypeptide
QVQLVQSGAEVKKPGASVKVSCKASGYTFTGYYMHWVRQAPGQGLEWMGWMNPNIGNTGYAQKFQGRVTMTRDTSTSTVYMELSSLRSEDTAVYYCARGIADSGSYYGNGRDYYYGMDVWGQGTTVTVSS

SEQ ID NO: 151 - protein, 116 aa, Artificial Sequence
  Description of Artificial Sequence Synthetic polypeptide
QVQLVQSGAEVKKPGASVKVSCKASGGTFSSYGISWVRQAPGQGLEWMGWINPNSGVTKYAQKFQGRVTMTRDTSTSTVYMELSSLRSEDTAVYYCARGDYYFDYWGQGTLVTVSS

SEQ ID NO: 152 - protein, 119 aa, Artificial Sequence
  Description of Artificial Sequence Synthetic polypeptide
QVQLVQSGAEVKKPGASVKVSCKASGYTFTSYDINWVRQAPGQGLEWMGWINPNSGDTKYSQKFQGRVTMTRDTSTSTVYMELSSLRSEDTAVYYCARDGTRYYGMDVWGQGTTVTVSS

SEQ ID NO: 153 - protein, 117 aa, Artificial Sequence
  Description of Artificial Sequence Synthetic polypeptide
EVQLLESGGGLVKPGGSLRLSCAASGFTFSDYYMSWVRQAPGKGLEWVSYISSSSSYTNYADSVKGRFTISRDDSKNTLYLQMNSLKTEDTAVYYCARDVVANFDYWGQGTLVTVSS

SEQ ID NO: 154 - protein, 123 aa, Artificial Sequence
  Description of Artificial Sequence Synthetic polypeptide
QVQLVQSGAEVKKPGASVKVSCKASGGTFSSYAISWVRQAPGQGLEWMGWMNPDSGSTGYAQRFQGRVTMTRDTSTSTVYMELSSLRSEDTAVYYCARGHSSGWYYYYGMDVWGQGTTVTVSS

SEQ ID NO: 155 - protein, 118 aa, Artificial Sequence
  Description of Artificial Sequence Synthetic polypeptide
EVQLLESGGGLVQPGGSLRLSCAASGFTFTSYSMHWVRQAPGKGLEWVSSITSFTNTMYYADSVKGRFTISRDNSKNTLYLQMNSLRAEDTAVYYCAKDLGSYGGYYWGQGTLVTVSS

SEQ ID NO: 156 - protein, 124 aa, Artificial Sequence
  Description of Artificial Sequence Synthetic polypeptide
QVQLVQSGAEVKKPGASVKVSCKASGYTFTNYYMHWVRQAPGQGLEWMGIINPSGGSTSYAQKFQGRVTMTRDTSTSTVYMELSSLRSEDTAVYYCARSWFGGFNYHYYGMDVWGQGTTVTVSS

SEQ ID NO: 157 - protein, 119 aa, Artificial Sequence
  Description of Artificial Sequence Synthetic polypeptide
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARELPIGYGMDVWGQGTTVTVSS

SEQ ID NO: 158 - protein, 120 aa, Artificial Sequence
  Description of Artificial Sequence Synthetic polypeptide
QVQLVQSGAEVKKPGSSVKVSCKASGGTFSSYAISWVRQAPGQGLEWMGGIIPIVGTANYAQKFQGRVTITADESTSTAYMELSSLRSEDTAVYYCARGGSYYYYGMDVWGQGTTVTVSS

SEQ ID NO: 159 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSYRASGVPDRFSGSGSGTDFTLKISRVEAEDVGVYYCMQGLQTPITFGQGTRLEIK

SEQ ID NO: 160 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSSRASGVPDRFSGSGSGTDFTLKISRVEAEDVGVYYCMQALQTPPTFGPGTKVDIK

SEQ ID NO: 161 - protein, 107 aa, Artificial Sequence
  Description of Artificial Sequence Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQAISFPLTFGQSTKVEIK

SEQ ID NO: 162 - protein, 107 aa, Artificial Sequence
  Description of Artificial Sequence Synthetic polypeptide
DIQMTQSPSSLSASVGDRVTITCRASQSISSWLAWYQQKPGKAPKLLIYSASTLQSGVPSRFSGSGSGTDFTLTISSLQPEDFATYYCQQANSFPLTFGGGTKVEIK

SEQ ID NO: 163 - protein, 107 aa, Artificial Sequence
  Description of Artificial Sequence Synthetic polypeptide
DIQMTQSPSSLSASVGDRVTITCRASQSISSWLAWYQQKPGKAPKLLIYAASSLQSGVPSRFSGSGSGTDFTLTISSLQPEDFATYYCQQANSFPLTFGGGTKVEIK

SEQ ID NO: 164 - protein, 107 aa, Artificial Sequence
  Description of Artificial Sequence Synthetic polypeptide
DIQMTQSPSSLSASVGDRVTITCRASQSISSWLAWYQQKPGKAPKLLIYAASTLQSGVPSRFSGSGSGTDFTLTISSLQPEDFATYYCQQSYSIPLTFGGGTKVEIK

SEQ ID NO: 165 - protein, 107 aa, Artificial Sequence
  Description of Artificial Sequence Synthetic polypeptide
DIQMTQSPSSLSASVGDRVTITCRASQGISNYLNWYQQKPGKAPKLLIYYASSLQSGVPSRFSGSGSGTDFTLTISSLQPEDFATYYCQQTYMMPYTFGQGTKVEIK

SEQ ID NO: 166 - protein, 107 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQSYITPWTFGQGTKVEIK

SEQ ID NO: 167 - protein, 107 aa, Artificial Sequence
  Description of Artificial Sequence Synthetic polypeptide
DIQMTQSPSSLSASVGDRVTITCRASQGISNYLAWYQQKPGKAPKLLIYAASSLQSGVPSRFSGSGSGTDFTLTISSLQPEDFATYYCQQSYITPYTFGQGTKLEIK

SEQ ID NO: 168 - protein, 113 aa, Artificial Sequence
  Description of Artificial Sequence Synthetic polypeptide
DIVMTQSPDSLAVSLGERATINCKTSQSVLYRPNNENYLAWYQQKPGQPPKLLIYQASIREPGVPDRFSGSGSGTDFTLTISSLQAEDVAVYYCQQYYTTPYTFGQGTKLEIK

SEQ ID NO: 169 - protein, 107 aa, Artificial Sequence
  Description of Artificial Sequence Synthetic polypeptide
DIQMTQSPSSLSASVGDRVTITCRASQSISRFLNWYQQKPGKAPKLLIYGASRPQSGVPSRFSGSGSGTDFTLTISSLQPEDFATYYCQQSYSTPLTFGQGTKVEIK

SEQ ID NO: 170 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSHRASGVPDRFSGSGSGTDFTLKISRVEAEDVGVYYCMQALQTPLTFGGGTKVEIK

SEQ ID NO: 171 - protein, 107 aa, Artificial Sequence
  Description of Artificial Sequence Synthetic polypeptide
EIVMTQSPATLSVSPGERATLSCRASQSVSSNLAWYQQKPGQAPRLLIYAASARASGIPARFSGSGSGTEFTLTISSLQSEDFAVYYCQQYGSWPRTFGQGTKVEIK

SEQ ID NO: 172 - protein, 107 aa, Artificial Sequence
  Description of Artificial Sequence Synthetic polypeptide
DIQMTQSPSSLSASVGDRVTITCRASQSISSYLNWYQQKPGKAPKLLIYGASRLQSGVPSRFSGSGSGTDFTLTISSLQPEDFATYYCQQSYSTPVTFGQGTKVEIK

SEQ ID NO: 173 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKVEIK

SEQ ID NO: 174 - protein, 107 aa, Artificial Sequence
  Description of Artificial Sequence Synthetic polypeptide
DIQMTQSPSSLSASVGDRVTITCQASEDISNHLNWYQQKPGKAPKLLIYDALSLQSGVPSRFSGSGSGTDFTLTISSLQPEDFATYYCQQANSFPFTFGPGTKVDIK

SEQ ID NO: 175 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGQGTKVEIK

SEQ ID NO: 176 - protein, 107 aa, Artificial Sequence [sequence omitted]

SEQ ID NO: 177 - protein, 16 aa, Artificial Sequence
  Description of Artificial Sequence Synthetic peptide
CARDDYGDYVAYFQHW

SEQ ID NO: 178 - protein, 14 aa, Artificial Sequence [sequence omitted]

SEQ ID NO: 179 - protein, 17 aa, Artificial Sequence
  Description of Artificial Sequence Synthetic peptide
CARVYDFWSVLSGFDIW

SEQ ID NO: 180 - protein, 19 aa, Artificial Sequence
  Description of Artificial Sequence Synthetic peptide
CARVEQGYDIYYYYYMDVW

SEQ ID NO: 181 - protein, 16 aa, Artificial Sequence
  Description of Artificial Sequence Synthetic peptide
CARSYDYGDYLNFDYW

SEQ ID NO: 182 - protein, 18 aa, Artificial Sequence
  Description of Artificial Sequence Synthetic peptide
CARASGSGYYYYYGMDVW

SEQ ID NO: 183 - protein, 13 aa, Artificial Sequence
  Description of Artificial Sequence Synthetic peptide
CAASTWIQPFDYW

SEQ ID NO: 184 - protein, 17 aa, Artificial Sequence
  Description of Artificial Sequence Synthetic peptide
CASNGNYYGSGSYYNYW

SEQ ID NO: 185 - protein, 17 aa, Artificial Sequence
  Description of Artificial Sequence Synthetic peptide
CARAVYYDFWSGPFDYW

SEQ ID NO: 186 - protein, 16 aa, Artificial Sequence
  Description of Artificial Sequence Synthetic peptide
CAKGGIYYGSGSYPSW

SEQ ID NO: 187 - protein, 11 aa, Artificial Sequence [sequence omitted]

SEQ ID NO: 188 - protein, 18 aa, Artificial Sequence
  Description of Artificial Sequence Synthetic peptide
CARGLYGDYFLYYGMDVW

SEQ ID NO: 189 - protein, 19 aa, Artificial Sequence
  Description of Artificial Sequence Synthetic peptide
CARGLLGFGEFLTYGMDVW

SEQ ID NO: 190 - protein, 19 aa, Artificial Sequence
  Description of Artificial Sequence Synthetic peptide
CARDRDSSWTYYYYGMDVW

SEQ ID NO: 191 - protein, 21 aa, Artificial Sequence
  Description of Artificial Sequence Synthetic peptide
CARGDYYDSSGYYFPVYFDYW

SEQ ID NO: 192 - protein, 19 aa, Artificial Sequence
  Description of Artificial Sequence Synthetic peptide
CAKDPFWSGHYYYYGMDVW

SEQ ID NO: 193 - protein, 239 aa, Artificial Sequence
  Description of Artificial Sequence Synthetic polypeptide
QVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIKGGC

SEQ ID NO: 194 - protein, 10 aa, Artificial Sequence
  Description of Artificial Sequence Synthetic peptide
CQQNYNSVTF

SEQ ID NO: 195 - protein, 11 aa, Artificial Sequence [sequence omitted]

SEQ ID NO: 196 - protein, 11 aa, Artificial Sequence
  Description of Artificial Sequence Synthetic peptide
CGQSYSTPPTF

SEQ ID NO: 197 - protein, 11 aa, Artificial Sequence [sequence omitted]

SEQ ID NO: 198 - protein, 11 aa, Artificial Sequence [sequence omitted]

SEQ ID NO: 199 - protein, 11 aa, Artificial Sequence
  Description of Artificial Sequence Synthetic peptide
CQQYSTYPITI

SEQ ID NO: 200 - protein, 11 aa, Artificial Sequence [sequence omitted]

SEQ ID NO: 201 - protein, 11 aa, Artificial Sequence [sequence omitted]

SEQ ID NO: 202 - protein, 11 aa, Artificial Sequence [sequence omitted]

SEQ ID NO: 203 - protein, 11 aa, Artificial Sequence [sequence omitted]

SEQ ID NO: 204 - protein, 11 aa, Artificial Sequence [sequence omitted]

SEQ ID NO: 205 - protein, 11 aa, Artificial Sequence [sequence omitted]

SEQ ID NO: 206 - protein, 121 aa, Artificial Sequence
  Description of Artificial Sequence Synthetic polypeptide
QVQLVQSGAEVKKPGASVKVSCKASGGTFSRSAITWVRQAPGQGLEWMGWINPNSGATNYAQKFQGRVTMTRDTSTSTVYMELSSLRSEDTAVYYCARDDYGDYVAYFQHWGQGTLVTVSS

SEQ ID NO: 207 - protein, 119 aa, Artificial Sequence
  Description of Artificial Sequence Synthetic polypeptide
QVQLVQSGAEVKKPGASVKVSCKASGYPFIGQYLHWVRQAPGQGLEWMGIINPSGDSATYAQKFQGRVTMTRDTSTSTVYMELSSLRSEDTAVYYCARDLSYYYGMDVWGQGTTVTVSS

SEQ ID NO: 208 - protein, 122 aa, Artificial Sequence
  Description of Artificial Sequence Synthetic polypeptide
QVQLVQSGAEVKKPGASVKVSCKASGYTFTNYYMHWVRQAPGQGLEWMGWMNPIGGGTGYAQKFQGRVTMTRDTSTSTVYMELSSLRSEDTAVYYCARVYDFWSVLSGFDIWGQGTLVTVSS

SEQ ID NO: 209 - protein, 124 aa, Artificial Sequence
  Description of Artificial Sequence Synthetic polypeptide
EVQLLESGGGLVQPGGSLRLSCAASGFTFSDYYMSWVRQAPGKGLEWVSGINWNGGSTGYADSVKGRFTISRDNSKNTLYLQMNSLRAEDTAVYYCARVEQGYDIYYYYYMDVWGKGTTVTVSS

SEQ ID NO: 210 - protein, 121 aa, Artificial Sequence
  Description of Artificial Sequence Synthetic polypeptide
QVQLVQSGAEVKKPGASVKVSCKASGGTLSSYPINWVRQAPGQGLEWMGWISTYSGHADYAQKLQGRVTMTRDTSTSTVYMELSSLRSEDTAVYYCARSYDYGDYLNFDYWGQGTLVTVSS

SEQ ID NO: 211 - protein, 123 aa, Artificial Sequence
  Description of Artificial Sequence Synthetic polypeptide
EVQLLESGGGLVQPGGSLRLSCAASGFTFSSYWMSWVRQAPGKGLEWVSSISGRGDNTYYADSVKGRFTISRDNSKNTLYLQMNSLRAEDTAVYYCARASGSGYYYYYGMDVWGQGTTVTVSS

SEQ ID NO: 212 - protein, 118 aa, Artificial Sequence
  Description of Artificial Sequence Synthetic polypeptide
QVQLVQSGAEVKKPGASVKVSCKASGYTFGNYFMHWVRQAPGQGLEWMGMVNPSGGSETFAQKFQGRVTMTRDTSTSTVYMELSSLRSEDTAVYYCAASTWIQPFDYWGQGTLVTVSS

SEQ ID NO: 213 - protein, 122 aa, Artificial Sequence
  Description of Artificial Sequence Synthetic polypeptide
EVQLLESGGGLVQPGGSLRLSCAASGFDFSIYSMNWVRQAPGKGLEWVSAISGSGGSTYYADSVKGRFTISRDNSKNTLYLQMNSLRAEDTAVYYCASNGNYYGSGSYYNYWGQGTLVTVSS

SEQ ID NO: 214 - protein, 122 aa, Artificial Sequence
  Description of Artificial Sequence Synthetic polypeptide
QVQLVQSGAEVKKPGASVKVSCKASGYTLTTYYMHWVRQAPGQGLEWMGWINPNSGGTNYAQKFQGRVTMTRDTSTSTVYMELSSLRSEDTAVYYCARAVYYDFWSGPFDYWGQGTLVTVSS

SEQ ID NO: 215 - protein, 121 aa, Artificial Sequence
  Description of Artificial Sequence Synthetic polypeptide
QVQLVQSGAEVKKPGASVKVSCKASGYTFTSYYMHWVRQAPGQGLEWMGWINPYSGGTNYAQKFQGRVTMTRDTSTSTVYMELSSLRSEDTAVYYCAKGGIYYGSGSYPSWGQGTLVTVSS

SEQ ID NO: 216 - protein, 116 aa, Artificial Sequence
  Description of Artificial Sequence Synthetic polypeptide
QVQLVQSGAEVKKPGSSVKVSCKASGGTFSSYGVSWVRQAPGQGLEWMGWISPYSGNTDYAQKFQGRVTITADESTSTAYMELSSLRSEDTAVYYCARGLYYMDVWGKGTTVTVSS

SEQ ID NO: 217 - protein, 123 aa, Artificial Sequence
  Description of Artificial Sequence Synthetic polypeptide
QVQLVQSGAEVKKPGASVKVSCKASGYTFSNMYLHWVRQAPGQGLEWMGWINPNTGDTNYAQTFQGRVTMTRDTSTSTVYMELSSLRSEDTAVYYCARGLYGDYFLYYGMDVWGQGTKVTVSS

SEQ ID NO: 218 - protein, 124 aa, Artificial Sequence
  Description of Artificial Sequence Synthetic polypeptide
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARGLLGFGEFLTYGMDVWGQGTLVTVSS

SEQ ID NO: 219 - protein, 124 aa, Artificial Sequence
  Description of Artificial Sequence Synthetic polypeptide
QVQLVQSGAEVKKPGASVKVSCKASGYTFTGYYIHWVRQAPGQGLEWMGVINPSGGSTTYAQKLQGRVTMTRDTSTSTVYMELSSLRSEDTAVYYCARDRDSSWTYYYYGMDVWGQGTTVTVSS

SEQ ID NO: 220 - protein, 123 aa, Artificial Sequence
  Description of Artificial Sequence Synthetic polypeptide
QVQLVQSGAEVKKPGASVKVSCKASGYTFTSNYMHWVRQAPGQGLEWMGWMNPNSGNTGYAQKFQGRVTMTRDTSTSTVYMELSSLRSEDTAVYYCARGLYGDYFLYYGMDVWGQGTTVTVSS

SEQ ID NO: 221 - protein, 125 aa, Artificial Sequence
  Description of Artificial Sequence Synthetic polypeptide
QVQLVQSGAEVKKPGASVKVSCKASGGTFSSHAISWVRQAPGQGLEWMGVIIPSGGTSYTQKFQGRVTMTRDTSTSTVYMELSSLRSEDTAVYYCARGDYYDSSGYYFPVYFDYWGQGTLVTVSS

SEQ ID NO: 222 - protein, 124 aa, Artificial Sequence
  Description of Artificial Sequence Synthetic polypeptide
QVQLVQSGAEVKKPGASVKVSCKASGYTFTSYAMNWVRQAPGQGLEWMGWINPNSGGTNYAQKFQGRVTMTRDTSTSTVYMELSSLRSEDTAVYYCARDPFWSGHYYYYGMDVWGQGTTVTVSS

SEQ ID NO: 223 - protein, 106 aa, Artificial Sequence
  Description of Artificial Sequence Synthetic polypeptide
DIQMTQSPSSLSASVGDRVTITCRASQSITSYLNWYQQKPGKAPKLLIYDASNLETGVPSRFSGSGSGTDFTLTISSLQPEDFATYYCQQNYNSVTFGQGTKLEIK

SEQ ID NO: 224 - protein, 107 aa, Artificial Sequence
  Description of Artificial Sequence Synthetic polypeptide
DIQMTQSPSSLSASVGDRVTITCWASQGISSYLAWYQQKPGKAPKLLIYAASSLQSGVPSRFSGSGSGTDFTLTISSLQPEDFATYYCQQSYNTPWTFGPGTKVDIK

SEQ ID NO: 225 - protein, 107 aa, Artificial Sequence
  Description of Artificial Sequence Synthetic polypeptide
DIQMTQSPSSLSASVGDRVTITCRASQAISNSLAWYQQKPGKAPKLLIYAASTLQSGVPSRFSGSGSGTDFTLTISSLQPEDFATYYCGQSYSTPPTFGQGTKLEIK

SEQ ID NO: 226 - protein, 107 aa, Artificial Sequence
  Description of Artificial Sequence Synthetic polypeptide
DIQMTQSPSSLSASVGDRVTITCRASQSISSYLNWYQQKPGKAPKLLIYKASSLESGVPSRFSGSGSGTDFTLTISSLQPEDFATYYCQQSYSAPYTFGPGTKVDIK

SEQ ID NO: 227 - protein, 107 aa, Artificial Sequence
  Description of Artificial Sequence Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQSYSIPPTFGGGTKVDIK

SEQ ID NO: 228 - protein, 107 aa, Artificial Sequence
  Description of Artificial Sequence Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQSYSAPYTFGGGTKVEIK

SEQ ID NO: 229 - protein, 107 aa, Artificial Sequence
  Description of Artificial Sequence Synthetic polypeptide
DIQMTQSPSSLSASVGDRVTITCRASQGINSYLAWYQQKPGKAPKLLIYDASNLETGVPSRFSGSGSGTDFTLTISSLQPEDFATYYCQQHNSYPPTFGQGTKLEIK

SEQ ID NO: 230 - protein, 107 aa, Artificial Sequence
  Description of Artificial Sequence Synthetic polypeptide
DIQMTQSPSSLSASVGDRVTITCRASQSISRWLAWYQQKPGKAPKLLIYAASSLQSGVPSRFSGSGSGTDFTLTISSLQPEDFATYYCQQYSTYPITIGQGTKVEIK

SEQ ID NO: 231 - protein, 107 aa, Artificial Sequence
  Description of Artificial Sequence Synthetic polypeptide
DIQMTQSPSSLSASVGDRVTITCRASQGISNSLAWYQQKPGKAPKLLIYAASSLQSGVPSRFSGSGSGTDFTLTISSLQPEDFATYYCQQANSFPWTFGQGTKLEIK

SEQ ID NO: 232 - protein, 107 aa, Artificial Sequence
  Description of Artificial Sequence Synthetic polypeptide
DIQMTQSPSSLSASVGDRVTITCRASQDVSTWLAWYQQKPGKAPKLLIYAASSLQSGVPSRFSGSGSGTDFTLTISSLQPEDFATYYCQQSHSTPQTFGQGTKVEIK

SEQ ID NO: 233 - protein, 107 aa, Artificial Sequence
  Description of Artificial Sequence Synthetic polypeptide
DIQMTQSPSSLSASVGDRVTITCRASQSISSWLAWYQQKPGKAPKLLIYDASNLETGVPSRFSGSGSGTDFTLTISSLQPEDFATYYCQQSYSTPLTFGGGTKLEIK

SEQ ID NO: 234 - protein, 107 aa, Artificial Sequence
  Description of Artificial Sequence Synthetic polypeptide
DIQMTQSPSSLSASVGDRVTITCRASQGISNYLAWYQQKPGKAPKLLIYAASTLQSGVPSRFSGSGSGTDFTLTISSLQPEDFATYYCQQSYSTPLTFGGGTKVEIK

SEQ ID NO: 235 - protein, 107 aa, Artificial Sequence
  Description of Artificial Sequence Synthetic polypeptide
DIQMTQSPSSLSASVGDRVTITCRASQGISNWLAWYQQKPGKAPKLLIYAASTLQSGVPSRFSGSGSGTDFTLTISSLQPEDFATYYCQQTYSTPWTFGQGTKLEIK

SEQ ID NO: 236 - protein, 107 aa, Artificial Sequence
  Description of Artificial Sequence Synthetic polypeptide
EIVMTQSPATLSVSPGERATLSCRASQSVGNSLAWYQQKPGQAPRLLIYGASTRATGIPARFSGSGSGTEFTLTISSLQSEDFAVYYCQQYGSSPYTFGQGTKVEIK

SEQ ID NO: 237 - protein, 107 aa, Artificial Sequence
  Description of Artificial Sequence Synthetic polypeptide
DIQMTQSPSSLSASVGDRVTITCRASQSISGYLNWYQQKPGKAPKLLIYAASSLQSGVPSRFSGSGSGTDFTLTISSLQPEDFATYYCQQSHSTPLTFGQGTKVEIK

SEQ ID NO: 238 - protein, 107 aa, Artificial Sequence
  Description of Artificial Sequence Synthetic polypeptide
DIQMTQSPSSLSASVGDRVTITCRASQNIYTYLNWYQQKPGKAPKLLIYDASNLETGVPSRFSGSGSGTDFTLTISSLQPEDFATYYCQQANGFPLTFGGGTKVEIK

SEQ ID NO: 239 - protein, 19 aa, Artificial Sequence
  Description of Artificial Sequence Synthetic peptide
CARDQDTIFGVVITWFDPW

SEQ ID NO: 240 - protein, 14 aa, Artificial Sequence
  Description of Artificial Sequence Synthetic peptide
CARDKVYGDGFDPW

SEQ ID NO: 241 - protein, 11 aa, Artificial Sequence
  Description of Artificial Sequence Synthetic peptide
CAREDDSMDVW

SEQ ID NO: 242 - protein, 11 aa, Artificial Sequence
  Description of Artificial Sequence Synthetic peptide
CARDSSGLDPW

SEQ ID NO: 243 - protein, 11 aa, Artificial Sequence [sequence omitted]

SEQ ID NO: 244 - protein, 27 aa, Artificial Sequence
  Description of Artificial Sequence Synthetic peptide
CARDAHQYYDFWSGYYSGTYYYGMDVW

SEQ ID NO: 245 - protein, 15 aa, Artificial Sequence
  Description of Artificial Sequence Synthetic peptide
CAREQWPSYWYFDLW

SEQ ID NO: 246 - protein, 15 aa, Artificial Sequence [sequence omitted]

SEQ ID NO: 247 - protein, 19 aa, Artificial Sequence
  Description of Artificial Sequence Synthetic peptide
CARGSGDPNYYYYYGLDVW

SEQ ID NO: 248 - protein, 12 aa, Artificial Sequence
  Description of Artificial Sequence Synthetic peptide
CARDTGDHFDYW

SEQ ID NO: 249 - protein, 11 aa, Artificial Sequence
  Description of Artificial Sequence Synthetic peptide
CARAENGMDVW

SEQ ID NO: 250 - protein, 12 aa, Artificial Sequence
  Description of Artificial Sequence Synthetic peptide
CARDPGGYMDVW

SEQ ID NO: 251 - protein, 11 aa, Artificial Sequence [sequence omitted]

SEQ ID NO: 252 - protein, 12 aa, Artificial Sequence
  Description of Artificial Sequence Synthetic peptide
CARDMGDAFDIW

SEQ ID NO: 253 - protein, 11 aa, Artificial Sequence
  Description of Artificial Sequence Synthetic peptide
CAREEDGMDVW

SEQ ID NO: 254 - protein, 19 aa, Artificial Sequence
  Description of Artificial Sequence Synthetic peptide
CARGEYSSGFFFVGWFDLW

SEQ ID NO: 255 - protein, 13 aa, Artificial Sequence
  Description of Artificial Sequence Synthetic peptide
CARETGDDAFDIW

SEQ ID NO: 256 - protein, 11 aa, Artificial Sequence [sequence omitted]

SEQ ID NO: 257 - protein, 11 aa, Artificial Sequence
  Description of Artificial Sequence Synthetic peptide
CQQAEAFPYTF

SEQ ID NO: 258 - protein, 11 aa, Artificial Sequence [sequence omitted]

SEQ ID NO: 259 - protein, 11 aa, Artificial Sequence [sequence omitted]

SEQ ID NO: 260 - protein, 11 aa, Artificial Sequence
  Description of Artificial Sequence Synthetic peptide
CHQTYSTPLTF

SEQ ID NO: 261 - protein, 11 aa, Artificial Sequence
  Description of Artificial Sequence Synthetic peptide
CQQAYSFPWTF

SEQ ID NO: 262 - protein, 11 aa, Artificial Sequence [sequence omitted]

SEQ ID NO: 263 - protein, 11 aa, Artificial Sequence [sequence omitted]

SEQ ID NO: 264 - protein, 11 aa, Artificial Sequence [sequence omitted]

SEQ ID NO: 265 - protein, 10 aa, Artificial Sequence
  Description of Artificial Sequence Synthetic peptide
CQQSYGVPTF

SEQ ID NO: 266 - protein, 11 aa, Artificial Sequence [sequence omitted]

SEQ ID NO: 267 - protein, 11 aa, Artificial Sequence
  Description of Artificial Sequence Synthetic peptide
CMQTLKTPLSF

SEQ ID NO: 268 - protein, 124 aa, Artificial Sequence
  Description of Artificial Sequence Synthetic polypeptide
EVQLLESGGGLVKPGGSLRLSCAASGFTFSSYWMSWVRQAPGKGLEWVSGISARSGRTYYADSVKGRFTISRDDSKNTLYLQMNSLKTEDTAVYYCARDQDTIFGVVITWFDPWGQGTLVTVSS

SEQ ID NO: 269 - protein, 119 aa, Artificial Sequence
  Description of Artificial Sequence Synthetic polypeptide
QVQLVQSGAEVKKPGASVKVSCKASGYTFTSYYMHWVRQAPGQGLEWMGIIHPGGGTTSYAQKFQGRVTMTRDTSTSTVYMELSSLRSEDTAVYYCARDKVYGDGFDPWGQGTLVTVSS

SEQ ID NO: 270 - protein, 116 aa, Artificial Sequence
  Description of Artificial Sequence Synthetic polypeptide
QVQLVQSGAEVKKPGASVKVSCKASGYIFTGYYMHWVRQAPGQGLEWMGMIGPSDGSTSYAQKFQGRVTMTRDTSTSTVYMELSSLRSEDTAVYYCAREDDSMDVWGKGTTVTVSS

SEQ ID NO: 271 - protein, 116 aa, Artificial Sequence
  Description of Artificial Sequence Synthetic polypeptide
QVQLVQSGAEVKKPGASVKVSCKASGYTFIGYYMHWVRQAPGQGLEWMGMIGPSDGSTSYAQKFQGRVTMTRDTSTSTVYMELSSLRSEDTAVYYCARDSSGLDPWGQGTLVTVSS

SEQ ID NO: 272 - protein, 116 aa, Artificial Sequence
  Description of Artificial Sequence Synthetic polypeptide
QVQLVQSGAEVKKPGASVKVSCKASGYTFTGYYMHWVRQAPGQGLEWMGMIGPSDGSTSYAQKFQGRVTMTRDTSTSTVYMELSSLRSEDTAVYYCARGVGNLDYWGQGTLVTVSS

SEQ ID NO: 273 - protein, 132 aa, Artificial Sequence
  Description of Artificial Sequence Synthetic polypeptide
QVQLVQSGAEVKKPGASVKVSCKASGVTFSTSAISWVRQAPGQGLEWMGWISPYNGNTDYAQMLQGRVTMTRDTSTSTVYMELSSLRSEDTAVYYCARDAHQYYDFWSGYYSGTYYYGMDVWGQGTTVTVSS

SEQ ID NO: 274 - protein, 120 aa, Artificial Sequence
  Description of Artificial Sequence Synthetic polypeptide
QVQLVQSGAEVKKPGASVKVSCKASGGTFSNSIINWVRQAPGQGLEWMGWMNPNSGNTNYAQKFQGRVTMTRDTSTSTVYMELSSLRSEDTAVYYCAREQWPSYWYFDLWGRGTLVTVSS

SEQ ID NO: 275 - protein, 120 aa, Artificial Sequence
  Description of Artificial Sequence Synthetic polypeptide
QVQLVQSGAEVKKPGASVKVSCKASGGTFSTHDINWVRQAPGQGLEWMGVINPSGGSAIYAQKFQGRVTMTRDTSTSTVYMELSSLRSEDTAVYYCARDRGYSYGYFDYWGQGTLVTVSS

SEQ ID NO: 276 - protein, 124 aa, Artificial Sequence
  Description of Artificial Sequence Synthetic polypeptide
QVQLVQSGAEVKKPGASVKVSCKASGNTFIGYYVHWVRQAPGQGLEWVGIINPNGGSISYAQKFQGRVTMTRDTSTSTVYMELSSLRSEDTAVYYCARGSGDPNYYYYYGLDVWGQGTTVTVSS

SEQ ID NO: 277 - protein, 117 aa, Artificial Sequence
  Description of Artificial Sequence Synthetic polypeptide
QVQLVQSGAEVKKPGASVKVSCKASGYTLSYYYMHWVRQAPGQGLEWMGMIGPSDGSTSYAQRFQGRVTMTRDTSTGTVYMELSSLRSEDTAVYYCARDTGDHFDYWGQGTLVTVSS

SEQ ID NO: 278 - protein, 116 aa, Artificial Sequence
  Description of Artificial Sequence Synthetic polypeptide
QVQLVQSGAEVKKPGASVKVSCKASGYTFTGYYMHWVRQAPGQGLEWMGIIGPSDGSTTYAQKFQGRVTMTRDTSTSTVYMELSSLRSEDTAVYYCARAENGMDVWGQGTTVTVSS

SEQ ID NO: 279 - protein, 117 aa, Artificial Sequence
  Description of Artificial Sequence Synthetic polypeptide
QVQLVQSGAEVKKPGASVKVSCKASGYTFTGYYVHWVRQAPGQGLEWMGIIAPSDGSTNYAQKFQGRVTMTRDTSTSTVYMELSSLRSEDTAVYYCARDPGGYMDVWGKGTTVTVSS

SEQ ID NO: 280 - protein, 116 aa, Artificial Sequence
  Description of Artificial Sequence Synthetic polypeptide
QVQLVQSGAEVKKPGASVKVSCKASGYTFTGYYLHWVRQAPGQGLEWMGMIGPSDGSTSYAQKFQGRVTMTRDTSTSTVYMELSSLRSEDTAVYYCARDGDAFDIWGQGTMVTVSS

SEQ ID NO: 281 - protein, 117 aa, Artificial Sequence
  Description of Artificial Sequence Synthetic polypeptide
QVQLVQSGAEVKKPGSSVKVSCKASGYTFTGYYMHWVRQAPGQGLEWMGRISPSDGSTTYAPKFQGRVTITADESTSTAYMELSSLRSEDTAVYYCARDMGDAFDIWGQGTTVTVSS

SEQ ID NO: 282 - protein, 116 aa, Artificial Sequence
  Description of Artificial Sequence Synthetic polypeptide
QVQLVQSGAEVKKPGASVKVSCKASGYTFTGYYMHWVRQAPGQGLEWMGMIGPSDGSTSYAQRFQGRVTMTRDTSTSTVYMELSSLRSEDTAVYYCAREEDGMDVWGQGTTVTVSS

SEQ ID NO: 283 - protein, 124 aa, Artificial Sequence
  Description of Artificial Sequence Synthetic polypeptide
QVQLVQSGAEVKKPGSSVKVSCKASGGTFNNFAISWVRQAPGQGLEWMGGIIPIFDATNYAQKFQGRVTFTADESTSTAYMELSSLRSEDTAVYYCARGEYSSGFFFVGWFDLWGRGTQVTVSS

SEQ ID NO: 284 - protein, 118 aa, Artificial Sequence
  Description of Artificial Sequence Synthetic polypeptide
QVQLVQSGAEVKKPGASVKVSCKASGYNFTGYYMHWVRQAPGQGLEWMGIIAPSDGSTNYAQKFQGRVTMTRDTSTSTVYMELSSLRSEDTAVYYCARETGDDAFDIWGQGTMVTVSS

SEQ ID NO: 285 - protein, 107 aa, Artificial Sequence
  Description of Artificial Sequence Synthetic polypeptide
DIQMTQSPSSLSASVGDRVTITCRASQGISNYLAWYQQKPGKAPKLLIYAASSLQGGVPSRFSGSGSGTDFTLTISSLQPEDFATYYCQQYFTTPYTFGQGTKLEIK

SEQ ID NO: 286 - protein, 107 aa, Artificial Sequence
  Description of Artificial Sequence Synthetic polypeptide
DIQMTQSPSSLSASVGDRVTITCRASQSISRWLAWYQQKPGKAPKLLIFDASRLQSGVPSRFSGSGSGTDFTLTISSLQPEDFATYYCQQAEAFPYTFGQGTKVEIK

SEQ ID NO: 287 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIK

SEQ ID NO: 288 - protein, 107 aa, Artificial Sequence
  Description of Artificial Sequence Synthetic polypeptide
DIQMTQSPSSLSASVGDRVTITCRASQSISNYLNWYQQKPGKAPKLLIYKASSLESGVPSRFSGSGSGTDFTLTISSLQPEDFATYYCQQSYIIPYTFGQGTKLEIK

SEQ ID NO: 289 - protein, 107 aa, Artificial Sequence
  Description of Artificial Sequence Synthetic polypeptide
DIQMTQSPSSLSASVGDRVTITCRASQSISNYLNWYQQKPGKAPKLLIYAASSLQSGVPSRFSGSGSGTDFTLTISSLQPEDFATYYCHQTYSTPLTFGQGTKVEIK

SEQ ID NO: 290 - protein, 107 aa, Artificial Sequence
  Description of Artificial Sequence Synthetic polypeptide
DIQMTQSPSSLSASVGDRVTITCRASQGISNYLAWYQQKPGKAPKLLIYSASNLQSGVPSRFSGSGSGTDFTLTISSLQPEDFATYYCQQAYSFPWTFGQGTKVEIK

SEQ ID NO: 291 - protein, 107 aa, Artificial Sequence
  Description of Artificial Sequence Synthetic polypeptide
DIQMTQSPSSLSASVGDRVTITCRASQNISSYLNWYQQKPGKAPKLLIYAASSLQSGVPSRFSGSGSGTDFTLTISSLQPEDFATYYCQQGYSTPLTFGQGTRLEIK

SEQ ID NO: 292 - protein, 107 aa, Artificial Sequence
  Description of Artificial Sequence Synthetic polypeptide
DIQMTQSPSSLSASVGDRVTITCRASQDISRYLAWYQQKPGKAPKLLIYDASNLETGVPSRFSGSGSGTDFTLTISSLQPEDFATYYCQQANSFPRTFGQGTKVEIK

SEQ ID NO: 293 - protein, 107 aa, Artificial Sequence
  Description of Artificial Sequence Synthetic polypeptide
DIQMTQSPSSLSASVGDRVTITCQASQDISNYLNWYQQKPGKAPKLLIYAASNLQSGVPSRFSGSGSGTDFTLTISSLQPEDFATYYCQQANSLPYTFGQGTKVEIK

SEQ ID NO: 294 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASTLQNGVPSRFSGSGSGTDFTLTISSLQPEDFATYYCQQSYSTPFTFGPGTKVDIK

SEQ ID NO: 295 - protein, 107 aa, Artificial Sequence
  Description of Artificial Sequence Synthetic polypeptide
DIQMTQSPSSLSASVGDRVTITCRASQRISSYLNWYQQKPGKAPKLLIYSASTLQSGVPSRFSGSGSGTDFTLTISSLQPEDFATYYCQQSYSTPFTFGPGTKVDIK

SEQ ID NO: 296 - protein, 106 aa, Artificial Sequence
  Description of Artificial Sequence Synthetic polypeptide
DIQMTQSPSSLSASVGDRVTITCRASQSISSYLAWYQQKPGKAPKLLIYDASKLETGVPSRFSGSGSGTDFTLTISSLQPEDFATYYCQQSYGVPTFGQGTKLEIK

SEQ ID NO: 297 - protein, 107 aa, Artificial Sequence
  Description of Artificial Sequence Synthetic polypeptide
DIQMTQSPSSLSASVGDRVTITCRASQGISSWLAWYQQKPGKAPKLLIYDASNLETGVPSRFSGSGSGTDFTLTISSLQPEDFATYYCQQSYSTPLTFGGGTKVEIK

SEQ ID NO: 298 - protein, 107 aa, Artificial Sequence
  Description of Artificial Sequence Synthetic polypeptide
DIQMTQSPSSLSASVGDRVTITCRASQGISTYLAWYQQKPGKAPKLLIYDASSLQSGVPSRFSGSGSGTDFTLTISSLQPEDFATYYCQQYYSYPWTFGQGTRLEIK

SEQ ID NO: 299 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQTLKTPLSFGGGTKVEIK

SEQ ID NO: 300 - protein, 18 aa, Artificial Sequence
  Description of Artificial Sequence Synthetic peptide
CARDGYDFWSGYTSDDYW

SEQ ID NO: 301 - protein, 9 aa, Artificial Sequence
  Description of Artificial Sequence Synthetic peptide
CASDYGDYR

SEQ ID NO: 302 - protein, 20 aa, Artificial Sequence
  Description of Artificial Sequence Synthetic peptide
CARDLMTTVVTPGDYGMDVW

SEQ ID NO: 303 - protein, 14 aa, Artificial Sequence
  Description of Artificial Sequence Synthetic peptide
CARQDGGAFAFDIW

SEQ ID NO: 304 - protein, 14 aa, Artificial Sequence [sequence omitted]

SEQ ID NO: 305 - protein, 19 aa, Artificial Sequence
  Description of Artificial Sequence Synthetic peptide
CARALIFGVPLLPYGMDVW

SEQ ID NO: 306 - protein, 20 aa, Artificial Sequence
  Description of Artificial Sequence Synthetic peptide
CAKDLATVGEPYYYYGMDVW

SEQ ID NO: 307 - protein, 20 aa, Artificial Sequence
  Description of Artificial Sequence Synthetic peptide
CARLWFGELHYYYYYGMDVW

SEQ ID NO: 308 - protein, 10 aa, Artificial Sequence
  Description of Artificial Sequence Synthetic peptide
CHHYGRSHTF

SEQ ID NO: 309 - protein, 11 aa, Artificial Sequence
  Description of Artificial Sequence Synthetic peptide
CQQANAFPPTF

SEQ ID NO: 310 - protein, 11 aa, Artificial Sequence [sequence omitted]

SEQ ID NO: 311 - protein, 11 aa, Artificial Sequence
  Description of Artificial Sequence Synthetic peptide
CQQSYSTPPTF

SEQ ID NO: 312 - protein, 11 aa, Artificial Sequence [sequence omitted]

SEQ ID NO: 313 - protein, 11 aa, Artificial Sequence [sequence omitted]

SEQ ID NO: 314 - protein, 11 aa, Artificial Sequence
  Description of Artificial Sequence Synthetic peptide
CMQGSHWPPSF

SEQ ID NO: 315 - protein, 107 aa, Artificial Sequence
  Description of Artificial Sequence Synthetic polypeptide
EIVMTQSPATLSVSPGERATLSCRASQSVSSSNLAWYQQKPGQAPRLLIYGASTRATGIPARFSGSGSGTEFTLTISSLQSEDFAVYYCHHYGRSHTFGQGTKVEIK

SEQ ID NO: 316 - protein, 107 aa, Artificial Sequence
  Description of Artificial Sequence Synthetic polypeptide
DIQMTQSPSSLSASVGDRVTITCRASQDIRNDLGWYQQKPGKAPKLLIYAASSLQSGVPSRFSGSGSGTDFTLTISSLQPEDFATYYCQQANAFPPTFGQGTKVEIK

SEQ ID NO: 317 - protein, 113 aa, Artificial Sequence
  Description of Artificial Sequence Synthetic polypeptide
DIVMTQSPDSLAVSLGERATINCKSSQSVFYSSNNKNQLAWYQQKPGQPPKLLIYWASTRESGVPDRFSGSGSGTDFTLTISSLQAEDVAVYYCQQYYSIPLTFGQGTKLEIK

SEQ ID NO: 318 - protein, 107 aa, Artificial Sequence
  Description of Artificial Sequence Synthetic polypeptide
DIQMTQSPSSLSASVGDRVTITCQASQDIFKYLNWYQQKPGKAPKLLIYAASTLQSGVPSRFSGSGSGTDFTLTISSLQPEDFATYYCQQSYSTPPTFGQGTRLEIK

SEQ ID NO: 319 - protein, 107 aa, Artificial Sequence
  Description of Artificial Sequence Synthetic polypeptide
DIQMTQSPSSLSASVGDRVTITCRASQSISTWLAWYQQKPGKAPKLLIYYASSLQSGVPSRFSGSGSGTDFTLTISSLQPEDFATYYCQQSYSFPYTFGQGTKVEIK

SEQ ID NO: 320 - protein, 112 aa, Artificial Sequence
  Description of Artificial Sequence Synthetic polypeptide
DIVMTQSPLSLPVTPGEPASISCSSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 321 - protein, 107 aa, Artificial Sequence
  Description of Artificial Sequence Synthetic polypeptide
DIQMTQSPSSLSASVGDRVTITCQASQDISNYLNWYQQKPGKAPKLLIYSASNLRSGVPSRFSGSGSGTDFTLTISSLQPEDFATYYCQQGNTFPLTFGQGTKVEIK

SEQ ID NO: 322 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGSHWPPSFGQGTRLEIK

SEQ ID NO: 323 - protein, 122 aa, Artificial Sequence
  Description of Artificial Sequence Synthetic polypeptide
QVQLVQSGAEVKKPGASVKVSCKASGGTFSNYGISWVRQAPGQGLEWMGIINPGGSTSYAQKFQGRVTMTRDTSTSTVYMELSSLRSEDTAVYYCARDGYDFWSGYTSDDYWGQGTLVTVSS

SEQ ID NO: 324 - protein, 114 aa, Artificial Sequence
  Description of Artificial Sequence Synthetic polypeptide
EVQLLESGGGLVQPGGSLRLSCAASGFTFSSYAMHWVRQAPGKGLEWVSGISGSGGSTYYADSVKGRFTISRDNSKNTLYLQMNSLRAEDTAVYYCASDYGDYRGQGTLVTVSS

SEQ ID NO: 325 - protein, 125 aa, Artificial Sequence
  Description of Artificial Sequence Synthetic polypeptide
QVQLVQSGAEVKKPGASVKVSCKASGYTFSNYYIHWVRQAPGQGLEWMGWLNPNSGNTGYAQRFQGRVTMTRDTSTSTVYMELSSLRSEDTAVYYCARDLMTTVVTPGDYGMDVWGQGTTVTVSS

SEQ ID NO: 326 - protein, 119 aa, Artificial Sequence
  Description of Artificial Sequence Synthetic polypeptide
QVQLVQSGAEVKKPGASMKVSCKASGYTFTTDGISWVRQAPGQGLEWMGRIYPHSGYTEYAKKFKGRVTMTRDTSTSTVYMELSSLRSEDTAVYYCARQDGGAFAFDIWGQGTMVTVSS

SEQ ID NO: 327 - protein, 119 aa, Artificial Sequence
  Description of Artificial Sequence Synthetic polypeptide
QVQLVQSGAEVKKPGASVKVSCKASGYTFTSQYMHWVRQAPGQGLEWMGWISPNNGDTNYAQKFQGRVTMTRDTSTSTVYMELSSLRSEDTAVYYCARELGYYYGMDVWGQGTTVTVSS

SEQ ID NO: 328 - protein, 124 aa, Artificial Sequence
  Description of Artificial Sequence Synthetic polypeptide
QVQLVQSGAEVKKPGSSVKVSCKASRYTFTSYDINWVRQAPGQGLEWMGRIIPMLNIANYAPKFQGRVTITADESTSTAYMELSSLRSEDTAVYYCARALIFGVPLLPYGMDVWGQGTTVTVSS

SEQ ID NO: 329 - protein, 125 aa, Artificial Sequence
  Description of Artificial Sequence Synthetic polypeptide
EVQLLQSGGGLVQPGGSLRLSCAASGFTFSSSWMHWVRQAPGKGLEWVSFISTSSGYIYYADSVKGRFTISRDNSKNTLYLQMNSLRAEDTAVYYCAKDLATVGEPYYYYGMDVWGQGTTVTVSS

SEQ ID NO: 330 - protein, 125 aa, Artificial Sequence
  Description of Artificial Sequence Synthetic polypeptide
QVQLVQSGAEVKKPGSSVKVSCKASGDTFNTYALSWVRQAPGQGLEWMGWMNPNSGNAGYAQKFQGRVTITADESTSTAYMELSSLRSEDTAVYYCARLWFGELHYYYYYGMDVWGQGTMVTVSS

SEQ ID NO: 331 - protein, 107 aa, Artificial Sequence
  Description of Artificial Sequence Synthetic polypeptide
DIQMTQSPSSLSASVGDRVTITCRASQSISTWLAWYQQKPGKAPKLLIYAASSLRSGVPSRFSGSGSGTDFTLTISSLQPEDFATYYCQQSYNTPYTFGQGTKLEIK

SEQ ID NO: 332 - protein, 107 aa, Artificial Sequence
  Description of Artificial Sequence Synthetic polypeptide
DIQMTQSPSSLSASVGDRVTITCRASQSISRWLAWYQQKPGKAPKLLIYAASTVQSGVPSRFSGSGSGTDFTLTISSLQPEDFATYYCQQSYSTPYTFGQGTKLEIK

SEQ ID NO: 333 - protein, 107 aa, Artificial Sequence
  Description of Artificial Sequence Synthetic polypeptide
DIQMTQSPSSLSASVGDRVTITCRASQDISRWLAWYQQKPGKAPKLLIYAASRLQAGVPSRFSGSGSGTDFTLTISSLQPEDFATYYCQQSYSTPYSFGQGTKLEIK

SEQ ID NO: 334 - protein, 107 aa, Artificial Sequence
  Description of Artificial Sequence Synthetic polypeptide
DIQMTQSPSSLSASVGDRVTITCRASQTISSWLAWYQQKPGKAPKLLIYAASSLQSGVPSRFSGSGSGTDFTLTISSLQPEDFATYYCQQSYSTPFTFGPGTKVDIK

SEQ ID NO: 335 - protein, 107 aa, Artificial Sequence
  Description of Artificial Sequence Synthetic polypeptide
DIQMTQSPSSLSASVGDRVTITCRASQTISSWLAWYQQKPGKAPKLLIYAASSLQSGVPSRFSGSGSGTDFTLTISSLQPEDFATYYCQQSYGVPYTFGQGTKVEIK

SEQ ID NO: 336 - protein, 107 aa, Artificial Sequence
  Description of Artificial Sequence Synthetic polypeptide
DIQMTQSPSSLSASVGDRVTITCRASQSISNWLAWYQQKPGKAPKLLIYAASSLQSGVPSRFSGSGSGTDFTLTISSLQPEDFATYYCQQSYSAPYTFGPGTKVDIK

SEQ ID NO: 337 - protein, 107 aa, Artificial Sequence
  Description of Artificial Sequence Synthetic polypeptide
DIQMTQSPSSLSASVGDRVTITCRASQSVGNWLAWYQQKPGKAPKLLIYGASSLQTGVPSRFSGSGSGTDFTLTISSLQPEDFATYYCQQSYSAPYTFGQGTKVEIK

SEQ ID NO: 338 - protein, 107 aa, Artificial Sequence
  Description of Artificial Sequence Synthetic polypeptide
DIQMTQSPSSLSASVGDRVTITCRASQNIGNWLAWYQQKPGKAPKLLIYAASTLQTGVPSRFSGSGSGTDFTLTISSLQPEDFATYYCQQSYSAPYSFGQGTKLEIK

SEQ ID NO: 339 - protein, 107 aa, Artificial Sequence
  Description of Artificial Sequence Synthetic polypeptide
DIQMTQSPSSLSASVGDRVTITCRASQSISRWLAWYQQKPGKAPKLLIYAASSLQSGVPSRFSGSGSGTDFTLTISSLQPEDFATYYCQQSYSTPYTFGQGTKLEIK

SEQ ID NO: 340 - protein, 107 aa, Artificial Sequence
  Description of Artificial Sequence Synthetic polypeptide
DIQMTQSPSSLSASVGDRVTITCRASQSISSWLAWYQQKPGKAPKLLIYGASSLQSGVPSRFSGSGSGTDFTLTISSLQPEDFATYYCQQSYSVPYSFGQGTKLEIK

SEQ ID NO: 341 - protein, 107 aa, Artificial Sequence
  Description of Artificial Sequence Synthetic polypeptide
DIQMTQSPSSLSASVGDRVTITCRASQSISKWLAWYQQKPGKAPKLLIYAASSLQSGVPSRFSGSGSGTDFTLTISSLQPEDFATYYCQQSYSAPYTFGQGTKVEIK

SEQ ID NO: 342 - protein, 107 aa, Artificial Sequence
  Description of Artificial Sequence Synthetic polypeptide
DIQMTQSPSSLSASVGDRVTITCRASQGISNYLAWYQQKPGKAPKLLIYAASTLQSGVPSRFSGSGSGTDFTLTISSLQPEDFATYYCQQSYSVPYSFGQGTKLEIK

SEQ ID NO: 343 - protein, 107 aa, Artificial Sequence
  Description of Artificial Sequence Synthetic polypeptide
DIQMTQSPSSLSASVGDRVTITCRASQTISNYLNWYQQKPGKAPKLLIYAASNLQSGVPSRFSGSGSGTDFTLTISSLQPEDFATYYCQQSYSTPQTFGQGTKVEIK

SEQ ID NO: 344 - protein, 107 aa, Artificial Sequence
  Description of Artificial Sequence Synthetic polypeptide
DIQMTQSPSSLSASVGDRVTITCRASRDIGRAVGWYQQKPGKAPKLLIYAASSLQSGVPSRFSGSGSGTDFTLTISSLQPEDFATYYCQQLDSYPFTFGPGTKVDIK

SEQ ID NO: 345 - protein, 107 aa, Artificial Sequence
  Description of Artificial Sequence Synthetic polypeptide
DIQMTQSPSSLSASVGDRVTITCRASQSISSWLAWYQQKPGKAPKLLIYAASTLQSGVPSRFSGSGSGTDFTLTISSLQPEDFATYYCQQSYSSPYTFGPGTKVDIK

SEQ ID NO: 346 - protein, 107 aa, Artificial Sequence
  Description of Artificial Sequence Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQSYSTPLTFGGGTKLEIK

SEQ ID NO: 347 - protein, 107 aa, Artificial Sequence
  Description of Artificial Sequence Synthetic polypeptide
DIQMTQSPSSLSASVGDRVTITCRASQSIGRWLAWYQQKPGKAPKLLIYAASSLQSGVPSRFSGSGSGTDFTLTISSLQPEDFATYYCQQSYSTPYSFGQGTKVEIK

SEQ ID NO: 348 - protein, 107 aa, Artificial Sequence
  Description of Artificial Sequence Synthetic polypeptide
DIQMTQSPSSLSASVGDRVTITCRASQSISTWLAWYQQKPGKAPKLLIYAASTLQSGVPSRFSGSGSGTDFTLTISSLQPEDFATYYCQQSYSTPYTFAQGTKLEIK

SEQ ID NO: 349 - protein, 107 aa, Artificial Sequence
  Description of Artificial Sequence Synthetic polypeptide
DIQMTQSPSSLSASVGDRVTITCQASQDISNYLNWYQQKPGKAPKLLIYGASRLQSGVPSRFSGSGSGTDFTLTISSLQPEDFATYYCQQSYSTPYTFGQGTKLEIK

SEQ ID NO: 350 - protein, 107 aa, Artificial Sequence
  Description of Artificial Sequence Synthetic polypeptide
DIQMTQSPSSLSASVGDRVTITCRASQSISSWLAWYQQKPGKAPKLLIYAASTLQSGVPSRFSGSGSGTDFTLTISSLQPEDFATYYCQQSYSTPFTFGPGTKVDIK

SEQ ID NO: 351 - protein, 106 aa, Artificial Sequence
  Description of Artificial Sequence Synthetic polypeptide
DIQMTQSPSSLSASVGDRVTITCRASQSVSNWLAWYQQKPGKAPKLLIYAASSLQSGVPSRFSGSGSGTDFTLTISSLQPEDFATYYCQQSYSTPTFGQGTKLEIK

SEQ ID NO: 352 - protein, 107 aa, Artificial Sequence
  Description of Artificial Sequence Synthetic polypeptide
DIQMTQSPSSLSASVGDRVTITCQASQDISNYLNWYQQKPGKAPKLLIYAASTLQSGVPSRFSGSGSGTDFTLTISSLQPEDFATYYCQQTYAIPLTFGGGTKVEIK

SEQ ID NO: 353 - protein, 107 aa, Artificial Sequence
  Description of Artificial Sequence Synthetic polypeptide
DIQMTQSPSSLSASVGDRVTITCQASQDIGSWLAWYQQKPGKAPKLLIYATSSLQSGVPSRFSGSGSGTDFTLTISSLQPEDFATYYCQQSYSTPYTFGQGTKLEIK

SEQ ID NO: 354 - protein, 107 aa, Artificial Sequence
  Description of Artificial Sequence Synthetic polypeptide
DIQMTQSPSSLSASVGDRVTITCRASQGISRWLAWYQQKPGKAPKLLIYAASTLQPGVPSRFSGSGSGTDFTLTISSLQPEDFATYYCQQSYIAPFTFGPGTKVDIK

SEQ ID NO: 355 - protein, 107 aa, Artificial Sequence
  Description of Artificial Sequence Synthetic polypeptide
DIQMTQSPSSLSASVGDRVTITCRASQGISNYLAWYQQKPGKAPKLLIYAASRLESGVPSRFSGSGSGTDFTLTISSLQPEDFATYYCQQSYSIPLTFGGGTKVEIK

SEQ ID NO: 356 - protein, 106 aa, Artificial Sequence
  Description of Artificial Sequence Synthetic polypeptide
DIQMTQSPSSLSASVGDRVTITCRASQSISSYLNWYQQKPGKAPKLLIYGVSSLQSGVPSRFSGSGSGTDFTLTISSLQPEDFATYYCQQSYSNPTFGQGTKVEIK

SEQ ID NO: 357 - protein, 107 aa, Artificial Sequence
  Description of Artificial Sequence Synthetic polypeptide
DIQMTQSPSSLSASVGDRVTITCRASQSISSWVAWYQQKPGKAPKLLIYGASNLESGVPSRFSGSGSGTDFTLTISSLQPEDFATYYCQQSYSTPYSFGQGTKLEIK

SEQ ID NO: 358 - protein, 107 aa, Artificial Sequence
  Description of Artificial Sequence Synthetic polypeptide
DIQMTQSPSSLSASVGDRVTITCRASQGISNYLAWYQQKPGKAPKLLIYAASSLQSGVPSRFSGSGSGTDFTLTISSLQPEDFATYYCQQSYSDQWTFGQGTKVEIK

SEQ ID NO: 359 - protein, 107 aa, Artificial Sequence
  Description of Artificial Sequence Synthetic polypeptide
DIQMTQSPSSLSASVGDRVTITCRASQSISRWLAWYQQKPGKAPKLLIYAASSLQSGVPSRFSGSGSGTDFTLTISSLQPEDFATYYCQQSYLPPYSFGQGTKVEIK

SEQ ID NO: 360 - protein, 107 aa, Artificial Sequence
  Description of Artificial Sequence Synthetic polypeptide
DIQMTQSPSSLSASVGDRVTITCRASQSISNWLAWYQQKPGKAPKLLIYAASSLQSGVPSRFSGSGSGTYFTLTISSLQPEDFATYYCQQSYSSPYTFGQGTKLEIK

SEQ ID NO: 361 - protein, 107 aa, Artificial Sequence
  Description of Artificial Sequence Synthetic polypeptide
DIQMTQSPSSLSASVGDRVTITCRASQSISHYLNWYQQKPGKAPKLLIYGASSLQSGVPSRFSGSGSGTDFTLTISSLQPEDFATYYCQQSYTTPWTFGQGTRLEIK

SEQ ID NO: 362 - protein, 107 aa, Artificial Sequence
  Description of Artificial Sequence Synthetic polypeptide
DIQMTQSPSSLSASVGDRVTITCRASQSISSWLAWYQQKPGKAPKLLIYAASTLQSGVPSRFSGSGSGTDFTLTISSLQPEDFATYYCQQSYLPPYSFGQGTKLEIK

SEQ ID NO: 363 - protein, 105 aa, Artificial Sequence
  Description of Artificial Sequence Synthetic polypeptide
DIQMTQSPSSLSASVGDRVTITCQASQDISNYLNWYQQKPGKAPKLLIYGASRLQSGVPSRFSGSGSGTDFTLTISSLQPEDFATYYCQEGITYTFGQGTKVEIK

SEQ ID NO: 364 - protein, 107 aa, Artificial Sequence
  Description of Artificial Sequence Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIK

SEQ ID NO: 365 - protein, 107 aa, Artificial Sequence
  Description of Artificial Sequence Synthetic polypeptide
EIVMTQSPATLSVSPGERATLSCRASQSVSRNLAWYQQKPGQAPRLLIYGASTRATGIPARFSGSGSGTEFTLTISSLQSEDFAVYYCQHYGYSPVTFGQGTKLEIK

SEQ ID NO: 366 - protein, 115 aa, Artificial Sequence
  Description of Artificial Sequence Synthetic polypeptide
QVQLVQSGAEVKKPGASVKVSCKASGGTFSSATISWVRQAPGQGLEWMGWIYPNSGGTVYAQKFQGRVTMTRDTSTSTVYMELSSLRSEDTAVYYCAATEWLGVWGQGTTVTVSS

SEQ ID NO: 367 - protein, 115 aa, Artificial Sequence
  Description of Artificial Sequence Synthetic polypeptide
EVQLLQSGAEVKKPGSSVKVSCKASGGTFSSYAISWVRQAPGQGLEWMGWINPNSGGTISAPNFQGRVTMTRDTSTSTVYMELSSLRSEDTAVYYCARANWLDYWGQGTLVTVSS

SEQ ID NO: 368 - protein, 115 aa, Artificial Sequence
  Description of Artificial Sequence Synthetic polypeptide
EVQLLESGAEVKKPGASVKVSCKASGYTFTTYDLAWVRQAPGQGLEWMGWINPNSGGTNYAQKFQGRVTMTRDTSTSTVYMELSSLRSEDTAVYYCARANWLDYWGQGTLVTVSS

SEQ ID NO: 369 - protein, 115 aa, Artificial Sequence
  Description of Artificial Sequence Synthetic polypeptide
QVQLVQSGAEVKKPGASVKVSCKSSGYSFDSYVVNWVRQAPGQGLEWMGWINPNSGGTNYAQKFQGRVTMTRDTSTSTVYMELSSLRSEDTAVYYCARDWVLDYWGQGTLVTVSS

SEQ ID NO: 370 - protein, 115 aa, Artificial Sequence
  Description of Artificial Sequence Synthetic polypeptide
QVQLVQSGAEVKKPGASVKVSCKASGYTFTSYGISWVRQAPGQGLEWMGWMNPNSGGTNYAQKFQGRVTMTRDTSTSTVYMELSSLRSEDTAVYYCARGEWLDYWGQGTLVTVSS

SEQ ID NO: 371 - protein, 115 aa, Artificial Sequence
  Description of Artificial Sequence Synthetic polypeptide
QVQLVQSGAEVKKPGASVKVSCKASGYTFTSYGISWVRQAPGQGLEWMGWINPNSGGTNYAQKFQGRVTMTRDTSTSTVYMELSSLRSEDTAVYYCARGWELGYWGQGTLVTVSS

SEQ ID NO: 372 - protein, 116 aa, Artificial Sequence
  Description of Artificial Sequence Synthetic polypeptide
QVQLVQSGAEVKKPGASVKVSCKASGYTFTRYTINWVRQAPGQGLEWMGWINPNSGGTNYAQKFQGRVTMTRDTSTSTVYMELSSLRSEDTAVYYCARDFVGYDDWGQGTLVTVSS

SEQ ID NO: 373 - protein, 116 aa, Artificial Sequence
  Description of Artificial Sequence Synthetic polypeptide
QVQLVQSGAEVKKPGASVKVSCKASGYTFTSYGITWVRQAPGQGLEWMGWINPNSGGTNYAQKFQGRVTMTRDTSTSTVYMELSSLRSEDTAVYYCARDYGDLDYWGQGTLVTVSS

SEQ ID NO: 374 - protein, 116 aa, Artificial Sequence
  Description of Artificial Sequence Synthetic polypeptide
QVQLVQSGAEVKKPGASVKVSCKASGGTFSNYILSWVRQAPGQGLEWMGWINPDSGGTNYAQKFQGRVTMTRDTSTSTVYMELSSLRSEDTAVYYCARGSYGMDVWGQGTTVTVSS

SEQ ID NO: 375 - protein, 117 aa, Artificial Sequence
  Description of Artificial Sequence Synthetic polypeptide
QVQLVQSGAEVKKPGASVKVSCKASGYSFTRYNMHWVRQAPGQGLEWMGWINPNSGGTNYAQKFQGRVTMTRDTSTSTVYMELSSLRSEDTAVYYCARDGYSGLDVWGKGTTVTVSS

SEQ ID NO: 376 - protein, 117 aa, Artificial Sequence
  Description of Artificial Sequence Synthetic polypeptide
QVQLVQSGAEVKKPGASVKVSCKASGGTFSSYAISWVRQAPGQGLEWMGWINPNNGGTNYAQKFQGRVTMTRDTSTSTVYMELSSLRSEDTAVYYCARDSGVGMDVWGQGTTVTVSS

SEQ ID NO: 377 - protein, 118 aa, Artificial Sequence
  Description of Artificial Sequence Synthetic polypeptide
QVQLVQSGAEVKKPGASVKVSCKASGGTFNNYAFSWVRQAPGQGLEWMGWINPNSGGTNYAQKFQGRVTMTRDTSTSTVYMELSSLRSEDTAVYYCARDGVAVASDYWGQGTLVTVSS

SEQ ID NO: 378 - protein, 118 aa, Artificial Sequence
  Description of Artificial Sequence Synthetic polypeptide
QVQLVQSGAEVKKPGASVKVSCKASGYTFSSYNMHWVRQAPGQGLEWMGWINGNTGGTNYAQKFQGRVTMTRDTSTSTVYMELSSLRSEDTAVYYCARGVNVDDFDYWGQGTLVTVSS

SEQ ID NO: 379 - protein, 120 aa, Artificial Sequence
  Description of Artificial Sequence Synthetic polypeptide
QVQLVQSGAEVKKPGASVKVSCKASGGTFSSYAFSWVRQAPGQGLEWMGWINPDTGYTRYAQKFQGRVTMTRDTSTSTVYMELSSLRSEDTAVYYCARGDYTGNWYFDLWGRGTLVTVSS

SEQ ID NO: 380 - protein, 115 aa, Artificial Sequence
  Description of Artificial Sequence Synthetic polypeptide
EVQLLESGAEVKKPGASVKVSCKASGYTFTSYGISWVRQAPGQGLEWMGWINPYSGGTNYAQKLQGRVTMTRDTSTSTVYMELSSLRSEDTAVYYCARANWLDYWGQGTLVTVSS

SEQ ID NO: 381 - protein, 122 aa, Artificial Sequence
  Description of Artificial Sequence Synthetic polypeptide
QVQLVQSGAEVKKPGASVKVSCKASGYTFTSYGISWVRQAPGQGLEWMGWISAYNGYTNYAQNLQGRVTMTRDTSTSTVYMELSSLRSEDTAVYYCARDQFYGGNSGGHDYWGQGTLVTVSS

SEQ ID NO: 382 - protein, 113 aa, Artificial Sequence
  Description of Artificial Sequence Synthetic polypeptide
QVQLVQSGAEVKKPGASVKVSCKASGYTFTDYNMHWVRQAPGQGLEWMGWMNPNSGGTNYAQKFQGRVTMTRDTSTSTVYMELSSLRSEDTAVYYCAREEDYWGQGTLVTVSS

SEQ ID NO: 383 - protein, 115 aa, Artificial Sequence
  Description of Artificial Sequence Synthetic polypeptide
QVQLVQSGAEVKKPGASVKVSCKASGYTFTRYTINWVRQAPGQGLEWMGWINPNSGGANYAQKFQGRVTMTRDTSTSTVYMELSSLRSEDTAVYYCARGDWFDPWGQGTLVTVSS

SEQ ID NO: 384 - protein, 115 aa, Artificial Sequence
  Description of Artificial Sequence Synthetic polypeptide
QVQLVQSGAEVKKPGASVKVSCKASGYTFTSYLMHWVRQAPGQGLEWMGWISPNSGGTNYAQKFQGRVTMTRDTSTSTVYMELSSLRSEDTAVYYCARGDWFDPWGQGTLVTVSS

SEQ ID NO: 385 - protein, 115 aa, Artificial Sequence
  Description of Artificial Sequence Synthetic polypeptide
QVQLVQSGAEVKKPGASVKVSCKASGYTFSDYYVHWVRQAPGQGLEWMGWINPNSGGTNYAQKFQGRVTMTRDTSTSTVYMELSSLRSEDTAVYYCARGEWFDPWGQGTLVTVSS

SEQ ID NO: 386 - protein, 115 aa, Artificial Sequence
  Description of Artificial Sequence Synthetic polypeptide
QVQLVQSGAEVKKPGASVKVSCKASGYTFTTYYMHWVRQAPGQGLEWMGWINPNSGGTNYAQKFQGRVTMTRDTSTSTVYMELSSLRSEDTAVYYCARSDWFDPWGQGTLVTVSS

SEQ ID NO: 387 - protein, 117 aa, Artificial Sequence
  Description of Artificial Sequence Synthetic polypeptide
QVQLVQSGAEVKKPGASVKVSCKASGGTFSNYAINWVRQAPGQGLEWMGWISPYSGGTNYAQKFQGRVTMTRDTSTSTVYMELSSLRSEDTAVYYCARDSGSYFDYWGQGTLVTVSS

SEQ ID NO: 388 - protein, 117 aa, Artificial Sequence
  Description of Artificial Sequence Synthetic polypeptide
QVQLVQSGAEVKKPGASVKVSCKASGYTFTDYYMHWVRQAPGQGLEWMGWIYPNTGGTNYAQKFQGRVTMTRDTSTSTVYMELSSLRSEDTAVYYCARDYGGYVDYWGQGTLVTVSS

SEQ ID NO: 389 - protein, 117 aa, Artificial Sequence
  Description of Artificial Sequence Synthetic polypeptide
EVQLLESGAEVKKPGASVKVSCKASGYTFTSYAMNWVRQAPGQGLEWMGWMNPNSGGTKYAQKFQGRVTMTRDTSTSTVYMELSSLRSEDTAVYYCAREGPAALDVWGQGTLVTVSS

SEQ ID NO: 390 - protein, 117 aa, Artificial Sequence
  Description of Artificial Sequence Synthetic polypeptide
QVQLVQSGAEVKKPGASVKVSCKASGYTLTSHLIHWVRQAPGQGLEWMGWINPNSGGTNYAQKFQGRVTMTRDTSTSTVYMELSSLRSEDTAVYYCARERRSGMDVWGQGTTVTVSS

SEQ ID NO: 391 - protein, 117 aa, Artificial Sequence
  Description of Artificial Sequence Synthetic polypeptide
EVQLLESGAEVKKPGASVKVSCKASGYSFTDYIVHWVRQAPGQGLEWMGWINPYSGGTKYAQKFQGRVTMTRDTSTSTVYMELSSLRSEDTAVYYCARVLQEGMDVWGQGTLVTVSS

SEQ ID NO: 392 - protein, 117 aa, Artificial Sequence
  Description of Artificial Sequence Synthetic polypeptide
QVQLVQSGAEVKKPGASVKVSCKASGYTFSNFLINWVRQAPGQGLEWMGWINPNSGGTNYAQKFQGRVTMTRDTSTSTVYMELSSLRSEDTAVYYCASERELPFDIWGQGTMVTVSS

SEQ ID NO: 393 - protein, 118 aa, Artificial Sequence
  Description of Artificial Sequence Synthetic polypeptide
QVQLVQSGAEVKKPGASVKVSCKASGYTFTDYQMFWVRQAPGQGLEWMGWINPNSGGTNYAQKFQGRVTMTRDTSTSTVYMELSSLRSEDTAVYYCAKGGGGYGMDVWGQGTTVTVSS

SEQ ID NO: 394 - protein, 120 aa, Artificial Sequence
  Description of Artificial Sequence Synthetic polypeptide
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCAAMGIAVAGGMDVWGQGTLVTVSS

SEQ ID NO: 395 - protein, 115 aa, Artificial Sequence
  Description of Artificial Sequence Synthetic polypeptide
QVQLVQSGAEVKKPGASVKVSCKASGYTFTNYHMHWVRQAPGQGLEWMGWIHPDSGGTSYAQKFQGRVTMTRDTSTSTVYMELSSLRSEDTAVYYCARNWNLDYWGQGTLVTVSS

SEQ ID NO: 396 - protein, 116 aa, Artificial Sequence
  Description of Artificial Sequence Synthetic polypeptide
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCATYDDGMDVWGQGTTVTVSS

SEQ ID NO: 397 - protein, 117 aa, Artificial Sequence
  Description of Artificial Sequence Synthetic polypeptide
QVQLVQSGAEVKKPGASVKVSCKASGYTFTSYTVNWVRQAPGQGLEWMGWINPNSGGTKYAQNFQGRVTMTRDTSTSTVYMELSSLRSEDTAVYYCARGGGGALDYWGQGTLVTVSS

SEQ ID NO: 398 - protein, 118 aa, Artificial Sequence
  Description of Artificial Sequence Synthetic polypeptide
QVQLVQSGAEVKKPGASVKVSCKASGYTFTSYYMHWVRQAPGQGLEWMGMINPRDDTTDYARDFQGRVTMTRDTSTSTVYMELSSLRSEDTAVYYCALSGNYYGMDVWGQGTTVTVSS

SEQ ID NO: 399 - protein, 119 aa, Artificial Sequence
  Description of Artificial Sequence Synthetic polypeptide
QVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSS

SEQ ID NO: 400 - protein, 118 aa, Artificial Sequence
  Description of Artificial Sequence Synthetic polypeptide
QVQLVQSGAEVKKPGSSVKVSCKASGYTFTSQYMHWVRQAPGQGLEWMGRIIPLLGIVNYAQKFQGRVTITADESTSTAYMELSSLRSEDTAVYYCARDKNYYGMDVWGQGTTVTVSS

SEQ ID NO: 401 - protein, 11 aa, Artificial Sequence
  Description of Artificial Sequence Synthetic peptide
YSEHPTFTSQY

SEQ ID NO: 402 - protein, 6 aa, Artificial Sequence
  Description of Artificial Sequence Synthetic peptide
GSGGGG

SEQ ID NO: 403 - protein, 17 aa, Artificial Sequence
  Description of Artificial Sequence Synthetic peptide
GGGGSEPKSSDKTHTCP

SEQ ID NO: 404 - protein, 4 aa, Artificial Sequence
  Description of Artificial Sequence Synthetic peptide
GGGC

SEQ ID NO: 405 - protein, 15 aa, Artificial Sequence
  Description of Artificial Sequence Synthetic peptide
GGGGSEPKSSDKTHT

SEQ ID NO: 406 - protein, 20 aa, Artificial Sequence
  Description of Artificial Sequence Synthetic peptide
GGCGGGGSGSEPKSSDKTHT

SEQ ID NO: 407 - protein, 15 aa, Artificial Sequence
  Description of Artificial Sequence Synthetic peptide
GGCGGGGSGSGGGGS

SEQ ID NO: 408 - protein, 15 aa, Artificial Sequence
  Description of Artificial Sequence Synthetic peptide
GGCGSEPKSSDKTHT

SEQ ID NO: 409 - protein, 5 aa, Artificial Sequence
  Description of Artificial Sequence Synthetic peptide
GGCGS

SEQ ID NO: 410 - protein, 21 aa, Artificial Sequence
  Description of Artificial Sequence Synthetic peptide
GGGCGGGGSGSEPKSSDKTHT

SEQ ID NO: 411 - protein, 16 aa, Artificial Sequence
  Description of Artificial Sequence Synthetic peptide
GGGCGGGGSGSGGGGS

SEQ ID NO: 412 - protein, 16 aa, Artificial Sequence
  Description of Artificial Sequence Synthetic peptide
GGGCGSEPKSSDKTHT

SEQ ID NO: 413 - protein, 6 aa, Artificial Sequence
  Description of Artificial Sequence Synthetic peptide
GGGCGS

SEQ ID NO: 414 - protein, 9 aa, Artificial Sequence
  Description of Artificial Sequence Synthetic peptide
AAACSRMVI

SEQ ID NO: 415 - protein, 9 aa, Artificial Sequence
  Description of Artificial Sequence Synthetic peptide
LLFGYPVYV

SEQ ID NO: 416 - protein, 9 aa, Artificial Sequence
  Description of Artificial Sequence Synthetic peptide
GILGFVFTL

SEQ ID NO: 417 - protein, 9 aa, Artificial Sequence
  Description of Artificial Sequence Synthetic peptide
FLLTRILTI

SEQ ID NO: 418 - protein, 240 aa, Artificial Sequence
  Description of Artificial Sequence Synthetic polypeptide
QVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIKGGGC

SEQ ID NO: 419 - protein, 9 aa, Artificial Sequence
  Description of Artificial Sequence Synthetic peptide
VSDGGPNLY

SEQ ID NO: 420 - protein, 9 aa, Artificial Sequence
  Description of Artificial Sequence Synthetic peptide
ATDALMTGY

SEQ ID NO: 421 - protein, 9 aa, Artificial Sequence
  Description of Artificial Sequence Synthetic peptide
IVTDFSVIK

SEQ ID NO: 422 - protein, 9 aa, Artificial Sequence
  Description of Artificial Sequence Synthetic peptide
KSMREEYRK

SEQ ID NO: 423 - protein, 10 aa, Artificial Sequence
  Description of Artificial Sequence Synthetic peptide
SSCSSCPLSK

SEQ ID NO: 424 - protein, 9 aa, Artificial Sequence
  Description of Artificial Sequence Synthetic peptide
ATIGTAMYK

SEQ ID NO: 425 - protein, 10 aa, Artificial Sequence
  Description of Artificial Sequence Synthetic peptide
AVFDRKSDAK

SEQ ID NO: 426 - protein, 9 aa, Artificial Sequence
  Description of Artificial Sequence Synthetic peptide
SIIPSGPLK

SEQ ID NO: 427 - protein, 9 aa, Artificial Sequence
  Description of Artificial Sequence Synthetic peptide
IPSINVHHY

SEQ ID NO: 428 - protein, 11 aa, Artificial Sequence
  Description of Artificial Sequence Synthetic peptide
EPLPQGQLTAY

SEQ ID NO: 429 - protein, 10 aa, Artificial Sequence
  Description of Artificial Sequence Synthetic peptide
VPLDEDFRKY

SEQ ID NO: 430 - protein, 9 aa, Artificial Sequence
  Description of Artificial Sequence Synthetic peptide
RLRAEAQVK

SEQ ID NO: 431 - protein, 9 aa, Artificial Sequence
  Description of Artificial Sequence Synthetic peptide
RLRPGGKKK

SEQ ID NO: 432 - protein, 10 aa, Artificial Sequence
  Description of Artificial Sequence Synthetic peptide
QVPLRPMTYK

SEQ ID NO: 433 - protein, 9 aa, Artificial Sequence
  Description of Artificial Sequence Synthetic peptide
TYGPVFMCL

SEQ ID NO: 434 - protein, 9 aa, Artificial Sequence
  Description of Artificial Sequence Synthetic peptide
RYLKDQQLL

SEQ ID NO: 435 - protein, 9 aa, Artificial Sequence
  Description of Artificial Sequence Synthetic peptide
PYLFWLAAI

SEQ ID NO: 436 - protein, 9 aa, Artificial Sequence
  Description of Artificial Sequence Synthetic peptide
YTFTSYDIN

SEQ ID NO: 437 - protein, 13 aa, Artificial Sequence
  Description of Artificial Sequence Synthetic peptide
GIINPRSGSTKYA

SEQ ID NO: 438 - protein, 16 aa, Artificial Sequence
  Description of Artificial Sequence Synthetic peptide
RSSQSLLHSNGYNYLD

SEQ ID NO: 439 - protein, 7 aa, Artificial Sequence
  Description of Artificial Sequence Synthetic peptide
LGSYRAS

SEQ ID NO: 440 - protein, 4 aa, Homo sapiens
RRVY

SEQ ID NO: 441 - protein, 9 aa, Homo sapiens
FVQENYLEY

SEQ ID NO: 442 - protein, 9 aa, Homo sapiens
LVHFLLLKY

SEQ ID NO: 443 - protein, 10 aa, Homo sapiens
MEVDPIGHLY

SEQ ID NO: 444 - protein, 9 aa, Homo sapiens
EVDPASNTY

SEQ ID NO: 445 - protein, 9 aa, Homo sapiens
KVLEYVIKV

SEQ ID NO: 446 - protein, 9 aa, Homo sapiens
LVQENYLEY

SEQ ID NO: 447 - protein, 9 aa, Homo sapiens
AFDDIATYF

SEQ ID NO: 448 - protein, 9 aa, Homo sapiens
WVQENYLEY

SEQ ID NO: 449 - protein, 9 aa, Homo sapiens
SLFRAVITK

SEQ ID NO: 450 - protein, 9 aa, Homo sapiens
ALAETSYVK

SEQ ID NO: 451 - protein, 9 aa, Homo sapiens
LYATVIHDI

SEQ ID NO: 452 - protein, 10 aa, Homo sapiens
QLLDGFMITL

SEQ ID NO: 453 - protein, 9 aa, Homo sapiens
STLPTTINY

SEQ ID NO: 454 - protein, 10 aa, Homo sapiens
YPAPLESLDY

SEQ ID NO: 455 - protein, 9 aa, Homo sapiens
ATLENLLSH

SEQ ID NO: 456 - protein, 8 aa, Homo sapiens
DALLAQKV

SEQ ID NO: 457 - protein, 10 aa, Homo sapiens
SESDLKHLSW

SEQ ID NO: 458 - protein, 9 aa, Homo sapiens
TLDEYLTYL

SEQ ID NO: 459 - protein, 9 aa, Homo sapiens
RQKRILVNL

SEQ ID NO: 460 - protein, 9 aa, Artificial Sequence
  Description of Artificial Sequence Synthetic peptide
YTFTSHDIN

SEQ ID NO: 461 - protein, 13 aa, Artificial Sequence
  Description of Artificial Sequence Synthetic peptide
GWMNPNSGDTGYA

SEQ ID NO: 462 - protein, 7 aa, Artificial Sequence
  Description of Artificial Sequence Synthetic peptide
LGSSRAS

SEQ ID NO: 463 - protein, 9 aa, Artificial Sequence
  Description of Artificial Sequence Synthetic peptide
FSFSSYWMS

SEQ ID NO: 464 - protein, 13 aa, Artificial Sequence
  Description of Artificial Sequence Synthetic peptide
SYISGDSGYTNYA

SEQ ID NO: 465 - protein, 11 aa, Artificial Sequence
  Description of Artificial Sequence Synthetic peptide
QASQDISNYLN

SEQ ID NO: 466 - protein, 7 aa, Artificial Sequence
  Description of Artificial Sequence Synthetic peptide
AASSLQS

SEQ ID NO: 467 - protein, 9 aa, Artificial Sequence
  Description of Artificial Sequence Synthetic peptide
FTFSNSDMN

SEQ ID NO: 468 - protein, 13 aa, Artificial Sequence
  Description of Artificial Sequence Synthetic peptide
AYISSGSSTIYYA

SEQ ID NO: 469 - protein, 11 aa, Artificial Sequence
  Description of Artificial Sequence Synthetic peptide
RASQSISSWLA

SEQ ID NO: 470 - protein, 7 aa, Artificial Sequence
  Description of Artificial Sequence Synthetic peptide
SASTLQS

SEQ ID NO: 471 - protein, 13 aa, Artificial Sequence
  Description of Artificial Sequence Synthetic peptide
ASISSSGGYINYA

SEQ ID NO: 472 - protein, 9 aa, Artificial Sequence
  Description of Artificial Sequence Synthetic peptide
GTFSNFGVS

SEQ ID NO: 473 - protein, 13 aa, Artificial Sequence
  Description of Artificial Sequence Synthetic peptide
GGIIPILGTANYA

SEQ ID NO: 474 - protein, 7 aa, Artificial Sequence
  Description of Artificial Sequence Synthetic peptide
AASTLQS

SEQ ID NO: 475 - protein, 9 aa, Artificial Sequence
  Description of Artificial Sequence Synthetic peptide
YTFTSYNMH

SEQ ID NO: 476 - protein, 13 aa, Artificial Sequence
  Description of Artificial Sequence Synthetic peptide
GWINPNSGGTNYA

SEQ ID NO: 477 - protein, 11 aa, Artificial Sequence
  Description of Artificial Sequence Synthetic peptide
RASQGISNYLN

SEQ ID NO: 478 - protein, 7 aa, Artificial Sequence
  Description of Artificial Sequence Synthetic peptide
YASSLQS

SEQ ID NO: 479 - protein, 9 aa, Artificial Sequence
  Description of Artificial Sequence Synthetic peptide
GTFSGYLVS

SEQ ID NO: 480 - protein, 13 aa, Artificial Sequence
  Description of Artificial Sequence Synthetic peptide
GWINPNSGGTNTA

SEQ ID NO: 481 - protein, 11 aa, Artificial Sequence
  Description of Artificial Sequence Synthetic peptide
RASQSISSYLN

SEQ ID NO: 482 - protein, 7 aa, Artificial Sequence
  Description of Artificial Sequence Synthetic peptide
GASSLQS

SEQ ID NO: 483 - protein, 9 aa, Artificial Sequence
  Description of Artificial Sequence Synthetic peptide
YIFRNYPMH

SEQ ID NO: 484 - protein, 13 aa, Artificial Sequence
  Description of Artificial Sequence Synthetic peptide
GWINPDSGGTKYA

SEQ ID NO: 485 - protein, 11 aa, Artificial Sequence
  Description of Artificial Sequence Synthetic peptide
RASQGISNYLA

SEQ ID NO: 486 - protein, 9 aa, Artificial Sequence
  Description of Artificial Sequence Synthetic peptide
YTFTGYYMH

SEQ ID NO: 487 - protein, 13 aa, Artificial Sequence
  Description of Artificial Sequence Synthetic peptide
GWMNPNIGNTGYA

SEQ ID NO: 488 - protein, 17 aa, Artificial Sequence
  Description of Artificial Sequence Synthetic peptide
KTSQSVLYRPNNENYLA

SEQ ID NO: 489 - protein, 7 aa, Artificial Sequence
  Description of Artificial Sequence Synthetic peptide
QASIREP

SEQ ID NO: 490 - protein, 9 aa, Artificial Sequence
  Description of Artificial Sequence Synthetic peptide
GTFSSYGIS

SEQ ID NO: 491 - protein, 13 aa, Artificial Sequence
  Description of Artificial Sequence Synthetic peptide
GWINPNSGVTKYA

SEQ ID NO: 492 - protein, 11 aa, Artificial Sequence
  Description of Artificial Sequence Synthetic peptide
RASQSISRFLN

SEQ ID NO: 493 - protein, 7 aa, Artificial Sequence
  Description of Artificial Sequence Synthetic peptide
GASRPQS

SEQ ID NO: 494 - protein, 13 aa, Artificial Sequence
  Description of Artificial Sequence Synthetic peptide
GWINPNSGDTKYS

SEQ ID NO: 495 - protein, 7 aa, Artificial Sequence
  Description of Artificial Sequence Synthetic peptide
LGSHRAS

SEQ ID NO: 496 - protein, 9 aa, Artificial Sequence
  Description of Artificial Sequence Synthetic peptide
FTFSDYYMS

SEQ ID NO: 497 - protein, 13 aa, Artificial Sequence
  Description of Artificial Sequence Synthetic peptide
SYISSSSSYTNYA

SEQ ID NO: 498 - protein, 11 aa, Artificial Sequence
  Description of Artificial Sequence Synthetic peptide
RASQSVSSNLA

SEQ ID NO: 499 - protein, 7 aa, Artificial Sequence
  Description of Artificial Sequence Synthetic peptide
AASARAS

SEQ ID NO: 500 - protein, 9 aa, Artificial Sequence
  Description of Artificial Sequence Synthetic peptide
GTFSSYAIS

SEQ ID NO: 501 - protein, 13 aa, Artificial Sequence
  Description of Artificial Sequence Synthetic peptide
GWMNPDSGSTGYA

SEQ ID NO: 502 - protein, 7 aa, Artificial Sequence
  Description of Artificial Sequence Synthetic peptide
GASRLQS

SEQ ID NO: 503 - protein, 9 aa, Artificial Sequence
  Description of Artificial Sequence Synthetic peptide
FTFTSYSMH

SEQ ID NO: 504 - protein, 13 aa, Artificial Sequence
  Description of Artificial Sequence Synthetic peptide
SSITSFTNTMYYA

SEQ ID NO: 505 - protein, 7 aa, Artificial Sequence
  Description of Artificial Sequence Synthetic peptide
LGSNRAS

SEQ ID NO: 506 - protein, 9 aa, Artificial Sequence
  Description of Artificial Sequence Synthetic peptide
YTFTNYYMH

SEQ ID NO: 507 - protein, 13 aa, Artificial Sequence
  Description of Artificial Sequence Synthetic peptide
GIINPSGGSTSYA

SEQ ID NO: 508 - protein, 11 aa, Artificial Sequence
  Description of Artificial Sequence Synthetic peptide
QASEDISNHLN

SEQ ID NO: 509 - protein, 7 aa, Artificial Sequence
  Description of Artificial Sequence Synthetic peptide
DALSLQS

SEQ ID NO: 510 - protein, 9 aa, Artificial Sequence
  Description of Artificial Sequence Synthetic peptide
YTFTSYYMH

SEQ ID NO: 511 - protein, 13 aa, Artificial Sequence
  Description of Artificial Sequence Synthetic peptide
GWMNPNSGNTGYA

SEQ ID NO: 512 - protein, 13 aa, Artificial Sequence
  Description of Artificial Sequence Synthetic peptide
GGIIPVMGTGNYA

SEQ ID NO: 513 - protein, 9 aa, Artificial Sequence
  Description of Artificial Sequence Synthetic peptide
GTFSRSAIT

SEQ ID NO: 514 - protein, 13 aa, Artificial Sequence
  Description of Artificial Sequence Synthetic peptide
GWINPNSGATNYA

SEQ ID NO: 515 - protein, 11 aa, Artificial Sequence
  Description of Artificial Sequence Synthetic peptide
RASQSITSYLN

SEQ ID NO: 516 - protein, 7 aa, Artificial Sequence
  Description of Artificial Sequence Synthetic peptide
DASNLET

SEQ ID NO: 517 - protein, 9 aa, Artificial Sequence
  Description of Artificial Sequence Synthetic peptide
YPFIGQYLH

SEQ ID NO: 518 - protein, 13 aa, Artificial Sequence
  Description of Artificial Sequence Synthetic peptide
GIINPSGDSATYA

SEQ ID NO: 519 - protein, 11 aa, Artificial Sequence
  Description of Artificial Sequence Synthetic peptide
WASQGISSYLA

SEQ ID NO: 520 - protein, 13 aa, Artificial Sequence
  Description of Artificial Sequence Synthetic peptide
GWMNPIGGGTGYA

SEQ ID NO: 521 - protein, 11 aa, Artificial Sequence
  Description of Artificial Sequence Synthetic peptide
RASQAISNSLA

SEQ ID NO: 522 - protein, 13 aa, Artificial Sequence
  Description of Artificial Sequence Synthetic peptide
SGINWNGGSTGYA

SEQ ID NO: 523 - protein, 7 aa, Artificial Sequence
  Description of Artificial Sequence Synthetic peptide
KASSLES

SEQ ID NO: 524 - protein, 9 aa, Artificial Sequence
  Description of Artificial Sequence Synthetic peptide
GTLSSYPIN

SEQ ID NO: 525 - protein, 13 aa, Artificial Sequence
  Description of Artificial Sequence Synthetic peptide
GWISTYSGHADYA

SEQ ID NO: 526 - protein, 9 aa, Artificial Sequence
  Description of Artificial Sequence Synthetic peptide
FTFSSYWMS

SEQ ID NO: 527 - protein, 13 aa, Artificial Sequence
  Description of Artificial Sequence Synthetic peptide
SSISGRGDNTYYA

SEQ ID NO: 528 - protein, 9 aa, Artificial Sequence
  Description of Artificial Sequence Synthetic peptide
YTFGNYFMH

SEQ ID NO: 529 - protein, 13 aa, Artificial Sequence
  Description of Artificial Sequence Synthetic peptide
GMVNPSGGSETFA

SEQ ID NO: 530 - protein, 11 aa, Artificial Sequence
  Description of Artificial Sequence Synthetic peptide
RASQGINSYLA

SEQ ID NO: 531 - protein, 9 aa, Artificial Sequence
  Description of Artificial Sequence Synthetic peptide
FDFSIYSMN

SEQ ID NO: 532 - protein, 13 aa, Artificial Sequence
  Description of Artificial Sequence Synthetic peptide
SAISGSGGSTYYA

SEQ ID NO: 533 - protein, 11 aa, Artificial Sequence
  Description of Artificial Sequence Synthetic peptide
RASQSISRWLA

SEQ ID NO: 534 - protein, 9 aa, Artificial Sequence
  Description of Artificial Sequence Synthetic peptide
YTLTTYYMH

SEQ ID NO: 535 - protein, 11 aa, Artificial Sequence
  Description of Artificial Sequence Synthetic peptide
RASQGISNSLA

SEQ ID NO: 536 - protein, 13 aa, Artificial Sequence
  Description of Artificial Sequence Synthetic peptide
GWINPYSGGTNYA

SEQ ID NO: 537 - protein, 11 aa, Artificial Sequence
  Description of Artificial Sequence Synthetic peptide
RASQDVSTWLA

SEQ ID NO: 538 - protein, 9 aa, Artificial Sequence
  Description of Artificial Sequence Synthetic peptide
GTFSSYGVS

SEQ ID NO: 539 - protein, 13 aa, Artificial Sequence
  Description of Artificial Sequence Synthetic peptide
GWISPYSGNTDYA

SEQ ID NO: 540 - protein, 9 aa, Artificial Sequence
  Description of Artificial Sequence Synthetic peptide
YTFSNMYLH

SEQ ID NO: 541 - protein, 13 aa, Artificial Sequence
  Description of Artificial Sequence Synthetic peptide
GWINPNTGDTNYA

SEQ ID NO: 542 - protein, 11 aa, Artificial Sequence
  Description of Artificial Sequence Synthetic peptide
RASQGISNWLA

SEQ ID NO: 543 - protein, 9 aa, Artificial Sequence
  Description of Artificial Sequence Synthetic peptide
YTFTGYYIH

SEQ ID NO: 544 - protein, 13 aa, Artificial Sequence
  Description of Artificial Sequence Synthetic peptide
GVINPSGGSTTYA

SEQ ID NO: 545 - protein, 11 aa, Artificial Sequence
  Description of Artificial Sequence Synthetic peptide
RASQSVGNSLA

SEQ ID NO: 546 - protein, 7 aa, Artificial Sequence
  Description of Artificial Sequence Synthetic peptide
GASTRAT

SEQ ID NO: 547 - protein, 9 aa, Artificial Sequence
  Description of Artificial Sequence Synthetic peptide
YTFTSNYMH

SEQ ID NO: 548 - protein, 11 aa, Artificial Sequence
  Description of Artificial Sequence Synthetic peptide
RASQSISGYLN

SEQ ID NO: 549 - protein, 9 aa, Artificial Sequence
  Description of Artificial Sequence Synthetic peptide
GTFSSHAIS

SEQ ID NO: 550 - protein, 12 aa, Artificial Sequence
  Description of Artificial Sequence Synthetic peptide
GVIIPSGGTSYT

SEQ ID NO: 551 - protein, 11 aa, Artificial Sequence
  Description of Artificial Sequence Synthetic peptide
RASQNIYTYLN

SEQ ID NO: 552 - protein, 9 aa, Artificial Sequence
  Description of Artificial Sequence Synthetic peptide
YTFTSYAMN

SEQ ID NO: 553 - protein, 13 aa, Artificial Sequence
  Description of Artificial Sequence Synthetic peptide
SGISARSGRTYYA

SEQ ID NO: 554 - protein, 7 aa, Artificial Sequence
  Description of Artificial Sequence Synthetic peptide
AASSLQG

SEQ ID NO: 555 - protein, 13 aa, Artificial Sequence
  Description of Artificial Sequence Synthetic peptide
GIIHPGGGTTSYA

SEQ ID NO: 556 - protein, 7 aa, Artificial Sequence
  Description of Artificial Sequence Synthetic peptide
DASRLQS

SEQ ID NO: 557 - protein, 9 aa, Artificial Sequence
  Description of Artificial Sequence Synthetic peptide
YIFTGYYMH

SEQ ID NO: 558 - protein, 13 aa, Artificial Sequence
  Description of Artificial Sequence Synthetic peptide
GMIGPSDGSTSYA

SEQ ID NO: 559 - protein, 9 aa, Artificial Sequence
  Description of Artificial Sequence Synthetic peptide
YTFIGYYMH

SEQ ID NO: 560 - protein, 11 aa, Artificial Sequence
  Description of Artificial Sequence Synthetic peptide
RASQSISNYLN

SEQ ID NO: 561 - protein, 9 aa, Artificial Sequence
  Description of Artificial Sequence Synthetic peptide
VTFSTSAIS

SEQ ID NO: 562 - protein, 13 aa, Artificial Sequence
  Description of Artificial Sequence Synthetic peptide
GWISPYNGNTDYA

SEQ ID NO: 563 - protein, 7 aa, Artificial Sequence
  Description of Artificial Sequence Synthetic peptide
SASNLQS

SEQ ID NO: 564 - protein, 9 aa, Artificial Sequence
  Description of Artificial Sequence Synthetic peptide
GTFSNSIIN

SEQ ID NO: 565 - protein, 13 aa, Artificial Sequence
  Description of Artificial Sequence Synthetic peptide
GWMNPNSGNTNYA

SEQ ID NO: 566 - protein, 11 aa, Artificial Sequence
  Description of Artificial Sequence Synthetic peptide
RASQNISSYLN

SEQ ID NO: 567 - protein, 9 aa, Artificial Sequence
  Description of Artificial Sequence Synthetic peptide
GTFSTHDIN

SEQ ID NO: 568 - protein, 13 aa, Artificial Sequence
  Description of Artificial Sequence Synthetic peptide
GVINPSGGSAIYA

SEQ ID NO: 569 - protein, 11 aa, Artificial Sequence
  Description of Artificial Sequence Synthetic peptide
RASQDISRYLA

SEQ ID NO: 570 - protein, 9 aa, Artificial Sequence
  Description of Artificial Sequence Synthetic peptide
NTFIGYYVH

SEQ ID NO: 571 - protein, 13 aa, Artificial Sequence
  Description of Artificial Sequence Synthetic peptide
GIINPNGGSISYA

SEQ ID NO: 572 - protein, 7 aa, Artificial Sequence
  Description of Artificial Sequence Synthetic peptide
AASNLQS

SEQ ID NO: 573 - protein, 9 aa, Artificial Sequence
  Description of Artificial Sequence Synthetic peptide
YTLSYYYMH

SEQ ID NO: 574 - protein, 7 aa, Artificial Sequence
  Description of Artificial Sequence Synthetic peptide
AASTLQN

SEQ ID NO: 575 - protein, 13 aa, Artificial Sequence
  Description of Artificial Sequence Synthetic peptide
GIIGPSDGSTTYA

SEQ ID NO: 576 - protein, 11 aa, Artificial Sequence
  Description of Artificial Sequence Synthetic peptide
RASQRISSYLN

SEQ ID NO: 577 - protein, 9 aa, Artificial Sequence
  Description of Artificial Sequence Synthetic peptide
YTFTGYYVH

SEQ ID NO: 578 - protein, 13 aa, Artificial Sequence
  Description of Artificial Sequence Synthetic peptide
GIIAPSDGSTNYA

SEQ ID NO: 579 - protein, 11 aa, Artificial Sequence
  Description of Artificial Sequence Synthetic peptide
RASQSISSYLA

SEQ ID NO: 580 - protein, 7 aa, Artificial Sequence
  Description of Artificial Sequence Synthetic peptide
DASKLET

SEQ ID NO: 581 - protein, 9 aa, Artificial Sequence
  Description of Artificial Sequence Synthetic peptide
YTFTGYYLH

SEQ ID NO: 582 - protein, 11 aa, Artificial Sequence
  Description of Artificial Sequence Synthetic peptide
RASQGISSWLA

SEQ ID NO: 583 - protein, 13 aa, Artificial Sequence
  Description of Artificial Sequence Synthetic peptide
GRISPSDGSTTYA

SEQ ID NO: 584 - protein, 11 aa, Artificial Sequence
  Description of Artificial Sequence Synthetic peptide
RASQGISTYLA

SEQ ID NO: 585 - protein, 7 aa, Artificial Sequence
  Description of Artificial Sequence Synthetic peptide
DASSLQS

SEQ ID NO: 586 - protein, 9 aa, Artificial Sequence
  Description of Artificial Sequence Synthetic peptide
GTFNNFAIS

SEQ ID NO: 587 - protein, 13 aa, Artificial Sequence
  Description of Artificial Sequence Synthetic peptide
GGIIPIFDATNYA

SEQ ID NO: 588 - protein, 9 aa, Artificial Sequence
  Description of Artificial Sequence Synthetic peptide
YNFTGYYMH

SEQ ID NO: 589 - protein, 9 aa, Artificial Sequence
  Description of Artificial Sequence Synthetic peptide
GTFSSATIS

SEQ ID NO: 590 - protein, 13 aa, Artificial Sequence
  Description of Artificial Sequence Synthetic peptide
GWIYPNSGGTVYA

SEQ ID NO: 591 - protein, 11 aa, Artificial Sequence
  Description of Artificial Sequence Synthetic peptide
RASQSISTWLA

SEQ ID NO: 592 - protein, 7 aa, Artificial Sequence
  Description of Artificial Sequence Synthetic peptide
AASSLRS

SEQ ID NO: 593 - protein, 13 aa, Artificial Sequence
  Description of Artificial Sequence Synthetic peptide
GWINPNSGGTISA

SEQ ID NO: 594 - protein, 7 aa, Artificial Sequence
  Description of Artificial Sequence Synthetic peptide
AASTVQS

SEQ ID NO: 595 - protein, 9 aa, Artificial Sequence
  Description of Artificial Sequence Synthetic peptide
YTFTTYDLA

SEQ ID NO: 596 - protein, 11 aa, Artificial Sequence
  Description of Artificial Sequence Synthetic peptide
RASQDISRWLA

SEQ ID NO: 597 - protein, 7 aa, Artificial Sequence
  Description of Artificial Sequence Synthetic peptide
AASRLQA

SEQ ID NO: 598 - protein, 9 aa, Artificial Sequence
  Description of Artificial Sequence Synthetic peptide
YSFDSYVVN

SEQ ID NO: 599 - protein, 11 aa, Artificial Sequence
  Description of Artificial Sequence Synthetic peptide
RASQTISSWLA

SEQ ID NO: 600 - protein, 9 aa, Artificial Sequence
  Description of Artificial Sequence Synthetic peptide
YTFTSYGIS

SEQ ID NO: 601 - protein, 13 aa, Artificial Sequence
  Description of Artificial Sequence Synthetic peptide
GWMNPNSGGTNYA

SEQ ID NO: 602 - protein, 11 aa, Artificial Sequence
  Description of Artificial Sequence Synthetic peptide
RASQSISNWLA

SEQ ID NO: 603 - protein, 9 aa, Artificial Sequence
  Description of Artificial Sequence Synthetic peptide
YTFTRYTIN

SEQ ID NO: 604 - protein, 11 aa, Artificial Sequence
  Description of Artificial Sequence Synthetic peptide
RASQSVGNWLA

SEQ ID NO: 605 - protein, 7 aa, Artificial Sequence
  Description of Artificial Sequence Synthetic peptide
GASSLQT

SEQ ID NO: 606 - protein, 9 aa, Artificial Sequence
  Description of Artificial Sequence Synthetic peptide
YTFTSYGIT

SEQ ID NO: 607 - protein, 11 aa, Artificial Sequence
  Description of Artificial Sequence Synthetic peptide
RASQNIGNWLA

SEQ ID NO: 608 - protein, 7 aa, Artificial Sequence
  Description of Artificial Sequence Synthetic peptide
AASTLQT

SEQ ID NO: 609 - protein, 9 aa, Artificial Sequence
  Description of Artificial Sequence Synthetic peptide
GTFSNYILS

SEQ ID NO: 610 - protein, 13 aa, Artificial Sequence
  Description of Artificial Sequence Synthetic peptide
GWINPDSGGTNYA

SEQ ID NO: 611 - protein, 9 aa, Artificial Sequence
  Description of Artificial Sequence Synthetic peptide
YSFTRYNMH

SEQ ID NO: 612 - protein, 13 aa, Artificial Sequence
  Description of Artificial Sequence Synthetic peptide
GWINPNNGGTNYA

SEQ ID NO: 613 - protein, 11 aa, Artificial Sequence
  Description of Artificial Sequence Synthetic peptide
RASQSISKWLA

SEQ ID NO: 614 - protein, 9 aa, Artificial Sequence
  Description of Artificial Sequence Synthetic peptide
GTFNNYAFS

SEQ ID NO: 615 - protein, 9 aa, Artificial Sequence
  Description of Artificial Sequence Synthetic peptide
YTFSSYNMH

SEQ ID NO: 616 - protein, 13 aa, Artificial Sequence
  Description of Artificial Sequence Synthetic peptide
GWINGNTGGTNYA

SEQ ID NO: 617 - protein, 11 aa, Artificial Sequence
  Description of Artificial Sequence Synthetic peptide
RASQTISNYLN

SEQ ID NO: 618 - protein, 9 aa, Artificial Sequence
  Description of Artificial Sequence Synthetic peptide
GTFSSYAFS

SEQ ID NO: 619 - protein, 13 aa, Artificial Sequence
  Description of Artificial Sequence Synthetic peptide
GWINPDTGYTRYA

SEQ ID NO: 620 - protein, 11 aa, Artificial Sequence
  Description of Artificial Sequence Synthetic peptide
RASRDIGRAVG

SEQ ID NO: 621 - protein, 13 aa, Artificial Sequence
  Description of Artificial Sequence Synthetic peptide
GWISAYNGYTNYA

SEQ ID NO: 622 - protein, 9 aa, Artificial Sequence
  Description of Artificial Sequence Synthetic peptide
YTFTDYNMH

SEQ ID NO: 623 - protein, 11 aa, Artificial Sequence
  Description of Artificial Sequence Synthetic peptide
RASQSIGRWLA

SEQ ID NO: 624 - protein, 13 aa, Artificial Sequence
  Description of Artificial Sequence Synthetic peptide
GWINPNSGGANYA

SEQ ID NO: 625 - protein, 9 aa, Artificial Sequence
  Description of Artificial Sequence Synthetic peptide
YTFTSYLMH

SEQ ID NO: 626 - protein, 13 aa, Artificial Sequence
  Description of Artificial Sequence Synthetic peptide
GWISPNSGGTNYA

SEQ ID NO: 627 - protein, 9 aa, Artificial Sequence
  Description of Artificial Sequence Synthetic peptide
YTFSDYYVH

SEQ ID NO: 628 - protein, 9 aa, Artificial Sequence
  Description of Artificial Sequence Synthetic peptide
YTFTTYYMH

SEQ ID NO: 629 - protein, 11 aa, Artificial Sequence
  Description of Artificial Sequence Synthetic peptide
RASQSVSNWLA

SEQ ID NO: 630 - protein, 9 aa, Artificial Sequence
  Description of Artificial Sequence Synthetic peptide
GTFSNYAIN

SEQ ID NO: 631 - protein, 13 aa, Artificial Sequence
  Description of Artificial Sequence Synthetic peptide
GWISPYSGGTNYA

SEQ ID NO: 632 - protein, 9 aa, Artificial Sequence
  Description of Artificial Sequence Synthetic peptide
YTFTDYYMH

SEQ ID NO: 633 - protein, 13 aa, Artificial Sequence
  Description of Artificial Sequence Synthetic peptide
GWIYPNTGGTNYA

SEQ ID NO: 634 - protein, 11 aa, Artificial Sequence
  Description of Artificial Sequence Synthetic peptide
QASQDIGSWLA

SEQ ID NO: 635 - protein, 7 aa, Artificial Sequence
  Description of Artificial Sequence Synthetic peptide
ATSSLQS

SEQ ID NO: 636 - protein, 13 aa, Artificial Sequence
  Description of Artificial Sequence Synthetic peptide
GWMNPNSGGTKYA

SEQ ID NO: 637 - protein, 11 aa, Artificial Sequence
  Description of Artificial Sequence Synthetic peptide
RASQGISRWLA

SEQ ID NO: 638 - protein, 7 aa, Artificial Sequence
  Description of Artificial Sequence Synthetic peptide
AASTLQP

SEQ ID NO: 639 - protein, 9 aa, Artificial Sequence
  Description of Artificial Sequence Synthetic peptide
YTLTSHLIH

SEQ ID NO: 640 - protein, 7 aa, Artificial Sequence
  Description of Artificial Sequence Synthetic peptide
AASRLES

SEQ ID NO: 641 - protein, 9 aa, Artificial Sequence
  Description of Artificial Sequence Synthetic peptide
YSFTDYIVH

SEQ ID NO: 642 - protein, 13 aa, Artificial Sequence
  Description of Artificial Sequence Synthetic peptide
GWINPYSGGTKYA

SEQ ID NO: 643 - protein, 7 aa, Artificial Sequence
  Description of Artificial Sequence Synthetic peptide
GVSSLQS

SEQ ID NO: 644 - protein, 9 aa, Artificial Sequence
  Description of Artificial Sequence Synthetic peptide
YTFSNFLIN

SEQ ID NO: 645 - protein, 11 aa, Artificial Sequence
  Description of Artificial Sequence Synthetic peptide
RASQSISSWVA

SEQ ID NO: 646 - protein, 7 aa, Artificial Sequence
  Description of Artificial Sequence Synthetic peptide
GASNLES

SEQ ID NO: 647 - protein, 9 aa, Artificial Sequence
  Description of Artificial Sequence Synthetic peptide
YTFTDYQMF

SEQ ID NO: 648 - protein, 9 aa, Artificial Sequence
  Description of Artificial Sequence Synthetic peptide
YTFTNYHMH

SEQ ID NO: 649 - protein, 13 aa, Artificial Sequence
  Description of Artificial Sequence Synthetic peptide
GWIHPDSGGTSYA

SEQ ID NO: 650 - protein, 11 aa, Artificial Sequence
  Description of Artificial Sequence Synthetic peptide
RASQSISHYLN

SEQ ID NO: 651 - protein, 9 aa, Artificial Sequence
  Description of Artificial Sequence Synthetic peptide
YTFTSYTVN

SEQ ID NO: 652 - protein, 13 aa, Artificial Sequence
  Description of Artificial Sequence Synthetic peptide
GWINPNSGGTKYA

SEQ ID NO: 653 - protein, 13 aa, Artificial Sequence
  Description of Artificial Sequence Synthetic peptide
GMINPRDDTTDYA

SEQ ID NO: 654 - protein, 13 aa, Artificial Sequence
  Description of Artificial Sequence Synthetic peptide
GMINPSGGGTSYA

SEQ ID NO: 655 - protein, 9 aa, Artificial Sequence
  Description of Artificial Sequence Synthetic peptide
YTFTSQYMH

SEQ ID NO: 656 - protein, 13 aa, Artificial Sequence
  Description of Artificial Sequence Synthetic peptide
GRIIPLLGIVNYA

SEQ ID NO: 657 - protein, 11 aa, Artificial Sequence
  Description of Artificial Sequence Synthetic peptide
RASQSVSRNLA

SEQ ID NO: 658 - protein, 9 aa, Artificial Sequence
  Description of Artificial Sequence Synthetic peptide
GTFSNYGIS

SEQ ID NO: 659 - protein, 12 aa, Artificial Sequence
  Description of Artificial Sequence Synthetic peptide
GIINPGGSTSYA

SEQ ID NO: 660 - protein, 12 aa, Artificial Sequence
  Description of Artificial Sequence Synthetic peptide
RASQSVSSSNLA

SEQ ID NO: 661 - protein, 9 aa, Artificial Sequence
  Description of Artificial Sequence Synthetic peptide
FTFSSYAMH

SEQ ID NO: 662 - protein, 13 aa, Artificial Sequence
  Description of Artificial Sequence Synthetic peptide
SGISGSGGSTYYA

SEQ ID NO: 663 - protein, 11 aa, Artificial Sequence
  Description of Artificial Sequence Synthetic peptide
RASQDIRNDLG

SEQ ID NO: 664 - protein, 9 aa, Artificial Sequence
  Description of Artificial Sequence Synthetic peptide
YTFSNYYIH

SEQ ID NO: 665 - protein, 13 aa, Artificial Sequence
  Description of Artificial Sequence Synthetic peptide
GWLNPNSGNTGYA

SEQ ID NO: 666 - protein, 17 aa, Artificial Sequence
  Description of Artificial Sequence Synthetic peptide
KSSQSVFYSSNNKNQLA

SEQ ID NO: 667 - protein, 7 aa, Artificial Sequence
  Description of Artificial Sequence Synthetic peptide
WASTRES

SEQ ID NO: 668 - protein, 9 aa, Artificial Sequence
  Description of Artificial Sequence Synthetic peptide
YTFTTDGIS

SEQ ID NO: 669 - protein, 13 aa, Artificial Sequence
  Description of Artificial Sequence Synthetic peptide
GRIYPHSGYTEYA

SEQ ID NO: 670 - protein, 11 aa, Artificial Sequence
  Description of Artificial Sequence Synthetic peptide
QASQDIFKYLN

SEQ ID NO: 671 - protein, 13 aa, Artificial Sequence
  Description of Artificial Sequence Synthetic peptide
GWISPNNGDTNYA

SEQ ID NO: 672 - protein, 13 aa, Artificial Sequence
  Description of Artificial Sequence Synthetic peptide
GRIIPMLNIANYA

SEQ ID NO: 673 - protein, 16 aa, Artificial Sequence
  Description of Artificial Sequence Synthetic peptide
SSSQSLLHSNGYNYLD

SEQ ID NO: 674 - protein, 9 aa, Artificial Sequence
  Description of Artificial Sequence Synthetic peptide
FTFSSSWMH

SEQ ID NO: 675 - protein, 13 aa, Artificial Sequence
  Description of Artificial Sequence Synthetic peptide
SFISTSSGYIYYA

SEQ ID NO: 676 - protein, 7 aa, Artificial Sequence
  Description of Artificial Sequence Synthetic peptide
SASNLRS

SEQ ID NO: 677 - protein, 9 aa, Artificial Sequence
  Description of Artificial Sequence Synthetic peptide
DTFNTYALS

SEQ ID NO: 678 - protein, 13 aa, Artificial Sequence
  Description of Artificial Sequence Synthetic peptide
GWMNPNSGNAGYA

SEQ ID NO: 679 - DNA, 1660 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactccgacatccagatgacccagagcccaagcagcctgagcgccagcgtgggcgacagggtgaccatcacctgccaggccagccaggacatctcaaactacctgaactggtatcaacagaagcccggcaaggcccccaagttgcttatctacgccgcgagcagcttgcaaagcggcgtgcccagcaggttcagcggtagtggcagtggcaccgactttaccctgaccataagcagtctgcagcccgaggacttcgccacctactactgccagcagtactacagctaccccttcaccttcggacccggaaccaaggtggacatcaaaggaggcggaggatctggtggtggtggatctggcggcggaggaagtggtggcggaggctctcaggtgcagctggtgcagagcggagccgaggtgaagaaacctggggcaagcgtgaaagtgagctgcaaggccagcggctacaccttcaccaactactacatgcactgggtgaggcaggcacctggtcaaggcctggagtggatgggcatgatcaaccccagcggaggagggaccagctacgcccagaagttccaaggccgcgtgaccatgactagggatacctccaccagcaccgtgtatatggagctgagcagcctgaggagcgaggacaccgccgtgtattactgcgccaggggcaatccctgggaactgagactggactactgggggcagggcaccctcgtgacagtgtcttctggcggagggggatcccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcctcagtggagggcggctccggcggctccggtggctctggcggaagcggcggcgtggaccaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaacggcggagggggctctcatcatcaccatcaccatcatcac

SEQ ID NO: 680 - protein, 526 aa, Artificial Sequence
  Description of Artificial Sequence Synthetic polypeptide
MGWSCIILFLVATATGVHSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIKGGGGSGGGGSGGGGSGGGGSQVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSVEGGSGGSGGSGGSGGVDQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSHHHHHHHH

SEQ ID NO: 681 - protein, 6 aa, Artificial Sequence
  Description of Artificial Sequence Synthetic 6xHis tag
HHHHHH

SEQ ID NO: 682 - DNA, 1666 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactccgacatccagatgacccagagcccctccagtcttagcgccagcgtgggcgacagggtgaccatcacctgccaggccagccaggacatcagcaactacctgaattggtatcagcagaagcccggcaaggcccccaagctcctgatctacgccgcgagctcactgcagagcggcgtgcccagcaggttcagtggcagcggaagcgggaccgacttcacgctgaccattagcagcctccagcctgaggacttcgccacctactactgccagcaagccatcagcttccccctgactttcggccagagcaccaaggtggagatcaagggaggcggaggatctggtggtggtggatctggcggcggaggaagtggtggcggaggctctgaggtgcagcttcttgagtctggcggtggtctggtgaagcccggtggcagcctgcgcctcagctgtgccgcaagcggcttcagtttcagcagctactggatgagctgggtgaggcaggcacccggcaagggccttgagtggatcagctacatcagcggcgactctggctacaccaactacgccgacagcgttaagggcaggttcaccatcagcagggacgatagcaagaacaccctgtacctgcagatgaatagcctgaagaccgaagacaccgccgtgtactactgcgccagccacgattacggagactatggagagtacttccagcactggggccagggcaccctggtgacggtgagctctggcggagggggatcccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcctcagtggagggcggctccggcggctccggtggctctggcggaagcggcggcgtggaccaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaacggcggagggggctctcatcatcaccatcaccatcatcac

SEQ ID NO: 683 - protein, 528 aa, Artificial Sequence
  Description of Artificial Sequence Synthetic polypeptide
MGWSCIILFLVATATGVHSDIQMTQSPSSLSASVGDRVTITCQASQDISNYLNWYQQKPGKAPKLLIYAASSLQSGVPSRFSGSGSGTDFTLTISSLQPEDFATYYCQQAISFPLTFGQSTKVEIKGGGGSGGGGSGGGGSGGGGSEVQLLESGGGLVKPGGSLRLSCAASGFSFSSYWMSWVRQAPGKGLEWISYISGDSGYTNYADSVKGRFTISRDDSKNTLYLQMNSLKTEDTAVYYCASHDYGDYGEYFQHWGQGTLVTVSSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSVEGGSGGSGGSGGSGGVDQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSHHHHHHHH

SEQ ID NO: 684 - DNA, 1588 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactcccaggtgcagctggtgcagagcggagccgaggtgaagaaacctggggcaagcgtgaaagtgagctgcaaggccagcggctacaccttcaccaactactacatgcactgggtgaggcaggcacctggtcaaggcctggagtggatgggcatgatcaaccccagcggaggagggaccagctacgcccagaagttccaaggccgcgtgaccatgactagggatacctccaccagcaccgtgtatatggagctgagcagcctgaggagcgaggacaccgccgtgtattactgcgccaggggcaatccctgggaactgagactggactactgggggcagggcaccctcgtgacagtgtcttctggaggcggaggatctggtggtggtggatctggcggcggaggaagtggtggcggaggctctgacatccagatgacccagagcccaagcagcctgagcgccagcgtgggcgacagggtgaccatcacctgccaggccagccaggacatctcaaactacctgaactggtatcaacagaagcccggcaaggcccccaagttgcttatctacgccgcgagcagcttgcaaagcggcgtgcccagcaggttcagcggtagtggcagtggcaccgactttaccctgaccataagcagtctgcagcccgaggacttcgccacctactactgccagcagtactacagctaccccttcaccttcggacccggaaccaaggtggacatcaaaggcggagggggatccgagcccaagagcagcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttataccctccctccttgccgtgaagagatgaccaagaatcaagtttctttatggtgcctcgtcaagggcttttaccctagcgacattgctgtggagtgggagtccaacggccagcccgagaacaactacaagaccaccccccccgtcctcgatagcgacggttctttctttttatattctaagctgaccgttgataagtcccggtggcagcaaggcaacgtgtttagctgctccgtgatgcacgaggctttacataatcactacacccagaagtccctctctttatcccccggtaag

SEQ ID NO: 685 - protein, 502 aa, Artificial Sequence
  Description of Artificial Sequence Synthetic polypeptide
MGWSCIILFLVATATGVHSQVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIKGGGGSEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 686 - DNA, 1591 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactccgaggtgcagcttcttgagtctggcggtggtctggtgaagcccggtggcagcctgcgcctcagctgtgccgcaagcggcttcagtttcagcagctactggatgagctgggtgaggcaggcacccggcaagggccttgagtggatcagctacatcagcggcgactctggctacaccaactacgccgacagcgttaagggcaggttcaccatcagcagggacgatagcaagaacaccctgtacctgcagatgaatagcctgaagaccgaagacaccgccgtgtactactgcgccagccacgattacggagactatggagagtacttccagcactggggccagggcaccctggtgacggtgagctctggaggcggaggatctggtggtggtggatctggcggcggaggaagtggtggcggaggctctgacatccagatgacccagagcccctccagtcttagcgccagcgtgggcgacagggtgaccatcacctgccaggccagccaggacatcagcaactacctgaattggtatcagcagaagcccggcaaggcccccaagctcctgatctacgccgcgagctcactgcagagcggcgtgcccagcaggttcagtggcagcggaagcgggaccgacttcacgctgaccattagcagcctccagcctgaggacttcgccacctactactgccagcaagccatcagcttccccctgactttcggccagagcaccaaggtggagatcaagggagggggatccgagcccaagagcagcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttataccctccctccttgccgtgaagagatgaccaagaatcaagtttctttatggtgcctcgtcaagggcttttaccctagcgacattgctgtggagtgggagtccaacggccagcccgagaacaactacaagaccaccccccccgtcctcgatagcgacggttctttctttttatattctaagctgaccgttgataagtcccggtggcagcaaggcaacgtgtttagctgctccgtgatgcacgaggctttacataatcactacacccagaagtccctctctttatcccccggtaag

SEQ ID NO: 687 - protein, 503 aa, Artificial Sequence
  Description of Artificial Sequence Synthetic polypeptide
MGWSCIILFLVATATGVHSEVQLLESGGGLVKPGGSLRLSCAASGFSFSSYWMSWVRQAPGKGLEWISYISGDSGYTNYADSVKGRFTISRDDSKNTLYLQMNSLKTEDTAVYYCASHDYGDYGEYFQHWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQAISFPLTFGQSTKVEIKGGGSEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 688 - DNA, 1486 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactcccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcgagcgccagcaccaagggaccctccgtgttccctctggccccctccagcaagtccacatccggaggcaccgccgctctgggatgtctggtgaaggactacttccccgagcccgtgacagtgtcttggaactctggcgctttaaccagcggcgtccatacattccccgctgttttacagagctccggcctctactctttatccagcgtggtgaccgtccctagctccagcctcggaacccagacatacatctgcaatgtcaaccacaagccctccaacaccaaggtggataagagagtggagcccaagagctgcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttgcaccctccctccttctcgtgaagagatgaccaagaatcaagtttctttatcctgcgcggtcaagggcttttaccctagcgacattgctgtggagtgggagtccaacggccagcccgagaacaactacaagaccaccccccccgtcctcgatagcgacggttctttctttttagtctctaagctgaccgttgataagtcccggtggcagcaaggcaacgtgtttagctgctccgtgatgcacgaggctttacataatcgtttcacccagaagtccctctctttatcccccggtaag

SEQ ID NO: 689 - protein, 468 aa, Artificial Sequence
  Description of Artificial Sequence Synthetic polypeptide
MGWSCIILFLVATATGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 690 - DNA, 2254 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactcccaggtgcagctggtgcagagcggagccgaggtgaagaaacctggggcaagcgtgaaagtgagctgcaaggccagcggctacaccttcaccaactactacatgcactgggtgaggcaggcacctggtcaaggcctggagtggatgggcatgatcaaccccagcggaggagggaccagctacgcccagaagttccaaggccgcgtgaccatgactagggatacctccaccagcaccgtgtatatggagctgagcagcctgaggagcgaggacaccgccgtgtattactgcgccaggggcaatccctgggaactgagactggactactgggggcagggcaccctcgtgacagtgtcttctggaggcggaggatctggtggtggtggatctggcggcggaggaagtggtggcggaggctctgacatccagatgacccagagcccaagcagcctgagcgccagcgtgggcgacagggtgaccatcacctgccaggccagccaggacatctcaaactacctgaactggtatcaacagaagcccggcaaggcccccaagttgcttatctacgccgcgagcagcttgcaaagcggcgtgcccagcaggttcagcggtagtggcagtggcaccgactttaccctgaccataagcagtctgcagcccgaggacttcgccacctactactgccagcagtactacagctaccccttcaccttcggacccggaaccaaggtggacatcaaaggcggagggggatccggcggagggggctctcaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcgagcgccagcaccaagggaccctccgtgttccctctggccccctccagcaagtccacatccggaggcaccgccgctctgggatgtctggtgaaggactacttccccgagcccgtgacagtgtcttggaactctggcgctttaaccagcggcgtccatacattccccgctgttttacagagctccggcctctactctttatccagcgtggtgaccgtccctagctccagcctcggaacccagacatacatctgcaatgtcaaccacaagccctccaacaccaaggtggataagagagtggagcccaagagctgcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttgcaccctccctccttctcgtgaagagatgaccaagaatcaagtttctttatcctgcgcggtcaagggcttttaccctagcgacattgctgtggagtgggagtccaacggccagcccgagaacaactacaagaccaccccccccgtcctcgatagcgacggttctttctttttagtctctaagctgaccgttgataagtcccggtggcagcaaggcaacgtgtttagctgctccgtgatgcacgaggctttacataatcgtttcacccagaagtccctctctttatcccccggtaag

SEQ ID NO: 691 - protein, 724 aa, Artificial Sequence
  Description of Artificial Sequence Synthetic polypeptide
MGWSCIILFLVATATGVHSQVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIKGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 692 - DNA, 2257 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactccgaggtgcagcttcttgagtctggcggtggtctggtgaagcccggtggcagcctgcgcctcagctgtgccgcaagcggcttcagtttcagcagctactggatgagctgggtgaggcaggcacccggcaagggccttgagtggatcagctacatcagcggcgactctggctacaccaactacgccgacagcgttaagggcaggttcaccatcagcagggacgatagcaagaacaccctgtacctgcagatgaatagcctgaagaccgaagacaccgccgtgtactactgcgccagccacgattacggagactatggagagtacttccagcactggggccagggcaccctggtgacggtgagctctggaggcggaggatctggtggtggtggatctggcggcggaggaagtggtggcggaggctctgacatccagatgacccagagcccctccagtcttagcgccagcgtgggcgacagggtgaccatcacctgccaggccagccaggacatcagcaactacctgaattggtatcagcagaagcccggcaaggcccccaagctcctgatctacgccgcgagctcactgcagagcggcgtgcccagcaggttcagtggcagcggaagcgggaccgacttcacgctgaccattagcagcctccagcctgaggacttcgccacctactactgccagcaagccatcagcttccccctgactttcggccagagcaccaaggtggagatcaagggagggggatccggcggagggggctctcaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcgagcgccagcaccaagggaccctccgtgttccctctggccccctccagcaagtccacatccggaggcaccgccgctctgggatgtctggtgaaggactacttccccgagcccgtgacagtgtcttggaactctggcgctttaaccagcggcgtccatacattccccgctgttttacagagctccggcctctactctttatccagcgtggtgaccgtccctagctccagcctcggaacccagacatacatctgcaatgtcaaccacaagccctccaacaccaaggtggataagagagtggagcccaagagctgcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttgcaccctccctccttctcgtgaagagatgaccaagaatcaagtttctttatcctgcgcggtcaagggcttttaccctagcgacattgctgtggagtgggagtccaacggccagcccgagaacaactacaagaccaccccccccgtcctcgatagcgacggttctttctttttagtctctaagctgaccgttgataagtcccggtggcagcaaggcaacgtgtttagctgctccgtgatgcacgaggctttacataatcgtttcacccagaagtccctctctttatcccccggtaag

SEQ ID NO: 693 - protein, 725 aa, Artificial Sequence
  Description of Artificial Sequence Synthetic polypeptide
MGWSCIILFLVATATGVHSEVQLLESGGGLVKPGGSLRLSCAASGFSFSSYWMSWVRQAPGKGLEWISYISGDSGYTNYADSVKGRFTISRDDSKNTLYLQMNSLKTEDTAVYYCASHDYGDYGEYFQHWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQAISFPLTFGQSTKVEIKGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 694 - DNA, 841 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactccggatccgagcccaagagcagcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttataccctccctccttgccgtgaagagatgaccaagaatcaagtttctttatggtgcctcgtcaagggcttttaccctagcgacattgctgtggagtgggagtccaacggccagcccgagaacaactacaagaccaccccccccgtcctcgatagcgacggttctttctttttatattctaagctgaccgttgataagtcccggtggcagcaaggcaacgtgtttagctgctccgtgatgcacgaggctttacataatcactacacccagaagtccctctctttatcccccggtaag

SEQ ID NO: 695 - protein, 253 aa, Artificial Sequence
  Description of Artificial Sequence Synthetic polypeptide
MGWSCIILFLVATATGVHSGSEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 696 - DNA, 2254 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactcccaggtgcagctggtgcagagcggagccgaggtgaagaaacctggggcaagcgtgaaagtgagctgcaaggccagcggctacaccttcaccaactactacatgcactgggtgaggcaggcacctggtcaaggcctggagtggatgggcatgatcaaccccagcggaggagggaccagctacgcccagaagttccaaggccgcgtgaccatgactagggatacctccaccagcaccgtgtatatggagctgagcagcctgaggagcgaggacaccgccgtgtattactgcgccaggggcaatccctgggaactgagactggactactgggggcagggcaccctcgtgacagtgtcttctggaggcggaggatctggtggtggtggatctggcggcggaggaagtggtggcggaggctctgacatccagatgacccagagcccaagcagcctgagcgccagcgtgggcgacagggtgaccatcacctgccaggccagccaggacatctcaaactacctgaactggtatcaacagaagcccggcaaggcccccaagttgcttatctacgccgcgagcagcttgcaaagcggcgtgcccagcaggttcagcggtagtggcagtggcaccgactttaccctgaccataagcagtctgcagcccgaggacttcgccacctactactgccagcagtactacagctaccccttcaccttcggacccggaaccaaggtggacatcaaaggcggagggggatccggcggagggggctctcaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcgagcgccagcaccaagggaccctccgtgttccctctggccccctccagcaagtccacatccggaggcaccgccgctctgggatgtctggtgaaggactacttccccgagcccgtgacagtgtcttggaactctggcgctttaaccagcggcgtccatacattccccgctgttttacagagctccggcctctactctttatccagcgtggtgaccgtccctagctccagcctcggaacccagacatacatctgcaatgtcaaccacaagccctccaacaccaaggtggataagagagtggagcccaagagctgcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttataccctccctccttctcgtgaagagatgaccaagaatcaagtttctttaacatgcctcgtcaagggcttttaccctagcgacattgctgtggagtgggagtccaacggccagcccgagaacaactacaagaccaccccccccgtcctcgatagcgacggttctttctttttatattctaagctgaccgttgataagtcccggtggcagcaaggcaacgtgtttagctgctccgtgatgcacgaggctttacataatcactacacccagaagtccctctctttatcccccggtaag

SEQ ID NO: 697 - protein, 724 aa, Artificial Sequence
  Description of Artificial Sequence Synthetic polypeptide
MGWSCIILFLVATATGVHSQVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIKGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 698 - DNA, 2254 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactcccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcgagcgccagcaccaagggaccctccgtgttccctctggccccctccagcaagtccacatccggaggcaccgccgctctgggatgtctggtgaaggactacttccccgagcccgtgacagtgtcttggaactctggcgctttaaccagcggcgtccatacattccccgctgttttacagagctccggcctctactctttatccagcgtggtgaccgtccctagctccagcctcggaacccagacatacatctgcaatgtcaaccacaagccctccaacaccaaggtggataagagagtggagcccaagagctgcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttataccctccctccttctcgtgaagagatgaccaagaatcaagtttctttaacatgcctcgtcaagggcttttaccctagcgacattgctgtggagtgggagtccaacggccagcccgagaacaactacaagaccaccccccccgtcctcgatagcgacggttctttctttttatattctaagctgaccgttgataagtcccggtggcagcaaggcaacgtgtttagctgctccgtgatgcacgaggctttacataatcactacacccagaagtccctctctttatcccccggtaagggcggagggggatccggcggagggggctctcaggtgcagctggtgcagagcggagccgaggtgaagaaacctggggcaagcgtgaaagtgagctgcaaggccagcggctacaccttcaccaactactacatgcactgggtgaggcaggcacctggtcaaggcctggagtggatgggcatgatcaaccccagcggaggagggaccagctacgcccagaagttccaaggccgcgtgaccatgactagggatacctccaccagcaccgtgtatatggagctgagcagcctgaggagcgaggacaccgccgtgtattactgcgccaggggcaatccctgggaactgagactggactactgggggcagggcaccctcgtgacagtgtcttctggaggcggaggatctggtggtggtggatctggcggcggaggaagtggtggcggaggctctgacatccagatgacccagagcccaagcagcctgagcgccagcgtgggcgacagggtgaccatcacctgccaggccagccaggacatctcaaactacctgaactggtatcaacagaagcccggcaaggcccccaagttgcttatctacgccgcgagcagcttgcaaagcggcgtgcccagcaggttcagcggtagtggcagtggcaccgactttaccctgaccataagcagtctgcagcccgaggacttcgccacctactactgccagcagtactacagctaccccttcaccttcggacccggaaccaaggtggacatcaaa

SEQ ID NO: 699 - protein, 724 aa, Artificial Sequence
  Description of Artificial Sequence Synthetic polypeptide
MGWSCIILFLVATATGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIK

SEQ ID NO: 700 - DNA, 2260 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactccgaggtgcagcttcttgagtctggcggtggtctggtgaagcccggtggcagcctgcgcctcagctgtgccgcaagcggcttcagtttcagcagctactggatgagctgggtgaggcaggcacccggcaagggccttgagtggatcagctacatcagcggcgactctggctacaccaactacgccgacagcgttaagggcaggttcaccatcagcagggacgatagcaagaacaccctgtacctgcagatgaatagcctgaagaccgaagacaccgccgtgtactactgcgccagccacgattacggagactatggagagtacttccagcactggggccagggcaccctggtgacggtgagctctggaggcggaggatctggtggtggtggatctggcggcggaggaagtggtggcggaggctctgacatccagatgacccagagcccctccagtcttagcgccagcgtgggcgacagggtgaccatcacctgccaggccagccaggacatcagcaactacctgaattggtatcagcagaagcccggcaaggcccccaagctcctgatctacgccgcgagctcactgcagagcggcgtgcccagcaggttcagtggcagcggaagcgggaccgacttcacgctgaccattagcagcctccagcctgaggacttcgccacctactactgccagcaagccatcagcttccccctgactttcggccagagcaccaaggtggagatcaagggcggagggggatccggcggagggggctctcaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcgagcgccagcaccaagggaccctccgtgttccctctggccccctccagcaagtccacatccggaggcaccgccgctctgggatgtctggtgaaggactacttccccgagcccgtgacagtgtcttggaactctggcgctttaaccagcggcgtccatacattccccgctgttttacagagctccggcctctactctttatccagcgtggtgaccgtccctagctccagcctcggaacccagacatacatctgcaatgtcaaccacaagccctccaacaccaaggtggataagagagtggagcccaagagctgcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttataccctccctccttctcgtgaagagatgaccaagaatcaagtttctttaacatgcctcgtcaagggcttttaccctagcgacattgctgtggagtgggagtccaacggccagcccgagaacaactacaagaccaccccccccgtcctcgatagcgacggttctttctttttatattctaagctgaccgttgataagtcccggtggcagcaaggcaacgtgtttagctgctccgtgatgcacgaggctttacataatcactacacccagaagtccctctctttatcccccggtaag

SEQ ID NO: 701 - protein, 726 aa, Artificial Sequence
  Description of Artificial Sequence Synthetic polypeptide
MGWSCIILFLVATATGVHSEVQLLESGGGLVKPGGSLRLSCAASGFSFSSYWMSWVRQAPGKGLEWISYISGDSGYTNYADSVKGRFTISRDDSKNTLYLQMNSLKTEDTAVYYCASHDYGDYGEYFQHWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQAISFPLTFGQSTKVEIKGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 702 - DNA, 2260 bp, Artificial Sequence
  Description of Artificial Sequence Synthetic polynucleotide
atgggatggagctgtatcatcctcttcttggtagcaacagctacaggtaaggggctcacagtagcaggcttgaggtctggacatatatatgggtgacaatgacatccactttgcctttctctccacaggtgtacactcccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcgagcgccagcaccaagggaccctccgtgttccctctggccccctccagcaagtccacatccggaggcaccgccgctctgggatgtctggtgaaggactacttccccgagcccgtgacagtgtcttggaactctggcgctttaaccagcggcgtccatacattccccgctgttttacagagctccggcctctactctttatccagcgtggtgaccgtccctagctccagcctcggaacccagacatacatctgcaatgtcaaccacaagccctccaacaccaaggtggataagagagtggagcccaagagctgcgacaaaacccatacatgccccccttgtcccgctcccgaactgctgggcggacctagcgtctttttattcccccccaagcccaaggacactttaatgatctctcgtacccccgaagtgacttgtgtcgtcgtggacgtgagccacgaggaccccgaggtcaagttcaactggtacgttgatggcgtggaagtgcacaacgccaagaccaaacctcgtgaagagcagtaccagtccacatatcgggtggtgtccgtgctgaccgtgctgcaccaagattggctgaacggcaaggagtacaagtgtaaagtgtccaacaaggccctccccgcccccatcgagaagaccatcagcaaagccaagggccagcctagggagcctcaagtttataccctccctccttctcgtgaagagatgaccaagaatcaagtttctttaacatgcctcgtcaagggcttttaccctagcgacattgctgtggagtgggagtccaacggccagcccgagaacaactacaagaccaccccccccgtcctcgatagcgacggttctttctttttatattctaagctgaccgttgataagtcccggtggcagcaaggcaacgtgtttagctgctccgtgatgcacgaggctttacataatcactacacccagaagtccctctctttatcccccggtaagggcggagggggatccggcggagggggctctgaggtgcagcttcttgagtctggcggtggtctggtgaagcccggtggcagcctgcgcctcagctgtgccgcaagcggcttcagtttcagcagctactggatgagctgggtgaggcaggcacccggcaagggccttgagtggatcagctacatcagcggcgactctggctacaccaactacgccgacagcgttaagggcaggttcaccatcagcagggacgatagcaagaacaccctgtacctgcagatgaatagcctgaagaccgaagacaccgccgtgtactactgcgccagccacgattacggagactatggagagtacttccagcactggggccagggcaccctggtgacggtgagctctggaggcggaggatctggtggtggtggatctggcggcggaggaagtggtggcggaggctctgacatccagatgacccagagcccctccagtcttagcgccagcgtgggcgacagggtgaccatcacctgccaggccagccaggacatcagcaactacctgaattggtatcagcagaagcccggcaaggcccccaagctcctgatctacgccgcgagctcactgcagagcggcgtgcccagcaggttcagtggcagcggaagcgggaccgacttcacgctgaccattagcagcctccagcctgaggacttcgccacctactactgccagcaagccatcagcttccccctgactttcggccagagcaccaaggtggagatcaag

SEQ ID NO: 703 - protein, 726 aa, Artificial Sequence
  Description of Artificial Sequence Synthetic polypeptide
MGWSCIILFLVATATGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVKPGGSLRLSCAASGFSFSSYWMSWVRQAPGKGLEWISYISGDSGYTNYADSVKGRFTISRDDSKNTLYLQMNSLKTEDTAVYYCASHDYGDYGEYFQHWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQAISFPLTFGQSTKVEIK

SEQ ID NO: 704 - DNA, 705 bp, Artificial Sequence
  Description of Artificial Sequence Synthetic polynucleotide
atggacatgagggtgcccgctcagctgctgggactgctgctgctgtggctgcccggtgcgcgctgccaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaaccgtacggtggctgccccctccgtgttcatcttccccccctccgacgagcagctgaagtccggcaccgcctccgtggtgtgtttactgaacaacttctaccctcgtgaggccaaggtgcagtggaaggtggacaacgctttacagtccggcaacagccaagaatccgtgaccgagcaagattccaaggactccacatactctttatccagcactttaactttatccaaggccgactacgagaagcacaaggtgtacgcttgtgaggtgacccaccaaggtctgagctcccccgtgaccaagtccttcaatcgtggcgagtgc

SEQ ID NO: 705 - protein, 235 aa, Artificial Sequence
  Description of Artificial Sequence Synthetic polypeptide
MDMRVPAQLLGLLLLWLPGARCQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 706 - protein, 246 aa, Artificial Sequence
  Description of Artificial Sequence Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIKGGGGSGGGGSGGGGSGGGGSQVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSS

SEQ ID NO: 707 - protein, 243 aa, Artificial Sequence
  Description of Artificial Sequence Synthetic polypeptide
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSVEGGSGGSGGSGGSGGVDDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 708 - protein, 254 aa, Artificial Sequence
  Description of Artificial Sequence Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 709 - protein, 253 aa, Artificial Sequence
  Description of Artificial Sequence Synthetic polypeptide
DIQMTQSPSSLSASVGDRVTITCRASQSISSWLAWYQQKPGKAPKLLIYAASTLQSGVPSRFSGSGSGTDFTLTISSLQPEDFATYYCQQSYSIPLTFGGGTKVEIKGGGGSGGGGSGGGGSGGGGSQVQLVQSGAEVKKPGSSVKVSCKASGGTFSNFGVSWLRQAPGQGLEWMGGIIPILGTANYAQKFQGRVTITADESTSTAYMELSSLRSEDTAVYYCATPTNSGYYGPYYYYGMDVWGQGTTVTVSS

SEQ ID NO: 710 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 711 - protein, 246 aa, Artificial Sequence
  Description of Artificial Sequence Synthetic polypeptide
QVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIK

SEQ ID NO: 712 - protein, 213 aa, Artificial Sequence
  Description of Artificial Sequence Synthetic polypeptide
DIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 713 - protein, 216 aa, Artificial Sequence
  Description of Artificial Sequence Synthetic polypeptide
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 714 - protein, 253 aa, Artificial Sequence
  Description of Artificial Sequence Synthetic polypeptide
QVQLVQSGAEVKKPGSSVKVSCKASGGTFSNFGVSWLRQAPGQGLEWMGGIIPILGTANYAQKFQGRVTITADESTSTAYMELSSLRSEDTAVYYCATPTNSGYYGPYYYYGMDVWGQGTTVTVSSGGGGSGGGGSGGGGSGGGGSDIQMTQSPSSLSASVGDRVTITCRASQSISSWLAWYQQKPGKAPKLLIYAASTLQSGVPSRFSGSGSGTDFTLTISSLQPEDFATYYCQQSYSIPLTFGGGTKVEIK

SEQ ID NO: 715 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 716 - protein, 237 aa, Artificial Sequence
  Description of Artificial Sequence Synthetic polypeptide
GGGGSEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 717 - protein, 234 aa, Artificial Sequence
  Description of Artificial Sequence Synthetic polypeptide
GSEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 718 - protein, 246 aa, Artificial Sequence
  Description of Artificial Sequence Synthetic polypeptide
QVQLVQSGAEVKKPGASVKVSCKASGYTFTNYYMHWVRQAPGQCLEWMGMINPSGGGTSYAQKFQGRVTMTRDTSTSTVYMELSSLRSEDTAVYYCARGNPWELRLDYWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGCGTKVDIK

SEQ ID NO: 719 - protein, 253 aa, Artificial Sequence
  Description of Artificial Sequence Synthetic polypeptide
QVQLVQSGAEVKKPGSSVKVSCKASGGTFSNFGVSWLRQAPGQCLEWMGGIIPILGTANYAQKFQGRVTITADESTSTAYMELSSLRSEDTAVYYCATPTNSGYYGPYYYYGMDVWGQGTTVTVSSGGGGSGGGGSGGGGSGGGGSDIQMTQSPSSLSASVGDRVTITCRASQSISSWLAWYQQKPGKAPKLLIYAASTLQSGVPSRFSGSGSGTDFTLTISSLQPEDFATYYCQQSYSIPLTFGGGTKVEIK

SEQ ID NO: 720 - protein, 242 aa, Artificial Sequence
  Description of Artificial Sequence Synthetic polypeptide
GGCGGGGSGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 721 - protein, 236 aa, Artificial Sequence
  Description of Artificial Sequence Synthetic polypeptide
QVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIK

SEQ ID NO: 722 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 723 - protein, 237 aa, Artificial Sequence
  Description of Artificial Sequence Synthetic polypeptide
GGCGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 724 - protein, 242 aa, Artificial Sequence
  Description of Artificial Sequence Synthetic polypeptide
GGGCGGGSGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 725 - protein, 15 aa, Artificial Sequence
  Description of Artificial Sequence Synthetic peptide
GGGCGGGSGSGGGGS

SEQ ID NO: 726 - protein, 238 aa, Artificial Sequence
  Description of Artificial Sequence Synthetic polypeptide
GGGCGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 727 - protein, 243 aa, Artificial Sequence
  Description of Artificial Sequence Synthetic polypeptide
GGGCGGGGSGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 728 - protein, 449 aa, Artificial Sequence
  Description of Artificial Sequence Synthetic polypeptide
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 729 - protein, 13 aa, Homo sapiens
KSCDKTHTCPPCP

SEQ ID NO: 730 - protein, 5 aa, Artificial Sequence
  Description of Artificial Sequence Synthetic peptide
GGGGS

SEQ ID NO: 731 - protein, 8 aa, Artificial Sequence
  Description of Artificial Sequence Synthetic peptide
GSGGGGCP

SEQ ID NO: 732 - protein, 4 aa, Homo sapiens
CPPC

SEQ ID NO: 733 - protein, 122 aa, Artificial Sequence [sequence omitted]

SEQ ID NO: 734 - protein, 107 aa, Artificial Sequence [sequence omitted]

SEQ ID NO: 735 - protein, 237 aa, Artificial Sequence
  Description of Artificial Sequence Synthetic polypeptide
GGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 736 - protein, 462 aa, Artificial Sequence
  Description of Artificial Sequence Synthetic polypeptide
GGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 737 - protein, 214 aa, Artificial Sequence
  Description of Artificial Sequence Synthetic polypeptide
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 738 - protein, 241 aa, Artificial Sequence
  Description of Artificial Sequence Synthetic polypeptide
EVQLLESGGGLVQPGGSLRLSCAASGFTFSDYYMSWVRQAPGKGLEWVSGINWNGGSTGYADSVKGRFTISRDNSKNTLYLQMNSLRAEDTAVYYCARVEQGYDIYYYYYMDVWGKGTTVTVSSGGGGSGGGGSDIQMTQSPSSLSASVGDRVTITCRASQSISSYLNWYQQKPGKAPKLLIYKASSLESGVPSRFSGSGSGTDFTLTISSLQPEDFATYYCQQSYSAPYTFGPGTKVDIK

SEQ ID NO: 739 - protein, 459 aa, Artificial Sequence
  Description of Artificial Sequence Synthetic polypeptide
GGGGSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 740 - protein, 213 aa, Artificial Sequence; identical to SEQ ID NO: 712
  Description of Artificial Sequence Synthetic polypeptide